SEQUENCE LISTING (5 sequences)

SEQ ID NO: 1 - protein, 20 aa, Homo sapiens
ESTEDYNVACILTLPPYQRR

SEQ ID NO: 2 - protein, 20 aa, Homo sapiens
ESPDGNNVACILTLPPYQRR

SEQ ID NO: 3 - protein, 20 aa, Homo sapiens
NSFLNYNVSCILTMPQYMRQ

SEQ ID NO: 4 - protein, 20 aa, Homo sapiens
HCQQKYNVSCIMILPQYQRK

SEQ ID NO: 5 - protein, 20 aa, Homo sapiens
LCQQKYNVSCIMIMPQHQRQ

(19) United States
(12) Patent Application Publication
(10) Pub. No.: US 2025/0122182 A1
(43) Pub. Date: Apr. 17, 2025

(54) MYST Inhibitors

(71) Applicant: Isosterix, Inc., San Carlos, CA (US)

(72) Inventors: Roopa Rai, San Carlos, CA (US); Anjali Pandey, Freemont, CA (US); Leslie J. Holsinger, Encinitas, CA (US)

(21) Appl. No.: 18/898,082

(22) Filed: Sep. 26, 2024

Related U.S. Application Data
(60) Provisional application No. 63/585,817, filed on Sep. 27, 2023.
(60) Provisional application No. 63/558,419, filed on Feb. 27, 2024.
(60) Provisional application No. 63/655,961, filed on Jun. 4, 2024.

(51) Int. Cl. C07D 413/14; A61K 31/4162; A61K 31/4178; A61K 31/423; A61K 31/4245; A61K 31/427; A61K 31/4439; A61K 31/4709; A61K 31/5377; A61K 31/55; A61P 35/00; C07D 413/06; C07D 413/12; C07D 417/12; C07D 487/04; C07D 498/04; C07D 515/04

(52) U.S. Cl. CPC C07D 413/14; A61K 31/4162; A61K 31/4178; A61K 31/423; A61K 31/4245; A61K 31/427; A61K 31/4439; A61K 31/4709; A61K 31/5377; A61K 31/55; A61P 35/00; C07D 413/06; C07D 413/12; C07D 417/12; C07D 487/04; C07D 498/04; C07D 515/04

(57) ABSTRACT

Provided herein are compounds that are MYST inhibitors, and pharmaceutically acceptable derivatives thereof. Also provided are pharmaceutical compositions containing the compounds and methods of using the compounds for treating a subject with a hyperproliferative disease including cancer.

### RELATED APPLICATIONS

This application claims the benefit of priority to U.S. provisional patent application nos. 63/585,817, filed Sep. 27, 2023, 63/558,419, filed Feb. 27, 2024, and 63/655,961, filed Jun. 4, 2024, the disclosures of which are incorporated herein by reference in their entireties.

### SEQUENCE LISTING

This application contains a sequence listing, which is being submitted herewith as an XML filed named “ISTX104WO_SL.xml”, created on Sep. 11, 2024, size 5406 bytes, which is incorporated by reference herein in its entirety.

### FIELD

Provided herein are compounds and compositions for inhibiting the MYST family of lysine acetyltransferases (KATs). In one embodiment, provided are methods of treating diseases, including hyperproliferative diseases, with the compounds and compositions provided herein. In another embodiment, the hyperproliferative disease is cancer.

### BACKGROUND

The MYST family of enzymes are KATs and include KAT6A (also known as MOZ or MYST3), KAT6B (also known as MORF or MYST4), KAT5 (also known as Tip60), KAT7 (also known as, HBO1 or MYST2) and KAT8 (also known as, MOF or MYST1) (see, e.g., Wiesel-Motiuk, et al., Drug Resistance Updates 2020, 53, 100729; Wapenaar, et al., Clinical Epigenetics 2016, 8(59), 1 (DOI 10.1186/s13148-016-0225-2)). The MYST family of KATs is particularly important in regulation of the cell cycle (Carrozza, et al., Trends in Genetics 2003, 19, 321-329).

Dysregulation of the expression of KAT6 proteins supports tumor progression (see, e.g., Trisciuoglio et al., Stem Cells Int. 2018, Article ID 8908751 (https://doi.org/10.1155/2018/8908751)). Dysregulation through gene amplification, overexpression or mutation of KAT6A has been documented in multiple cancer types including breast, lung adenocarcinoma, ovarian, colon and rectal adenocarcinoma, and uterine cervix (see, e.g., Huang, et al., Molecular and Cellular Biology 2016, 63, 1900-1907; Zack, et al., Nature Genetics 2013, 45, 1134-1140). It is reported that the locus that contains the gene for KAT6A (8p11-p12 amplicon) is the 12^{th} most commonly amplified region of the genome across all cancer types (see, e.g., Zack, et al., 2013, supra). In acute myeloid leukemia (AML), recurrent oncogenic fusions of KAT6A have been documented that can drive transformation to a malignant state (see, e.g., Sheikh, et al., Blood 2015, 125, 1910-1921). Translocation of KAT6A and fusion to a partner such as CBP, p300, TIF2 and NCOA3 are known to lead to aggressive forms of AML (see, e.g., Sheikh, et al., 2015, supra).

In addition to the role that KATs play in epigenetic regulation, KATs also acetylate non-histone proteins. Through modification of the acetylation state of non-histone proteins, KATs are involved modulating protein function and stability, protein-protein and protein-DNA interactions, and regulation of enzyme activity (see, e.g., Glozak, et al., Gene 2005, 363, 15-23; Das, et al., IUBMB Life 2005, 57, 137-148).

Thus, there is a need for MYST inhibitors for treatment of hyperproliferative diseases, including cancer.

### SUMMARY

Provided herein is a compound of Formula I:

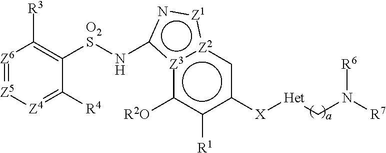

or a pharmaceutically acceptable derivative thereof, where R¹-R⁴, R⁶, R⁷, a, Het, X and Z¹-Z⁶ are as defined elsewhere herein.

Also provided herein is a compound of Formula II:

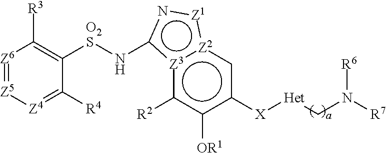

or a pharmaceutically acceptable derivative thereof, where R¹-R⁴, R⁶, R⁷, a, Het, X and Z¹-Z⁶ are as defined elsewhere herein.

Also provided herein is a compound of Formula III:

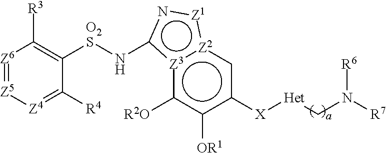

or a pharmaceutically acceptable derivative thereof, where R¹-R⁴, R⁶, R⁷, a, Het, X and Z¹-Z⁶ are as defined elsewhere herein.

Pharmaceutical compositions containing a compound of Formula I, II or III and a pharmaceutically acceptable excipient are also provided.

In one embodiment, the compounds of Formula I, II or III provided herein inhibit one or more of the MYST family of lysine acetyl transferases. In another embodiment, the compounds provided herein inhibit KAT6A. In another embodiment, the compounds provided herein are covalent inhibitors of KAT6A. Without being bound by any theory, in one embodiment, the compounds provided herein inhibit KAT6A through covalent modification of a cysteine residue at or near the Ac-CoA binding site. In another embodiment, the compounds provided herein are non-covalent inhibitors of KAT6A.

In other embodiments, provided are methods of treatment of a hyperproliferative disease by administering a compound or composition provided herein. In one embodiment, the hyperproliferative disease is cancer. In another embodiment, the cancer is breast cancer, lung cancer, ovarian cancer, colon cancer, prostate cancer, uterine/cervical cancer or leukemia. In another embodiment of the methods provided herein, the compounds of Formula I are used as single agents or in combination with a standard of care treatment for a particular cancer.

In another embodiment, provided are methods of inhibiting a MYST enzyme by contacting the MYST enzyme or a composition containing a MYST enzyme with a compound or composition provided herein. In another embodiment, provided are methods of inhibiting KAT6A by contacting KAT6A or a composition containing KAT6A with a compound or composition provided herein.

### DETAILED DESCRIPTION

### I. Definitions

To facilitate understanding of the disclosure set forth herein, a number of terms are defined below.

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behavior of such compounds, compositions and mixtures. Biological activities can be observed in in vitro systems designed to test for such activities.

As used herein, pharmaceutically acceptable derivatives of a compound include, but are not limited to, salts, esters, enol ethers, enol esters, acetals, ketals, orthoesters, hemiacetals, hemiketals, acids, bases, clathrates, solvates or hydrates thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating a hyperproliferative disease.

As used herein, amelioration of the symptoms of a particular disorder by administration of a particular compound or pharmaceutical composition refers to any lessening, whether permanent or temporary, lasting or transient that can be attributed to or associated with administration of the compound or pharmaceutical composition.

As used herein, and unless otherwise indicated, the terms “manage,” “managing” and “management” encompass preventing the recurrence of the specified disease or disorder in a subject who has already suffered from the disease or disorder, and/or lengthening the time that a subject who has suffered from the disease or disorder remains in remission. The terms encompass modulating the threshold, development and/or duration of the disease or disorder, or changing the way that a subject responds to the disease or disorder.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e., C₁-C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e., C₁-C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e., C₁-C₁₀ means one to ten carbons). Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having six or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain hydrocarbon radical, consisting of a heteroatom selected from the group consisting of O, N, P, Si and S, and wherein the nitrogen and sulfur atoms may optionally be oxidized and the nitrogen atom may have an alkyl substituent to fulfill valency and/or may optionally be quaternized. The heteroatom(s) O, N, P, Si and S may be placed at any interior position of the heteroalkyl group. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CHO—CH₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For alkylene and heteroalkylene linking groups, no orientation of the linking group is implied by the direction in which the formula of the linking group is written. For example, the formula —C(O)₂R′— represents both —C(O)₂R′—and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic, spirocyclic and bridged bicyclic groups. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, norbornanyl, bicyclo[2.2.2]octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo[2.2.2]octanyl, and the like.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like. Similarly, the terms “cycloalkenyl”, “cycloalkynyl”, “heterocycloalkenyl” and “heterocycloalkynyl”, by themselves or in combination with other terms, represent, unless otherwise stated, cyclic versions of “alkenyl”, “alkynyl”, “heteroalkenyl” and “heteroalkynyl”, respectively, including bicyclic, tricyclic, spirocyclic and bridged bicyclic groups.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for aryl and heteroaryl ring systems may be selected from the group of acceptable substituent moieties described herein. The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, =NR′, =N—OR′, —NR′R″, —SR′, halo, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′″)=NR″″, —NR—C(NR′R″)=NR″′, —S(O)R′, —S(O)₂R′, —S(O)₂NR′R″, —NRSO₂R′, —NRSO₂NR′R″, —CN and —NO₂ in a number ranging from zero to the number of hydrogen atoms in such radical. In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R″′ and R″″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′ and R″″ groups when more than one of these groups is present. When R′ and R″ are attached to the same nitrogen atom, they can be combined with the nitrogen atom to form a 4-, 5-, 6-, or 7-membered ring. For example, —NR′R¹¹ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R″′ and R″″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R′ and R″″ groups when more than one of these groups is present.

Two of the substituent moieties on adjacent atoms of an aryl or heteroaryl ring may optionally form a ring of the formula -Q′-C(O)—(CRR′)_{q}-Q″-, wherein Q′ and Q″ are independently —NR′, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR′, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(CR″R″′)_{d}—, where s and d are independently integers of from 0 to 3, and X′ is —O—, —NR′—, —S—, —S(O)—, —S(O)₂—, or —S(O)₂NR′—. The substituent moieties R, R′, R″ and R″′ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds provided herein, whether radioactive or not, are encompassed within the scope of the present disclosure.

### II. Compounds for Use in Compositions and Methods

In one embodiment, provided is a compound of Formula I for use in the compositions and methods provided herein:

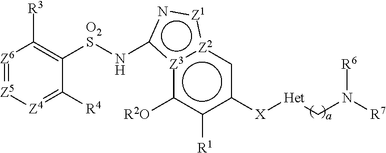

or a pharmaceutically acceptable derivative thereof, wherein:
a is 0-2;
R¹ and R² together form alkylene;
R³ is —O—(CH₂)_{b}-R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR²OR²¹.

In one embodiment, provided is a compound of Formula Ia for use in the compositions and methods provided herein:

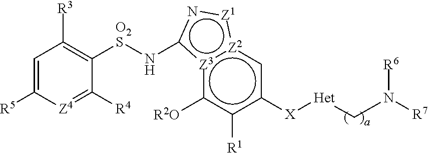

or a pharmaceutically acceptable derivative thereof, wherein:
a is 0-2;
R¹ and R² together form alkylene;
R³ is —O—(CH₂)_{b}-R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;

R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is O, S(O)ₓ or CH₂, where x is 0-2;

Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR²OR²¹.

In another embodiment, the compounds for use in the compositions and methods provided herein have Formula I or Ia, wherein:
R⁸ is H, CN, OH, alkoxy, aryl, heteroaryl, NR⁹R¹⁰ or C(O)R¹¹;
R⁹ is H and R¹⁰ is H or C(O)R¹³; where R¹³ is alkyl or alkoxy; and
R¹¹ is alkyl or alkoxy;
R⁵ and R¹⁶ are selected from (i) or (ii):
(i) R⁵ and R¹⁶ are each independently H, cyano, halo, alkoxy, alkyl, alkenyl or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I or Ia, wherein:
R⁸ is H, CN, OH, alkoxy, aryl, NR⁹R¹⁰ or C(O)R¹¹;
R⁹ is H and R¹⁰ is C(O)R¹³; where R¹³ is alkyl; and
R¹¹ is alkoxy;
R⁵ and R¹⁶ are selected from (i) or (ii):
(i) R⁵ is H, cyano, alkoxy or alkyl, and R¹⁶ is H, alkyl, alkoxy, halo or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I or Ia, wherein R¹ and R² together form C₂₋₄alkylene.

In another embodiment, the compounds for use in the compositions and methods provided herein have Formula I or Ia, wherein R¹ and R² together form propylene.

In another embodiment, the compounds for use in the compositions and methods provided herein have Formula I:

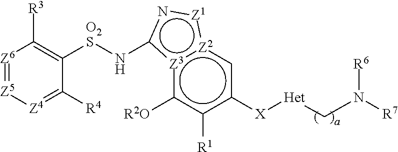

or a pharmaceutically acceptable derivative thereof, wherein:
a is 0-2;
R¹ and R² are selected from (i) or (ii):
(i) R¹ is H, alkyl, haloalkyl or halo; and R² is alkyl, cycloalkyl or haloalkyl, or
(ii) R¹ and R² together form alkylene;
R³ is —O—(CH₂)_{b}-R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is CR¹⁶;
Z⁵ is CR⁵;
Z⁶ is N or CH;
R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula Ia:

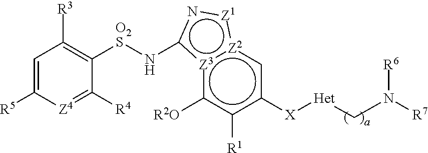

or a pharmaceutically acceptable derivative thereof, wherein:
a is 0-2;
R¹ and R² are selected from (i) or (ii):
(i) R¹ is H, alkyl, haloalkyl or halo; and R² is alkyl, cycloalkyl or haloalkyl, or
(ii) R¹ and R² together form alkylene;
R³ is —O—(CH₂)_{b}-R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹¹ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is CR¹⁶;
R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula I or Ia, wherein:
R¹ and R² are selected from (i) or (ii):
(i) R¹ is H; and R² is alkyl, cycloalkyl or haloalkyl, or
(ii) R¹ and R² together form alkylene;
R⁸ is H, CN, OH, aryl, heteroaryl, NR⁹R¹⁰ or C(O)R¹;
R⁹ and R¹⁰ are each independently H or C(O)R¹³; where R¹³ is alkyl or alkoxy;
R¹¹ is alkyl or alkoxy;
R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I or Ia, wherein:
R¹ and R² are selected from (i) or (ii):
(i) R¹ is H; and R² is alkyl or haloalkyl, or
(ii) R¹ and R² together form alkylene;
R⁸ is H, CN, OH, aryl, NR⁹R¹⁰ or C(O)R¹;
R⁹ and R¹⁰ are each independently H or C(O)R¹³; where R¹³ is alkyl;
R¹¹ is alkoxy;
R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I or Ia, wherein R⁵ and R¹⁶ together form C₃₋₅alkylene.

In another embodiment, the compounds for use in the compositions and methods provided herein have Formula I or Ia, wherein R⁵ and R¹⁶ together form propylene.

In another embodiment, the compounds for use in the compositions and methods provided herein have Formula I or Ia, wherein X is O or S(O)ₓ, where x is 0-2. In another embodiment, the compounds for use in the compositions and methods provided herein have Formula I or Ia, wherein X is O or S.

In another embodiment, the compounds for use in the compositions and methods provided herein have Formula I:

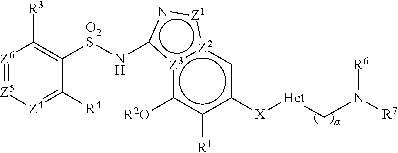

or a pharmaceutically acceptable derivative thereof, wherein:
a is 0-2;
R¹ and R² are selected from (i) or (ii):
(i) R¹ is H, alkyl, haloalkyl or halo; and R² is alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O or S(O)ₓ, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula Ia:

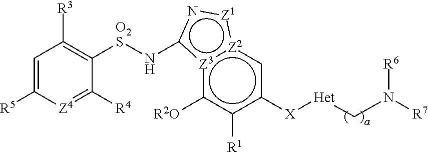

or a pharmaceutically acceptable derivative thereof, wherein:
a is 0-2;
R¹ and R² are selected from (i) or (ii):
(i) R¹ is H, alkyl, haloalkyl or halo; and R² is alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is O or S(O)ₓ, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula I or Ia, wherein:

R¹ and R² are selected from (i) or (ii):
(i) R¹ is H; and R² is alkyl, cycloalkyl or haloalkyl, or
(ii) R¹ and R² together form alkylene;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
R⁵ and R¹⁶ are selected from (i) or (ii):
(i) R⁵ and R¹⁶ are each independently H, cyano, halo, alkoxy, alkyl, alkenyl or alkynyl, or
(ii) R⁵ and R¹⁶ together form alkylene;
X is O;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I or Ia, wherein:
R¹ and R² are selected from (i) or (ii):
(i) R¹ is H; and R² is alkyl or haloalkyl, or
(ii) R¹ and R² together form alkylene;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
R⁵ and R¹⁶ are selected from (i) or (ii):
(i) R⁵ is H, cyano, alkoxy or alkyl, and R¹⁶ is H, alkyl, alkoxy, halo or alkynyl, or
(ii) R⁵ and R¹⁶ together form alkylene;
X is O;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula Ia:

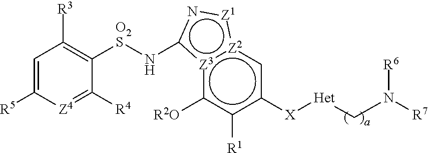

or a pharmaceutically acceptable derivative thereof, wherein:
a is 0-2;
R¹ and R² are selected from (i) or (ii):
(i) R¹ is H, alkyl, haloalkyl or halo; and R² is alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶, where R¹⁶ is H, alkyl, haloalkyl, alkoxy, halo, alkenyl or alkynyl;
R⁵ is H, alkyl, haloalkyl, alkoxy, halo, alkenyl or alkynyl;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula I or Ia, wherein:
R¹ and R² are selected from (i) or (ii):
(i) R¹ is H; and R² is alkyl, cycloalkyl or haloalkyl, or
(ii) R¹ and R² together form alkylene;
R⁵ is alkenyl or alkynyl;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I or Ia, wherein:
R¹ and R² are selected from (i) or (ii):
(i) R¹ is H; and R² is alkyl or haloalkyl, or
(ii) R¹ and R² together form alkylene;
R⁵ is alkynyl;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I:

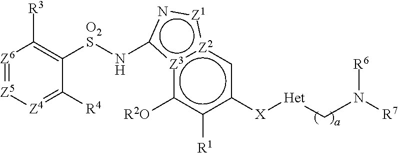

or a pharmaceutically acceptable derivative thereof, wherein:
a is 0-2;
R¹ is H, alkyl, haloalkyl or halo;
R² and R⁴ together form heteroalkylene;
R³ is —O—(CH₂)_{b}—R¹;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula Ia:

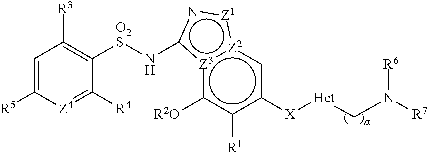

or a pharmaceutically acceptable derivative thereof, wherein:
a is 0-2;
R¹ is H, alkyl, haloalkyl or halo;
R² and R⁴ together form heteroalkylene;
R³ is —O—(CH₂)_{b}—R¹;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula I or Ia, wherein:
R¹ is H;
R² and R⁴ together form —O—(CH₂)ᵣ or —O—(CH₂)₁₋₃—O—(CH₂)₁₋₃—, where r is an integer from 2 to 5 and the terminal 0 is attached to the R⁴ position of Formula I;
R⁸ is H, CN, OH, aryl, heteroaryl, NR⁹R¹⁰ or C(O)R¹;
R⁹ and R¹⁰ are each independently H or C(O)R¹³; where R¹³ is alkyl or alkoxy;
R¹¹ is alkyl or alkoxy;
R⁵ and R¹⁶ are selected from (i) or (ii):
(i) R⁵ and R¹⁶ are each independently H, cyano, halo, alkoxy, alkyl, alkenyl or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I or Ia, wherein:
R¹ is H;
R² and R⁴ together form —O—(CH₂)ᵣ or —O—(CH₂)₁₋₃—O—(CH₂)₁₋₃—, where r is an integer from 2 to 5 and the terminal O is attached to the R⁴ position of Formula I;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
R⁵ and R¹⁶ are selected from (i) or (ii):
(i) R⁵ is H, cyano, alkoxy or alkyl, and R¹⁶ is H, alkyl, alkoxy, halo or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I:

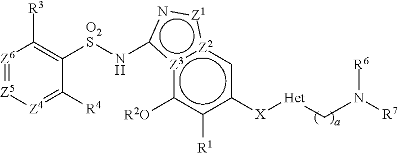

or a pharmaceutically acceptable derivative thereof, wherein:
a is 0-2;
R¹ is H, alkyl, haloalkyl or halo;
R² is alkyl, cycloalkyl or haloalkyl;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is CR²²R²³, where R²² and R²³ are each independently H or alkyl;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷; and
R¹⁷ is alkoxy, alkenyloxy, alkynyloxy or —O—(CH₂)₂—R¹⁹; where R¹⁹ is —OR⁹ or —NR⁹R¹¹.

In another embodiment, the compounds for use in the compositions and methods provided herein have Formula Ia:

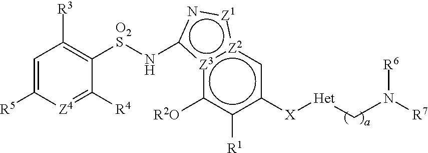

or a pharmaceutically acceptable derivative thereof, wherein:
a is 0-2;
R¹ is H, alkyl, haloalkyl or halo;
R² is alkyl, cycloalkyl or haloalkyl;
R³ is —O—(CH₂)_{b}—R¹;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is CR²²R²³, where R²² and R²³ are each independently H or alkyl;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷; and
R¹⁷ is alkoxy, alkenyloxy, alkynyloxy or —O—(CH₂)₂—R¹⁹; where R¹⁹ is —OR⁹ or —NR⁹R¹¹.

In another embodiment, the compounds for use in the compositions and methods provided herein have Formula I or Ia, wherein:
R¹ is H;
R² is alkyl, cycloalkyl or haloalkyl;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
X is CH₂; and
R¹⁷ is alkoxy.

In another embodiment, the compounds for use in the compositions and methods provided herein have Formula I or Ia, wherein:
R¹ is H;
R² is alkyl or haloalkyl;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
X is CH₂; and
R¹⁷ is alkoxy.

In one embodiment, provided is a compound of Formula II for use in the compositions and methods provided herein:

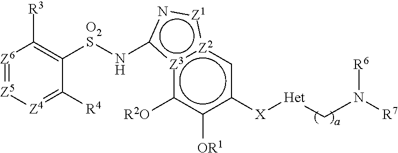

or a pharmaceutically acceptable derivative thereof, wherein:
a is 0-2;
R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR²OR²¹.

In one embodiment, provided is a compound of Formula IIa for use in the compositions and methods provided herein:

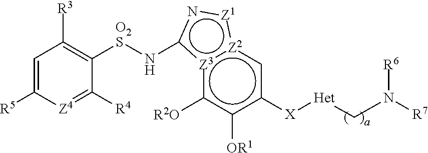

or a pharmaceutically acceptable derivative thereof, wherein:
a is 0-2;
R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR²OR²¹.

In another embodiment, the compounds for use in the compositions and methods provided herein have Formula II or IIa, wherein:
R⁸ is H, CN, OH, alkoxy, aryl, heteroaryl, NR⁹R¹⁰ or C(O)R¹¹;
R⁹ is H and R¹⁰ is H or C(O)R¹³; where R¹³ is alkyl or alkoxy; and
R¹¹ is alkyl or alkoxy;
R⁵ and R¹⁶ are selected from (i) or (ii):
(i) R⁵ and R¹⁶ are each independently H, cyano, halo, alkoxy, alkyl, alkenyl or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula II or IIa, wherein:
R⁸ is H, CN, OH, alkoxy, aryl, NR⁹R¹¹ or C(O)R¹¹;
R⁹ is H and R¹⁰ is C(O)R¹³; where R¹³ is alkyl; and
R¹¹ is alkoxy;
R⁵ and R¹⁶ are selected from (i) or (ii):
(i) R⁵ is H, cyano, alkoxy or alkyl, and R¹⁶ is H, alkyl, alkoxy, halo or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula II or IIa, wherein R¹ and R² together form C₂₋₄alkylene.

In another embodiment, the compounds for use in the compositions and methods provided herein have Formula II or IIa, wherein R¹ and R² together form propylene.

In another embodiment, the compounds for use in the compositions and methods provided herein have Formula II:

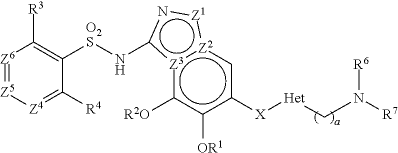

or a pharmaceutically acceptable derivative thereof, wherein:
a is 0-2;
R¹ and R² are selected from (i) or (ii);
(i) R¹ is alkyl, cycloalkyl or haloalkyl; and R² is H, alkyl, haloalkyl, alkoxy, cycloalkoxy, haloalkoxy or halo, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is CR¹⁶;
Z⁵ is CR⁵;
Z⁶ is N or CH;
R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula IIa:

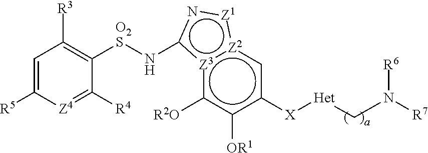

or a pharmaceutically acceptable derivative thereof, wherein:
a is 0-2;
R¹ and R² are selected from (i) or (ii):
(i) R¹ is alkyl, cycloalkyl or haloalkyl; and R² is H, alkyl, haloalkyl, alkoxy, cycloalkoxy, haloalkoxy or halo; or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is CR¹⁶;
R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula II or IIa, wherein:
R¹ and R² are selected from (i) or (ii):
(i) R¹ is alkyl, cycloalkyl or haloalkyl, and R² is H or alkoxy, or
(ii) R¹ and R² together form alkylene;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula II or IIa, wherein:
R¹ and R² are selected from (i) or (ii):
(i) R¹ is alkyl or haloalkyl, and R² is H or alkoxy, or
(ii) R¹ and R² together form alkylene;
R⁸ is H, CN, OH, aryl, NR⁹R¹⁰ or C(O)R¹;
R⁹ and R¹⁰ are each independently H or C(O)R¹³; where R¹³ is alkyl;
R¹¹ is alkoxy;
R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula II or IIa, wherein R⁵ and R¹⁶ together form C₃₋₅alkylene.

In another embodiment, the compounds for use in the compositions and methods provided herein have Formula II or IIa, wherein R⁵ and R¹⁶ together form propylene.

In another embodiment, the compounds for use in the compositions and methods provided herein have Formula II or IIa, wherein X is O or S(O)ₓ, where x is 0-2. In another embodiment, the compounds for use in the compositions and methods provided herein have Formula II or IIa, wherein X is O or S.

In another embodiment, the compounds for use in the compositions and methods provided herein have Formula II:

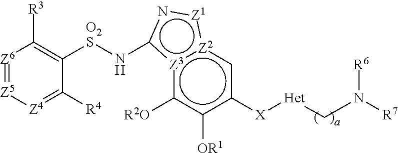

or a pharmaceutically acceptable derivative thereof, wherein:
a is 0-2;
R¹ and R² are selected from (i) or (ii):
(i) R¹ is alkyl, cycloalkyl or haloalkyl, and R² is H, alkyl, haloalkyl, alkoxy, cycloalkoxy, haloalkoxy or halo, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O or S(O)ₓ, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula Ha:

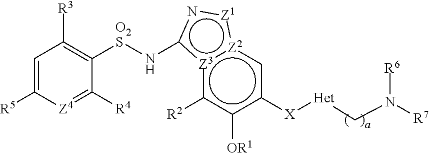

or a pharmaceutically acceptable derivative thereof, wherein:
a is 0-2;
R¹ and R² are selected from (i) or (ii):
(i) R¹ is alkyl, cycloalkyl or haloalkyl, and R² is H, alkyl, haloalkyl, alkoxy, cycloalkoxy, haloalkoxy or halo, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is O or S(O)ₓ, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula II or IIa, wherein:
R¹ and R² are selected from (i) or (ii):
(i) R¹ is alkyl, cycloalkyl or haloalkyl, and R² is H or alkoxy, or
(ii) R¹ and R² together form alkylene;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
R⁵ and R¹⁶ are selected from (i) or (ii):
(i) R⁵ and R¹⁶ are each independently H, cyano, halo, alkoxy, alkyl, alkenyl or alkynyl, or
(ii) R⁵ and R¹⁶ together form alkylene;
X is O;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula II or IIa, wherein:
R¹ and R² are selected from (i) or (ii):
(i) R¹ is alkyl or haloalkyl, and R² is H or alkoxy, or
(ii) R¹ and R² together form alkylene;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
R⁵ and R¹⁶ are selected from (i) or (ii):
(i) R⁵ is H, cyano, alkoxy or alkyl, and R¹⁶ is H, alkyl, alkoxy, halo or alkynyl, or
(ii) R⁵ and R¹⁶ together form alkylene;
X is O;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula IIa:

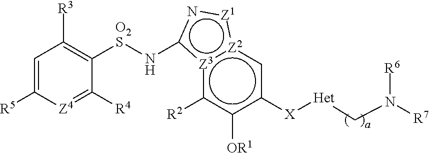

or a pharmaceutically acceptable derivative thereof, wherein:
a is 0-2;
R¹ and R² are selected from (i) or (ii):
(i) R¹ is alkyl, cycloalkyl or haloalkyl, and R² is H, alkyl, haloalkyl, alkoxy, cycloalkoxy, haloalkoxy or halo, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶, where R¹⁶ is H, alkyl, haloalkyl, alkoxy, halo, alkenyl or alkynyl;
R⁵ is H, alkyl, haloalkyl, alkoxy, halo, alkenyl or alkynyl;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula II or IIa, wherein:
R¹ and R² are selected from (i) or (ii):
(i) R¹ is alkyl, cycloalkyl or haloalkyl, and R² is H or alkoxy, or
(ii) R¹ and R² together form alkylene;
R⁵ is alkenyl or alkynyl;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula II or IIa, wherein:
R¹ and R² are selected from (i) or (ii):
(i) R¹ is alkyl or haloalkyl, and R² is H or alkoxy, or
(ii) R¹ and R² together form alkylene;
R⁵ is alkynyl;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I:

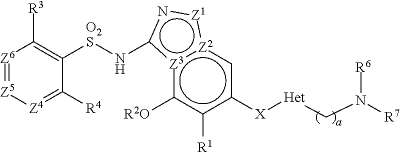

or a pharmaceutically acceptable derivative thereof, wherein:
a is 0-2;
R¹ is H, alkyl, haloalkyl, alkoxy, cycloalkoxy, haloalkoxy or halo;
R² and R⁴ together form heteroalkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²)
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula Ia:

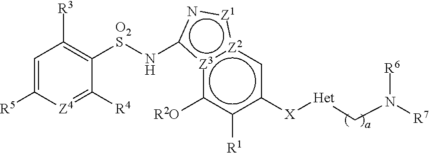

or a pharmaceutically acceptable derivative thereof, wherein:
a is 0-2;
R¹ is H, alkyl, haloalkyl, alkoxy, cycloalkoxy, haloalkoxy or halo;
R² and R⁴ together form heteroalkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula I or Ia, wherein:
R¹ is H or alkoxy;
R² and R⁴ together form —O—(CH₂)ᵣ or —O—(CH₂)₁₋₃—O—(CH₂)₁₋₃—, where r is an integer from 2 to 5 and the terminal O is attached to the R⁴ position of Formula I;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
R⁵ and R¹⁶ are selected from (i) or (ii):
(i) R⁵ and R¹⁶ are each independently H, cyano, halo, alkoxy, alkyl, alkenyl or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I or Ia, wherein:
R¹ is H or alkoxy;
R² and R⁴ together form —O—(CH₂)ᵣ or —O—(CH₂)₁₋₃—O—(CH₂)₁₋₃—, where r is an integer from 2 to 5 and the terminal 0 is attached to the R⁴ position of Formula I;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
R⁵ and R¹⁶ are selected from (i) or (ii):
(i) R⁵ is H, cyano, alkoxy or alkyl, and R¹⁶ is H, alkyl, alkoxy, halo or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula II:

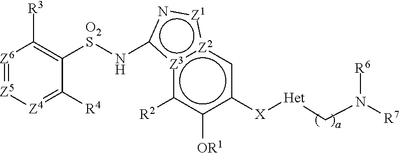

or a pharmaceutically acceptable derivative thereof, wherein:
a is 0-2;
R¹ is alkyl, cycloalkyl or haloalkyl;
R² is H, alkyl, haloalkyl, alkoxy, cycloalkoxy, haloalkoxy or halo;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹¹ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is CR²²R²³, where R²² and R²³ are each independently H or alkyl;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷; and
R¹⁷ is alkoxy, alkenyloxy, alkynyloxy or —O—(CH₂)₂—R¹⁹; where R¹⁹ is —OR⁹ or —NR⁹R¹¹.

In another embodiment, the compounds for use in the compositions and methods provided herein have Formula IIa:

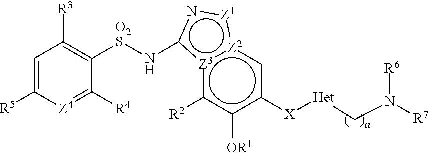

or a pharmaceutically acceptable derivative thereof, wherein:
a is 0-2;
R¹ is alkyl, cycloalkyl or haloalkyl;
R² is H, alkyl, haloalkyl, alkoxy, cycloalkoxy, haloalkoxy or halo;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is CR²²R²³, where R²² and R²³ are each independently H or alkyl;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷; and
R¹⁷ is alkoxy, alkenyloxy, alkynyloxy or —O—(CH₂)₂—R¹⁹; where R¹⁹ is —OR⁹ or —NR⁹R¹¹.

In another embodiment, the compounds for use in the compositions and methods provided herein have Formula II or IIa, wherein:
R¹ is alkyl, cycloalkyl or haloalkyl;
R² is H or alkoxy;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
X is CH₂; and
R¹⁷ is alkoxy.

In another embodiment, the compounds for use in the compositions and methods provided herein have Formula II or IIa, wherein:
R¹ is alkyl or haloalkyl;
R² is H or alkoxy;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
X is CH₂; and
R¹⁷ is alkoxy.

In one embodiment, provided is a compound of Formula III for use in the compositions and methods provided herein:

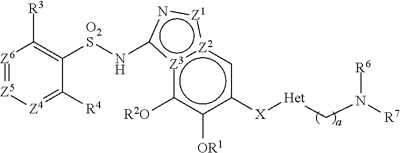

or a pharmaceutically acceptable derivative thereof, wherein:
a is 0-2;
R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR²OR²¹.

In one embodiment, provided is a compound of Formula IIIa for use in the compositions and methods provided herein:

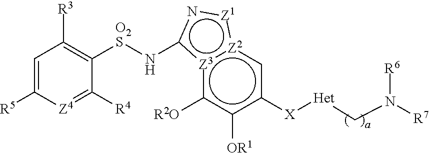

or a pharmaceutically acceptable derivative thereof, wherein:
a is 0-2;
R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹¹;
R¹¹ is hydroxy, alkyl, alkoxy or NR¹⁴R¹⁵;
R¹² is alkyl or NR¹⁴R¹¹;
R¹⁴ and R¹¹ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR²OR²¹.

In another embodiment, the compounds for use in the compositions and methods provided herein have Formula III or IIIa, wherein:
R⁸ is H, CN, OH, alkoxy, aryl, heteroaryl, NR⁹R¹⁰ or C(O)R¹¹;
R⁹ is H and R¹⁰ is H or C(O)R¹³; where R¹³ is alkyl or alkoxy; and
R¹¹ is alkyl or alkoxy;
R⁵ and R¹⁶ are selected from (i) or (ii):
(i) R⁵ and R¹⁶ are each independently H, cyano, halo, alkoxy, alkyl, alkenyl or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula III or IIIa, wherein:
R⁸ is H, CN, OH, alkoxy, aryl, NR⁹R¹⁰ or C(O)R¹¹;
R⁹ is H and R¹⁰ is C(O)R¹³; where R¹³ is alkyl; and
R¹¹ is alkoxy;
R⁵ and R¹⁶ are selected from (i) or (ii):
(i) R⁵ is H, cyano, alkoxy or alkyl, and R¹⁶ is H, alkyl, alkoxy, halo or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula III or IIIa, wherein R¹ and R² together form C₁₋₄alkylene optionally substituted with one or more halo or methyl.

In another embodiment, the compounds for use in the compositions and methods provided herein have Formula III or IIIa, wherein R¹ and R² together form methylene or ethylene each optionally substituted with one or two halo or methyl. In another embodiment, the compounds for use in the compositions and methods provided herein have Formula III or IIIa, wherein R¹ and R² together form unsubstituted methylene or ethylene.

In another embodiment, the compounds for use in the compositions and methods provided herein have Formula III:

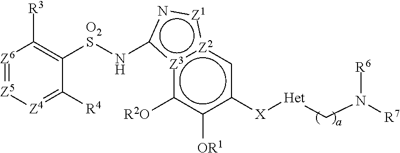

or a pharmaceutically acceptable derivative thereof, wherein:
a is 0-2;
R¹ and R² are selected from (i) or (ii):
(i) R¹ and R² are each independently alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹¹ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is CR¹⁶;
Z⁵ is CR⁵;
Z⁶ is N or CH;
R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula IIIa:

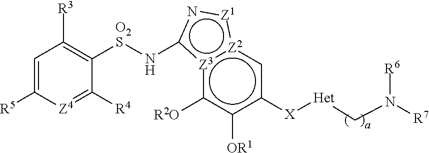

or a pharmaceutically acceptable derivative thereof, wherein:
a is 0-2;
R¹ and R² are selected from (i) or (ii):
(i) R¹ and R² are each independently alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²)
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is CR¹⁶;
R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula III or IIIa, wherein:
R¹ and R² are selected from (i) or (ii):
(i) R¹ and R² are each alkyl, or
(ii) R¹ and R² together form alkylene optionally substituted with one or more halo or methyl;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula III or IIIa, wherein R⁵ and R¹⁶ together form C₃₋₅alkylene.

In another embodiment, the compounds for use in the compositions and methods provided herein have Formula III or IIIa, wherein R⁵ and R¹⁶ together form propylene.

In another embodiment, the compounds for use in the compositions and methods provided herein have Formula III or IIIa, wherein X is O or S(O)ₓ, where x is 0-2. In another embodiment, the compounds for use in the compositions and methods provided herein have Formula III or IIIa, wherein X is O or S.

In another embodiment, the compounds for use in the compositions and methods provided herein have Formula III:

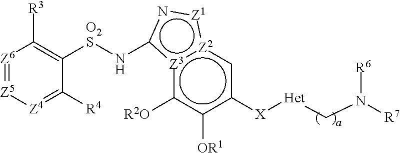

or a pharmaceutically acceptable derivative thereof, wherein:
a is 0-2;
R¹ and R² are selected from (i) or (ii):
(i) R¹ and R² are each independently alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O or S(O)ₓ, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula IIIa:

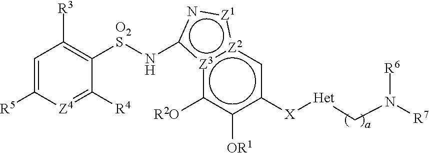

or a pharmaceutically acceptable derivative thereof, wherein:
a is 0-2;
R¹ and R² are selected from (i) or (ii):
(i) R¹ and R² are each independently alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is O or S(O)ₓ, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula III or IIIa, wherein:
R¹ and R² are selected from (i) or (ii):
(i) R¹ and R² are each alkyl, or
(ii) R¹ and R² together form alkylene optionally substituted with one or more halo or methyl;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
R⁵ and R¹⁶ are selected from (i) or (ii):
(i) R⁵ and R¹⁶ are each independently H, cyano, halo, alkoxy, alkyl, alkenyl or alkynyl, or
(ii) R⁵ and R¹⁶ together form alkylene;
X is O;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula III or IIIa, wherein:
R¹ and R² are selected from (i) or (ii):
(i) R¹ and R² are each alkyl, or
(ii) R¹ and R² together form alkylene optionally substituted with one or more halo or methyl;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
R⁵ and R¹⁶ are selected from (i) or (ii):
(i) R⁵ is H, cyano, alkoxy or alkyl, and R¹⁶ is H, alkyl, alkoxy, halo or alkynyl, or
(ii) R⁵ and R¹⁶ together form alkylene;
X is O;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula IIIa:

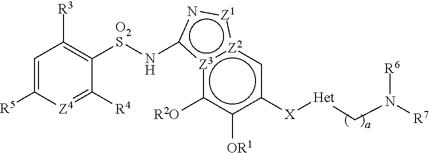

or a pharmaceutically acceptable derivative thereof, wherein:
a is 0-2;
R¹ and R² are selected from (i) or (ii):
(i) R¹ and R² are each independently alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶, where R¹⁶ is H, alkyl, haloalkyl, alkoxy, halo, alkenyl or alkynyl;
R⁵ is H, alkyl, haloalkyl, alkoxy, halo, alkenyl or alkynyl;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula III or IIIa, wherein:
R¹ and R² are selected from (i) or (ii):
(i) R¹ and R² are each independently alkyl, cycloalkyl or haloalkyl, or
(ii) R¹ and R² together form alkylene optionally substituted with one or more halo or methyl;
R⁵ is alkenyl or alkynyl;
R⁸ is H, CN, OH, aryl, heteroaryl, NR⁹R¹⁰ or C(O)R¹¹;
R⁹ and R¹⁰ are each independently H or C(O)R¹³; where R¹³ is alkyl or alkoxy;
R¹¹ is alkyl or alkoxy;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula III or IIIa, wherein:
R¹ and R² are selected from (i) or (ii):
(i) R¹ and R² are each independently alkyl or haloalkyl, or
(ii) R¹ and R² together form alkylene optionally substituted with one or more halo or methyl;
R⁵ is alkynyl;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula III:

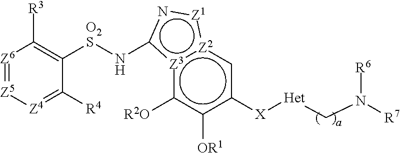

or a pharmaceutically acceptable derivative thereof, wherein:
a is 0-2;
R¹ is alkyl or haloalkyl;
R² and R⁴ together form heteroalkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula IIIa:

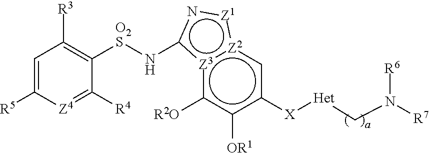

or a pharmaceutically acceptable derivative thereof, wherein:
a is 0-2;
R¹ is alkyl or haloalkyl;
R² and R⁴ together form heteroalkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is N or CR¹⁶;
R⁵ and R¹⁶ are selected from (i) or (ii):
(i) R⁵ and R¹⁶ are each independently H, cyano, halo, alkoxy, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

In another embodiment, the compounds for use in the compositions and methods provided herein have Formula III or IIIa, wherein:
R¹ is alkyl;
R² and R⁴ together form —O—(CH₂)ᵣ or —O—(CH₂)₁₋₃—O—(CH₂)₁₋₃—, where r is an integer from 2 to 5 and the terminal 0 is attached to the R⁴ position of Formula III or IIIa;
R⁸ is H, CN, OH, aryl, heteroaryl, NR⁹R¹⁰ or C(O)R¹;
R⁹ and R¹⁰ are each independently H or C(O)R¹³; where R¹³ is alkyl or alkoxy;
R¹¹ is alkyl or alkoxy;
R⁵ and R¹⁶ are selected from (i) or (ii):
(i) R⁵ and R¹⁶ are each independently H, cyano, halo, alkoxy, alkyl, alkenyl or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula III or IIIa, wherein:
R¹ is alkyl;
R² and R⁴ together form —O—(CH₂)ᵣ or —O—(CH₂)₁₋₃—O—(CH₂)₁₋₃—, where r is an integer from 2 to 5 and the terminal 0 is attached to the R⁴ position of Formula III or IIIa;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
R⁵ and R¹⁶ are selected from (i) or (ii):
(i) R⁵ is H, cyano, alkoxy or alkyl, and R¹⁶ is H, alkyl, alkoxy, halo or alkynyl, or
(ii) R⁵ and R¹⁶ together form alkylene;
X is O or CH₂;
R¹⁷ is alkyl, haloalkyl, alkoxy, alkoxyalkyl, aminoalkyl, alkenyl, haloalkenyl, alkynyl or hydroxymethylalkynyl; and
R¹⁸ is alkyl, haloalkyl, alkoxyalkyl, aminoalkyl, alkenyl, haloalkenyl, alkynyl or hydroxymethylalkynyl.

In another embodiment, the compounds for use in the compositions and methods provided herein have Formula I, Ia, II, IIa, III or IIIa, wherein X is O. In another embodiment, the compounds for use in the compositions and methods provided herein have Formula I, Ia, II, IIa, III or IIIa, wherein X is CH₂.

In another embodiment, a is 0. In another embodiment, a is 1. In another embodiment, a is 2. In another embodiment, a is 0 or 1.

In another embodiment, the compounds for use in the compositions and methods provided herein have Formula I or Ia, wherein R² is alkyl. In another embodiment, the compounds for use in the compositions and methods provided herein have Formula I or Ia, wherein R² is methyl. In another embodiment, the compounds for use in the compositions and methods provided herein have Formula I or Ia, wherein R² is haloalkyl. In another embodiment, the compounds for use in the compositions and methods provided herein have Formula I or Ia, wherein R² is fluoromethyl, difluoromethyl or trifluoromethyl. In another embodiment, the compounds for use in the compositions and methods provided herein have Formula I or Ia, wherein R² is difluoromethyl.

In another embodiment, the compounds for use in the compositions and methods provided herein have Formula II or IIa, wherein R² is H. In another embodiment, the compounds for use in the compositions and methods provided herein have Formula II or IIa, wherein R² is alkoxy. In another embodiment, the compounds for use in the compositions and methods provided herein have Formula II or IIa, wherein R² is methoxy. In another embodiment, the compounds for use in the compositions and methods provided herein have Formula II or IIa, wherein R² is haloalkoxy. In another embodiment, the compounds for use in the compositions and methods provided herein have Formula II or IIa, wherein R² is fluoromethoxy, difluoromethoxy or trifluoromethoxy. In another embodiment, the compounds for use in the compositions and methods provided herein have Formula II or IIa, wherein R² is difluoromethoxy.

In another embodiment, b is 1. In another embodiment, b is 2. In another embodiment, b is 3. In another embodiment, b is 4. In another embodiment, b is 1 or 2.

In another embodiment, the compounds for use in the compositions and methods provided herein have Formula I, Ia, II, IIa, III or IIIa, wherein R⁸ is H, phenyl, 4-fluorophenyl, morpholinyl, COOH, COOMe, C(═NH)NHOH, tetrazolyl, oxadiazolyl, triazolyl, pyridyl, imidazolyl, oxazolidinyl, NHC(O)OMe, cyano, hydroxy, amino or acetylamino. In another embodiment, the compounds for use in the compositions and methods provided herein have Formula I, Ia, II, IIa, III or IIIa, wherein R⁸ is H, phenyl, COOMe, hydroxy, cyano, amino or acetylamino. In another embodiment, the compounds for use in the compositions and methods provided herein have Formula I, Ia, II, IIa, III or IIIa, wherein R⁸ is hydrogen. In another embodiment, the compounds for use in the compositions and methods provided herein have Formula I, Ia, II, IIa, III or IIIa, wherein b is 1 and R⁸ is hydrogen.

In another embodiment, the compounds for use in the compositions and methods provided herein have Formula I, Ia, II, IIa, III or IIIa, wherein R⁴ is alkoxy. In another embodiment, the compounds for use in the compositions and methods provided herein have Formula I, Ia, II, IIa, III or IIIa, wherein R⁴ is methoxy. In another embodiment, the compounds for use in the compositions and methods provided herein have Formula I, Ia, II, IIa, III or IIIa, wherein R⁴ is H.

In another embodiment, the compounds for use in the compositions and methods provided herein have Formula I, Ia, II, IIa, III or IIIa, wherein Z¹ is NH or O, and Z² and Z³ are C. In another embodiment, the compounds for use in the compositions and methods provided herein have Formula I, Ia, II, IIa, III or IIIa, wherein Z¹ is O, and Z² and Z³ are C.

In another embodiment, Z⁴ is N. In another embodiment, Z⁴ is CR¹⁶.

In another embodiment, the compounds for use in the compositions and methods provided herein have Formula I, Ia, II, IIa, III or IIIa, wherein R⁵ is H, cyano, alkoxy or alkyl, and R¹⁶ is H, alkyl, alkoxy, halo or alkynyl. In another embodiment, the compounds for use in the compositions and methods provided herein have Formula I, Ia, II, IIa, III or IIIa, wherein R⁵ is H, cyano, methoxy or methyl, and R¹⁶ is H, methyl, ethyl, isopropyl, tert-butyl, methoxy, chloro, fluoro, ethynyl or propynyl. In another embodiment, the compounds for use in the compositions and methods provided herein have Formula I, Ia, II, IIa, III or IIIa, wherein R⁵ is H, cyano, methoxy or methyl, and R¹⁶ is H, methyl, ethyl, isopropyl, tert-butyl, methoxy, chloro, ethynyl or propynyl. In another embodiment, the compounds for use in the compositions and methods provided herein have Formula I, Ia, II, IIa, III or IIIa, wherein R⁵ is H and R¹⁶ is H, methyl, ethyl, isopropyl, tert-butyl or methoxy.

In another embodiment, the compounds for use in the compositions and methods provided herein have Formula I, Ia, II, IIa, III or IIIa, wherein Het is a 5- or 6-membered heteroaryl, and R⁶ is H or alkyl. In another embodiment, the compounds for use in the compositions and methods provided herein have Formula I, Ia, II, IIa, III or IIIa, wherein Het is a 5-membered heteroaryl, and R⁶ is H or methyl. In another embodiment, the compounds for use in the compositions and methods provided herein have Formula I, Ia, II, IIa, III or IIIa, wherein Het is pyrazolyl, oxazolyl, thiazolyl or pyridyl, and R⁶ is H or methyl. In another embodiment, the compounds for use in the compositions and methods provided herein have Formula I, Ia, II, IIa, III or IIIa, wherein Het is pyrazolyl, and R⁶ is H or methyl. In another embodiment, the compounds for use in the compositions and methods provided herein have Formula I, Ia, II, IIa, III or IIIa, wherein Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system. In another embodiment, the compounds for use in the compositions and methods provided herein have Formula I, Ia, II, IIa, III or IIIa, wherein Het is a 5-membered heteroaryl, R⁶ is —(CH₂)—, and R⁶ and Het together with the atoms to which they are attached form a 5,5-bicyclic ring system. In another embodiment, the compounds for use in the compositions and methods provided herein have Formula I, Ia, II, IIa, III or IIIa, wherein Het is a pyrazolyl, R⁶ is —(CH₂)—, and R⁶ and Het together with the atoms to which they are attached form a 5,5-bicyclic ring system.

In another embodiment, the compounds for use in the compositions and methods provided herein have Formula I, Ia, II, IIa, III or IIIa, wherein R⁷ is —C(O)R¹⁷. In another embodiment, the compounds for use in the compositions and methods provided herein have Formula I, Ia, II, IIa, III or IIIa, wherein R⁷ is —S(O)R¹⁸. In another embodiment, the compounds for use in the compositions and methods provided herein have Formula I, Ia, II, IIa, III or IIIa, wherein R⁷ is —SO₂R¹⁸. In another embodiment, the compounds for use in the compositions and methods provided herein have Formula I, Ia, II, IIa, III or IIIa, wherein R¹⁷ is alkyl, alkoxy, alkenyl, haloalkenyl, alkynyl, NR²⁰R²¹ or hydroxyalkynyl. In another embodiment, the compounds for use in the compositions and methods provided herein have Formula I, Ia, II, IIa, III or IIIa, wherein R¹⁷ is alkyl, alkoxy, alkenyl, haloalkenyl, alkynyl or hydroxyalkynyl. In another embodiment, the compounds for use in the compositions and methods provided herein have Formula I, Ia, II, IIa, III or IIIa, wherein R¹⁷ is methyl, methoxy, NHMe, ethenyl, 1-fluoro-1-ethenyl, ethynyl or 1-propyn-1-yl. In another embodiment, the compounds for use in the compositions and methods provided herein have Formula I, Ia, II, IIa, III or IIIa, wherein R¹⁷ is methyl, methoxy, ethenyl, 1-fluoro-1-ethenyl, ethynyl or 1-propyn-1-yl. In another embodiment, the compounds for use in the compositions and methods provided herein have Formula I, Ia, II, IIa, III or IIIa, wherein R¹⁷ is methyl or methoxy. In another embodiment, the compounds for use in the compositions and methods provided herein have Formula I, Ia, II, IIa, III or IIIa, wherein R¹⁷ is methoxy. In another embodiment, the compounds for use in the compositions and methods provided herein have Formula I, Ia, II, IIa, III or IIIa, wherein R¹⁷ is ethenyl, 1-fluoro-1-ethenyl, ethynyl or 1-propyn-1-yl. In another embodiment, the compounds for use in the compositions and methods provided herein have Formula I, Ia, II, IIa, III or IIIa, wherein R⁸ is methyl or tert-butyl.

In another embodiment, the compounds for use in the compositions and methods provided herein are selected from:

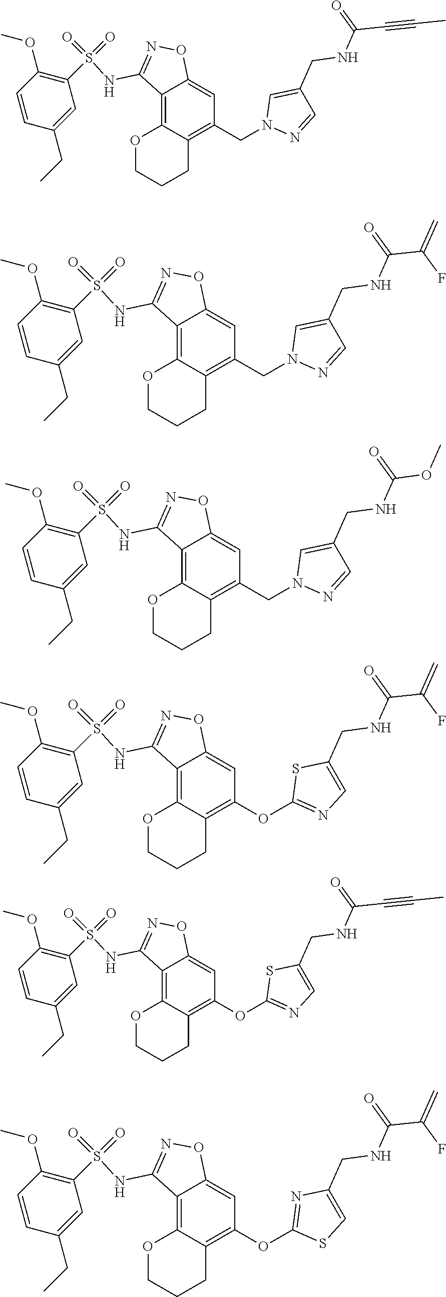

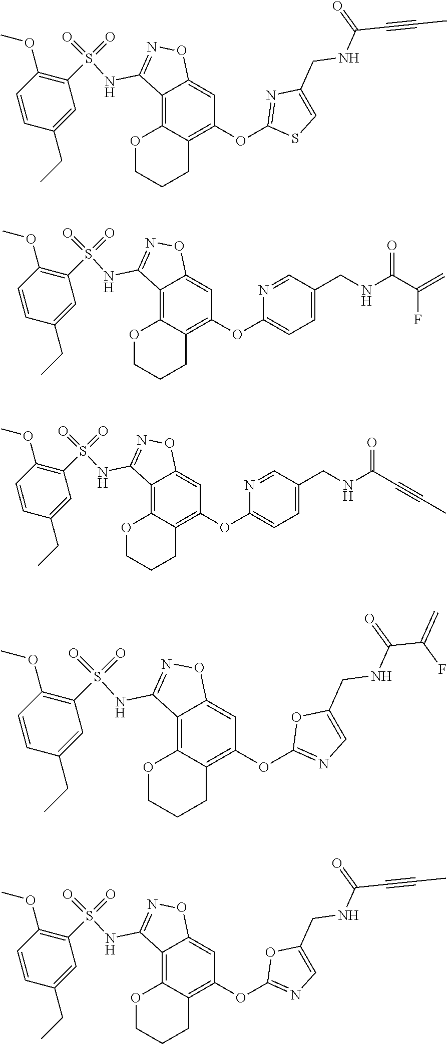

In another embodiment, the compounds for use in the compositions and methods provided herein are selected from:

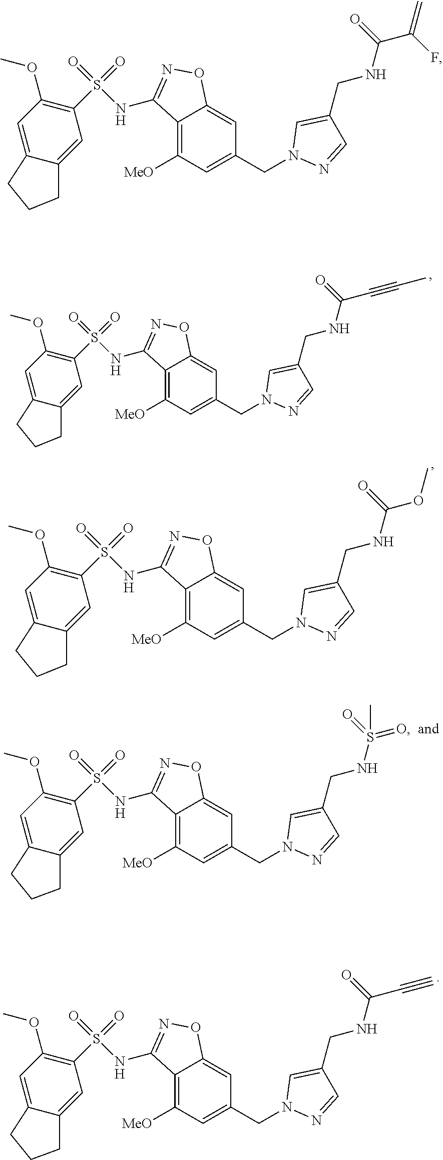

In another embodiment, the compounds for use in the compositions and methods provided herein are selected from:

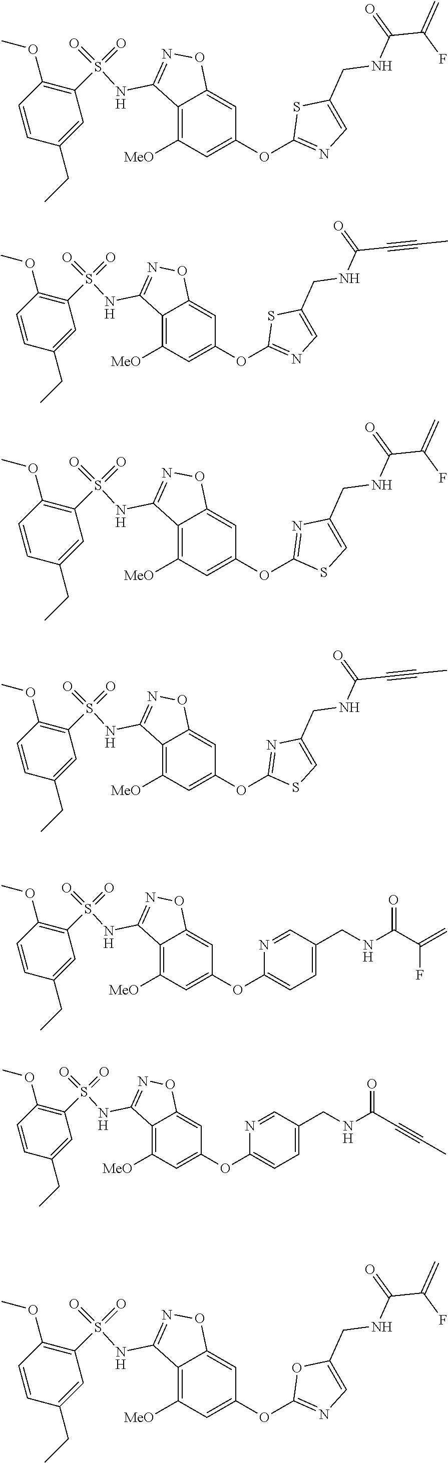

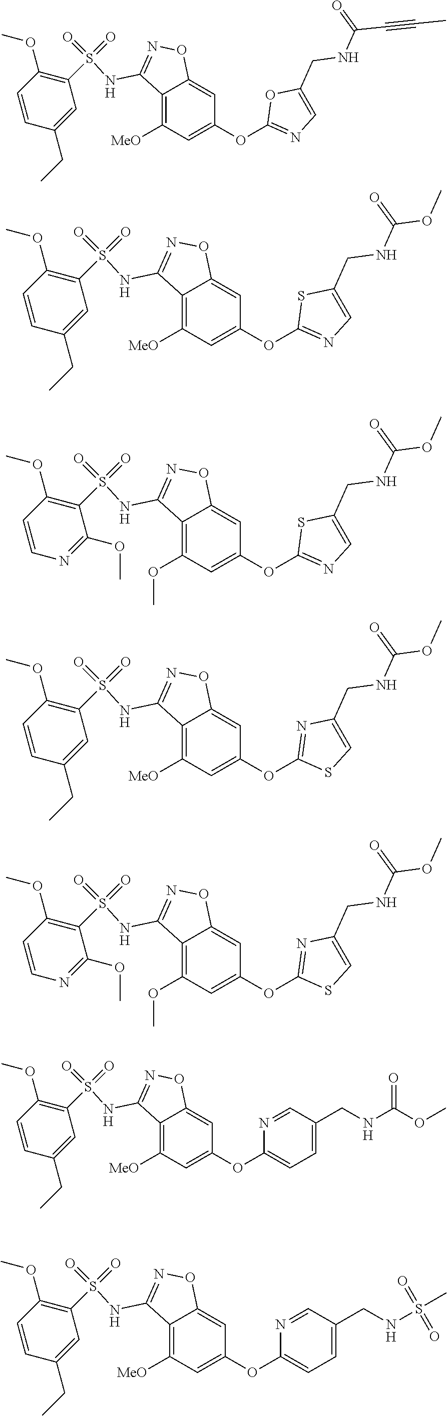

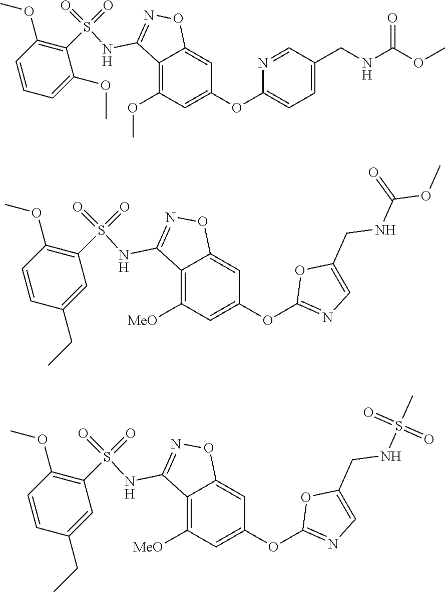

In another embodiment, the compounds for use in the compositions and methods provided herein are selected from:

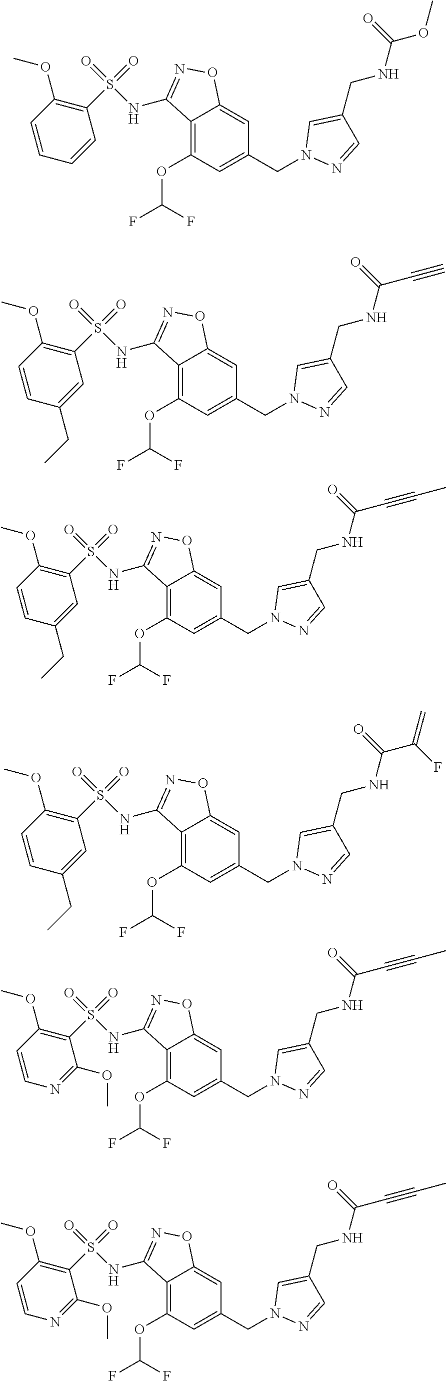

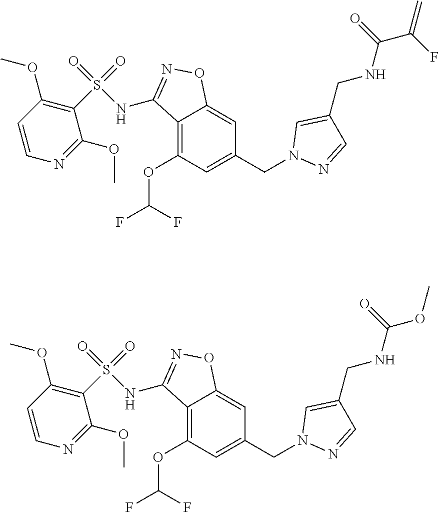

In another embodiment, the compounds for use in the compositions and methods provided herein are selected from:

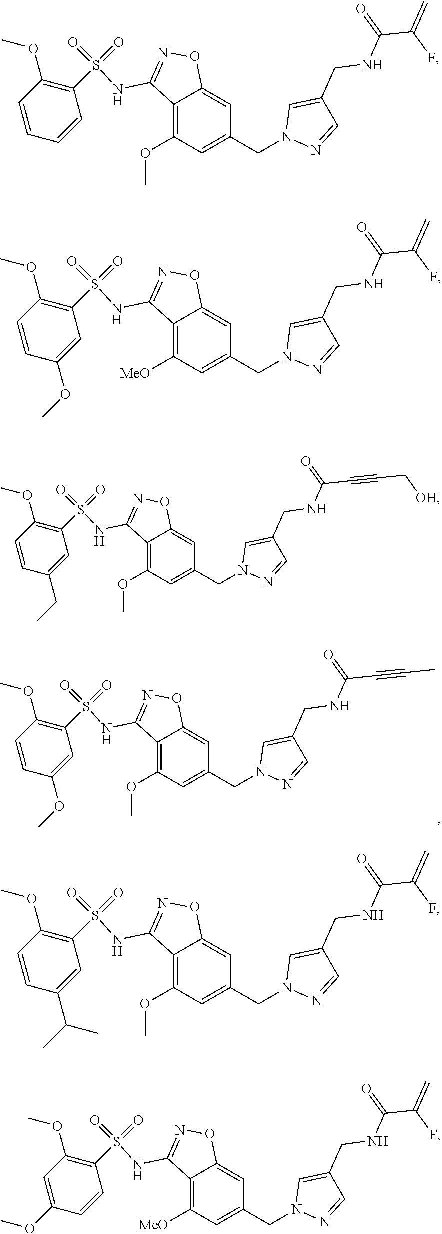

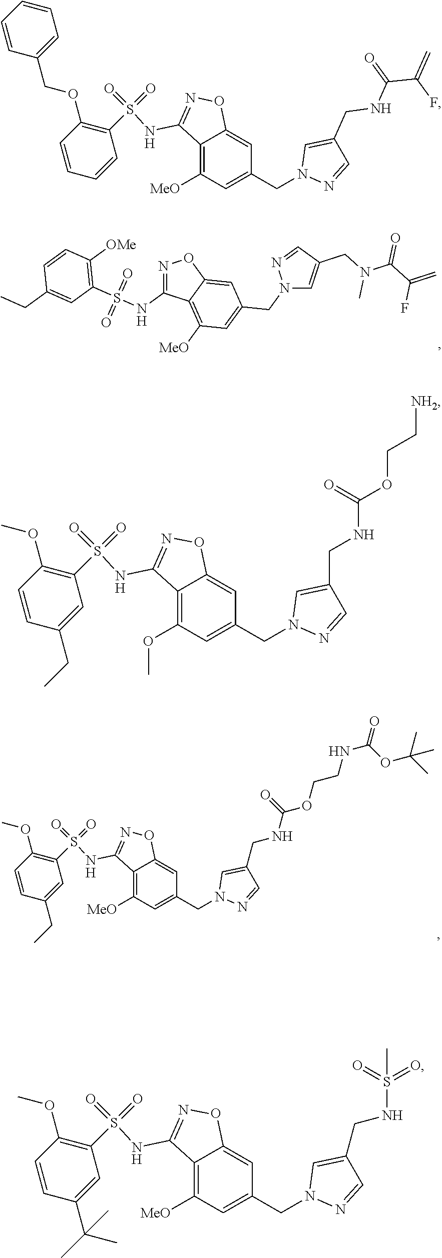

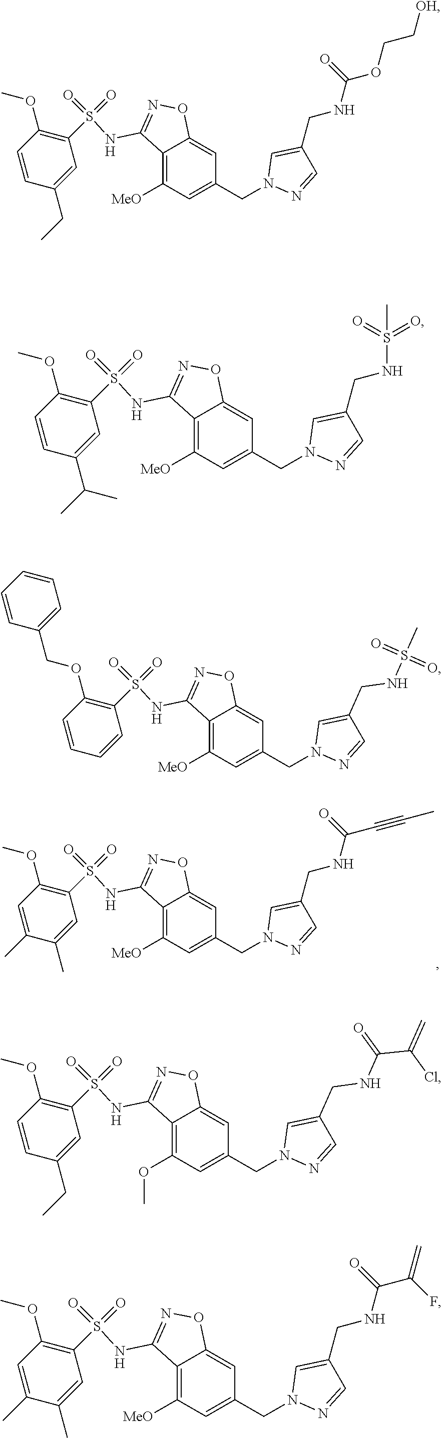

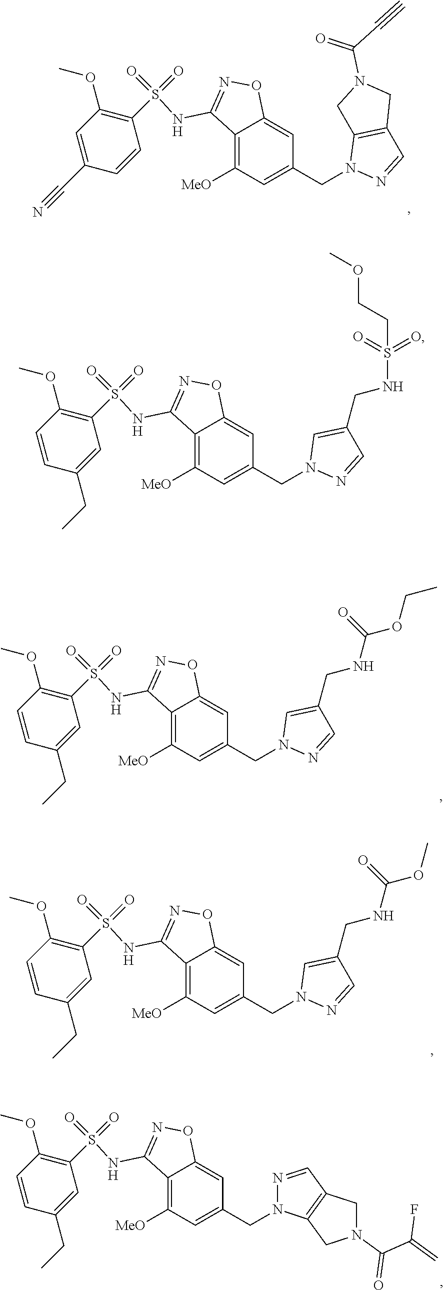

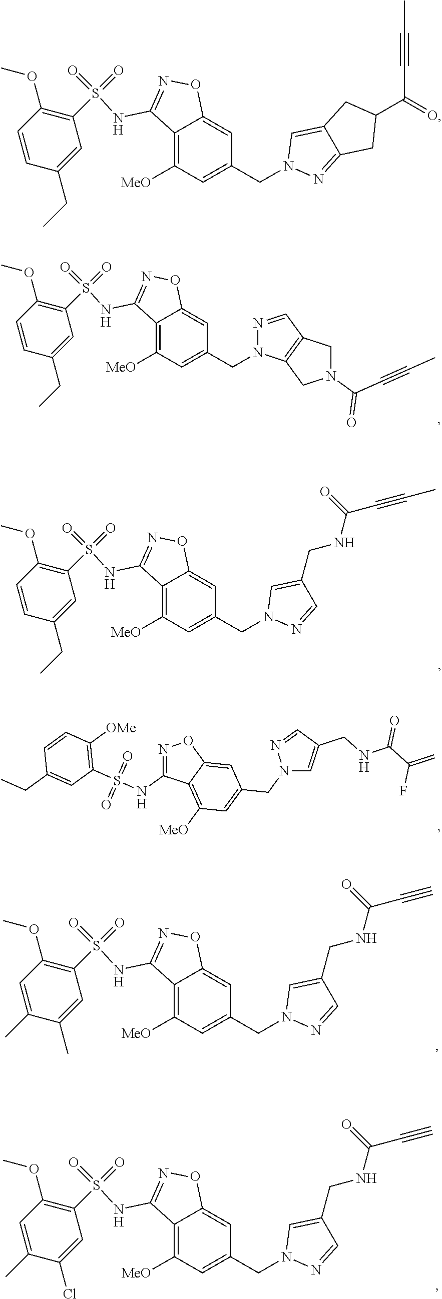

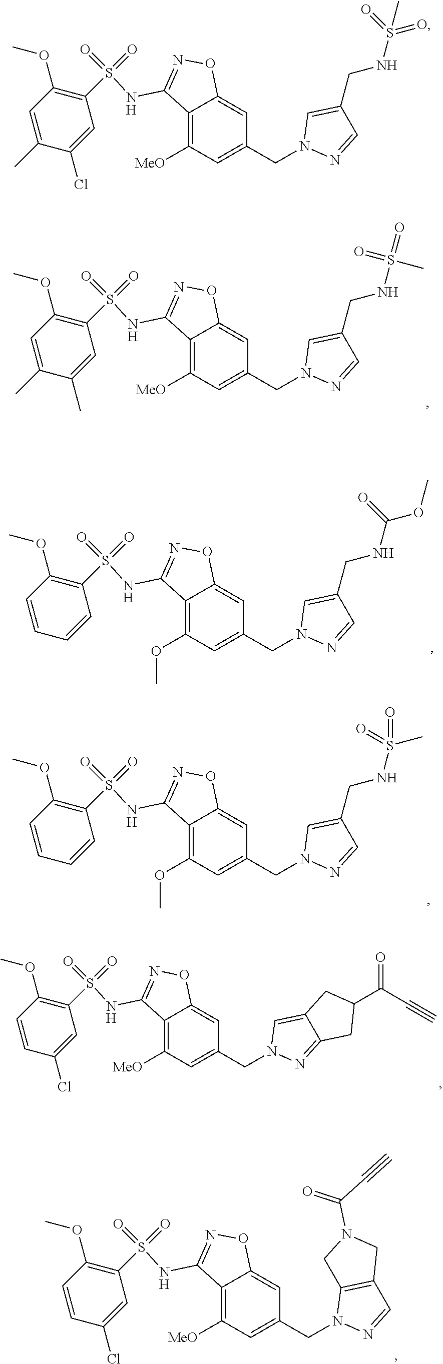

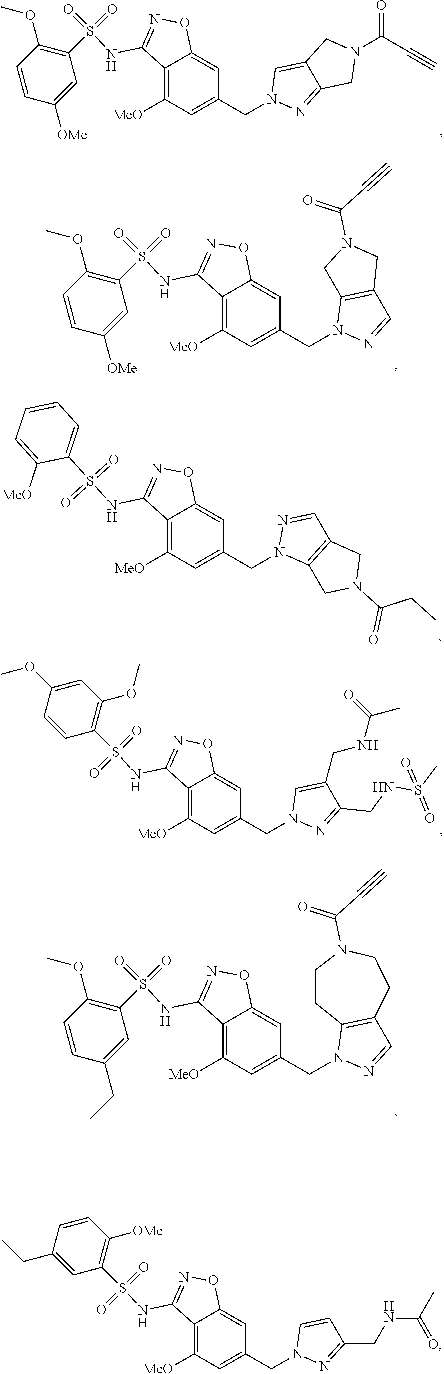

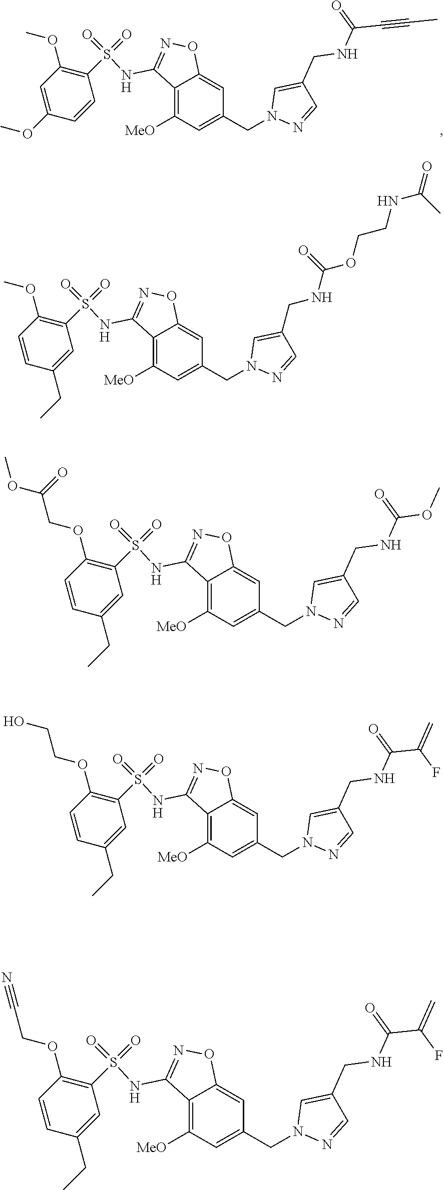

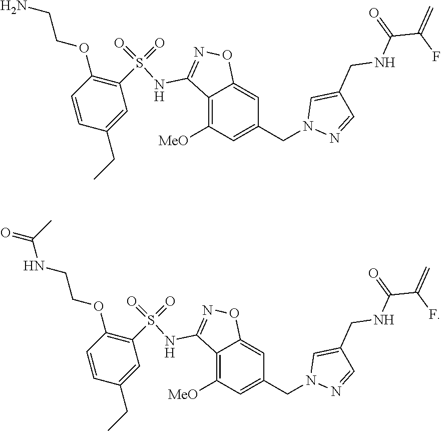 [SMILES: C=C(F)C(=O)NCc1cnn(Cc2cc(OC)c3c(NS(=O)(=O)c4cc(CC)ccc4OCCN)noc3c2)c1 ; C=C(F)C(=O)NCc1cnn(Cc2cc(OC)c3c(NS(=O)(=O)c4cc(CC)ccc4OCCNC(C)=O)noc3c2)c1]

In another embodiment, the compound for use in the compositions and methods provided herein is:

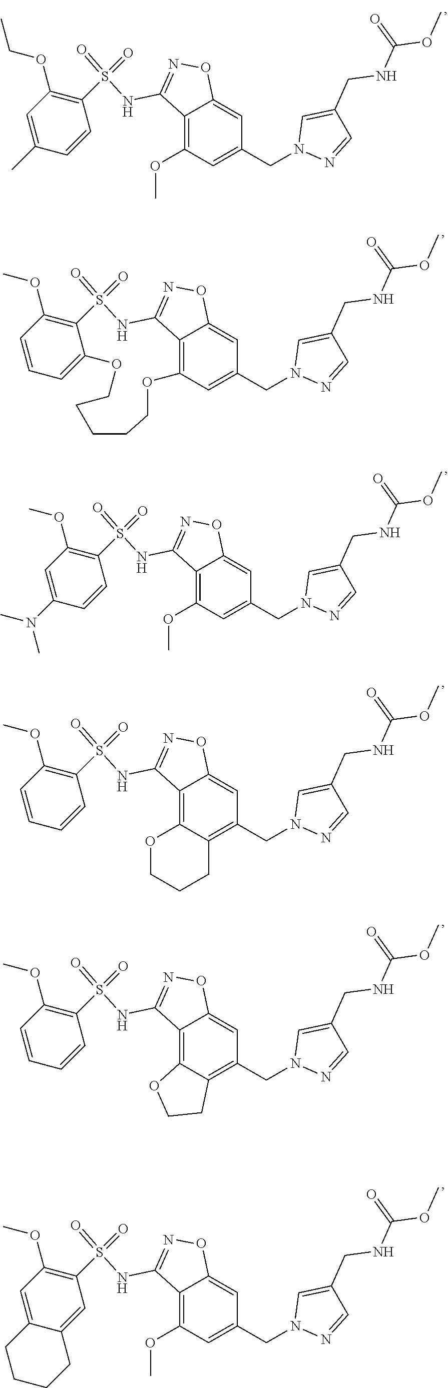

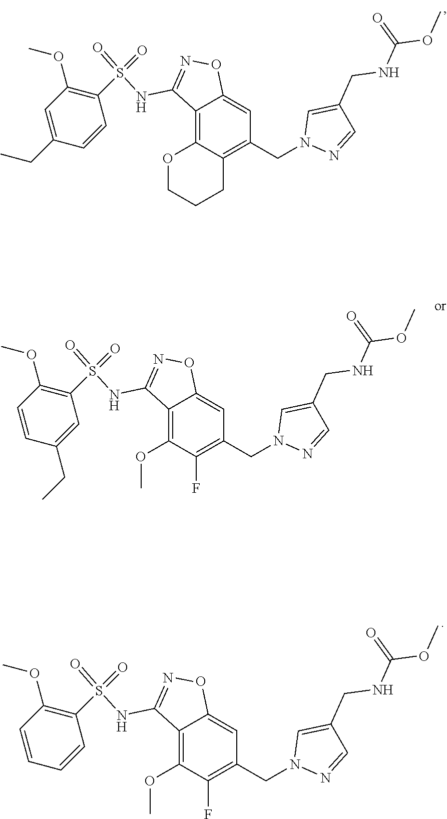

In another embodiment, the compound for use in the compositions and methods provided herein is:

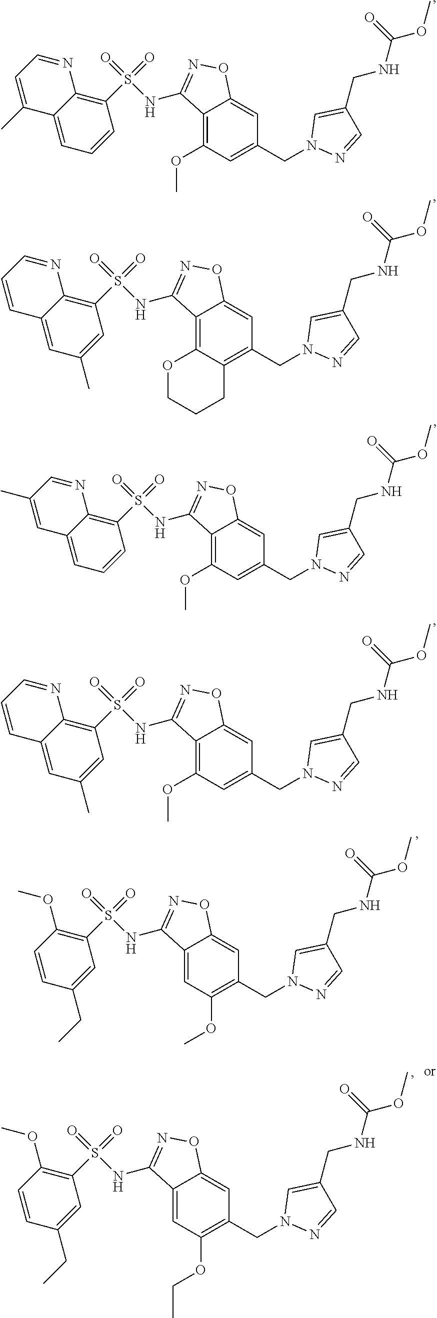

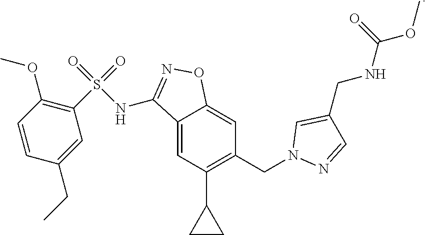

In another embodiment, the compound for use in the compositions and methods provided herein is:

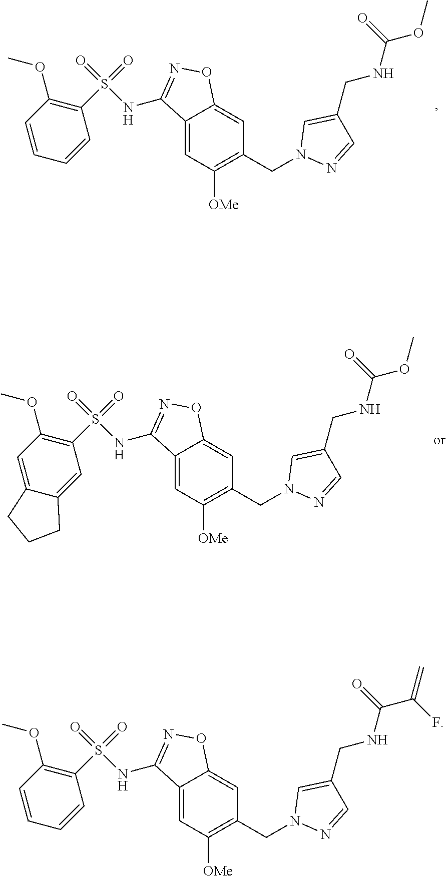

In another embodiment, the compound for use in the compositions and methods provided herein is:

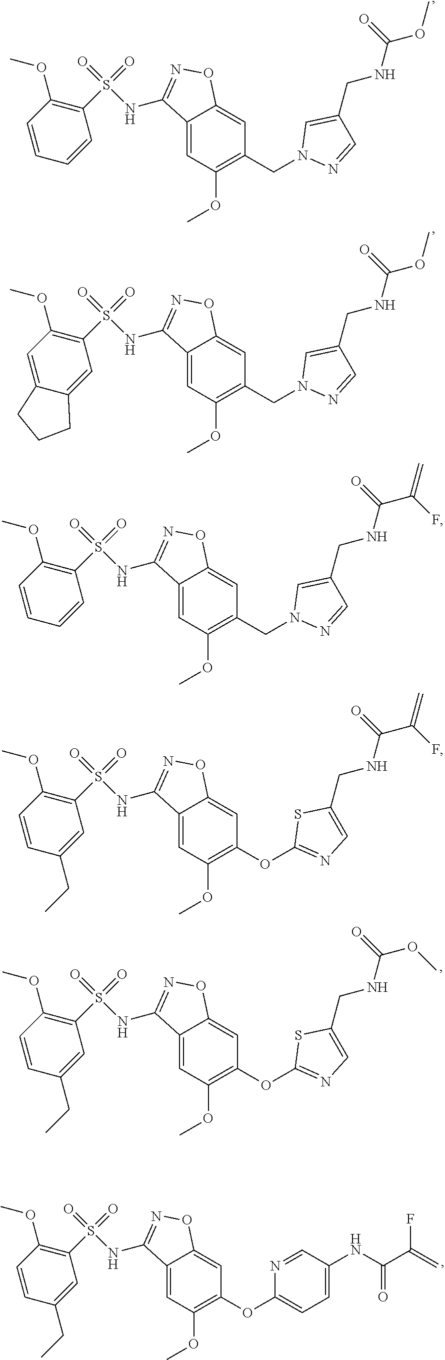

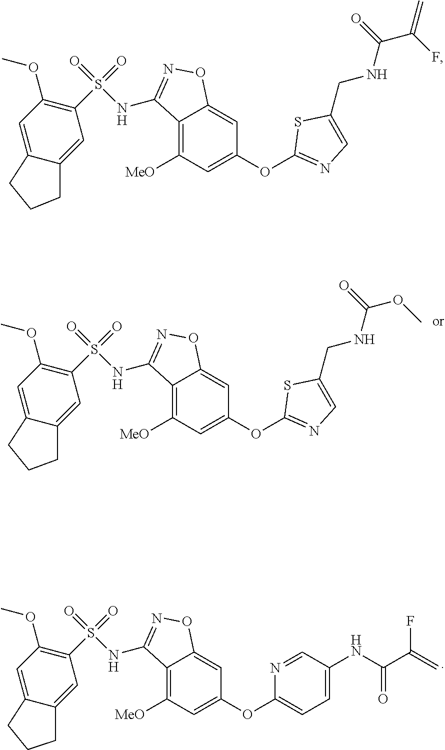

In another embodiment, the compound for use in the compositions and methods provided herein is:

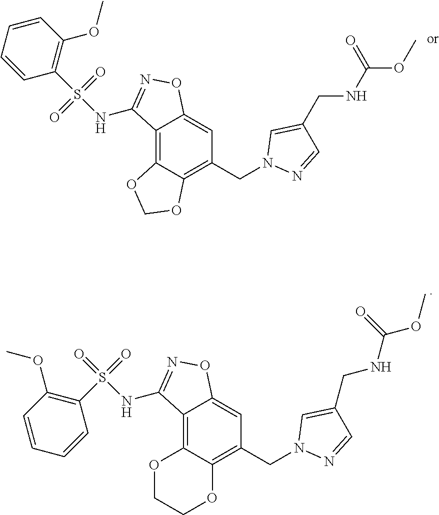

### III. Synthesis of the Compounds

The compounds provided herein can be prepared, isolated or obtained by any method apparent to those of skill in the art. Compounds provided herein can be prepared according to the Exemplary Preparation Schemes provided below. Reaction conditions, steps, and reactants not provided in the Exemplary Preparation Schemes would be apparent to, and known by, those skilled in the art.

Additional steps and reagents not provided in the Exemplary Preparation Schemes would be known to those of skill in the art. Exemplary methods of preparation are described in detail in the Examples herein.

General Scheme 1 describes the preparation of a compound of Formula I where Z¹ is O and Z² and Z³ are C; and all other groups are as defined in the Summary or in any embodiments described herein.

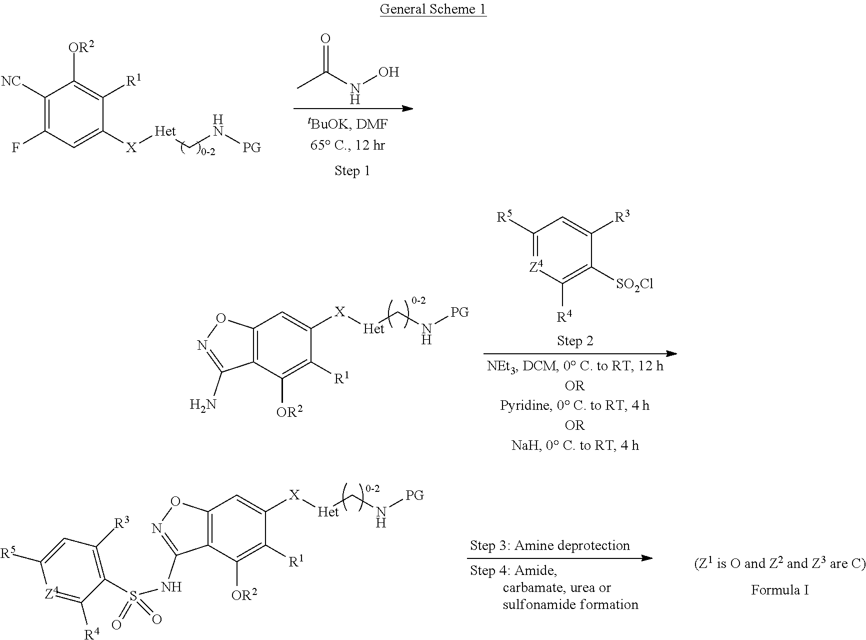

General Scheme 1 outlines a way to prepare the appropriately substituted benzo[d]isoxazole compounds provided herein where in Step 1, an appropriately substituted 2-fluorobenzonitrile is converted to a benzo[d]isoxazol-3-amine scaffold by reacting with N-hydroxyacetamide in presence of a base such as potassium t-butoxide. In Step 2, the free amine is reacted with an aryl or heteroaryl sulfonyl chloride in presence of a base such as sodium tert-pentoxide, triethylamine, pyridine or sodium hydride. This can be accomplished at 0° C., but may need to be done at room temperature or at an elevated temperature such as 60° C. The final steps involve deprotection of the amine (Step 3) followed by amide formation (either via a reaction with an appropriately substituted acid chloride or via activation of an acid using a coupling agent such as HATU, HBTU, T₃P, EDCI/HOBt or other agents known to those skilled in the art) or sulfonamide formation with a sulfonyl chloride in the presence of a base such as triethyl amine in Step 4. For preparing a carbamate or a urea derivative, an appropriately substituted carbonochloridate or isocyanate can be reacted with the free amine, in the presence of a base such as triethyl amine.

General Scheme 2 describes the preparation of a compound of Formula I where Z¹ and Z³ are N and Z² is C; and all other groups are as defined in the Summary or in any embodiments described herein.

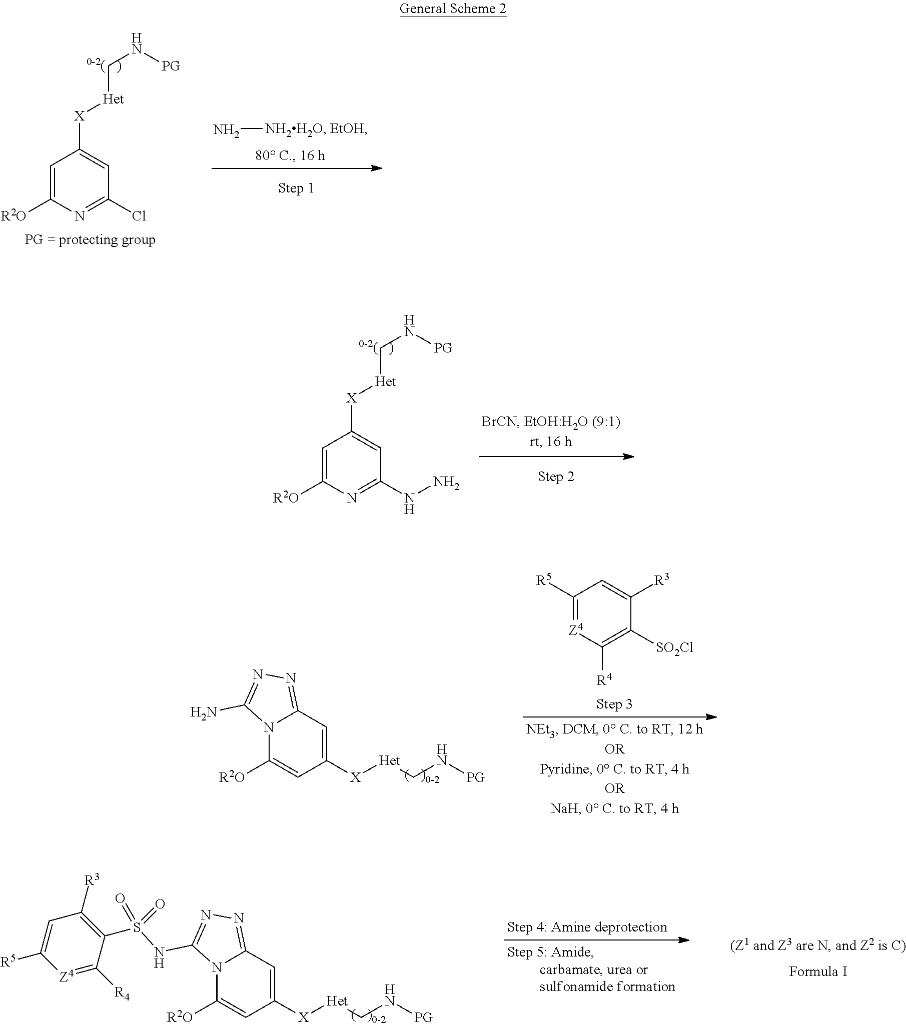

As shown, the [1,2,4]triazolo[4,3-a]pyridine compounds provided herein can be prepared by starting with an appropriately substituted 2-chloropyridine which can be converted to the corresponding 2-hydrazinopyridine by heating in presence of hydrazine hydrate (Step 1). In Step 2, reaction with cyanogen bromide leads to the assembly of the [1,2,4]triazolo[4,3-a]pyridin-3-amine scaffold. Steps 3, 4 and 5 follow protocols detailed above for General Scheme 1 (steps 2, 3 and 4) leading to the synthesis of compounds of Formula I, where Z¹ and Z³ are N and Z² is C.

General Scheme 3 describes the preparation of a compound of Formula I where Z¹ and Z² are N and Z³ is C; and all other groups are as defined in the Summary or in any embodiments described herein.

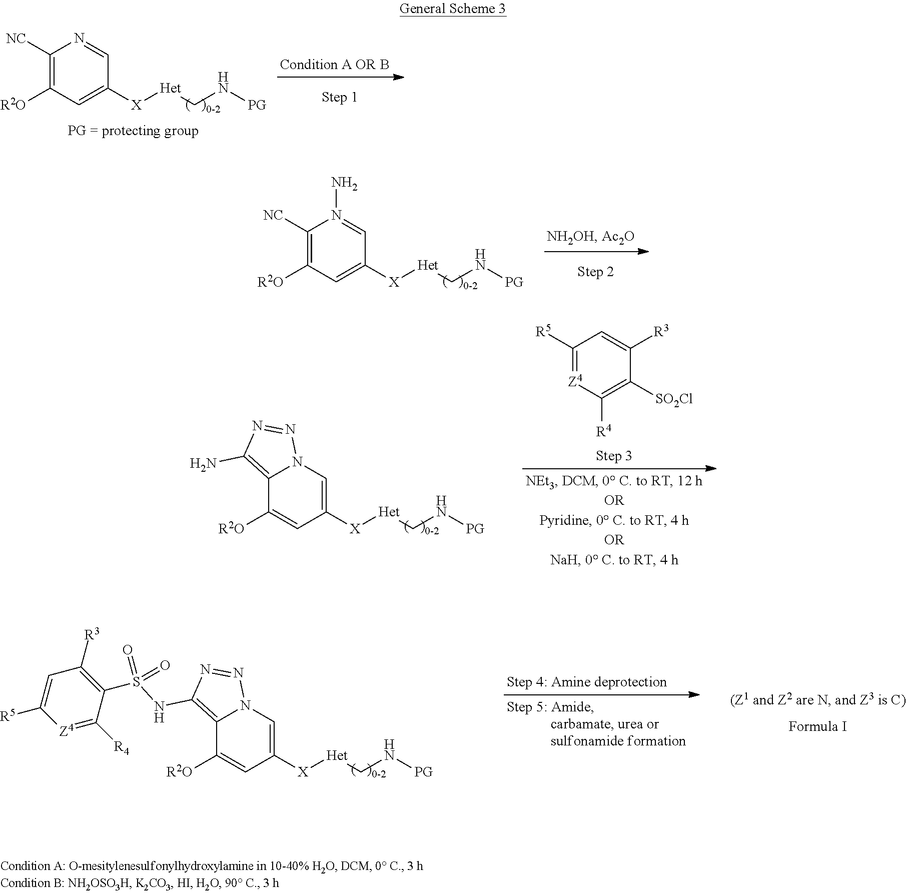

An appropriately substituted 2-cyano pyridine is converted to a 1-amino 2-cyano pyridine by reacting with a sulfonylated hydroxyl amine or (aminooxy)sulfonic acid (Step 1, condition A or B), followed by cyclization in the presence of hydroxylamine and acetic anhydride (Step 2). Steps 3, 4 and 5 follow protocols detailed above for General Scheme 1 (steps 2, 3 and 4) leading to the synthesis of compounds of Formula I, where Z¹ and Z² are N and Z³ is C.

General Scheme 4 describes the preparation of a compound of Formula I where Z¹ is NH and Z² and Z³ are C; and all other groups are as defined in the Summary or in any embodiments described herein.

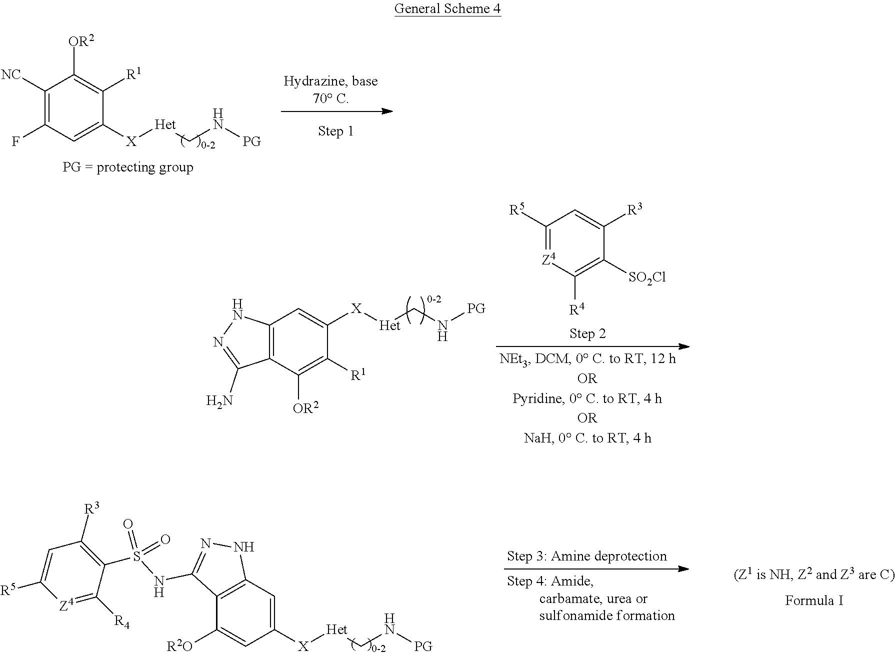

An appropriately substituted 2-fluorobenzonitrile starting material is converted to the 3-aminobenzopyrazole (Step 1), using hydrazine in the presence of a base such as potassium carbonate, as shown above. Steps 2, 3 and 4 follow protocols detailed above for General Scheme 1 (steps 2, 3 and 4) leading to the synthesis of compounds of Formula I, where Z¹ is NH and Z² and Z³ are C.

General Scheme 5 describes the preparation of a compound of Formula I where Z¹ is N, one of Z² and Z³ is C and the other of Z² and Z³ is N; and all other groups are as defined in the Summary or in any embodiments described herein.

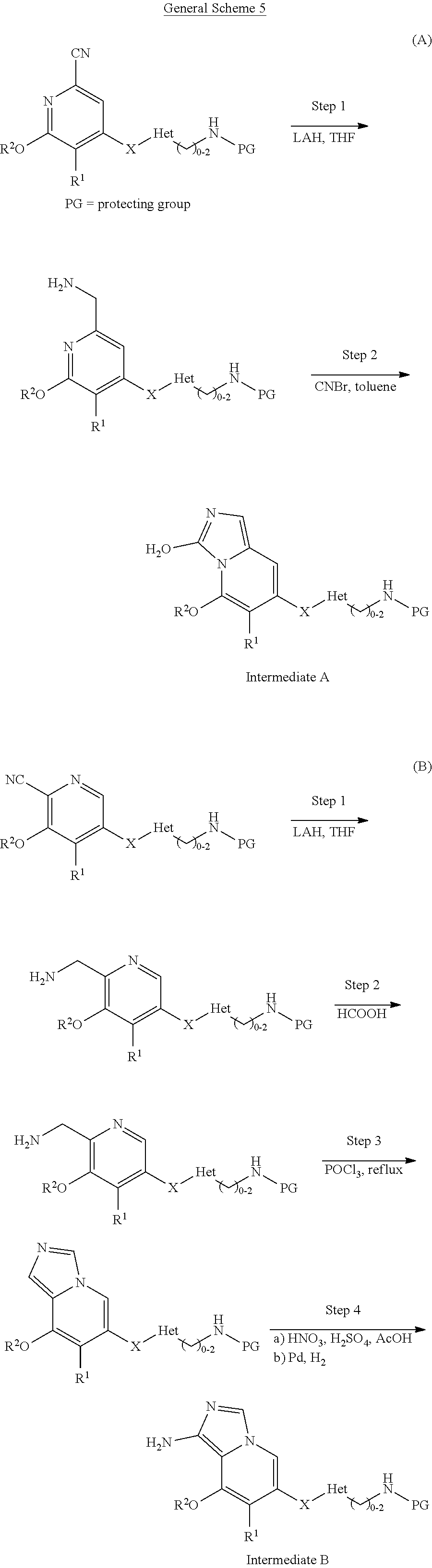

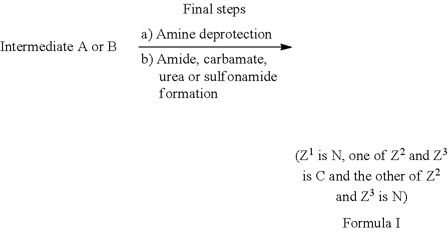

General scheme 5(A) shows that an appropriately substituted picolinonitrile is reduced in Step 1 with LAH in a solvent such as THF. Treatment of the resulting 2-aminomethyl pyridine intermediate with cyanogen bromide in toluene (Step 2) affords the imidazo[1,5-a]pyridin-3-amine Intermediate A. In General scheme 5(B), an appropriately substituted 2-cyanopyridine is reduced to the corresponding 2-aminomethyl pyridine (Step 1 as in Scheme 5A above), then subjected to Steps 2 and 3 which involve N-formylation followed by refluxing in phosphorus oxychloride to construct the imidazo[1,5-a]pyridine ring. Nitration and reduction with the reagents in Step 4 leads to the substituted imidazo[1,5-a]pyridin-1-amine, Intermediate B. In the final steps, Intermediate A and B follow protocols detailed above for General Scheme 1 (steps 2, 3 and 4) leading to the synthesis of compounds of Formula I, where Z¹ is N, one of Z² and Z³ is C and the other of Z² and Z³ is N.

General Scheme 6 describes the preparation of a compound of Formula I where R² and R⁴ together form heteroalkylene; and all other groups are as defined in the Summary or in any embodiments described herein.

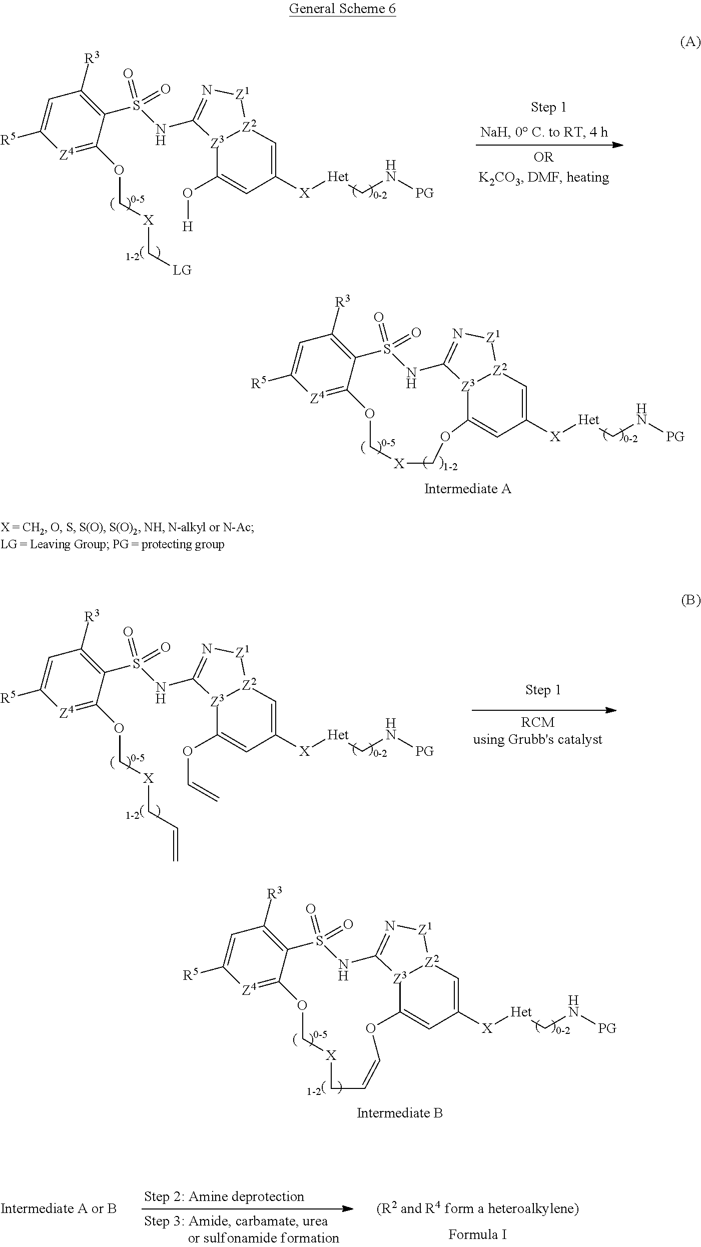

The synthesis of the macrocyclic compounds is accomplished by assembling starting materials using general methods described above. In General Scheme 6(A), R⁴ is a phenoxy or heteroaryloxy group with a terminal leaving group which reacts with a hydroxy group at R² in the presence of a base such as NaH or K₂CO₃. Alternatively, in General Scheme 6(B), R⁴ is a phenoxy or heteroaryloxy group with a terminal alkene which reacts with a terminal alkene at R² in a ring closing metathesis (RCM) reaction in the presence of Grubb's catalyst. The resulting alkene is a feature of the final molecule or is reduced to a saturated heterocycle. Steps 2 and 3 follow protocols detailed above for General Scheme 1 (steps 2, 3 and 4) leading to the synthesis of compounds of Formula I, where R² and R⁴ form a heteroalkylene.

### IV. Pharmaceutical Compositions

The pharmaceutical compositions provided herein contain therapeutically effective amounts of one or more of compounds provided herein and a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically, the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of a disease or disorder disclosed herein.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the subject treated. The therapeutically effective concentration may be determined empirically by testing the compounds in in vitro and in vivo systems described herein and then extrapolated therefrom for dosages for humans. In some embodiments, the active compound is administered in a method to achieve a therapeutically effective concentration of the drug. In some embodiments, a companion diagnostic (see, e.g., Olsen D and Jorgensen J T, Front. Oncol., 2014 May 16, 4:105, doi: 10.3389/fonC.2014.00105) is used to determine the therapeutic concentration and safety profile of the active compound in specific subjects or subject populations.

The concentration of active compound in the pharmaceutical composition will depend on absorption, tissue distribution, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of a disease or disorder disclosed herein.

In certain embodiments, a therapeutically effective dosage should produce a serum concentration of active ingredient of from about 0.1 ng/mL to about 50-100 μg/mL. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to oral, parenteral, subcutaneous, intravenous, intramuscular, intraperitoneal, intrathecal, mucosal, dermal, transdermal, buccal, rectal, topical, local, nasal or inhalation. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerin, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium croscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001%-100% active ingredient, in certain embodiments, about 0.1-85% active ingredient, or, in other embodiments, about 75-95% active ingredient.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Lactose-free compositions provided herein can contain excipients that are well known in the art and are listed, for example, in the U.S. Pharmacopeia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

### A. Oral Dosage Forms

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, crospovidone, alginic acid, corn starch, potato starch, bentonite, methylcellulose, agar and carboxymethylcellulose. Coloring agents include, for example, any of the approved certified water-soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene lauryl ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable salt thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil in-water or water in oil. In some embodiments, the suspension is a suspension of microparticles or nanoparticles. In some embodiments, the emulsion is an emulsion of microparticles or nanoparticles.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water-soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### B. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. In some embodiments, the suspension is a suspension of microparticles or nanoparticles. In some embodiments, the emulsion is an emulsion of microparticles or nanoparticles. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the subject or animal as is known in the art.

The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically, a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### C. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

### D. Topical Administration

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable salts thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### E. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams.

Tablets and capsules for rectal administration are manufactured using the same pharmaceutically acceptable substance and by the same methods as for formulations for oral administration.

### F. Sustained Release Compositions

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased subject compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

### G. Targeted Formulations

The compounds provided herein, or pharmaceutically acceptable salts thereof, may also be formulated to be targeted to a particular tissue, receptor, or other area of the body of the subject to be treated, including liposome-, resealed erythrocyte-, and antibody-based delivery systems. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, the antibody-based delivery system is an antibody-drug conjugate (“ADC”), e.g., as described in Hamilton G S, Biologicals, 2015 September, 43(5):318-32; Kim E G and Kim K M, Biomol. Ther. (Seoul), 2015 November, 23(6):493-509; and Peters C and Brown S, Biosci. Rep., 2015 Jun. 12, 35(4) pii: e00225, each of which is incorporated herein by reference.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### H. Articles of Manufacture

The compounds or pharmaceutically acceptable salts can be packaged as articles of manufacture containing packaging material, a compound or pharmaceutically acceptable salt thereof provided herein, which is used for treatment, prevention or amelioration of one or more symptoms or progression of a disease or disorder disclosed herein, and a label that indicates that the compound or pharmaceutically acceptable salt thereof is used for treatment, prevention or amelioration of one or more symptoms or progression of a disease or disorder disclosed herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In certain embodiments, provided herein also are kits which, when used by the medical practitioner, can simplify the administration of appropriate amounts of active ingredients to a subject. In certain embodiments, the kit provided herein includes a container and a dosage form of a compound provided herein, including a single enantiomer or a mixture of diastereomers thereof; or a pharmaceutically acceptable salt, solvate, or prodrug thereof.

In certain embodiments, the kit includes a container comprising a dosage form of the compound provided herein, including a single enantiomer or a mixture of diastereomers thereof; or a pharmaceutically acceptable salt, solvate, or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### V. Dosing

The compounds and pharmaceutical compositions provided herein may be dosed in certain therapeutically or prophylactically effective amounts, certain time intervals, certain dosage forms, and certain dosage administration methods as described below.

In certain embodiments, a therapeutically or prophylactically effective amount of the compound is from about 0.005 to about 1,000 mg per day, from about 0.01 to about 500 mg per day, from about 0.01 to about 250 mg per day, from about 0.01 to about 100 mg per day, from about 0.1 to about 100 mg per day, from about 0.5 to about 100 mg per day, from about 1 to about 100 mg per day, from about 0.01 to about 50 mg per day, from about 0.1 to about 50 mg per day, from about 0.5 to about 50 mg per day, from about 1 to about 50 mg per day, from about 0.02 to about 25 mg per day, from about 0.05 to about 10 mg per day, from about 0.05 to about 5 mg per day, from about 0.1 to about 5 mg per day, or from about 0.5 to about 5 mg per day.

In certain embodiments, the therapeutically or prophylactically effective amount is about 0.1, about 0.2, about 0.5, about 1, about 2, about 3, about 4, about 5, about 6, about 7, about 8, about 9, about 10, about 15, about 20, about 25, about 30, about 40, about 45, about 50, about 60, about 70, about 80, about 90, about 100, or about 150 mg per day.

In one embodiment, the recommended daily dose range of the compound provided herein, or a derivative thereof, for the conditions described herein lie within the range of from about 0.5 mg to about 50 mg per day, in one embodiment given as a single once-a-day dose, or in divided doses throughout a day. In some embodiments, the dosage ranges from about 1 mg to about 50 mg per day. In other embodiments, the dosage ranges from about 0.5 to about 5 mg per day. Specific doses per day include 0.1, 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mg per day.

In a specific embodiment, the recommended starting dosage may be 0.5, 1, 2, 3, 4, 5, 10, 15, 20, 25 or 50 mg per day. In another embodiment, the recommended starting dosage may be 0.5, 1, 2, 3, 4, or 5 mg per day. The dose may be escalated to 15, 20, 25, 30, 35, 40, 45 and 50 mg/day. In a specific embodiment, the compound can be administered in an amount of about 25 mg/day. In a particular embodiment, the compound can be administered in an amount of about 10 mg/day. In a particular embodiment, the compound can be administered in an amount of about 5 mg/day. In a particular embodiment, the compound can be administered in an amount of about 4 mg/day. In a particular embodiment, the compound can be administered in an amount of about 3 mg/day.

In certain embodiments, the therapeutically or prophylactically effective amount is from about 0.001 to about 100 mg/kg/day, from about 0.01 to about 50 mg/kg/day, from about 0.01 to about 25 mg/kg/day, from about 0.01 to about 10 mg/kg/day, from about 0.01 to about 9 mg/kg/day, 0.01 to about 8 mg/kg/day, from about 0.01 to about 7 mg/kg/day, from about 0.01 to about 6 mg/kg/day, from about 0.01 to about 5 mg/kg/day, from about 0.01 to about 4 mg/kg/day, from about 0.01 to about 3 mg/kg/day, from about 0.01 to about 2 mg/kg/day, from about 0.01 to about 1 mg/kg/day, or from about 0.01 to about 0.05 mg/kg/day.

The administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/kg/day for a 65 kg human is approximately equal to 38 mg/m²/day.

In certain embodiments, the amount of the compound administered is sufficient to provide a plasma concentration of the compound at steady state, ranging from about 0.001 to about 500 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In other embodiments, the amount of the compound administered is sufficient to provide a plasma concentration of the compound at steady state, ranging from about 5 to about 100 nM, about 5 to about 50 nM, about 10 to about 100 nM, about 10 to about 50 nM or from about 50 to about 100 nM.

As used herein, the term “plasma concentration at steady state” is the concentration reached after a period of administration of a compound provided herein, or a derivative thereof. Once steady state is reached, there are minor peaks and troughs on the time dependent curve of the plasma concentration of the compound.

In certain embodiments, the amount of the compound administered is sufficient to provide a maximum plasma concentration (peak concentration) of the compound, ranging from about 0.001 to about 50 μM, about 0.002 to about 200 μM, about 0.005 to about 100 μM, about 0.01 to about 50 μM, from about 1 to about 50 μM, about 0.02 to about 25 μM, from about 0.05 to about 20 μM, from about 0.1 to about 20 μM, from about 0.5 to about 20 μM, or from about 1 to about 20 μM.

In certain embodiments, the amount of the compound administered is sufficient to provide a minimum plasma concentration (trough concentration) of the compound, ranging from about 0.001 to about 500 μM, about 0.002 to about 200 μM, about 0.005 to about 100 μM, about 0.01 to about 50 μM, from about 1 to about 50 μM, about 0.01 to about 25 μM, from about 0.01 to about 20 μM, from about 0.02 to about 20 μM, from about 0.02 to about 20 μM, or from about 0.01 to about 20 μM.

In certain embodiments, the amount of the compound administered is sufficient to provide an area under the curve (AUC) of the compound, ranging from about 100 to about 100,000 ng*hr/mL, from about 1,000 to about 50,000 ng*hr/mL, from about 5,000 to about 25,000 ng*hr/mL, or from about 5,000 to about 10,000 ng*hr/mL.

The methods provided herein encompass treating a patient regardless of subject's age, although some diseases or disorders are more common in certain age groups.

Depending on the disease to be treated and the subject's condition, the compound provided herein, or a derivative thereof, may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. The compound provided herein, or a derivative thereof, may be formulated, alone or together, in suitable dosage unit with pharmaceutically acceptable excipients, carriers, adjuvants and vehicles, appropriate for each route of administration.

In one embodiment, the compound provided herein, or a derivative thereof, is administered orally. In another embodiment, the compound provided herein, or a derivative thereof, is administered parenterally. In yet another embodiment, the compound provided herein, or a derivative thereof, is administered intravenously.

The compound provided herein, or a derivative thereof, can be delivered as a single dose such as, e.g., a single bolus injection, or oral tablets or pills; or over time, such as, e.g., continuous infusion over time or divided bolus doses over time. The compound can be administered repeatedly if necessary, for example, until the subject experiences stable disease or regression, or until the subject experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205 216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

The compound provided herein, or a derivative thereof, can be administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug). As used herein, the term “daily” is intended to mean that a therapeutic compound, such as the compound provided herein, or a derivative thereof, is administered once or more than once each day, for example, for a period of time. The term “continuous” is intended to mean that a therapeutic compound, such as the compound provided herein or a derivative thereof, is administered daily for an uninterrupted period of at least 10 days to 52 weeks. The term “intermittent” or “intermittently” as used herein is intended to mean stopping and starting at either regular or irregular intervals. For example, intermittent administration of the compound provided herein or a derivative thereof is administration for one to six days per week, administration in cycles (e.g., daily administration for two to eight consecutive weeks, then a rest period with no administration for up to one week), or administration on alternate days. The term “cycling” as used herein is intended to mean that a therapeutic compound, such as the compound provided herein or a derivative thereof, is administered daily or continuously but with a rest period. In some such embodiments, administration is once a day for two to six days, then a rest period with no administration for five to seven days.

In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, the compound provided herein, or a derivative thereof, is administered once a day. In another embodiment, the compound provided herein, or a derivative thereof, is administered twice a day. In yet another embodiment, the compound provided herein, or a derivative thereof, is administered three times a day. In still another embodiment, the compound provided herein, or a derivative thereof, is administered four times a day.

In certain embodiments, the compound provided herein, or a derivative thereof, is administered once per day from one day to six months, from one week to three months, from one week to four weeks, from one week to three weeks, or from one week to two weeks. In certain embodiments, the compound provided herein, or a derivative thereof, is administered once per day for one week, two weeks, three weeks, or four weeks. In one embodiment, the compound provided herein, or a derivative thereof, is administered once per day for 4 days. In one embodiment, the compound provided herein, or a derivative thereof, is administered once per day for 5 days. In one embodiment, the compound provided herein, or a derivative thereof, is administered once per day for 6 days. In one embodiment, the compound provided herein, or a derivative thereof, is administered once per day for one week. In another embodiment, the compound provided herein, or a derivative thereof, is administered once per day for two weeks. In yet another embodiment, the compound provided herein, or a derivative thereof, is administered once per day for three weeks. In still another embodiment, the compound provided herein, or a derivative thereof, is administered once per day for four weeks.

### VI. Methods of Treatment

Inhibitors of post-translational lysine acetylation mediated by KATs of the MYST family are promising anti-neoplastic agents and therefore are useful therapeutic agents, e.g., for use in the treatment of cancer. Such agents are also useful as therapeutic agents for the treatment of cancers which exhibit overexpression of MYST proteins.

In another embodiment, provided is a method of treating a subject with cancer by administering a compound or composition provided herein. In another embodiment, provided herein is a method of treating a condition, disease, or disorder by inhibiting MYST family of lysine acetyl transferases, including KAT6A and KAT6B, comprising administering a therapeutically effective amount of a compound or composition provided herein.

In another embodiment, provided is a method of treating a condition, disease, or disorder, where the condition, disease, or disorder is a hyperproliferative disease, such as cancer. In some embodiments, the cancer is selected from: lymphoma, melanoma, carcinoma (e.g. adenocarcinoma, hepatocellular carcinoma, medullary carcinoma, papillary carcinoma, squamous cell carcinoma), astrocytoma, glioma, medulloblastoma, myeloma, meningioma, neuroblastoma, and sarcoma (e.g. angiosarcoma, chrondrosarcoma, osteosarcoma). In some embodiments, the cancer is a MYST overexpressing cancer. In another embodiment, the cancer over-expresses MYST protein relative to non-cancerous tissue. In some embodiments, the cancer overproduces MYST mRNA relative to non-cancerous tissue. In another embodiment, the cancer is a MYST overexpressing cancer where the overexpressed MYST protein or MYST mRNA is any one of KATs of the MYST family, e.g., KAT6A. In some embodiments, the cancer is leukemia, acute lymphocytic leukemia (ALL), acute myeloid leukemia (AML), chronic lymphocytic leukemia (CLL), chronic myeloid leukemia (CML), non-Hodgkin's lymphoma, Hodgkin's disease, prostate cancer, lung cancer, melanoma, breast cancer, breast ductal carcinoma, colon and rectal cancer, colon cancer, squamous cell carcinoma, gastric cancer, adrenocortical cancer, anal cancer, bladder cancer, blood cancer, bone cancer, brain tumor, cancer of the female genital system, cancer of the male genital system (including testicular cancer and penile cancer), central nervous system lymphoma, cervical cancer, childhood rhabdomyosarcoma, childhood sarcoma, endometrial cancer, endometrial sarcoma, esophageal cancer, eye cancer, gallbladder cancer, gastrointestinal tract cancer, hairy cell leukemia, head and neck cancer, hepatocellular cancer, hypopharyngeal cancer, Kaposi's sarcoma, kidney cancer, laryngeal cancer, liver cancer, malignant fibrous histiocytoma, malignant thymoma, mesothelioma, multiple myeloma, myeloma, nasal cavity and paranasal sinus cancer, nasopharyngeal cancer, nervous system cancer, neuroblastoma, oral cavity cancer, oropharyngeal cancer, osteosarcoma, ovarian cancer, pancreatic cancer, parathyroid cancer, pharyngeal cancer, pituitary tumor, plasma cell neoplasm, primary CNS lymphoma, rectal cancer, respiratory system, retinoblastoma, salivary gland cancer, skin cancer, small intestine cancer, soft tissue sarcoma, stomach cancer, testicular cancer, thyroid cancer, urinary system cancer, uterine cancer, uterine sarcoma, vaginal cancer, endocrine, neoplasms of the central nervous system (CNS), primary CNS lymphoma, spinal axis tumors, glioblastoma, brain stem glioma, pituitary adenoma, vascular system, Waldenstrom's macroglobulinemia or Wilms' tumor. In some embodiments, the cancer is breast cancer, including ER positive breast cancer, non-small cell lung cancer, prostate cancer, pancreatic cancer, ovarian cancer, or blood cancer (including a leukemia or lymphoma). In one embodiment, the cancer is breast cancer, lung cancer, ovarian cancer, colon cancer, prostate cancer, uterine/cervical cancer or leukemia.

In another embodiment, provided is a method of inhibiting the activity of a MYST KAT by contacting the MYST KAT or a composition comprising the MYST KAT with a compound or composition provided herein. In one embodiment, the MYST KAT is a KAT6 lysine acetyl transferase. In another embodiment, the MYST KAT is KAT6A.

Without wishing to be bound by any theory, one aspect of the catalytic mechanism of the KAT family of enzymes is the acetylation of lysine residues using the cofactor Ac-CoA as an acetyl donor. One possible catalytic mechanism is the stepwise transfer of the acetyl group from acetyl co-enzyme A (Ac-CoA) to transiently form an acetyl-enzyme intermediate. This acetyl-enzyme intermediate subsequently is the source of the acetyl group that is transferred to the substrate, typically a lysine on a histone (Wapenaar et al., 2016, supra). In the MYST family of KATs there is a conserved cysteine that is acylated transiently as part of the catalytic cycle, as indicated by the bold C in the sequences below.

Amino Acid Sequence Alignment near the Ac-CoA binding site of the MYST Family of KATs

KAT5
(SEQ ID NO: 1)
ESTEDYNVACILTLPPYQRR
KAT8
(SEQ ID NO: 2)
ESPDGNNVACILTLPPYQRR
KAT7
(SEQ ID NO: 3)
NSFLNYNVSCILTMPQYMRQ
KAT6A
(SEQ ID NO: 4)
HCQQKYNVSCIMILPQYQRK
KAT6B
(SEQ ID NO: 5)
LCQQKYNVSCIMIMPQHQRQ

Thus, in one embodiment, provided is a method of inhibiting a MYST KAT by covalent modification of this conserved cysteine residue with a compound or composition provided herein. In another embodiment, provided is a method of covalent modification of this conserved MYST KAT cysteine residue by contacting the MYST KAT or a composition containing the MYST KAT with a compound or composition provided herein.

In another embodiment, it is known in the art that histone acetyltransferases or HATs, including KAT6, have a key role in facilitating myc-driven transcriptional regulation. Myc is a transcriptional regulator that is known to recruit HATs to target chromatin facilitating chromatin remodeling, resulting in transcription, proliferation, and tumor growth. Myc is a transcriptional regulator with many protein interactors in a series of complexes, which mediate this transcriptional activity. One such interacting protein known as transformation/transcription domain associated protein or TRRAP, is a member of several HAT containing protein complexes where it serves as bridging protein between transcription factors like myc and HATs. Myc has been documented to recruit histone acetylation through a TRRAP complex facilitating acetylation of nucleosomes at histone H4 through HATs that include but may not be limited to KAT2A and KAT5 and Tip60/p400. This myc-associated HAT activity enables the opening of chromatin structurally facilitating transcription (see, e.g., Laurenco, et al. Nature Reviews Cancer 2021, 21, 579-591). Myc recruits HATs to target chromatin and locally promote acetylation of multiple specific lysines on both H3 and H4 some of which are known targets of KAT6A acetylation, resulting in transcriptional activation (see, e.g., Martinato et al. PLoS ONE 2008, 3(11), e3650, doi:10.1371/journal.pone.0003650). KAT6A, as a recruitable HAT, plays a role in specific cancers and cell types and may be a key mediator of HAT activity recruited to myc-driven transcriptional activation sites in some tumors.

Furthermore, hallmark targets of myc-induced transcriptional regulation (myc pathway targets) are reduced through the use of KAT6A inhibitors. This includes the myc pathway in KAT6A-amplified breast cancer cell lines following administration of a KAT6 inhibitor (See, e.g., Sharma et al. Cell Chemical Biology 2023, doi.org/10.1016/j.chembiol.2023.07.005).

Indeed, there is direct evidence that KAT6 may be a HAT that has a key role in myc-driven transcriptional regulation and that loss of KAT6 activity could disrupt myc-driven transcriptional programming. Myc-driven growth of a lymphoma cell line in vivo is inhibited by systemic administration of KAT6 inhibitors. Specifically, two small molecule inhibitors of KAT6A and KAT6B lead to anti-tumor activity in MYC-driven models of lymphoma (see, e.g., Baell, et al. Nature 2018, 560, 253-257; Priebbenow et al. J. Med. Chem. 2020, 63, 4655-4684)

Finally, MYC is amplified in approximately 30% of solid tumors, including lung, ovarian, esophagus, breast, stomach, pancreas and liver cancer. There are a number of overlapping solid tumor types with characterized myc activity which also contain KAT6 genomic amplifications. Therefore, inhibition of KAT6A activity is expected to reduce transcriptional activity and tumorigenicity in solid tumors in which myc is a key transcriptional regulator.

Thus, in another embodiment, provided is a method of treating a myc-driven cancer in a subject by administering a compound or composition provided herein to the subject. In another embodiment, the myc-driven cancer is lung, ovarian, esophageal, breast, stomach, pancreatic or liver cancer. In another embodiment, the myc-driven cancer is a cancer in which myc is amplified. In another embodiment, provided is a method of treating a cancer in which myc is amplified in a subject by administering a compound or composition provided herein to the subject.

KAT6A is an acetyltransferase of p53 at K120 and K382 and colocalizes with p53 in promyelocytic leukemia (PML) nuclear bodies following cellular stress (Rokudai, et al. PNAS 2013, 110(10), 3895-3900). This ternary complex enhances p53-dependent p21 expression. KAT6A acetylation of p53, affecting complex formation or stability and altering its downstream activity, is thus involved in pathogenesis of tumors and leukemia. Therefore, KAT6A inhibitors can be used in the treatment of hematological cancers. Thus, in another embodiment, provided is a method of treating a hematological cancer in a subject by administering a compound or composition provided herein to the subject.

KAT6A acetylation of SMAD3 regulates myeloid-derived suppressor cell recruitment, metastasis, and immunotherapy in triple-negative breast cancer (TNBC) (Yu, et al. Adv. Sci. 2021, 8, 2100014). Therefore, KAT6A inhibitors can be used in the treatment of TNBC. In another embodiment, provided is a method of treating TNBC in a subject by administering a compound or composition provided herein to the subject.

### VII. Combination Therapy with Other Active Agent(s)

The compound provided herein, or a derivative thereof, can also be combined or used in combination with other therapeutic agents useful in the treatment and/or prevention of hyperproliferative diseases including cancer.

In one embodiment, provided herein is a method of treating, preventing, or managing hyperproliferative diseases including cancer, comprising administering to a subject a compound provided herein, or a derivative thereof; in combination with one or more second active agents.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein, or a derivative thereof) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

Administration of the compound provided herein, or a derivative thereof and one or more other active agents to a subject can occur simultaneously or sequentially by the same or different routes of administration. The suitability of a particular route of administration employed for a particular active agent will depend on the active agent itself (e.g., whether it can be administered orally without decomposing prior to entering the blood stream) and the disease or disorder being treated.

The route of administration of the compound provided herein, or a derivative thereof, is independent of the route of administration of a second therapy. In one embodiment, the compound provided herein, or a derivative thereof, is administered orally. In another embodiment, the compound provided herein, or a derivative thereof, is administered intravenously. Thus, in accordance with these embodiments, the compound provided herein, or a derivative thereof, is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, the compound provided herein, or a derivative thereof, and a second therapy are administered by the same mode of administration, orally or by IV. In another embodiment, the compound provided herein, or a derivative thereof, is administered by one mode of administration, e.g., by IV, whereas the second agent is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of the compound provided herein, or a derivative thereof, and any optional additional active agents concurrently administered to the subject.

One or more other active ingredients or agents can be used together with the compound provided herein, or a derivative thereof, in the methods and compositions provided herein. Other active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies, immune-based therapeutic agents in classification known as immunoncology which improve immune-mediated recognition of tumors, particularly, therapeutic antibodies to cancer antigens. Typical large molecule active agents are biological molecules, such as naturally occurring or synthetic or recombinant proteins.

In one embodiment, the compound provided herein, or a derivative thereof, are administered in an amount ranging from about 0.1 to about 150 mg, from about 1 to about 25 mg, or from about 2 to about 10 mg orally and daily alone, or in combination with a second active agent, prior to, during, or after the use of conventional therapy.

In another embodiment, the choice of other active agent depends on the disease to be treated. Examples of second active agents for a particular cancer are well known to those of skill in the art. For example, if the disease is breast cancer, then the second active agent may be an aromatase inhibitor, a CDK4/6 inhibitor or an ER antagonist. Other examples of agents that can be used in combination with a KAT6 inhibitor for treatment of breast cancer are selective inhibitors of Cyclin Dependent Kinases such as CDK2 or CDK4 inhibitors and Selective Estrogen Receptor Degraders (SERDs). PIK3CA is a frequently mutated oncogene in breast cancer. The H1047R mutation of PIK3CA is highly prevalent in breast cancers and other solid tumors. KAT6 inhibitors can be combined with mutant-selective PI3Kα inhibitors for the treatment of breast and other cancers.

Another example of a solid tumor type which harbors KAT6A genomic and mRNA amplifications is ovarian cancer. Ovarian cancers are treated with platinum-based chemotherapeutics such as cisplatin, which have a high level of toxicity that often limits dosing and achieving maximal efficacious exposures. In some cases, PARP inhibitors or CDK2 inhibitors are used to treat ovarian cancers. KAT6 inhibitors can be used in combination with these agents, but not limited to these agents, to treat ovarian cancers.

KAT6 epigenetic activity has been shown to be involved in the development of resistance to menin pathway inhibitors and has been demonstrated to play a role in chromatin remodeling by the menin-MLL/KMT2 complex in solid tumors as well as AML. KAT6 inhibitors can be applied in the treatment of hematological cancers such as AML and in tumors where the menin-MLL/KMT2 complex drives tumors growth, alone and in combination with menin and menin pathway inhibitors.

### VIII. EXAMPLES

The examples below are meant to illustrate certain embodiments provided herein, and not to limit the scope of this disclosure.

As used herein, the symbols and conventions used in these processes, schemes, and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); h, hr or hrs (hours); min (minutes); MS (mass spectrometry); ESI (electrospray ionization); rt (room temperature); Rf (retention factor); TLC (thin-layer chromatography); LCMS (liquid chromatography-mass spectrometry); HPLC (high performance liquid chromatography); AcOH (acetic acid); n-BuLi (n-Butyl lithium); tBuOK (potassium tert butoxide); CDCl₃ (Chloroform-d); CH₃CN (acetonitrile); Cs₂CO₃ (cesium carbonate); DMF (N,N-Dimethylformamide); DCM (dichloromethane); DEA (diethylamine); DIPEA (diisopropyl ethylamine); DMSO (dimethylsulfoxide); DMSO-d₆ (dimethyl sulfoxide-d₆); EtOAc or EA (ethyl acetate); Et₃N (triethylamine); EtOH (ethanol); HCl (hydrochloric acid); H₂SO₄ (sulphuric acid); K₂CO₃ (potassium carbonate); LiOH (lithium hydroxide); MsCl (methane sulphonyl chloride); Mel (methyl iodide); MeOH (Methanol); MeOH-d₄ (Methanol-d₄); NaBH₄ (sodium borohydride); NaH (sodium hydride); NaHCO₃ (sodium bicarbonate); NaNO₂ (sodium nitrite); NaOH (sodium hydroxide); NaOMe (sodium methoxide); Na₂SO₄ (sodium sulphate); K₃FeCN₆ (potassium ferricyanide); Pd(PPh₃)₄ (tetrakis(triphenylphosphine)palladium(O)); SO₂ (sulphur dioxide); SO₂Cl₂ (sulfuryl chloride); T₃P (propanephosphonic acid anhydride); THF (tetrahydrofuran); TMEDA (1,2 bis(dimethylamino)ethane); Zn (zinc); ZnCN₂ (zinc cyanide).

For all the following examples, standard work-up and purification methods known to those skilled in the art can be utilized. Unless otherwise indicated, all temperatures are expressed in ° C. (degrees Celsius). All reactions are conducted at room temperature unless otherwise noted. Synthetic methodologies illustrated herein are intended to exemplify the applicable chemistry through the use of specific examples and are not indicative of the scope of the disclosure.

### Examples 1-2

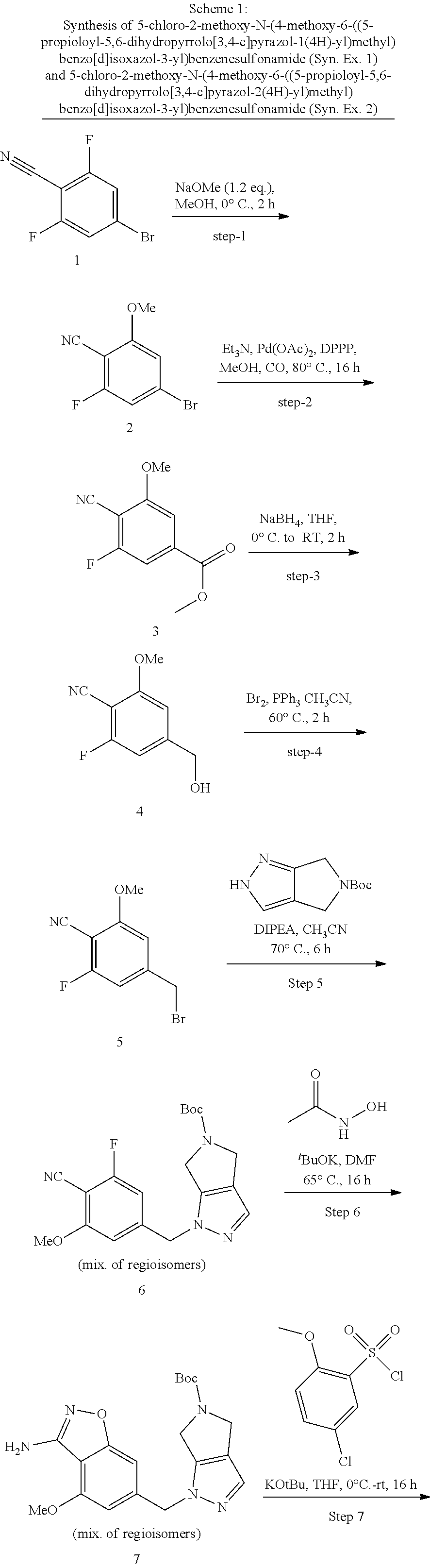

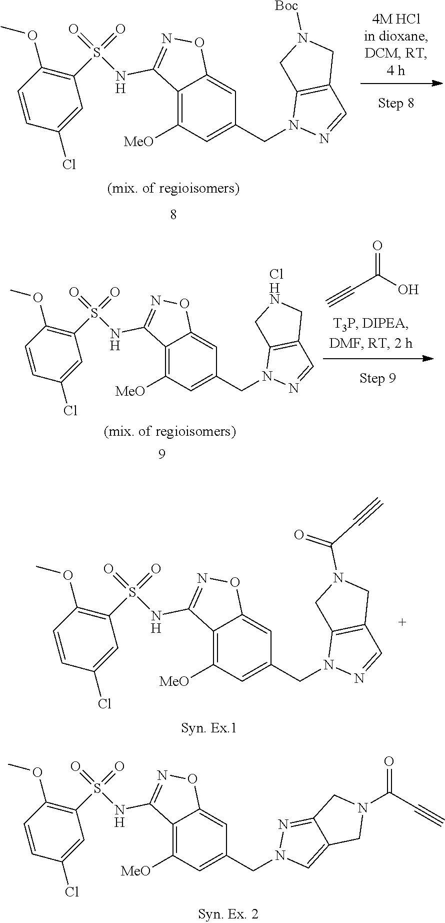

### Synthesis of 4-bromo-2-fluoro-6-methoxybenzonitrile (2)

To a stirred solution of compound 1 (50 g, 229.35 mmol) in MeOH (500 mL) was added NaOMe (14.86 g, 275.22 mmol) and the reaction mixture was allowed to stir at 0° C. for 2 h. After completion (monitored by TLC), the mixture was quenched with ice cold water and extracted with ethyl acetate. The organic layer was dried over anhydrous Na₂SO₄ and concentrated under reduced pressure to afford the title compound 2 (55 g) as a yellow solid. LCMS Calculated for C8H5BrFNO: 228.95; Found: 229.5 (M+1).

### Synthesis of methyl 4-cyano-3-fluoro-5-methoxybenzoate (3)

To a solution of compound 2 (13 g, 56.511 mmol) in methanol (140 mL) in steel bomb wad added triethylamine (1.58 mL, 11.303 mmol) and purged with nitrogen for 5 min. Palladium(II) acetate (634 mg, 2.823 mmol) and DPPP (1,3-Bis(diphenylphosphino)propane) (2.33 g, 5.59 mmol) were added and reaction mixture was heated at 80° C. in presence of CO gas (110 psi) for 16 h. After completion (monitored by TLC), the mixture was cooled to room temperature and filtered through a celite bed. The filtrate was concentrated in vacuo and purified by combi flash chromatography using 14% EtOAc/Hexane to afford methyl 4-cyano-3-fluoro-5-methoxy-benzoate 3 (10 g, 47.81%) as a white solid. TLC: 20% EtOAc/Heptane (R_{f}: 0.5). LCMS Calculated form C10H8FNO3: 209.05; Found: 210.0 (M+1).

### Synthesis of 2-fluoro-4-(hydroxymethyl)-6-methoxybenzonitrile (4)

To a stirred solution of LiBH₄ (2.08 g, 95.611 mmol in THF (100 mL) at 0° C. was added compound 3 (10 g, 47.80 mmol) in THF (600 mL) dropwise. The reaction mixture was stirred at 80° C. for 4 h. After completion (monitored by TLC), the mixture was cooled to RT and quenched with ice cold water and extracted with ethyl acetate. The organic layer was washed with brine, dried over anhydrous Na₂SO₄ and concentrated under reduced pressure to afford the title compound 4 (6 g, 69.27%) as a white solid. TLC: 50% EtOAc/Heptane (R_{f}: 0.25). LCMS Calculated for C₉H8FNO2: 181.05; Found: 182.0 (M+1).

### Synthesis of 4-(bromomethyl)-2-fluoro-6-methoxybenzonitrile (5)

To a stirred solution of compound 4 (7 g, 38.637 mmol) in ACN (70 mL) was added and PPh₃ (16.82 g, 64.138 mmol) followed by Br₂ (3.08 mL, 64.137 mmol) at 0° C. The reaction mixture was stirred at 60° C. for 2 h. After completion (monitored by TLC), the mixture was quenched with sodium thiosulphate (110 mL) and extracted with EtOAc (3×60 mL). The combined organic layer was dried over anhydrous Na₂SO₄ and concentrated in vacuo. The crude was purified by combi-flash chromatography (15% EtOAc in Heptane) to afford the title compound 5 (4 g, 16.389 mmol, 42.46%) as a white solid. TLC: 30% EtOAc/Heptane (R_{f}: 0.6). LCMS Calculated for C₉H7BrFNO: 242.97; Found: 244.2 (M+2).

### Synthesis of tert-butyl 2-(4-cyano-3-fluoro-5-methoxybenzyl)-2,6-dihydropyrrolo[3,4-c]pyrazole-5(4H)-carboxylate and tert-butyl 2-(4-cyano-3-fluoro-5-methoxybenzyl)-2,6-dihydropyrrolo[3,4-c]pyrazole-5(4H)-carboxylate (6)

To a stirred solution of compound 5 (1.0 g, 4.097 mmol) in acetonitrile (5 mL) was added DIPEA (2.146 mL, 12.292 mmol) followed by tert-butyl 2,6-dihydropyrrolo[3,4-c]pyrazole-5(4H)-carboxylate (1.02 g, 4.916 mmol). The reaction mixture was heated at 70° C. for 6 h. After completion (monitored by TLC), the reaction mixture was cooled to room temperature, quenched with water and extracted with ethyl acetate. The organic layer was washed with brine, dried over anhydrous Na₂SO₄ and concentrated under reduced pressure. The crude was purified by silica gel (100-200 mesh) column chromatography using a gradient method of 0-50% EtOAc/Heptane to afford the title compound 6 (1.0 g, 48.65%, isolated as inseparable mixture regioisomers) as a yellow solid. TLC: 80% EtOAc/Heptane (R_{f}: 0.45). LCMS Calculated for C₁₉H₂₁FN₄O₃: 372.16; Found: 373.40 (M+1).

### Synthesis of tert-butyl 1-((3-amino-4-methoxybenzo[d]isoxazol-6-yl)methyl)-4,6-dihydropyrrolo[3,4-c]pyrazole-5(1H)-carboxylate and tert-butyl 2-((3-amino-4-methoxybenzo[d]isoxazol-6-yl)methyl)-2,6-dihydropyrrolo[3,4-c]pyrazole-5(4H)-carboxylate (7)

To a stirred solution of compound 6 (mixture of regioisomers) (0.50 g, 1.34 mmol) in DMF (7 mL) was added acetohydroxamic acid (0.31 g, 4.02 mmol) followed by K₂CO₃ (0.56 g, 4.02 mmol). The reaction mixture was allowed to stir at 65° C. for 16 h. After completion (monitored by TLC), the mixture was cooled to room temperature and quenched with ice water. The aqueous layer was extracted with ethyl acetate. The organic layer was washed with brine, dried over anhydrous Na₂SO₄ and concentrated under reduced pressure. The crude was purified by silica gel (100-200 mesh) column chromatography using a gradient method of 0-50% EtOAc/Heptane to afford the title compound 7 (mixture of isomers) (0.20 g, 38.64%, isolated as inseparable mixture of regioisomers) as an off-white solid. TLC: 70% EtOAc/Heptane (R_{f}: 0.40). LCMS Calculated for Cl₉H₂₃N₅O₄: 385.42; Found: 386.00 (M+1). ¹H NMR is complicated and indicates a regioisomeric mixture.

### Synthesis of tert-butyl 1-((3-((5-chloro-2-methoxyphenyl) sulfonamido)-4-methoxybenzo[d]isoxazol-6-yl) methyl)-4,6-dihydropyrrolo[3,4-c]pyrazole-5(1H)-carboxylate and tert-butyl 2-((3-((5-chloro-2-methoxyphenyl)sulfonamido)-4-methoxybenzo[d]isoxazol-6-yl)methyl)-2,6-dihydropyrrolo[3,4-c]pyrazole-5(4H)-carboxylate (8)

To a stirred solution of compound 7 (0.75 g, 1.945 mmol) in THF (20 mL) was added 1M solution of KOtBu in THF (5.8 mL, 5.838 mmol) at 0° C. The reaction mixture was stirred for 10 min. at 0° C. followed by the addition of 5-chloro-2-methoxybenzenesulfonyl chloride (0.70 g, 2.919 mmol). The reaction mixture was stirred at room temperature for 16 h. After completion (monitored by TLC), the mixture was quenched with water and extracted with EtOAc. The organic layer was dried over anhydrous Na₂SO₄, filtered, and concentrated under reduced pressure. The crude was purified by combi flash column chromatography by using 50-60% EA in Heptane to afford title compound 8 (mixture of regioisomers) (0.50 g, 43.55%) as an off-white solid. TLC: 50% EtOAc/Heptane (R_{f}: 0.5). LCMS calculated for C26H28ClN5O7S: 589.14; found: 590.20 (M+1).

### Synthesis of 5-chloro-N-(6-((5,6-dihydropyrrolo[3,4-c]pyrazol-1(4H)-yl) methyl)-4-methoxybenzo[d]isoxazol-3-yl)-2-methoxybenzenesulfonamide hydrochloride and 5-chloro-N-(6-((5,6-dihydropyrrolo[3,4-c]pyrazol-2(4H)-yl)methyl)-4-methoxybenzo[d]isoxazol-3-yl)-2-methoxybenzenesulfonamide hydrochloride (9)

To a stirred solution of compound 8 (0.50 g, 0.8475 mmol) in DCM (10 mL) was added 4M HCl in 1,4-dioxane (5 mL) at 0° C. The reaction mixture was stirred at room temperature for 4 h. After completion (monitored by TLC), the mixture was concentrated under reduced pressure. The residue was washed with Et₂O and dried under reduced pressure to afford title compound 9 (mixture of regioisomers) (0.42 g, 94.17%) as an off-white solid. TLC: 10% MeOH/DCM (R_{f}: 0.3). LCMS calculated for C21H20ClN5O5S: 489.09; found: 490.10 (M+1).

### Synthesis of 5-chloro-2-methoxy-N-(4-methoxy-6-((5-propioloyl-5,6-dihydropyrrolo[3,4-c]pyrazol-1(4H)-yl)methyl)benzo[d]isoxazol-3-yl)benzenesulfonamide (Syn. Ex. 1) and 5-chloro-2-methoxy-N-(4-methoxy-6-((5-propioloyl-5,6-dihydropyrrolo[3,4-c]pyrazol-2(4H)-yl)methyl)benzo[d]isoxazol-3-yl)benzenesulfonamide (Syn. Ex. 2)

To a stirred solution of compound 9 (0.20 g, 0.379 mmol) in DMF (5 mL) was added propiolic acid (0.032 g, 0.455 mmol), DIPEA (0.198 mL, 1.137 mmol) and 50% solution of T₃P in EtOAc (0.36 g, 1.139 mmol) at 0° C. under N₂ atmosphere. The reaction mixture was stirred at room temperature for 2 h. After completion (monitored by TLC), the reaction mixture was quenched with water and extracted with EtOAc. The organic layer was dried over anhydrous Na₂SO₄, filtered, and concentrated under reduced pressure. The crude was purified by combi flash column chromatography by using 5% MeOH in DCM to afford a mixture of regioisomers which was further purified by chiral HPLC to get 5-chloro-2-methoxy-N-(4-methoxy-6-((5-propioloyl-5,6-dihydropyrrolo[3,4-c]pyrazol-1(4H)-yl)methyl)benzo[d]isoxazol-3-yl)benzenesulfonamide (Syn. Ex. 1; 35 mg, 16.15%) as an off white solid and 5-chloro-2-methoxy-N-(4-methoxy-6-((5-propioloyl-5,6-dihydropyrrolo[3,4-c]pyrazol-2(4H)-yl)methyl)benzo[d]isoxazol-3-yl)benzenesulfonamide (Syn. Ex. 2; 15 mg, 7.28%) as an off white solid. TLC: 10% MeOH/DCM (R_{f}: 0.3), (See Table 1 for analytical data).

### Example 3

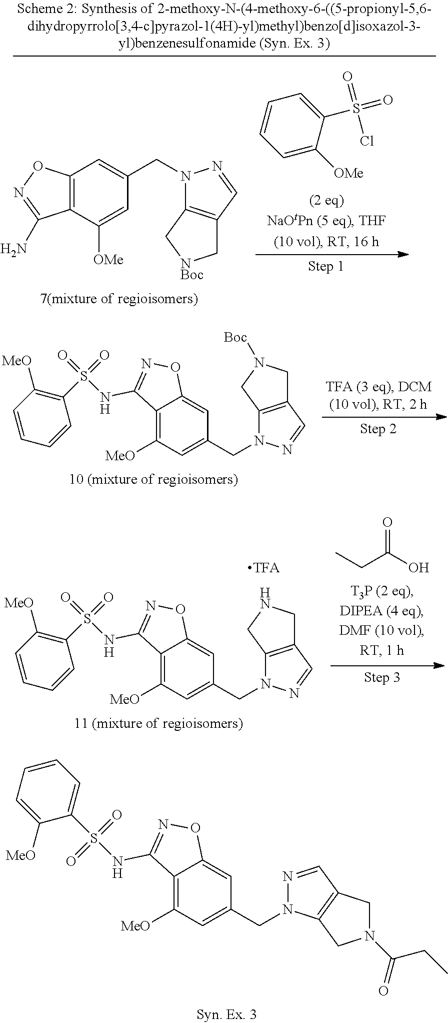

### Synthesis of tert-butyl 1-((4-methoxy-3-((2-methoxyphenyl) sulfonamido) benzo[d]isoxazol-6-yl) methyl)-4,6-dihydropyrrolo[3,4-c]pyrazole-5(1H)-carboxylate and tert-butyl 2-((4-methoxy-3-((2-methoxyphenyl)sulfonamido)benzo[d]isoxazol-6-yl)methyl)-2,6-dihydropyrrolo[3,4-c]pyrazole-5(4H)-carboxylate (10)

To a stirred solution of compound 7 (0.3 g, 0.77 mmol) in THF (5 mL) was added 2-methoxybenzenesulfonyl chloride (0.320 g, 1.55 mmol) and NaO^{t}Pn (0.428 g, 3.89 mmol). The reaction mixture was allowed to stir at room temperature for 16 h. After completion (monitored by TLC), the mixture was quenched with ice-cold water and extracted with ethyl acetate. The organic layer was washed with brine, dried over anhydrous Na₂SO₄, and concentrated under reduced pressure. The crude compound was purified by combi flash using gradient of 70-80% ethyl acetate to afford the title compound 10 (mixture of regioisomers) (310 mg, 71.75%) as brown solid. TLC: 5% MeOH/DCM (R_{f}: 0.6). LCMS Calculated for C₂₆H₂₉N₅O₇S: 555.18; Found: 556.20 (M+1).

### Synthesis of 2-methoxy-N-(4-methoxy-6-((5-(2,2,2-trifluoroacetyl)-5,6-dihydro-514-pyrrolo[3,4-c]pyrazol-1(4H)-yl)methyl)benzo[d]isoxazol-3-yl)benzenesulfonamide and 2-methoxy-N-(4-methoxy-6-((5-(2,2,2-trifluoroacetyl)-5,6-dihydro-514-pyrrolo[3,4-c]pyrazol-2(4H)-yl)methyl)benzo[d]isoxazol-3-yl)benzenesulfonamide (11)

To a stirred solution of compound 10 (mixture of regioisomers) (0.4 g, 0.68 mmol) in DCM (4 mL) was added TFA (0.52 mL, 6.83 mmol) at 0° C. The reaction was allowed to stir at the room temperature for 2 h. After completion (monitored by TLC), the reaction mixture was concentrated under reduced pressure, triturated with ether, and dried on vacuum to afford the title compound 11 (mixture of regioisomers) (410 mg, crude) as a brown solid. TLC: 10% MeOH/DCM (R_{f}:0.5). LCMS Calculated for C21H21N5O5S: 455.13: Found: 454.3 (M−1).

### Synthesis of 2-methoxy-N-(4-methoxy-6-((5-propionyl-5,6-dihydropyrrolo[3,4-c]pyrazol-1(4H)-yl)methyl)benzo[d]isoxazol-3-yl)benzenesulfonamide (Syn. Ex. 3)

To a stirred solution of compound 11 (260 mg, 0.570 mmol) in DMF (3 mL) was added propionic acid (63 mg, 0.856 mmol), DIPEA (0.398 mL, 2.28 mmol) and 50% solution of T₃P in EtOAc (0.867 mL, 1.141 mmol) at 0° C. The reaction mixture was stirred at room temperature for 2 h. After completion (monitored by TLC), the mixture was quenched with ice-cold water and extracted with EtOAc (2×50 mL). The organic layer was washed with brine, dried over anhydrous Na₂SO₄ and concentrated under reduced pressure. The crude was triturated with Et₂O and purified by chiral HPLC purification to get 2-methoxy-N-(4-methoxy-6-((5-propionyl-5,6-dihydropyrrolo[3,4-c]pyrazol-1(4H)-yl)methyl)benzo[d]isoxazol-3-yl)benzenesulfonamide (Syn. Ex. 3; 50 mg, 17.18%) as an off white solid. The other regioisomer was not isolated in pure form from the mixture TLC: 5% MeOH/DCM (R_{f}: 0.4). (See Table 1 for analytical data)

### Examples 4-5

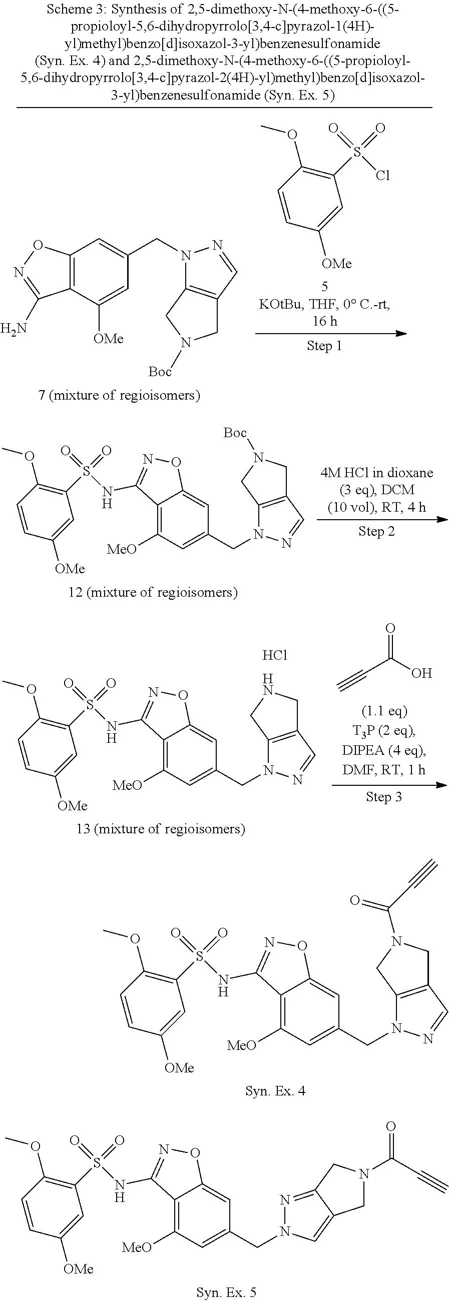

### Synthesis of tert-butyl 1-((3-((2,5-dimethoxyphenyl)sulfonamido)-4-methoxybenzo[d]isoxazol-6-yl)methyl)-4,6-dihydropyrrolo[3,4-c]pyrazole-5(1H)-carboxylate and tert-butyl 2-((3-((2,5-dimethoxyphenyl)sulfonamido)-4-methoxybenzo[d]isoxazol-6-yl)methyl)-2,6-dihydropyrrolo[3,4-c]pyrazole-5(4H)-carboxylate (12)

To a stirred solution of compound 7 (mixture of regioisomers) (750 mg, 1.945 mmol) in THF (20 mL) was added at 1M solution of KO^{t}Bu in THF (5.8 mL, 5.837 mmol) 0° C. The reaction mixture was stirred for 10 min followed by addition of 2,5-dimethoxybenzenesulfonyl chloride (0.69 g, 2.917 mmol) at 0° C. The reaction was stirred at room temperature for 16 h. After completion (monitored by TLC), the reaction mixture was quenched with ice-cold water and extracted with ethyl acetate. The organic layer was dried over anhydrous Na₂SO₄ and concentrated under reduced pressure. The crude was purified by combi flash column chromatography using 80-90% EA/Heptane to afford title compound 12 (mixture of regioisomers) (450 mg, 39.50%) as an off-white solid. TLC: 50% EA/Heptane (R_{f}: 0.5). LCMS Calculated for C27H31N5O8S: 585.19; Found: 586.2 (M+H).

### Synthesis of N-(6-((5,6-dihydropyrrolo[3,4-c]pyrazol-1(4H)-yl)methyl)-4-methoxybenzo[d]isoxazol-3-yl)-2,5-dimethoxybenzenesulfonamide hydrochloride and N-(6-((5,6-dihydropyrrolo[3,4-c]pyrazol-2(4H)-yl)methyl)-4-methoxybenzo[d]isoxazol-3-yl)-2,5-dimethoxybenzenesulfonamide hydrochloride (13)

To a stirred solution of compound 12 (450 mg, 0.768 mmol) in DCM (5 mL) was added 4M HCl in 1,4 Dioxane (4 mL) at 0° C. The reaction mixture was stirred at room temperature for 4 h. After completion (monitored by TLC), the mixture was concentrated in vacuo. The residue was washed with diethyl ether and dried in vacuo to afford the title compound 13 (mixture of regioisomers) (350 mg, 87.28%) as an off-white solid (HCl salt). TLC: 10% MeOH/DCM (R_{f}: 0.2). LCMS Calculated for C22H23N5O6S: 485.14; Found: 486.30 (M+1).

### Synthesis of 2,5-dimethoxy-N-(4-methoxy-6-((5-propioloyl-5,6-dihydropyrrolo[3,4-c]pyrazol-1(4H)-yl)methyl)benzo[d]isoxazol-3-yl)benzenesulfonamide (Syn. Ex. 4) and 2,5-dimethoxy-N-(4-methoxy-6-((5-propioloyl-5,6-dihydropyrrolo[3,4-c]pyrazol-2(4H)-yl)methyl)benzo[d]isoxazol-3-yl)benze nesulfonamide (Syn. Ex. 5)

To a stirred solution of compound 13 (200 mg, 0.383 mmol) in DMF (5 mL) was added DIPEA (0.2 mL, 1.149 mmol), propiolic acid (32 mg, 0.459 mmol) and 50% solution of T₃P in EtOAc (0.87 mL, 1.149 mmol) at 0° C. The reaction mixture was stirred at room temperature for 2 h. After completion (monitored by TLC), the mixture was quenched with ice-cold water and extracted with ethyl acetate. The organic layer was dried over anhydrous Na₂SO₄ and concentrated under reduced pressure. The crude was washed with Diethyl ether to afford a mixture of regioisomers which was further purified by chiral HPLC to get 2,5-dimethoxy-N-(4-methoxy-6((5propioloyl5,6dihydropyrrolo[3,4c]pyrazoll(4H)yl)methyl)benzo[d]isoxazol-3-yl)benzenesulfonamide (Syn. Ex. 4; 35 mg, 15.02%) as off-white and 2,5-dimethoxy-N-(4-methoxy-6-((5-propioloyl-5,6-dihydropyrrolo[3,4-c]pyrazol-2(4H)-yl)methyl)benzo[d]isoxazol-3-yl)benzenesulfonamide (Syn. Ex. 5; 15 mg) as an off-white solid. TLC: 10% MeOH/DCM (R_{f}: 0.4). (See Table 1 for analytical data).

### Examples 6-7

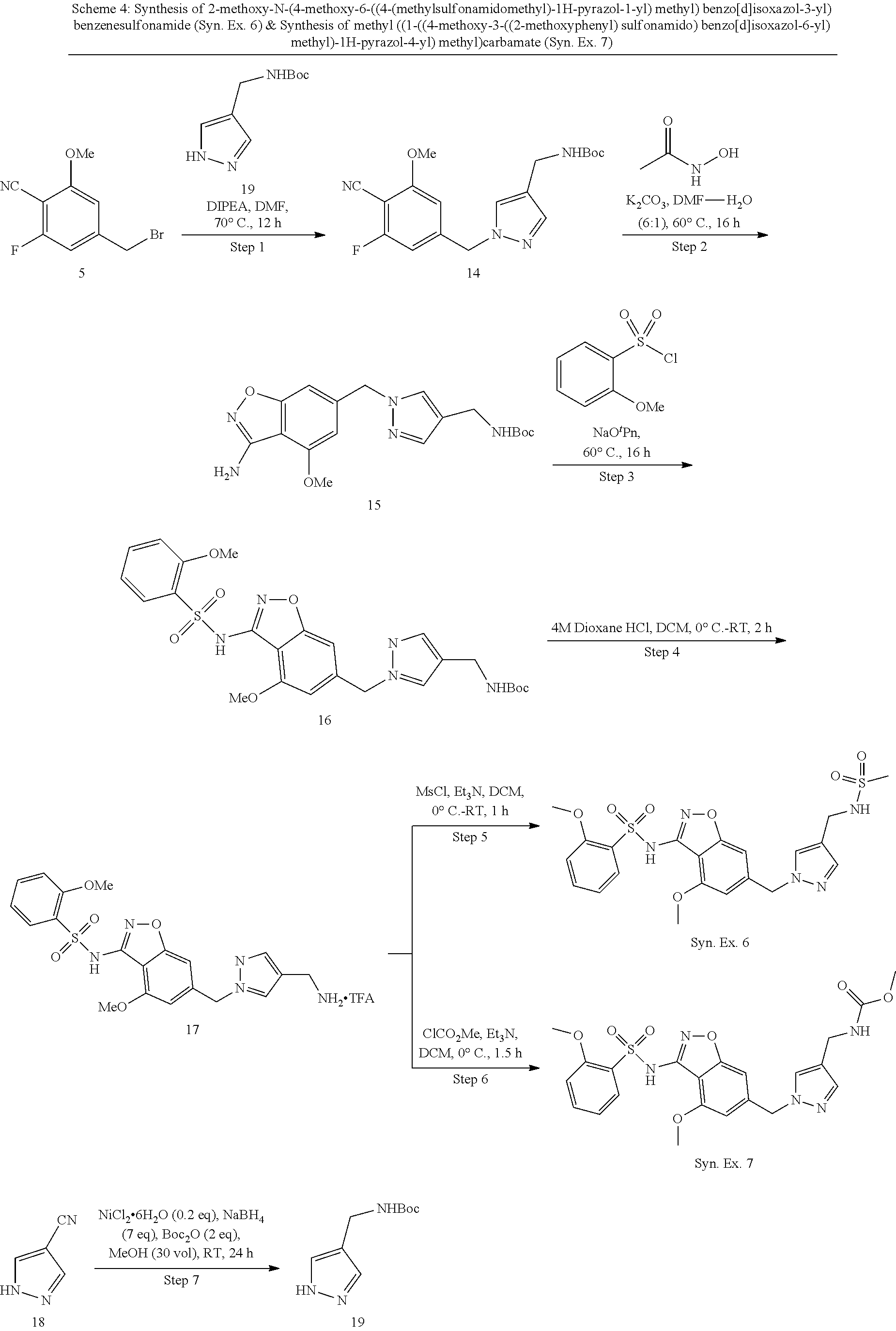

### Synthesis of tert-butyl ((1H-pyrazol-4-yl) methyl) carbamate (19)

To a stirred solution of compound 18 (5 g, 53.711 mmol) in methanol (400 mL) was added NiCl₂·6H₂O (5.01 g, 38.671 mmol) followed by addition of sodium borohydride (21.00 g, 549.5 mmol) at 0° C. The reaction mixture was stirred at room temperature for 2 h followed by addition of Di-tert-butyl dicarbonate (24 mL, 107.422). The mixture was stirred at room temperature for 48 h. After completion (monitored by TLC), the mixture was quenched with H₂O (50 mL) and evaporated under reduced pressure. The aqueous layer was extracted with EtOAc (5×30 mL). The combined organic layer was filtered through a Celite Pad, dried over anhydrous Na₂SO₄ and concentrated in vacuo to afford the title compound 19 (2.5 g, 24%) as a blue solid. TLC: 5% MeOH/DCM (R_{f}: 0.30). LCMS Calculated for C9H15N3O2: 197.12; Found: 198.20 (M+1).

### Synthesis of tert-butyl ((1-(4-cyano-3-fluoro-5-methoxybenzyl)-1H-pyrazol-4-yl) methyl) carbamate (14)

To a stirred solution of compound 5 (15 g, 61.460 mmol) in DMF (15 mL) was added DIPEA (21.46 mL, 122.92 mmol) and compound 19 (14.54 g, 73.752 mmol) at the room temperature. The resulting reaction mixture was heated at 70° C. for 12 h. After completion (monitored by TLC), the mixture was cooled to room temperature, quenched with ice-cold water and extracted with ethyl acetate. The organic layer was washed with brine, dried over anhydrous Na₂SO₄ and concentrated under reduced pressure. The crude compound was purified by Combi-flash chromatography using a gradient method of 40-70% EtOAc/Heptane to afford the title compound 14 (12 g, 33.30%) as a colourless semi solid. TLC: 60% EtOAc/Heptane (R_{f}: 0.45). LCMS Calculated for C₁₈H₂₁FN₄O₃: 360.39; Found: 361.00 (M+1).

### Synthesis of tert-butyl ((1-((3-amino-4-methoxybenzo[d]isoxazol-6-yl) methyl)-1H-pyrazol-4-yl)methyl)carbamate (15)

To a stirred solution of compound 14 (0.2 g, 0.55 mmol) in a 6:1 mixture of DMF: H₂O (7 mL) was added acetohydroxamic acid (0.112 g, 1.50 mmol) and K₂CO₃ (0.414 g, 3.0 mmol) at room temperature. The reaction mixture was allowed to stir at 60° C. for 16 h. After completion (monitored by TLC), the mixture was cooled to room temperature, quenched with ice-cold water and extracted with ethyl acetate. The organic layer was washed with brine, dried over anhydrous Na₂SO₄ and concentrated under reduced pressure. The crude compound was purified by Combi flash chromatography using a gradient method of 40-60% EtOAc/Heptane to afford the title compound 15 (0.1 g, 48.25%) as a brown semi-solid. TLC: 80% EtOAc/Heptane (R_{f}: 0.40). LCMS Calculated for C₁₈H₂₃N₅O₄: 373.41; Found: 374.05 (M+1).

### Synthesis tert-butyl ((1-((4-methoxy-3-((2-methoxyphenyl) sulfonamido) benzo[d]isoxazol-6-yl) methyl)-1H-pyrazol-4-yl) methyl) carbamate (16)

To a stirred solution of compound 15 (0.45 g, 1.20 mmol) in THF (5 mL) was added 2-methoxybenzenesulfonyl chloride (0.49 g, 2.41 mmol) followed by NaO^{t}Pn (0.396 g, 3.60 mmol). The resulting reaction mixture was allowed to stir at 60° C. for 16 h. After completion (monitored by TLC), the reaction mixture was quenched with ice cold water and extracted with ethyl acetate. The organic layer was washed with brine, dried over anhydrous Na₂SO₄, and concentrated under reduced pressure. The crude compound was purified by Combi flash chromatography using a gradient method of 60-80% ethyl acetate/Heptane to afford the title compound 16 (0.130 g, 19.9%) as a brown gummy solid. TLC: 5% MeOH/DCM (R_{f}: 0.6). LCMS Calculated for C₂₅H₂₉N₅O₇S: 543.60; Found: 542.1 (M−1).

### Synthesis of N-(6-((4-(aminomethyl)-1H-pyrazol-1-yl) methyl)-4-methoxybenzo[d]isoxazol-3-yl)-2-methoxybenzenesulfonamide hydrochloride (17)

To a stirred solution of compound 16 (0.13 g, 0.239 mmol) in DCM (3 mL) was added 4M HCl in 1,4 Dioxane (0.5 mL, 2.39 mmol) at 0° C. The reaction was allowed to stir at the room temperature for 2 h. After completion (monitored by TLC), the reaction mixture was concentrated under reduced pressure to afford the title compound 17 (0.1 g, 94.3% HCl salt) as a brown semi solid. TLC: 10% MeOH/DCM (R_{f}: 0.5). LCMS Calculated for C20H22ClN5O5S: 479.10: Found: 442.4 (M−1).

### Synthesis of 2-methoxy-N-(4-methoxy-6-((4-(methylsulfonamidomethyl)-1H-pyrazol-1-yl) methyl) benzo[d]isoxazol-3-yl) benzenesulfonamide (Syn. Ex. 6)

To a stirred solution of compound 17 (0.13 g, 0.270 mmol) in DCM (5 mL) were added Et₃N (0.11 mL, 0.812 mmol) followed by MsCl (0.037 g, 0.324 mmol) in DCM (1 mL) at 0° C. The reaction mixture was stirred at 0° C. for 1 h. After completion (monitored by TLC), the mixture was quenched with water and extracted with DCM. The organic layer was dried over anhydrous Na₂SO₄, filtered, and concentrated under reduced pressure. The crude was purified by prep-HPLC to afford title compound 2-methoxy-N-(4-methoxy-6-((4-(methylsulfonamidomethyl)-1H-pyrazol-1-yl) methyl) benzo[d]isoxazol-3-yl) benzenesulfonamide (Syn. Ex. 6; 30 mg, 21.24%) as an off-white solid. TLC: 40% EtOAc/Heptane (R_{f}: 0.5). (See Table 1 for analytical data).

### Synthesis of methyl ((1-((4-methoxy-3-((2-methoxyphenyl) sulfonamido) benzo[d]isoxazol-6-yl) methyl)-1H-pyrazol-4-yl) methyl) carbamate (Syn. Ex. 7)

To a solution of compound 17 (0.12 g, 0.250 mmol) in DCM (10 mL) was added Et₃N (0.1 mL, 0.750 mmol) followed by methyl chloroformate (0.028 g, 0.300 mmol) in DCM (1 mL) at 0° C. The reaction mixture was stirred at room temperature for 30 min. After completion (monitored by TLC), the mixture was quenched with water and extracted with DCM. The organic layer was dried over anhydrous Na₂SO₄, filtered, and concentrated under reduced pressure. The crude was purified using prep HPLC to afford title compound methyl ((1-((4-methoxy-3-((2-methoxyphenyl) sulfonamido) benzo[d]isoxazol-6-yl) methyl)-1H-pyrazol-4-yl) methyl) carbamate (Syn. Ex. 7; 25 mg, 20%) as an off-white solid. TLC: 50% EtOAc/Heptane (R_{f}: 0.5). (See Table 1 for analytical data).

### Example 8

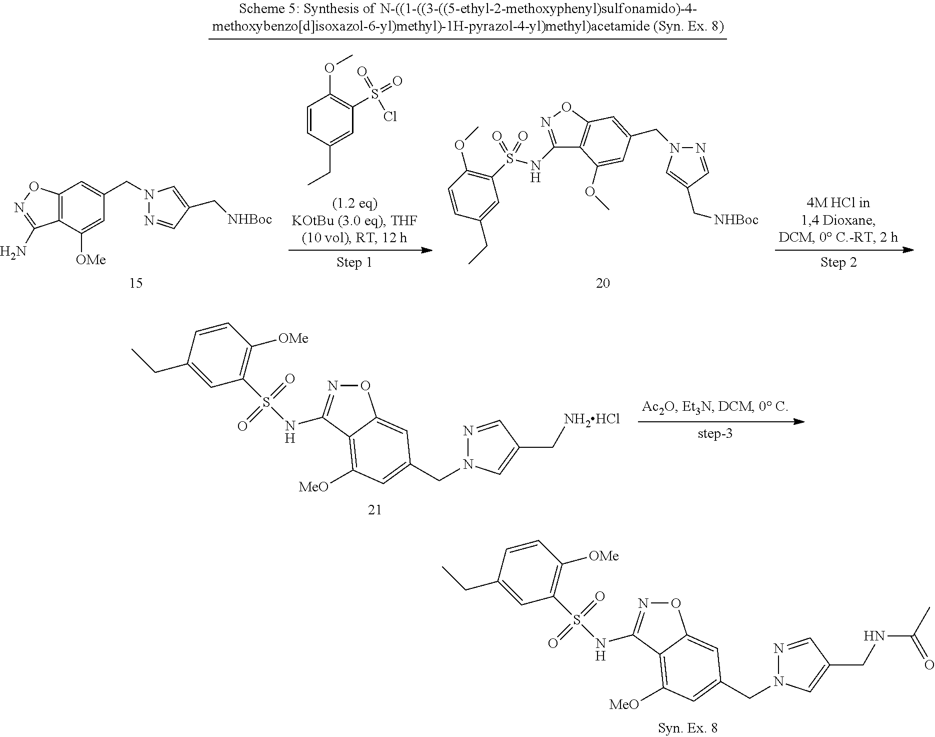

### Synthesis of tert-butyl ((1-((3-((5-ethyl-2-methoxyphenyl) sulfonamido)-4-methoxybenzo[d]isoxazol-6-yl) methyl)-1H-pyrazol-4-yl) methyl) carbamate (20)

To a stirred solution of compound 15 (0.150 g, 0.401 mmol) in THF (3 mL) was added 1M solution of tBuOK in THF (1.2 mL, 1.20 mmol) followed by 5-ethyl-2-methoxybenzenesulfonyl chloride (185 mg, 0.898 mmol) at 0° C. The reaction mixture was allowed to stir at 0° C. for 3 h. After completion (monitored by TLC), the mixture was quenched with water and extracted with ethyl acetate. The organic layer was washed with brine, dried over anhydrous Na₂SO₄ and concentrated under reduced pressure. The crude was purified by Combi flash column chromatography using a gradient method of 40-60% EtOAc/Heptane to afford the title compound 20 (130 mg, 56.76%) as an off-white solid. TLC: 80% EtOAc/Heptane (R_{f}: 0.35). LCMS Calculated for C₂₇H33N5O7S: 571.21; Found: 572.03 (M+1).

### Synthesis of N-(6-((4-(aminomethyl)-1H-pyrazol-1-yl) methyl)-4-methoxybenzo[d]isoxazol-3-yl)-5-ethyl-2-methoxybenzenesulfonamide hydrochloride (21)

To a stirred solution of compound 20 (130 mg, 0.227 mmol) in DCM (2 mL) was added 4M HCl in 1,4-dioxane (2 mL) at 0° C. The reaction mixture was allowed to stir at room temperature for 2 h. After completion (monitored by TLC), the mixture was evaporated under reduced pressure. The crude was triturated with DCM/Heptane to afford the title compound 21 (100 mg, salt) as an off-white solid. TLC: 5% MeOH/DCM (R_{f}: 0.3). LCMS Calculated for C₂₂H25N5O5S: 471.16; Found: 472.30.

### Synthesis of N-((1-((3-((5-ethyl-2-methoxyphenyl) sulfonamido)-4-methoxybenzo[d]isoxazol-6-yl) methyl)-1H-pyrazol-4-yl) methyl) acetamide (Syn. Ex. 8)

To a stirred solution of compound 21 (100 mg, 0.196 mmol) in DCM (3 mL) was added triethylamine (0.082 mL, 0.590 mmol) at 0° C. The reaction mixture was stirred for 10 min. followed by addition of Ac₂O (0.022 mL, 0.235 mmol) in DCM (1 mL) at 0° C. The reaction mixture was stirred at room temperature for 1 h. After completion (monitored by TLC), the mixture was quenched with water and extracted with DCM. The organic layer was dried over anhydrous Na₂SO₄, and concentrated under reduced pressure to get crude. The crude was purified using prep. HPLC to afford N-((1-((3-((5-ethyl-2-methoxyphenyl) sulfonamido)-4-methoxybenzo[d]isoxazol-6-yl)methyl)-1H-pyrazol-4-yl)methyl) acetamide (Syn. Ex. 8; 45 mg, 44.55%) as an off-white solid. TLC: 10% MeOH/DCM (R_{f}: 0.5). (See Table 1 for analytical data).

### Example 9

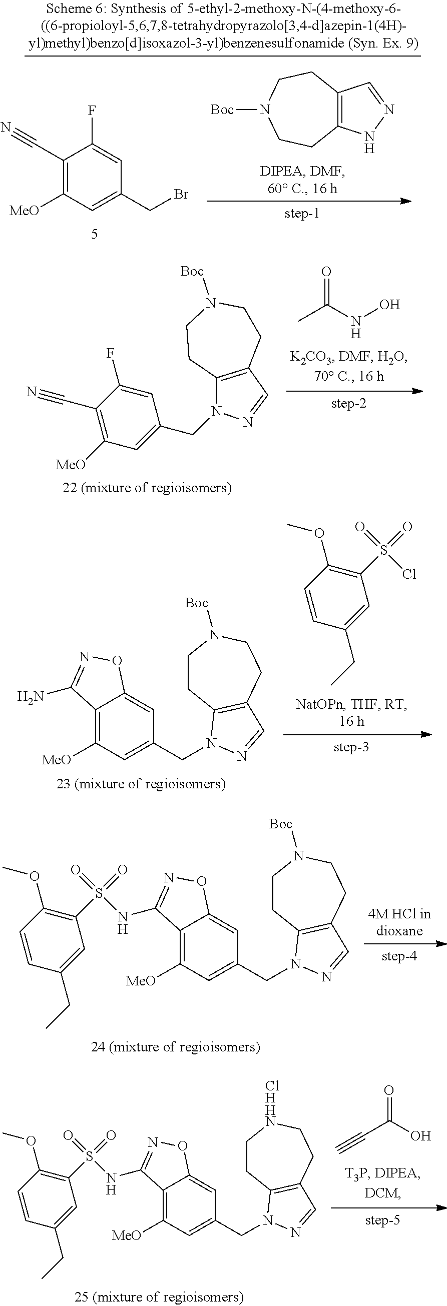

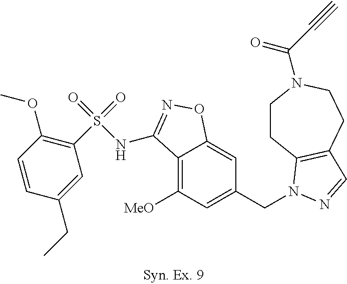 [SMILES: C#CC(=O)N1CCc2cnn(Cc3cc(OC)c4c(NS(=O)(=O)c5cc(CC)ccc5OC)noc4c3)c2CC1]

### Synthesis of tert-butyl 1-(4-cyano-3-fluoro-5-methoxybenzyl)-4,5,7,8-tetrahydropyrazolo[3,4-d]azepine-6(1H)-carboxylate and tert-butyl 2-(4-cyano-3-fluoro-5-methoxybenzyl)-4,5,7,8-tetrahydropyrazolo[3,4-d]azepine-6(2H)-carboxylate (22)

To a stirred solution of compound 5 (0.75 g, 3.073 mmol) in DMF (7 mL) was added DIPEA (1.6 mL, 9.219 mmol) followed by tert-butyl 4,5,7,8-tetrahydropyrazolo[3,4-d]azepine-6(1H)-carboxylate (0.729 g, 3.073 mmol). The reaction mixture was stirred at 60° C. for 16 h. After completion (monitored by TLC), the mixture was quenched with water and extracted with ethyl acetate. The organic layer was dried over anhydrous Na₂SO₄ and concentrated under reduced pressure to afford crude compound 22 (1.2 g, crude) as a brown liquid. TLC: 40% EtOAc/Heptane (R_{f}: 0.4). LCMS Calculated for C21H25FN4O3: 400.19; Found: 400.30

### Synthesis of tert-butyl 1-((3-amino-4-methoxybenzo[d]isoxazol-6-yl) methyl)-4,5,7,8-tetrahydropyrazolo[3,4-d]azepine-6(1H)-carboxylate and tert-butyl 2-((3-amino-4-methoxybenzo[d]isoxazol-6-yl)methyl)-4,5,7,8-tetrahydropyrazolo[3,4-d]azepine-6(2H)-carboxylate (23)

To a solution of compound 22 (1.2 g, 2.996 mmol) in a mixture of DMF: H₂O (48 mL, 6:1 ratios) was added K₂CO₃ (2.48 g, 17.979 mmol) and N-hydroxy acetamide (0.674 g, 8.988 mmol). The reaction mixture was stirred at 70° C. for 16 h. After completion (monitored by TLC), the reaction mixture was quenched with water and extracted with ethyl acetate. The organic layer was dried over anhydrous Na₂SO₄, and concentrated under reduced pressure to afford the title compound 23 (750 mg, 60.53%) as a yellow solid. TLC: 40% EtOAc/Heptane (R_{f}: 0.4). LCMS Calculated for C21H27N5O4: 413.21; Found: 414.5 (M+1).

### Synthesis of tert-butyl 1-((3-((5-ethyl-2-methoxyphenyl)sulfonamido)-4-methoxybenzo[d]isoxazol-6-yl)methyl)-4,5,7,8-tetrahydropyrazolo[3,4-d]azepine-6(1H)-carboxylate and tert-butyl 2-((3-((5-ethyl-2-methoxyphenyl)sulfonamido)-4-methoxybenzo[d]isoxazol-6-yl)methyl)-4,5,7,8-tetrahydropyrazolo[3,4-d]azepine-6(2H)-carboxylate (24)

To a stirred solution of compound 23 (750 mg, 1.813 mmol) in THF (8 mL) was added NaO^{t}Bu (0.87 g, 9.069 mmol) followed by 5-ethyl-2-methoxybenzenesulfonyl chloride (0.510 g, 2.175 mmol) at 0° C. The reaction mixture was stirred at room temperature for 16 h. After completion (monitored by TLC), the reaction mixture was quenched with water and extracted with ethyl acetate. The organic layer was dried over anhydrous Na₂SO₄, and concentrated under reduced pressure to offer the title compound 24 (1.1 g, 99.18%) as a brown gummy oil. The crude was carried forward to the next step without further purification. TLC: 40% EtOAc/Heptane (R_{f}: 0.4). LCMS Calculated for C30H37N5O7S: 611.24; Found: 612.5 (M+1).

### Synthesis of 5-ethyl-2-methoxy-N-(4-methoxy-6-((5,6,7,8-tetrahydropyrazolo[3,4-d]azepin-1(4H)-yl)methyl)benzo[d]isoxazol-3-yl)benzenesulfonamide hydrochloride and 5-ethyl-2-methoxy-N-(4-methoxy-6-((5,6,7,8-tetrahydropyrazolo[3,4-d]azepin-2(4H)-yl)methyl)benzo[d]isoxazol-3-yl)benzenesulfonamide hydrochloride (25)

To a solution of compound 24 (1.0 g, 1.63 mmol) in DCM (10 mL) was added 4M HCl in 1,4 Dioxane (5 mL) at 0° C. and the reaction mixture was stirred at room temperature for 4 h. After completion (reaction monitored by TLC), the mixture was concentrated under reduced pressure. The residue was washed diethyl ether 3 times and dried in vacuo to afford the title compound 25 (750 mg, HCl salt) as an off-white solid. The crude has been carried forward in next reaction. It was taken to the next step with no further purification. TLC: 40% EtOAc/Heptane (R_{f}: 0.4). LCMS Calculated for C₂₅H29N5O5S: 511.19; Found: 512.30.

### Synthesis of 5-ethyl-2-methoxy-N-(4-methoxy-6-((6-propioloyl-5,6,7,8-tetrahydropyrazolo[3,4-d]azepin-1(4H)-yl)methyl)benzo[d]isoxazol-3-yl)benzenesulfonamide (Syn. Ex. 9)

To a stirred solution of compound 25 (0.750 g, 1.368 mmol) and prop-2-ynoic acid (0.143 g, 2.052 mmol) in DMF (7.5 mL) were added DIPEA (0.716 mL, 4.104 mmol) and 50% solution of T₃P in EtOAc (5.22 mL, 8.208 mmol) at 0° C. The reaction mixture was stirred at room temperature for 2 h. After completion (monitored by TLC), the mixture was quenched with water and extracted with DCM. The organic layer was dried over anhydrous Na₂SO₄, and concentrated under reduced pressure. The crude was purified by combi-flash chromatography (5% MeOH in DCM) followed by prep-HPLC to get the desired compound as a mixture of regioisomers. The desired regioisomer (Syn. Ex. 9) was obtained by subjecting this mixture to prep-SFC to afford 5-ethyl-2-methoxy-N-(4-methoxy-6-((6-propioloyl-5,6,7,8-tetrahydropyrazolo[3,4-d]azepin-1(4H)-yl)methyl)benzo[d]isoxazol-3-yl) benzenesulfonamide (Syn. Ex. 9; 55 mg, 7.13%) as an off-white solid. The other regioisomer was not isolated. TLC: 10% MeOH/DCM (R_{f}: 0.5). (See analytical data in Table 1).

### Example 10

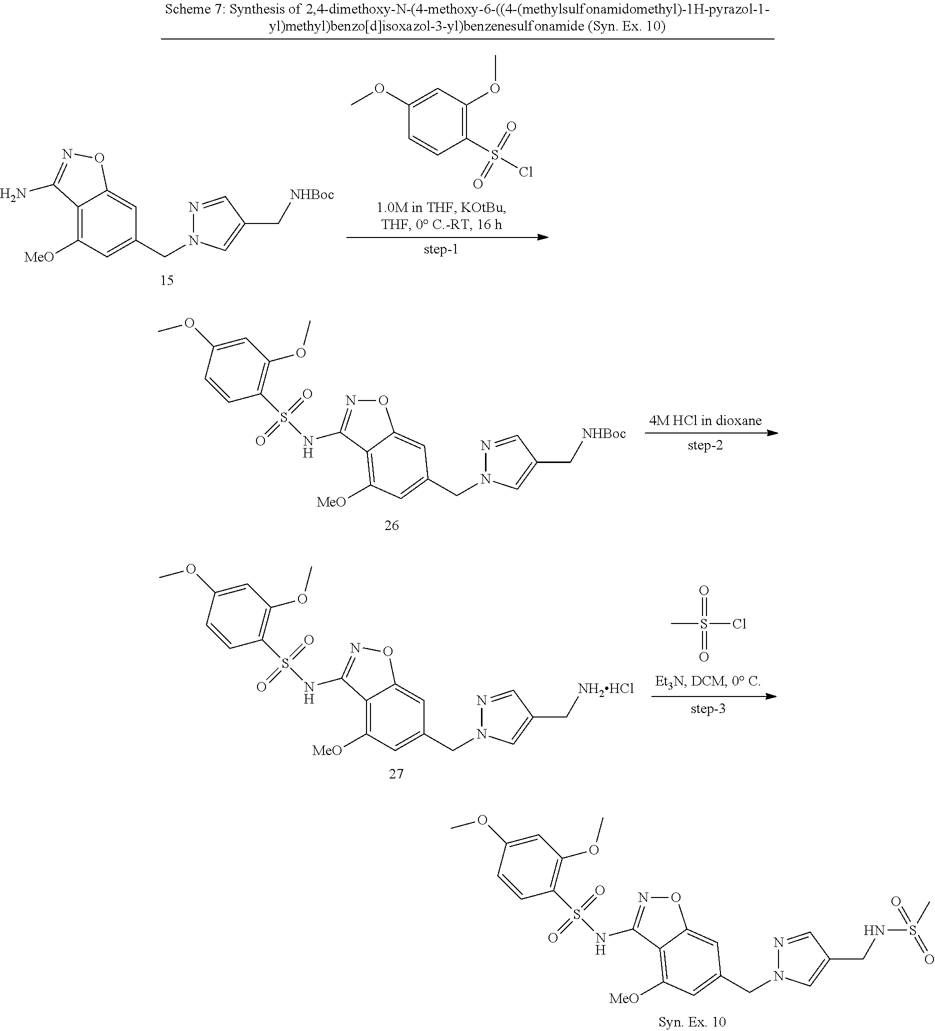

### Synthesis of tert-butyl ((1-((3-((2,4-dimethoxyphenyl)sulfonamido)-4-methoxybenzo[d]isoxazol-6-yl)methyl)-1H-pyrazol-4-yl)methyl)carbamate (26)

To a stirred solution of compound 15 (500 mg, 1.339 mmol) in THF (15 mL) was added 1M solution of KO^{t}Bu solution in THF (4 mL, 4.017 mmol) at 0° C. The reaction mixture was stirred for 10 min at 0° C. followed by addition of 2,4-dimethoxybenzenesulfonyl chloride (0.475 g, 2.008 mmol) in THF (1 mL). The reaction was stirred at room temperature for 16 h. After completion (monitored by TLC), the mixture was quenched with 1N HCl and extracted with ethyl acetate. The organic layer was dried over anhydrous Na₂SO₄, and concentrated under reduced pressure. The crude was purified by combi flash chromatography using (neutral alumina) gradient 30-50% EA/Heptane to afford the title compound 26 (140 mg, 18.22%) as a pale-yellow solid. TLC: 10% MeOH/DCM (R_{f}: 0.6). LCMS Calculated for C₂₆H₃₁N₅O₈S: 573.62; Found: 574.06 (M+1).

### Synthesis of N-(6-((4-(amino methyl)-1H-pyrazol-1-yl) methyl)-4-methoxybenzo[d]isoxazol-3-yl)-2,4-dimethoxybenzenesulfonamide hydrochloride (27)

To a stirred solution of compound 26 (140 mg, 0.274 mmol) in DCM (3 mL) was added 4M HCl in 1,4 Dioxane (2 mL) at 0° C. The reaction mixture was stirred at room temperature for 4 h. After completion (monitored by TLC), the mixture was concentrated under reduced pressure. The residue was washed with diethyl ether to afford the title compound 27 (110 mg, 74.24%) as an off-white solid (HCl salt). TLC: 10% MeOH/DCM (R_{f}: 0.6). LCMS Calculated for C₂₁H23N5O6S: 473.14; found: 474.20.

### Synthesis of 2,4-dimethoxy-N-(4-methoxy-6-((4-(methylsulfonamidomethyl)-1H-pyrazol-1-yl)methyl)benzo[d]isoxazol-3-yl)benzenesulfonamide (Syn. Ex. 10)

To a stirred solution of compound 27 (110 mg, 0.2157 mmol) in DCM (3 mL) was added triethylamine (0.09 mL, 0.647 mmol) followed by methanesulfonyl chloride (0.029 g, 0.2588 mmol) in DCM (1 mL) at 0° C. The reaction was mixture was stirred at room temperature for 15 min. After completion (monitored by TLC), the mixture was quenched with water and extracted with DCM. The organic layer was dried over anhydrous Na₂SO₄, and concentrated under reduced pressure. The crude was purified by using prep-HPLC to afford 2,4-dimethoxy-N-(4-methoxy-6-((4-(methylsulfonamidomethyl)-1H-pyrazol-1-yl)methyl)benzo[d]isoxazol-3-yl)benzenesulfonamide (Syn. Ex. 10; 29 mg, 24.57%) as an off-white solid. TLC: 10% MeOH/DCM (R_{f}: 0.5). (See Table 1 for analytical data).

### Examples 11-13

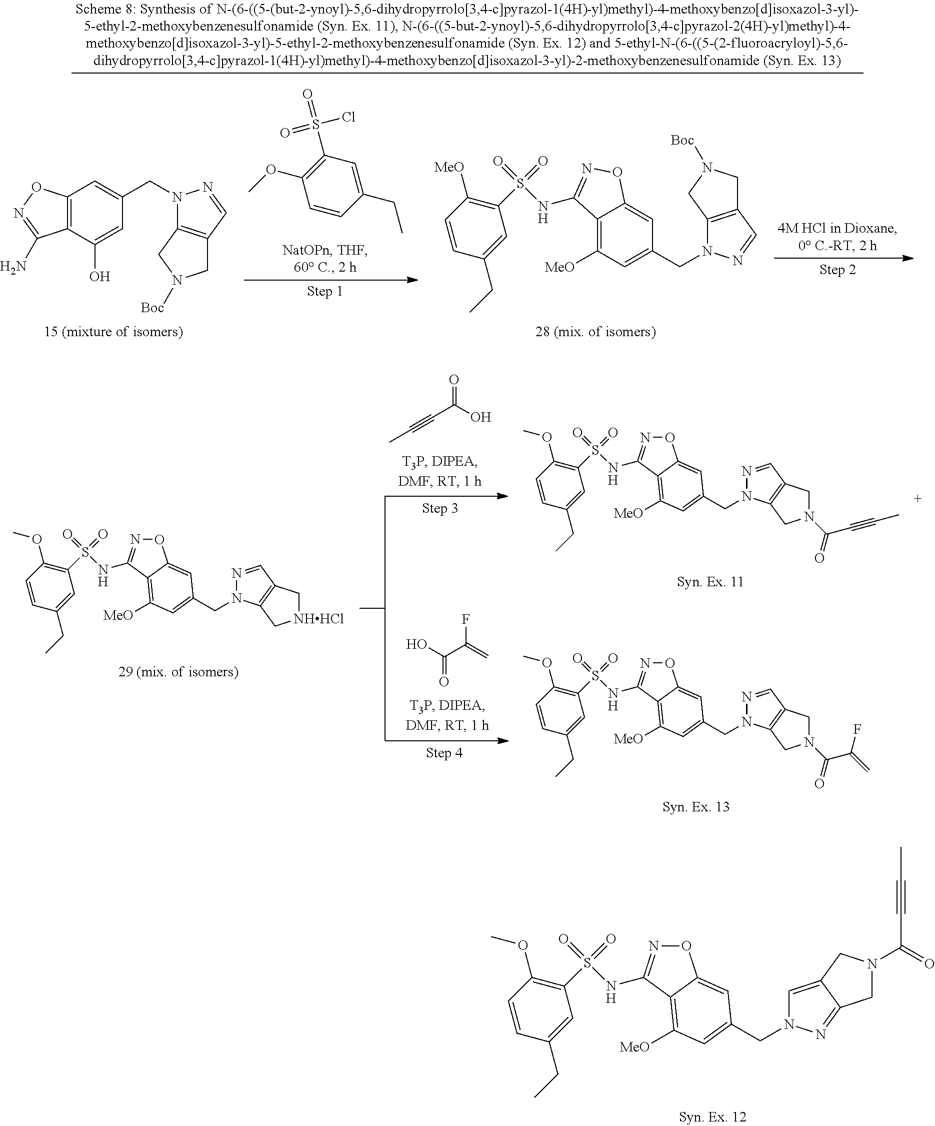

### Synthesis of tert-butyl 1-((3-((5-ethyl-2-methoxyphenyl) sulfonamido)-4-methoxybenzo[d]isoxazol-6-yl)methyl)-4,6-dihydropyrrolo[3,4-c]pyrazole-5(1H)-carboxylate and tert-butyl 2-((3-((5-ethyl-2-methoxyphenyl)sulfonamido)-4-methoxybenzo[d]isoxazol-6-yl)methyl)-2,6-dihydropyrrolo[3,4-c]pyrazole-5(4H)-carboxylate (28)

To a stirred solution of compound 15 (mixture of isomers) (1.3 g, 3.372 mmol) in THF (20 mL) was added NaO^{t}Pn (2.22 g, 20.237 mmol) followed by 5-ethyl-2-methoxybenzenesulfonyl chloride (1.58 g, 6.744 mmol) at 0° C. The reaction was allowed to stir at 60° C. for 16 h. After completion (monitored by TLC), the mixture was quenched with water and extracted with EtOAc. The organic layer was washed with brine, dried over anhydrous Na₂SO₄, and concentrated under reduced pressure. The crude was purified by Combi flash using a gradient method of 20-60% EtOAc/Heptane to afford the title compound 28 (mixture of isomers) (1.0 g, 51.02%) as a pale-yellow solid. TLC: 100% EtOAc (R_{f}: 0.5). LCMS Calculated for: C28H33N5O7S: 583.21; Found: 584.5 (M+1).

### Synthesis of N-(6-((5,6-dihydropyrrolo[3,4-c]pyrazol-1(4H)-yl) methyl)-4-methoxybenzo[d]isoxazol-3-yl)-5-ethyl-2-methoxybenzenesulfonamide hydrochloride and N-(6-((5,6-dihydropyrrolo[3,4-c]pyrazol-2(4H)-yl)methyl)-4-methoxybenzo[d]isoxazol-3-yl)-5-ethyl-2-methoxybenzenesulfonamide hydrochloride (29)

To a stirred solution of compound 28 (mixture of isomers) (1 g, 1.713 mmol) in DCM (5 mL) was added 4M HCl in 1,4 Dioxane (4.28 mL, 17.133 mmol) at 0° C. The reaction was allowed to stir at room temperature for 2 h. After completion (monitored by TLC), the mixture was concentrated under reduced pressure and triturated with Et₂O to afford the title compound 29 (mixture of isomers) (750 mg, HCl salt) as a gummy off-white solid. TLC: 5% MeOH/DCM (R_{f}: 0.2). LCMS Calculated for: C₂₃H25N5O5S: 483.16; Found: 484.20.

### Synthesis of N-(6-((5-(but-2-ynoyl)-5,6-dihydropyrrolo[3,4-c]pyrazol-1(4H)-yl)methyl)-4-methoxybenzo[d]isoxazol-3-yl)-5-ethyl-2-methoxybenzenesulfonamide (Syn. Ex. 11) and N-(6-((5-(but-2-ynoyl)-5,6-dihydropyrrolo[3,4-c]pyrazol-2(4H)-yl)methyl)-4-methoxybenzo[d]isoxazol-3-yl)-5-ethyl-2-methoxybenzenesulfonamide (Syn. Ex. 12)

To a stirred solution of compound 29 (mix. of isomers) (350 mg, 0.673 mmol) in DMF (3 mL) was added DIPEA (0.352 mL, 2.019 mmol), but-2-ynoic acid (67 mg, 0.807 mmol) followed by 50% solution of T₃P in EtOAc (1.28 mL, 2.019 mmol) at 0° C. The reaction mixture was allowed to stir at room temperature for 16 h. After completion (monitored by TLC), the mixture was concentrated under reduced pressure. The crude was purified by Chiral HPLC purification to afford the 2 isomers N-(6-((5-(but-2-ynoyl)-5,6-dihydropyrrolo[3,4-c]pyrazol-1(4H)-yl)methyl)-4-methoxybenzo[d]isoxazol-3-yl)-5-ethyl-2-methoxybenzenesulfonamide (Syn. Ex. 11; 45 mg, 11.31%) as an off white solid and N-(6-((5-(but-2-ynoyl)-5,6-dihydropyrrolo[3,4-c]pyrazol-2(4H)-yl)methyl)-4-methoxybenzo[d]isoxazol-3-yl)-5-ethyl-2-methoxybenzenesulfonamide (Syn. Ex. 12; 14 mg, 5.31% yield) as an off white solid. TLC: 5% MeOH/DCM (R_{f}: 0.5). (See Table 1 for analytical data).

### Synthesis of 5-ethyl-N-(6-((5-(2-fluoroacryloyl)-5,6-dihydropyrrolo[3,4-c]pyrazol-1(4H)-yl)methyl)-4-methoxybenzo[d]isoxazol-3-yl)-2-methoxybenzenesulfonamide (Syn. Ex. 13)

To a stirred solution of compound 29 (mix. of isomers) (200 mg, 0.384 mmol) in DMF (3 mL) was added DIPEA (0.2 mL, 1.153 mmol), 2-fluoroacrylic acid (41 mg, 0.460 mmol) followed by 50% solution of T₃P in EtOAc (0.733 mL, 1.153 mmol) at 0° C. The reaction mixture was allowed to stir at room temperature for 1 h. After completion (monitored by TLC), the reaction mixture was concentrated under reduced pressure. The crude was purified by Chiral HPLC purification to afford the title compound N-(6-((5-(but-2-ynoyl)-5,6-dihydropyrrolo[3,4-c]pyrazol-1(4H)-yl)methyl)-4-methoxybenzo[d]isoxazol-3-yl)-5-ethyl-2-methoxybenzenesulfonamide (Syn. Ex. 13; 15 mg, 6.52% yield) as an off white solid. TLC: 5% MeOH/DCM (R_{f}: 0.5) (See Table 1 for analytical data). The other isomer was not isolated.

### Examples 14-18

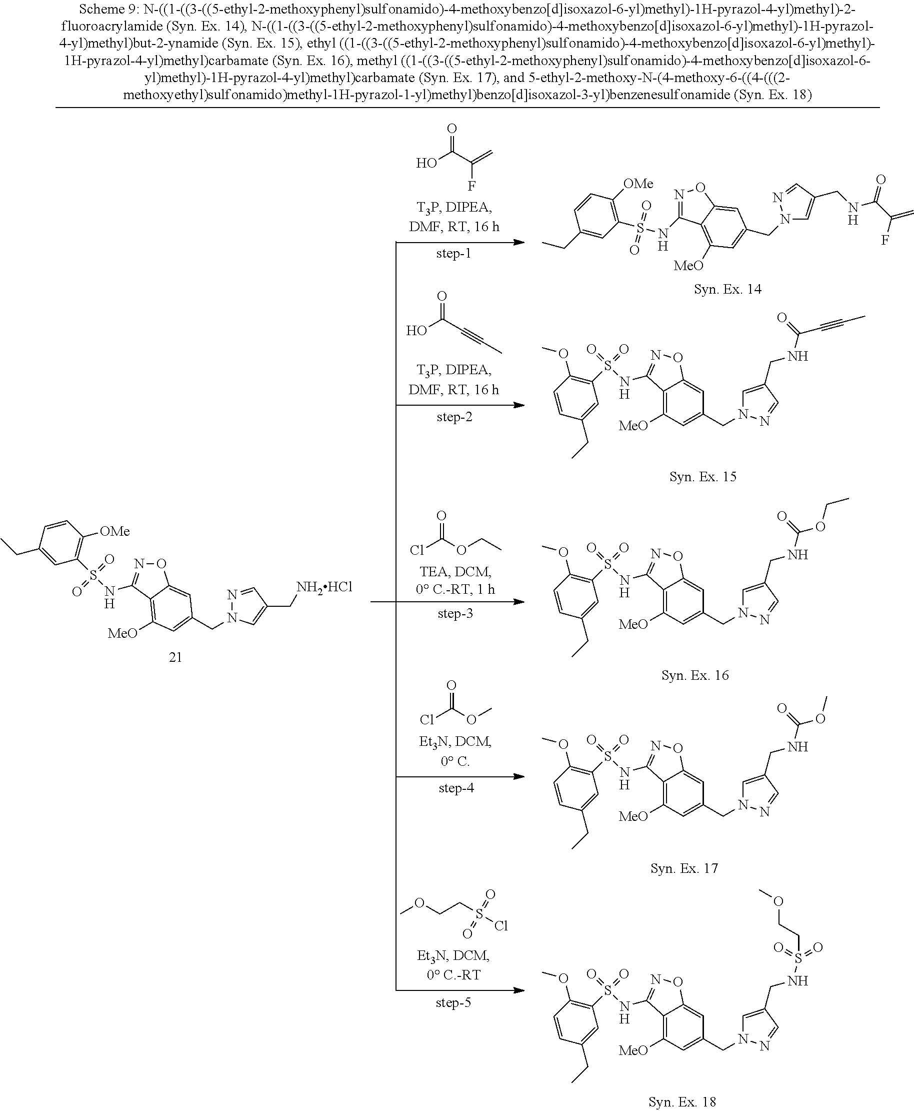

### Synthesis of N-((1-((3-((5-ethyl-2-methoxyphenyl)sulfonamido)-4-methoxybenzo[d]isoxazol-6-yl)methyl)-1H-pyrazol-4-yl)methyl)-2-fluoroacrylamide (Syn. Ex. 14)

To a stirred solution of compound 21 (0.12 g, 0.236 mmol) in DMF (2 mL) was added 2-fluoroacrylic acid (0.042 g, 0.472 mmol) and DIPEA (0.20 mL, 1.181 mmol) at 0° C. The reaction mixture was stirred at 0° C. for 10 min followed by the addition of 50% solution of T₃P in EtOAc (0.375 mL, 0.590 mmol) at 0° C. The mixture was allowed to stir at room temperature for 3 h. After completion (monitored by TLC), the mixture was quenched with ice water (10 mL). The solid precipitate was filtered, washed with heptane, and dried under reduced pressure. The crude was purified by prep HPLC to afford the title compound N-((1-((3-((5-ethyl-2-methoxyphenyl)sulfonamido)-4-methoxybenzo[d]isoxazol-6-yl)methyl)-1H-pyrazol-4-yl)methyl)-2-fluoroacrylamide (Syn. Ex. 14; 3.8 mg, 2.96%) as an off-white solid. (See Table 1 for analytical data).

### Synthesis of N-((1-((3-((5-ethyl-2-methoxyphenyl)sulfonamido)-4-methoxybenzo[d]isoxazol-6-yl)methyl)-1H-pyrazol-4-yl)methyl)but-2-ynamide (Syn. Ex. 15)

To a stirred solution of compound 21 (0.12 g, 0.236 mmol) in DMF (2 mL) was added but-2-ynoic acid (0.039 g, 0.472 mmol) and DIPEA (0.20, 1.181 mmol) at 0° C. The reaction mixture was stirred for 10 min followed by the addition of 50% solution of T₃P in EtOAc (0.36 mL, 0.590 mmol) at 0° C. The mixture was allowed to stir at room temperature for 3 h. After completion (monitored by TLC), the mixture was quenched with ice cold water (20 mL). The solid precipitate was filtered, washed with heptane, and dried under reduced pressure. The crude was purified by prep HPLC to afford title compound N-((1-((3-((5-ethyl-2-methoxyphenyl)sulfonamido)-4-methoxybenzo[d]isoxazol-6-yl)methyl)-1H-pyrazol-4-yl)methyl)but-2-ynamide (Syn. Ex. 15; 5.4 mg, 4.28%) as an off-white solid. (See Table 1 for analytical data)

### Synthesis of ethyl ((1-((3-((5-ethyl-2-methoxyphenyl)sulfonamido)-4-methoxybenzo[d]isoxazol-6-yl)methyl)-1H-pyrazol-4-yl)methyl)carbamate (Syn. Ex. 16)

To a stirred solution of compound 21 (150 mg, 0.295 mmol) in DCM (2 mL) was added TEA (0.12 mL, 0.885 mmol) followed by ethyl chloroformate (48 mg, 0.442 mmol) in DCM (1 mL) at 0° C. The reaction mixture was allowed to stir at 0° C. for 1 h. After completion (monitored by TLC and LCMS), the mixture was quenched with ice-water (20 mL) and extracted with DCM (30 mL×3). The organic layer was dried over anhydrous Na₂SO₄ and concentrated under reduced pressure to get the crude which was purified using prep-HPLC to afford ethyl ((1-((3-((5-ethyl-2-methoxyphenyl)sulfonamido)-4-methoxybenzo[d]isoxazol-6-yl)methyl)-1H-pyrazol-4-yl)methyl)carbamate (Syn. Ex. 16; 14 mg, 8.75%) as an off-white solid. (See Table 1 for analytical data).

### Synthesis of methyl ((1-((3-((5-ethyl-2-methoxyphenyl)sulfonamido)-4-methoxybenzo[d]isoxazol-6-yl)methyl)-1H-pyrazol-4-yl)methyl)carbamate (Syn. Ex. 17)

To a stirred solution of compound 21 (120 mg, 0.236 mmol) in DCM (2 mL) was added TEA (0.09 mL, 0.708 mmol) followed by methyl chloroformate (26 mg, 0.283 mmol) in DCM (1 mL) at 0° C. The reaction mixture was allowed to stir at 0° C. for 1 h. After completion (monitored by TLC and LCMS), the reaction mixture was quenched with ice cold water (30 mL) and extracted with ethyl acetate (100 mL×2). The organic layer was dried over anhydrous Na₂SO₄ and concentrated under reduced pressure to get the crude which was purified using prep-HPLC to afford methyl ((1-((3-((5-ethyl-2-methoxyphenyl)sulfonamido)-4-methoxybenzo[d]isoxazol-6-yl)methyl)-1H-pyrazol-4-yl)methyl)carbamate (Syn. Ex. 17; 9 mg, 6.834%) as an off-white solid. (See Table 1 for analytical data).

### Synthesis of 5-ethyl-2-methoxy-N-(4-methoxy-6-((4-(((2-methoxyethyl)sulfonamido)methyl)-1H-pyrazol-1-yl)methyl)benzo[d]isoxazol-3-yl)benzenesulfonamide (Syn. Ex. 18)

To a stirred solution of compound 21 (120 mg, 0.236 mmol) in DCM (2 mL) was added TEA (0.09 mL, 0.708 mmol) followed by 2-methoxyethane-1-sulfonyl chloride (44 mg, 0.283 mmol) in DCM (1 mL) at 0° C. The reaction mixture was allowed to stir at 0° C. to room temperature for 16 h. After completion (monitored by TLC and LCMS), the mixture was quenched with 1N HCl (10 mL) and extracted with EtOAc (2×50 mL). The combined organic layer was washed with brine and dried over anhydrous Na₂SO₄ and concentrated under reduced pressure. The crude was purified by prep-HPLC purification to afford 5-ethyl-2-methoxy-N-(4-methoxy-6-((4-(((2-methoxyethyl)sulfonamido)methyl)-1H-pyrazol-1-yl)methyl)benzo[d]isoxazol-3-yl)benzenesulfonamide (Syn. Ex. 18; 4.5 mg, 3.04%) as an off-white solid. (See Table 1 for analytical data).

### Examples 19-20

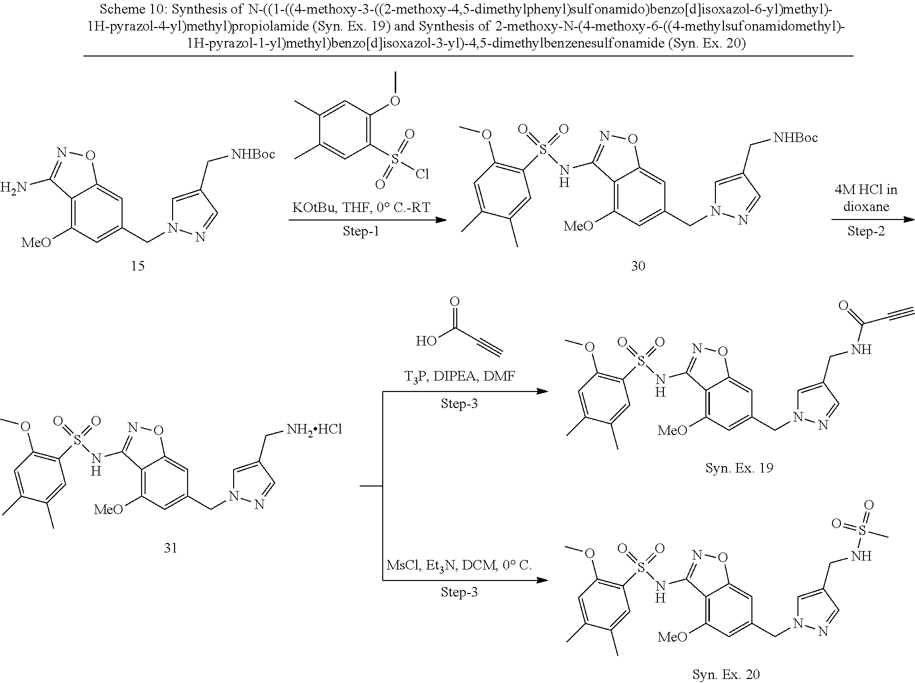

### Synthesis of tert-butyl ((1-((4-methoxy-3-((2-methoxy-4,5-dimethylphenyl) sulfonamido) benzo[d]isoxazol-6-yl) methyl)-1H-pyrazol-4-yl) methyl) carbamate (30)

To a stirred solution of compound 15 (1.0 g, 2.678 mmol) in THF (10 mL) was added 1.0 M KOtBu in THF (8.0 mL, 8.034 mmol) at 0° C. The reaction mixture was stirred at 0° C. for 10 min followed by the addition of 2-methoxy-4,5-dimethylbenzenesulfonyl chloride (0.628 g, 3.213 mmol). The mixture was stirred at room temperature for 16 h. After completion (monitored by TLC), the mixture was quenched with water (50 mL) and extracted with 10% MeOH in DCM (3×50 mL). The organic layer was dried over anhydrous Na₂SO₄, filtered and concentrated under reduced pressure. The crude was purified by combi flash column chromatography (neutral silica gel) using 80-90% EtOAc in Heptane to afford title compound 30 (0.23 g, 15.03%) as an off-white solid. LCMS calculated for C₂₇H33N5O7S: 571.21; found: 572.46 (M+1).

### Synthesis of N-(6-((4-(aminomethyl)-1H-pyrazol-1-yl)methyl)-4-methoxybenzo[d]isoxazol-3-yl)-2-methoxy-4,5-dimethylbenzenesulfonamide hydrochloride (31)

To a solution of compound 30 (0.31 g, 0.542 mmol) in dioxane (3 mL) was added 4M HCl in dioxane (2 mL) at 0° C. The reaction mixture was stirred at room temperature for 4 h. After completion (monitored by TLC), the mixture was concentrated under reduced pressure. The residue was triturated with Et₂O (3×20 mL) and dried under reduced pressure to afford the title compound 31 (0.28 g, HCl salt) as an off-white solid. LCMS calculated for C22H26ClN5O5S: 471.16; found: 472.42 (M+1).

### Synthesis of N-((1-((4-methoxy-3-((2-methoxy-4,5-dimethylphenyl)sulfonamido)benzo[d]isoxazol-6-yl)methyl)-1H-pyrazol-4-yl)methyl)propiolamide (Syn. Ex. 19)

To a stirred solution of compound 31 (0.10 g, 0.196 mmol) in DMF (1 mL) was added DIPEA (0.10 mL, 0.590 mmol), propiolic acid (0.027 g, 0.393 mmol) and 50% solution of T₃P in EtOAc (0.373 mL, 0.588 mmol) at 0° C. The reaction mixture was stirred at room temperature for 4 h. After completion (monitored by TLC), the mixture was quenched with water (20 mL) and extracted with EtOAc (3×20 mL). The combined organic layer was washed with brine (10 mL), dried over anhydrous Na₂SO₄, and concentrated under reduced pressure. The crude was purified by prep HPLC to afford the title compound N-((1-((4-methoxy-3-((2-methoxy-4,5-dimethylphenyl)sulfonamido)benzo[d]isoxazol-6-yl)methyl)-1H-pyrazol-4-yl)methyl)propiolamide (Syn. Ex. 19; 24 mg, 23.30%) as an off-white solid. (See Table 1 for analytical data).

### Synthesis of 2-methoxy-N-(4-methoxy-6-((4-(methylsulfonamidomethyl)-1H-pyrazol-1-yl) methyl) benzo[d]isoxazol-3-yl)-4,5-dimethylbenzenesulfonamide (Syn. Ex. 20)

To a stirred solution of compound 31 (0.10 g, 0.196 mmol) in DCM (2 mL) was added Et₃N (0.08 mL, 0.588 mmol) and methane sulfonyl chloride (0.020 g, 0.176 mmol) in DCM (1 mL) at 0° C. The reaction mixture was stirred at room temperature for 2 h. After completion (monitored by TLC), the mixture was quenched with water (20 mL) and extracted with DCM (3×20 mL). The combined organic layer was washed with brine (10 mL), dried over anhydrous Na₂SO₄, and concentrated under reduced pressure. The crude was purified by prep HPLC to afford the title compound 2-methoxy-N-(4-methoxy-6-((4-(methylsulfonamidomethyl)-1H-pyrazol-1-yl)methyl)benzo[d]isoxazol-3-yl)-4,5-dimethylbenzenesulfonamide (Syn. Ex. 20; 25 mg, 23.14% Yield) as an off-white solid. (See Table 1 for analytical data)

### Examples 21-22

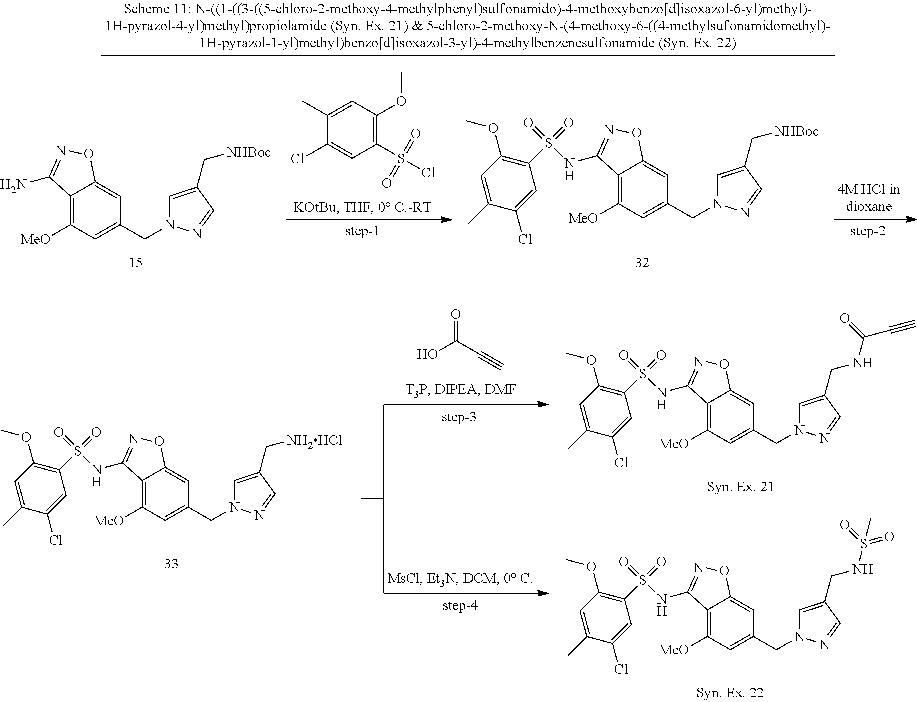

### Synthesis of tert-butyl ((1-((3-((5-chloro-2-methoxy-4-methylphenyl)sulfonamido)-4-methoxybenzo[d]isoxazol-6-yl)methyl)-1H-pyrazol-4-yl)methyl)carbamate (32)

To a stirred solution of compound 15 (0.50 g, 1.339 mmol) in THF (5 mL) was added 1.0 M solution of KO^{t}Bu in THF (4.45 mL, 4.017 mmol) at 0° C. The reaction mixture was stirred for 10 min followed by the addition of 5-chloro-2-methoxy-4-methyl-benzenesulfonyl chloride (0.512 g, 2.008 mmol) at 0° C. The reaction mixture was stirred at room temperature for 16 h. After completion (monitored by TLC), the mixture was quenched with ice cold water and extracted with EtOAc (3×80 mL). The combined organic layer was washed with brine, dried over anhydrous Na₂SO₄, and concentrated under reduced pressure to afford title compound 32 (0.38 g, 47.97%) as a brown solid. LCMS calculated for C26H30ClN5O7S: 591.16; found: 592.63 (M+1).

### Synthesis of N-(6-((4-(aminomethyl)-1H-pyrazol-1-yl)methyl)-4-methoxybenzo[d]isoxazol-3-yl)-5-chloro-2-methoxy-4-methylbenzenesulfonamide hydrochloride (33)

To a stirred solution of compound 32 (0.38 g, 0.641 mmol) in DCM (5 mL) was added 4M HCl in Dioxane (1.6 mL, 6.418 mmol) at 0° C. The reaction mixture was stirred at room temperature for 2 h. After completion (monitored by TLC), the mixture was concentrated under reduced pressure. The crude was triturated with Et₂O (2×5 mL) to afford title compound 33 (0.30 g, HCl salt) as a brown semi solid. LCMS calculated for C21H22ClN5O5S: 491.10; found: 493.00 (M+2).

### Synthesis of N-((1-((3-((5-chloro-2-methoxy-4-methylphenyl)sulfonamido)-4-methoxybenzo[d]isoxazol-6-yl)methyl)-1H-pyrazol-4-yl)methyl)propiolamide (Syn. Ex. 21)

To a stirred solution of compound 33 (0.10 g, 0.189 mmol) in DMF (3 mL) was added DIPEA (0.13 mL, 0.756 mmol) at 0° C. The reaction mixture was stirred for 5 min followed by the addition of propiolic acid (0.015 g, 0.226 mmol) and 50% solution of T₃P in EtOAc (0.360 mL, 0.567 mmol) at 0° C. The mixture was stirred at room temperature for 1 h. After completion (monitored by TLC), the mixture was quenched with ice cold water and extracted with EtOAc (3×20 mL). The combined organic layer was dried over anhydrous Na₂SO₄ and concentrated under reduced pressure to afford the crude. The crude was purified by combi flash chromatography using with 95-100% EA/Heptane to afford the title compound N-((1-((3-((5-chloro-2-methoxy-4-methylphenyl)sulfonamido)-4-methoxybenzo[d]isoxazol-6-yl)methyl)-1H-pyrazol-4-yl)methyl)propiolamide (Syn. Ex. 21; 19 mg, 18.46%) as an off-white solid. (See Table 1 for analytical data)

### Synthesis of 5-chloro-2-methoxy-N-(4-methoxy-6-((4-(methylsulfonamidomethyl)-1H-pyrazol-1-yl)methyl)benzo[d]isoxazol-3-yl)-4-methylbenzenesulfonamide (Syn. Ex. 22)

To a stirred solution of compound 33 (0.12 g, 0.227 mmol) in DCM (5 mL) were added Et₃N (0.095 mL, 0.681 mmol) and methane sulfonyl chloride (0.031 g, 0.272 mmol) in DCM (0.5 mL) at 0° C. After completion (monitored by TLC), the mixture was quenched with ice-cold water and extracted with DCM (3×20 mL). The combined organic layer was dried over anhydrous Na₂SO₄ and concentrated under reduced pressure to afford the crude which was purified by prep. HPLC to afford the title compound 5-chloro-2-methoxy-N-(4-methoxy-6-((4-(methylsulfonamidomethyl)-1H-pyrazol-1-yl)methyl)benzo[d]isoxazol-3-yl)-4-methylbenzenesulfonamide (Syn. Ex. 22; 73 mg, 56.41%) as an off-white solid. (See Table 1 for analytical data)

### Examples 23-25

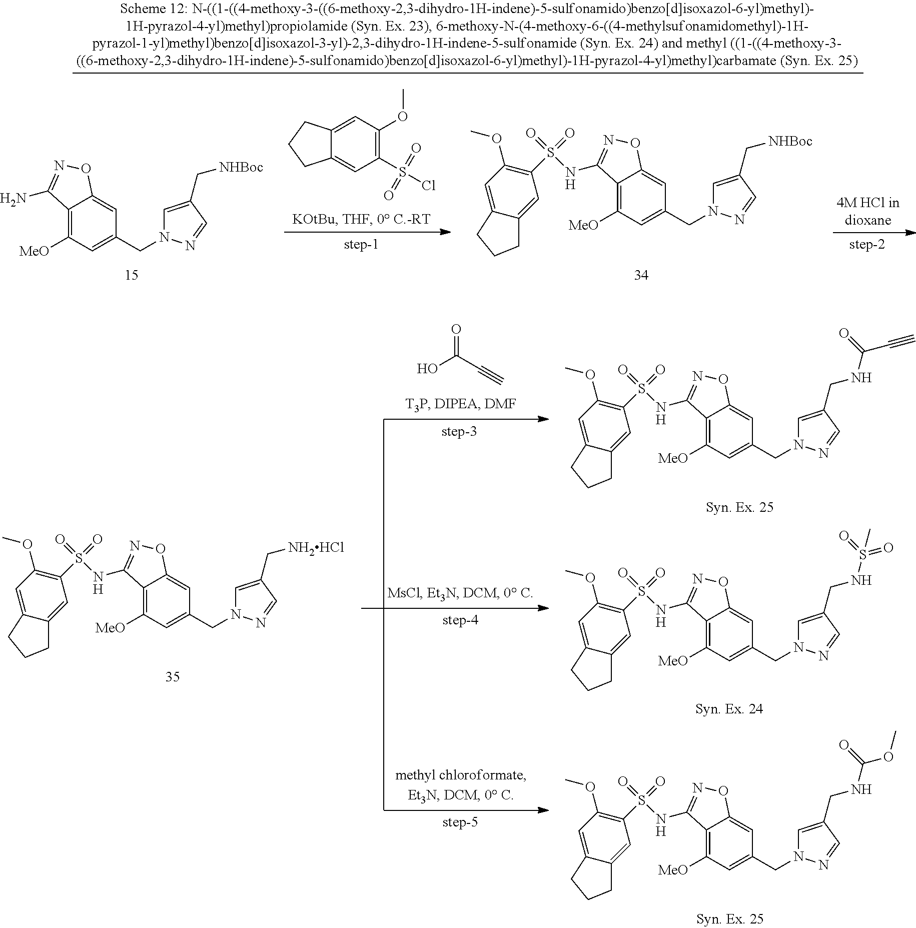

### Synthesis of tert-butyl ((1-((4-methoxy-3-((6-methoxy-2,3-dihydro-1H-indene)-5-sulfonamido) benzo[d]isoxazol-6-yl) methyl)-1H-pyrazol-4-yl) methyl) carbamate (34)

To a stirred solution of compound 15 (0.35 g, 0.937 mmol) in THF (40 mL) was added 1M solution of KOtBu in THF (2.81 mL, 2.812 mmol) and 6-methoxy-2,3-dihydro-1H-indene-5-sulfonyl chloride (0.30 g, 1.219 mmol) at 0° C. The reaction mixture was stirred at room temperature for 16 h. After completion (monitored by TLC), the mixture was quenched with water and extracted with EtOAc. The organic layer was dried over anhydrous Na₂SO₄, filtered, and concentrated under reduced pressure. The crude was purified by combi flash column chromatography using 70-75% EA:Heptane to afford title compound 34 (0.20 g, 36.55%) as an off-white solid. LCMS calculated for C28H33N5O7S: 583.21; found: 584.49 (M+1).

### Synthesis of N-(6-((4-(amino methyl)-1H-pyrazol-1-yl) methyl)-4-methoxybenzo[d]isoxazol-3-yl)-6-methoxy-2,3-dihydro-1H-indene-5-sulfonamide hydrochloride (35)

To a solution of compound 34 (0.20 g, 0.342 mmol) in DCM (2 mL) was added 4M HCl in 1,4 dioxane (2 mL) at 0° C. The reaction mixture was stirred at room temperature for 2 h. After completion (monitored by TLC), the mixture was concentrated under reduced pressure. The residue was washed with Et₂O and dried under reduced pressure to afford title compound 35 (0.16 g, 89.88%) as an off-white solid. LCMS calculated for C₂₃H25N5O5S: 483.16; found: 484.76 (M+1).

### Synthesis of N-((1-((4-methoxy-3-((6-methoxy-2,3-dihydro-1H-indene)-5-sulfonamido)benzo[d]isoxazol-6-yl)methyl)-1H-pyrazol-4-yl)methyl)propiolamide (Syn. Ex. 23)

To a stirred solution of compound 35 (0.13 g, 0.250 mmol) in DMF (5 mL) was added DIPEA (0.18 mL, 1.075 mmol) at 0° C. The reaction mixture was stirred for 5 min followed by addition of propiolic acid (0.026 g, 0.375 mmol) and 2,4,6-tripropyl-1,3,5,2,4,6-trioxatriphosphorinane-2,4,6-trioxide (0.23 g, 0.750 mmol) at 0° C. The reaction mixture was stirred at room temperature for 1 h. After completion (monitored by TLC), the mixture was quenched with ice cold water and extracted with EtOAc (3×20 mL). The combined organic layer was dried over anhydrous Na₂SO₄ and concentrated under reduced pressure. The crude was purified by combi flash chromatography using 95-100% EA/PE to afford title compound N-((1-((4-methoxy-3-((6-methoxy-2,3-dihydro-1H-indene)-5-sulfonamido)benzol[d]isoxazol-6-yl)methyl)-1H-pyrazol-4-yl)methyl)propiolamide (Syn. Ex. 23; 30 mg, 22.42%) as an off-white solid. (See Table 1 for analytical data)

### Synthesis of 6-methoxy-N-(4-methoxy-6-((4-(methylsulfonamidomethyl)-1H-pyrazol-1-yl)methyl)benzo[d]isoxazol-3-yl)-2,3-dihydro-1H-indene-5-sulfonamide (Syn. Ex. 24)

To a stirred solution of compound 35 (100 mg, 0.192 mmol) in DCM (3 mL) was added Et₃N (0.08 mL, 0.576 mmol) followed by methane sulfonyl chloride (0.018 mL, 0.230 mmol) in DCM (1 mL) at 0° C. After completion (monitored by TLC), the mixture was quenched with ice-cold water and extracted with DCM (3×100 mL). The combined organic layer was dried over anhydrous Na₂SO₄ and concentrated under reduced pressure. The crude was purified by prep. HPLC to afford the title compound 6-methoxy-N-(4-methoxy-6-((4-(methylsulfonamidomethyl)-1H-pyrazol-1-yl)methyl)benzo[d]isoxazol-3-yl)-2,3-dihydro-1H-indene-5-sulfonamide (Syn. Ex. 24; 21 mg, 19.44%) as an off-white solid. (See Table 1 for analytical data)

### Synthesis of methyl ((1-((4-methoxy-3-((6-methoxy-2,3-dihydro-1H-indene)-5-sulfonamido)benzo[d]isoxazol-6-yl)methyl)-1H-pyrazol-4-yl)methyl)carbamate (Syn. Ex. 25)

To a stirred solution of compound 35 (80 mg, 0.153 mmol) in DCM (3 mL) was added triethylamine (0.064 mL, 0.461 mmol) and methyl chloroformate (0.015 mL, 0.1836 mmol) in DCM (0.5 mL) at 0° C. After completion (monitored by TLC), the mixture was quenched with ice-cold water and extracted with DCM (3×20 mL). The combined organic layer was dried over anhydrous Na₂SO₄ and concentrated in vacuo to afford the crude which was purified by prep HPLC purification to get methyl ((1-((4-methoxy-3-((6-methoxy-2,3-dihydro-1H-indene)-5-sulfonamido)benzo[d]isoxazol-6-yl)methyl)-1H-pyrazol-4-yl)methyl)carbamate (Syn. Ex. 25; 16 mg, 19.20%) as an off-white solid. (See Table 1 for analytical data)

### Example 26

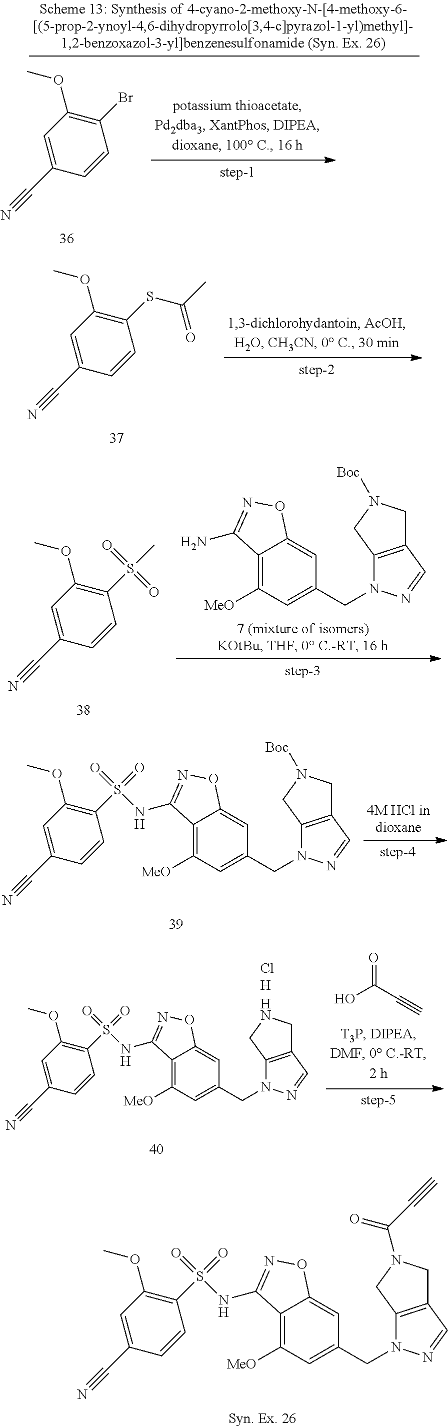

### Synthesis of S-(4-cyano-2-methoxyphenyl) ethanethioate (37)

To a solution of compound 36 (1 g, 4.715 mmol) in 1,4-Dioxane (10 mL) was added KSAc (0.646 g, 5.658 mmol), DIPEA (2.46 mL, 14.145 mmol) and followed by XantPhos (0.554 g, 0.943 mmol) at room temperature. The reaction mixture was purged with nitrogen for 10 min. followed by addition of Pd₂(dba)₃ (0.431 g, 0.47161 mmol). The mixture was stirred in sealed tube at 110° C. for 16 h. After completion (monitored by TLC and LCMS), the mixture was cooled to room temperature and filtered through a celite bed. The filtrate was diluted with H₂O (30 mL) and extracted with EtOAc (3×20 mL). The combined organic layer dried over anhydrous Na₂SO₄ and concentrated in vacuo. The crude was purified by column chromatography (neutral silica gel) using 10% EtOAc in Heptane to afford the title compound 37 (300 mg, 30.70%) as a pale-yellow solid. TLC: 20% EtOAc in Hexane (R_{f}, 0.5), LCMS Calculated for: C10H9NO2S: 207.04; Found: 208.5 (M+1).

### Synthesis of 4-cyano-2-methoxybenzenesulfonyl chloride (38)

A solution of compound 37 (0.35 g, 1.688 mmol) in ACN (14 mL) was added AcOH (0.6 mL) and water (0.35 mL) followed by 1,3-Dichloro-5,5-dimethylhydantoin (0.665 g, 3.376 mmol) at 0° C. The reaction mixture was stirred at room temperature for 1 h. After completion (monitored by TLC), the reaction mixture was quenched with ice cold water (20 mL) and extracted with EtOAc (3×20 mL). The organic layer dried over anhydrous Na₂SO₄ and concentrated in vacuo to afford the title compound 38 (650 mg, crude) as an off-white solid. TLC: 60% EtOAc in Heptane (R_{f}, 0.4). LCMS Calculated for: C8H6ClNO3S: 230.98; Found: 231.2 (M+1).

### Synthesis of tert-butyl 1-((3-((4-cyano-2-methoxyphenyl) sulfonamido)-4-methoxybenzo[d]isoxazol-6-yl) methyl)-4,6-dihydropyrrolo[3,4-c]pyrazole-5(1H)-carboxylate and tert-butyl 2-((3-((4-cyano-2-methoxyphenyl)sulfonamido)-4-methoxybenzo[d]isoxazol-6-yl)methyl)-2,6-dihydropyrrolo[3,4-c]pyrazole-5(4H)-carboxylate (39)

To a solution of compound 7 (mixture of isomers) (0.350 g, 0.908 mmol) in THF (10 mL) was added 1M solution of KOtBu in THF (2.7 mL, 2.724 mmol) at 0° C. The reaction mixture was stirred for 10 minutes followed by addition of 4-cyano-2-methoxy-benzenesulfonyl chloride 38 (0.631 g, 2.724 mmol) in THF (2 mL) at 0° C. The mixture was stirred at room temperature for 16 h. After completion (monitored by TLC and LCMS), the reaction mixture was poured into ice-cold H₂O (30 mL) and extracted with EtOAc (30 mL×3). The combined organic layer was dried over Na₂SO₄ and concentrated under reduced pressure. The crude was triturated with diethyl ether (2×10 mL) to get title compound 39 (200 mg, 37.95%) as a pale-yellow solid. TLC: 60% EtOAc in Heptane. LCMS Calculated for: C27H28N6O7S:580.17; Found: 581.2 (M+1).

### Synthesis of 4-cyano-N-(6-((5,6-dihydropyrrolo[3,4-c]pyrazol-1(4H)-yl)methyl)-4-methoxybenzo[d]isoxazol-3-yl)-2-methoxybenzenesulfonamide hydrochloride and 4-cyano-N-(6-((5,6-dihydropyrrolo[3,4-c]pyrazol-2(4H)-yl)methyl)-4-methoxybenzo[d]isoxazol-3-yl)-2-methoxybenzenesulfonamide hydrochloride (40)

To a solution of compound 39 (200 mg, 0.3445 mmol) in 1,4-Dioxane (2 mL) was added 4M HCl in 1,4 Dioxane (0.4 mL, 1.722 mmol) at 0° C. The reaction mixture was stirred at room temperature for 16 h. After completion (monitored by TLC), the mixture was concentrated in vacuo. The crude was triturated with Diethyl ether (2×10 mL) and dried in vacuo to afford the title compound 40 (180 mg, HCl salt) as a pale-yellow solid. TLC: 10% MeOH in DCM (R_{f}, 0.3). LCMS Calculated for: C₂₂H20N6O5S: 480.12 Found: 480.20 (M+1).

### Synthesis of 4-cyano-2-methoxy-N-(4-methoxy-6-((5-propioloyl-5,6-dihydropyrrolo[3,4-c]pyrazol-1(4H)-yl) methyl) benzo[d]isoxazol-3-yl) benzenesulfonamide (Syn. Ex. 26)

To a stirred solution of compound 40 (180 mg, 0.348 mmol) in DMF (2 mL) was added DIPEA (0.18 mL, 1.044 mmol), prop-2-ynoic acid (48 mg, 0.696 mmol) followed by 50% solution of T₃P in EtOAc (0.66 mL, 1.044 mmol) at 0° C. The reaction mixture was stirred at room temperature for 1 h. After completion (monitored by TLC), the mixture was quenched with H₂O (20 mL) and extracted with EtOAc (3×20 mL). The combined organic layer was dried over anhydrous Na₂SO₄ and concentrated in vacuo. The crude was purified by chiral prep-HPLC to afford the title compound 4-cyano-2-methoxy-N-[4-methoxy-6-[(5-prop-2-ynoyl-4,6-dihydropyrrolo[3,4-c]pyrazol-1-yl)methyl]-1,2-benzoxazol-3-yl]benzenesulfonamide (Syn. Ex. 26; 26 mg, 13.97%) as an off-white solid. TLC: 10% MeOH in DCM (R_{f}; 0.5). (See Table 1 for analytical data). The other regioisomer was not isolated.

### Examples 27-28

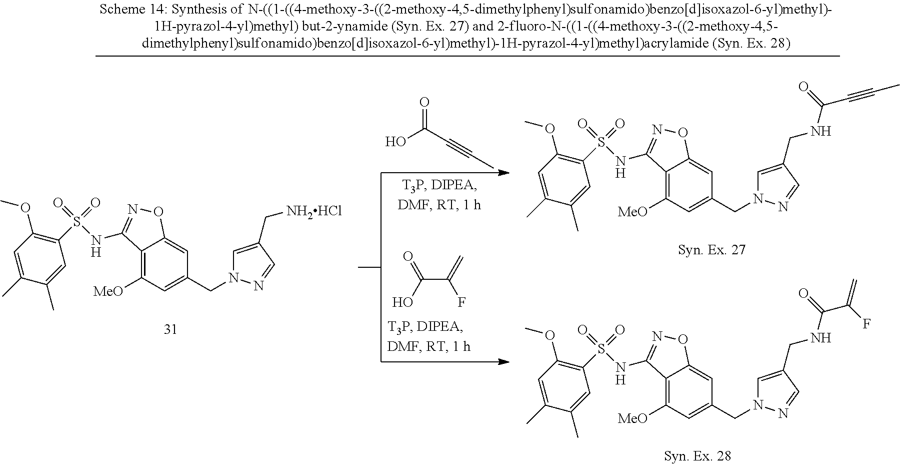

### Synthesis of N-((1-((4-methoxy-3-((2-methoxy-4,5-dimethylphenyl)sulfonamido) benzo[d]isoxazol-6-yl) methyl)-1H-pyrazol-4-yl)methyl)but-2-ynamide (Syn. Ex. 27)

To a stirred solution of compound 31 (0.11 g, 0.216 mmol) in DMF (3 mL) was added DIPEA (0.11 mL, 0.649 mmol), but-2-ynoic acid (0.036 g, 0.432 mmol) and 50% solution of T₃P in EtOAc (0.40 mL, 0.649 mmol) at 0° C. The reaction mixture was stirred at room temperature for 1 h. After completion (monitored by TLC), the mixture was diluted with water (20 mL) and extracted with EtOAc (3×20 mL). The organic layer was dried over anhydrous Na₂SO₄ and concentrated under reduced pressure. The crude obtained was purified by combi flash chromatography (neutral silica) with 80% EtOAc in Heptane to afford title compound N-((1-((4-methoxy-3-((2-methoxy-4,5-dimethylphenyl)sulfonamido)benzo[d]isoxazol-6-yl)methyl)-1H-pyrazol-4-yl)methyl)but-2-ynamide (Syn. Ex. 27; 45 mg, 38.79%) as an off-white solid. TLC: 10% MeOH in DCM. (See analytical data in Table 1).

### Synthesis of 2-fluoro-N-((1-((4-methoxy-3-((2-methoxy-4,5-dimethylphenyl) sulfonamido)benzo[d]isoxazol-6-yl)methyl)-1H-pyrazol-4-yl)methyl)acrylamide (Syn. Ex. 28)

To a stirred solution of compound 31 (0.1 g, 0.196 mmol) in DMF (3 mL) was added DIPEA (0.1 mL, 0.590 mmol), 2-fluoroprop-2-enoic acid (0.035 g, 0.392 mmol) and 50% solution of T₃P in EtOAc (0.375 mL, 0.590 mmol) at 0° C. The reaction mixture was stirred at room temperature for 1 h. After completion (monitored by TLC), the mixture was quenched with water (20 mL) and extracted with EtOAc (3×20 mL). The combined organic layer was dried over anhydrous Na₂SO₄ and concentrated under reduced pressure. The crude was purified by combi flash chromatography (neutral silica) using 80% EtOAc in Heptane to afford title compound 2-fluoro-N-((1-((4-methoxy-3-((2-methoxy-4,5-dimethylphenyl)sulfonamido) benzo[d]isoxazol-6-yl)methyl)-1H-pyrazol-4-yl)methyl)acrylamide (Syn. Ex. 28; 30 mg, 28.03%) as an off white solid. (See analytical data in Table 1).

### Examples 29-30

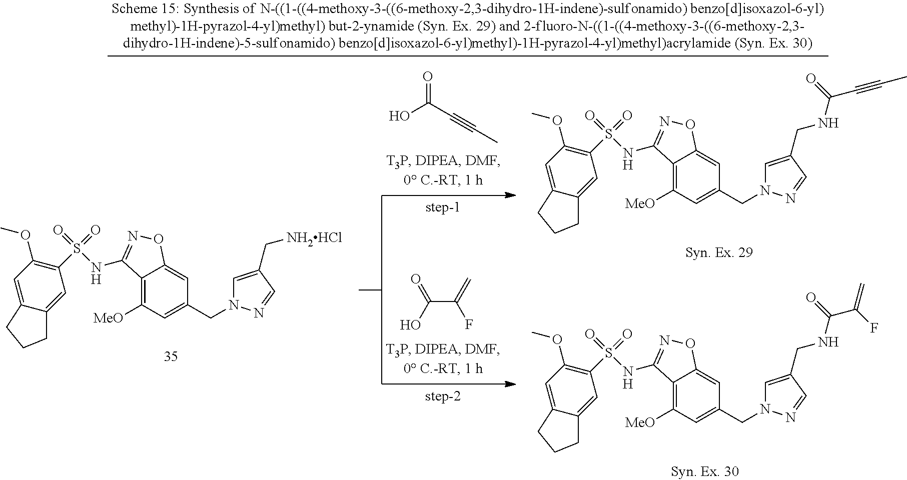

### Synthesis of N-((1-((4-methoxy-3-((6-methoxy-2,3-dihydro-1H-indene)-5-sulfonamido) benzo[d]isoxazol-6-yl) methyl)-1H-pyrazol-4-yl) methyl) but-2-ynamide (Syn. Ex. 29)

To a stirred solution of compound 35 (0.1 g, 0.192 mmol) in DMF (3 mL) was added DIPEA (0.1 mL, 0.576 mmol), but-2-ynoic acid (0.032, 0.384 mmol) and 50% solution of T₃P in EtOAc (0.366 mL, 0.576 mmol) at 0° C. The reaction mixture was stirred at room temperature for 1 h. After completion (monitored by TLC), the mixture was diluted with ice-cold water (20 mL) and extracted with EtOAc (3×20 mL). The organic layer was dried over anhydrous Na₂SO₄ and concentrated in vacuo. The crude was purified by prep HPLC to afford title compound N-((1-((4-methoxy-3-((6-methoxy-2,3-dihydro-1H-indene)-5-sulfonamido) benzo[d]isoxazol-6-yl) methyl)-1H-pyrazol-4-yl) methyl) but-2-ynamide (Syn. Ex. 29; 23 mg, 21.90%) as an off-white solid. TLC: 10% MeOH in DCM. (See analytical data in Table 1).

### Synthesis of 2-fluoro-N-((1-((4-methoxy-3-((6-methoxy-2,3-dihydro-1H-indene)-5-sulfonamido)benzo[d]isoxazol-6-yl)methyl)-1H-pyrazol-4-yl)methyl)acrylamide (Syn. Ex. 30)

To a stirred solution of compound 35 (0.10 g, 0.192 mmol) in DMF (2 mL) was added DIPEA (0.10 mL, 0.576 mmol), 2-fluoroprop-2-enoic acid (0.034 g, 0.384 mmol) and 50% solution of T₃P in EtOAc (0.366 mL, 0.576 mmol) at 0° C. The reaction mixture was stirred at room temperature for 1 h. After completion (monitored by TLC), the mixture was quenched with ice cold water (20 mL) and extracted with EtOAc (3×20 mL). The combined organic layer was dried over anhydrous Na₂SO₄ and concentrated under reduced pressure. The crude was purified by prep HPLC to afford title compound 2-fluoro-N-((1-((4-methoxy-3-((6-methoxy-2,3-dihydro-1H-indene)-5-sulfonamido)benzo[d]isoxazol-6-yl) methyl)-1H-pyrazol-4-yl)methyl)acrylamide (Syn. Ex. 30; 40 mg, 37.73%) as an off-white solid. TLC: 10% MeOH in DCM. (See analytical data in Table 1).

### Examples 31-32

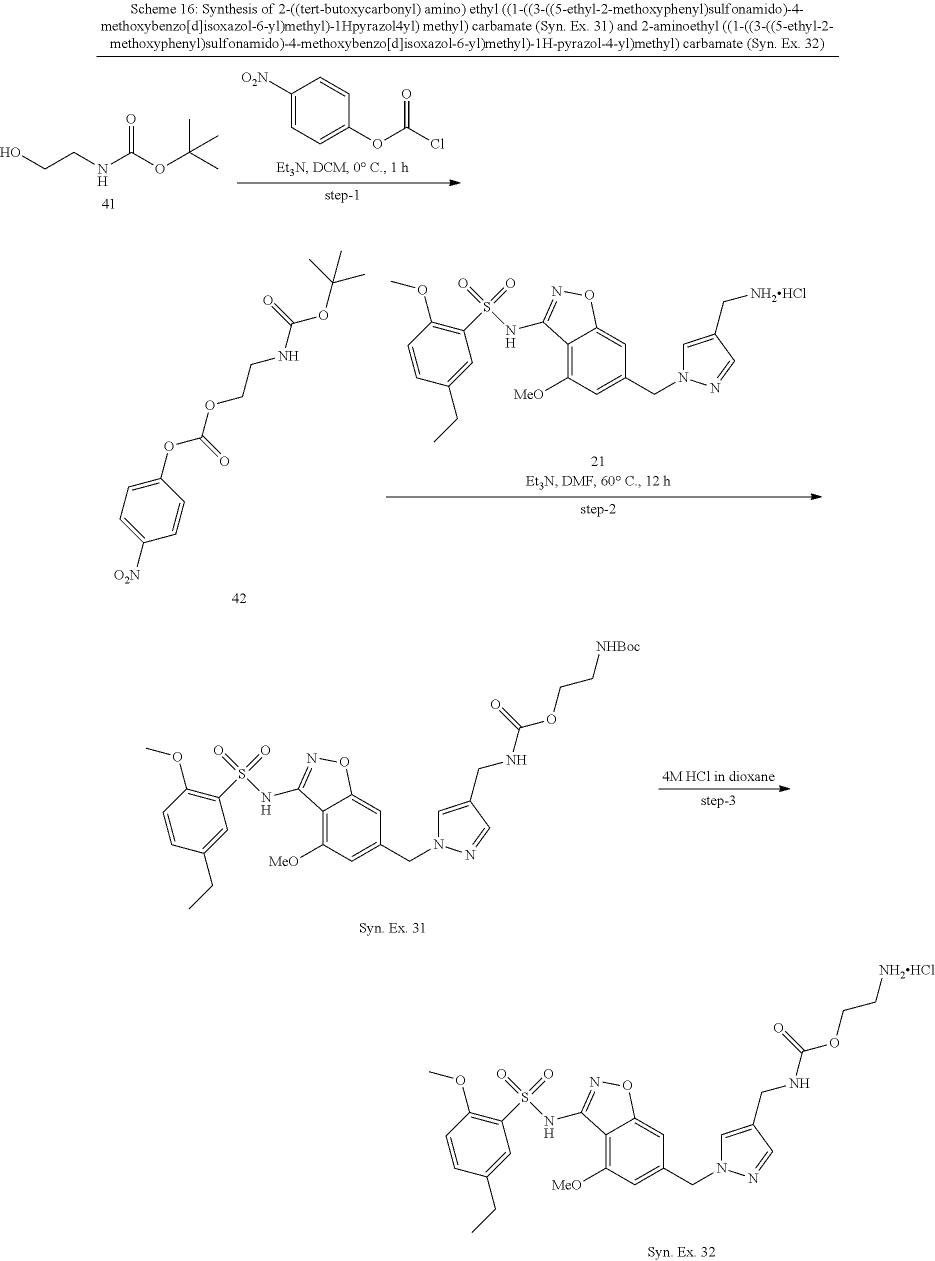

### Synthesis of tert-butyl (2-(((4-nitrophenoxy) carbonyl) oxy) ethyl) carbamate (42)

To a stirred solution of compound 41 (2.0 g, 12.406 mmol) in DCM (20 mL) was added Et₃N (3.48 mL, 24.813 mmol) and 4-nitrophenyl carbonochloridate (3.75 g, 18.609 mmol) at 0° C. The reaction mixture was stirred at 80° C. for 24 h. After completion (monitored by TLC), the mixture was quenched with water and extracted with EtOAc. The organic layer was dried over anhydrous Na₂SO₄, filtered and concentrated in vacuo. The crude was purified by combi flash with 20-25% ethyl acetate in heptane to afford title compound 42 (1.50 g, 28.30%) as a pale-yellow semi-solid. 1H NMR (400 MHz, DMSO-d6) δ 8.43-8.26 (m, 2H), 7.69-7.48 (m, 2H), 7.14-7.02 (m, 1H), 4.22 (br t, J=5.4 Hz, 2H), 3.30-3.26 (m, 2H), 1.45-1.34 (m, 9H).

### Synthesis of 2-((tert-butoxycarbonyl)amino)ethyl ((1-((3-((5-ethyl-2-methoxyphenyl)sulfonamido)-4-methoxybenzo[d]isoxazol-6-yl)methyl)-1H-pyrazol-4-yl)methyl)carbamate (Syn. Ex. 31)

To a stirred solution of compound 21 (0.25 g, 0.492 mmol) in DMF (3 mL) was added Et₃N (0.2 mL, 1.476 mmol) followed by compound 42 (0.24 g, 0.738 mmol) in DMF (2 mL) drop wise at 0° C. The reaction mixture was stirred at room temperature for 3 h. After completion (monitored by TLC), the mixture was quenched with water and extracted with DCM. The organic layer was dried over anhydrous Na₂SO₄, filtered and concentrated under reduced pressure. The crude was purified by combi flash chromatography with 4% MeOH in DCM to afford title compound 2-((tert-butoxycarbonyl)amino)ethyl ((1-((3-((5-ethyl-2-methoxyphenyl)sulfonamido)-4-methoxybenzo[d]isoxazol-6-yl)methyl)-1H-pyrazol-4-yl)methyl)carbamate (Syn. Ex. 31; 0.24 g, 74.07%) as an off-white solid. (See analytical data in Table 1).

### Synthesis of 2-aminoethyl ((1-((3-((5-ethyl-2-methoxyphenyl) sulfonamido)-4-methoxybenzo[d]isoxazol-6-yl) methyl)-1H-pyrazol-4-yl) methyl) carbamate (Syn. Ex. 32)

To a stirred solution of Syn. Ex. 31 (0.13 g, 0.197 mmol) in DCM (1.5 mL) was added 4M HCl in 1,4-dioxane (1 mL) at 0° C. The reaction mixture was stirred at room temperature for 2 h. After completion (monitored by TLC), the mixture was concentrated under reduced pressure. The residue was washed with Et₂O and dried under reduced pressure to afford title compound 2-aminoethyl((1-((3-((5-ethyl-2-methoxyphenyl)sulfonamido)-4-methoxybenzo[d]isoxazol-6-yl)methyl)-1H-pyrazol-4-yl)methyl)carbamate hydrochloride (Syn. Ex. 32; 96 mg, 82.05%) as an off-white solid. (See analytical data in Table 1).

### Example 33

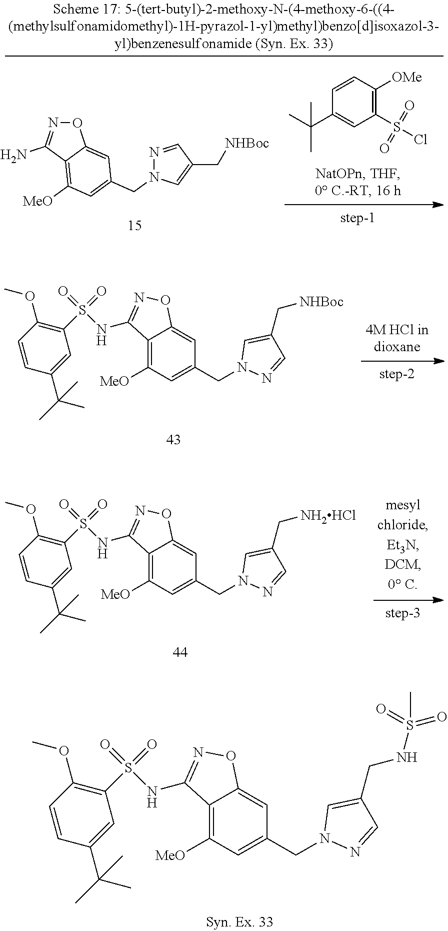

### Synthesis of tert-butyl ((1-((3-((5-(tert-butyl)-2-methoxyphenyl) sulfonamido)-4-methoxybenzo[d]isoxazol-6-yl) methyl)-1H-pyrazol-4-yl) methyl) carbamate (43)

To a solution of compound 15 (0.40 g, 1.071 mmol) in THF (15 mL) was added 1M solution of KOtBu in THF (3.2 mL, 3.213 mmol) at 0° C. The reaction mixture was stirred at 0° C. for 10 min. followed by the addition of 5-(tert-butyl)-2-methoxybenzenesulfonyl chloride (0.36 g, 1.392 mmol) in THF (2 mL). The reaction mixture was stirred at room temperature for 16 h. After completion (monitored by TLC), the mixture was quenched with water and extracted with EtOAc. The organic layer was dried over anhydrous Na₂SO₄, filtered and concentrated under reduced pressure. The crude was purified by combi flash chromatography using 0-72% EA in Heptane to afford title compound 43 (0.22 g, 34.26%) as an off-white solid. LCMS calculated for C29H37N5O7S: 599.24; Found: 600.79 (M+1).

### Synthesis of N-(6-((4-(aminomethyl)-1H-pyrazol-1-yl) methyl)-4-methoxybenzo[d]isoxazol-3-yl)-5-(tert-butyl)-2-methoxybenzenesulfonamide hydrochloride (44)

To a stirred solution of compound 43 (0.22 g, 0.366 mmol) in DCM (3 mL) was added 4M HCl in 1,4-Dioxane (2 mL) at 0° C. The reaction mixture was stirred at room temperature for 4 h. After completion (monitored by TLC), the mixture was concentrated under reduced pressure. The residue was washed with Et₂O and dried under reduced pressure to afford title compound 44 (0.17 g, 86.73%) as an off-white solid. LCMS calculated for C24H29N5O5S: 499.19; Found: 500.78 (M+1).

### Synthesis of 5-(tert-butyl)-2-methoxy-N-(4-methoxy-6-((4-(methylsulfonamidomethyl)-1H-pyrazol-1-yl)methyl)benzo[d]isoxazol-3-yl)benzenesulfonamide (Syn. Ex. 33)

To a solution of compound 44 (0.15 g, 0.279 mmol) in DCM (1 mL) was added TEA (0.11 g, 0.839 mmol) followed by methane sulfonyl chloride 5 (0.041 g, 0.362 mmol) in DCM (1 mL) drop-wise at 0° C. The reaction mixture was stirred at room temperature for 30 min. After completion (monitored by TLC), the mixture was quenched with water and extracted with DCM. The organic layer was dried over anhydrous Na₂SO₄, filtered and concentrated under reduced pressure. The crude was purified by prep HPLC to afford title compound 5-(tert-butyl)-2-methoxy-N-(4-methoxy-6-((4-(methylsulfonamidomethyl)-1H-pyrazol-1-yl)methyl)benzo[d]isoxazol-3-yl)benzenesulfonamide (Syn. Ex. 33; 85 mg, 52.79%) as an off-white solid. (See analytical data in Table 1).

### Example 34

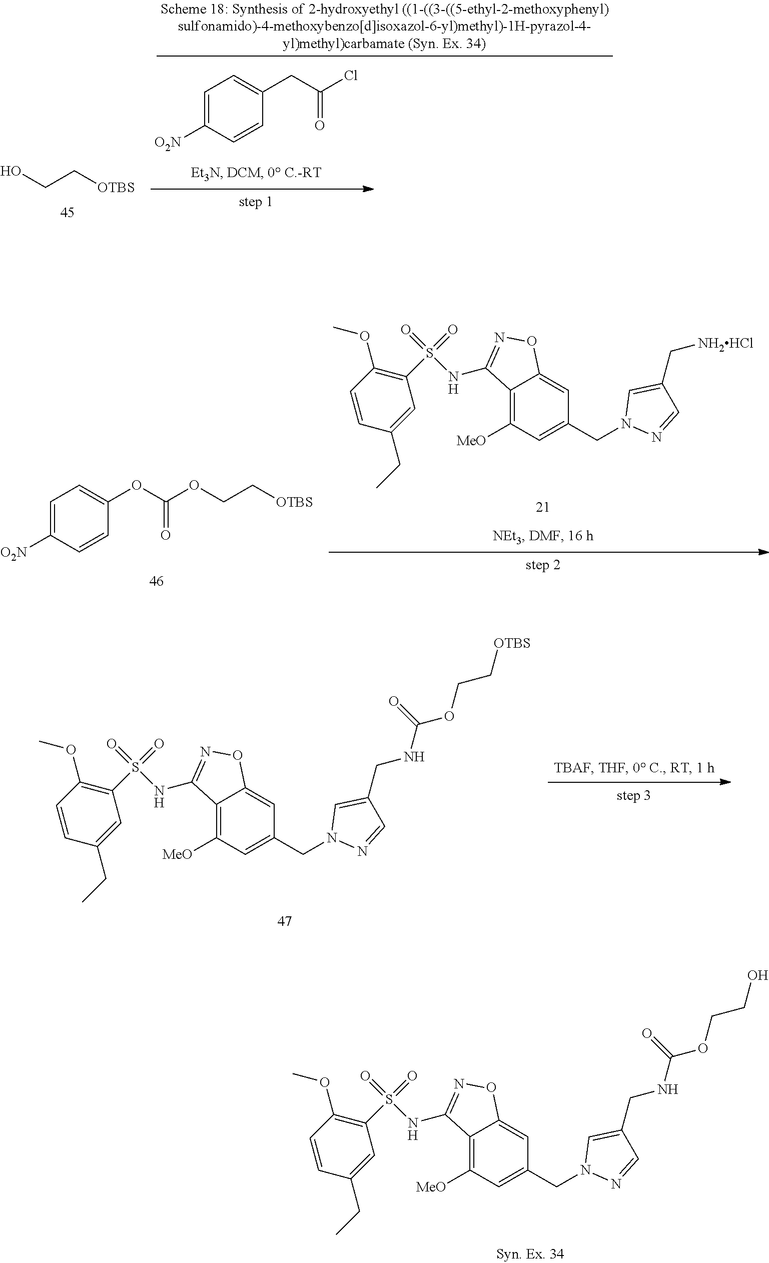

### Synthesis of 2-((tert-butyldimethylsilyl) oxy) ethyl (4-nitrophenyl) carbonate (46)

To a stirred solution of compound 45 (0.50 g, 2.835 mmol) in DCM (5 mL) was added Et₃N (1 mL, 7.087 mmol) and (4-nitrophenyl) carbonochloridate (0.56 g, 2.835 mmol) at 0° C. The reaction mixture was stirred at room temperature for 16 h. After completion (monitored by TLC), the mixture was quenched with ice-cold water (20 mL), extracted with EtOAc (3×50 mL). The organic layer was washed with brine, dried over anhydrous Na₂SO₄, filtered, and concentrated under reduced pressure. The crude was purified by combi flash chromatography using 7-12% of EA in Heptane to afford title compound 46 (0.32 g, 33.05%) as a brown oil. LCMS calculated for C15H23NO6Si: 341.13; Found: the expected product from the reaction was not ionised as evidenced from LCMS.

### Synthesis of 2-((tert-butyldimethylsilyl) oxy) ethyl ((1-((3-((5-ethyl-2-methoxyphenyl) sulfonamido)-4-methoxybenzo[d]isoxazol-6-yl) methyl)-1H-pyrazol-4-yl) methyl) carbamate (47)

To a stirred solution of compound 21 (0.25 g, 0.492 mmol) in DMF (3 mL) was added Et₃N (0.2 mL, 1.476 mmol) followed by of compound 46 (0.251 g, 0.738 mmol) at 0° C. The reaction mixture was stirred at room temperature for 4 h. After completion (monitored by TLC), the mixture was quenched with ice-cold water (50 mL), extracted with EtOAc (3×50 mL) and the combined organic layer was washed with brine, dried over anhydrous Na₂SO₄, filtered and concentrated under reduced pressure. The crude was purified by combi flash chromatography with 80-90% EA in Heptane to afford title compound 47 (0.16 g, 48.33%) as a brown solid. LCMS calculated for C31H43N5O8SSi: 673.26; Found: 674.80 (M+1).

### Synthesis of 2-hydroxyethyl ((1-((3-((5-ethyl-2-methoxyphenyl) sulfonamido)-4-methoxybenzo[d]isoxazol-6-yl)methyl)-1H-pyrazol-4-yl)methyl)carbamate (Syn. Ex. 34)

To a stirred solution of compound 47 (0.14 g, 0.207 mmol) in THF (2.0 mL) at 0° C. was added 1M TBAF solution in THF (0.4 mL, 0.4155 mmol). The reaction mixture was stirred for 1 h at room temperature. After completion (monitored by TLC), the mixture was concentrated under reduced pressure to obtain brown gummy compound which was purified by prep HPLC to afford title compound 2-hydroxyethyl ((1-((3-((5-ethyl-2-methoxyphenyl)sulfonamido)-4-methoxybenzo[d]isoxazol-6-yl)methyl)-1H-pyrazol-4-yl)methyl)carbamate (Syn. Ex. 34; 17 mg, 14.65%) as an off-white solid. (See analytical data in Table 1).

### Example 35

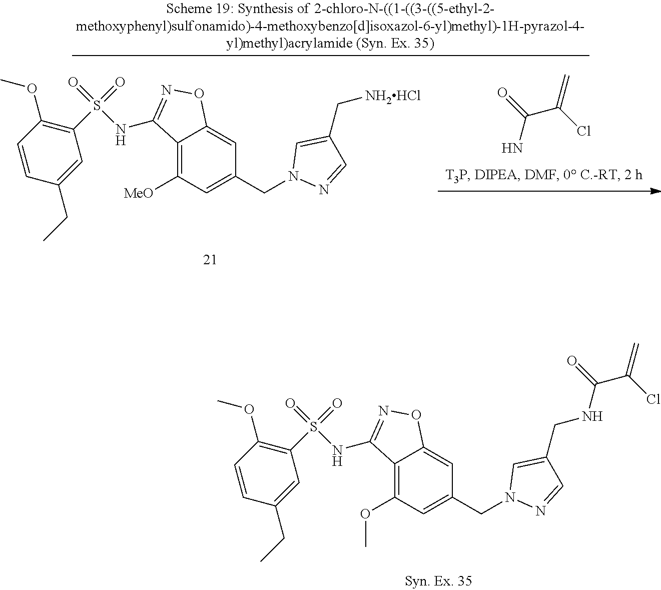

### Synthesis of 2-chloro-N-((1-((3-((5-ethyl-2-methoxyphenyl)sulfonamido)-4-methoxybenzo[d]isoxazol-6-yl)methyl)-1H-pyrazol-4-yl)methyl)acrylamide (Syn. Ex. 35)

To a stirred solution of compound 21 (0.13 g, 0.255 mmol) in DMF (3 mL) was added DIPEA (0.13 mL, 0.767 mmol) and 2-chloroacrylic acid (0.040 g, 0.382 mmol) followed by 50% solution of T₃P in EtOAc (0.48 g, 0.767 mmol) at 0° C. The reaction mixture was stirred at room temperature for 1 h. After completion (monitored by TLC), the mixture was quenched with water (20 mL) and extracted with EtOAc (3×20 mL). The combined organic layer was dried over anhydrous Na₂SO₄ and concentrated under reduced pressure. The crude was purified by combi flash chromatography (neutral silica) using 80% EtOAc in Heptane to afford title compound 2-chloro-N-((1-((3-((5-ethyl-2-methoxyphenyl)sulfonamido)-4-methoxybenzo[d]isoxazol-6-yl)methyl)-1H-pyrazol-4-yl)methyl)acrylamide (Syn. Ex. 35; 10 mg, 6.97%) as an off-white solid. (See analytical data in Table 1).

### Examples 36-37

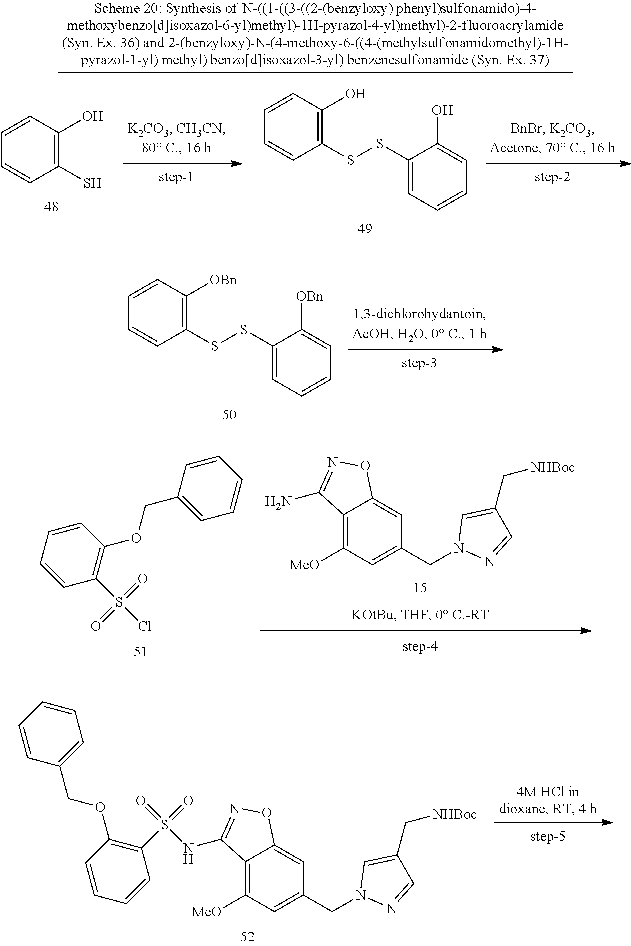 [SMILES: Oc1ccccc1S ; Oc1ccccc1SSc1ccccc1O ; c1ccc(COc2ccccc2SSc2ccccc2OCc2ccccc2)cc1 ; O=S(=O)(Cl)c1ccccc1OCc1ccccc1 ; COc1cc(Cn2cc(CNC(=O)OC(C)(C)C)cn2)cc2onc(N)c12 ; COc1cc(Cn2cc(CNC(=O)OC(C)(C)C)cn2)cc2onc(NS(=O)(=O)c3ccccc3OCc3ccccc3)c12]

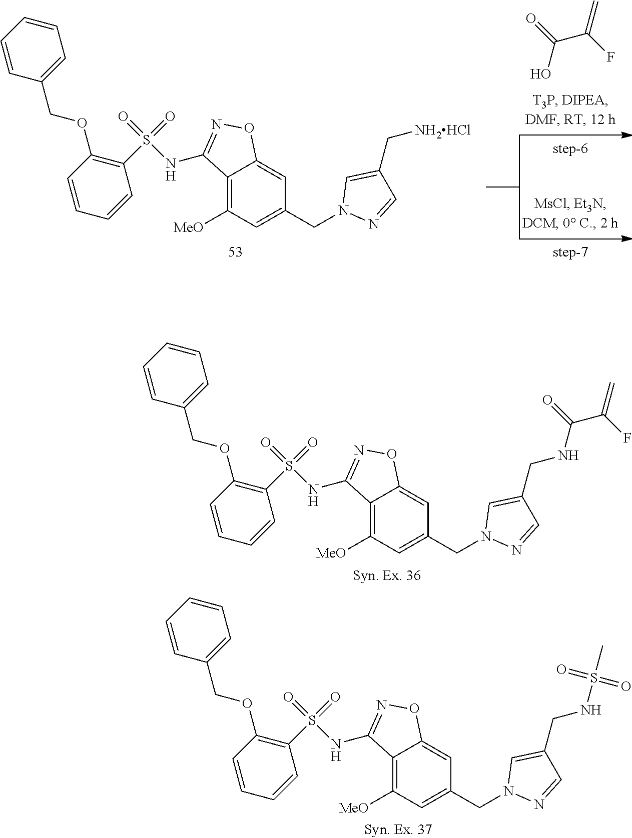

### Synthesis of 2,2′-disulfanediyldiphenol (49)

To a solution of 2-mercaptophenol 48 (4.0 g, 31.703 mmol) in ACN (60 mL) was added K₂CO₃ (13.14 g, 95.109 mmol). The reaction mixture was stirred at 80° C. for 16 h. After completion (monitored by TLC), the mixture was quenched with ice-cold water (100 mL) and extracted with EtOAc (2×50 mL). The organic layer was washed with brine (50 mL), dried over Na₂SO₄ filtered, and concentrated under reduced pressure to afford title compound 49 (3.20 g, 40.35%) as a light-yellow liquid. LCMS calculated for C12H10O2S2: 250.01; Found: 251.13 (M+1).

### Synthesis of 1,2-bis(2-(benzyloxy) phenyl) disulfane (50)

To a solution of 2,2′-disulfanediyldiphenol 49 (3.20 g, 12.783 mmol) in Acetone (32 mL) at room temperature was added K₂CO₃ (8.83 g, 63.915 mmol) and bromomethyl benzene (5.46 g, 31.957 mmol). The reaction mixture was stirred at 70° C. for 16 h. After completion (monitored by TLC), the mixture was quenched with water (100 mL) and extracted with EtOAc (2×50 mL). The organic layer was dried over anhydrous Na₂SO₄, filtered and concentrated under reduced pressure. The crude was purified by combi flash column chromatography using 5-10% EtOAc in Heptane to afford title compound 50 (3.0 g, 54.54%) as an off-white solid. LCMS calculated for C26H22O2S2: 430.11; Found: 431.20 (M+1).

### Synthesis of 2-(benzyloxy)benzenesulfonyl chloride (51)

To a solution of compound 50 (0.80 g, 1.857 mmol) in ACN (3 mL), AcOH (1.20 mL) and H₂O (0.80 mL) at 0° C. was added 1,3-dichloro-5,5-dimethylimidazolidine-2,4-dione (0.732 g, 3.716 mmol) in ACN (1.0 ml) drop wise. The reaction mixture was stirred at 0° C. for 1 h. After completion (monitored by TLC), the mixture was quenched with saturated NaHCO₃ solution (50 mL) and extracted with EtOAc (2×50 mL). The organic layer was washed with brine (50 mL), dried over Na₂SO₄, filtered, and concentrated under reduced pressure to afford title compound 51 (0.45 g, 85.71%) as an off-white solid. LCMS calculated for C13H11C103S: 282.01; Found: 263.22 [(M−1); LCMS shows sulphonic acid mass fragment].

### Synthesis of tert-butyl ((1-((3-((2-(benzyloxy)phenyl)sulfonamido)-4-methoxybenzo[d]isoxazol-6-yl)methyl)-1H-pyrazol-4-yl)methyl)carbamate (52)

To a solution of compound 15 (0.40 g, 1.071 mmol) in THF (10 mL) was added 1M solution of KOtBu in THF (3.2 mL, 3.213 mmol) at 0° C. The reaction mixture was stirred at 0° C. for 30 min. followed by addition of compound 51 (0.36 g, 1.285 mmol). The reaction mixture was stirred at room temperature for 16 h. After completion (monitored by TLC), the mixture was dried under reduced pressure. The crude was purified by combi flash chromatography (neutral silica gel) using 35-40% EtOAc in Heptane to afford title compound 52 (0.18 g, 27.14%) as a brown solid. LCMS calculated for C₃₁H33N5O7S: 619.21; Found: 620.44 (M+1).

### Synthesis of N-(6-((4-(aminomethyl)-1H-pyrazol-1-yl)methyl)-4-methoxybenzo[d]isoxazol-3-yl)-2-(benzyloxy)benzenesulfonamide hydrochloride (53)

To a solution of compound 52 (0.18 g, 0.290 mmol) in DCM (2 mL) was added 4M HCl (in Dioxane (0.2 mL, 0.87 mmol) at 0° C. The reaction mixture was stirred at room temperature for 4 h. After completion (monitored by TLC), the mixture was concentrated under reduced pressure. The residue was washed with Et₂O (3×5 mL) followed by EtOAc (5 mL) and dried under reduced pressure to afford title compound 53 (0.18 g, HCl salt) as a light brown solid. LCMS calculated for C₂₆H25N5O5S: 519.16; Found: 520.40 (M+1).

### Synthesis of Synthesis of N-((1-((3-((2-(benzyloxy)phenyl)sulfonamido)-4-methoxybenzo[d]isoxazol-6-yl)methyl)-1H-pyrazol-4-yl)methyl)-2-fluoroacrylamide (Syn. Ex. 36)

To a stirred solution of compound 53 (0.08 g, 0.143 mmol) in DMF (1 mL) was added DIPEA (0.075 mL, 0.431 mmol), 2-fluoroacrylic acid (0.025 g, 0.287 mmol) followed by 50% solution of T₃P in EtOAc (0.27 mL, 0.429 mmol) at 0° C. The reaction mixture was stirred at room temperature for 12 h. After completion (monitored by TLC), the mixture was quenched with water (30 mL) and extracted with DCM (3×30 mL). The combined organic layer was washed with brine (30 mL), dried over anhydrous Na₂SO₄ and concentrated under reduced pressure. The crude was purified by prep. HPLC to afford title compound N-((1-((3-((2-(benzyloxy) phenyl) sulfonamido)-4-methoxybenzo[d]isoxazol-6-yl) methyl)-1H-pyrazol-4-yl) methyl)-2-fluoroacrylamide (Syn. Ex. 36; 22 mg, 25.98%) as a white solid. (See analytical data in Table 1).

### Synthesis of 2-(benzyloxy)-N-(4-methoxy-6-((4-(methylsulfonamidomethyl)-1H-pyrazol-1-yl) methyl) benzo[d]isoxazol-3-yl) benzenesulfonamide (Syn. Ex. 37)

To a solution of compound 53 (0.10 g, 0.179 mmol) in DCM (2 mL) was added Et₃N (0.075 mL, 0.539 mmol) followed by methane sulfonyl chloride (0.020 g, 0.179 mmol) in DCM (1 mL) at 0° C. The reaction mixture was stirred at 0° C. for 2 h. After completion (monitored by TLC), the mixture was quenched with ice-cold water (10 mL) and extracted with DCM. The organic layer dried over anhydrous Na₂SO₄, filtered and concentrated under reduced pressure. The crude was purified by prep. HPLC to afford title compound 2-(benzyloxy)-N-(4-methoxy-6-((4-(methylsulfonamidomethyl)-1H-pyrazol-1-yl)methyl)benzo[d]isoxazol-3-yl)benzenesulfonamide (Syn. Ex. 37; 0.019 g, 17.75%) as a white solid. (See analytical data in Table 1).

### Example 38

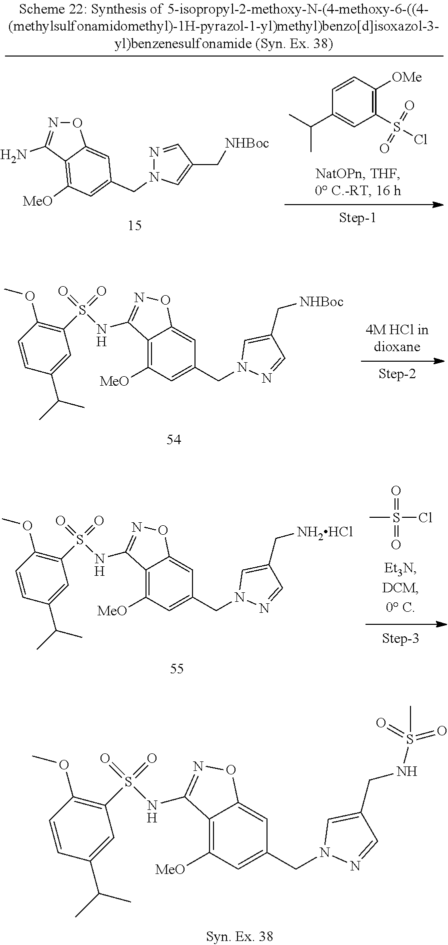

### Synthesis of tert-butyl((1-((3-((5-isopropyl-2-methoxyphenyl)sulfonamido)-4-methoxybenzo[d]isoxazol-6-yl) methyl)-1H-pyrazol-4-yl) methyl)carbamate (54)

To a solution of compound 15 (0.25 g, 0.669 mmol) in THF (10 mL) was added 1M solution of KOtBu in THF (2.0 mL, 2.008 mmol) drop wise at 0° C. The reaction mixture was stirred at 0° C. for 10 min. followed by the addition of 5-isopropyl-2-methoxybenzenesulfonyl chloride (0.21 g, 0.874 mmol) in THF (2 mL). The reaction mixture was stirred at room temperature for 16 h. After completion (monitored by TLC), the mixture was quenched with water and extracted with EtOAc. The organic layer was dried over anhydrous Na₂SO₄, filtered and concentrated under reduced pressure to afford title compound 54 (0.350 g, 89.28%) as an off-white solid. LCMS calculated for C₂₈H35N5O7S: 585.22; Found: 584.40 (M−1).

### Synthesis of N-(6-((4-(aminomethyl)-1H-pyrazol-1-yl)methyl)-4-methoxybenzo[d]isoxazol-3-yl)-5-isopropyl-2-methoxybenzenesulfonamide hydrochloride (55)

To a solution of compound 54 (0.25 g, 0.426 mmol) in DCM (3 mL) was added HCl (4 mol/L) in Dioxane (2 mL) at 0° C. The reaction mixture was stirred at room temperature for 2 h. After completion (monitored by TLC), the mixture was concentrated under reduced pressure. The residue was triturated with DCM (2×5 mL), co-distilled with Et₂O and dried under reduced pressure to afford title compound 55 (0.20 g, HCl salt) as an off-white solid. LCMS calculated for C23H27N5O5S: 485.17; Found: 484.40 (M−1).

### Synthesis of 5-isopropyl-2-methoxy-N-(4-methoxy-6-((4-(methylsulfonamidomethyl)-1H-pyrazol-1-yl)methyl)benzo[d]isoxazol-3-yl)benzenesulfonamide (Syn. Ex. 38)

To a solution of compound 55 (0.18 g, 0.344 mmol) in DCM (4 mL) was added Et₃N (0.096 mL, 0.688 mmol) at 0° C. The reaction mixture was stirred at 0° C. for 10 min. followed by drop wise addition of methane sulfonyl chloride (0.078 g, 0.688 mmol) in DCM (1 mL). The reaction mixture was stirred at room temperature for 1 h. After completion (monitored by TLC), the mixture was evaporated under reduced pressure. The crude was purified by prep HPLC to afford title 5-isopropyl-2-methoxy-N-(4-methoxy-6-((4-(methylsulfonamidomethyl)-1H-pyrazol-1-yl)methyl)benzo[d]isoxazol-3-yl)benzenesulfonamide (Syn. Ex. 38; 55 mg, 28.30%) (See analytical data in Table 1).

### Example 39

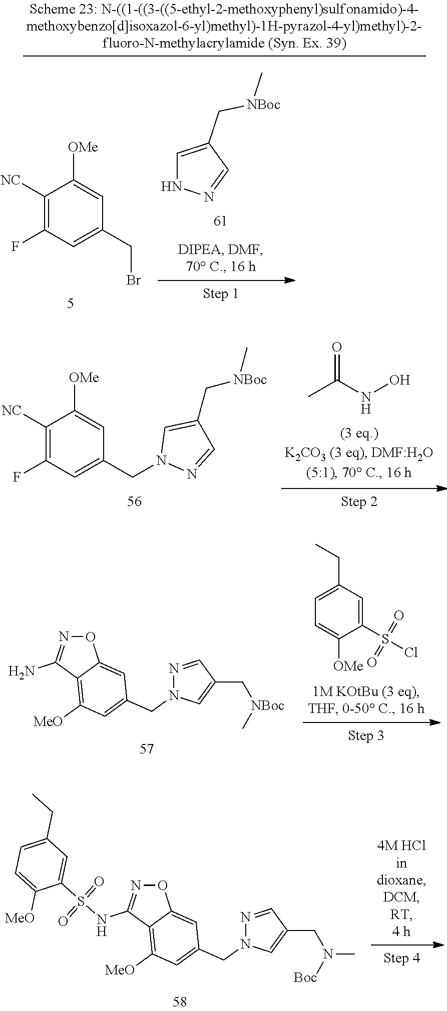

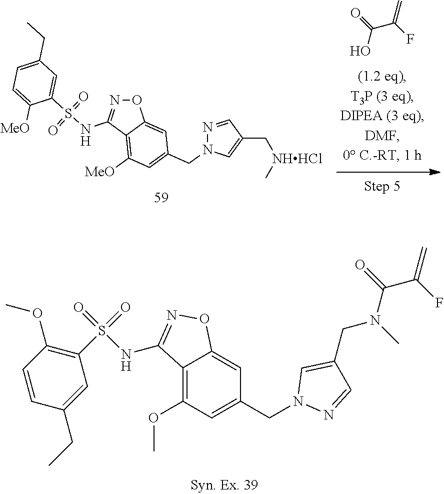

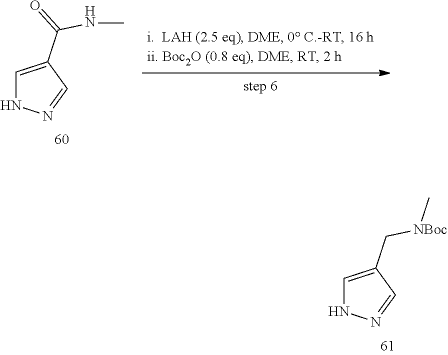

### Synthesis of tert-butyl ((1-(4-cyano-3-fluoro-5-methoxybenzyl)-1H-pyrazol-4-yl) methyl) (methyl)carbamate (56)

To a solution of compound 61 described below (0.571 g, 2.703 mmol) in DMF (6 mL) was added DIPEA (1.28 mL, 7.374 mmol) followed by compound 5 (0.60 g, 2.458 mmol). The reaction mixture was stirred at 60° C. for 6 h. After completion (monitored by TLC), the mixture was quenched with water and extracted with EtOAc. The organic layer was dried over anhydrous Na₂SO₄, filtered and concentrated under reduced pressure. The crude was purified by combi flash chromatography using 20-25% EA in Heptane to afford title compound 56 (0.55 g, 59.78%) as a pale-yellow semi solid. LCMS calculated for C19H23FN4O3: 374.17; Found: 375.59 (M+1).

### Synthesis of tert-butyl ((1-((3-amino-4-methoxybenzo[d]isoxazol-6-yl) methyl)-1H-pyrazol-4-yl)methyl)(methyl)carbamate (57)

To a solution of compound 56 (0.70 g, 1.869 mmol) in DMF (7 mL) and water (4 mL) were added K₂CO₃ (0.77 g, 5.608 mmol) and N-hydroxy acetamide (0.420 g, 5.608 mmol). The reaction mixture was stirred at 70° C. for 16 h. After completion (monitored by TLC), the mixture was diluted with water (10 mL) and extracted with EtOAc. The organic layer was dried over anhydrous Na₂SO₄, filtered and concentrated under reduced pressure to afford title compound 57 (0.65 g, 89.77%) as a yellow solid. LCMS calculated for C19H25N5O4: 387.19; Found: 388.30 (M+1).

### Synthesis of tert-butyl((1-((3-((5-ethyl-2-methoxyphenyl)sulfonamido)-4-methoxybenzo[d]isoxazol-6-yl)methyl)-1H-pyrazol-4-yl)methyl)(methyl)carbamate (58)

To a solution of compound 57 (0.60 g, 1.548 mmol) in THF (20 mL) was added 1M solution of KOtBu in THF (4.6 mL, 4.644 mmol) at 0° C. The reaction mixture was stirred at 0° C. for 10 min followed by the addition of 5-ethyl-2-methoxybenzenesulfonyl chloride (0.472 g, 2.013 mmol) in THF (2 mL). The reaction mixture was stirred at room temperature for 16 h. After completion (monitored by TLC), the mixture was quenched with water and extracted with EtOAc. The organic layer was dried over anhydrous Na₂SO₄, filtered and concentrated under reduced pressure. The crude was purified by combi flash column chromatography using 55-60% EA:Heptane to afford title compound 58 (0.55 g, 60.70%) as an off-white solid. LCMS calculated for C₂₈H35N5O7S: 585.22; Found: 586.65 (M+1).

### Synthesis of 5-ethyl-2-methoxy-N-(4-methoxy-6-((4-((methylamino)methyl)-1H-pyrazol-1-yl)methyl)benzo[d]isoxazol-3-yl)benzenesulfonamide (59)

To a solution of compound 58 (0.53 g, 0.904 mmol) in DCM (6 mL) was added 4M HCl in 1,4 Dioxane (5 mL) at 0° C. The reaction mixture was stirred at room temperature for 2 h. After completion (monitored by TLC), the mixture was concentrated under reduced pressure. The residue was washed with Et₂O and dried under reduced pressure to afford title compound 59 (0.40 g, HCl salt) as an off-white solid. LCMS calculated for C23H27N5O5S: 485.17; Found: 486.77 (M+1).

### Synthesis of N-((1-((3-((5-ethyl-2-methoxyphenyl)sulfonamido)-4-methoxybenzo[d]isoxazol-6-yl)methyl)-1H-pyrazol-4-yl)methyl)-2-fluoro-N-methylacrylamide (Syn. Ex. 39)

To a stirred solution of compound 59 (0.15 g, 0.287 mmol) in DMF (2 mL) was added DIPEA (0.15 mL, 0.862 mmol) and 2-fluoroacrylic acid (0.031 g, 0.344 mmol) and 50% T₃P in EtOAc (0.54 mL, 0.861 mmol) at 0° C. The reaction mixture was stirred at room temperature for 1 h. After completion (monitored by TLC), the mixture was quenched with water (20 mL) and extracted with EtOAc (3×20 mL). The combined organic layer was dried over anhydrous Na₂SO₄ and concentrated under reduced pressure. The crude was purified by prep. HPLC to afford title compound N-((1-((3-((5-ethyl-2-methoxyphenyl)sulfonamido)-4-methoxybenzo[d]isoxazol-6-yl)methyl)-1H-pyrazol-4-yl)methyl)-2-fluoro-N-methylacrylamide (Syn. Ex. 39; 26 mg, 16.25%) as an off-white solid. TLC: 10% MeOH in DCM. (See analytical data in Table 1).

### Synthesis of tert-butyl ((1H-pyrazol-4-yl)methyl)(methyl)carbamate (61)

To a stirred solution of compound 60 (1.0 g, 7.991 mmol) in 1,2-Dimethoxyethane (20 mL) was added LAH (2.0 mol/L) in THF (9.1 mL, 19.97 mmol) at 0° C. The reaction mixture was stirred at room temperature for 16 h. After completion (monitored by TLC), the mixture was quenched with 20% NaOH in water (80 mL) and extracted with DME. The organic layer was dried over anhydrous Na₂SO₄ and filtered. The filtrate was dissolved in 1,2-dimethoxyethane (50 mL) and added di-tert-butyl dicarbonate (1.36 mL, 6.297 mmol) at 0° C. The reaction mixture was stirred at room temperature for 2 h. After completion (monitored by TLC), the reaction mixture was quenched with water and extracted with EtOAc. The organic layer was dried over anhydrous Na₂SO₄, filtered and concentrated under reduced pressure. The crude was purified by combi flash chromatography to afford title compound 61 (0.50 g, 29.76%) as a pale-yellow liquid. LCMS calculated for C10H17N3O2: 211.13; Found: 212.30 (M+1).

### Example 40

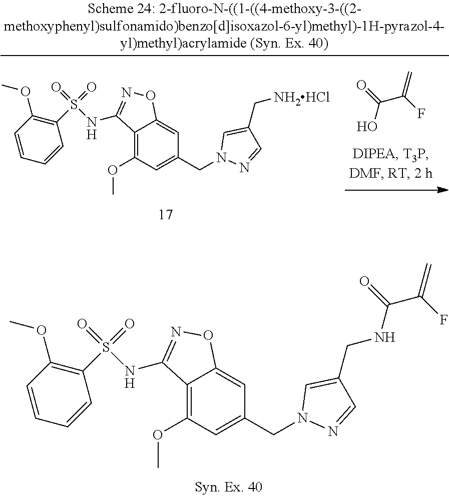

### Synthesis of 2-fluoro-N-((1-((4-methoxy-3-((2-methoxyphenyl)sulfonamido)benzo[d]isoxazol-6-yl)methyl)-1H-pyrazol-4-yl)methyl)acrylamide (Syn. Ex. 40)

To a stirred solution of compound 17 (0.14 g, 0.291 mmol) in DMF (2 mL) was added 2-fluoroacrylic acid (0.052 g, 0.582 mmol), DIPEA (0.152 mL, 0.873 mmol) at 0° C. The mixture was stirred at 0° C. for 10 min. followed by addition of 50% T₃P in EtOAc (0.55 mL, 0.873 mmol). The mixture was stirred at room temperature for 2 h. After completion (monitored by TLC), it was quenched with ice-cold water (20 mL) and extracted with EtOAc (2×100 mL). The organic layer was dried over anhydrous Na₂SO₄ and concentrated under reduced pressure. The crude was purified by prep. HPLC to afford title compound 2-fluoro-N-((1-((4-methoxy-3-((2-methoxyphenyl)sulfonamido)benzo[d]isoxazol-6-yl)methyl)-1H-pyrazol-4-yl)methyl)acrylamide (Syn. Ex. 40; 21 mg, 14.00%) as an off-white solid. (See analytical data in Table 1).

### Example 41

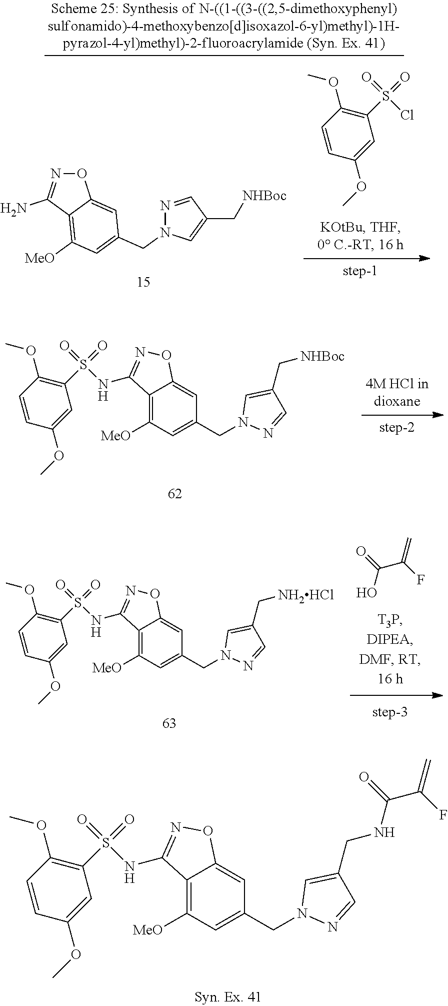

### Synthesis of tert-butyl ((1-((3-((2,5-dimethoxyphenyl)sulfonamido)-4-methoxybenzo[d]isoxazol-6-yl)methyl)-1H-pyrazol-4-yl)methyl)carbamate (62)

To a stirred solution of compound 15 (0.40 g, 1.071 mmol) in THF (8 mL) were added 1M KO^{t}Bu in THF (3.2 mL, 3.213 mmol), 2,5-dimethoxybenzenesulfonyl chloride 2 (0.506 g, 2.142 mmol) dissolved in THF (2 mL) at 0° C. The reaction mixture was stirred at room temperature for 16 h. After completion (monitored by TLC), the mixture was quenched with water and extracted with EtOAc. The organic layer was dried over anhydrous Na₂SO₄, filtered and concentrated under reduced pressure. The crude was purified by combi flash column chromatography using 65% EtOAc/Hexane to afford title compound tert-butyl((1-((3-((2,5-dimethoxyphenyl) sulfonamido)-4-methoxybenzo[d]isoxazol-6-yl) methyl)-1H-pyrazol-4-yl) methyl) carbamate 62 (0.30 g, 48.85%) as a white solid. LCMS calculated for C26H31N5O8S: 573.19; Found: 574.64 (M+1).

### Synthesis of N-(6-((4-(aminomethyl)-1H-pyrazol-1-yl)methyl)-4-methoxybenzo[d]isoxazol-3-yl)-2,5-dimethoxybenzenesulfonamide hydrochloride (63)

To a solution of compound 62 (0.30 g, 0.522 mmol) in DCM (5 mL) was added 4M HCl in 1,4 Dioxane (3 mL) at 0° C. The reaction was stirred at room temperature for 2 h. After completion (monitored by TLC), the mixture was concentrated under reduced pressure. The residue was washed with DCM (2×5 mL), co-distilled with ether, dried under reduced pressure to afford title compound 63 (0.20 g, 75.18%, HCl salt) as an off-white solid. LCMS calculated for C21H23N5O6S: 473.14; Found: 474.65 (M+1).

### Synthesis of N-((1-((3-((2,5-dimethoxy phenyl)sulfonamido)-4-methoxy benzo[d]isoxazol-6-yl) methyl)-1H-pyrazol-4-yl) methyl)-2-fluoroacrylamide (Syn. Ex. 41)

To a solution of compound 63 (0.10 g, 0.196 mmol) in DMF (2 mL) was added DIPEA (0.076 mL, 0.588 mmol), 2-fluoroacrylic acid (0.035 g, 0.392 mmol) and 50% T₃P in EtOAc (0.373 mL, 0.588 mmol) at 0° C. The mixture was stirred at room temperature for 16 h. After completion (monitored by TLC), it was quenched with ice-cold water (20 mL) and extracted with EtOAc (2×100 mL). The organic layer was dried over anhydrous Na₂SO₄ and concentrated under reduced pressure. The crude was purified by prep. HPLC to afford title N-((1-((3-((2,5-dimethoxy phenyl)sulfonamido)-4-methoxy benzo[d]isoxazol-6-yl) methyl)-1H-pyrazol-4-yl) methyl)-2-fluoroacrylamide (Syn. Ex. 41; 7 mg, 6.60%) as an off-white solid. (See analytical data in Table 1).

### Example 42

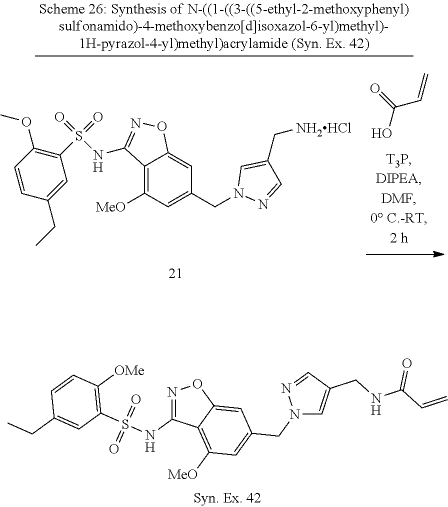

To a stirred solution of compound 21 (0.10 g, 0.196 mmol) in DMF (2 mL) was added DIPEA (0.076 mL, 0.590 mmol), acrylic acid (0.017 g, 0.235 mmol), 50% T₃P in EtOAc (0.373 mL, 0.588 mmol) at 0° C. under N₂ atmosphere. The mixture was stirred at room temperature for 2 h. After completion (monitored by TLC), the mixture was quenched with ice-cold water, and extracted with 10% MeOH/DCM (2×10 mL). The combined organic layers were dried over anhydrous Na₂SO₄ filtered and concentrated under reduced pressure. The crude was purified by prep. HPLC to afford title N-((1-((3-((5-ethyl-2-methoxyphenyl)sulfonamido)-4-methoxybenzo[d]isoxazol-6-yl) methyl)-1H-pyrazol-4-yl) methyl) acrylamide (Syn. Ex. 42; 10 mg, 9.70%) as an off-white solid. TLC: 5% MeOH in DCM; (R_{f}: 0.55), (See analytical data in Table 1).

### Examples 43-44

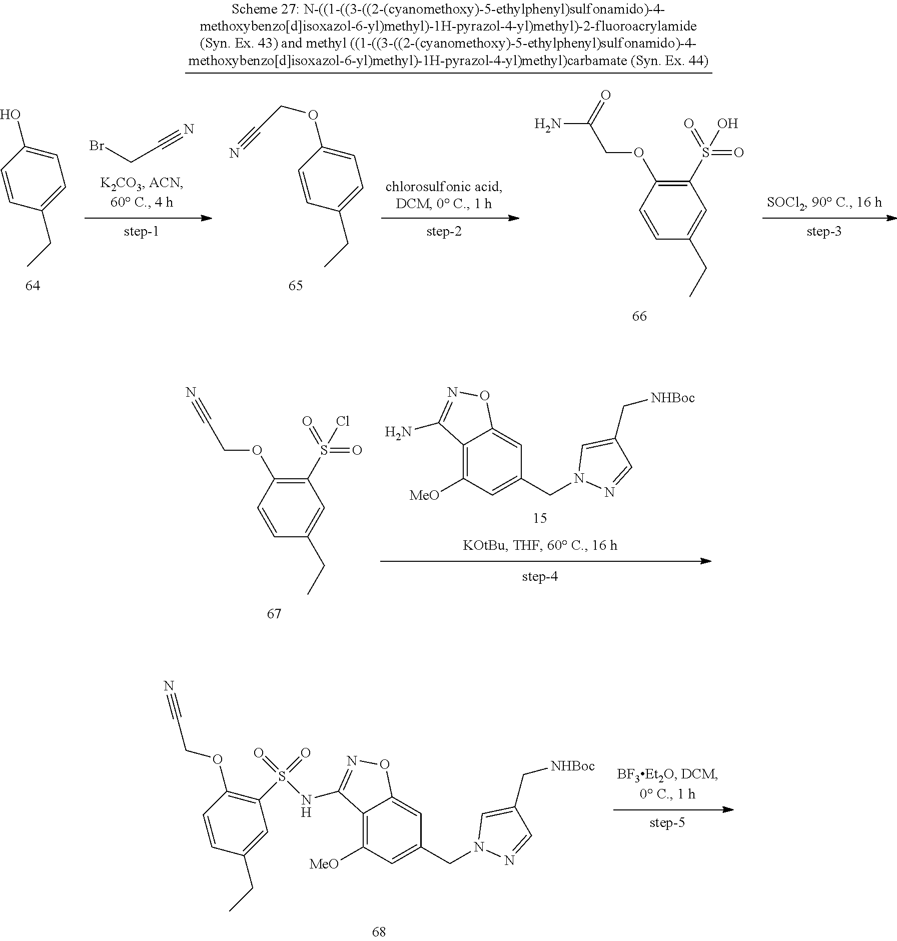

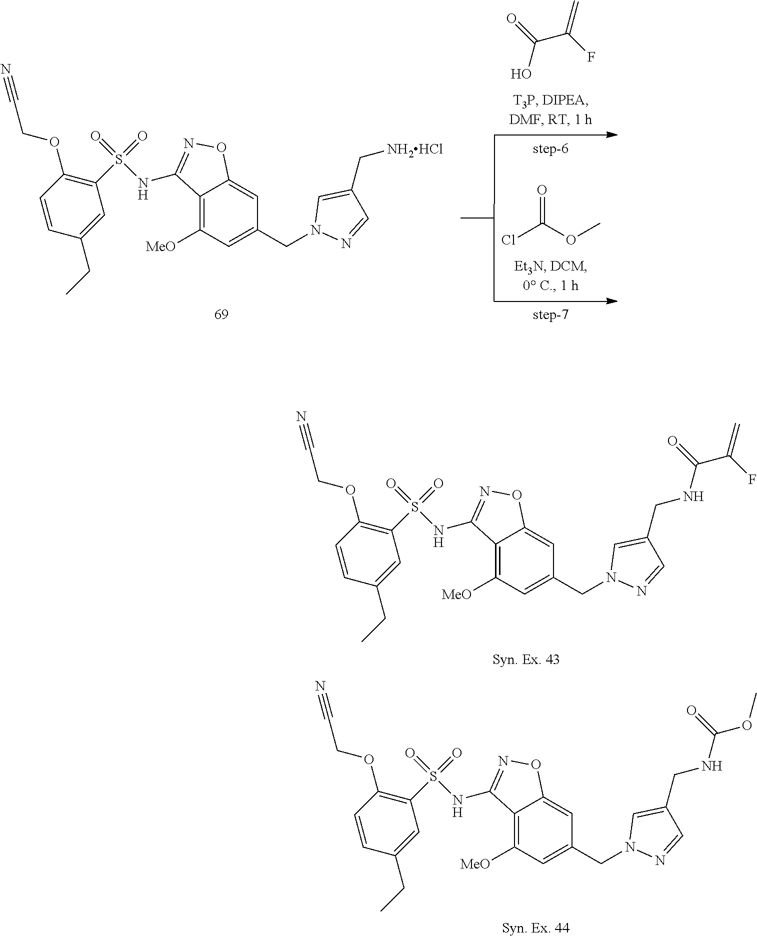

### Synthesis of 2-(4-ethylphenoxy)acetonitrile (65)

To a solution of 4-ethylphenol 64 (5.0 g, 40.926 mmol) in ACN (30 mL) were added K₂CO₃ (16.96 g, 122.77 mmol) and 2-bromoacetonitrile (7.36 g, 61.389 mmol). The mixture was stirred at 60° C. for 4 h. After completion (monitored by TLC), the mixture was quenched with water (50 mL), extracted with EtOAc (3×30 mL), dried over anhydrous Na₂SO₄ and concentrated under reduced pressure. The crude was purified by combi flash chromatography (neutral silica) with 10% EtOAc in Heptane to afford title compound 65 (6.40 g, 97.11%) as a pale-yellow liquid. TLC: 30% EtOAc in Heptane. LCMS calculated for C₁₀H11NO: 161.08; Found: 162.14 (M+1).

### Synthesis of 2-(2-amino-2-oxoethoxy)-5-ethylbenzenesulfonic acid (66)

To a solution of compound 65 (6.4 g, 39.70 mmol) in DCM (30 mL) was added chlorosulfonic acid (13.8 g, 119.10 mmol) at 0° C. The mixture was stirred at 0° C. for 1 h. After completion (monitored by TLC), the mixture was quenched with ice-cold NaHCO₃ solution (20 mL), extracted with DCM, dried over anhydrous Na₂SO₄ and concentrated under reduced pressure to afford title compound 66 (4 g, 38.87%) as an off-white solid. TLC: 30% EtOAc in Heptane (R_{f}; 0.6) LCMS calculated for C₁₀H13NO5S: 259.05; Found: 258.17 (M−1).

### Synthesis of 2-(cyanomethoxy)-5-ethylbenzenesulfonyl chloride (67)

To a compound 66 (1.5 g, 5.785 mmol) was added SOCl₂ (10 mL) at 0° C. The mixture was stirred at 90° C. for 16 h. After completion (monitored by TLC), it was concentrated under reduced pressure and co-distilled with toluene (2×10 mL). The crude was purified by column chromatography (5-10% EtOAc in Heptane over neutral silica) to afford title compound 67 (1.5 g, 99.86%) as a colorless semi-solid. TLC: 30% EtOAc in Heptane (R_{f}0.5); LCMS calculated for C₁₀H10ClNO3S: 259.01; Found: 256.99 (M−2).

### Synthesis of tert-butyl ((1-((3-((2-(cyanomethoxy)-5-ethylphenyl)sulfonamido)-4-methoxybenzo[d]isoxazol-6-yl)methyl)-1H-pyrazol-4-yl)methyl)carbamate (68)

To a stirred solution of compound 15 (0.74 g, 2.008 mmol) in THF (20 mL) was added 1M KOtBu in THF (6.0 mL, 6.024 mmol) at 0° C. The mixture was stirred for 10 min. followed by addition of compound 67 (1.04 g, 4.017 mmol) at 0° C. The mixture was stirred at 60° C. for 16 h. After completion (monitored by TLC), the mixture was quenched with ice-cold water and extracted with ethyl acetate (3×20 mL). The combined organic layer washed with brine, dried over anhydrous Na₂SO₄ and concentrated under reduced pressure. The crude was purified by combi-flash chromatography (70-80% EtOAc in heptane) with neutral silica to afford title compound 68 (0.18 g, 15.12%) as a pale-yellow solid. TLC: 80% EtOAc in Heptane. LCMS calculated for C₂₈H32N6O7S: 596.21; Found: 597.54 (M+1).

### Synthesis of N-(6-((4-(aminomethyl)-1H-pyrazol-1-yl)methyl)-4-methoxybenzo[d]isoxazol-3-yl)-2-(cyanomethoxy)-5-ethylbenzenesulfonamide (69)

To a stirred solution of compound 68 (100 mg, 0.167 mmol) in DCM (2 mL) was added 50% Boron trifluoride etherate in diethyl ether (0.056 mL, 0.200 mmol) at 0° C. The mixture was stirred at 0° C. for 1 h. After completion (monitored by TLC), the mixture was concentrated under reduced pressure. The residue was washed with Et₂O (2×10 mL) and concentrated under reduced pressure to afford title compound 69 (90 mg, HCl salt) as a light-yellow solid. TLC: 10% MeOH in DCM (R_{f}, 0.3). LCMS calculated for C23H24N6O5S: 496.15; Found: 497.71 (M+1).

### Synthesis of N-((1-((3-((2-(cyanomethoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 43)

To a stirred solution of compound 69 (90 mg, 0.168 mmol) in DMF (2 mL) were added DIEPA (0.088 mL, 0.506 mmol), 2-fluoroacrylic acid (30 mg, 0.336 mmol) and 50% T₃P in EtOAc (0.320 mL, 0.504 mmol) at 0° C. The mixture was stirred at room temperature for 1 h. After completion (monitored by TLC), the mixture was quenched with ice-cold water (20 mL) and extracted with ethyl acetate (3×20 mL). The organic layer was dried over anhydrous Na₂SO₄ and concentrated under reduced pressure. The crude was purified by prep. HPLC to afford title compound N-((1-((3-((2-(cyanomethoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 43, 10 mg, 10.41%) as an off-white solid. TLC: 10% MeOH in DCM; (R_{f}, 0.6), (See analytical data in Table 1).

### Synthesis of methyl ((1-((3-((2-(cyanomethoxy)-5-ethylphenyl)sulfonamido)-4-methoxybenzo[d]isoxazol-6-yl)methyl)-1H-pyrazol-4-yl)methyl)carbamate (Syn. Ex. 44)

To a stirred solution of compound 69 (0.12 g, 0.225 mmol) in DCM (2 mL) were added Et₃N (0.094 mL, 0.675 mmol) followed by methyl chloroformate (25 mg, 0.270 mmol) in DCM (0.5 mL) portion-wise while maintaining the pH ˜7-8. The mixture was stirred at 0° C. for 1 h. After completion (monitored by TLC), the mixture was quenched with ice-cold water (20 mL) and extracted with DCM (3×20 mL). The organic layer was dried over anhydrous Na₂SO₄ and concentrated under reduced pressure. The crude was purified by prep. HPLC to afford title compound methyl ((1-((3-((2-(cyanomethoxy)-5-ethylphenyl)sulfonamido)-4-methoxybenzo[d]isoxazol-6-yl)methyl)-1H-pyrazol-4-yl)methyl)carbamate (Syn. Ex. 44; 110 mg, 14.51%) as an off-white solid. TLC: 10% MeOH in DCM, (R_{f}, 0.55). (See analytical data in Table 1).

### Example 45

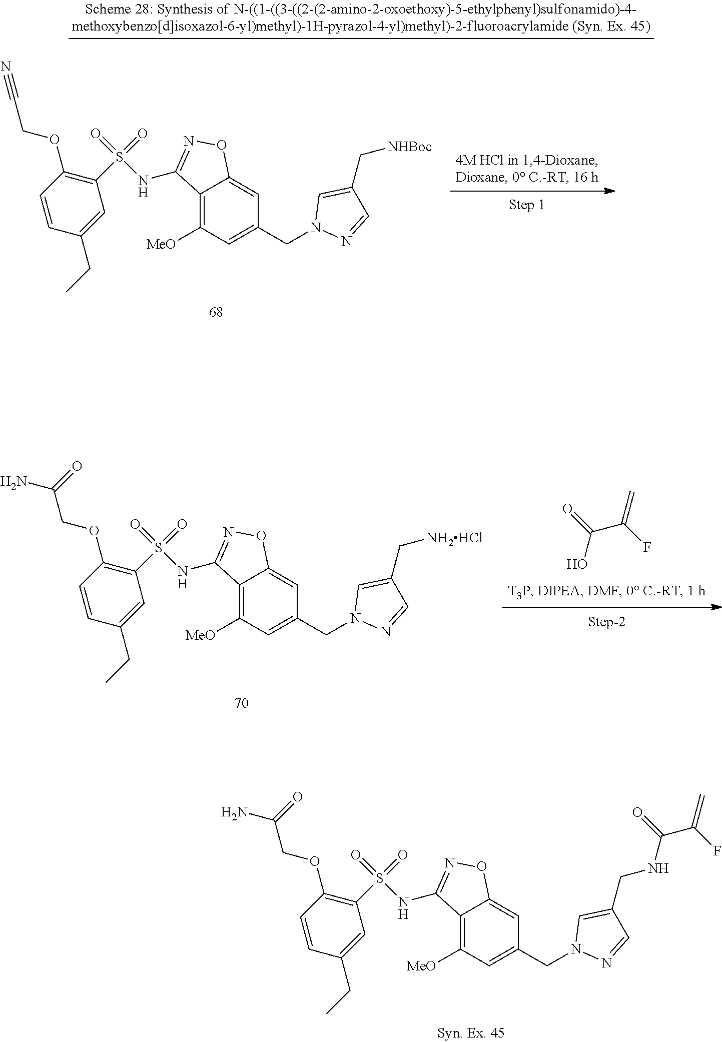

### Synthesis of 2-(2-(N-(6-((4-(aminomethyl)-1H-pyrazol-1-yl)methyl)-4-methoxybenzo[d]isoxazol-3-yl)sulfamoyl)-4-ethylphenoxy)acetamide hydrochloride (70)

To a stirred solution of compound 68 (60 mg, 0.100 mmol) in 1,4-Dioxane (1 mL) was added 4M HCl in 1,4-dioxane (0.12 mL, 0.502 mmol) at 0° C. The mixture was stirred at room temperature for 16 h. After completion (monitored by TLC), the mixture was concentrated under reduced pressure. The residue was washed with Et₂O (2×10 mL) and dried under reduced pressure to afford title compound 70 (60 mg, HCl salt) as a pale-yellow solid. TLC: 10% MeOH in DCM-(R_{f}; 0.0). LCMS calculated for C₂₃H26N6O6S: 514.16; Found: 515.39 (M+1).

### Synthesis of N-((1-((3-((2-(2-amino-2-oxoethoxy)-5-ethylphenyl) sulfonamido)-4-methoxybenzo[d]isoxazol-6-yl) methyl)-1H-pyrazol-4-yl)methyl)-2-fluoroacrylamide

To a stirred solution of compound 70 (60 mg, 0.108 mmol) in DMF (1 mL) were added DIPEA (0.057 mL, 0.326 mmol), 2-fluoroacrylic acid (19 mg, 0.216 mmol) and 50% T₃P in ethyl acetate (0.2 mL, 0.324 mmol) at 0° C. The mixture was stirred at room temperature for 1 h. After completion (monitored by TLC), the mixture was quenched with ice-cold water (10 mL) and extracted with EtOAc (3×10 mL). The organic layer was dried over anhydrous Na₂SO₄ and concentrated under reduced pressure. The crude was purified by prep. HPLC to afford title compound N-((1-((3-((2-(2-amino-2-oxoethoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 45; 10 mg, 15.87%) as an off-white solid. TLC: 5% MeOH in DCM-(R_{f}; 0.55). (See analytical data in Table 1).

### Example 46

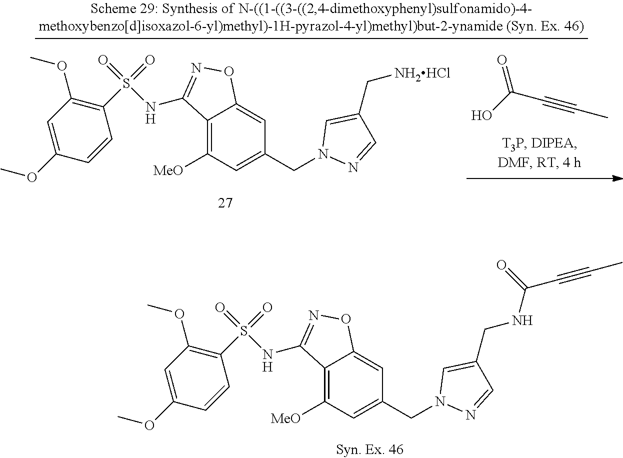

### Synthesis of N-((1-((3-((2,4-dimethoxyphenyl)sulfonamido)-4-methoxybenzo[d]isoxazol-6-yl)methyl)-1H-pyrazol-4-yl)methyl)but-2-ynamide

To a stirred solution of compound 27 (0.14 g, 0.274 mmol) in DMF (2 mL) was added DIPEA (0.14 mL, 0.823 mmol), but-2-ynoic acid (46 mg, 0.548 mmol) and 50% T₃P in ethyl acetate (0.522 mL, 0.822 mmol) at 0° C. The mixture was stirred at room temperature for 4 h. After completion (monitored by TLC), the mixture was quenched with water (50 mL), extracted with 10% MeOH in DCM (3×50 mL), dried over anhydrous Na₂SO₄, The crude was purified by prep. HPLC to afford title compound N-((1-((3-((2,4-dimethoxyphenyl)sulfonamido)-4-methoxybenzo[d]isoxazol-6-yl)methyl)-1H-pyrazol-4-yl)methyl)but-2-ynamide (Syn. Ex. 46; 15 mg, 10.13%) as an off-white solid. TLC: 10% MeOH in DCM, (R_{f}; 0.35). (See analytical data in Table 1).

### Example 47

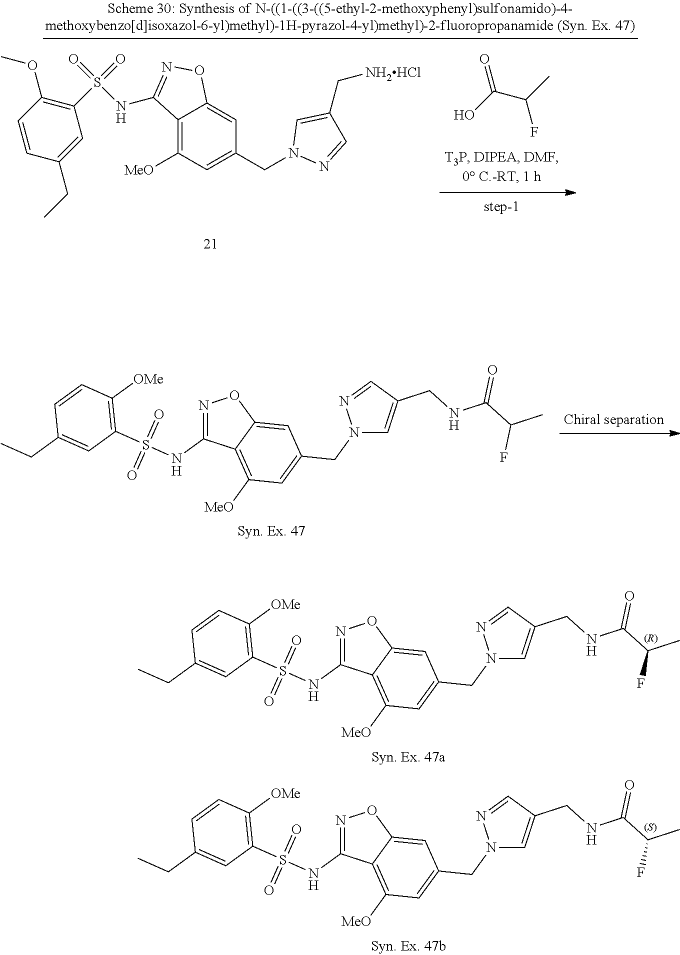

To a stirred solution of compound 21 (0.1 g, 0.196 mmol) in DMF (1 mL) was added DIPEA (0.1 mL, 0.590 mmol), 2-fluoropropanoic acid (36 mg, 0.392 mmol) and 50% T₃P in ethyl acetate (0.373 mL, 0.588 mmol) at 0° C. The mixture was stirred at room temperature for 1 h. After completion (monitored by TLC), the mixture was quenched with ice-cold water (20 mL) and extracted with ethyl acetate (2×20 mL). The organic layer was dried over anhydrous Na₂SO₄ and concentrated under reduced pressure. The crude was purified by prep. HPLC to afford title compound N-((1-((3-((5-ethyl-2-methoxyphenyl)sulfonamido)-4-methoxybenzo[d]isoxazol-6-yl)methyl)-1H-pyrazol-4-yl)methyl)-2-fluoropropanamide (Syn. Ex. 47; 57.5 mg, 53.73%) as an off-white solid. TLC: 10% MeOH in DCM-(R_{f}; 0.58). (See analytical data in Table 1). Syn. Ex. 47 (64 mg) was purified by chiral HPLC to afford (R)—N-((1-((3-((5-ethyl-2-methoxyphenyl)sulfonamido)-4-methoxybenzo[d]isoxazol-6-yl)methyl)-1H-pyrazol-4-yl)methyl)-2-fluoropropanamide (Syn. Ex. 47a; 10 mg) as an off-white solid. TLC: 10% MeOH in DCM-(R_{f}; 0.58). (See analytical data in Table 1) and (S)—N-((1-((3-((5-ethyl-2-methoxyphenyl) sulfonamido)-4-methoxybenzo[d]isoxazol-6-yl)methyl)-1H-pyrazol-4-yl)methyl)-2-fluoropropanamide (Syn. Ex. 47b; 6 mg) as an off-white solid; The stereochemistry was randomly assigned and not definitively confirmed (See analytical data in Table 1)

### Chiral Method:

Column: CHIRALPAK-IG (250×4.6 mm, 5 μm), Mobile Phase A: EtOH/MeOH, A/B: 100%, Flow: 1.0 mL/min, PDA: IG_010 IS

### Example 48

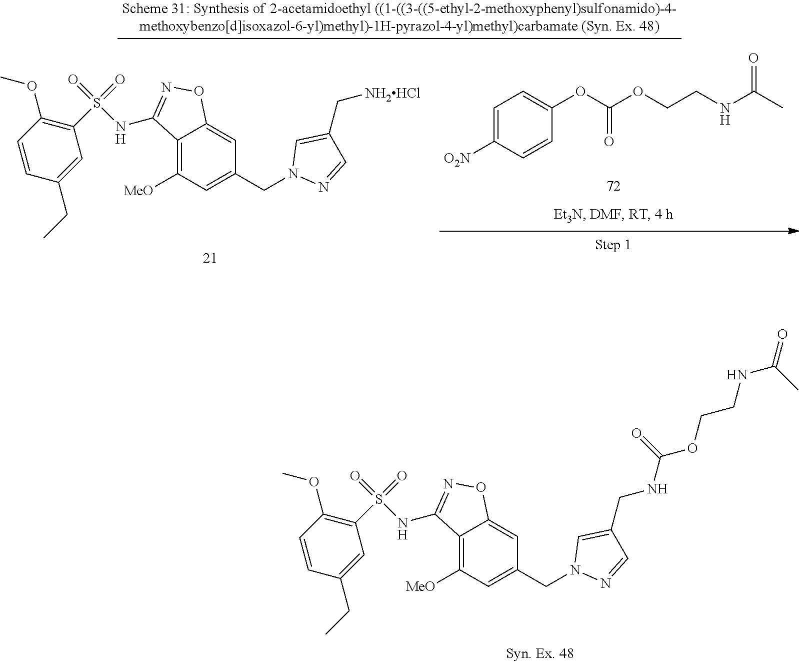

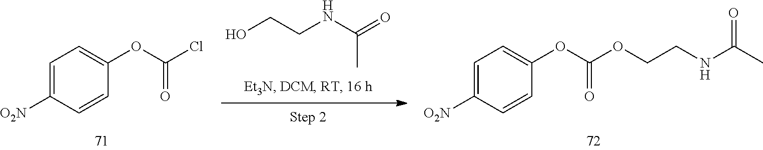

### Synthesis of 2-acetamidoethyl (4-nitrophenyl) carbonate (72)

To a stirred solution of N-(2-hydroxyethyl)acetamide (0.51 g, 4.94 mmol) in DCM (10 mL) were added Et₃N (2.10 mL, 14.82 mmol) followed by 4-nitrophenyl carbonochloridate 71 (0.995 g, 4.94 mmol) at 0° C. The mixture was stirred at room temperature for 16 h. After completion (monitored by TLC), the mixture was quenched with ice-cold water (20 mL) and extracted with EtOAc (3×50 mL). The combined organic layer was washed with brine and dried over anhydrous Na₂SO₄ and concentrated under reduced pressure to afford title compound 72 (0.60 g, 45.45%) as a yellow oil. LCMS calculated for C11H12N2O6: 268.07; Found: 269.13 (M+1).

### Synthesis of 2-acetamidoethyl ((1-((3-((5-ethyl-2-methoxyphenyl)sulfonamido)-4-methoxybenzo[d]isoxazol-6-yl)methyl)-1H-pyrazol-4-yl)methyl)carbamate (Syn. Ex. 48)

To a stirred solution of compound 21 (0.14 g, 0.275 mmol) in DMF (3 mL) was added Et₃N (0.115 mL, 0.826 mmol) and compound 72 (0.088 g, 0.330 mmol) at 0° C. The mixture was stirred at room temperature for 4 h. After completion (monitored by TLC), the mixture was quenched with ice-cold water (20 mL) and extracted with EtOAc (3×50 mL). The combined organic layer was dried over anhydrous Na₂SO₄ and concentrated under reduced pressure. The crude was purified prep. HPLC to afford title compound 2-acetamidoethyl ((1-((3-((5-ethyl-2-methoxyphenyl)sulfonamido)-4-methoxybenzo[d]isoxazol-6-yl)methyl)-1H-pyrazol-4-yl)methyl)carbamate (Syn. Ex. 48; 55 mg, 33.33%) as an off-white solid. (See analytical data in Table 1).

### Example 49

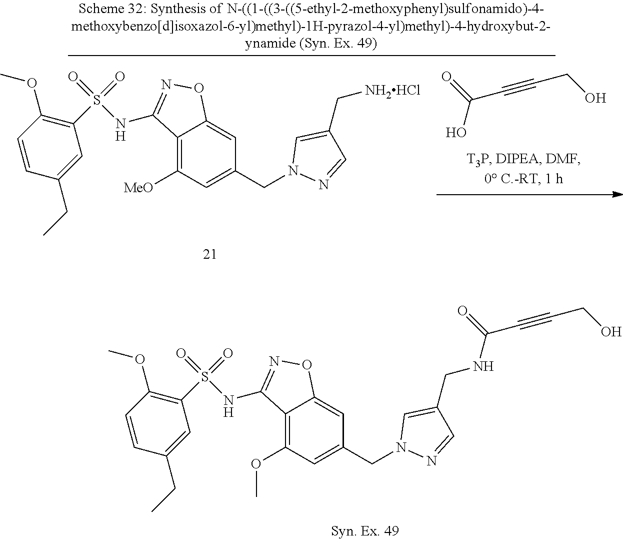

To a stirred solution of compound 21 (0.10 g, 0.196 mmol) in DMF (2 mL) were added DIPEA (0.10 mL, 0.588 mmol), 4-hydroxybut-2-ynoic acid (39 mg, 0.392 mmol) followed by 50% T₃P in ethyl acetate (0.37 mL, 0.588 mmol) at 0° C. The mixture was stirred at room temperature for 1 h. After completion (monitored by TLC), the mixture was quenched with ice-cold water (20 mL) and extracted with ethyl acetate (3×20 mL). The organic layer dried over anhydrous Na₂SO₄ and concentrated under reduced pressure. The crude was purified by prep. HPLC to afford title compound N-((1-((3-((5-ethyl-2-methoxyphenyl)sulfonamido)-4-methoxybenzo[d]isoxazol-6-yl)methyl)-1H-pyrazol-4-yl)methyl)-4-hydroxybut-2-ynamide (Syn. Ex. 49; 20 mg, 18.51%) as an off-white solid. TLC: 5% MeOH in DCM; (R_{f}. 0.6), (See analytical data in Table 1).

### Example 50

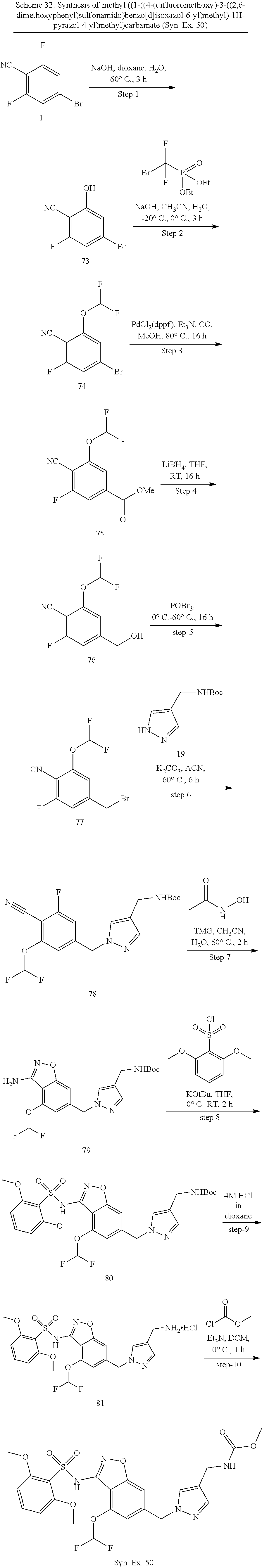

### Synthesis of 4-bromo-2-fluoro-6-hydroxybenzonitrile (73)

To a stirred solution of compound 1 (20 g, 91.743 mmol) in 1,4-Dioxane:water (400 mL, 1:1) was added NaOH (21.79 g, 544.954 mmol) portion-wise at 0° C. The mixture was stirred at 60° C. for 3 h. After completion (monitored by TLC), the mixture was diluted with water (50 mL). The aqueous layer was acidified with 1N HCl to adjust the pH˜6 and extracted with ethyl acetate (3×100 mL). The organic layer was dried over anhydrous Na₂SO₄, concentrated under reduced pressure and crude was purified by combi-flash chromatography (neutral silica) with 20% EtOAc in Heptane to afford title compound 73 (15 g, 75.71%) as an off-white solid. LCMS calculated for C₇H3BrFNO: 214.94; Found: NA. 1H NMR (400 MHz, DMSO-d6) δ 12.18 (br s, 1H), 7.55 (br d, J=6.8 Hz, 1H), 7.25 (br d, J=8.8 Hz, 1H).

### Synthesis of 4-bromo-2-(difluoromethoxy)-6-fluorobenzonitrile (74)

To a stirred solution compound 73 (15 g, 69.441 mmol) in ACN:Water (300 mL, 1:1) was added NaOH (10.8 g, 270.819 mmol) at −20° C. The mixture was stirred at −20° C. for 10 min. followed by addition of diethyl (bromodifluoromethyl)phosphonate (37.08 g, 138.882 mmol). The mixture was stirred at 0° C. and for 3 h. After completion (monitored by TLC), the reaction was diluted with ice-cold water and extracted with ethyl acetate (3×150 mL). The combined organic layer was washed with brine and dried over anhydrous Na₂SO₄, and concentrated under reduced pressure. The crude was purified by combi-flash chromatography (neutral silica) with 20% EtOAc/Heptane to afford title compound 74 (11 g, 59.55%) as a colorless liquid. LCMS calculated for C8H3BrF3NO: 264.94; Found: 266.2 (M+2).

### Synthesis of methyl 4-cyano-3-(difluoromethoxy)-5-fluorobenzoate (75)

A stirred solution of compound 74 (4 g, 15.036 mmol) in MeOH (80 mL) in a steel bomb was purged with N₂ atmosphere for 10 min. followed by addition of Et₃N (4.2 mL, 30.072 mmol) and PdCl₂(dppf) (1.8 g, 2.596 mmol). The mixture was stirred at 80° C. for 16 h under CO gas at 110 psi pressure. After completion (monitored by TLC), the mixture was filtered through a celite pad and washed with MeOH. The filtrate was concentrated under reduced pressure and crude was purified by combi-flash chromatography with 20% EtOAc/Hexane to afford title compound 75 (1.6 g, 43.47%) LCMS calculated for C10H6F3NO3: 245.03; Found: 246.5 (M+1).

### Synthesis of 2-(difluoromethoxy)-6-fluoro-4-(hydroxymethyl)benzonitrile (76)

To a stirred solution of THF (30 mL) was added 2M LiBH₄ in THF (8.97 mL, 17.947 mmol) followed by methyl 4-cyano-3-(difluoromethoxy)-5-fluorobenzoate 75 (2.20 g, 8.973 mmol) in THF (10 mL) at 0° C. The mixture was stirred at RT for 16 h. After completion (monitored by TLC), the mixture was quenched with cold water and extracted with ethyl acetate. The organic layer was washed with water, dried over anhydrous Na₂SO₄ and concentrated under reduced pressure to afford title compound 76 (1.7 g, 87.62%) as a white solid. LCMS calculated for C₉H6F3NO2: 217.04; Found: NA; 1H NMR (400 MHz, DMSO-d6) δ 7.36-7.22 (m, 2H), 5.69 (br t, J=5.1 Hz, 1H), 4.59 (br d, J=4.9 Hz, 2H).

### Synthesis of 4-(bromomethyl)-2-(difluoromethoxy)-6-fluorobenzonitrile (77)

To a stirred solution of compound 76 (1 g, 4.605 mmol) in 1,2-Dichloroethane (10 mL) was added POBr₃ (2.5 g, 9.210 mmol) at 0° C. The mixture was stirred at 60° C. for 16 h. After completion (monitored by TLC), the mixture was quenched with ice-cold water and extracted with DCM (3×100 mL). The organic layer was washed with brine, dried over anhydrous Na₂SO₄, filtered and concentrated under reduced pressure. The crude was purified by combi-flash chromatography with 12-18% EtOAc in heptane to afford title compound 77 (0.90 g, 70.31%) as a brown gummy solid. LCMS calculated for C9H5BrF3NO: 278.95; Found: NA. ¹H NMR (400 MHz, DMSO-d₆) δ 7.73-7.25 (m, 3H), 4.76 (s, 2H).

### Synthesis of tert-butyl ((1-(4-cyano-3-(difluoromethoxy)-5-fluorobenzyl)-1H-pyrazol-4-yl)methyl)carbamate (78)

To a stirred solution of compound 77 (0.40 g, 1.428 mmol) in ACN (10 mL) were added compound 19 (0.84 g, 4.284 mmol) followed by K₂CO₃ (0.39 g, 2.856 mmol) at room temperature. The mixture was stirred at 60° C. for 6 h. After completion (monitored by TLC), the mixture was concentrated under reduced pressure, quenched with ice-cold water (100 mL) and extracted with ethyl acetate (3×200 mL). The organic layer was dried over anhydrous Na₂SO₄, concentrated under reduced pressure and crude was purified by combi-flash chromatography (neutral silica) with 40% EtOAc in heptane to afford title compound 78 (0.20 g, 35.97%) as a colourless liquid. LCMS calculated for C₁₈H19F3N4O3: 396.14; Found: 397.10 (M+1).

### Synthesis of tert-butyl ((1-((3-amino-4-(difluoromethoxy) benzo[d]isoxazol-6-yl)methyl)-1H-pyrazol-4-yl)methyl)carbamate (79)

To a stirred solution of compound 78 (0.40 g, 1.009 mmol) in acetonitrile (5 mL) and water (1 mL) was added acetohydroxamic acid (0.11 g, 1.513 mmol) and 1,1,3,3-tetramethylguanidine (1.47 g, 12.765 mmol) at room temperature. The mixture was stirred at 60° C. for 2 h. After completion (monitored by TLC), the reaction was quenched with water (50 mL) and extracted with ethyl acetate (2×80 mL). The organic layer was dried over anhydrous Na₂SO₄ and concentrated under reduced pressure. The crude was purified by combi-flash chromatography with 40% EtOAc/Heptane to afford title compound 79 (0.30 g, 72.63%) as a colourless liquid. LCMS calculated for C18H21F2N5O4: 409.16; Found: 410.2 (M+1).

### Synthesis of tert-butyl ((1-((4-(difluoromethoxy)-3-((2,6-dimethoxyphenyl) sulfonamido) benzo[d]isoxazol-6-yl)methyl)-1H-pyrazol-4-yl)methyl)carbamate (80)

To a stirred solution compound 79 (180 mg, 0.4397 mmol) in dry THF (10 mL) was added 1M KO^{t}Bu in THF (1.31 mL, 1.319 mmol) at 0° C. The reaction was stirred for 10 min followed by addition of 2,6-dimethoxybenzenesulfonyl chloride (0.156 g, 0.6595 mmol) at 0° C. The mixture was stirred at room temperature for 16 h. After completion (monitored by TLC), mixture was quenched with ice cold water and acidified with 1N HCl to pH˜6.0. The aqueous layer was extracted with ethyl acetate (3×100 mL), dried over anhydrous Na₂SO₄ and concentrated under reduced pressure to get title compound 80 (100 mg crude) as a brown solid. LCMS calculated for C26H29F2N5O8S: 609.17; Found: 610.39 (M+1).

### Synthesis of N-(6-((4-(aminomethyl)-1H-pyrazol-1-yl) methyl)-4-(difluoromethoxy) benzo[d]isoxazol-3-yl)-2,6-dimethoxybenzenesulfonamide (81)

To a stirred solution of compound 80 (100 mg, 0.1640 mmol) in 1,4-Dioxane (4 mL) was added 4M HCl in 1,4 Dioxane (0.2 mL, 0.8202 mmol) at 0° C. The mixture was stirred at room temperature for 2 h. After completion (monitored by TLC), mixture was concentrated under reduced pressure. The residue was triturated with DCM (2×5 mL) was co-distilled with ether to afford title compound 81 (80 mg, HCl salt) as an off-white solid. LCMS calculated for C21H21F2N5O6S: 509.12; Found: 510.2 (M+1).

### Synthesis of methyl ((1-((4-(difluoromethoxy)-3-((2,6-dimethoxyphenyl)sulfonamido)benzo[d]isoxazol-6-yl)methyl)-1H-pyrazol-4-yl)methyl)carbamate (Syn. Ex. 50)

To a stirred solution of compound 81 (70 mg, 0.128 mmol) in DCM (10 mL) was added Et₃N (0.09 mL, 0.641 mmol) and methyl chloroformate (12 mg, 0.128 mmol) in DCM (1 mL) at 0° C. The mixture was stirred at 0° C. for 30 min. After completion (monitored by TLC), mixture was quenched with water and extracted with DCM. The organic layer was dried over anhydrous Na₂SO₄, concentrated under reduced pressure and crude was purified by prep. HPLC to afford title compound methyl ((1-((4-(difluoromethoxy)-3-((2,6-dimethoxyphenyl)sulfonamido)benzo[d]isoxazol-6-yl)methyl)-1H-pyrazol-4-yl)methyl)carbamate (Syn. Ex. 50; 4 mg, 5.55%) as an off-white solid. (See analytical Data in Table-1).

### Example 51

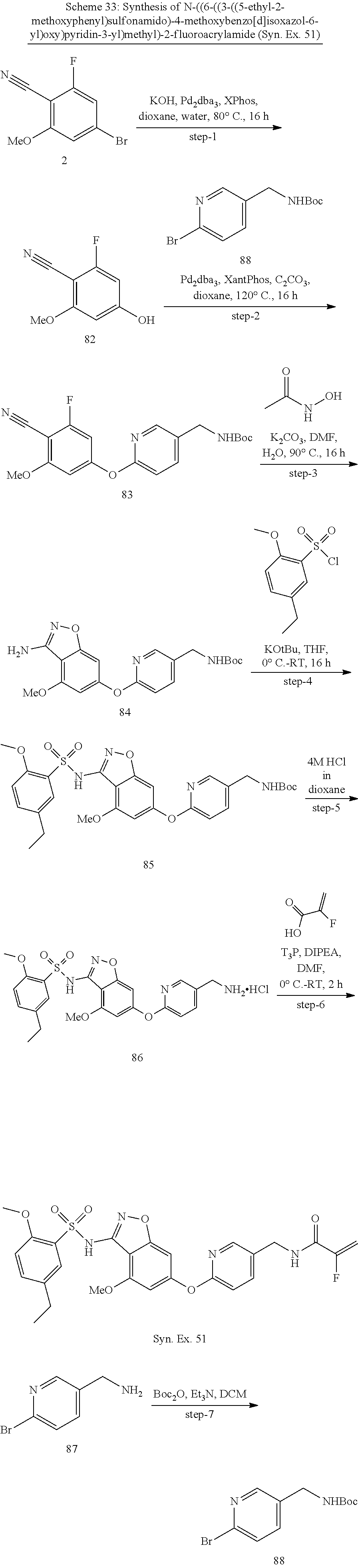

### Synthesis of 2-fluoro-4-hydroxy-6-methoxybenzonitrile (82)

To a stirred solution of compound 2 (2.80 g, 12.171 mmol) in 1,4-Dioxane (30 mL) and water (3 mL) at room temperature was added KOH (2.04 g, 36.515 mmol). The mixture was purged with nitrogen gas for 10 min followed by addition of Pd₂(dba)₃ (0.55 g, 0.608 mmol) and XPhos (0.58 g, 1.217 mmol). The mixture was stirred at 80° C. for 16 h. After completion (monitored by TLC), the mixture was filtered through a celite pad, washed with EtOAc (2×100 mL) and 10% MeOH in DCM. The filtrate was concentrated under reduced pressure and crude was triturated with Et₂O (100 mL), solid was dried under reduced pressure to afford title compound 2-fluoro-4-hydroxy-6-methoxybenzonitrile 82 (2.0 g, 98.52%) as a yellow solid. LCMS calculated for C8H6FNO2: 167.04; Found: 168.01 (M+1).

### Synthesis of tert-butyl ((6-(4-cyano-3-fluoro-5-methoxyphenoxy)pyridin-3-yl)methyl)carbamate (83)

To a stirred solution of compound 82 (1.0 g, 5.983 mmol) in 1,4-Dioxane (10 mL) was added compound 88 (1.88 g, 6.581 mmol), Cs₂CO₃ (4.87 g, 14.957 mmol) at room temperature. The mixture was degassed under N₂ atmosphere for 10 min followed by addition of Pd₂(dba)₃ (0.54 g, 0.598 mmol) and XantPhos (0.71 g, 1.196 mmol). The reaction was stirred at 120° C. for 16 h. After completion (monitored by TLC), the mixture was filtered through a celite pad and washed with EtOAc (2×200 mL). The filtrate was quenched with water and extracted with ethyl acetate. The organic layer was dried over anhydrous Na₂SO₄, concentrated under reduced pressure and crude was purified by combi-flash chromatography using 30-40% EtOAc in Heptane to afford title compound 83 (0.55 g, 24.66%) as a brown liquid. LCMS calculated for C19H20FN3O4: 373.14; Found: 374.31 (M+1).

### Synthesis of tert-butyl ((6-(4-cyano-3-fluoro-5-methoxyphenoxy)pyridin-3-yl)methyl)carbamate (84)

To a stirred solution of tert-butyl ((6-(4-cyano-3-fluoro-5-methoxyphenoxy)pyridin-3-yl)methyl)carbamate 83 (0.50 g, 1.339 mmol) in DMF (16 mL) and water (3 mL) at room temperature was added K₂CO₃ (0.92, 6.695 mmol). The mixture was stirred at room temperature for 5 min followed by addition of N-hydroxyacetamide (0.30 g, 4.017 mmol). The mixture was stirred at 90° C. for 16 h. After completion (monitored by TLC), the mixture was quenched with ice-cold water (50 mL) and the aqueous layer was extracted with EtOAc (2×100 mL). The organic layer was dried over anhydrous Na₂SO₄ and concentrated under reduced pressure. The crude was purified combi-flash chromatography using 50-60% ethyl acetate in Heptane with neutral silica to afford title compound tert-butyl ((6-(4-cyano-3-fluoro-5-methoxyphenoxy)pyridin-3-yl)methyl)carbamate 84 (0.20 g, 38.68%) as a colourless gummy syrup. LCMS calculated for C19H22N4O5: 386.16; Found: 387.77 (M+1).

### Synthesis of tert-butyl ((6-((3-((5-ethyl-2-methoxyphenyl)sulfonamido)-4-methoxybenzo[d]isoxazol-6-yl)oxy)pyridin-3-yl)methyl)carbamate (85)

To a stirred solution of tert-butyl ((6-(4-cyano-3-fluoro-5-methoxyphenoxy)pyridin-3-yl)methyl)carbamate 84 (0.15 g, 0.388 mmol) in THF (2 mL) at 0° C. was added 1M KOtBu in THF (1.16 mL, 1.164 mmol). The mixture was stirred at 0° C. under N₂ atmosphere for 10 min followed by addition of 5-ethyl-2-methoxybenzenesulfonyl chloride (0.11 g, 0.504 mmol). The mixture was stirred at room temperature for 16 h. After completion (monitored by TLC), the mixture was quenched with ice-cold water (20 mL) and extracted with ethyl acetate (2×60 mL). The organic layer was dried over anhydrous Na₂SO₄, was concentrated under reduced pressure and crude was purified by combi-flash column chromatography (60-70% of EtOAc in Heptane) over neutral silica to afford title compound 85 (0.17 g, 75.22%) as an off-white semi-solid. LCMS calculated for C28H32N4O8S: 584.19; Found: 585.30 (M+1).

### Synthesis of tert-butyl ((6-((3-((5-ethyl-2-methoxyphenyl)sulfonamido)-4-methoxybenzo[d]isoxazol-6-yl)oxy)pyridin-3-yl)methyl)carbamate (86)

To a stirred solution of compound 85 (150 mg, 0.2565 mmol) in DCM (2 mL) was added 4M HCl in 1,4 Dioxane (0.64 mL, 2.566 mmol) at 0° C. The mixture was stirred at room temperature for 3 h. After completion (monitored by TLC), the mixture was concentrated under reduced pressure. The residue was triturated with ether and dried under reduced pressure to get title compound 86 (155 mg, 0.2143 mmol, HCl salt) as an off-white gummy solid.

### Synthesis of tert-Butyl ((6-bromopyridin-3-yl)methyl)carbamate (88)

To a stirred solution of compound 87 (4 g, 21.385 mmol) in DCM (160 mL) was added Triethylamine (5.99 mL, 42.771 mmol) and (Boc)₂O (4.6 g, 21.385 mmol) drop wise. The reaction mixture was stirred at room temperature for 5 h. After completion, mixture was diluted with DCM, washed with ice-cold water and brine. The organic layer was dried over anhydrous Na₂SO₄ and concentrated under reduced pressure to get title compound 88 (6 g, 97.33%) as an off-white solid. LCMS calculated for C11H15BrN2O2: 286.03; Found: 287.13 (M+1).

### Synthesis of N-((6-((3-((5-ethyl-2-methoxyphenyl)sulfonamido)-4-methoxybenzo[d]isoxazol-6-yl)oxy)pyridin-3-yl)methyl)-2-fluoroacrylamide (Syn. Ex. 51)

To a stirred solution of compound 86 (0.15 g, 0.287 mmol) in DMF (2 mL) were added 2-fluoroacrylic acid (0.038 g, 0.431 mmol) followed by DIPEA (0.15 mL, 0.861 mmol) and T₃P 50% in EtOAc (0.365 mL, 0.574 mmol). The mixture was stirred at room temperature for 2 h. After completion (monitored by TLC), the mixture was quenched with ice-cold water and extracted with EtOAc. The organic layer was dried over anhydrous Na₂SO₄, filtered and concentrated under reduced pressure. The crude was purified by prep-HPLC purification to afford title compound N-((6-((3-((5-ethyl-2-methoxyphenyl)sulfonamido)-4-methoxybenzo[d]isoxazol-6-yl)oxy)pyridin-3-yl)methyl)-2-fluoroacrylamide (Syn. Ex. 51; 6 mg, 3.75%) as an off-white solid.

### Examples 52-53

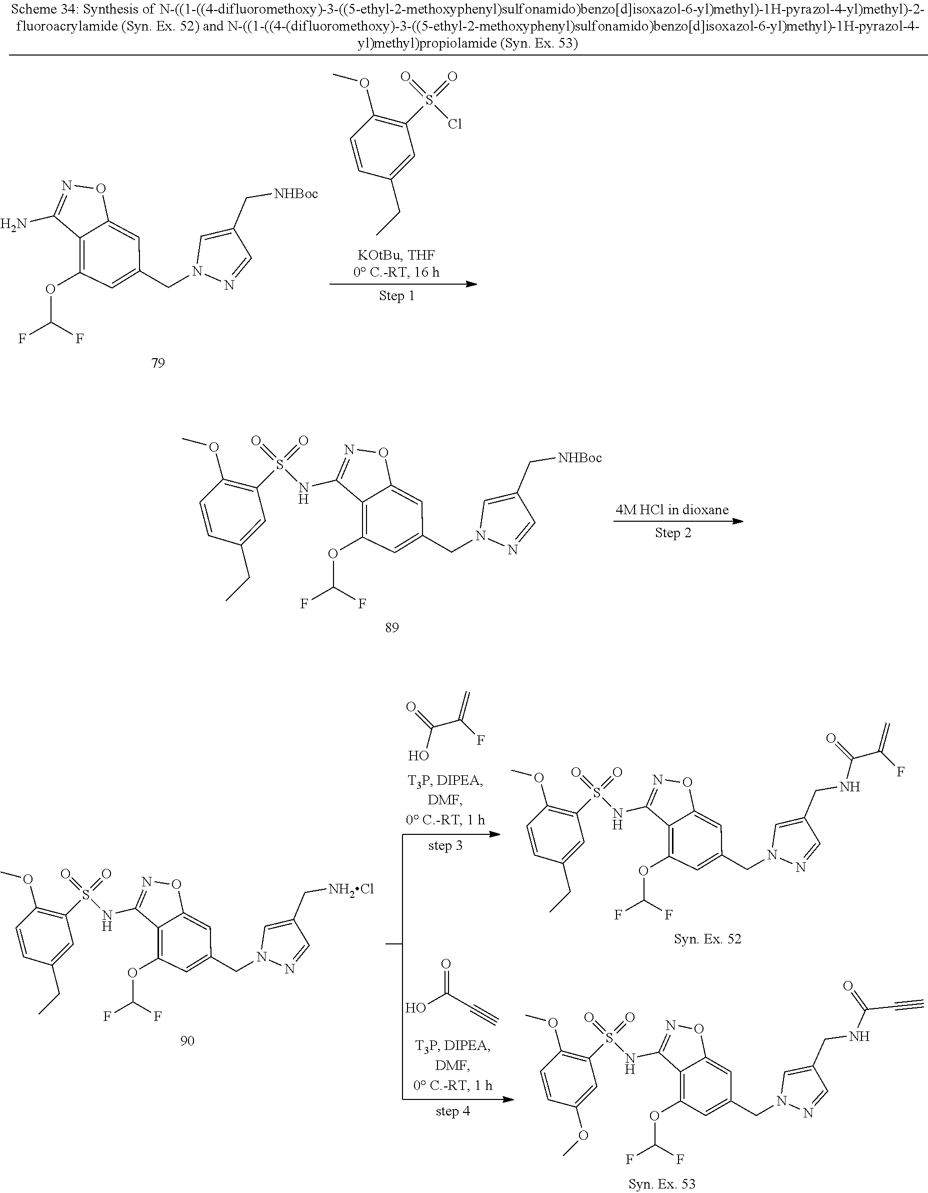

### Synthesis of tert-Butyl ((1-((4-(difluoromethoxy)-3-((5-ethyl-2-methoxyphenyl) sulfonamido) benzo[d]isoxazol-6-yl)methyl)-1H-pyrazol-4-yl)methyl)carbamate (89)

To a stirred solution of compound 79 (0.30 g, 0.732 mmol) in THF (20 mL) was added 1M KO^{t}Bu in THF (2.19 mL, 2.198 mmol) at 0° C. and stirred for 10 min. followed by addition of 5-ethyl-2-methoxybenzenesulfonyl chloride (0.257 g, 1.098 mmol). The mixture was stirred at room temperature for 16 h. After completion (monitored by TLC), the mixture was quenched with ice-cold water and extracted with EtOAc. The aqueous layer was acidified with 1N HCl to pH ˜6 and extracted with EtOAc (3×100 mL). The organic layer was washed with brine, dried over anhydrous Na₂SO₄ and concentrated under reduced pressure to afford title compound 89 (0.30 g, 67.41%) as a brown solid. LCMS calculated for C27H31F2N5O7S: 607.19; Found: 608.30 (M+1).

### Synthesis of N-(6-((4-(aminomethyl)-1H-pyrazol-1-yl) methyl)-4-(difluoromethoxy) benzo[d]isoxazol-3-yl)-5-ethyl-2-methoxybenzenesulfonamide hydrochloride (90)

To a stirred solution of compound 89 (0.30 g, 0.493 mmol) in 1,4-Dioxane (10 mL) was added 4M HCl in 1,4-Dioxane (0.085 mL, 2.465 mmol) at 0° C. The mixture was stirred at room temperature for 2 h. After completion (monitored by TLC), the mixture was concentrated under reduced pressure. The solid precipitate was washed with DCM (2×5 mL), co-distilled with Et₂O and dried under reduced pressure to afford title compound 90 (0.25 g, 93.28%) as an off-white solid. LCMS calculated for C22H23F2N5O5S: 507.14; Found: 506.2 (M−1).

### Synthesis of N-((1-((4-(difluoromethoxy)-3-((5-ethyl-2-methoxyphenyl) sulfonamido) benzo[d]isoxazol-6-yl)methyl)-1H-pyrazol-4-yl)methyl)-2-fluoroacrylamide (Syn. Ex. 52)

To a stirred solution of compound 90 (0.13 g, 0.238 mmol) in DMF (5 mL) was added DIPEA (0.125 mL, 0.716 mmol), 2-fluoroacrylic acid 4 (42 mg, 0.477 mmol) dissolved in DMF (1 mL) and 50% T₃P in EtOAc (0.45 mL, 0.716 mmol) at 0° C. The mixture was stirred at room temperature for 1 h. After completion (monitored by TLC), the mixture was quenched with ice-cold water and extracted with EtOAc. The combined organic layer was dried over anhydrous Na₂SO₄ filtered and concentrated under reduced pressure. The crude was purified by prep. HPLC to afford title compound N-((1-((4-(difluoromethoxy)-3-((5-ethyl-2-methoxyphenyl)sulfonamido)benzo[d]isoxazol-6-yl)methyl)-1H-pyrazol-4-yl)methyl)-2-fluoroacrylamide (Syn. Ex. 52; 5.0 mg, 3.62%) as an off-white solid. (See analytical data in Table 1).

### Synthesis of N-((1-((4-(difluoromethoxy)-3-((5-ethyl-2-methoxyphenyl)sulfonamido) benzo[d]isoxazol-6-yl)methyl)-1H-pyrazol-4-yl)methyl)propiolamide (Syn. Ex. 53)

To a stirred solution of compound 90 (0.13 g, 0.238 mmol) in DMF (5 mL) was added DIPEA (0.125 mL, 0.716 mmol), propiolic acid 5 (0.033 g, 0.477 mmol) dissolved in DMF (2 mL) and 50% T₃P in EtOAc (0.45 mL, 0.716 mmol) at 0° C. The mixture was stirred at room temperature for 1 h. After completion (monitored by TLC), the mixture was quenched with ice-cold water and extracted with EtOAc. The combined organic layer was dried over anhydrous Na₂SO₄ filtered and concentrated under reduced pressure. The crude was purified by prep. HPLC to afford title compound N-((1-((4-(difluoromethoxy)-3-((5-ethyl-2-methoxyphenyl)sulfonamido)benzo[d]isoxazol-6-yl)methyl)-1H-pyrazol-4-yl)methyl) propiolamide (Syn. Ex. 53; 14 mg, 10.52%) as an off-white solid. (See analytical data in Table 1).

### Examples 54-55

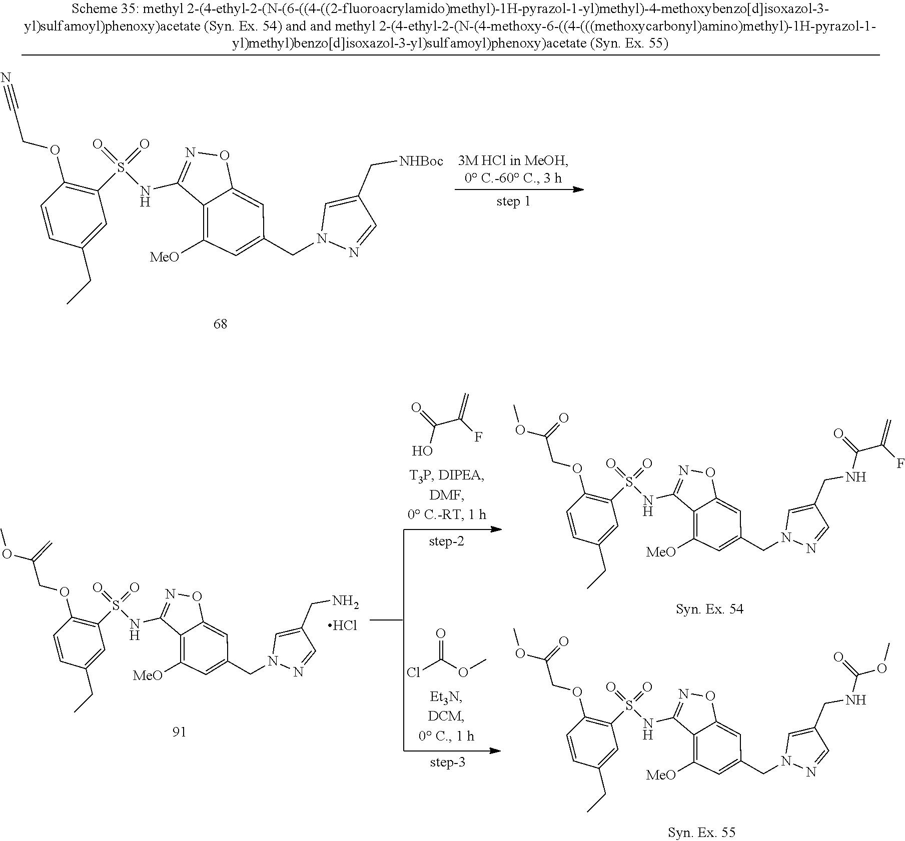

### Synthesis of methyl 2-(2-(N-(6-((4-(aminomethyl)-1H-pyrazol-1-yl)methyl)-4-methoxybenzo[d]isoxazol-3-yl)sulfamoyl)-4-ethylphenoxy)acetate hydrochloride (91)

To a stirred solution of compound 68 (0.22 g, 0.368 mmol) in MeOH (2.5 mL) was added 3M HCl in MeOH (2.45 mL, 7.360 mmol) at 0° C. The mixture was stirred at 60° C. for 3 h. After completion (monitored by TLC), the mixture was concentrated under reduced pressure and co-distilled with toluene (2×10 mL). The solid precipitate was washed with Et₂O (2×10 mL) and dried under reduced pressure to afford title compound 91 (0.20 g, HCl salt) as a light-yellow solid. LCMS calculated for C24H27N5O7S:529.16; Found: 530.93 (M+1).

### Synthesis of methyl 2-(4-ethyl-2-(N-(6-((4-((2-fluoroacrylamido)methyl)-1H-pyrazol-1-yl)methyl)-4-methoxybenzo[d]isoxazol-3-yl)sulfamoyl)phenoxy)acetate (Syn. Ex. 54)

To a stirred solution of compound 91 (0.10 g, 0.176 mmol) in DMF (2 mL) at 0° C. were added DIPEA (0.092 mL, 0.528 mmol), 2-fluoroacrylic acid 2 (31 mg, 0.352 mmol) followed by 50% T₃P in EtOAc (0.335 mL, 0.528 mmol) the mixture was stirred at room temperature for 1 h. After completion (monitored by TLC), the mixture was quenched with ice-cold water (20 mL) and extracted with EtOAc (3×20 mL). The combined organic layer was dried over anhydrous Na₂SO₄ and concentrated under reduced pressure. The crude was purified by prep. HPLC to afford title compound methyl 2-(4-ethyl-2-(N-(6-((4-((2-fluoroacrylamido)methyl)-1H-pyrazol-1-yl)methyl)-4-methoxybenzo[d]isoxazol-3-yl)sulfamoyl)phenoxy)acetate (Syn. Ex. 54; 8.0 mg, 7.54%) as an off-white solid. (See analytical data in Table 1).

### Synthesis of methyl 2-(4-ethyl-2-(N-(4-methoxy-6-((4-(((methoxycarbonyl)amino)methyl)-1H-pyrazol-1-yl)methyl)benzo[d]isoxazol-3-yl)sulfamoyl)phenoxy)acetate (Syn. Ex. 55)

To a stirred solution of compound 91 (0.10 g, 0.176 mmol) in DCM (2 mL) was added Et₃N (0.073 mL, 0.528 mmol) and methyl chloroformate (0.02 g, 0.212 mmol) in DCM (0.5 mL) at 0° C. The mixture was stirred at 0° C. for 1 h. After completion (monitored by TLC), the mixture was quenched with ice-cold water and extracted with DCM. The combined organic layer dried over anhydrous Na₂SO₄ and concentrated under reduced pressure. The crude was purified by prep. HPLC to afford title compound methyl 2-(4-ethyl-2-(N-(4-methoxy-6-((4-(((methoxycarbonyl)amino)methyl)-1H-pyrazol-1-yl)methyl)benzo[d]isoxazol-3-yl)sulfamoyl)phenoxy)acetate (Syn. Ex. 55; 11 mg, 10.67%) as an off-white solid. (See analytical data in Table 1).

### Examples 56-57

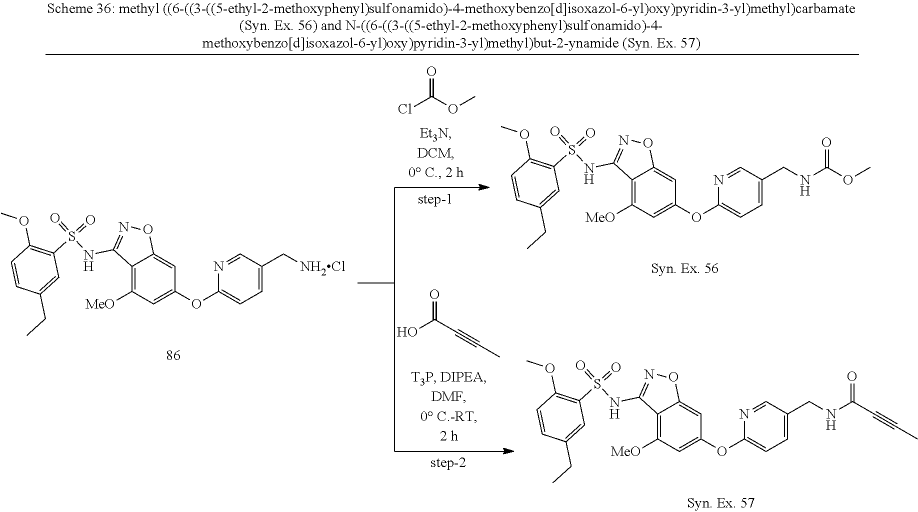

### Synthesis of methyl ((6-((3-((5-ethyl-2-methoxyphenyl)sulfonamido)-4-methoxybenzo[d]isoxazol-6-yl)oxy)pyridin-3-yl)methyl)carbamate (Syn. Ex. 56)

To a stirred solution of compound 86 (0.10 g, 0.191 mmol) in DCM (1 mL) was added Et₃N (0.053 mL, 0.382 mmol) at 0° C. The mixture was stirred for 10 min. followed by drop-wise addition of methyl chloroformate (0.018 g, 0.191 mmol) in DCM (1 mL). The mixture was stirred at 0° C. for 2 h. After completion (monitored by TLC), the mixture was quenched with ice-cold water and extracted with DCM. The combined organic layer was dried over anhydrous Na₂SO₄, filtered and concentrated under reduced pressure. The crude was purified by prep. HPLC to afford title compound methyl ((6-((3-((5-ethyl-2-methoxyphenyl) sulfonamido)-4-methoxybenzo[d]isoxazol-6-yl)oxy)pyridin-3-yl)methyl)carbamate (Syn. Ex. 56; 4 mg, 3.84%) as an off-white solid. (See analytical data in Table 1).

### Synthesis of N-((6-((3-((5-ethyl-2-methoxyphenyl)sulfonamido)-4-methoxybenzo[d]isoxazol-6-yl)oxy)pyridin-3-yl)methyl)but-2-ynamide (Syn. Ex. 57)

To a stirred solution of compound 86 (0.09 g, 0.172 mmol) in DMF (1 mL) was added DIPEA (0.09 mL, 0.516 mmol), but-2-ynoic acid (21 mg, 0.258 mmol) and 50% T3P in EtOAc (0.21 g, 0.344 mmol) at 0° C. The mixture was stirred at room temperature for 2 h. After completion (monitored by TLC), the mixture was quenched with ice-cold water and extracted with EtOAc. The combined organic layer was dried over anhydrous Na₂SO₄, filtered and concentrated under reduced pressure. The crude was purified by prep. HPLC to afford title compound N-((6-((3-((5-ethyl-2-methoxyphenyl)sulfonamido)-4-methoxybenzo[d]isoxazol-6-yl)oxy)pyridin-3-yl)methyl)but-2-ynamide (Syn. Ex. 57; 8 mg, 8.42%) as an off-white solid. (See analytical data in Table 1).

### Example 58

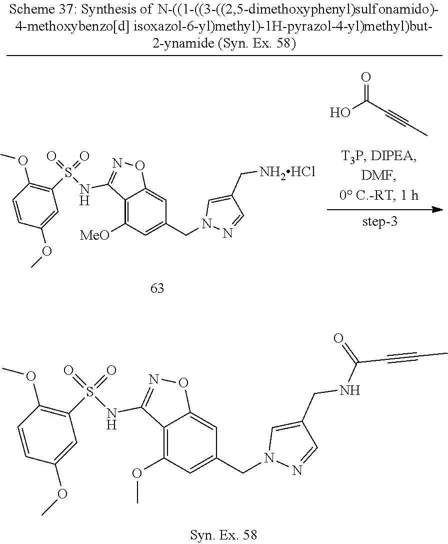

To a stirred solution of compound 63 (0.10 g, 0.196 mmol) in DMF (2 mL) was added DIPEA (0.0066 mL, 0.038 mmol), but-2-ynoic acid 5 (32 mg, 0.392 mmol) and 50% T₃P in EtOAc (0.18 mL, 0.294 mmol) at 0° C. The mixture was stirred at room temperature for 1 h. After completion (monitored by TLC), the mixture was concentrated under reduced pressure. The crude was purified by prep. HPLC to afford title compound N-((1-((3-((2,5-dimethoxyphenyl) sulfonamido)-4-methoxybenzo[d]isoxazol-6-yl)methyl)-1H-pyrazol-4-yl)methyl)but-2-ynamide (Syn. Ex. 58; 16 mg, 15.23%) as an off-white solid. (See analytical data in Table 1).

### Example 59

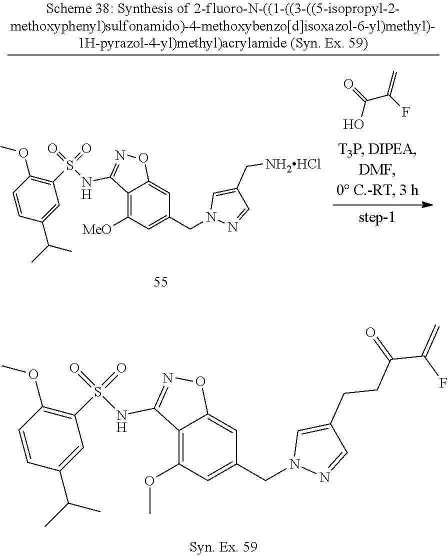

To a stirred solution of compound 55 (0.09 g, 0.172 mmol) in DMF (2 mL) was added DIPEA (0.09 mL, 0.516 mmol), 2-fluoroacrylic acid (30 mg, 0.344 mmol) and 50% T₃P in EtOAc (0.328 mL, 0.516 mmol) at 0° C. The mixture was stirred at room temperature for 3 h. After completion (monitored by TLC), the mixture was quenched with ice-cold water (30 mL) and extracted with EtOAc (3×30 mL). The organic layer was washed with brine, dried over anhydrous Na₂SO₄, and concentrated under reduced pressure. The crude was purified by prep. HPLC to afford title compound 2-fluoro-N-((1-((3-((5-isopropyl-2-methoxyphenyl)sulfonamido)-4-methoxybenzo[d]isoxazol-6-yl)methyl)-1H-pyrazol-4-yl)methyl)acrylamide (Syn. Ex. 59; 23 mg, 23.93%) as an off-white solid. (See analytical data in Table 1).

### Example 60

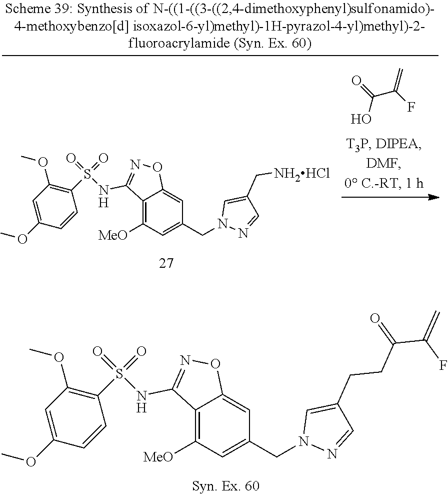

To a stirred solution of compound 60 (0.15 g, 0.294 mmol) in DMF (5 mL) were added DIPEA (0.15 mL, 0.882 mmol), 2-fluoroacrylic acid 2 (52 mg, 0.588 mmol) and 50% T₃P in EtOAc (0.560 mL, 0.882 mmol) at 0° C. The mixture was stirred at room temperature for 1 h. After completion (monitored by TLC), the mixture was quenched with ice-cold water (20 mL) and extracted with EtOAc (3×20 mL). The organic layer was washed with brine, dried over anhydrous Na₂SO₄, filtered and concentrated under reduced pressure. The crude was purified by combi-flash chromatography (80% EtOAc in Hexane) to afford title compound N-((1-((3-((2,4-dimethoxyphenyl)sulfonamido)-4-methoxybenzo[d]isoxazol-6-yl)methyl)-1H-pyrazol-4-yl) methyl)-2-fluoroacrylamide (Syn. Ex. 60; 20 mg, 12.5%) as an off-white solid. (See analytical data in Table 1).

### Example 61

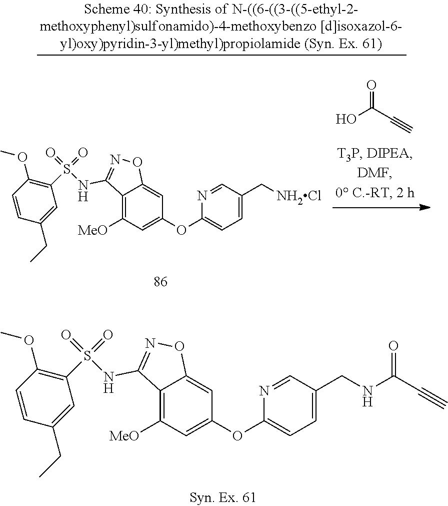

To a stirred solution of compound 86 (0.12 g, 0.230 mmol) in DMF (1 mL) were added propiolic acid (24 mg, 0.345 mmol), DIPEA (0.12 mL, 0.69 mmol) and 50% T₃P in EtOAc (0.292 mL, 0.460 mmol) at 0° C. The mixture was stirred at room temperature for 2 h. After completion (monitored by TLC), the mixture was quenched with ice-cold water and extracted with ethyl acetate. The organic layer was dried over anhydrous Na₂SO₄, filtered and concentrated under reduced pressure. The crude was purified by prep. HPLC to afford title compound N-((6-((3-((5-ethyl-2-methoxyphenyl) sulfonamido)-4-methoxybenzo[d]isoxazol-6-yl)oxy)pyridin-3-yl)methyl)propiolamide (Syn. Ex. 61; 7 mg, 5.83%) as an off-white solid. (See analytical data in Table 1).

### Example 62

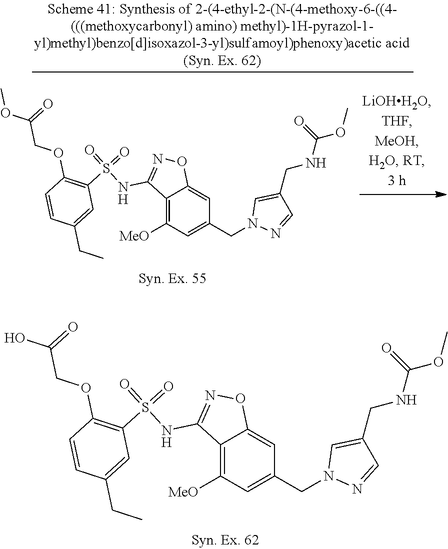

To a stirred solution of Syn. Ex. 55 (5 mg, 0.008 mmol) in mixture of THF (0.5 mL), H₂O (0.5 mL) and MeOH (0.5 mL) was added LiOH·H₂O (0.714 mg, 0.016 mmol) at 0° C. The mixture was stirred at room temperature for 3 h. After completion (monitored by TLC), the mixture was concentrated under reduced pressure, residue was dissolved in water, acidified with citric acid to pH 4-5 and extracted with 10% MeOH in DCM (3×5 mL). The mixture was dried over anhydrous Na₂SO₄, filtered and concentrated under reduced pressure. The crude was triturated with n-pentane (10 mL) to afford title compound 2-(4-ethyl-2-(N-(4-methoxy-6-((4-(((methoxycarbonyl)amino)methyl)-1H-pyrazol-1-yl)methyl)benzo[d]isoxazol-3-yl) sulfamoyl)phenoxy)acetic acid (Syn. Ex. 62; 4.5 mg, 93.75% yield) as an off-white solid. (See analytical data in Table 1).

### Example 63

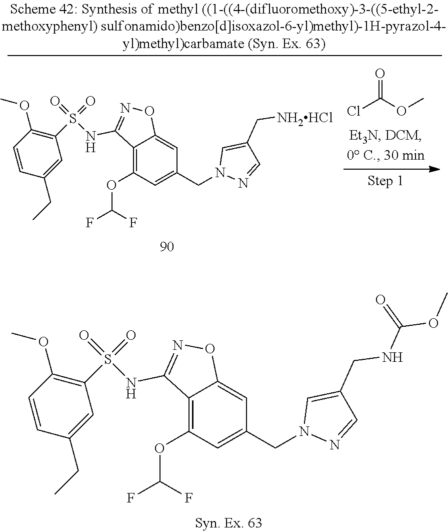

To a stirred solution of compound 90 (0.13 g, 0.238 mmol) in DCM (10 mL) were added Et₃N (0.066 mL, 0.476 mmol) and methyl carbonochloridate (22 mg, 0.238 mmol) in DCM (1 mL) drop-wise at 0° C. The mixture was stirred at 0° C. for 30 min. After completion (monitored by TLC), the mixture was quenched with ice-cold water and extracted with DCM. The organic layer was dried over anhydrous Na₂SO₄, filtered and concentrated under reduced pressure. The crude was purified by prep-HPLC to afford title compound methyl ((1-((4-(difluoromethoxy)-3-((5-ethyl-2-methoxyphenyl)sulfonamido)benzo[d]isoxazol-6-yl)methyl)-1H-pyrazol 4yl)methyl)carbamate (Syn. Ex. 63; 3 mg, 2.22%) as an off-white solid. (See analytical data in Table 1).

### Examples 64-65

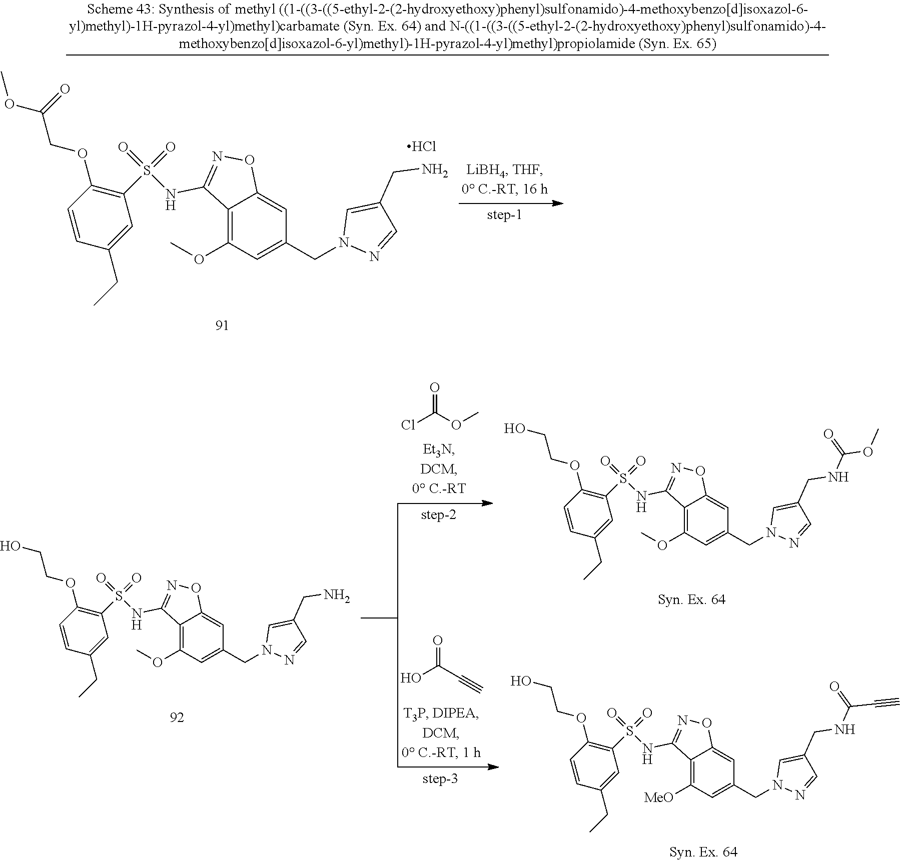

### Synthesis of N-(6-((4-(aminomethyl)-1H-pyrazol-1-yl)methyl)-4-methoxybenzo[d]isoxazol-3-yl)-5-ethyl-2-(2-hydroxyethoxy)benzenesulfonamide hydrochloride (92)

To a stirred solution of compound 91 (0.15 g, 0.265 mmol) in THF (5 mL) was added 2M LiBH₄ solution in THF (0.39 mL, 0.795 mmol) at 0° C. The reaction mixture was stirred at room temperature for 16 h. After completion (monitored by TLC), the mixture was quenched with aqueous NH₄Cl and extracted with 10% MeOH in DCM. The combined organic layer was washed with brine, dried over anhydrous Na₂SO₄, filtered, and concentrated under reduced pressure to afford title compound 92 (6 mg, 24.83%) as a light-yellow solid. LCMS calculated for C23H27N5O6S: 501.17; Found: 502.26 (M+1).

### Synthesis of methyl ((1-((3-((5-ethyl-2-(2-hydroxyethoxy)phenyl)sulfonamido)-4-methoxybenzo[d]isoxazol-6-yl)methyl)-1H-pyrazol-4-yl)methyl)carbamate (Syn. Ex. 64)

To a stirred solution of compound 92 (0.10 g, 0.185 mmol) in DCM (5 mL) was added Et₃N (0.077 mL, 0.557 mmol) and methyl chloroformate (0.03 g, 0.371 mmol) in DCM (0.50 mL) at 0° C. for 1 h while maintaining the pH 7-8. The mixture was stirred at room temperature for 1 h. After completion (monitored by TLC), the mixture was quenched with ice-cold water and extracted with DCM. The combined organic layer was dried over anhydrous Na₂SO₄ and concentrated under reduced pressure. The crude was purified prep. HPLC to afford methyl ((1-((3-((5-ethyl-2-(2-hydroxyethoxy)phenyl)sulfonamido)-4-methoxybenzo[d]isoxazol-6-yl)methyl)-1H-pyrazol-4-yl)methyl)carbamate (Syn. Ex. 64, 8 mg, 3.04%) as an off-white solid. (See analytical data in Table 1).

### Synthesis of N-((1-((3-((5-ethyl-2-(2-hydroxyethoxy)phenyl)sulfonamido)-4-methoxybenzo[d]isoxazol-6-yl)methyl)-1H-pyrazol-4-yl)methyl)propiolamide (Syn. Ex. 65)

To a stirred solution of compound 92 (0.10 g, 0.199 mmol) in DMF (5 mL) were added DIPEA (0.10 mL, 0.598 mmol), propiolic acid (0.02 g, 0.398 mmol) and 50% T₃P in EtOAc (0.38 mL, 0.598 mmol) at 0° C. The reaction mixture was stirred at room temperature for 1 h. After completion (monitored by TLC), the mixture was quenched with ice-cold water and extracted with 10% MeOH in DCM. The combined organic layer was dried over anhydrous Na₂SO₄ and concentrated under reduced pressure. The crude was purified by prep. HPLC to afford N-((1-((3-((5-ethyl-2-(2-hydroxyethoxy)phenyl)sulfonamido)-4-methoxybenzo[d]isoxazol-6-yl)methyl)-1H-pyrazol-4-yl)methyl)propiolamide (Syn. Ex. 65; 5 mg, 4.44%) as an off-white solid. (See analytical data in Table 1).

### Example 66

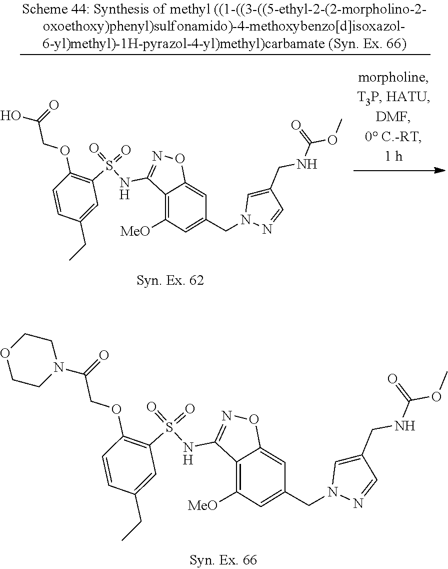 [SMILES: CCc1ccc(OCC(=O)O)c(S(=O)(=O)Nc2noc3cc(Cn4cc(CNC(=O)OC)cn4)cc(OC)c23)c1 ; CCc1ccc(OCC(=O)N2CCOCC2)c(S(=O)(=O)Nc2noc3cc(Cn4cc(CNC(=O)OC)cn4)cc(OC)c23)c1]

To a stirred solution of Syn. Ex. 62 (0.10 g, 0.174 mmol) in DMF (5 mL) were added DIPEA (0.091 mL, 0.523 mmol), morpholine (0.03 g, 0.348 mmol) followed by 50% T₃P in EtOAc (0.33 mL, 0.523 mmol) at 0° C. The reaction mixture was stirred at room temperature for 1 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford methyl ((1-((3-((5-ethyl-2-(2-morpholino-2-oxoethoxy)phenyl)sulfonamido)-4-methoxybenzo[d]isoxazol-6-yl)methyl)-1H-pyrazol-4-yl)methyl)carbamate (Syn. Ex. 66; 18 mg, 15.83%) as an off-white solid. (See analytical data in Table 1).

### Examples 67-68

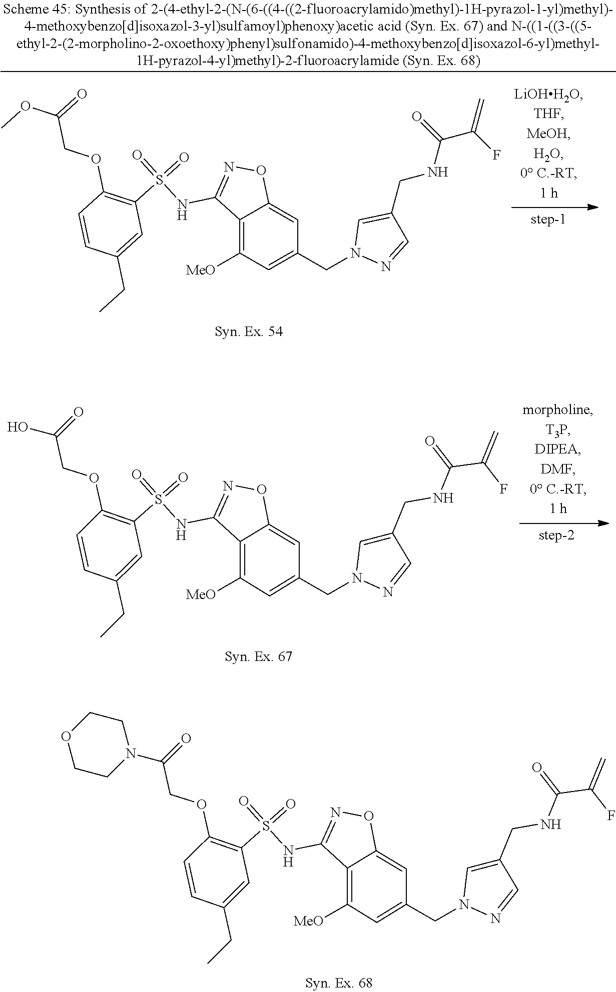 [SMILES: C=C(F)C(=O)NCc1cnn(Cc2cc(OC)c3c(NS(=O)(=O)c4cc(CC)ccc4OCC(=O)OC)noc3c2)c1 ; C=C(F)C(=O)NCc1cnn(Cc2cc(OC)c3c(NS(=O)(=O)c4cc(CC)ccc4OCC(=O)O)noc3c2)c1 ; C=C(F)C(=O)NCc1cnn(Cc2cc(OC)c3c(NS(=O)(=O)c4cc(CC)ccc4OCC(=O)N4CCOCC4)noc3c2)c1]

### Synthesis of 2-(4-ethyl-2-(N-(6-((4-((2-fluoroacrylamido)methyl)-1H-pyrazol-1-yl)methyl)-4-methoxybenzo[d]isoxazol-3-yl)sulfamoyl)phenoxy)acetic acid (Syn. Ex. 67)

To a stirred solution of Syn. Ex. 54 (0.20 g, 0.332 mmol) in mixture of THF:MeOH : H₂O (2:2:2; 6 mL) was added LiOH·H₂O (0.069 g, 1.662 mmol) at 0° C. The reaction mixture was stirred at room temperature for 1 h. After completion (monitored by TLC), the mixture was concentrated under reduced pressure, diluted with ice-cold water and acidified with citric acid to pH 4-5. The aqueous layer was extracted with 10% MeOH in DCM, dried over anhydrous Na₂SO₄, filtered and concentrated under reduced pressure to afford crude (0.15 g). The crude (30 mg) was purified by prep. HPLC to afford 2-(4-ethyl-2-(N-(6-((4-((2-fluoroacrylamido)methyl)-1H-pyrazol-1-yl)methyl)-4-methoxybenzo[d]isoxazol-3-yl)sulfamoyl)phenoxy)acetic acid (Syn. Ex. 67; 5.00 mg) as an off-white solid. (See analytical data in Table 1).

### Synthesis of N-((1-((3-((5-ethyl-2-(2-morpholino-2-oxoethoxy)phenyl)sulfonamido)-4-methoxybenzo[d]isoxazol-6-yl)methyl)-1H-pyrazol-4-yl)methyl)-2-fluoroacrylamide (Syn. Ex. 68)

To a stirred solution of compound Syn. Ex. 67 (0.10 g, 0.170 mmol) in DMF (5 mL) were added DIPEA (0.089 mL, 0.510 mmol), morpholine (0.029 g, 0.340 mmol) and 50% T₃P in EtOAc (0.32 mL, 0.510 mmol) at 0° C. The mixture was stirred at room temperature for 1 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-((1-((3-((5-ethyl-2-(2-morpholino-2-oxoethoxy)phenyl)sulfonamido)-4-methoxybenzo[d]isoxazol-6-yl)methyl)-1H-pyrazol-4-yl)methyl)-2-fluoroacrylamide (Syn. Ex. 68; 6 mg, 5.30%) as an off-white solid. (See analytical data in Table 1).

### Example 69

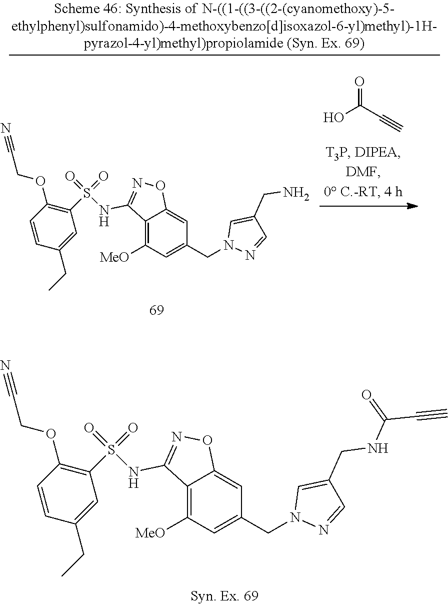

To a stirred solution of compound 69 (0.10 g, 0.187 mmol) in DMF (2 mL) were added DIPEA (0.098 mL, 0.562 mmol), propiolic acid (0.026 g, 0.375 mmol) and 50% T₃P in EtOAc (0.35 mL, 0.562 mmol) at 0° C. The reaction mixture was stirred at room temperature for 4 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-((1-((3-((2-(cyanomethoxy)-5-ethylphenyl)sulfonamido)-4-methoxybenzo[d]isoxazol-6-yl)methyl)-1H-pyrazol-4-yl)methyl)propiolamide (Syn. Ex. 69; 5 mg, 4.81%) as an off-white solid. (See analytical data in Table 1).

### Examples 70-72

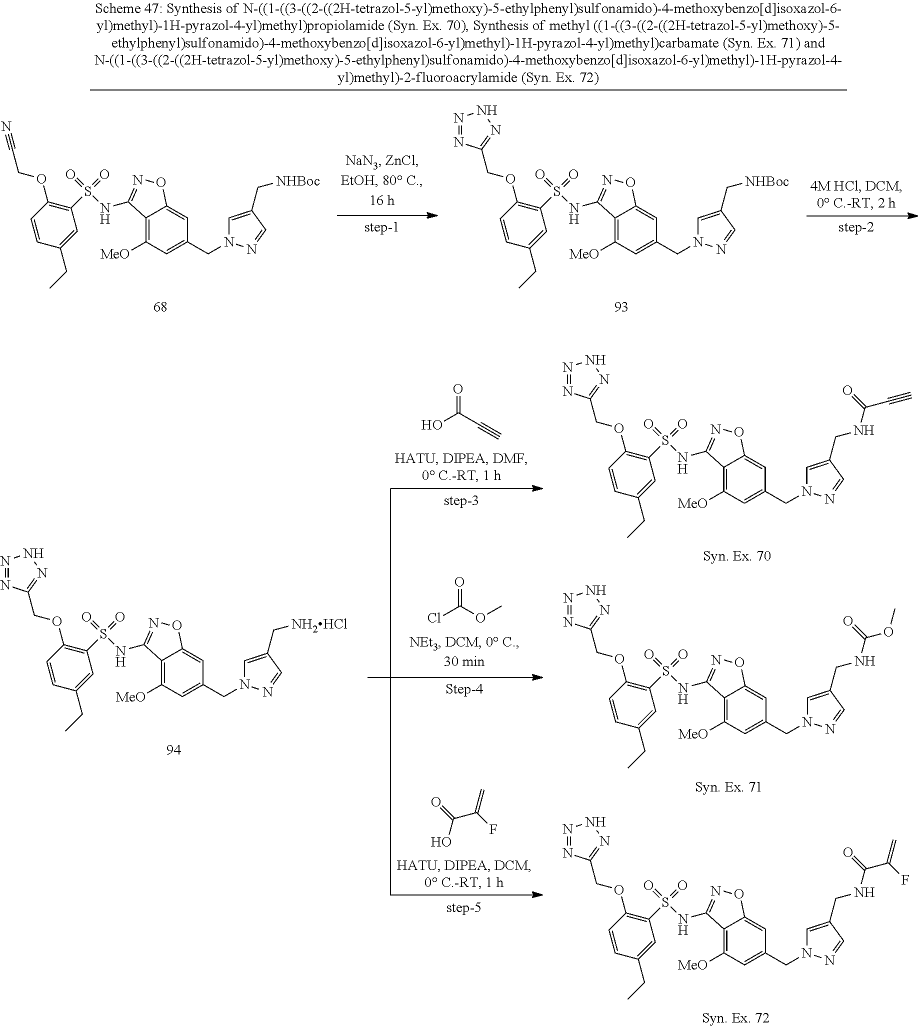

### Synthesis of tert-Butyl ((1-((3-((2-((2H-tetrazol-5-yl)methoxy)-5-ethylphenyl)sulfonamido)-4-methoxybenzo[d]isoxazol-6-yl)methyl)-1H-pyrazol-4-yl)methyl)carbamate (93)

To a stirred solution of compound 68 (20 mg, 0.033 mmol) in EtOH (1 mL) was added ZnCl (6.8 mg, 0.050 mmol) and NaN₃ (3.2 mg, 0.050 mmol) at room temperature. The reaction mixture was stirred at 80° C. for 16 h. After completion (monitored by TLC), the mixture was filtered through a celite pad. The organic layer was dried over anhydrous Na₂SO₄, filtered and concentrated under reduced pressure to afford title compound 93 (15 mg, 45.12%) as a yellow solid. LCMS calculated for C28H33N907S: 639.22; Found: 640.20 (M+1).

### Synthesis of 2-((2H-tetrazol-5-yl)methoxy)-N-(6-((4-(aminomethyl)-1H-pyrazol-1-yl)methyl)-4-methoxybenzo[d]isoxazol-3-yl)-5-ethylbenzenesulfonamide hydrochloride (94)

To a stirred solution of compound 93 (0.15 g, 0.234 mmol) in DCM (5 mL) was added 4M HCl in 1,4-Dioxane (0.29 mL, 1.172 mmol) at 0° C. The reaction mixture was stirred at room temperature for 2 h. After completion (monitored by TLC), the mixture was concentrated under reduced pressure. The crude was triturated with DCM, co-distilled with Et₂O and concentrated under reduced pressure to title compound 94 (0.14 g, 73.10%) as an off-white solid. LCMS calculated for C23H26ClN905S: 539.17; Found: 538.20 (M−1).

### Synthesis of N-((1-((3-((2-((2H-tetrazol-5-yl)methoxy)-5-ethylphenyl)sulfonamido)-4-methoxybenzo[d]isoxazol-6-yl)methyl)-1H-pyrazol-4-yl)methyl)propiolamide (Syn. Ex. 70)

To a stirred solution of compound 94 (70 mg, 0.121 mmol) in DMF (4 mL) were added DIPEA (0.063 mL, 0.364 mmol), propiolic acid (0.01 g, 0.145 mmol) and HATU (0.072 g, 0.182 mmol) at 0° C. The reaction mixture was stirred at room temperature for 1 h. After completion (monitored by TLC), the mixture was quenched with ice-cold water and extracted with EtOAc. The combined organic layer was dried over anhydrous Na₂SO₄, filtered and concentrated under reduced pressure. The crude was purified by prep. HPLC to afford N-((1-((3-((2-((2H-tetrazol-5-yl)methoxy)-5-ethylphenyl)sulfonamido)-4-methoxybenzo[d]isoxazol-6-yl)methyl)-1H-pyrazol-4-yl)methyl)propiolamide (Syn. Ex. 70; 7 mg, 9.53%) as an off-white solid. (See analytical data in Table 1).

### Synthesis of methyl ((1-((3-((2-((2H-tetrazol-5-yl)methoxy)-5-ethylphenyl)sulfonamido)-4-methoxybenzo[d]isoxazol-6-yl)methyl)-1H-pyrazol-4-yl)methyl)carbamate (Syn. Ex. 71)

To a stirred solution of compound 94 (60 mg, 0.104 mmol) in DCM (4 mL) was added NEt₃ (0.029 mL, 0.208 mmol) and methyl chloroformate (12 mg, 0.125 mmol) at 0° C. The reaction mixture was stirred at 0° C. for 30 min. After completion (monitored by TLC), the mixture was quenched with ice-cold water and extracted with DCM. The combined organic layer was dried over anhydrous Na₂SO₄, filtered and concentrated under reduced pressure. The crude was purified prep. HPLC to afford methyl ((1-((3-((2-((2H-tetrazol-5-yl)methoxy)-5-ethylphenyl)sulfonamido)-4-methoxybenzo[d]isoxazol-6-yl)methyl)-1H-pyrazol-4-yl)methyl)carbamate (Syn. Ex. 71; 5 mg, 7.98%) as an off-white solid. (See analytical data in Table 1).

### Synthesis of N-((1-((3-((2-((2H-tetrazol-5-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 72)

To a stirred solution of compound 94 (0.12 g, 0.208 mmol) in DCM (6 mL) were added 2-fluoroacrylic acid (0.02 g, 0.328 mmol), DIPEA (0.14 mL, 0.850 mmol) and HATU (0.16 g, 0.429 mmol) at 0° C. The reaction mixture was stirred at room temperature for 1 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-((1-((3-((2-((2H-tetrazol-5-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 72; 14.6 mg, 11.23%) as an off-white solid. (See analytical data in Table 1).

### Examples 73-74

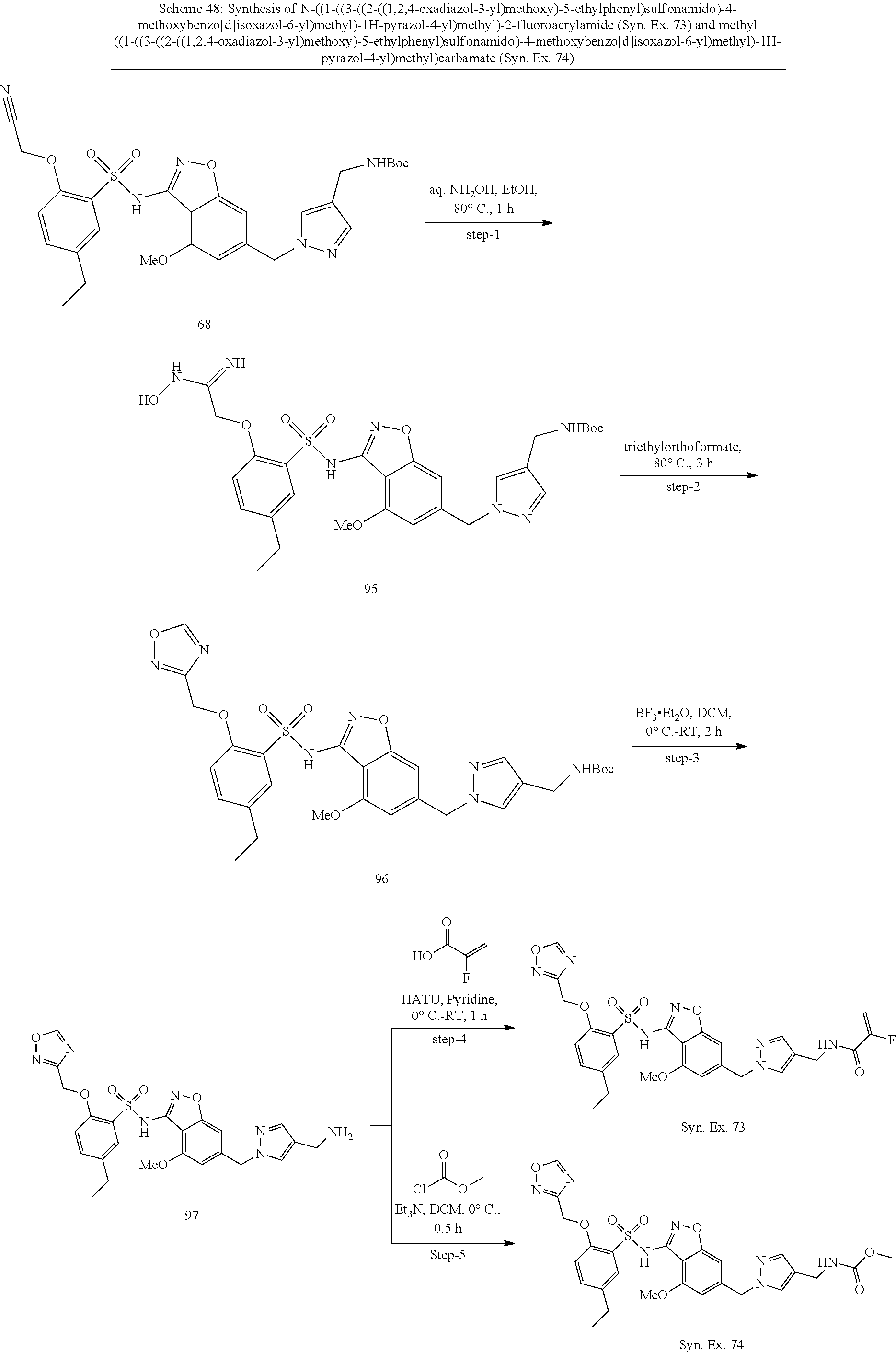

### Synthesis of tert-Butyl ((1-((3-((5-ethyl-2-(2-(hydroxyamino)-2-iminoethoxy)phenyl)sulfonamido)-4-methoxybenzo[d]isoxazol-6-yl)methyl)-1H-pyrazol-4-yl)methyl)carbamate (95)

To a stirred solution of compound 68 (0.30 g, 0.502 mmol) in EtOH (10 mL) was added 50% aqueous NH₂OH (0.49 mL, 16.361 mmol). The reaction mixture was stirred at 80° C. for 1 h. After completion (monitored by TLC), the mixture was quenched with ice-cold water and extracted with EtOAc. The combined organic layer was washed with brine, dried over anhydrous Na₂SO₄ and concentrated under reduced pressure to afford title compound 95 (0.30 g, 94.75%) as an off-white solid. LCMS calculated for C28H35N708S: 629.23; Found: 630.30 (M+1).

### Synthesis of tert-Butyl ((1-((3-((2-((1,2,4-oxadiazol-3-yl)methoxy)-5-ethylphenyl)sulfonamido)-4-methoxybenzo[d]isoxazol-6-yl)methyl)-1H-pyrazol-4-yl)methyl)carbamate (96)

A solution of compound 95 (0.30 g, 0.476 mmol) in triethylorthoformate (10 mL) was stirred at 80° C. for 3 h. After completion (monitored by TLC), the mixture was cooled at room temperature and concentrated under reduced pressure to afford title compound 96 (0.30 g, 98.45%) as an off-white solid. LCMS calculated for C29H33N708S: 639.21; Found: 638.30 (M−1).

### Synthesis of 2-((1,2,4-oxadiazol-3-yl)methoxy)-N-(6-((4-(aminomethyl)-1H-pyrazol-1-yl)methyl)-4-methoxybenzo[d]isoxazol-3-yl)-5-ethylbenzenesulfonamide (97)

To a stirred solution of compound 96 (0.28 g, 0.437 mmol) in DCM (50 mL) was added BF₃·Et₂O (0.50 mL) at 0° C. The reaction mixture was stirred at room temperature for 2 h. After completion (monitored by TLC), the mixture was concentrated under reduced pressure triturated with Et₂O to afford title compound 97 (0.28 g, 67.58%) as an off-white solid. LCMS calculated for C24H25N706S: 539.16; Found: 540.30 (M+1).

### Synthesis of N-((1-((3-((2-((1,2,4-oxadiazol-3-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 73)

To a stirred solution of 2-fluoroacrylic acid (0.02 g, 0.222 mmol) in pyridine (5 mL) were added HATU (0.14 g, 0.370 mmol) and compound 97 (0.10 g, 0.185 mmol) at 0° C. The reaction mixture was stirred at room temperature for 1 h. After completion (monitored by TLC), the mixture was quenched with water and extracted with EtOAc. The combined organic layer was dried over anhydrous Na₂SO₄, filtered and concentrated under reduced pressure. The crude was purified by prep. HPLC to afford N-((1-((3-((2-((1,2,4-oxadiazol-3-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 73; 48 mg, 41.95%) as an off-white solid. (See analytical data in Table 1).

### Synthesis of methyl ((1-((3-((2-((1,2,4-oxadiazol-3-yl)methoxy)-5-ethylphenyl)sulfonamido)-4-methoxybenzo[d]isoxazol-6-yl)methyl)-1H-pyrazol-4-yl)methyl)carbamate (Syn. Ex. 74)

To a stirred solution of compound 97 (0.10 g, 0.185 mmol) in DCM (5 mL) were added NEt₃ (0.05 mL, 0.370 mmol) and methyl chloroformate (0.01 g, 0.185 mmol) at 0° C. The reaction mixture was stirred at 0° C. for 30 min. After completion (monitored by TLC), the mixture was quenched with water and extracted with DCM. The combined organic layer was dried over anhydrous Na₂SO₄, filtered and concentrated under reduced pressure. The crude was purified by prep. HPLC to afford methyl ((1-((3-((2-((1,2,4-oxadiazol-3-yl)methoxy)-5-ethylphenyl)sulfonamido)-4-methoxybenzo[d]isoxazol-6-yl)methyl)-1H-pyrazol-4-yl)methyl)carbamate (Syn. Ex. 74; 4.00 mg, 3.50%) as an off-white solid. (See analytical data in Table 1).

### Example 75

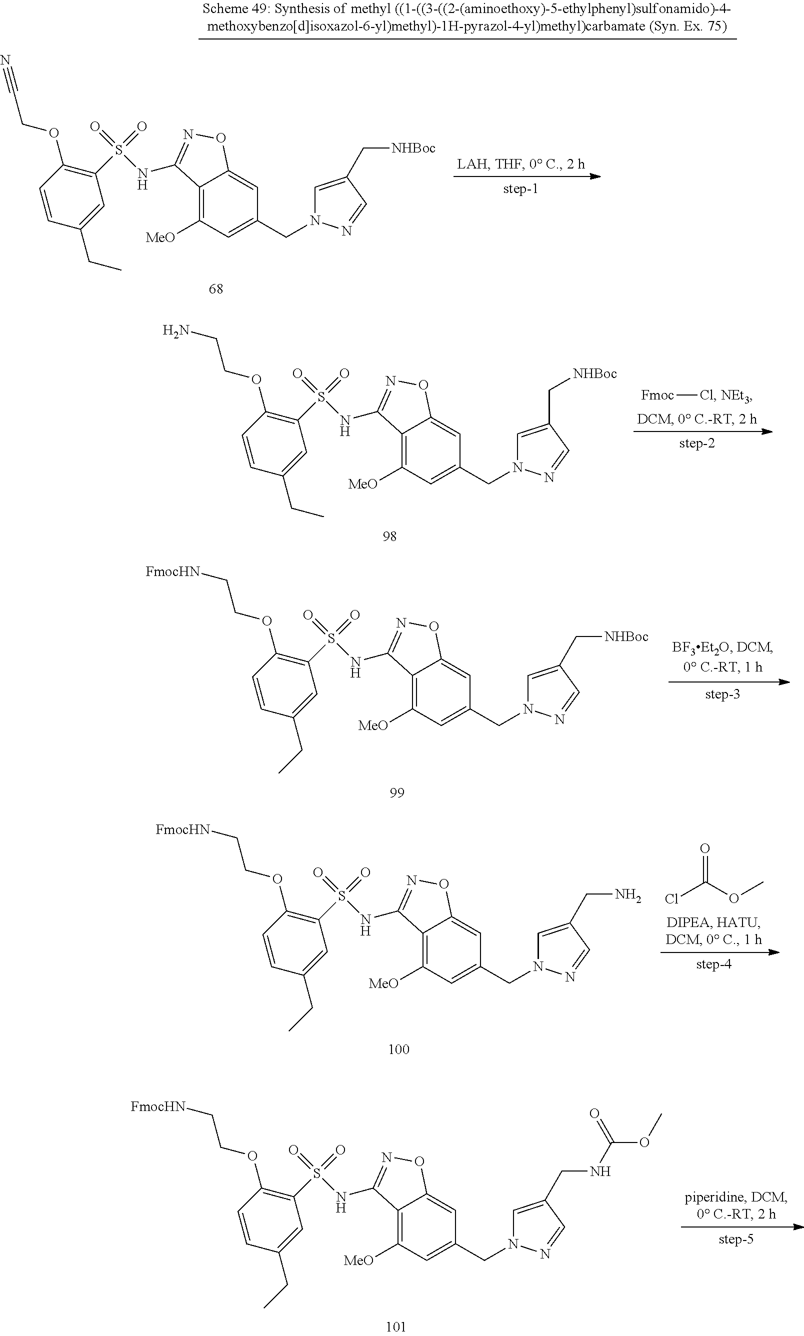

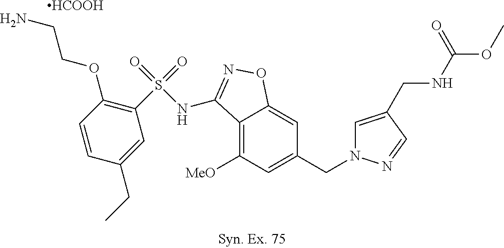 [SMILES: "·HCOOH" ; CCc1ccc(OCCN)c(S(=O)(=O)Nc2noc3cc(Cn4cc(CNC(=O)OC)cn4)cc(OC)c23)c1]

### Synthesis of tert-Butyl ((1-((3-((2-(2-aminoethoxy)-5-ethylphenyl)sulfonamido)-4-methoxybenzo[d]isoxazol-6-yl)methyl)-1H-pyrazol-4-yl)methyl)carbamate (98)

To a stirred solution of compound 68 (1 g, 1.675 mmol) in THF (10 mL) were added 2M LAH in THF (0.83 mL, 1.675 mmol) at 0° C. The reaction mixture was stirred at 0° C. for 2 h. After completion (monitored by TLC), the mixture was quenched with ice-cold NH₄Cl and extracted with 10% MeOH in DCM. The organic layer was dried over anhydrous Na₂SO₄, concentrated under reduced pressure and purified by combi-flash chromatography using 5-10% MeOH/DCM to afford title compound 98 (0.36 g, 27.72%) as an off-white solid. LCMS calculated for C28H36N6O7S: 600.24; Found: 601.30 (M+1).

### Synthesis of (9H-fluoren-9-yl)methyl (2-(2-(N-(6-((4-(((tert-butoxycarbonyl)amino)methyl)-1H-pyrazol-1-yl)methyl)-4-methoxybenzo[d]isoxazol-3-yl)sulfamoyl)-4-ethylphenoxy)ethyl)carbamate (99)

To a stirred solution of compound 98 (0.23 g, 0.382 mmol) in DCM (10 mL) were added NEt₃ (0.16 mL, 1.148 mmol) and Fmoc-C₁ (0.12 g, 0.459 mmol) at 0° C. The reaction mixture was stirred at room temperature for 2 h. After completion (monitored by TLC), the mixture was quenched with ice-cold water, extracted with DCM, dried over anhydrous Na₂SO₄ and concentrated under reduced pressure. The crude was triturated with Et₂O to afford title compound 99 (0.31 g, 65.92%) as a pale-yellow solid. LCMS calculated for C₄₃H46N6O9S: 822.30; Found: 723.30 [(M-Boc)+1].

### Synthesis of (9H-fluoren-9-yl)methyl (2-(2-(N-(6-((4-(aminomethyl)-1H-pyrazol-1-yl)methyl)-4-methoxybenzo[d]isoxazol-3-yl)sulfamoyl)-4-ethylphenoxy)ethyl)carbamate (100)

To a stirred solution of compound 99 (0.31 g, 0.376 mmol) in DCM (5 mL) was added BF₃·Et₂O (0.16 g, 0.565 mmol) at 0° C. The reaction mixture was stirred at room temperature for 1 h. After completion (monitored by TLC), the mixture was concentrated under reduced pressure. The crude was triturated with Et₂O and dried under reduced pressure to afford title compound 100 (0.29 g, 31.95%) as a pale-yellow solid. LCMS calculated for C38H38N6O7S: 722.25; Found: 723.30 (M+1).

### Synthesis of methyl ((1-((3-((2-(2-((((9H-fluoren-9-yl)methoxy)carbonyl)amino)ethoxy)-5-ethylphenyl)sulfonamido)-4-methoxybenzo[d]isoxazol-6-yl)methyl)-1H-pyrazol-4-yl)methyl)carbamate (101)

To a stirred solution of compound 100 (0.14 g, 0.193 mmol) in DCM (5 mL) were added NEt₃ (0.08 mL, 0.581 mmol) and methyl chloroformate (0.03 g, 0.387 mmol) in DCM (0.50 mL) at 0° C. The reaction mixture was stirred at room temperature for 1 h. After completion (monitored by TLC), the mixture was quenched with ice-cold water and extracted with 10% MeOH in DCM. The combined organic layer was dried over anhydrous Na₂SO₄ and concentrated under reduced pressure to afford title compound 101 (0.17 g, 49.34%) as an off-white gummy solid. LCMS calculated for C40H40N6O9S: 780.26; Found: 781.30 (M+1).

### Synthesis of methyl ((1-((3-((2-(2-aminoethoxy)-5-ethylphenyl)sulfonamido)-4-methoxybenzo[d]isoxazol-6-yl)methyl)-1H-pyrazol-4-yl)methyl)carbamate (Syn. Ex. 75)

To a stirred solution of compound 101 (0.14 g, 0.179 mmol) in DCM (5 mL) was added piperidine (0.03 g, 0.358 mmol) at 0° C. The reaction mixture was stirred at room temperature for 2 h. After completion (monitored by TLC), the mixture was quenched with ice-cold water and extracted with 10% MeOH in DCM. The combined organic layer was dried over anhydrous Na₂SO₄ and concentrated under reduced pressure. The crude was purified by prep. HPLC to afford methyl ((1-((3-((2-(2-aminoethoxy)-5-ethylphenyl)sulfonamido)-4-methoxybenzo[d]isoxazol-6-yl)methyl)-1H-pyrazol-4-yl)methyl)carbamate (Syn. Ex. 75; 6 mg, 5.89%) as an off-white solid. (See analytical data in Table 1).

### Example 76

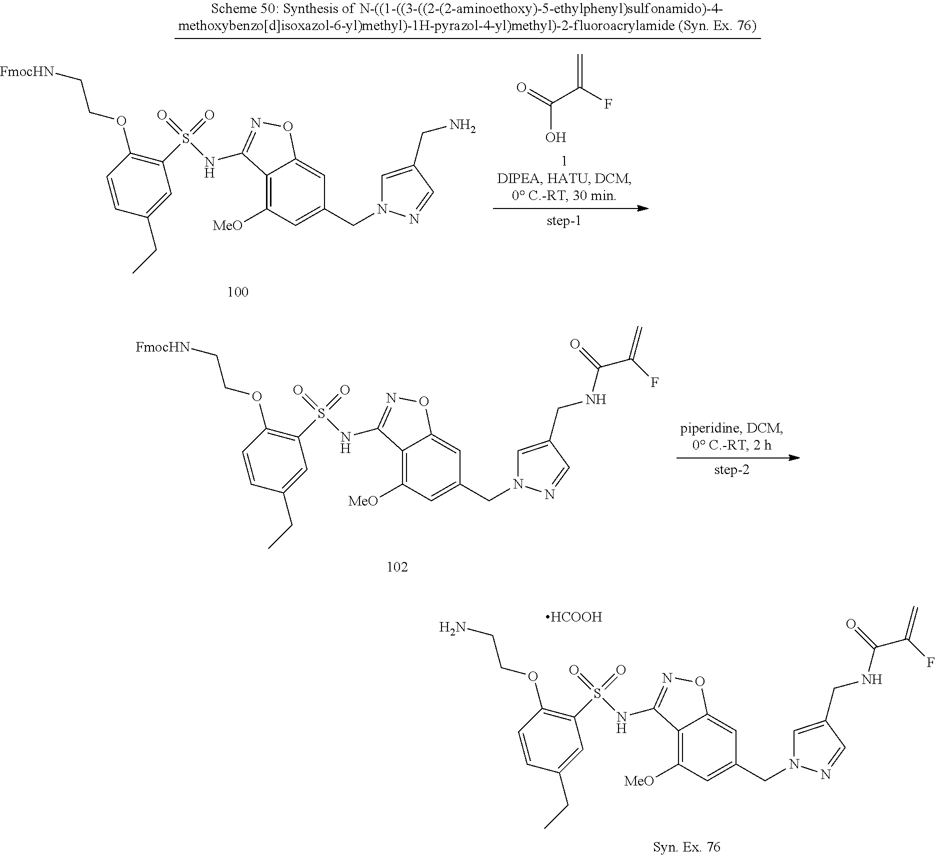

### Synthesis of (9H-fluoren-9-yl)methyl (2-(4-ethyl-2-(N-(6-((4-((2-fluoroacrylamido)methyl)-1H-pyrazol-1-yl)methyl)-4-methoxybenzo[d]isoxazol-3-yl)sulfamoyl)phenoxy)ethyl)carbamate (102)

To a stirred solution of 2-fluoroacrylic acid (0.03 g, 0.415 mmol) in DCM (5 mL) were added DIPEA (0.18 mL, 1.037 mmol) and HATU (0.11 g, 0.311 mmol) followed by the addition of compound 100 (0.15 g, 0.207 mmol) at 0° C. The reaction mixture was stirred at room temperature for 30 min. After completion (monitored by TLC), the mixture was quenched with ice-cold water and extracted with 10% MeOH in DCM. The combined organic layer was dried over anhydrous Na₂SO₄ and concentrated under reduced pressure to afford title compound 102 (0.20 g, 36.37%) as a brown gummy solid. LCMS calculated for C₄₁H39FN6O8S: 794.25; Found: 795.40 (M+1).

### Synthesis of N-((1-((3-((2-(2-aminoethoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 76)

To a stirred solution of compound 102 (0.15 g, 0.188 mmol) in DCM (5 mL) was added piperidine (0.03 g, 0.377 mmol) at 0° C. The reaction mixture was stirred at room temperature for 2 h. After completion (monitored by TLC), the mixture was quenched with ice-cold water and extracted with 10% MeOH in DCM. The combined organic layer was dried over anhydrous Na₂SO₄ and concentrated under reduced pressure. The crude was purified by prep. HPLC to afford N-((1-((3-((2-(2-aminoethoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 76; 12 mg, 11.07%) as an off-white solid. (See analytical data in Table 1).

### Examples 77-78

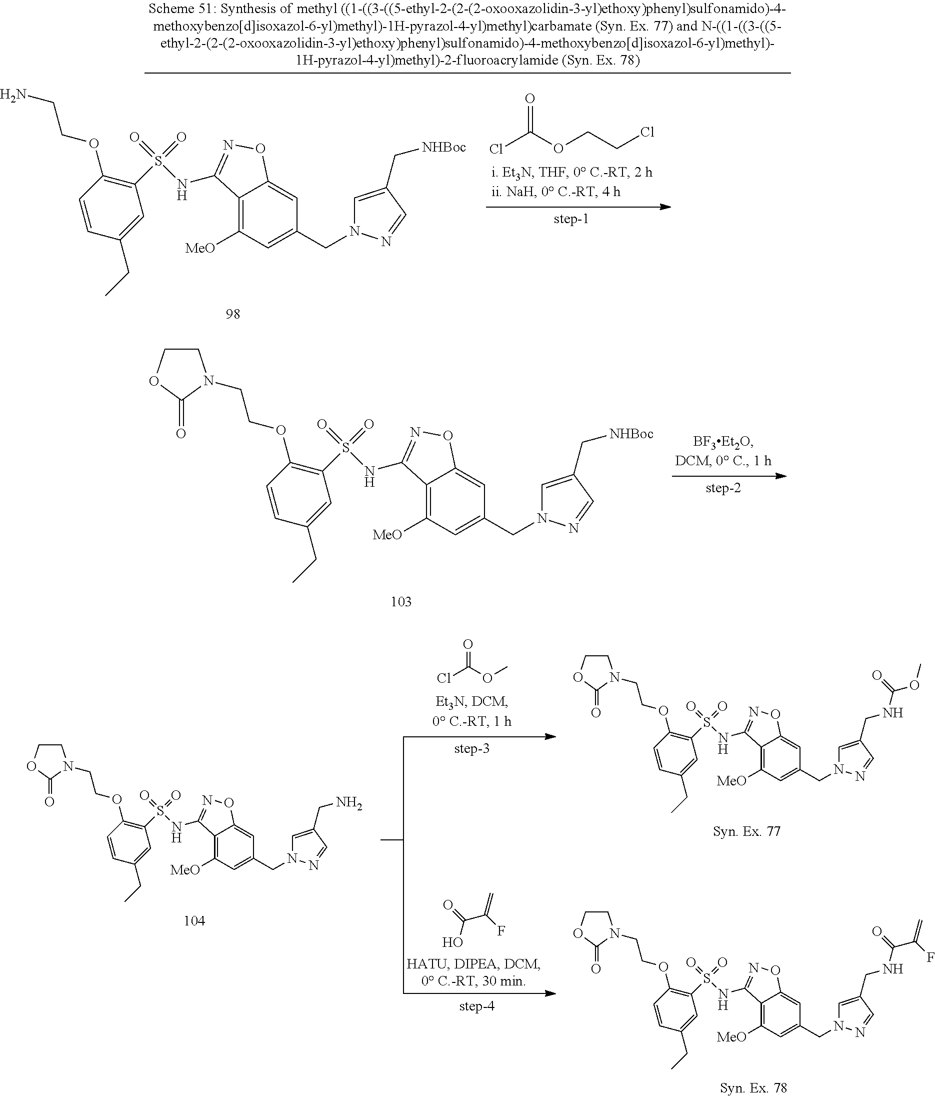

### Synthesis of tert-Butyl ((1-((3-((5-ethyl-2-(2-(2-oxooxazolidin-3-yl)ethoxy)phenyl)sulfonamido)-4-methoxybenzo[d]isoxazol-6-yl)methyl)-1H-pyrazol-4-yl)methyl)carbamate (103)

To a stirred solution of compound 98 (0.30 g, 0.499 mmol) in THF (20 mL) were added NEt₃ (0.27 mL, 1.997 mmol) and 2-chloroethyl chloroformate (0.10 g, 0.749 mmol) at 0° C. The reaction mixture was stirred at room temperature for 2 h followed by addition of 60% NaH in oil (0.19 g, 4.994 mmol) was at 0° C. The reaction mixture was stirred at room temperature for 4 h. After completion (monitored by TLC), the mixture was quenched with ice-cold water and extracted with 10% MeOH in DCM. The combined organic layer was dried over anhydrous Na₂SO₄, filtered and concentrated under reduced pressure. The crude was purified by combi-flash chromatography using 5-10% MeOH/DCM to afford title compound 103 (0.19 g, 39.70%) as a pale-yellow solid. LCMS calculated for C₃₁H38N6O9S: 670.24; Found: 669.40 (M−1).

### Synthesis of N-(6-((4-(aminomethyl)-1H-pyrazol-1-yl)methyl)-4-methoxybenzo[d]isoxazol-3-yl)-5-ethyl-2-(2-(2-oxooxazolidin-3-yl)ethoxy)benzenesulfonamide (104)

To a stirred solution of compound 103 (0.18 g, 0.268 mmol) in DCM (5 mL) was added BF₃·Et₂O (0.15 g, 0.536 mmol) at 0° C. The reaction mixture was stirred at 0° C. for 1 h. After completion (monitored by TLC), the mixture was concentrated under reduced pressure. The crude was triturated with Et₂O and dried under reduced pressure to afford title compound 104 (0.20 g) as a light-yellow solid. LCMS calculated for C26H30N6O7S: 570.19; Found: 571.40 (M+1).

### Synthesis of methyl ((1-((3-((5-ethyl-2-(2-(2-oxooxazolidin-3-yl)ethoxy)phenyl)sulfonamido)-4-methoxybenzo[d]isoxazol-6-yl)methyl)-1H-pyrazol-4-yl)methyl)carbamate (Syn. Ex. 77)

To a stirred solution of compound 104 (0.10 g, 0.175 mmol) in DCM (5 mL) were added NEt₃ (0.07 mL, 0.525 mmol) and methyl chloroformate (0.033 mL, 0.350 mmol) in DCM (0.5 mL) at 0° C. The reaction mixture was stirred at room temperature for 1 h. After completion (monitored by TLC), the mixture was quenched with ice-cold water and extracted with DCM. The combined organic layer was dried over anhydrous Na₂SO₄ and concentrated under reduced pressure. The crude was purified by prep. HPLC to afford methyl ((1-((3-((5-ethyl-2-(2-(2-oxooxazolidin-3-yl)ethoxy)phenyl)sulfonamido)-4-methoxybenzo[d]isoxazol-6-yl)methyl)-1H-pyrazol-4-yl)methyl)carbamate (Syn. Ex. 77; 24 mg, 22.94%) as an off-white solid. (See analytical data in Table 1).

### Synthesis of N-((1-((3-((5-ethyl-2-(2-(2-oxooxazolidin-3-yl)ethoxy)phenyl)sulfonamido)-4-methoxybenzo[d]isoxazol-6-yl)methyl)-1H-pyrazol-4-yl)methyl)-2-fluoroacrylamide (Syn. Ex. 78)

To a stirred solution of 2-fluoroacrylic acid (0.028 g, 0.315 mmol) in DCM (1 mL) were added DIPEA (0.13 mL, 0.788 mmol) and HATU (0.09 g, 0.236 mmol) and 104 (0.09 g, 0.157 mmol) at 0° C. The reaction mixture was stirred at room temperature for 30 min. After completion (monitored by TLC), the mixture was quenched with ice-cold water and extracted with 10% MeOH in DCM. The combined organic layer was dried over anhydrous Na₂SO₄ and concentrated under reduced pressure. The crude was purified by prep. HPLC to afford N-((1-((3-((5-ethyl-2-(2-(2-oxooxazolidin-3-yl)ethoxy)phenyl)sulfonamido)-4-methoxybenzo[d]isoxazol-6-yl)methyl)-1H-pyrazol-4-yl)methyl)-2-fluoroacrylamide (Syn. Ex. 78; 15.5 mg, 15.22%) as an off-white solid. (See analytical data in Table 1).

### Example 79

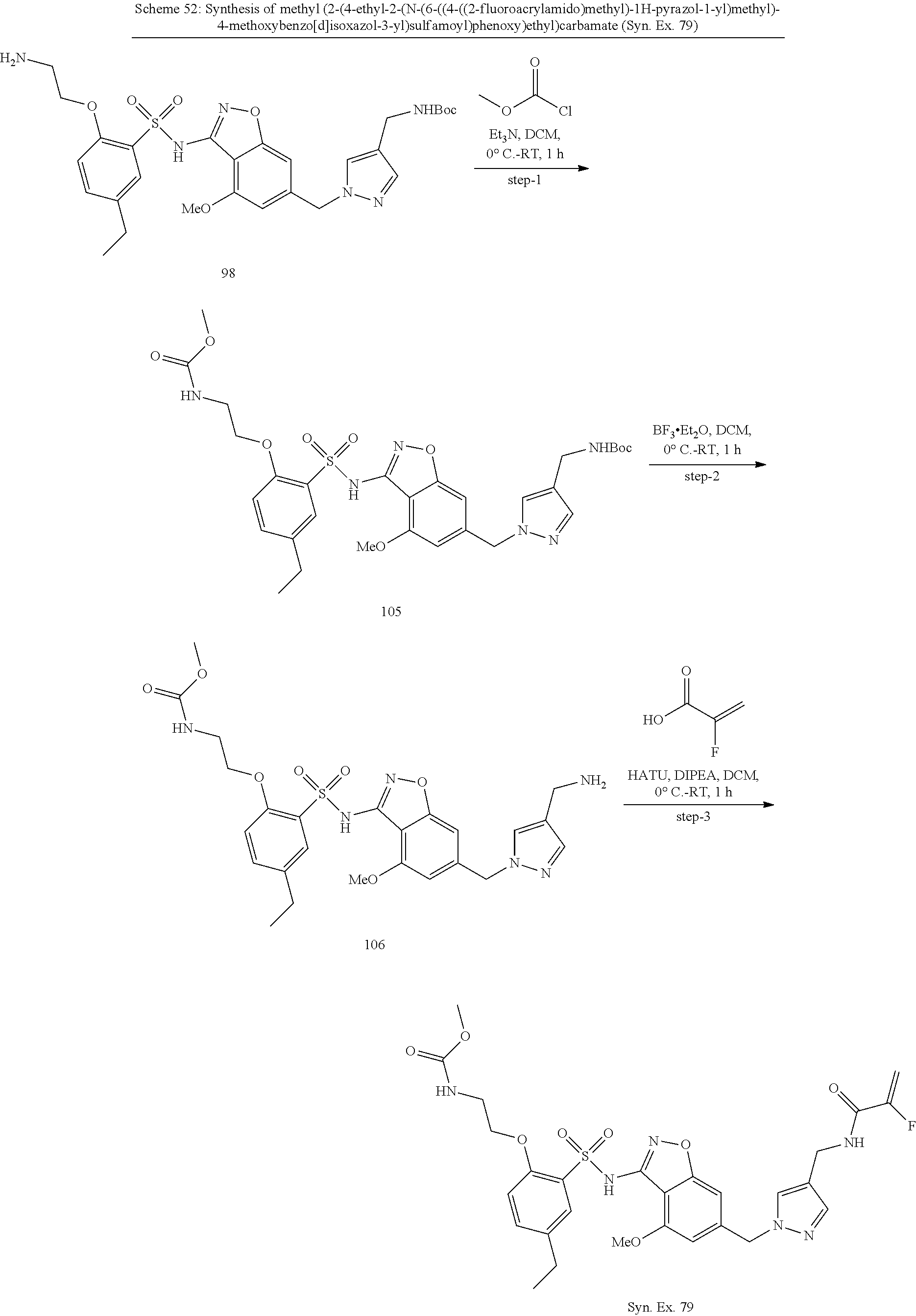

### Synthesis of tert-Butyl ((1-((3-((5-ethyl-2-(2-((methoxycarbonyl)amino)ethoxy)phenyl)sulfonamido)-4-methoxybenzo[d]isoxazol-6-yl)methyl)-1H-pyrazol-4-yl)methyl)carbamate (105)

To a stirred solution of compound 98 (0.20 g, 0.332 mmol) in DCM (5 mL) were added Et₃N (0.13 mL, 0.998 mmol) and methyl chloroformate (0.06 g, 0.665 mmol) at 0° C. The reaction mixture was stirred at 0° C. for 1 h. After completion (monitored by TLC), the mixture was quenched with ice-cold water and extracted with 10% MeOH in DCM. The combined organic layer was dried over anhydrous Na₂SO₄ and concentrated under reduced pressure. The crude was triturated with Et₂O to afford title compound 105 (0.20 g, 75.30%) as a pale-yellow solid. LCMS calculated for C30H38N6O9S: 658.24; Found: 657.30 (M−1).

### Synthesis of methyl (2-(2-(N-(6-((4-(aminomethyl)-1H-pyrazol-1-yl)methyl)-4-methoxybenzo[dlisoxazol-3-yl)sulfamoyl)-4-ethylphenoxy)ethyl)carbamate (106)

To a stirred solution of compound 105 (0.19 g, 0.288 mmol) in DCM (5 mL) was added BF₃·Et₂O (0.12 g, 0.432 mmol) at 0° C. The reaction mixture was stirred at room temperature for 1 h. After completion (monitored by TLC), the mixture was concentrated under reduced pressure. The crude was triturated Et₂O and dried under reduced pressure to afford title compound 106 (0.15 g, 80.81%) as an off-white gummy solid. LCMS calculated for C25H30N6O7S: 558.19; Found: 559.30 (M+1).

### Synthesis of methyl (2-(4-ethyl-2-(N-(6-((4-((2-fluoroacrylamido)methyl)-1H-pyrazol-1-yl)methyl)-4-methoxybenzo[d]isoxazol-3-yl)sulfamoyl)phenoxy)ethyl)carbamate (Syn. Ex. 79)

To a stirred solution of 2-fluoroacrylic acid (0.04 g, 0.465 mmol) in DCM (5 mL) were added DIPEA (0.20 mL, 1.163 mmol) and HATU (0.13 g, 0.349 mmol) and compound 106 (0.13 g, 0.232 mmol) at 0° C. The reaction mixture was stirred at room temperature for 1 h. After completion (monitored by TLC), the mixture was quenched with ice-cold water and extracted with 10% MeOH in DCM. The combined organic layer was dried over anhydrous Na₂SO₄ and concentrated under reduced pressure. The crude was purified by prep. HPLC to afford methyl (2-(4-ethyl-2-(N-(6-((4-((2-fluoroacrylamido)methyl)-1H-pyrazol-1-yl)methyl)-4-methoxybenzo[d]isoxazol-3-yl)sulfamoyl)phenoxy)ethyl)carbamate (Syn. Ex. 79; 8 mg, 5.38%) as an off-white solid. (See analytical data in Table 1).

### Example 80

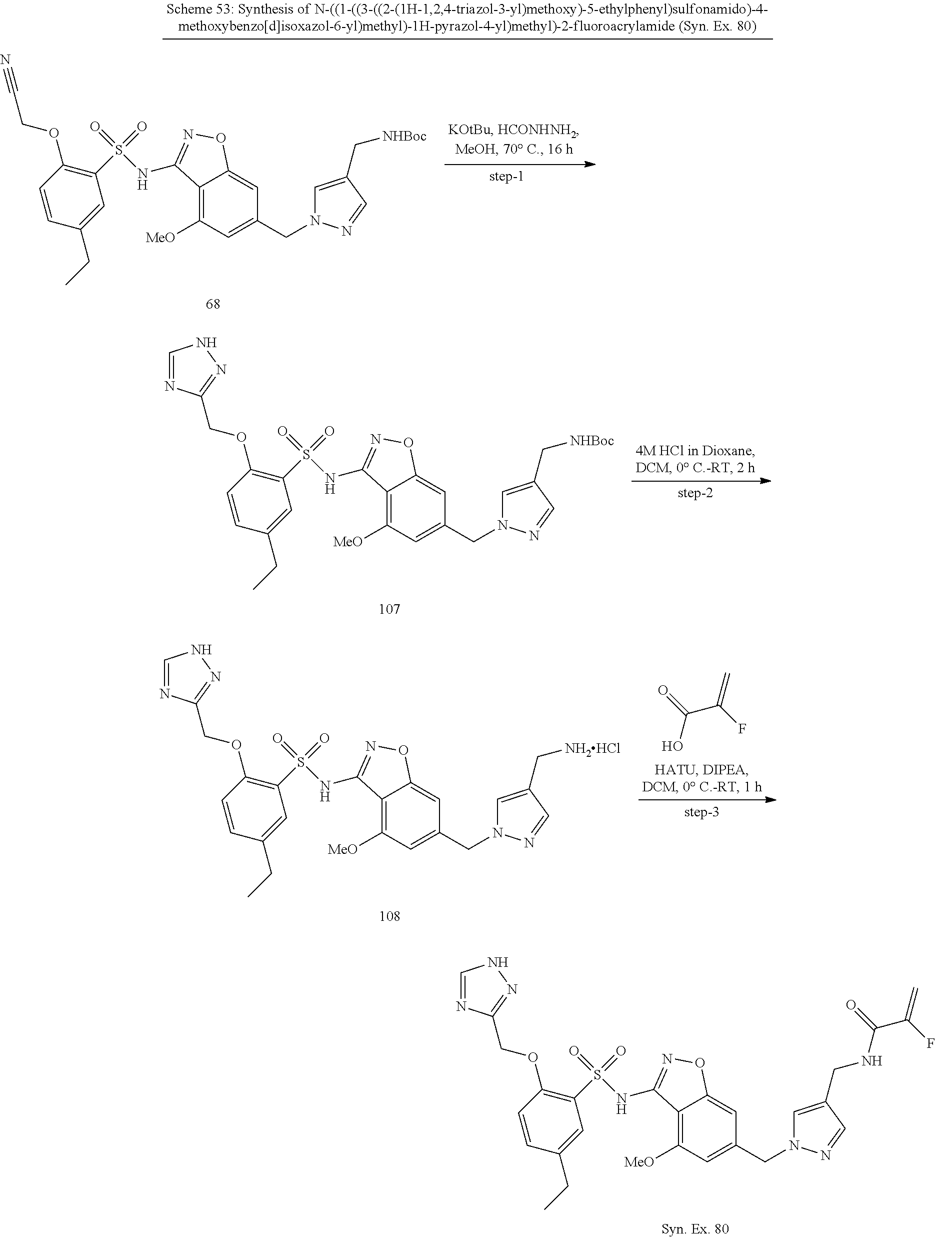

### Synthesis of tert-Butyl ((1-((3-((2-((1H-1,2,4-triazol-3-yl)methoxy)-5-ethylphenyl)sulfonamido)-4-methoxybenzo[d]isoxazol-6-yl)methyl)-1H-pyrazol-4-yl)methyl)carbamate (106)

To a stirred solution of compound 68 (0.20 g, 0.335 mmol) in MeOH (5 mL) was added KOtBu (0.04 g, 0.402 mmol). The reaction mixture was stirred at 70° C. for 2 h and cooled at room temperature followed by addition of HCONHNH₂ (0.10 g, 1.675 mmol). The reaction mixture was stirred at 70° C. for 16 h. After completion (monitored by TLC), the mixture was quenched with ice-cold water and extracted with EtOAc. The combined organic layer was dried over anhydrous Na₂SO₄ and concentrated under reduced pressure. The crude was purified by combi-flash chromatography using 7-11% MeOH/DCM to afford title compound 107 (0.135 g, 59.26%) as a brown semi-solid. LCMS calculated for C29H34N807S: 638.23; Found: 639.20 (M+1).

### Synthesis of 2-((1H-1,2,4-triazol-3-yl)methoxy)-N-(6-((4-(aminomethyl)-1H-pyrazol-1-yl)methyl)-4-methoxybenzo[d]isoxazol-3-yl)-5-ethylbenzenesulfonamide hydrochloride (108)

To a stirred solution of compound 107 (0.13 g, 0.203 mmol) in DCM (3 mL) was added 4M HCl in Dioxane (0.50 mL, 2.035 mmol) at 0° C. The reaction mixture was stirred at room temperature for 2 h. After completion (monitored by TLC), the mixture was concentrated under reduced pressure. The crude was triturated with Et₂O to afford title compound 108 (0.11 g, 93.06%) as a brown solid. LCMS calculated for C24H26N805S: 538.17; Found: 539.20 (M+1).

### Synthesis of N-((1-((3-((2-((1H-1,2,4-triazol-3-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 80)

To a stirred solution of compound 108 (0.10 g, 0.1739 mmol) in DCM (3 mL) were added DIPEA (0.09 mL, 0.521 mmol) and HATU (0.10 g, 0.260 mmol) and 2-fluoroacrylic acid (0.01 g, 0.208 mmol) at 0° C. The reaction mixture was stirred at room temperature for 1 h. After completion (monitored by TLC), the mixture was quenched with ice-cold water, extracted with DCM. The combined organic layer was washed with brine, dried over anhydrous Na₂SO₄, filtered and concentrated under reduced pressure. The crude was purified by prep. HPLC to afford N-((1-((3-((2-((1H-1,2,4-triazol-3-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 80; 26 mg, 23.89%) as an off-white solid. (See analytical data in Table 1).

### Examples 81-82

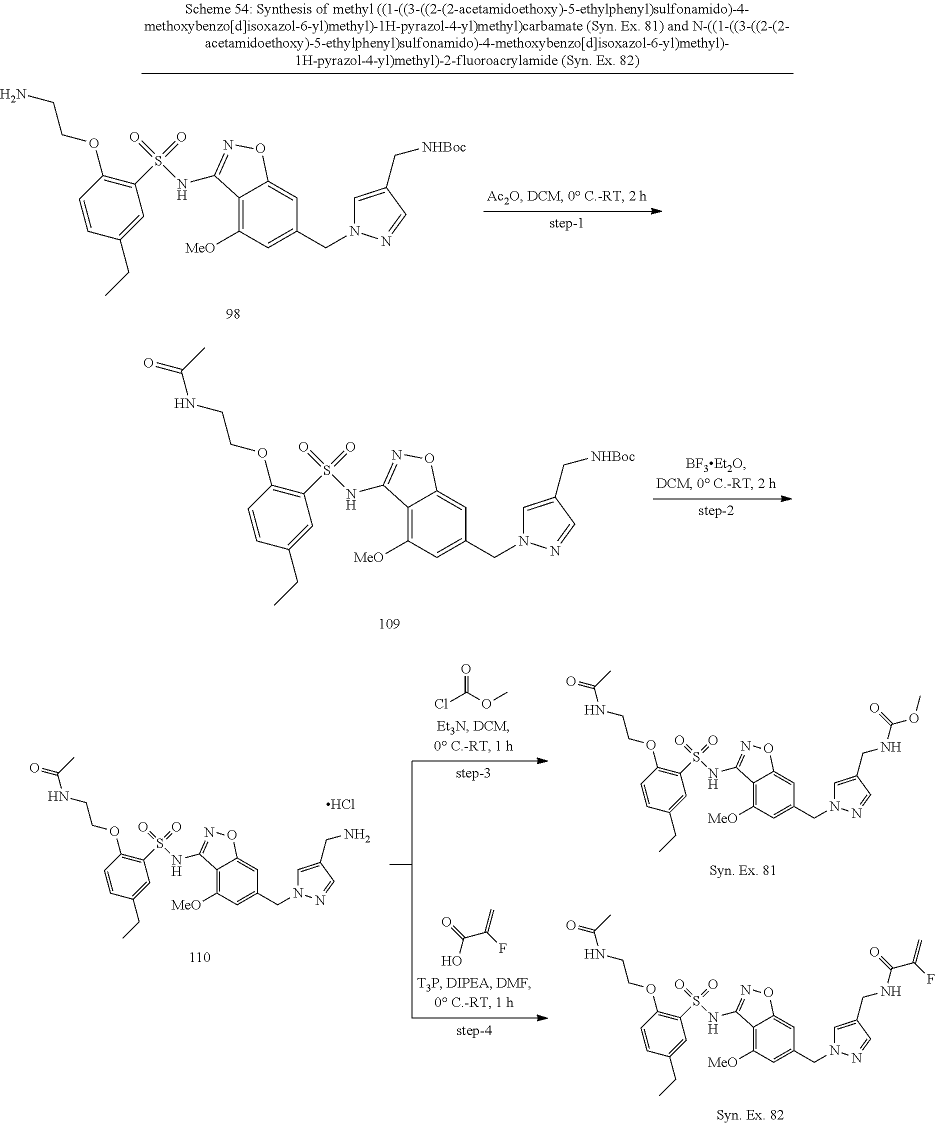

### Synthesis of tert-Butyl ((1-((3-((2-(2-acetamidoethoxy)-5-ethylphenyl)sulfonamido)-4-methoxybenzo[d]isoxazol-6-yl)methyl)-1H-pyrazol-4-yl)methyl)carbamate (109)

To a stirred solution of compound 98 (0.22 g, 0.366 mmol) in DCM (5 mL) was added triethylamine (0.14 mL, 1.098 mmol) and Ac₂O (0.07 mL, 0.732 mmol) at 0° C. The reaction mixture was stirred at room temperature for 2 h. After completion (monitored by TLC), the mixture was quenched with ice-cold water and extracted with 10% MeOH/DCM. The combined organic layer was dried over anhydrous Na₂SO₄ and concentrated under reduced pressure. The crude was triturated with Et₂O to afford title compound 109 (0.18 g, 70.94%) as a pale-yellow solid. LCMS calculated for C30H38N6O8S: 642.25; Found: 643.40 (M+1).

### Synthesis of N-(2-(2-(N-(6-((4-(aminomethyl)-1H-pyrazol-1-yl)methyl)-4-methoxybenzo[d]isoxazol-3-yl)sulfamoyl)-4-ethylphenoxy)ethyl)acetamide (110)

To a stirred solution of compound 109 (0.18 g, 0.280 mmol) in DCM (2 mL) was added BF₃·Et₂O (0.11 g, 0.420 mmol) at 0° C. The reaction mixture was stirred at room temperature for 2 h. After completion (monitored by TLC), the mixture was concentrated under reduced pressure. The crude was triturated with Et₂O and dried under reduced pressure to afford title compound 110 (0.17 g, 99.58%) as a light-yellow solid. LCMS calculated for C₂₅H30N6O6S: 542.19; Found: 543.30 (M+1).

### Synthesis of methyl ((1-((3-((2-(2-acetamidoethoxy)-5-ethylphenyl)sulfonamido)-4-methoxybenzo[d]isoxazol-6-yl)methyl)-1H-pyrazol-4-yl)methyl)carbamate (Syn. Ex. 81)

To a stirred solution of compound 110 (0.08 g, 0.138 mmol) in DCM (5 mL) was added NEt₃ (0.057 mL, 0.414 mmol) and methyl chloroformate (0.026 g, 0.276 mmol) in DCM (0.50 mL) at 0° C. The reaction mixture was stirred at room temperature for 1 h. After completion (monitored by TLC), the mixture was quenched with ice-cold water and extracted with DCM. The combined organic layer was dried over anhydrous Na₂SO₄ and concentrated under reduced pressure. The crude was purified by prep. HPLC to afford methyl ((1-((3-((2-(2-acetamidoethoxy)-5-ethylphenyl)sulfonamido)-4-methoxybenzo[d]isoxazol-6-yl)methyl)-1H-pyrazol-4-yl)methyl)carbamate (Syn. Ex. 81; 18 mg, 21.60%) as an off-white solid. (See analytical data in Table 1).

### Synthesis of N-((1-((3-((2-(2-acetamidoethoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 82)

To a stirred solution of compound 110 (0.10 g, 0.172 mmol) in DMF (2 mL) were added DIPEA (0.09 mL, 0.518 mmol), 2-fluoroacrylic acid 2 (0.03 g, 0.345 mmol) and 50% T₃P in EtOAc (0.32 mL, 0.518 mmol) at 0° C. The reaction mixture was stirred at room temperature for 1 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-((1-((3-((2-(2-acetamidoethoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 82; 18 mg, 16.85%) as an off-white solid. (See analytical data in Table 1).

### Examples 83-84

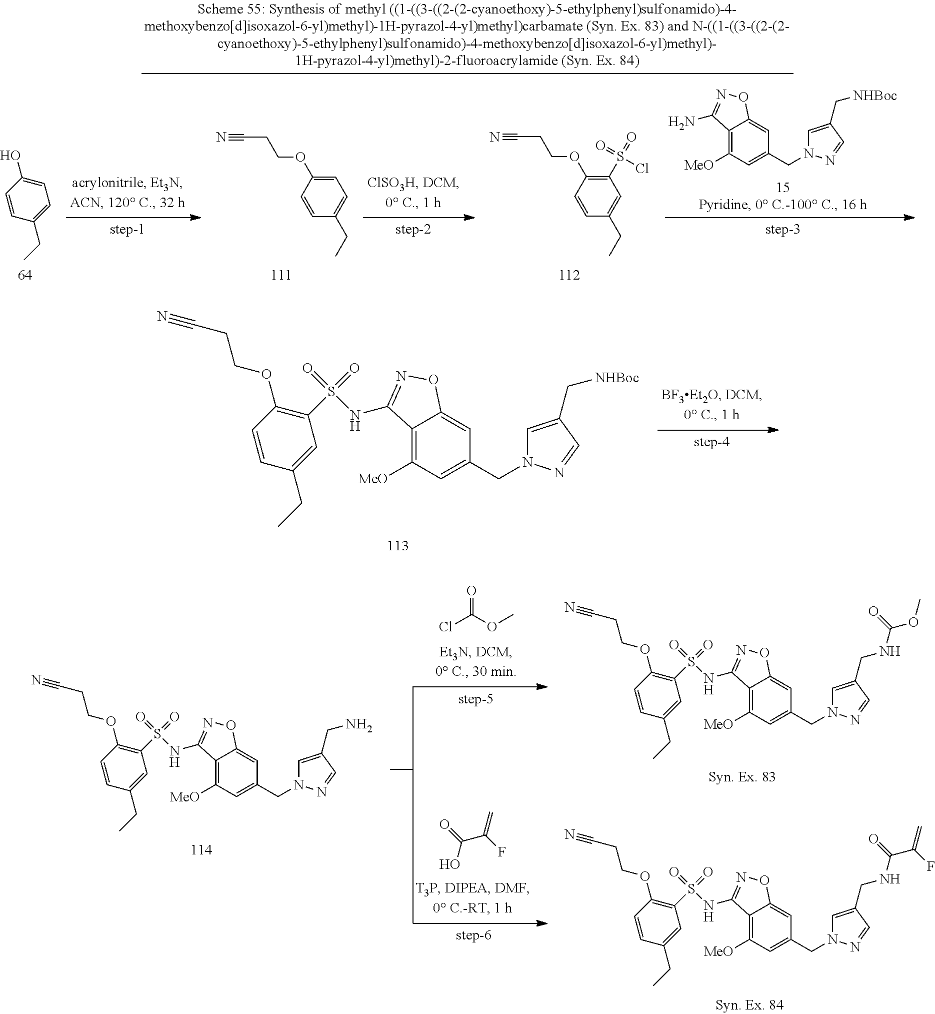

### Synthesis of 3-(4-ethylphenoxy)propanenitrile (111)

To a stirred solution of compound 64 (2 g, 16.370 mmol) in ACN (10 mL) was added Et₃N (27.40 mL, 196.447 mmol) at room temperature. The reaction mixture was stirred for 5 min. followed by the addition of acrylonitrile (10 mL). The reaction mixture was stirred at 120° C. for 32 h. After completion (monitored by TLC), the mixture was quenched with ice-cold water, extracted with EtOAc. The combined organic layer was dried over anhydrous Na₂SO₄, filtered and concentrated under reduced pressure. The crude was diluted with EtOAc and washed with 1M NaOH. The organic layer was dried over anhydrous Na₂SO₄, filtered and concentrated under reduced pressure to afford title compound 111 (1.54 g, 21.49%) as a brown liquid. LCMS calculated for C11H13NO: 175.10; Found: 176.20 (M+1).

### Synthesis of 2-(2-cyanoethoxy)-5-ethylbenzenesulfonyl chloride (112)

To a stirred solution of compound 111 (1.30 g, 7.418 mmol) in DCM (10 mL) was added ClSO₃H (1.49 mL, 22.256 mmol) at 0° C. The reaction mixture was stirred at 0° C. for 1 h. After completion (monitored by TLC), the mixture was quenched ice-cold water and extracted with DCM. The combined organic layer was concentrated under reduced pressure. The crude was purified by combi-flash chromatography using 20-25% EtOAc/Heptane to afford title compound 112 (0.80 g) as a pale-yellow liquid.

### Synthesis of tert-Butyl ((1-((3-((2-(2-cyanoethoxy)-5-ethylphenyl)sulfonamido)-4-methoxybenzo[d]isoxazol-6-yl)methyl)-1H-pyrazol-4-yl)methyl)carbamate (113)

To a stirred solution of compound 15 (0.50 g, 1.339 mmol) in pyridine (5 mL) was added compound 112 (0.54 mL, 2.008 mmol) at 0° C. The reaction mixture was stirred at 100° C. for 16 h. After completion (monitored by TLC), the mixture was concentrated under reduced pressure, co-distilled with toluene and dried under reduced pressure. The crude was purified by combi-flash chromatography using 80-90% EtOAc/Heptane to afford title compound 113 (0.35 g, 38.91%) as an off-white solid. LCMS calculated for C29H34N6O7S: 610.22; Found: 611.30 (M+1).

### Synthesis of N-(6-((4-(aminomethyl)-1H-pyrazol-1-yl)methyl)-4-methoxybenzo[d]isoxazol-3-yl)-2-(2-cyanoethoxy)-5-ethylbenzenesulfonamide (114)

To a stirred solution of compound 113 (0.20 g, 0.327 mmol) in DCM (10 mL) was added BF₃·Et₂O (0.069 g, 0.491 mmol) at 0° C. The reaction mixture was stirred at 0° C. for 1 h. After completion (monitored by TLC), the mixture was concentrated under reduced pressure. The crude was triturated with Et₂O and dried under reduced pressure to afford title compound 114 (0.15 g, 69.28%) as an off-white solid. LCMS calculated for C24H26N6O5S: 510.17; Found: 511.30 (M+1).

### Synthesis of methyl ((1-((3-((2-(2-cyanoethoxy)-5-ethylphenyl)sulfonamido)-4-methoxybenzo[d]isoxazol-6-yl)methyl)-1H-pyrazol-4-yl)methyl)carbamate (Syn. Ex. 83)

To a stirred solution of compound 114 (0.15 g, 0.293 mmol) in DCM (10 mL) were added Et₃N (0.12 mL, 0.881 mmol) and methyl chloroformate (0.033 g, 0.352 mmol) at 0° C. The reaction mixture was stirred at 0° C. for 30 min. After completion (monitored by TLC), the mixture was quenched with ice cold water and extracted with DCM. The combined organic layer was dried over anhydrous Na₂SO₄ and concentrated under reduced pressure. The crude was purified by prep. HPLC to afford methyl ((1-((3-((2-(2-cyanoethoxy)-5-ethylphenyl)sulfonamido)-4-methoxybenzo[d]isoxazol-6-yl)methyl)-1H-pyrazol-4-yl)methyl)carbamate (Syn. Ex. 83; 70 mg, 41.24%) as an off-white solid. (See analytical data in Table 1).

### Synthesis of N-((1-((3-((2-(2-cyanoethoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 84)

To a stirred solution of compound 114 (0.08 g, 0.156 mmol) in DMF (4 mL) were added DIPEA (0.08 g, 0.470 mmol), 2-fluoroacrylic acid (0.029 g, 0.313 mmol) and T₃P 50% in EtOAc (0.29 g, 0.470 mmol) at 0° C. The reaction mixture was stirred at room temperature for 1 h. After completion (monitored by TLC), mixture was quenched with ice-cold water and extracted with EtOAc. The combined organic layer was dried over anhydrous Na₂SO₄ and concentrated under reduced pressure. The crude was purified by prep. HPLC to afford N-((1-((3-((2-(2-cyanoethoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 84; 70 mg, 69.86%) as an off-white solid. (See analytical data in Table 1).

### Examples 85-87

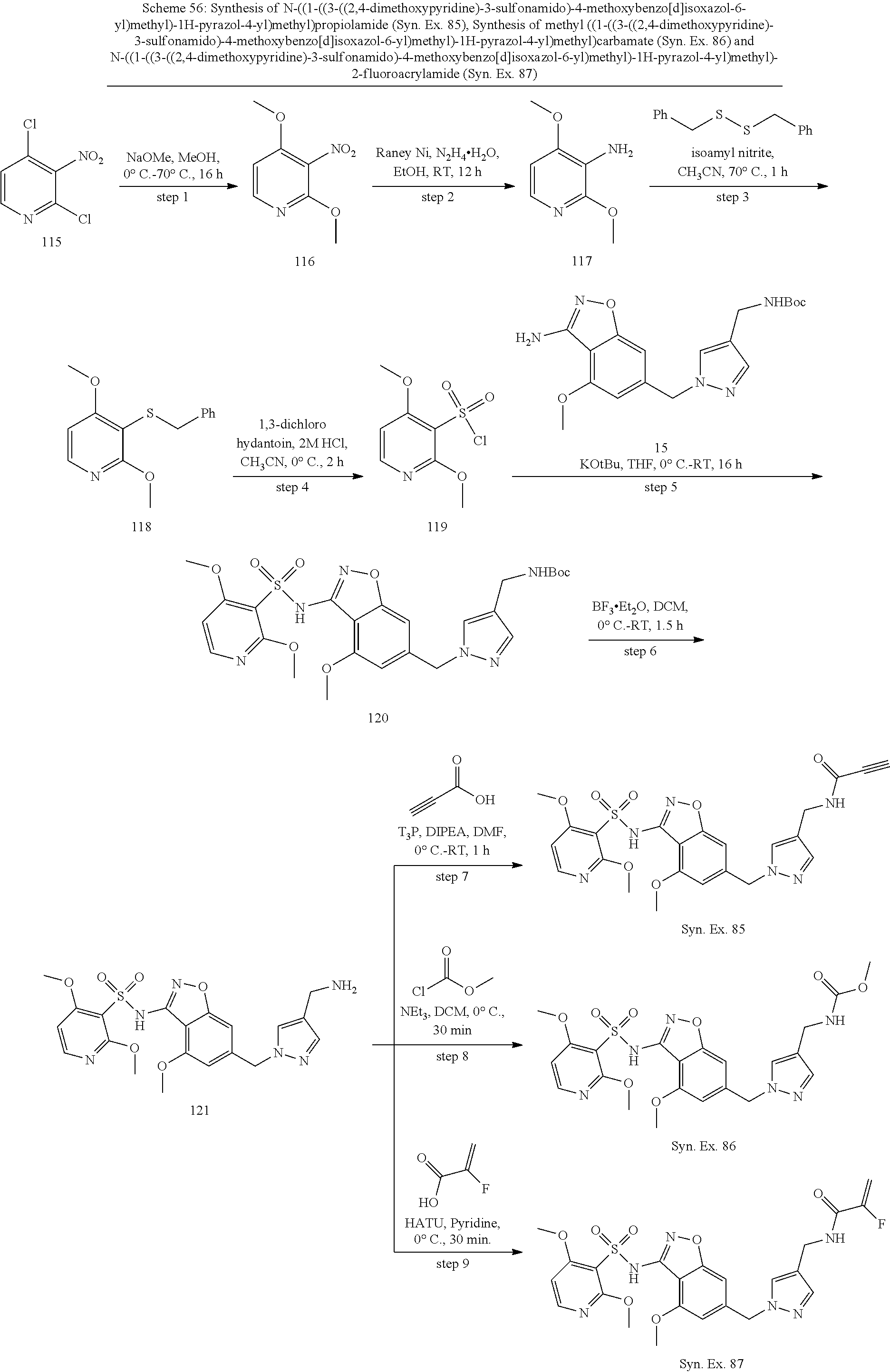

### Synthesis of 2,4-dimethoxy-3-nitropyridine (116)

To a stirred solution of compound 115 (5 g, 25.909 mmol) in MeOH (50 mL) was added NaOMe (7.14 g, 129.547 mmol) at 0° C. The reaction mixture was stirred at 70° C. for 16 h. After completion (monitored by TLC), the mixture was concentrated under reduced pressure, quenched with ice cold water and extracted with EtOAc. The organic layer was dried over anhydrous Na₂SO₄, filtered, and concentrated under reduced pressure to afford title compound 116 (4.70 g, 64.03%) as an off-white solid. LCMS calculated for C7H8N2O4: 184.05; Found: 185.10 (M+1).

### Synthesis of 2,4-dimethoxypyridin-3-amine (117)

To a stirred solution of compound 116 (4.80 g, 26.065 mmol) in EtOH (40 mL) was added Raney Nickel (0.78 g, 9.122 mmol) and 1M hydrazine solution in THF (5.99 mL, 5.99 mmol) at 0° C. The reaction mixture was stirred at room temperature for 12 h. After completion (monitored by TLC), the mixture was filtered through a celite pad and filtrate was concentrated under reduced pressure to afford title compound 117 (3.90 g, 96.08%) as an off-white solid. LCMS calculated for C7H10N2O2: 154.07; Found: 155.10 (M+1).

### Synthesis of 3-(benzylthio)-2,4-dimethoxypyridine (118)

To a stirred solution of compound 117 (3 g, 19.459 mmol) in ACN (30 mL) was added 1,2-dibenzyldisulfane (5.60 g, 22.76 mmol) followed by the addition of isoamyl nitrite (6.30 mL). The resulting mixture was stirred at 70° C. for 1 h. After completion (monitored by TLC), the mixture was quenched with ice-cold water and extracted with EtOAc. The combined organic layer was washed with brine, dried over anhydrous Na₂SO₄ and concentrated under reduced pressure. The crude was purified by combi-flash chromatography using 30% EtOAc/Heptane to afford title compound 118 (1.40 g, 26.70%) as a light brown liquid. LCMS calculated for C14H15NO2S: 261.08; Found: 262.10 (M+1).

### Synthesis of 2,4-dimethoxypyridine-3-sulfonyl chloride (119)

To a stirred solution of compound 118 (1.30 g, 4.974 mmol) in ACN (52 mL), H₂O (1.30 mL) and AcOH (2.60 mL) was added 1,3-dichloro-5,5-dimethylhydantoin (2 g, 9.948 mmol) at 0° C. The resulting mixture was stirred at 0° C. for 2 h. After completion (monitored by TLC), the mixture was quenched with ice-cold water and extracted with EtOAc. The combined organic layer was washed with saturated NaHCO₃, dried over anhydrous Na₂SO₄, filtered and concentrated under reduced pressure to afford title compound 119 (1 g, 84.59%) as a brown solid. LCMS calculated for C7H8ClNO4S: 236.99; Found: 238.0 (M+1).

### Synthesis of tert-Butyl ((1-((3-((2,4-dimethoxypyridine)-3-sulfonamido)-4-methoxybenzo[d]isoxazol-6-yl)methyl)-1H-pyrazol-4-yl)methyl)carbamate (120)

To a stirred solution of compound 15 (0.40 g, 1.071 mmol) in Dry THF (10 mL) was added 1M KOtBu in THF (3.21 g, 3.213 mmol) at 0° C. The resulting mixture was stirred at 0° C. for 10 min. followed by the addition of compound 119 (0.38 g, 1.606 mmol). The reaction mixture was stirred at room temperature for 16 h. After completion (monitored by TLC), the mixture was quenched with ice cold water, extracted with EtOAc. The aqueous layer was acidified with 1N HCl to adjust the pH˜6 and extracted with EtOAc. The combined organic layer was washed with brine, dried over anhydrous Na₂SO₄, filtered and concentrated under reduced pressure to afford title compound 120 (0.30 g, 46.30%) as a brown solid. LCMS calculated for C25H30N6O8S: 574.18, Found: 519.10 [(M-tBu)+1].

### Synthesis of N-(6-((4-(aminomethyl)-1H-pyrazol-1-yl)methyl)-4-methoxybenzo[d]isoxazol-3-yl)-2,4-dimethoxypyridine-3-sulfonamide (121)

To a stirred solution of compound 120 (0.25 g, 0.435 mmol) in DCM (10 mL) was added BF₃·Et₂O (0.24 g, 0.870 mmol) at 0° C. The reaction mixture was stirred at room temperature for 1.5 h. After completion (monitored by TLC), the mixture was concentrated under reduced pressure. The crude was triturated Et₂O and dried under reduced pressure to afford title compound 121 (0.20 g, 93.02%) as an off-white solid. LCMS calculated for C₂₀H22N6O6S: 474.13, Found: 473.20 (M−1).

### Synthesis of N-((1-((3-((2,4-dimethoxypyridine)-3-sulfonamido)-4-methoxybenzo[d]isoxazol-6-yl)methyl)-1H-pyrazol-4-yl)methyl)propiolamide (Syn. Ex. 85)

To a stirred solution of compound 121 (0.10 g, 0.210 mmol) in DMF (3 mL) was added DIPEA (0.11 mL, 0.632 mmol), propiolic acid (0.029 g, 0.421 mmol) and 50% T₃P in EtOAc (0.40 mL, 0.632 mmol) at 0° C. The reaction mixture was stirred at room temperature for 1 h. After completion (monitored by TLC), the mixture was quenched with ice-cold water and extracted with EtOAc. The combined organic layer was dried over anhydrous Na₂SO₄ filtered and concentrated under reduced pressure. The crude was purified by prep. HPLC to afford N-((1-((3-((2,4-dimethoxypyridine)-3-sulfonamido)-4-methoxybenzo[d]isoxazol-6-yl)methyl)-1H-pyrazol-4-yl)methyl)propiolamide (Syn. Ex. 85; 4.00 mg, 3.46%) as an off-white solid. (See analytical data in Table 1).

### Synthesis of methyl ((1-((3-((2,4-dimethoxypyridine)-3-sulfonamido)-4-methoxybenzo[d]isoxazol-6-yl)methyl)-1H-pyrazol-4-yl)methyl)carbamate (Syn. Ex. 86)

To a stirred solution of compound 121 (0.10 g, 0.210 mmol) in DCM (10 mL) was added methyl chloroformate (0.019 g, 0.210 mmol) and NEt₃ (0.058 mL, 0.421 mmol) at 0° C. The resulting mixture was stirred at 0° C. for 30 min. After completion (monitored by TLC), the mixture was quenched with ice-cold water and extracted with DCM. The combined organic layer was dried over anhydrous Na₂SO₄ filtered and concentrated under reduced pressure. The crude was purified by prep. HPLC to afford methyl ((1-((3-((2,4-dimethoxypyridine)-3-sulfonamido)-4-methoxybenzo[d]isoxazol-6-yl)methyl)-1H-pyrazol-4-yl)methyl)carbamate (Syn. Ex. 86; 18 mg, 16.04%) as an off-white solid. (See analytical data in Table 1).

### Synthesis of N-((1-((3-((2,4-dimethoxypyridine)-3-sulfonamido)-4-methoxybenzo[d]isoxazol-6-yl)methyl)-1H-pyrazol-4-yl)methyl)-2-fluoroacrylamide (Syn. Ex. 87)

To a stirred solution of compound 121 (0.10 g, 0.210 mmol) in pyridine (5 mL) were added 2-fluoroacrylic acid (0.02 g, 0.252 mmol) and HATU (0.16 g, 0.421 mmol) at 0° C. The reaction mixture was stirred at 0° C. for 30 min. After completion (monitored by TLC), the mixture was quenched with water and extracted with EtOAc. The combined organic layer was dried over anhydrous Na₂SO₄, filtered and concentrated under reduced pressure. The crude was purified prep. HPLC to afford N-((1-((3-((2,4-dimethoxypyridine)-3-sulfonamido)-4-methoxybenzo[d]isoxazol-6-yl)methyl)-1H-pyrazol-4-yl)methyl)-2-fluoroacrylamide (Syn. Ex. 87; 9 mg, 7.78%) as an off-white solid. (See analytical data in Table 1).

### Examples 88-89

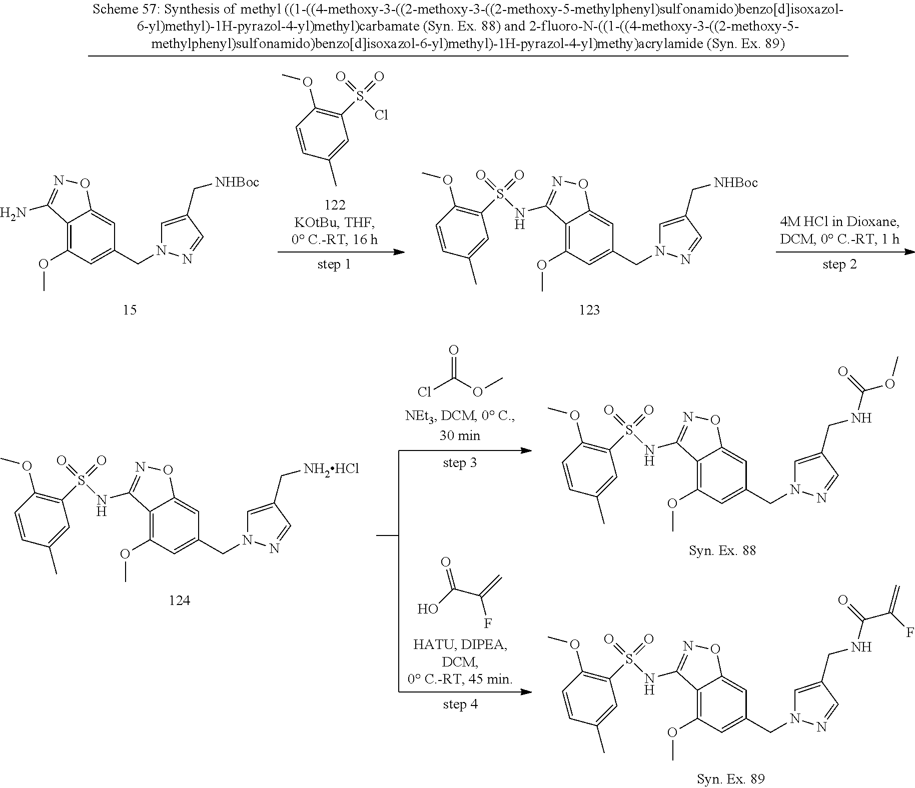

### Synthesis of tert-Butyl ((1-((4-methoxy-3-((2-methoxy-5-methylphenyl)sulfonamido)benzo[d]isoxazol-6-yl)methyl)-1H-pyrazol-4-yl)methyl)carbamate (123)

To a stirred solution of compound 15 (0.50 g, 1.339 mmol) in THF (15 mL) was added 1M KOtBu in THF (1 mL, 4.017 mmol) at 0° C. The reaction mixture was stirred at 0° C. for 10 min followed by the addition of compound 122 (0.44 g, 2.008 mmol). The reaction mixture was stirred at room temperature for 16 h. After completion (monitored by TLC), the mixture was quenched with ice-cold water and extracted with EtOAc. The organic layer was dried over anhydrous Na₂SO₄, filtered and concentrated under reduced pressure. The crude was purified by combi-flash chromatography using 90-92% EtOAc/Heptane to afford title compound 123 (0.30 g, 34.55%) as an off-white solid. LCMS calculated for C26H31N5O7S: 557.19; Found: 556.30 (M−1).

### Synthesis of N-(6-((4-(aminomethyl)-1H-pyrazol-1-yl)methyl)-4-methoxybenzo[d]isoxazol-3-yl)-2-methoxy-5-methylbenzenesulfonamide hydrochloride (124)

To a stirred solution of compound 123 (0.30 g, 0.538 mmol) in DCM (3 mL) was added 4M HCl in Dioxane (3 mL) at 0° C. The reaction mixture was stirred at room temperature for 1 h. After completion (monitored by TLC), the mixture was concentrated under reduced pressure. The crude was triturated with Et₂O and dried under reduced pressure to afford title compound 124 (0.25 g, 69.61%) as an off-white solid. LCMS calculated for C21H23N5O5S: 457.14; Found: 456.10 (M−1).

### Synthesis of methyl ((1-((4-methoxy-3-((2-methoxy-5-methylphenyl)sulfonamido)benzo[d]isoxazol-6-yl)methyl)-1H-pyrazol-4-yl)methyl)carbamate (Syn. Ex. 88)

To a stirred solution of compound 124 (0.12 g, 0.243 mmol) in DCM (5 mL) were added NEt₃ (0.16 mL, 1.216 mmol) and methyl chloroformate (0.027 g, 0.292 mmol) at 0° C. The reaction mixture was stirred at 0° C. for 30 min. After completion (monitored by TLC), the mixture was quenched with water and extracted with DCM. The organic layer was dried over anhydrous Na₂SO₄, filtered and concentrated under reduced pressure. The crude was purified by prep. HPLC to afford methyl ((1-((4-methoxy-3-((2-methoxy-5-methylphenyl)sulfonamido)benzol[d]isoxazol-6-yl)methyl)-1H-pyrazol-4-yl)methyl)carbamate (Syn. Ex. 88; 24 mg, 18.79%) as an off-white solid. (See analytical data in Table 1).

### Synthesis of 2-fluoro-N-((1-((4-methoxy-3-((2-methoxy-5-methylphenyl)sulfonamido)benzo[d]isoxazol-6-yl)methyl)-1H-pyrazol-4-yl)methyl)acrylamide (Syn. Ex. 89)

To a stirred solution of compound 124 (0.10 g, 0.202 mmol) in DCM (2 mL) were added and DIPEA (0.17 mL, 1.012 mmol), 2-fluoroacrylic acid (0.036 g, 0.404 mmol) and HATU (0.12 g, 0.303 mmol) at 0° C. The reaction mixture was stirred at room temperature for 45 min. After completion (monitored by TLC), the mixture was quenched with water and extracted with EtOAc. The combined organic layer was dried over anhydrous Na₂SO₄ and concentrated under reduced pressure. The crude was purified by prep. HPLC to afford 2-fluoro-N-((1-((4-methoxy-3-((2-methoxy-5-methylphenyl)sulfonamido)benzo[d]isoxazol-6-yl)methyl)-1H-pyrazol-4-yl)methyl)acrylamide (Syn. Ex. 89; 33 mg, 30.49%) as a yellow solid. (See analytical data in Table 1).

### Examples 90-91

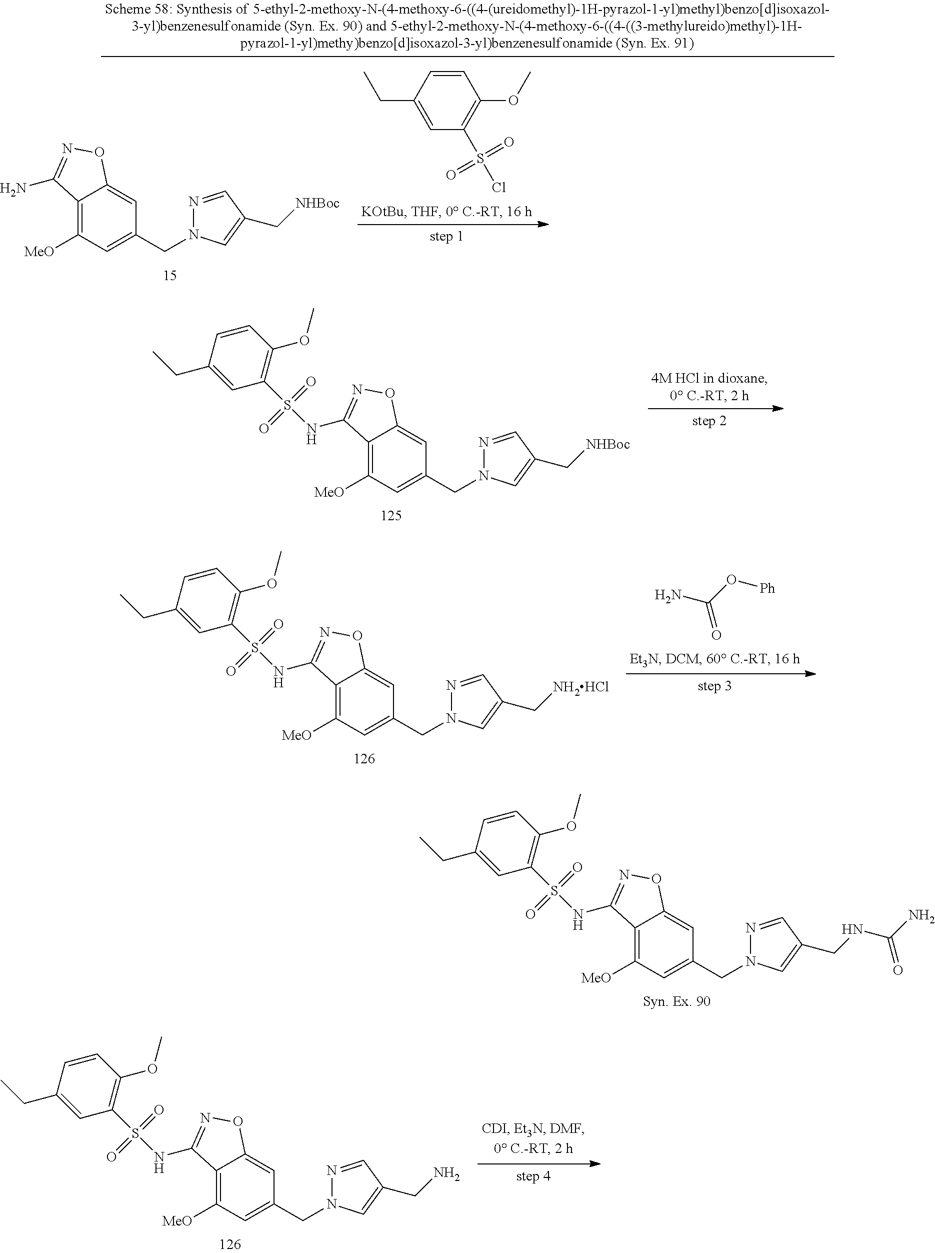

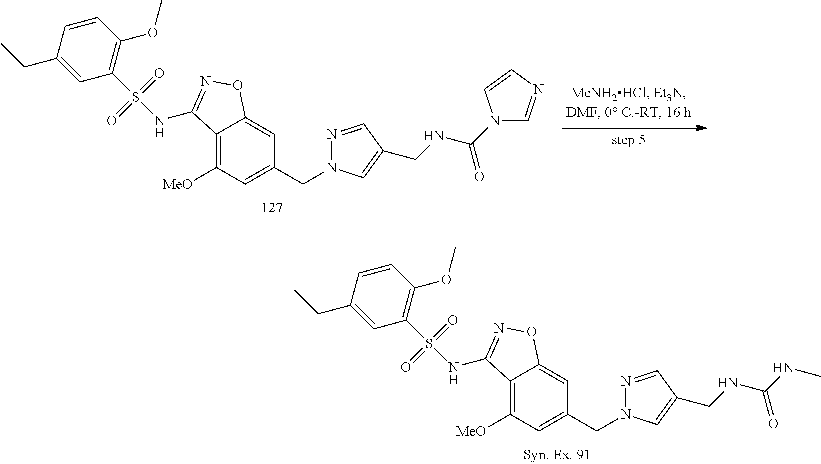 [SMILES: CCc1ccc(OC)c(S(=O)(=O)Nc2noc3cc(Cn4cc(CNC(=O)n5ccnc5)cn4)cc(OC)c23)c1 ; CCc1ccc(OC)c(S(=O)(=O)Nc2noc3cc(Cn4cc(CNC(=O)NC)cn4)cc(OC)c23)c1]

### Synthesis of tert-Butyl ((1-((3-((5-ethyl-2-methoxyphenyl)sulfonamido)-4-methoxybenzo[d]isoxazol-6-yl)methyl)-1H-pyrazol-4-yl)methyl)carbamate (125)

To a stirred solution of compound 15 (2 g, 5.356 mmol) in THF (40 mL) was added 1M KOtBu in THF (16.06 mL, 16.068 mmol) at 0° C. The reaction was stirred for 10 min followed by addition of 5-ethyl-2-methoxybenzenesulfonyl chloride (1.88 g, 8.034 mmol) in THF (6 mL) at 0° C. The reaction mixture was stirred at room temperature for 16 h. After completion (monitored by TLC), the mixture was quenched with ice-cold water and extracted with EtOAc. The organic layer was dried over anhydrous Na₂SO₄, filtered and concentrated under reduced pressure. The crude was purified by combi-flash chromatography using 70-72% EtOAc/Heptane to afford title compound 125 (1.20 g, 31.35%) as an off-white solid. LCMS calculated for C27H33N5O7S: 571.21; Found: 572.68 (M+1).

### Synthesis of N-(6-((4-(aminomethyl)-1H-pyrazol-1-yl)methyl)-4-methoxybenzo[d]isoxazol-3-yl)-5-ethyl-2-methoxybenzenesulfonamide hydrochloride (126)

To a stirred solution of compound 125 (0.35 g, 0.528 mmol) in DCM (6 mL) was added 4M HCl in 1,4-Dioxane (1.53 mL, 6.122 mmol) at 0° C. The reaction mixture was stirred at room temperature for 2 h. After completion (monitored by TLC), the mixture was concentrated under reduced pressure. The crude was triturated with Et₂O and dried under reduced pressure to afford title compound 126 (0.29 g, 82.99%) as an off-white solid. LCMS calculated for C22H25N5O5S S: 471.16; Found: 470.20 (M−1).

### Synthesis of 5-ethyl-2-methoxy-N-(4-methoxy-6-((4-(ureidomethyl)-1H-pyrazol-1-yl)methyl)benzo[d]isoxazol-3-yl)benzenesulfonamide (Syn. Ex. 90)

To a stirred solution of compound 126 (0.10 g, 0.196 mmol) in THF (3 mL) was added phenyl carbamate (32 mg, 0.236 mmol) and NEt₃ (0.08 mL, 0.590 mmol) at room temperature. The reaction mixture was stirred at 60° C. for 16 h. After completion (monitored by TLC), the mixture was quenched with ice-cold water, acidified with saturated citric acid and extracted with EtOAc. The combined organic layer was washed with brine, dried over anhydrous Na₂SO₄, filtered and concentrated under reduced pressure. The crude was purified by prep. HPLC to afford 5-ethyl-2-methoxy-N-(4-methoxy-6-((4-(ureidomethyl)-1H-pyrazol-1-yl)methyl)benzo[d]isoxazol-3-yl)benzenesulfonamide (Syn. Ex. 90; 21 mg, 19.92%) as an off-white solid. (See analytical data in Table 1).

### Synthesis of N-((1-((3-((5-ethyl-2-methoxyphenyl)sulfonamido)-4-methoxybenzo[d]isoxazol-6-yl)methyl)-1H-pyrazol-4-yl)methyl)-1H-imidazole-1-carboxamide (127)

To a stirred solution of compound 126 (0.13 g, 0.255 mmol) in DMF (2 mL) was added Et₃N (0.07 mL, 0.511 mmol) and CDI (0.06 g, 0.383 mmol) at 0° C. The resulting mixture was stirred at room temperature for 2 h. After completion (monitored by TLC), the mixture was quenched with ice-cold water, acidified with saturated citric acid and extracted with EtOAc. The combined organic layer was washed with brine, dried over anhydrous Na₂SO₄, filtered and concentrated under reduced pressure. The crude was purified by combi-flash chromatography using 7-12% of MeOH/DCM to afford title compound 127 (0.11 g, 69.84%) as a brown gummy solid. LCMS calculated for C26H27N706S: 565.17; Found: 566.30 (M+1).

### Synthesis of 5-ethyl-2-methoxy-N-(4-methoxy-6-((4-((3-methylureido)methyl)-1H-pyrazol-1-yl)methyl)benzo[d]isoxazol-3-yl)benzenesulfonamide (Syn. Ex. 91)

To a stirred solution of compound 127 (0.10 g, 0.176 mmol) in DMF (2 mL) were added Et₃N (0.07 mL, 0.530 mmol) followed by the addition of MeNH₂·HCl (0.01 g, 0.212 mmol) at 0° C. The resulting mixture was stirred at room temperature for 16 h. After completion (monitored by TLC), the mixture was quenched with ice-cold water, acidified with saturated citric acid and extracted with EtOAc. The combined organic layer was washed with brine, dried over anhydrous Na₂SO₄, filtered and concentrated under reduced pressure. The crude was purified by prep. HPLC to afford 5-ethyl-2-methoxy-N-(4-methoxy-6-((4-((3-methylureido)methyl)-1H-pyrazol-1-yl)methyl)benzol[d]isoxazol-3-yl)benzenesulfonamide (Syn. Ex. 91; 5.00 mg, 5.29%) as an off-white solid. (See analytical data in Table 1).

### Examples 92-93

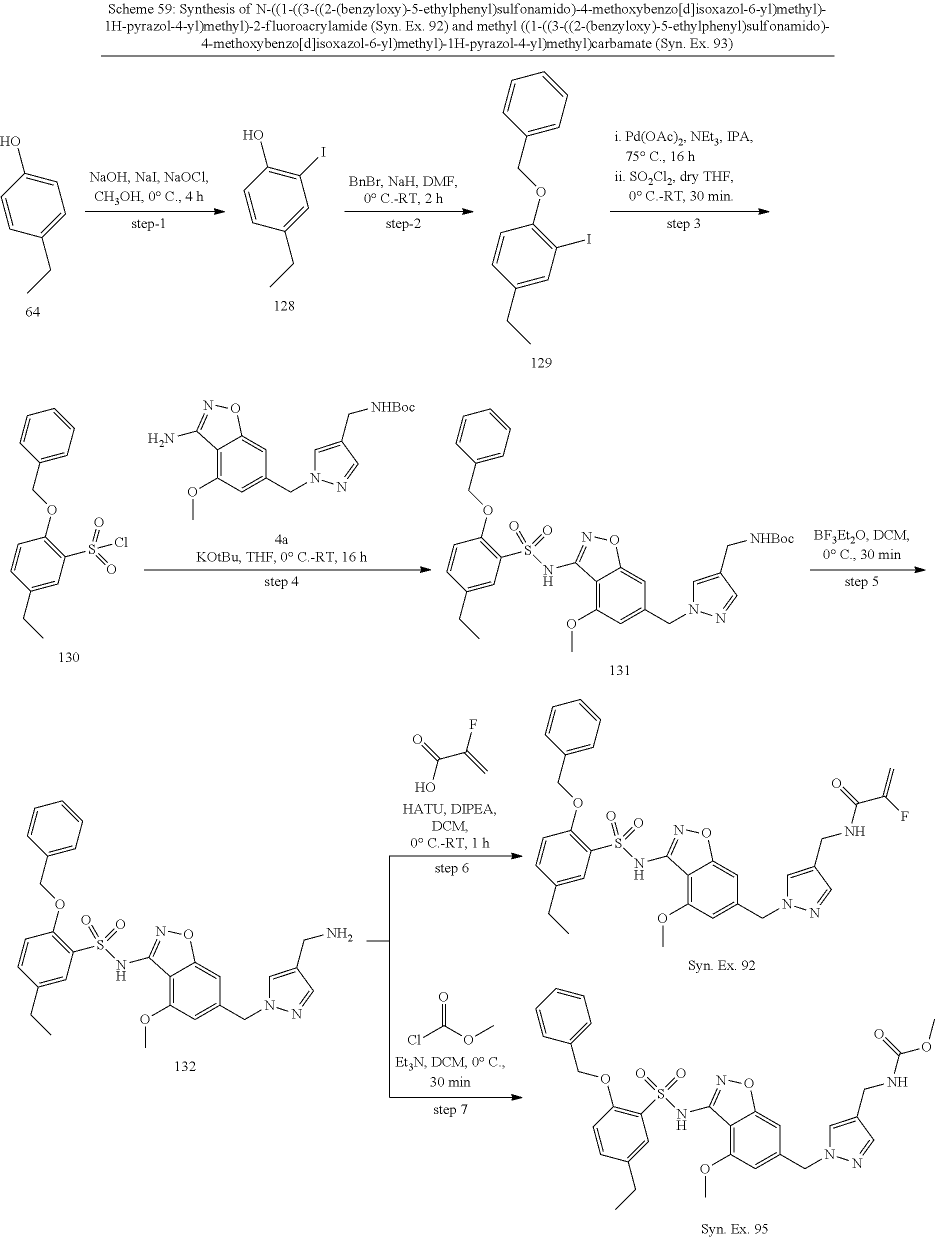

### Synthesis of 4-ethyl-2-iodophenol (128)

To a solution of compound 64 (5 g, 40.926 mmol) in MeOH (50 mL) were added NaI (18.58 g, 122.779 mmol) and NaOH (3.91 g, 94.949 mmol) followed by NaOCl (50 mL) at 0° C. The reaction mixture was stirred at 0° C. for 4 h. After completion (monitored by TLC), the mixture was quenched with 10% Na₂S2O₃, acidified with 2N HCl to pH ˜6-5 and extracted with EtOAc. The combined organic layer was dried over anhydrous Na₂SO₄, filtered and concentrated under reduced pressure. The crude was purified by combi-flash chromatography using 0-5% EtOAc/Heptane to afford title compound 128 (8.90 g, 71%) as a pale-yellow liquid. LCMS calculated for C8H9IO: 247.97; Found: 247 (M−1).

### Synthesis of 1-(benzyloxy)-4-ethyl-2-iodobenzene (129)

To a stirred solution of compound 128 (4 g, 16.125 mmol) in DMF (35 mL) were added 60% NaH in oil (1.28 g, 32.250 mmol) and benzyl bromide (3.30 g, 19.350 mmol) at 0° C. The reaction mixture was stirred at room temperature for 2 h. After completion (monitored by TLC), the mixture was quenched with ice-cold water and extracted with EtOAc. The combined organic layer was washed with brine, dried over anhydrous Na₂SO₄, filtered and concentrated under reduced pressure. The crude was purified by combi-flash chromatography using 4% EtOAc/Heptane to afford title compound 129 (3.0 g, 37.40%) as a brown oil. LCMS calculated for C15H15IO: 338.02; Found: 339.30 (M+1).

### Synthesis of 2-(benzyloxy)-5-ethylbenzenesulfonyl chloride (130)

To a stirred solution of 1-(benzyloxy)-4-ethyl-2-iodobenzene 129 (0.32 g, 0.946 mmol) in IPA (6 mL) were added NEt₃ (0.39 mL, 2.838 mmol), di(1-adamantyl)-n-butylphosphine (0.02 g, 0.075 mmol) and 1,4-diazabicyclo[2.2.2]octane-1,4-diium-1,4-disulfinate (0.13 g, 0.567 mmol) under Argon atmosphere. The reaction mixture was degassed for 5 min followed by addition of Pd(OAc)₂ (0.01 g, 0.047 mmol). The reaction mixture was stirred at 75° C. for 16 h. After completion (monitored by TLC), the mixture was cooled to room temperature and filtered through a celite pad. The filtrate was concentrated under reduced pressure and crude was dissolved in dry THF (6 mL) and SO₂Cl₂ (0.23 mL, 2.839 mmol) was added at 0° C. The reaction mixture was stirred at room temperature for 30 min. After completion (monitored by TLC), the mixture was quenched with NH₄Cl, extracted with DCM and concentrated under reduced pressure to afford 2-(benzyloxy)-5-ethylbenzenesulfonyl chloride 130 (0.28 g, 95.23%). The crude was carried forward to the next reaction.

### Synthesis of tert-Butyl ((1-((3-((2-(benzyloxy)-5-ethylphenyl)sulfonamido)-4-methoxybenzo[d]isoxazol-6-yl)methyl)-1H-pyrazol-4-yl)methyl)carbamate (131)

To a stirred solution of compound 15 (0.45 g, 1.205 mmol) in THF (6 mL) was added 1M KOtBu in THF (3.61 mL, 3.615 mmol) at 0° C. The reaction was stirred for 10 min followed by addition of compound 130 (0.44 g, 1.446 mmol) at 0° C. The reaction mixture was stirred at room temperature for 16 h. After completion (monitored by TLC), the mixture was quenched with water and extracted with EtOAc. The organic layer was dried over anhydrous Na₂SO₄, filtered and concentrated under reduced pressure. The crude was purified by combi-flash chromatography using 68-78% EtOAc/Heptane to afford title compound 131 (0.40 g, 48.88%) as a brown solid. LCMS calculated for C33H37N5O7S: 647.24; Found: 646.30 (M−1).

### Synthesis of N-(6-((4-(aminomethyl)-1H-pyrazol-1-yl)methyl)-4-methoxybenzo[d]isoxazol-3-yl)-2-(benzyloxy)-5-ethylbenzenesulfonamide (132)

To a stirred solution of compound 131 (0.30 g, 0.463 mmol) in DCM (6 mL) was added BF₃·Et₂O (0.17 mL, 0.694 mmol) at 0° C. The reaction mixture was stirred at 0° C. for 30 min. After completion (monitored by TLC), the mixture was concentrated under reduced pressure. The crude was triturated with Et₂O and concentrated under reduced pressure to afford title compound 132 (0.22 g, 79.10%) as a brown solid. LCMS calculated for C28H29N5O5S: 547.19; Found: 546.30 (M−1).

### Synthesis of N-((1-((3-((2-(benzyloxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 92)

To a stirred solution of compound 132 (0.11 g, 0.20 mmol) in DCM (4 mL) were added 2-fluoroacrylic acid (0.02 g, 0.241 mmol), DIPEA (0.10 mL, 0.602 mmol) and HATU (0.11 g, 0.301 mmol) at 0° C. The reaction mixture was stirred at room temperature for 1 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-((1-((3-((2-(benzyloxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 92; 16 mg, 12.81%) as an off-white solid. (See analytical data in Table 1).

### Synthesis of methyl ((1-((3-((2-(benzyloxy)-5-ethylphenyl)sulfonamido)-4-methoxybenzo[d]isoxazol-6-yl)methyl)-1H-pyrazol-4-yl)methyl)carbamate (Syn. Ex. 93)

To a stirred solution of compound 132 (0.10 g, 0.182 mmol) in DCM (3 mL) were added NEt₃ (0.07 mL, 0.547 mmol) and methyl chloroformate (0.02 g, 0.219 mmol) at 0° C. The reaction mixture was stirred at 0° C. for 30 min. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford methyl ((1-((3-((2-(benzyloxy)-5-ethylphenyl)sulfonamido)-4-methoxybenzo[d]isoxazol-6-yl)methyl)-1H-pyrazol-4-yl)methyl)carbamate (Syn. Ex. 93; 48 mg, 42.32%) as an off-white solid. (See analytical data in Table 1).

### Examples 94-95

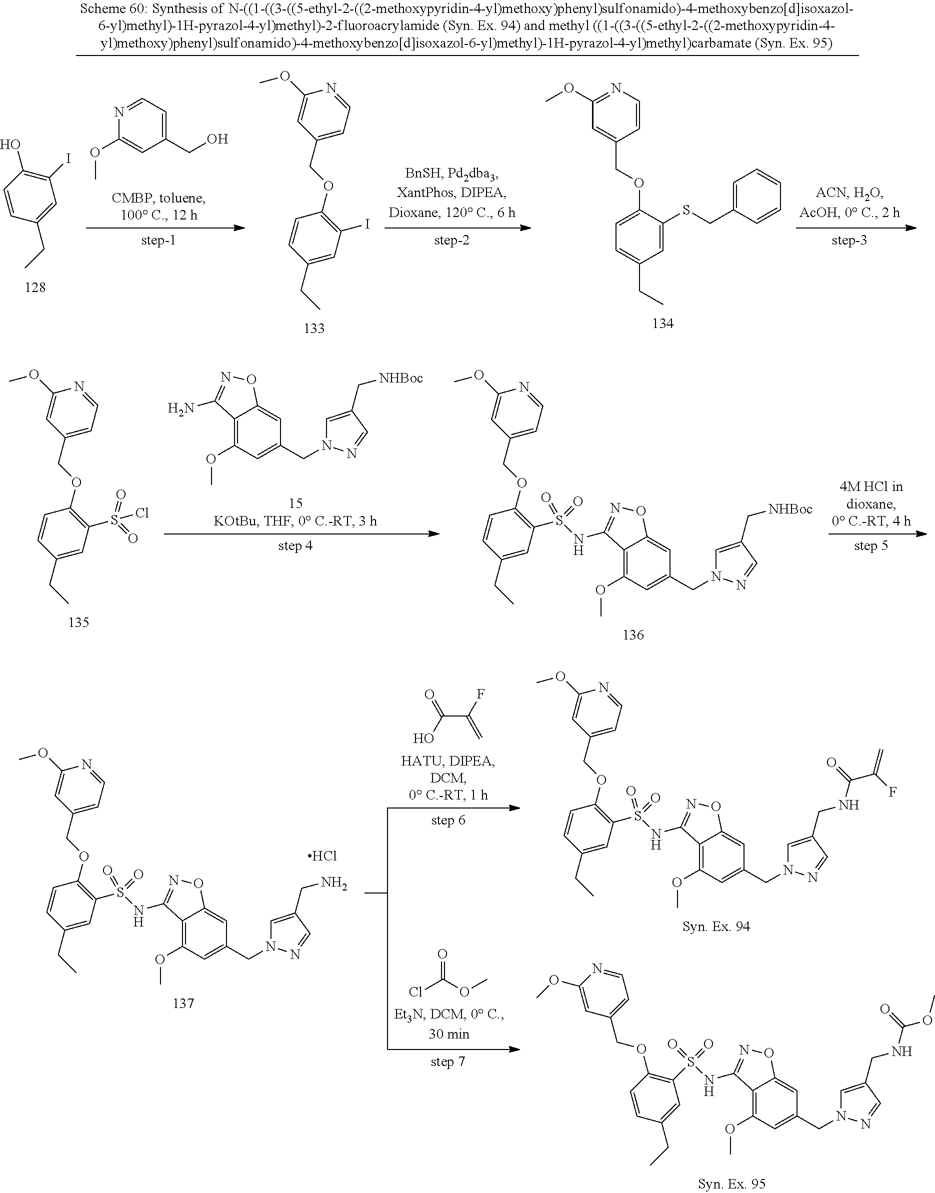

### Synthesis of 4-((4-ethyl-2-iodophenoxy)methyl)-2-methoxypyridine (133)

To a stirred solution of compound 128 (10 g, 40.312 mmol) in toluene (100 mL) were added (2-methoxypyridin-4-yl)methanol (6.73 g, 48.375 mmol) and (cyanomethylene)tributylphosphorane (12.03 g, 48.375 mmol) at room temperature. The mixture was stirred at 100° C. for 12 h. After completion (monitored by TLC), the mixture was quenched with ice-cold water, extracted with DCM and concentrated under reduced pressure. The crude was purified by combi-flash chromatography using 10-15% EtOAc/Heptane to afford title compound 133 (4.50 g, 27.48%) as a pale-yellow liquid. LCMS calculated for C15H16INO2: 369.02; Found: 370 (M+1).

### Synthesis of 4-((2-(benzylthio)-4-ethylphenoxy)methyl)-2-methoxypyridine (134)

To a stirred solution of compound 133 (4.50 g, 12.18 mmol) in 1,4-Dioxane (40 mL) were added DIPEA (4.76 mL, 26.81 mmol), benzyl mercaptan (1.51 g, 12.18 mmol) and XantPhos (0.56 g, 0.97 mmol). The reaction mixture was degassed with Argon for 10 min. followed by addition of Pd₂(dba)₃ (0.57 g, 0.609 mmol). The reaction mixture was stirred at 120° C. for 6 h. After completion (monitored by TLC), the mixture was filtered through celite bed and washed with EtOAc. The filtrate was quenched with ice-cold water, extracted with EtOAc and organic layer was washed with brine, dried over anhydrous Na₂SO₄, filtered and concentrated under reduced pressure. The crude was purified by combi-flash chromatography using 10-15% of EtOAc/Heptane to afford title compound 134 (4.0 g, 80.63%) as a pale-yellow oil. LCMS calculated for C22H23NO2S: 365.14; Found: 366.20 (M+1).

### Synthesis of 5-ethyl-2-((2-methoxypyridin-4-yl)methoxy)benzenesulfonyl chloride (135)

To a stirred solution of compound 134 (1.0 g, 2.73 mmol) in ACN (10 mL), AcOH (0.50 mL) and H₂O (0.30 mL) were added 1,3-dichloro-5,5-dimethylhydantoin (1.08 g, 5.52 mmol) at 0° C. The reaction mixture was stirred at 0° C. for 2 h. After completion (monitored by TLC), the mixture was quenched with ice-cold water and extracted with EtOAc. The combined organic layer was washed with saturated NaHCO₃, dried over anhydrous Na₂SO₄, filtered and concentrated under reduced pressure to afford title compound 135 (0.60 g, 19.43%) as a brown solid which was directly used in the next step without purification. LCMS calculated for C15H16ClNO4S: 341.05; Found: 342 (M+1).

### Synthesis of tert-Butyl ((1-((3-((5-ethyl-2-((2-methoxypyridin-4-yl)methoxy)phenyl)sulfonamido)-4-methoxybenzo[d]isoxazol-6-yl)methyl)-1H-pyrazol-4-yl)methyl)carbamate (136)

To a stirred solution of compound 15 (0.06 g, 0.175 mmol) in THF (10 mL) was added 1M KOtBu in THF (0.17 mL, 1.170 mmol) at 0° C. The reaction was stirred for 10 min followed by addition of compound 135 (0.20 g, 0.585 mmol). The reaction mixture was stirred at room temperature for 3 h. After completion (monitored by TLC), the mixture was quenched with ice-cold water and extracted with EtOAc. The combined organic layer washed with brine, dried over anhydrous Na₂SO₄, filtered and concentrated under reduced pressure to afford title compound 136 (0.15 g, 20.05%) as a brown solid. LCMS calculated for C33H38N6O8S: 678.25; Found: 679.30 (M+1).

### Synthesis of N-(6-((4-(aminomethyl)-1H-pyrazol-1-yl)methyl)-4-methoxybenzo[d]isoxazol-3-yl)-5-ethyl-2-((2-methoxypyridin-4-yl)methoxy)benzenesulfonamide hydrochloride (137)

To a stirred solution of compound 136 (0.15 g, 0.220 mmol) in DCM (3 mL) was added 4M HCl in Dioxane (1 mL) at 0° C. The reaction mixture was stirred at room temperature for 4 h. After completion (monitored by TLC), the mixture was concentrated under reduced pressure. The crude was triturated with Et₂O and dried to afford title compound 137 (0.12 g, 56.52%) as a brown solid. LCMS calculated for C28H30N6O6S: 578.19; Found: 577.30 (M−1).

### Synthesis of N-((1-((3-((5-ethyl-2-((2-methoxypyridin-4-yl)methoxy)phenyl)sulfonamido)-4-methoxybenzo[d]isoxazol-6-yl)methyl)-1H-pyrazol-4-yl)methyl)-2-fluoroacrylamide (Syn. Ex. 94)

To a stirred solution of compound 137 (0.10 g, 0.162 mmol) in DCM (4 mL) were added 2-fluoroacrylic acid (0.02 g, 0.256 mmol), DIPEA (0.11 mL, 0.663 mmol) and HATU (0.12 g, 0.335 mmol) at 0° C. The reaction mixture was stirred at room temperature for 1 h. After completion (monitored by TLC), the mixture was quenched with ice-cold water and extracted with DCM. The combined organic layer was dried over anhydrous Na₂SO₄ and concentrated under reduced pressure. The crude was purified by prep. HPLC to afford N-((1-((3-((5-ethyl-2-((2-methoxypyridin-4-yl)methoxy)phenyl)sulfonamido)-4-methoxybenzo[d]isoxazol-6-yl)methyl)-1H-pyrazol-4-yl)methyl)-2-fluoroacrylamide (Syn. Ex. 94; 31.30 mg, 29.33%) as an off-white solid. (See analytical data in Table 1).

### Synthesis of methyl ((1-((3-((5-ethyl-2-((2-methoxypyridin-4-yl)methoxy)phenyl)sulfonamido)-4-methoxybenzo[d]isoxazol-6-yl)methyl)-1H-pyrazol-4-yl)methyl)carbamate (Syn. Ex. 95)

To a stirred solution of compound 137 (0.20 g, 0.325 mmol) in DCM (10 mL) were added NEt₃ (0.13 mL, 0.975 mmol) and methyl chloroformate (0.03 g, 0.390 mmol) at 0° C. The reaction mixture was stirred at 0° C. for 30 min. After completion (monitored by TLC), the mixture was diluted with water and extracted with DCM. The combined organic layer was dried over anhydrous Na₂SO₄ and concentrated under reduced pressure. The crude was purified by prep. HPLC to afford methyl((1-((3-((5-ethyl-2-((2-methoxypyridin-4-yl)methoxy)phenyl)sulfonamido)-4-methoxybenzo[d]isoxazol-6-yl)methyl)-1H-pyrazol-4-yl)methyl)carbamate (Syn. Ex. 95; 0.015 g, 7.06%) as an off-white solid. (See analytical data in Table 1).

### Examples 96-97

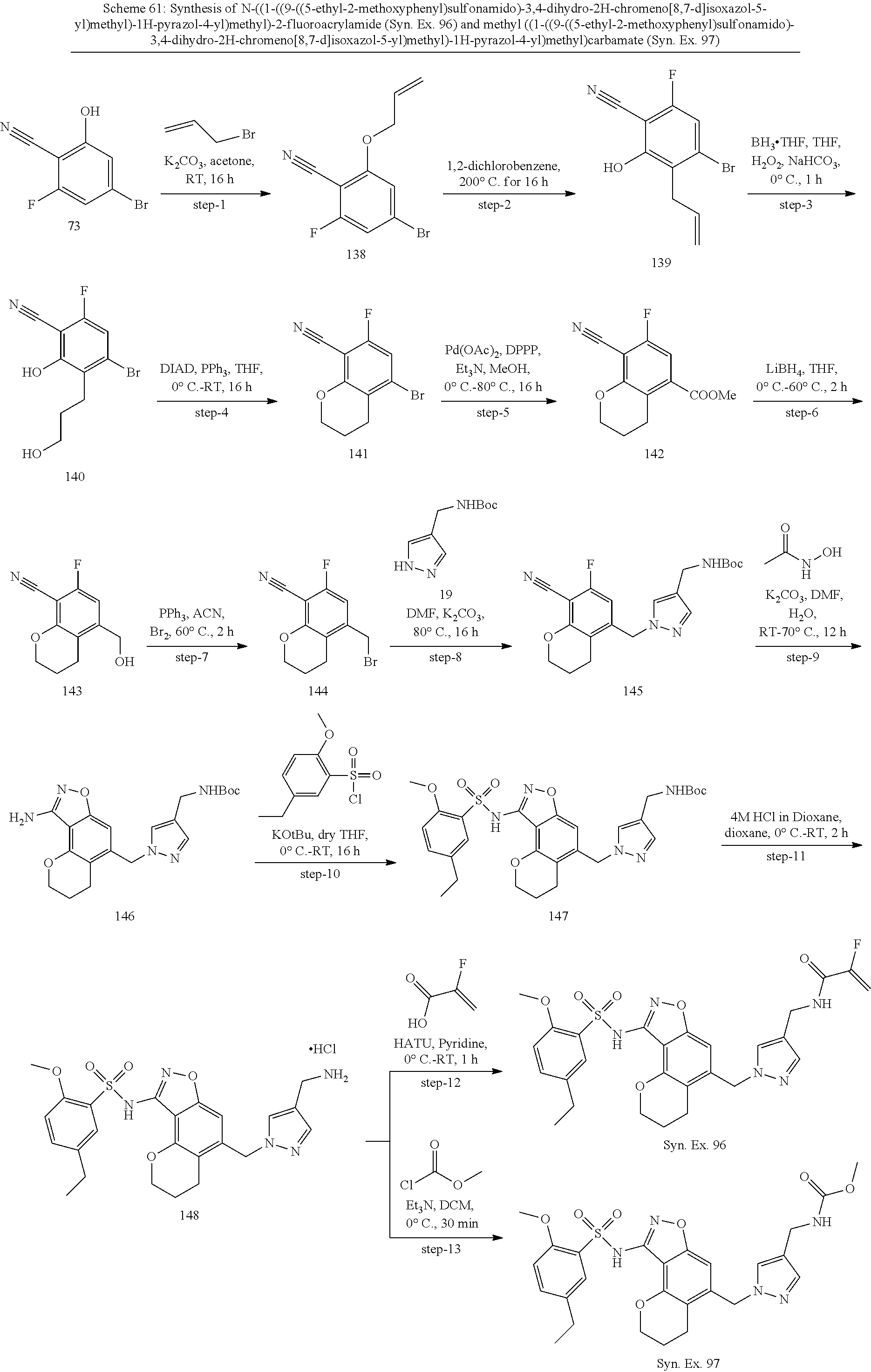

### Synthesis of 2-(allyloxy)-4-bromo-6-fluorobenzonitrile (138)

To a stirred solution of compound 73 (27 g, 124.994 mmol) in acetone (400 mL) were added K₂CO₃ (34.54 g, 249.988 mmol) and 3-bromoprop-1-ene (18 mL). The reaction mixture was stirred at room temperature for 16 h. After completion (monitored by TLC), the mixture was quenched with ice-cold water and extracted with EtOAc. The combined organic layer was dried over anhydrous Na₂SO₄, filtered and concentrated under reduced pressure to afford title compound 138 (25 g, 78.10%) as an off-white solid. ¹H NMR (400 MHz, DMSO-d₆) δ 7.48 (br d, J=8.7 Hz, 1H), 7.41 (s, 1H), 6.10-5.99 (m, 1H), 5.45 (br d, J=17.4 Hz, 1H), 5.34 (br d, J=10.4 Hz, 1H), 4.82 (br d, J=5.0 Hz, 2H).

### Synthesis of 3-allyl-4-bromo-6-fluoro-2-hydroxybenzonitrile (139)

A solution of compound 138 (25 g, 97.629 mmol) in 1,2-dichlorobenzene (200 mL) was stirred at 200° C. for 16 h. After completion (monitored by TLC), the reaction mixture was quenched with ice-cold water, extracted with EtOAc and concentrated under reduced pressure. The crude was purified by combi-flash chromatography using 25% EtOAc/Heptane to afford title compound 139 (22 g, 66%) as a white solid. ¹H NMR (400 MHz, DMSO-d₆) δ 11.44 (br s, 1H), 7.37 (d, J=9.1 Hz, 1H), 5.93-5.71 (m, 1H), 5.03 (br d, J=10.4 Hz, 1H), 4.90 (br d, J=17.0 Hz, 1H), 3.52 (br d, J=5.4 Hz, 2H).

### Synthesis of 4-bromo-6-fluoro-2-hydroxy-3-(3-hydroxypropyl)benzonitrile (140)

To a stirred solution of compound 139 (20 g, 78.103 mmol) in THF (200 mL) was added BH₃·THF complex (78 mL) at 0° C. The reaction mixture was stirred at 0° C. for 1 h followed by the addition of H₂O₂ (6.25 g, 156.207 mmol) and NaHCO₃ (14.58 g, 156.207 mmol) at 0° C. The reaction mixture was stirred at 0° C. for 1 h. After completion (monitored by TLC), the mixture was quenched with 1N HCl and extracted with EtOAc. The combined organic layer was dried over anhydrous Na₂SO₄ and concentrated under reduced pressure to afford title compound 140 (15 g, 31.53%) as a colorless liquid. LCMS calculated for C10H9BrFNO2: 272.98; Found: 274.10 (M+1).

### Synthesis of 5-bromo-7-fluorochromane-8-carbonitrile (141)

To a stirred solution of compound 140 (7.50 g, 27.363 mmol) in THF (80 mL) was added PPh₃ (10.76 g, 41.044 mmol) and DIAD (8.29 g, 41.044 mmol) at 0° C. The reaction mixture was stirred at room temperature for 16 h. After completion (monitored by TLC), the mixture was quenched with water, extracted with EtOAc and organic layer was concentrated under reduced pressure. The crude was purified by combi-flash column chromatography using 20% EtOAc/Heptane to afford title compound 141 (4.50 g, 63.58%) as an off-white solid. ¹H NMR (400 MHz, CHLOROFORM-d) δ 6.99 (d, J=7.9 Hz, 1H), 4.31-4.27 (m, 2H), 2.72 (br t, J=6.4 Hz, 2H), 2.13-2.01 (m, 2H).

### Synthesis of methyl 8-cyano-7-fluorochromane-5-carboxylate (142)

To a stirred solution of compound 141 (4.50 g, 17.573 mmol) in MeOH (100 mL) were added NEt₃ (5 mL, 35.673 mmol), DPPP (1.41 g, 3.338 mmol) followed by the addition of Pd(OAc)₂ (0.39 g, 1.664 mmol). The reaction mixture was stirred at 80° C. for 16 h under CO atmosphere (110 psi). After completion (monitored by TLC), the mixture was cooled at room temperature, filtered and concentrated under reduced pressure to afford title compound 142 (3 g, 71.85%) as a white solid. ¹H NMR (400 MHz, CHCl₃-d) δ 7.24 (s, 1H), 4.39-4.30 (m, 2H), 3.91 (s, 3H), 3.06 (t, J=6.4 Hz, 2H), 2.08-1.98 (m, 2H).

### Synthesis of 7-fluoro-5-(hydroxymethyl)chromane-8-carbonitrile (143)

To a stirred solution of compound 142 (3.30 g, 14.030 mmol) in THF (40 mL) was added LiBH₄ (0.61 g, 28.060 mmol) at 0° C. The reaction mixture was stirred at 60° C. for 2 h. After completion (monitored by TLC), the mixture was quenched with ice-cold water, extracted with EtOAc and concentrated under reduced pressure to afford title compound 143 (2.80 g, 95.35%) as an off-white solid. ¹H NMR (400 MHz, DMSO-d₆) δ 6.98 (br d, J=10.4 Hz, 1H), 5.50 (br t, J=5.2 Hz, 1H), 4.49 (br d, J=5.0 Hz, 2H), 4.28 (br t, J=5.0 Hz, 2H), 2.56 (br t, J=6.0 Hz, 2H), 2.00-1.91 (m, 2H).

### Synthesis of 5-(bromomethyl)-7-fluorochromane-8-carbonitrile (144)

To a stirred solution of compound 143 (2.80 g, 13.513 mmol) in ACN (30 mL) were added PPh₃ (5.42 g, 20.683 mmol) and Br₂ (1.04 mL, 20.372 mmol) at room temperature. The reaction mixture was stirred at 60° C. for 2 h. After completion (monitored by TLC), the mixture was quenched with sodium thiosulfate, extracted with EtOAc, dried over anhydrous Na₂SO₄ and concentrated under reduced pressure. The crude was purified by combi-flash chromatography using 15% EtOAc/Heptane to afford title compound 144 (1.30 g, 35.26%) as a white solid. ¹H NMR (400 MHz, DMSO-d₆) δ 7.15 (d, J=10.1 Hz, 1H), 4.68 (s, 2H), 4.33 (t, J=5.0 Hz, 2H), 2.77 (br t, J=6.4 Hz, 2H), 2.00 (quin, J=5.7 Hz, 2H).

### Synthesis of tert-Butyl ((1-((8-cyano-7-fluorochroman-5-yl)methyl)-1H-pyrazol-4-yl)methyl)carbamate (145)

To a stirred solution of compound 19 (0.90 g, 4.564 mmol) in DMF (30 mL) were added K₂CO₃ (1.39 g, 10.129 mmol) and compound 144 (0.90 g, 3.332 mmol). The reaction mixture was stirred at 80° C. for 16 h. After completion (monitored by TLC), the mixture was cooled at room temperature, filtered and concentrated under reduced pressure. The crude was purified by combi-flash chromatography using 20% EtOAc/Hexane to afford title compound 145 (0.70 g, 53.82%) as an off-white solid. LCMS calculated for C20H23FN4O3: 386.18; Found: 387.20 (M+1).

### Synthesis of tert-Butyl ((1-((9-amino-3,4-dihydro-2H-chromeno[8,7-d]isoxazol-5-yl)methyl)-1H-pyrazol-4-yl)methyl)carbamate (146)

To a stirred solution of compound 145 (0.60 g, 1.552 mmol) in DMF (8 mL) and water (2 mL) was added K₂CO₃ (1.28 g, 9.316 mmol) and acetohydroxamic acid (0.71 g, 9.316 mmol) at room temperature. The reaction mixture was stirred at 70° C. for 12 h. After completion (monitored by TLC), the mixture was quenched with water and extracted with EtOAc. The combined organic layer was dried over anhydrous Na₂SO₄, filtered and concentrated under reduced pressure to afford title compound 146 (0.25 g, 33.45%) as a colorless liquid. LCMS calculated for C20H25N5O4: 399.19; Found: 400.30 (M+1).

### Synthesis of tert-Butyl ((1-((9-((5-ethyl-2-methoxyphenyl)sulfonamido)-3,4-dihydro-2H-chromeno[8,7-d]isoxazol-5-yl)methyl)-1H-pyrazol-4-yl)methyl)carbamate (147)

To a stirred solution of compound 146 (0.15 g, 0.375 mmol) in dry THF (10 mL) were added 1M KOtBu in THF (1.12 mL, 1.126 mmol) and 5-ethyl-2-methoxybenzenesulfonyl chloride (0.17 g, 0.751 mmol) at 0° C. The reaction mixture was stirred at 0° C. for 16 h. After completion (monitored by TLC), the mixture was quenched with ice-cold water and extracted with EtOAc. The aqueous layer was acidified with 1N HCl to adjust the pH˜6 and extracted with EtOAc. The organic layer was washed with brine, dried over anhydrous Na₂SO₄ and concentrated under reduced pressure to afford title compound 147 (0.20 g, 80.19%) as a brown solid. LCMS calculated for C29H35N5O7S: 597.23; Found: 596.30 (M−1).

### Synthesis of N-(5-((4-(aminomethyl)-1H-pyrazol-1-yl)methyl)-3,4-dihydro-2H-chromeno[8,7-d]isoxazol-9-yl)-5-ethyl-2-methoxybenzenesulfonamide hydrochloride (148)

To a stirred solution of compound 147 (0.20 g, 0.334 mmol) in 1,4-Dioxane (5 mL) was added 4M HCl in Dioxane (0.41 mL, 1.673 mmol) at 0° C. The reaction mixture was stirred at room temperature for 2 h. After completion (monitored by TLC), the mixture was concentrated under reduced pressure. The crude was triturated with DCM, co-distilled with Et₂O and concentrated under reduced pressure to afford title compound 148 (0.12 g, 51.72%) as an off-white solid. LCMS calculated for C24H27N5O5S: 497.17; Found: 498.30 (M+1).

### Synthesis of N-((1-((9-((5-ethyl-2-methoxyphenyl)sulfonamido)-3,4-dihydro-2H-chromeno[8,7-d]isoxazol-5-yl)methyl)-1H-pyrazol-4-yl)methyl)-2-fluoroacrylamide (Syn. Ex. 96)

To a stirred solution of 2-fluoroacrylic acid (0.02 g, 0.280 mmol) in pyridine (8 mL) were added HATU (0.14 g, 0.394 mmol) and compound 148 (0.10 g, 0.187 mmol) at 0° C. The reaction mixture was stirred at room temperature for 1 h. After completion (monitored by TLC), the mixture was quenched with water and extracted with EtOAc. The combined organic layer was dried over anhydrous Na₂SO₄, filtered and concentrated under reduced pressure. The crude was purified by prep. HPLC to afford N-((1-((9-((5-ethyl-2-methoxyphenyl)sulfonamido)-3,4-dihydro-2H-chromeno[8,7-d]isoxazol-5-yl)methyl)-1H-pyrazol-4-yl)methyl)-2-fluoroacrylamide (Syn. Ex. 96; 47 mg, 44.09%) as an off-white solid. (See analytical data in Table 1).

### Synthesis of methyl ((1-((9-((5-ethyl-2-methoxyphenyl)sulfonamido)-3,4-dihydro-2H-chromeno[8,7-d]isoxazol-5-yl)methyl)-1H-pyrazol-4-yl)methyl)carbamate (Syn. Ex. 97)

To a stirred solution of compound 148 (0.08 g, 0.149 mmol) in DCM (5 mL) were added Et₃N (0.04 mL, 0.299 mmol) and methyl chloroformate (0.01 g, 0.149 mmol) in DCM at 0° C. The reaction mixture was stirred at 0° C. for 30 min. After completion (monitored by TLC), the mixture was quenched with water and extracted with DCM. The combined organic layer was dried over anhydrous Na₂SO₄, filtered and concentrated under reduced pressure. The crude was purified by prep. HPLC to afford methyl ((1-((9-((5-ethyl-2-methoxyphenyl)sulfonamido)-3,4-dihydro-2H-chromeno[8,7-d]isoxazol-5-yl)methyl)-1H-pyrazol-4-yl)methyl)carbamate (Syn. Ex. 97; 12 mg, 13.84%) as an off-white solid. (See analytical data in Table 1).

### Example 98

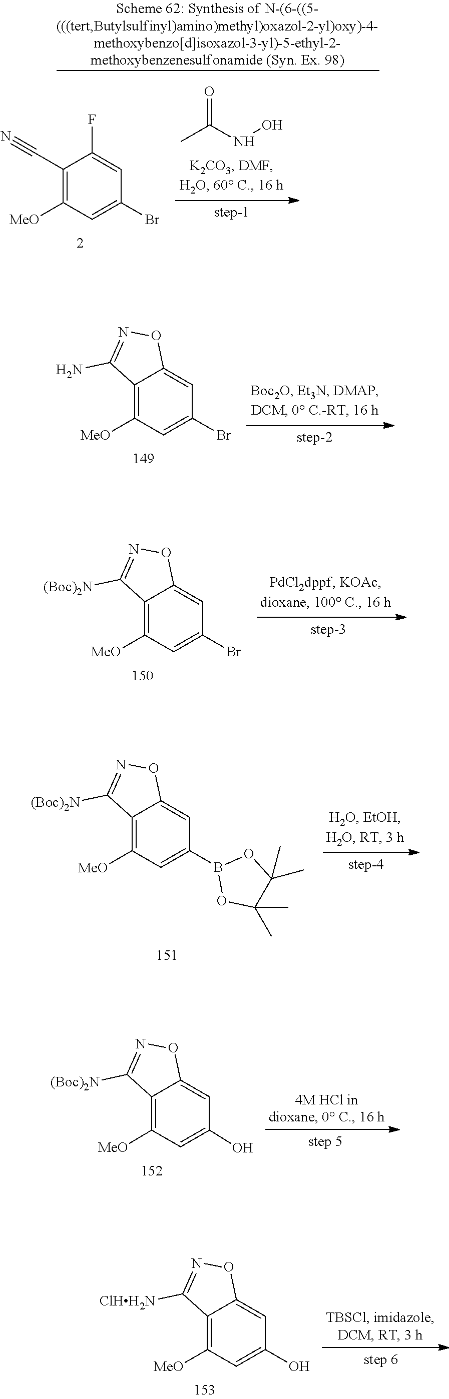

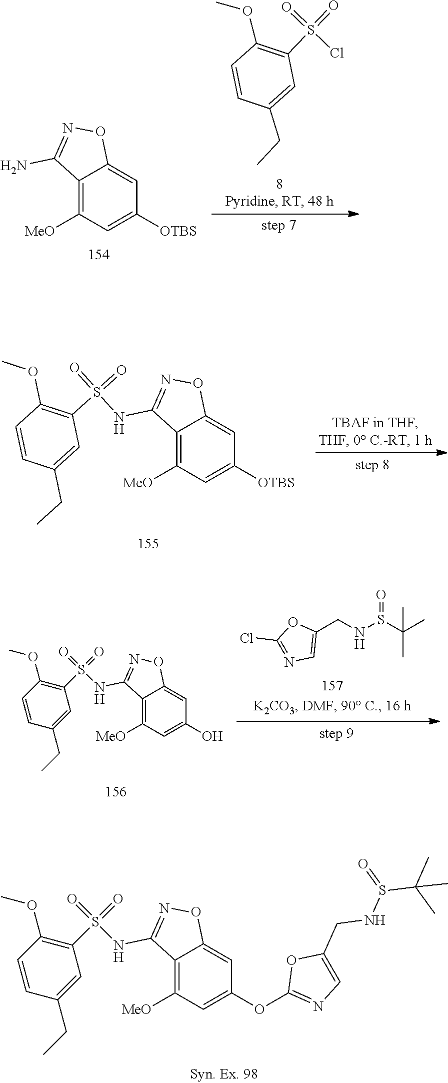 [SMILES: COc1cc(O[Si](C)(C)C(C)(C)C)cc2onc(N)c12 ; CCc1ccc(OC)c(S(=O)(=O)Cl)c1 ; CCc1ccc(OC)c(S(=O)(=O)Nc2noc3cc(O[Si](C)(C)C(C)(C)C)cc(OC)c23)c1 ; CCc1ccc(OC)c(S(=O)(=O)Nc2noc3cc(O)cc(OC)c23)c1 ; CC(C)(C)S(=O)NCc1cnc(Cl)o1 ; CCc1ccc(OC)c(S(=O)(=O)Nc2noc3cc(Oc4ncc(CNS(=O)C(C)(C)C)o4)cc(OC)c23)c1]

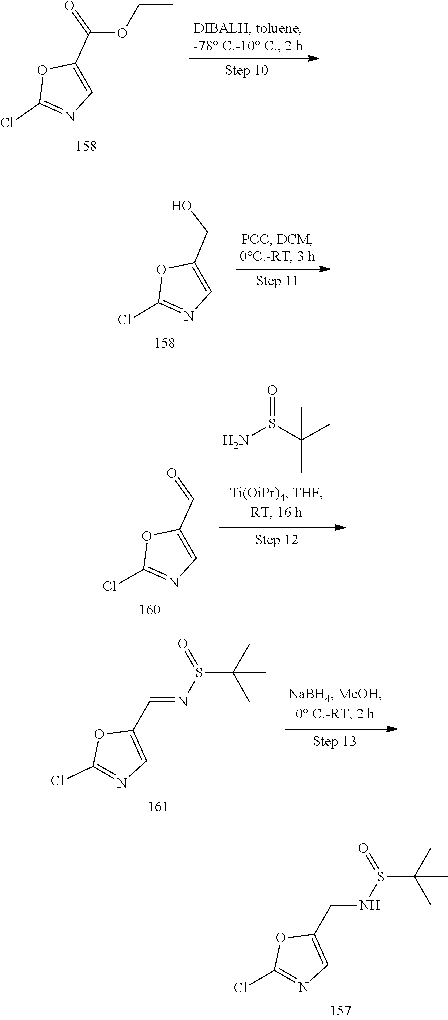

### Synthesis of 6-bromo-4-methoxybenzo[d]isoxazol-3-amine (149)

To a stirred solution of compound 2 (25 g, 108.676 mmol) in DMF (550 mL) was added acetohydroxamic acid (24.97 g, 326.030 mmol) and K₂CO₃ (45.01 g, 326.030 mmol) in water (80 mL) at room temperature. The reaction mixture was stirred at 60° C. for 16 h. After completion (monitored by TLC), the mixture was quenched with ice-cold water and extracted with EtOAc. The combined organic layer was dried over anhydrous Na₂SO₄, filtered and concentrated under reduced pressure to afford title compound 149 (16 g, 38.22%) as an off-white solid. ¹H NMR (400 MHz, DMSO-d₆) δ ppm 7.35 (s, 1H), 7.13 (br s, 1H), 5.92 (s, 2H), 3.86 (s, 3H).

### Synthesis of tert-Butyl (6-bromo-4-methoxybenzo[d]isoxazol-3-yl)(tert-butoxycarbonyl)carbamate (150)

To a stirred solution of compound 149 (8 g, 32.913 mmol) in DCM (120 mL) were added Et₃N (13.77 mL, 98.741 mmol), 4-dimethylaminopyridine (0.80 g, 6.582 mmol) and di-tert-butyl dicarbonate (21.76 g, 98.741 mmol) at 0° C. The mixture was stirred at room temperature for 16 h. After completion (monitored by TLC), the mixture was quenched with ice-cold water and extracted with DCM. The combined organic layer was dried over anhydrous Na₂SO₄, filtered and concentrated under reduced pressure to afford title compound 150 (5 g, 33.58%) as an off-white solid. LCMS calculated for C18H₂₃BrN₂O₆: 442.07; Found: 286.90 [M-(Boc+tBu)+1].

### Synthesis of tert-Butyl (tert-butoxycarbonyl)(4-methoxy-6-(4,4,5,5-tetramethyl-1,3,2-dioxaborolan-2-yl)benzo[d]isoxazol-3-yl)carbamate (151)

To a stirred solution of compound 150 (10 g, 22.55 mmol) in 1,4-Dioxane (100 mL) was added bis(pinacolato)diboron (11.81 g, 45.11 mmol) and KOAc (4.47 g, 45.11 mmol). The reaction was degassed with N₂ purging for 10 min followed by addition of PdCl₂(dppf) (0.86 g, 1.187 mmol). The mixture was stirred at 100° C. for 16 h. After completion (monitored by TLC), the mixture was filtered through a celite bed and washed with EtOAc. The filtrate was quenched with water and extracted with EtOAc. The combined organic layer was dried over anhydrous Na₂SO₄, filtered and concentrated under reduced pressure. The crude was purified by combi-flash chromatography using 15-18% EtOAc/Heptane to afford title compound 151 (6.0 g, 33.52%) as an off-white solid. LCMS calculated for C₂₄H35BN2O8: 490.25; Found: 335.20 [M-(Boc+tBu+1)].

### Synthesis of tert-Butyl (tert-butoxycarbonyl)(6-hydroxy-4-methoxybenzo[d]isoxazol-3-yl)carbamate (152)

To a stirred solution of compound 151 (6.0 g, 14.698 mmol) in EtOH (66 mL) and H₂O (30 mL) was added 30% H₂O₂ (40.53 mL, 396.85 mmol) at 0° C. The mixture was stirred at room temperature for 3 h. After completion (monitored by TLC), the mixture was quenched with Na₂CO₃ and extracted with EtOAc. The combined organic layer was washed with brine, dried over anhydrous Na₂SO₄, filtered and concentrated under reduced pressure. The crude was purified by combi-flash chromatography using 40% EtOAc/Heptane to afford title compound 152 (4.0 g, 70.82%) as a brown solid. LCMS calculated for C18H₂₄N₂O₇: 380.16; Found: 379.0 (M−1).

### Synthesis of 3-amino-4-methoxybenzo[d]isoxazol-6-ol hydrochloride (153)

To a stirred solution of compound 152 (0.50 g, 1.31 mmol) in DCM (5 mL) was added 4M HCl in dioxane (1.64 mL, 6.57 mmol) at 0° C. The mixture was stirred at room temperature for 16 h. After completion (monitored by TLC), the mixture was concentrated under reduced pressure. The crude was triturated with Et₂O to afford title compound 153 (0.22 g, 76.42%) as a brown solid. LCMS calculated for C8H8N2O3: 180.05; Found: 181.0 (M+1).

### Synthesis of 6-((tert-Butyldimethylsilyl)oxy)-4-methoxybenzo[d]isoxazol-3-amine (154)

To a stirred solution of compound 153 (0.22 g, 1.01 mmol) in THF (5 mL) were added imidazole (0.20 g, 3.04 mmol) and tert-butyldimethylchlorosilane (0.18 g, 1.21 mmol) at 0° C. The mixture was stirred at room temperature for 3 h. After completion (monitored by TLC), the mixture was quenched with ice-cold water and extracted with EtOAc. The combined organic layer was washed with brine, dried over anhydrous Na₂SO₄ and concentrated under reduced pressure. The crude was purified by combi-flash chromatography using 22-27% EtOAc/Heptane to afford title compound 154 (0.32 g) as a white solid. LCMS calculated for C14H22N2O3Si: 294.14; Found: 295.20 (M+1).

### Synthesis of N-(6-((tert-Butyldimethylsilyl)oxy)-4-methoxybenzo[d]isoxazol-3-yl)-5-ethyl-2-methoxybenzenesulfonamide (155)

To a stirred solution of compound 154 (0.15 g, 0.509 mmol) in pyridine (3 mL) was added 5-ethyl-2-methoxybenzenesulfonyl chloride (0.17 g, 0.764 mmol). The mixture was stirred at room temperature for 48 h. After completion (monitored by TLC), the mixture was quenched with ice-cold water, acidified with 1N HCl and extracted with EtOAc. The combined organic layer was washed with brine, dried over anhydrous Na₂SO₄, filtered and concentrated under reduced pressure. The crude was purified by combi-flash chromatography using 28-32% EtOAc/Heptane to afford title compound 155 (0.14 g, 55.77%) as a brown solid. LCMS calculated for C23H32N2O6SSi: 492.18; Found: 493.20 (M+1).

### Synthesis of 5-ethyl-N-(6-hydroxy-4-methoxybenzo[d]isoxazol-3-yl)-2-methoxy benzenesulfonamide (156)

To a stirred solution of compound 155 (0.50 g, 1.014 mmol) in THF (6 mL) was added 1M TBAF in THF (1.11 mL, 1.116 mmol) at 0° C. The mixture was stirred at room temperature for 1 h. After completion (monitored by TLC), the mixture was quenched with ice-cold water and extracted with EtOAc. The combined organic layer was washed with brine, dried over anhydrous Na₂SO₄, filtered and concentrated under reduced pressure to afford title compound 156 (0.38 g, 91.14%) as a brown solid. LCMS calculated for C₁₇H18N2O6S: 378.09; Found: 379.10 (M+1).

### Synthesis of (2-chlorooxazol-5-yl)methanol (158)

To a stirred solution of compound 158 (5 g, 28.47 mmol) in THF (60 mL) was added 1M DIBAL-H in Toluene (56.95 mL, 56.95 mmol) at −78° C. The mixture was cooled to −10° C. and stirred for 2 h. After completion (monitored by TLC), the mixture was quenched with saturated aqueous NH₄Cl, diluted with EtOAc, filtered through a celite bed and extracted with EtOAc. The combined organic layer was washed with brine, dried over anhydrous Na₂SO₄, filtered and concentrated under reduced pressure to afford title compound 159 (2.45 g, 60.31%) as a brown oil. LCMS calculated for C4H4ClNO2: 132.99; Found: 134.0 (M+1).

### Synthesis of 2-chlorooxazole-5-carbaldehyde (160)

To a stirred solution of compound 159 (2.50 g, 18.72 mmol) in DCM (30 mL) was added PCC (6.17 g, 28.083 mmol) at 0° C. The mixture was stirred at room temperature for 3 h. After completion (monitored by TLC), the mixture was filtered through a celite pad and washed with DCM. The filtrate was washed with water, organic layer was dried over anhydrous Na₂SO₄, filtered and concentrated under reduced pressure to afford title compound 160 (1.20 g, 40.93%) as a brown solid. LCMS calculated for C4H2ClNO2: 130.98; Found: 129.20 (M−2).

### Synthesis of (E)-N-((2-chlorooxazol-5-yl)methylene)-2-methylpropane-2-sulfinamide (161)

To a stirred solution of compound 160 (1.20 g, 9.12 mmol) in THF (15 mL) were added 2-methylpropane-2-sulfinamide (2.21 g, 18.24 mmol) and Ti(OiPr)₄ (5.34 g, 18.24 mmol) at 0° C. The mixture was stirred at room temperature for 16 h. After completion (monitored by TLC), the mixture was quenched with saturated NH₄Cl, diluted with EtOAc, filtered through a celite bed and extracted with EtOAc. The combined organic layer was washed with brine, dried over anhydrous Na₂SO₄, filtered and concentrated under reduced pressure. The crude was purified by combi-flash chromatography using 13-16% EtOAc/Heptane to afford title compound 161 (1.20 g, 55.28%) as a white solid. LCMS calculated for C8H11ClN202S: 234.02; Found: 235.0 (M+1).

### Synthesis of N-((2-chlorooxazol-5-yl)methyl)-2-methylpropane-2-sulfinamide (157)

To a stirred solution of compound 161 (1.20 g, 5.112 mmol) in MeOH (15 mL) was added NaBH₄ (0.38 g, 10.225 mmol) at 0° C. The mixture was stirred at room temperature for 2 h. After completion (monitored by TLC), the mixture was quenched with saturated NH₄Cl, concentrated under reduced pressure and extracted with EtOAc. The combined organic layer was washed with brine, dried over anhydrous Na₂SO₄, filtered and concentrated under reduced pressure to afford title compound 157 (1.10 g, 90.16%) as a brown oil. LCMS calculated for C8H13ClN202S: 236.04; Found: 237.10 (M+1).

### Synthesis of N-(6-((5-(((tert-Butylsulfinyl)amino)methyl)oxazol-2-yl)oxy)-4-methoxybenzo[d]isoxazol-3-yl)-5-ethyl-2-methoxybenzenesulfonamide (Syn. Ex. 98)

To a stirred solution of compound 156 (0.20 g, 0.528 mmol) in DMF (2 mL) were added K₂CO₃ (0.21 g, 1.585 mmol) and compound 157 (0.18 g, 0.792 mmol) at room temperature. The mixture was stirred at 90° C. for 16 h. After completion (monitored by TLC), the mixture was quenched with ice-cold water, acidified with 1N HCl to pH ˜5 and extracted with EtOAc. The combined organic layer was washed with brine, dried over anhydrous Na₂SO₄, filtered and concentrated under reduced pressure. The crude was purified by combi-flash chromatography using 26-30% EtOAc/Heptane to afford 0.17 g of desired product as a brown solid which was further purified by prep. HPLC to afford N-(6-((5-(((tert-Butylsulfinyl)amino)methyl)oxazol-2-yl)oxy)-4-methoxy benzo[d]isoxazol-3-yl)-5-ethyl-2-methoxybenzenesulfonamide (Syn. Ex. 98; 5.50 mg, 18.15%) as a brown solid. (See analytical data in Table 1).

### Examples 99-100

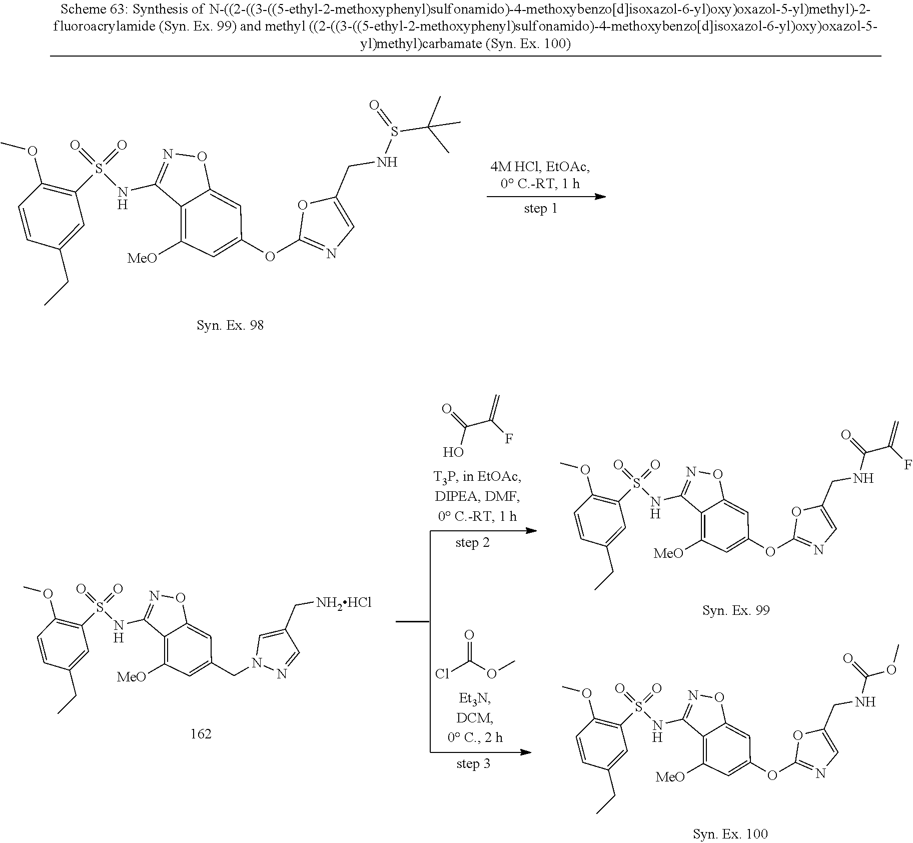

### Synthesis of N-(6-((5-(aminomethyl)oxazol-2-yl)oxy)-4-methoxybenzo[d]isoxazol-3-yl)-5-ethyl-2-methoxybenzenesulfonamide hydrochloride (162)

To a stirred solution of Syn. Ex. 98 (0.10 g, 0.172 mmol) in EtOAc (3 mL) was added 4M HCl in 1,4-Dioxane (0.043 mL, 0.172 mmol) at 0° C. The reaction mixture was stirred at room temperature for 1 h. After completion (monitored by TLC), the mixture was concentrated under reduced pressure, triturated with EtOAc, filtered and dried to afford title compound 162 (50 mg, 49.30%) as a brown solid. LCMS calculated for C21H22N4O7S: 474.12; Found: 473.20 (M−1).

### Synthesis of N-((2-((3-((5-ethyl-2-methoxyphenyl)sulfonamido)-4-methoxybenzo[d]isoxazol-6-yl)oxy)oxazol-5-yl)methyl)-2-fluoroacrylamide (Syn. Ex. 99)

To a stirred solution of compound 162 (0.025 g, 0.052 mmol) in DMF (1 mL) were added DIPEA (0.027 mL, 0.158 mmol), 2-fluoroacrylic acid 2 (0.0056 g, 0.063 mmol) and 50% T₃P in EtOAc (0.10 g, 0.158 mmol) at 0° C. The mixture was stirred at room temperature for 1 h. After completion (monitored by TLC), the mixture was quenched with ice-cold water and extracted with EtOAc. The combined organic layer was dried over anhydrous Na₂SO₄ and concentrated under reduced pressure. The crude was submitted for prep. HPLC to afford N-((2-((3-((5-ethyl-2-methoxyphenyl)sulfonamido)-4-methoxybenzo[d]isoxazol-6-yl)oxy)oxazol-5-yl)methyl)-2-fluoroacrylamide (Syn. Ex. 99; 3 mg, 10.12%) as an off-white solid. (See analytical data in Table 1).

### Synthesis of methyl ((2-((3-((5-ethyl-2-methoxyphenyl)sulfonamido)-4-methoxybenzo[d]isoxazol-6-yl)oxy)oxazol-5-yl)methyl)carbamate (Syn. Ex. 100)

To a stirred solution of compound 162 (0.05 g, 0.097 mmol) in DCM (3 mL) was added NEt₃ (0.04 mL, 0.293 mmol) and methyl chloroformate (0.01 g, 0.107 mmol) at 0° C. The reaction mixture was stirred at room temperature for 2 h. After completion (monitored by TLC), the mixture was quenched with ice-cold water, acidified with 1N HCl and extracted with EtOAc. The combined organic layer was washed with brine, dried over anhydrous Na₂SO₄ and concentrated under reduced pressure. The crude was purified by prep. HPLC to afford methyl ((2-((3-((5-ethyl-2-methoxyphenyl)sulfonamido)-4-methoxybenzo[d]isoxazol-6-yl)oxy)oxazol-5-yl)methyl)carbamate (Syn. Ex. 100; 6 mg, 11.45%) as a light-brown solid. (See analytical data in Table 1).

### Examples 101-103

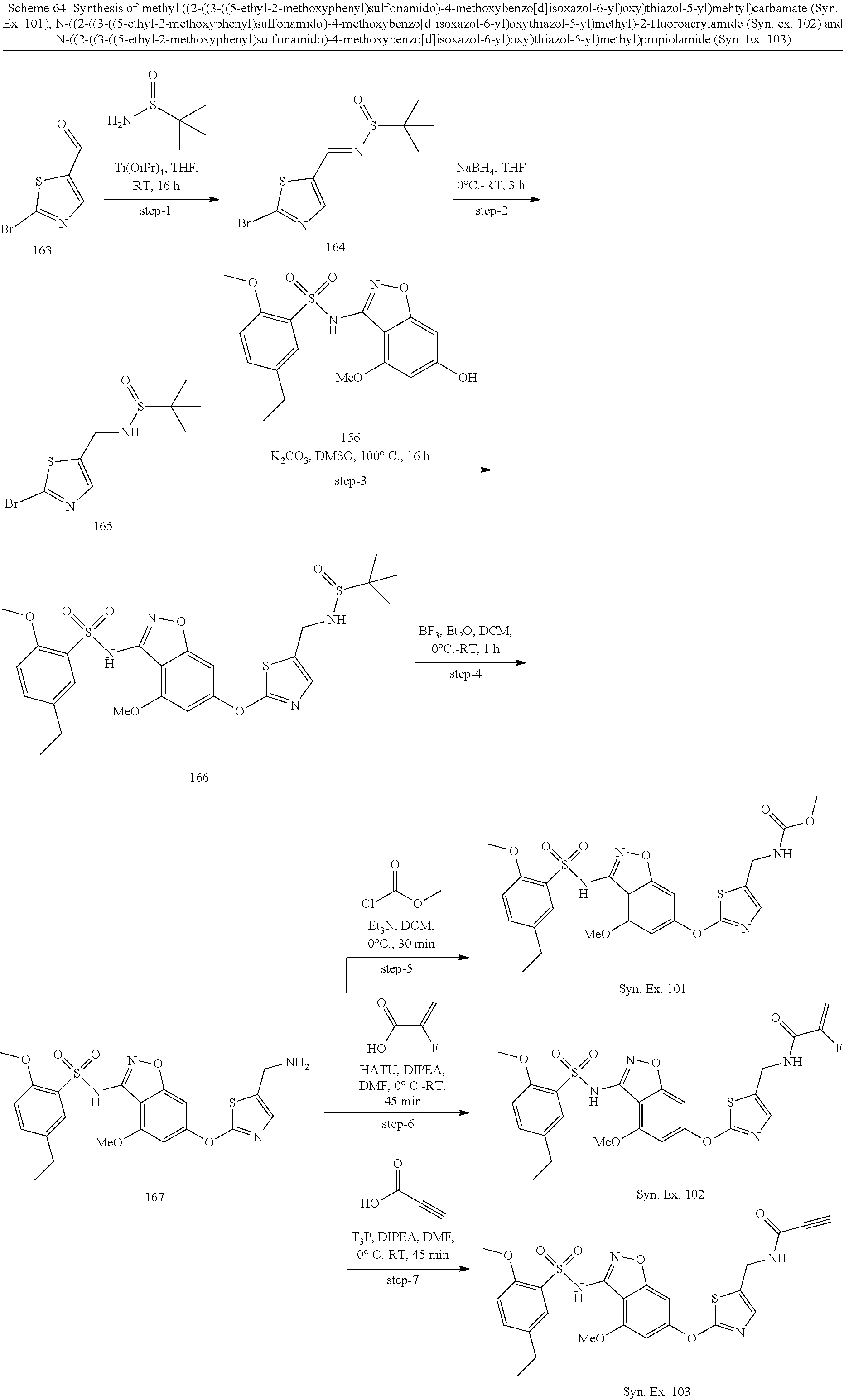

### Synthesis of (E)-N-((2-bromothiazol-5-yl)methylene)-2-methylpropane-2-sulfinamide (164)

To a stirred solution of compound 163 (5 g, 26.037 mmol) in THF (50 mL) was added 2-methylpropane-2-sulfinamide (6.31 g, 52.075 mmol) and Ti(OiPr)₄ (15.25 g, 52.075 mmol) at 0° C. The mixture was stirred at room temperature for 16 h. After completion (monitored by TLC), the mixture was quenched with ice-cold water, filtered and extracted with EtOAc. The combined organic layer was dried over anhydrous Na₂SO₄, filtered and concentrated under reduced pressure. The crude was purified by combi-flash chromatography using 35-45% EtOAc/Heptane to afford title compound 164 (6.50 g, 83.71%) as a white solid. LCMS calculated for C8H11BrN2OS2: 293.95; Found: 294.90 (M+1).

Synthesis of N-((2-bromothiazol-5-yl)methyl)-2-methylpropane-2-sulfinamide (165) To a stirred solution of compound 164 (6 g, 20.324 mmol) in THF (60 mL) was added NaBH₄ (1.60 g, 40.649 mmol) at 0° C. The mixture was stirred at room temperature for 3 h. After completion (monitored by TLC), the mixture was quenched with ice-cold water and extracted with EtOAc. The combined organic layer was dried over anhydrous Na₂SO₄, filtered and concentrated under reduced pressure to afford title compound 165 (4 g, 60.25%) as a white solid. LCMS calculated for C8H13BrN2OS2: 295.97; Found: 298.90 (M+3).

### Synthesis of N-(6-((5-(((tert-Butylsulfinyl)amino)methyl)thiazol-2-yl)oxy)-4-methoxybenzo[d]isoxazol-3-yl)-5-ethyl-2-methoxybenzenesulfonamide (166)

To a stirred solution of compound 156 (0.15 g, 0.396 mmol) in DMSO (2 mL) was added compound 165 (0.16 g, 0.554 mmol) and K₂CO₃ (0.16 g, 1.189 mmol). The reaction mixture was stirred at 100° C. for 16 h. After completion (monitored by TLC), the mixture was quenched with ice-cold water and extracted with EtOAc. The organic layer was dried over anhydrous Na₂SO₄, filtered and concentrated under reduced pressure. The crude was purified by combi-flash chromatography using 10-15% MeOH/DCM to afford title compound 166 (0.17 g, 34.61%) as a brown semi-solid. LCMS calculated for C25H30N4O7S3: 594.13; Found: 595.10 (M+1).

### Synthesis of N-(6-((5-(aminomethyl)thiazol-2-yl)oxy)-4-methoxybenzo[d]isoxazol-3-yl)-5-ethyl-2-methoxybenzenesulfonamide (167)

To a stirred solution of compound 166 (0.02 g, 0.033 mmol) in DCM (1 mL) was added BF₃·Et₂O (0.014 g, 0.050 mmol) at 0° C. The reaction mixture was stirred at room temperature for 1 h. After completion (monitored by TLC), the mixture was concentrated under reduced pressure. The crude was triturated with Et₂O to afford title compound 167 (0.01 g, 43.04%) as a brown semi-solid. LCMS calculated for C21H22N4O6S2: 490.10; Found: 489.10 (M−1).

### Synthesis of methyl ((2-((3-((5-ethyl-2-methoxyphenyl)sulfonamido)-4-methoxybenzo[d]isoxazol-6-yl)oxy)thiazol-5-yl)methyl)carbamate (Syn. Ex. 101)

To a stirred solution of compound 167 (0.13 g, 0.265 mmol) in DCM (3 mL) were added Et₃N (1.09 mL, 0.795 mmol) and methyl chloroformate (0.03 g, 0.318 mmol) at 0° C. The reaction mixture was stirred at 0° C. for 30 min. After completion (monitored by TLC), the mixture was quenched with ice-cold water and extracted with DCM. The combined organic layer was dried over anhydrous Na₂SO₄ and concentrated under reduced pressure. The crude was purified by prep. HPLC to afford methyl((2-((3-((5-ethyl-2-methoxyphenyl) sulfonamido)-4-methoxybenzo[d]isoxazol-6-yl)oxy)thiazol-5-yl)methyl)carbamate (Syn. Ex. 101; 5 mg, 3.33%) as an off-white solid.

### Synthesis of N-((2-((3-((5-ethyl-2-methoxyphenyl)sulfonamido)-4-methoxybenzo[d]isoxazol-6-yl)oxy)thiazol-5-yl)methyl)-2-fluoroacrylamide (Syn. Ex. 102)

To a stirred solution of N-(6-((5-(aminomethyl)thiazol-2-yl)oxy)-4-methoxybenzo[d]isoxazol-3-yl)-5-ethyl-2-methoxybenzenesulfonamide 167 (0.13 g, 0.265 mmol) in DMF (3 mL) were added DIPEA (0.13 mL, 0.795 mmol), 2-fluoroacrylic acid (0.047 g, 0.530 mmol) and HATU (0.15 g, 0.397 mmol) at 0° C. The reaction mixture was stirred at room temperature for 45 min. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-((2-((3-((5-ethyl-2-methoxyphenyl) sulfonamido)-4-methoxybenzo[d]isoxazol-6-yl)oxy)thiazol-5-yl)methyl)-2-fluoroacrylamide (Syn. Ex. 102; 7 mg, 4.60%) as an off-white solid.

### Synthesis of N-((2-((3-((5-ethyl-2-methoxyphenyl)sulfonamido)-4-methoxybenzo[d]isoxazol-6-yl)oxy)thiazol-5-yl)methyl)propiolamide (Syn. Ex. 103)

To a stirred solution of compound 167 (0.13 g, 0.265 mmol) in DMF (1 mL) were added DIPEA (0.13 mL, 0.795 mmol), propiolic acid (0.037 g, 0.530 mmol) followed by the addition of and 50% T₃P in EtOAc (0.84 mL, 1.325 mmol) at 0° C. The reaction mixture was stirred at room temperature for 45 min. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-((2-((3-((5-ethyl-2-methoxyphenyl)sulfonamido)-4-methoxybenzo[d]isoxazol-6-yl)oxy)thiazol-5-yl)methyl)propiolamide (Syn. Ex. 103; 6 mg, 4.17%) as an off-white solid. (See analytical data in Table 1).

### Examples 104-105

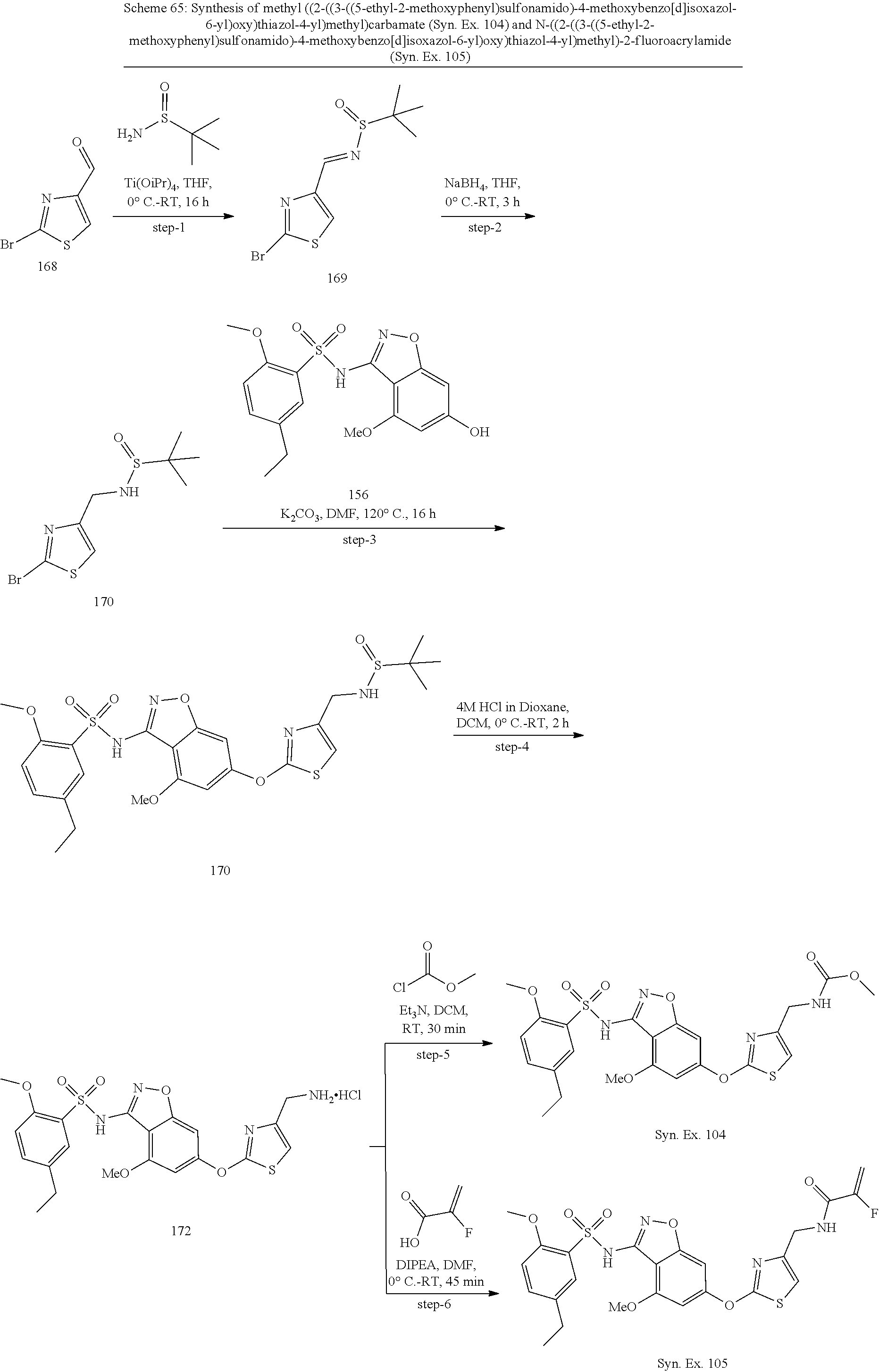

### Synthesis of (Z)—N-((2-bromothiazol-4-yl)methylene)-2-methylpropane-2-sulfinamide (169)

To a stirred solution of compound 168 (5 g, 26.037 mmol) in THF (50 mL) were added 2-methylpropane-2-sulfinamide (6.31 g, 52.075 mmol) and Ti(OiPr)₄ (15.25 g, 52.075 mmol) at 0° C. The reaction mixture was stirred at room temperature for 16 h. After completion (monitored by TLC), the mixture was quenched with water and extracted with EtOAc. The combined organic layer was dried over anhydrous Na₂SO₄, filtered and concentrated under reduced pressure. The crude was purified by combi-flash chromatography using 35-45% EtOAc/Heptane to afford title compound 169 (6.50 g, 83.71%) as a white solid. LCMS calculated for C8H11BrN23S2: 293.95; Found: 294.90 (M+1).

### Synthesis of N-((2-bromothiazol-4-yl)methyl)-2-methylpropane-2-sulfinamide (170)

To a stirred solution of compound 169 (6 g, 20.324 mmol) in THF (60 mL) was added NaBH₄ (1.60 g, 40.649 mmol) at 0° C. The reaction mixture was stirred at room temperature for 3 h. After completion (monitored by TLC), the mixture was quenched with water and extracted with EtOAc. The organic layer was dried over anhydrous Na₂SO₄, filtered and concentrated under reduced pressure to afford title compound 170 (4 g, 60.26%) as a white solid. LCMS calculated for C8H13BrN2OS2: 295.97; Found: 298.90 (M+3).

### Synthesis of N-(6-((4-(((tert-Butylsulfinyl)amino)methyl)thiazol-2-yl)oxy)-4-methoxybenzo[d]isoxazol-3-yl)-5-ethyl-2-methoxybenzenesulfonamide (171)

To a stirred solution of compound 156 (0.15 g, 0.396 mmol) in DMF (2 mL) was added compound 170 (0.21 g, 0.713 mmol) followed by the addition of K₂CO₃ (0.16 g, 1.189 mmol). The mixture was stirred at 120° C. for 16 h. After completion (monitored by TLC), the mixture was purified by combi-flash chromatography using 90% EtOAc/Heptane to afford title compound 171 (0.10 g, 27.57%) as a yellow solid. LCMS calculated for C25H30N4O7S3: 594.13; Found: 595.20 (M+1).

### Synthesis of N-(6-((4-(aminomethyl)thiazol-2-yl)oxy)-4-methoxybenzo[d]isoxazol-3-yl)-5-ethyl-2-methoxybenzenesulfonamide hydrochloride (172)

To a stirred solution of compound 171 (0.10 g, 0.168 mmol) in DCM (1 mL) was added 4M HCl in Dioxane (1 mL, 4 mmol) at 0° C. The mixture was stirred at room temperature for 2 h. After completion (monitored by TLC), the mixture was concentrated under reduced pressure. The crude was washed with DCM, co-distilled with Et₂O and concentrated under reduced pressure to afford title compound 172 (0.08 g, 63.64%) as an off-white solid. LCMS calculated for C21H22N4O6S2: 490.10; Found: 491.10 (M+1).

### Synthesis of methyl ((2-((3-((5-ethyl-2-methoxyphenyl)sulfonamido)-4-methoxybenzo[d]isoxazol-6-yl)oxy)thiazol-4-yl)methyl)carbamate (Syn. Ex. 104)

To a stirred solution of compound 172 (0.17 g, 0.322 mmol) in DCM (1.80 mL) was added NEt₃ (0.0089 mL, 0.064 mmol) followed by the addition of methyl chloroformate (0.016 g, 0.161 mmol) in DCM at 0° C. The mixture was stirred at room temperature for 30 min. After completion (monitored by TLC), the mixture was concentrated under reduced pressure. The crude was purified by prep. HPLC to afford methyl ((2-((3-((5-ethyl-2-methoxyphenyl)sulfonamido)-4-methoxybenzo[d]isoxazol-6-yl)oxy)thiazol-4-yl)methyl)carbamate (Syn. Ex. 104; 9 mg, 5.07%) as a white solid.

### Synthesis of N-((2-((3-((5-ethyl-2-methoxyphenyl)sulfonamido)-4-methoxybenzo[d]isoxazol-6-yl)oxy)thiazol-4-yl)methyl)-2-fluoroacrylamide (Syn. Ex. 105)

To a stirred solution of compound 172 (0.15 g, 0.284 mmol) in DMF (5 mL) were added 2-fluoroacrylic acid (0.03 g, 0.341 mmol), DIPEA (0.10 mL, 0.569 mmol) and HATU (0.22 g, 0.569 mmol) at 0° C. The reaction mixture was stirred at room temperature for 45 min. After completion (monitored by TLC), the mixture was quenched with ice-cold water (3 mL) and extracted with DCM. The combined organic layer was dried over anhydrous Na₂SO₄, filtered and concentrated under reduced pressure. The crude was purified by prep. HPLC to afford N-((2-((3-((5-ethyl-2-methoxyphenyl)sulfonamido)-4-methoxybenzo[d]isoxazol-6-yl)oxy)thiazol-4-yl)methyl)-2-fluoroacrylamide (Syn. Ex. 105; 40 mg, 24.98%) as an off-white solid. (See analytical data in Table 1).

### Example 106

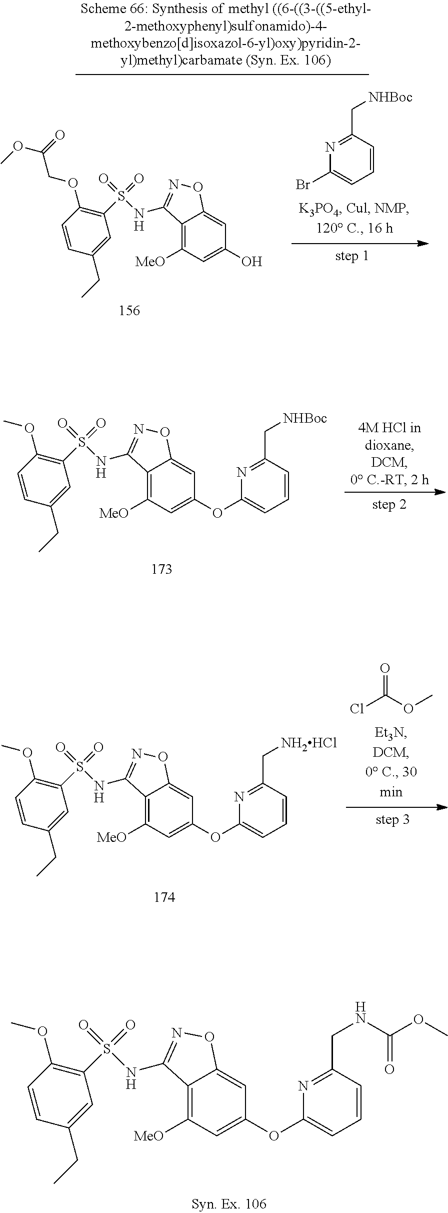

### Synthesis of tert-Butyl ((6-((3-((5-ethyl-2-methoxyphenyl)sulfonamido)-4-methoxybenzo[d]isoxazol-6-yl)oxy)pyridin-2-yl)methyl)carbamate (173)

To a stirred solution of compound 156 (0.20 g, 0.528 mmol) in NMP (4 mL) were added tert-Butyl ((6-bromopyridin-2-yl)methyl)carbamate (0.22 g, 0.792 mmol) and potassium K₃PO₄ (0.35 g, 1.585 mmol). The mixture was degassed for 10 min with Argon followed by addition of picolinic acid (0.013 g, 0.105 mmol) and CuI (0.003 g, 0.015 mmol). The reaction mixture was stirred at 120° C. for 16 h. After completion (monitored by TLC), the mixture was quenched with saturated ice-cold water and extracted with EtOAc. The combined organic layer was washed with brine, dried over anhydrous Na₂SO₄, filtered and concentrated under reduced pressure. The crude was purified by combi-flash chromatography using 54-65% EtOAc/Heptane to afford title compound 173 (0.11 g, 17.94%) as a brown solid. LCMS calculated for C28H32N4O8S: 584.19; Found: 583.20 (M−1).

### Synthesis of N-(6-((6-(aminomethyl)pyridin-2-yl)oxy)-4-methoxybenzo[d]isoxazol-3-yl)-5-ethyl-2-methoxybenzenesulfonamide hydrochloride (174)

To a stirred solution of compound 173 (0.11 g, 0.188 mmol) in DCM (4 mL) was added 4M HCl in dioxane (0.47 mL, 1.881 mmol) at 0° C. The reaction mixture was stirred at room temperature for 2 h. After completion (monitored by TLC), the mixture was concentrated under reduced pressure. The crude was triturated with Et₂O and dried under reduced pressure to afford title compound 174 (0.08 g, 69.74%) as a brown solid. LCMS calculated for C23H24N4O6S: 484.14; Found: 485.0 (M+1).

### Synthesis of methyl ((6-((3-((5-ethyl-2-methoxyphenyl)sulfonamido)-4-methoxybenzo[d]isoxazol-6-yl)oxy)pyridin-2-yl)methyl)carbamate (Syn. Ex. 106)

To a stirred solution of compound 174 (0.08 g, 0.153 mmol) in DCM (4 mL) was added NEt₃ (0.064 mL, 0.460 mmol) and methyl chloroformate (0.017 g, 0.184 mmol) at 0° C. The reaction mixture was stirred at 0° C. for 30 min. After completion (monitored by TLC), the mixture was quenched with ice-cold water and extracted with ethyl acetate. The combined organic layer was washed with brine, dried over anhydrous Na₂SO₄, filtered and concentrated under reduced pressure. The crude was purified by prep. HPLC to afford methyl ((6-((3-((5-ethyl-2-methoxyphenyl)sulfonamido)-4-methoxybenzo[d]isoxazol-6-yl)oxy)pyridin-2-yl)methyl)carbamate (Syn. Ex. 106; 20 mg, 23.98%) as a gummy solid. (See analytical data in Table 1).

### Examples 107-109

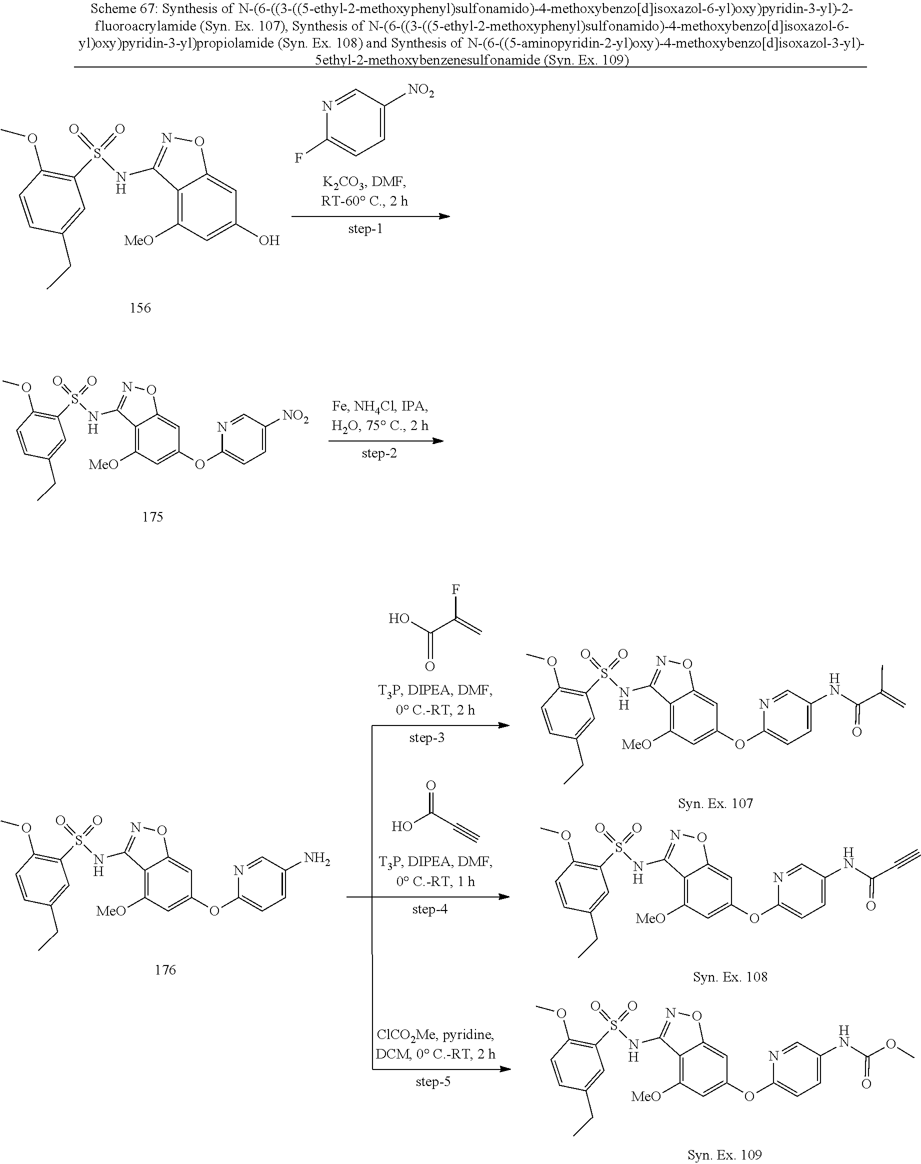

### Synthesis of 5-ethyl-2-methoxy-N-(4-methoxy-6-((5-nitropyridin-2-yl)oxy)benzo[d]isoxazol-3-yl)benzenesulfonamide (175)

To a stirred solution of compound 156 (0.20 g, 0.528 mmol) in DMF (3 mL) was added K₂CO₃ (0.14 g, 1.057 mmol) and 2-fluoro-5-nitropyridine (0.09 g, 0.634 mmol) at room temperature. The reaction mixture was stirred at 60° C. for 2 h. After completion (monitored by TLC), the mixture was quenched with ice-cold water, acidified with 1N HCl to adjust the pH 6-7 and extracted with EtOAc. The combined organic layer was washed with brine, dried over anhydrous Na₂SO₄, filtered and concentrated under reduced pressure. The crude was triturated with Et₂O to afford compound 175 (0.20 g, 67.29%) as a pale-yellow solid. LCMS calculated for C22H20N4O8S: 500.10; Found: 501.10 (M+1).

### Synthesis of N-(6-((5-aminopyridin-2-yl)oxy)-4-methoxybenzo[d]isoxazol-3-yl)-5-ethyl-2-methoxybenzenesulfonamide (176)

To a stirred solution of compound 175 (0.15 g, 0.299 mmol) in IPA (6 mL) and H₂O (3 mL) were added Fe (0.098 g, 1.498 mmol) and NH₄Cl (0.089 g, 1.498 mmol) at room temperature. The resulting mixture was stirred at 75° C. for 2 h. After completion (monitored by TLC), the mixture was filtered through a celite bed and filtrate was diluted with ice-cold water. The aqueous layer was extracted with EtOAc, dried over anhydrous Na₂SO₄, filtered and concentrated under reduced pressure to afford title compound 176 (0.12 g, 62.40%) as a brown-gummy solid. LCMS calculated for C22H22N4O6S: 470.13; Found: 471.10 (M+1).

### Synthesis of N-(6-((3-((5-ethyl-2-methoxyphenyl)sulfonamido)-4-methoxybenzo[d]isoxazol-6-yl)oxy)pyridin-3-yl)-2-fluoroacrylamide (Syn. Ex. 107)

To a stirred solution of compound 176 (0.08 g, 0.170 mmol) in DMF (8 mL) were added DIPEA (0.093 mL, 0.510 mmol) and 50% T₃P in EtOAc (0.32 g, 0.510 mmol). The mixture was stirred at 0° C. for 5 min followed by addition of 2-fluoroacrylic acid (0.018 g, 0.204 mmol). The mixture was stirred at room temperature for 2 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-(6-((3-((5-ethyl-2-methoxyphenyl)sulfonamido)-4-methoxybenzo[d]isoxazol-6-yl)oxy)pyridin-3-yl)-2-fluoroacrylamide (Syn. Ex. 107; 7.7 mg, 8.31%) as an off-white solid. (See analytical data in Table 1).

### Synthesis of N-(6-((3-((5-ethyl-2-methoxyphenyl)sulfonamido)-4-methoxybenzo[d]isoxazol-6-yl)oxy)pyridin-3-yl)propiolamide (Syn. Ex. 108)

To a stirred solution of compound 176 (0.08 g, 0.170 mmol) in DMF (3 mL) was added DIPEA (0.088 mL, 0.510 mmol), propiolic acid (0.01 g, 0.204 mmol) and 50% T₃P in EtOAc (0.32 mL, 0.510 mmol) at 0° C. The reaction mixture was stirred at room temperature for 1 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-(6-((3-((5-ethyl-2-methoxyphenyl)sulfonamido)-4-methoxybenzo[d]isoxazol-6-yl)oxy)pyridin-3-yl)propiolamide (Syn. Ex. 108; 3 mg, 3.34%) as an off-white solid. (See analytical data in Table 1).

### Synthesis of N-(6-((5-aminopyridin-2-yl)oxy)-4-methoxybenzo[d]isoxazol-3-yl)-5-ethyl-2-methoxybenzenesulfonamide (Syn. Ex. 109)

To a stirred solution of compound 176 (0.07 g, 0.148 mmol) in DCM (3 mL) was added pyridine (0.035 g, 0.446 mmol) and methyl chloroformate (0.017 g, 0.178 mmol) at ° C. The mixture was stirred at room temperature for 2 h. After completion (monitored by TLC), the mixture was quenched with ice cold water, acidified with 1N HCl (5 mL) and extracted with EtOAc. The combined organic layer was washed with brine, dried over anhydrous Na₂SO₄, filtered and concentrated under reduced pressure. The crude was purified by prep. HPLC to afford N-(6-((5-aminopyridin-2-yl)oxy)-4-methoxybenzo[d]isoxazol-3-yl)-5-ethyl-2-methoxybenzenesulfonamide (Syn. Ex. 109; 8 mg, 10.07%). (See analytical data in Table 1).

### Examples 110-111

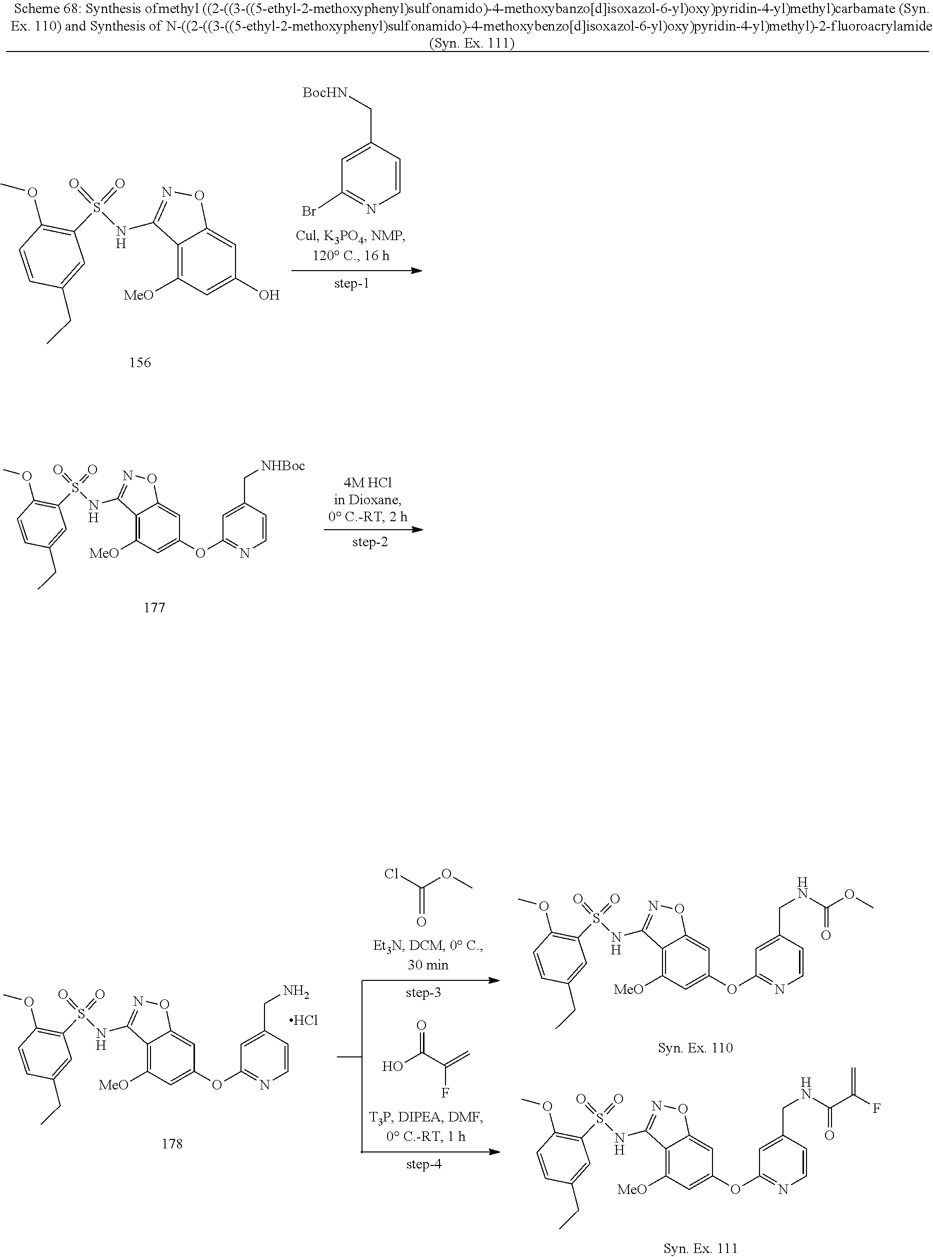

### Synthesis of tert-Butyl ((2-((3-((5-ethyl-2-methoxyphenyl)sulfonamido)-4-methoxybenzo[d]isoxazol-6-yl)oxy)pyridin-4-yl)methyl)carbamate (177)

To a stirred solution of compound 156 (0.20 g, 0.528 mmol) in NMP (3 mL) were added tert-Butyl ((2-bromopyridin-4-yl)methyl)carbamate (0.18 g, 0.634 mmol) and K₃PO₄ (0.35 g, 1.585 mmol). The reaction mixture was degassed with Argon for 10 min followed by addition of picolinic acid (0.013 g, 0.105 mmol) and CuI (0.003 g, 0.015 mmol). The reaction mixture was stirred at 120° C. for 16 h. After completion (monitored by TLC), the mixture was quenched with ice-cold water, acidified with 1N HCl and extracted with EtOAc. The combined organic layer was washed with brine, dried over anhydrous Na₂SO₄, filtered and concentrated under reduced pressure. The crude was purified by combi-flash chromatography using 54-65% EtOAc/Heptane to afford title compound 177 (0.21 g, 65.37%) as a brown solid. LCMS calculated for C28H32N4O8S: 584.19; Found: 585.30 (M+1).

### Synthesis of N-(6-((4-(aminomethyl)pyridin-2-yl)oxy)-4-methoxybenzo[d]isoxazol-3-yl)-5-ethyl-2-methoxybenzenesulfonamide hydrochloride (178)

To a stirred solution of compound 177 (0.21 g, 0.359 mmol) in DCM (5 mL) was added 4M HCl in Dioxane (0.89 mL, 3.591 mmol) at 0° C. The reaction mixture was stirred at room temperature for 2 h. After completion (monitored by TLC), the mixture was concentrated under reduced pressure. The crude was triturated with Et₂O to afford title compound 178 (0.16 g, 79.44%) as a brown solid. LCMS calculated for C23H24N4O6S: 484.14; Found: 485.20 (M+1).

### Synthesis of methyl ((2-((3-((5-ethyl-2-methoxyphenyl)sulfonamido)-4-methoxybenzo[d]isoxazol-6-yl)oxy)pyridin-4-yl)methyl)carbamate (Syn. Ex. 110)

To a stirred solution of compound 178 (0.08 g, 0.153 mmol) in DCM (3 mL) were added NEt₃ (0.067 mL, 0.484 mmol) and methyl chloroformate (0.017 g, 0.184 mmol) at 0° C., while maintaining the pH 7-8. The reaction mixture was stirred at 0° C. for 30 min. After completion (monitored by TLC), the mixture was quenched with ice-cold water and extracted with EtOAc. The combined organic layer was washed with brine, dried over anhydrous Na₂SO₄, filtered and concentrated under reduced pressure. The crude was purified by prep. HPLC to afford methyl ((2-((3-((5-ethyl-2-methoxyphenyl)sulfonamido)-4-methoxybenzo[d]isoxazol-6-yl)oxy)pyridin-4-yl)methyl)carbamate (Syn. Ex. 110; 24 mg, 28.53%) as a gummy solid. (See analytical data in Table 1).

### Synthesis of N-((2-((3-((5-ethyl-2-methoxyphenyl)sulfonamido)-4-methoxybenzo[d]isoxazol-6-yl)oxy)pyridin-4-yl)methyl)-2-fluoroacrylamide (Syn. Ex. 111)

To a stirred solution of compound 178 (0.07 g, 0.134 mmol) in DMF (3 mL) was added DIPEA (0.07 mL, 0.411 mmol), 2-fluoroacrylic acid (0.014 g, 0.161 mmol) and 50% T₃P in EtOAc (0.25 mL, 0.403 mmol) at 0° C. The reaction mixture was stirred at room temperature for 1 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-((2-((3-((5-ethyl-2-methoxyphenyl)sulfonamido)-4-methoxybenzo[d]isoxazol-6-yl)oxy)pyridin-4-yl)methyl)-2-fluoroacrylamide (Syn. Ex. 111; 18 mg, 23.72%) as an off-white solid. (See analytical data in Table 1).

### Example 112

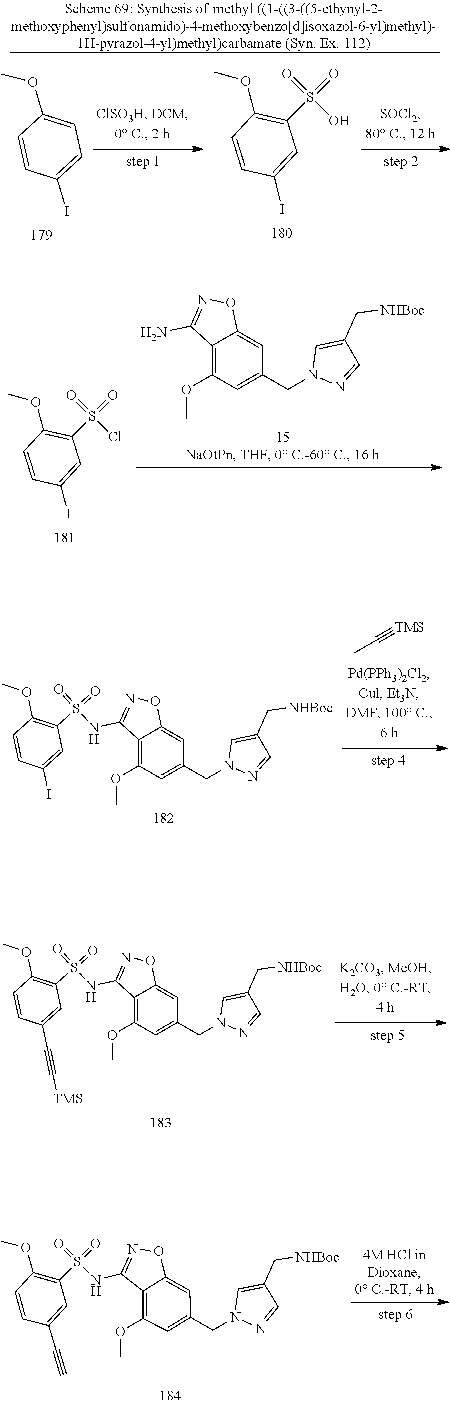

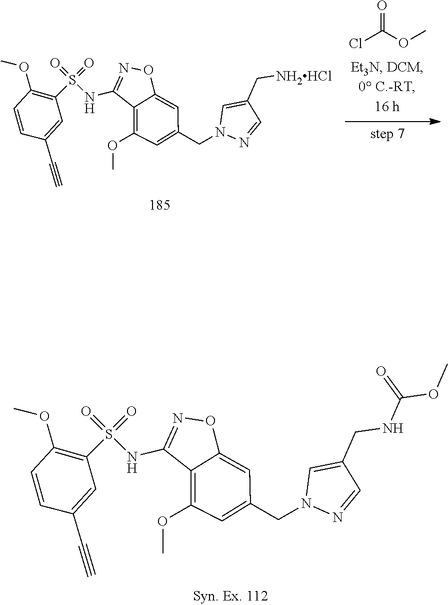

### Synthesis of 5-iodo-2-methoxybenzenesulfonic acid (180)

To a stirred solution of compound 179 (20 g, 85.455 mmol) in DCM (200 mL) was added ClSO₃H (18.26 mL, 256.366 mmol) at 0° C. The reaction mixture was stirred at 0° C. for 2 h. After completion (monitored by TLC), the mixture was quenched with saturated NaHCO₃ and extracted with DCM. The combined organic layer was dried over anhydrous Na₂SO₄ and concentrated under reduced pressure to afford title compound 180 (5.80 g, 9.29%) as a brown solid. LCMS calculated for C7H7IO4S: 313.91; Found: 312.90 (M−1).

### Synthesis of 5-iodo-2-methoxybenzenesulfonyl chloride (181)

To a stirred solution of compound 180 (4.10 g, 13.053 mmol) was added SOCl₂ (2.86 mL, 39.160 mmol) at 0° C. The reaction mixture was stirred at 80° C. for 12 h. After completion (monitored by TLC), the mixture was concentrated under reduced pressure. The crude was purified by combi-flash chromatography using 15-17% EtOAc/Heptane to afford title compound 181. The crude was carried forward to the next reaction.

### Synthesis of tert-Butyl ((1-((3-((5-iodo-2-methoxyphenyl)sulfonamido)-4-methoxybenzo[d]isoxazol-6-yl)methyl)-1H-pyrazol-4-yl)methyl)carbamate (182)

To a stirred solution of compound 15 (0.50 g, 1.353 mmol) in THF (15 mL) was added compound 181 (1.50 g, 4.510 mmol) followed by sodium tert-pentoxide (1.56 g, 13.532 mmol) at 0° C. The reaction mixture was stirred at 60° C. for 16 h. After completion (monitored by TLC), the mixture was quenched with ice-cold water and extracted with EtOAc. The combined organic layer was washed with brine, dried over anhydrous Na₂SO₄, filtered and concentrated under reduced pressure to afford title compound 182 (0.60 g, 10.72%) as a brown solid. LCMS calculated for C25H28IN5O7S: 669.08; Found: 670.10 (M+1).

### Synthesis of tert-Butyl ((1-((4-methoxy-3-((2-methoxy-5-((trimethylsilyl)ethynyl)phenyl)sulfonamido)benzo[d]isoxazol-6-yl)methyl)-1H-pyrazol-4-yl)methyl)carbamate (183)

To a stirred solution of compound 182 (1 g, 1.493 mmol) in THF (20 mL) was added DIPEA (0.82 mL, 4.481 mmol). The reaction was degassed with argon purging for 10 min. followed by addition of CuI (0.023 g, 0.119 mmol), ethynyltrimethylsilane (0.44 g, 4.481 mmol) and Pd(PPh₃)₂Cl₂ (0.055 g, 0.074 mmol). The reaction mixture was stirred at 100° C. for 6 h. After completion (monitored by TLC), the mixture was cooled at room temperature, filtered through a celite pad, washed with EtOAc and concentrated under reduced pressure. The crude was purified by reverse phase column chromatography using 20-30% ACN/H₂O to afford title compound 183 (0.20 g, 8.18%) as a brown solid. LCMS calculated for C30H37N507SSi: 639.22; Found: 638.30 (M−1).

### Synthesis of tert-Butyl ((1-((3-((5-ethynyl-2-methoxyphenyl)sulfonamido)-4-methoxybenzo[d]isoxazol-6-yl)methyl)-1H-pyrazol-4-yl)methyl)carbamate (184)

To a stirred solution of compound 183 (0.03 g, 0.046 mmol) in MeOH (10 mL) was added K₂CO₃ (0.013 g, 0.056 mmol) followed by the addition of H₂O (2 mL) at 0° C. The resulting mixture was stirred at room temperature for 4 h. After completion (monitored by TLC), the mixture was concentrated under reduced pressure. The crude was quenched with water, extracted with DCM and dried over Na₂SO₄. The DCM layer was dried under reduced pressure to afford title compound 184 (0.01 g, 31.74%) as an off-white solid. LCMS calculated for C27H29N5O7S: 567.18; Found: 568.20 (M+1).

### Synthesis of N-(6-((4-(aminomethyl)-1H-pyrazol-1-yl)methyl)-4-methoxybenzo[d]isoxazol-3-yl)-5-ethynyl-2-methoxybenzenesulfonamide hydrochloride (185)

To a stirred solution of compound 184 (10 mg, 0.017 mmol) in DCM (3 mL) was added 4M HCl in Dioxane (1 mL) at 0° C. The reaction mixture was at room temperature for 4 h. After completion (monitored by TLC), the mixture was concentrated under reduced pressure. The crude was triturated with Et₂O and dried under reduced pressure to afford title compound 185 (6 mg, 28.73%) as a brown solid. LCMS calculated for C22H21N5O5S: 467.13; Found: 468.30 (M+1).

### Synthesis of methyl ((1-((3-((5-ethynyl-2-methoxyphenyl)sulfonamido)-4-methoxybenzo[d]isoxazol-6-yl)methyl)-1H-pyrazol-4-yl)methyl)carbamate (Syn. Ex. 112)

To a stirred solution of compound 185 (6 mg, 0.011 mmol) in DCM (5 mL) were added Et₃N (0.0052 mL, 0.035 mmol) and methyl chloroformate (1.3 mg, 0.014 mmol) at 0° C. The reaction mixture was stirred at room temperature for 16 h. After completion (monitored by TLC), the mixture was quenched with ice-cold water and extracted with DCM. The combined organic layer was dried over anhydrous Na₂SO₄, filtered and concentrated under reduced pressure. The crude was purified by prep. HPLC to afford methyl((1-((3-((5-ethynyl-2-methoxyphenyl)sulfonamido)-4-methoxybenzo[d]isoxazol-6-yl)methyl)-1H-pyrazol-4-yl)methyl)carbamate (Syn. Ex. 112; 2 mg, 31.25%) as an off-white solid. (See analytical data in Table 1).

### Example 113

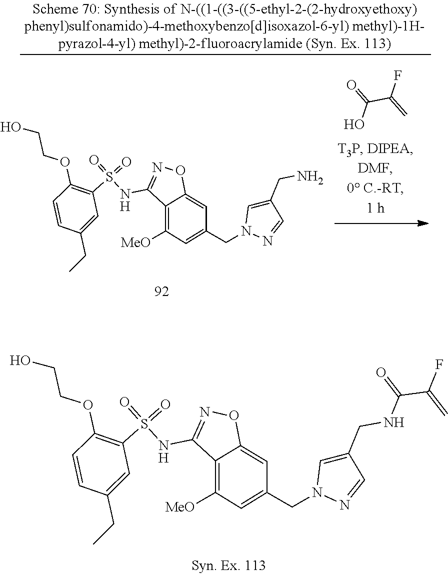

To a stirred solution of compound 92 (80 mg, 0.159 mmol) in DMF (2 mL) was added DIPEA (0.083 mL, 0.477 mmol), 2-fluoroprop-2-enoic acid (28 mg, 0.318 mmol) and T₃P (50%) in EtOAc (0.30 mL, 0.477 mmol) at 0° C. The reaction mixture was stirred at room temperature for 1 h. After completion (monitored by TLC), the mixture was quenched with ice-cold water and extracted with EtOAc. The combined organic layer was dried over anhydrous Na₂SO₄ and concentrated under reduced pressure. The crude was purified by prep. HPLC to afford N-((1-((3-((5-ethyl-2-(2-hydroxyethoxy)phenyl)sulfonamido)-4-methoxybenzo[d]isoxazol-6-yl)methyl)-1H-pyrazol-4-yl)methyl)-2-fluoroacrylamide (Syn. Ex. 113; 10 mg, 10.98%) as an off-white solid. (See analytical data in Table 1).

### Example 114

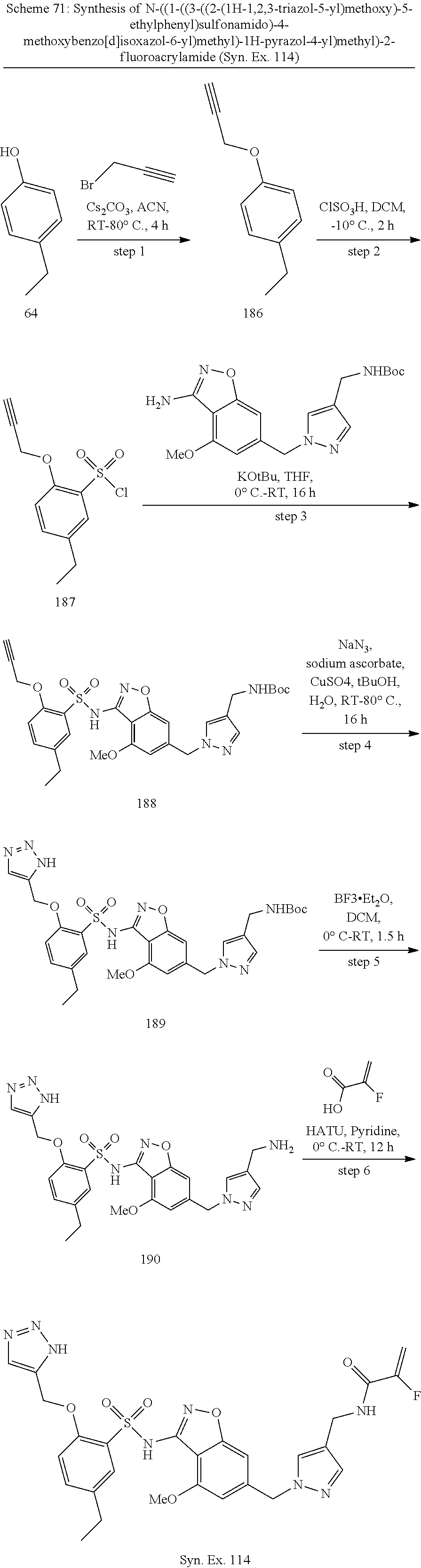

### Synthesis of 1-ethyl-4-(prop-2-yn-1-yloxy)benzene (186)

To a stirred solution of 4-ethylphenol 64 (6 g, 49.111 mmol) in ACN (80 mL) were added Cs₂CO₃ (32 g, 98.213 mmol) and 3-bromoprop-1-yne (5.54 g, 73.667 mmol) at room temperature. The reaction mixture was stirred at 80° C. for 4 h. After completion (monitored by TLC), the mixture was filtered through a celite bed and concentrated under reduced pressure. The crude was purified by combi-flash column chromatography using 25% EtOAc/Heptane to afford title compound 186 (8 g, 86.41%) as a pale-yellow liquid. LCMS calculated for C11H12O: 160.09; Found: 161.20 (M+1).

### Synthesis of 5-ethyl-2-(prop-2-yn-1-yloxy)benzenesulfonyl chloride (187)

To a solution of 1-ethyl-4-(prop-2-yn-1-yloxy)benzene 186 (6 g, 37.448 mmol) in DCM (60 mL) was added ClSO₃H (5.03 mL, 75.653 mmol) at −10° C. The reaction mixture was stirred at −10° C. for 2 h. After completion (monitored by TLC), the mixture was quenched with ice-cold water and extracted with EtOAc. The combined organic layer was dried over anhydrous Na₂SO₄, filtered and concentrated under reduced pressure. The crude was purified by combi-flash chromatography using 5-10% EtOAc/Heptane to afford title compound 187 (0.80 g, 4.95%) as a pale-yellow sticky solid. ¹H NMR (400 MHz, DMSO-d₆) δ ppm 7.55 (br s, 1H), 7.13 (br d, J=8.29 Hz, 1H), 6.96 (br d, J=7.46 Hz, 1H), 4.74 (br s, 2 H), 2.44-2.58 (m, 3H), 1.14 (br t, J=7.05 Hz, 3H).

### Synthesis of tert-Butyl ((1-((3-((5-ethyl-2-(prop-2-yn-1-yloxy)phenyl)sulfonamido)-4-methoxybenzo[d]isoxazol-6-yl)methyl)-1H-pyrazol-4-yl)methyl)carbamate (188)

To a stirred solution of compound 15 (0.40 g, 1.071 mmol) in THF (20 mL) were added KOtBu 1M in THF (1.60 mL, 1.606 mmol) and compound 187 (0.41 g, 1.606 mmol) at 0° C. The reaction mixture was stirred at room temperature for 16 h. After completion (monitored by TLC), the mixture was quenched with saturated citric acid and extracted with EtOAc. The combined organic layer was washed with brine, dried over anhydrous Na₂SO₄ and concentrated under reduced pressure. The crude was purified by combi-flash chromatography using 90% EtOAc/Heptane to afford title compound 188 (0.30 g, 21.62%) as an off-white solid. LCMS calculated for C29H33N5O7S: 595.21; Found: 594.30 (M−1).

### Synthesis of tert-Butyl ((1-((3-((2-((1H-1,2,3-triazol-5-yl)methoxy)-5-ethylphenyl)sulfonamido)-4-methoxybenzo[d]isoxazol-6-yl)methyl)-1H-pyrazol-4-yl)methyl)carbamate (189)

To a stirred solution of compound 188 (0.30 g, 0.503 mmol) in tBuOH (10 mL) and water (5 mL) were added NaN₃ (0.032 g, 0.503 mmol) and (+) sodium L-ascorbate (0.0099 g, 0.050 mmol) in water (1 mL) followed by the addition of CuSO₄ (0.7 mg, 0.005 mmol) in water (1 mL) at room temperature. The reaction mixture was stirred at 80° C. for 16 h. After completion (monitored by TLC), the mixture was filtered through celite bed, washed with 10% MeOH in DCM and dried under reduced pressure to afford title compound 189 (0.25 g, 55.65%) as a yellow solid. LCMS calculated for C29H34N807S: 638.23; Found: 639.20 (M+1).

### Synthesis of 2-((1H-1,2,3-triazol-5-yl)methoxy)-N-(6-((4-(aminomethyl)-1H-pyrazol-1-yl)methyl)-4-methoxybenzo[d]isoxazol-3-yl)-5-ethylbenzenesulfonamide (190)

To a stirred solution of compound 189 (0.20 g, 0.313 mmol) in DCM (10 mL) was added BF₃·Et₂O (0.50 mL, 1.982 mmol) at 0° C. The reaction mixture was stirred at room temperature for 1.5 h. After completion (monitored by TLC), the mixture was concentrated under reduced pressure. The residue was washed with Et₂O (10 mL) and dried under reduced pressure to afford title compound 190 (0.18 g, 90.09%) as a pale-yellow solid. LCMS calculated for C24H26N805S: 538.17; Found: 539.20 (M+1).

### Synthesis of N-((1-((3-((2-((1H-1,2,3-triazol-5-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 114)

To a stirred solution of compound 190 (0.15 g, 0.278 mmol) in pyridine (5 mL) were added 2-fluoroacrylic acid (0.03 g, 0.417 mmol) and HATU (0.31 g, 0.835 mmol) at 0° C. The reaction mixture was stirred at room temperature for 12 h. After completion (monitored by TLC), the mixture was diluted with water and extracted with EtOAc. The combined organic layer was dried over anhydrous Na₂SO₄, filtered and concentrated under reduced pressure. The crude was purified by prep. HPLC to afford N-((1-((3-((2-((1H-1,2,3-triazol-5-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 114; 6 mg, 3.43%) as an off-white solid. (See analytical data in Table 1).

### Example 115-117

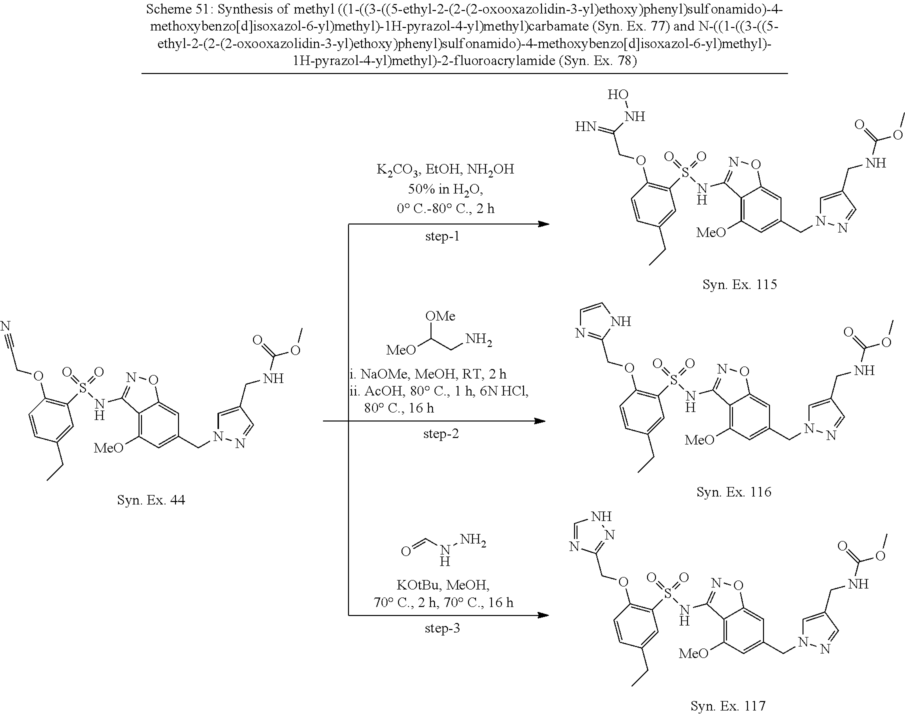

### Synthesis of methyl ((1-((3-((5-ethyl-2-(2-(hydroxyamino)-2-iminoethoxy)phenyl)sulfonamido)-4-methoxybenzo[d]isoxazol-6-yl)methyl)-1H-pyrazol-4-yl)methyl)carbamate (Syn. Ex. 115)

To a stirred solution of Syn. Ex. 44 (0.12 g, 0.216 mmol) in EtOH (5 mL) were added K₂CO₃ (0.04 g, 0.324 mmol) and 50% NH₂OH in H₂O (0.02 mL, 0.432 mmol) at 0° C. The reaction mixture was stirred at 80° C. for 2 h. After completion (monitored by TLC), the mixture was concentrated under reduced pressure. The crude was purified by prep. HPLC to afford methyl ((1-((3-((5-ethyl-2-(2-(hydroxyamino)-2-iminoethoxy)phenyl)sulfonamido)-4-methoxybenzo[d]isoxazol-6-yl)methyl)-1H-pyrazol-4-yl)methyl)carbamate (Syn. Ex. 115; 15 mg, 11.44%) as an off-white solid. (See analytical data in Table 1).

### Synthesis of methyl ((1-((3-((2-((1H-imidazol-2-yl)methoxy)-5-ethylphenyl)sulfonamido)-4-methoxybenzo[d]isoxazol-6-yl)methyl)-1H-pyrazol-4-yl)methyl)carbamate (Syn. Ex. 116)

To a stirred solution of Syn. Ex. 44 (0.16 g, 0.288 mmol) in MeOH (5 mL) was added NaOMe (0.04 g, 0.865 mmol) at room temperature. The reaction mixture was stirred at 70° C. for 2 h followed by addition of 2,2-diethoxyethan-1-amine (0.04 g, 0.346 mmol) and AcOH (0.03 mL, 0.577 mmol). The reaction mixture was stirred at 80° C. for 1 h, cooled to room temperature followed by addition of 6N HCl (0.10 mL) and stirred at 80° C. for 16 h. After completion (monitored by TLC), the mixture was concentrated under reduced pressure. The residue was dissolved in ice-cold water and extracted with 10% MeOH in DCM. The combined organic layer was washed with brine, dried over anhydrous Na₂SO₄, filtered and concentrated under reduced pressure. The crude was purified by prep. HPLC to afford methyl ((1-((3-((2-((1H-imidazol-2-yl)methoxy)-5-ethylphenyl)sulfonamido)-4-methoxybenzo[d]isoxazol-6-yl)methyl)-1H-pyrazol-4-yl)methyl)carbamate (Syn. Ex. 116; 22.5 mg, 12.84%) as an off-white solid. (See analytical data in Table 1).

### Synthesis of methyl ((1-((3-((2-((1H-1,2,4-triazol-3-yl)methoxy)-5-ethylphenyl)sulfonamido)-4-methoxybenzo[d]isoxazol-6-yl)methyl)-1H-pyrazol-4-yl)methyl)carbamate (Syn. Ex. 117)

To a stirred solution of Syn. Ex. 44 (0.06 g, 0.108 mmol) in MeOH (2 mL) was added KOtBu (0.01 g, 0.129 mmol). The reaction mixture was stirred at 70° C. for 2 h, then cooled to room temperature followed by addition of formyl hydrazide (0.03 g, 0.540 mmol). The reaction mixture was stirred at 70° C. for 16 h. After completion (monitored by TLC), the mixture was concentrated under reduced pressure. The crude was purified by prep. HPLC to afford methyl((1-((3-((2-((1H-1,2,4-triazol-3-yl)methoxy)-5-ethylphenyl)sulfonamido)-4-methoxybenzo[d]isoxazol-6-yl)methyl)-1H-pyrazol-4-yl)methyl)carbamate (Syn. Ex. 117; 13 mg, 19.51%) as an off-white solid. (See analytical data in Table 1).

### Example 118

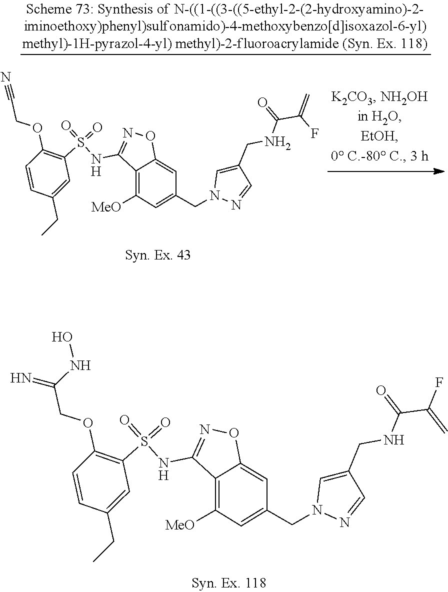 [SMILES: C=C(F)C(=O)NCc1cnn(Cc2cc(OC)c3c(NS(=O)(=O)c4cc(CC)ccc4OCC#N)noc3c2)c1 ; C=C(F)C(=O)NCc1cnn(Cc2cc(OC)c3c(NS(=O)(=O)c4cc(CC)ccc4OCC(=N)NO)noc3c2)c1]

To a stirred solution of Syn. Ex. 43 (0.16 g, 0.281 mmol) in EtOH (5 mL) were added K₂CO₃ (0.05 g, 0.421 mmol) and 50% NH₂OH in H₂O (0.03 g, 0.562 mmol) at 0° C. The reaction mixture was stirred at 80° C. for 3 h. After completion (monitored by TLC), the mixture was quenched with ice-cold water and extracted with 10% MeOH in DCM. The combined organic layer was washed with brine, dried over anhydrous Na₂SO₄, filtered and concentrated under reduced pressure. The crude was purified by prep. HPLC to afford N-((1-((3-((5-ethyl-2-(2-(hydroxyamino)-2-iminoethoxy)phenyl)sulfonamido)-4-methoxybenzo[d]isoxazol-6-yl)methyl)-1H-pyrazol-4-yl)methyl)-2-fluoroacrylamide (Syn. Ex. 118; 11 mg, 6.34%) as an off-white solid. (See analytical data in Table 1).

### Example 119

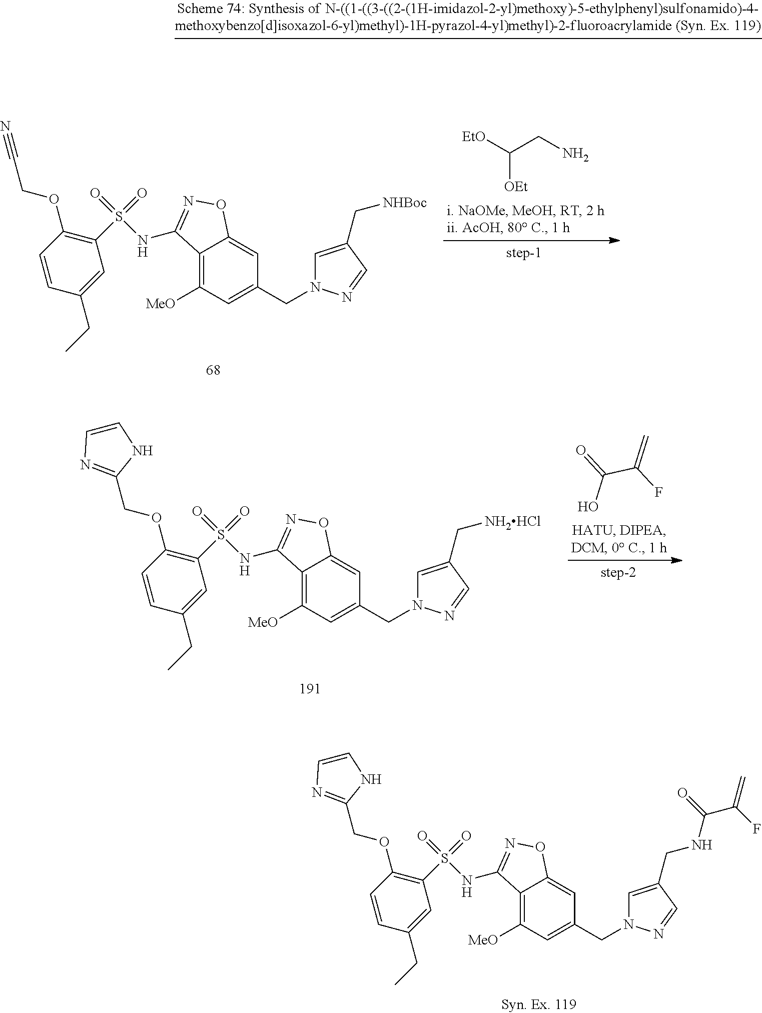

### Synthesis of 2-((1H-imidazol-2-yl)methoxy)-N-(6-((4-(aminomethyl)-1H-pyrazol-1-yl)methyl)-4-methoxybenzo[d]isoxazol-3-yl)-5-ethylbenzenesulfonamide hydrochloride (191)

To a stirred solution of compound 68 (0.25 g, 0.418 mmol) in MeOH (5 mL) was added NaOMe (0.08 mL, 1.257 mmol) at room temperature. The reaction mixture was stirred at the same temperature for 2 h. followed by addition of 2,2-diethoxyethan-1-amine (0.09 mL, 0.628 mmol) and AcOH (0.04 mL, 0.838 mmol). The reaction mixture was stirred at 80° C. for 1 h, cooled to room temperature followed by addition of 6N HCl. The reaction mixture was stirred at 80° C. for 16 h. After completion (monitored by TLC), the mixture was concentrated under reduced pressure, washed with Et₂O and dried under reduced pressure to afford title compound 191 (0.22 g, 76.57%) as a pale-yellow solid. LCMS calculated for C25H27N705S: 537.18; Found: 536.20 (M−1).

### Synthesis of N-((1-((3-((2-((1H-imidazol-2-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 119)

To a stirred solution of 2-fluoroacrylic acid (0.04 g, 0.522 mmol) in DCM (5 mL) were added DIPEA (0.22 mL, 1.306 mmol) and HATU (0.15 g, 0.391 mmol) followed by the addition of compound 191 (0.15 g, 0.261 mmol) at 0° C. The reaction mixture was stirred at room temperature for 1 h. After completion (monitored by TLC), the mixture was quenched with ice-cold water and extracted with 10% MeOH in DCM. The combined organic layer was dried over anhydrous Na₂SO₄ and concentrated under reduced pressure. The crude was purified by prep. HPLC to afford N-((1-((3-((2-((1H-imidazol-2-yl)methoxy)-5-ethylphenyl)sulfonamido)-4-methoxybenzo[d]isoxazol-6-yl)methyl)-1H-pyrazol-4-yl)methyl)-2-fluoroacrylamide (Syn. Ex. 119; 8 mg, 4.96%) as an off-white solid. (See analytical data in Table 1).

### Example 120

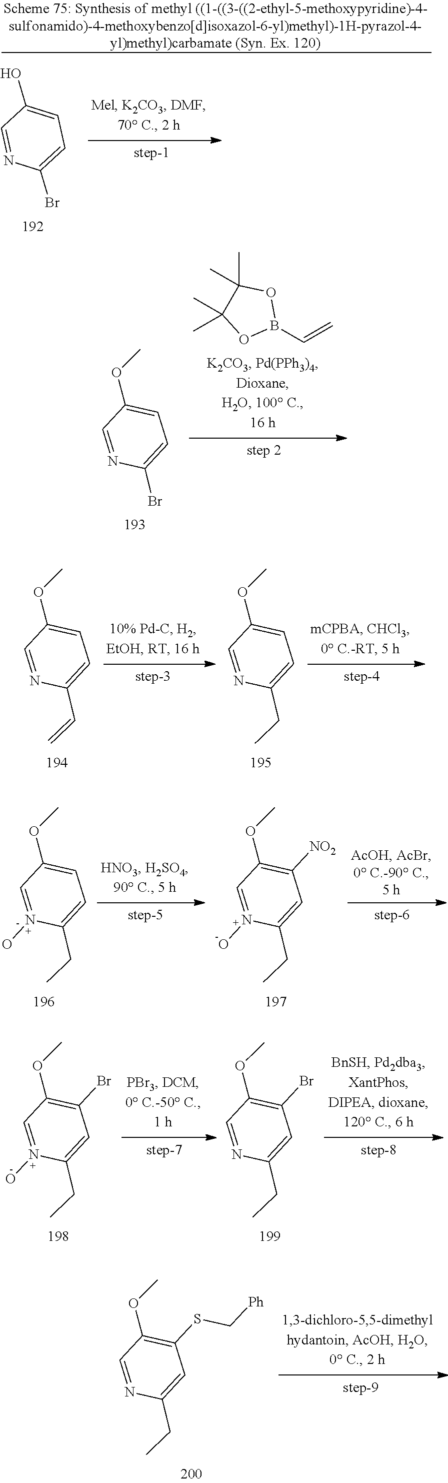

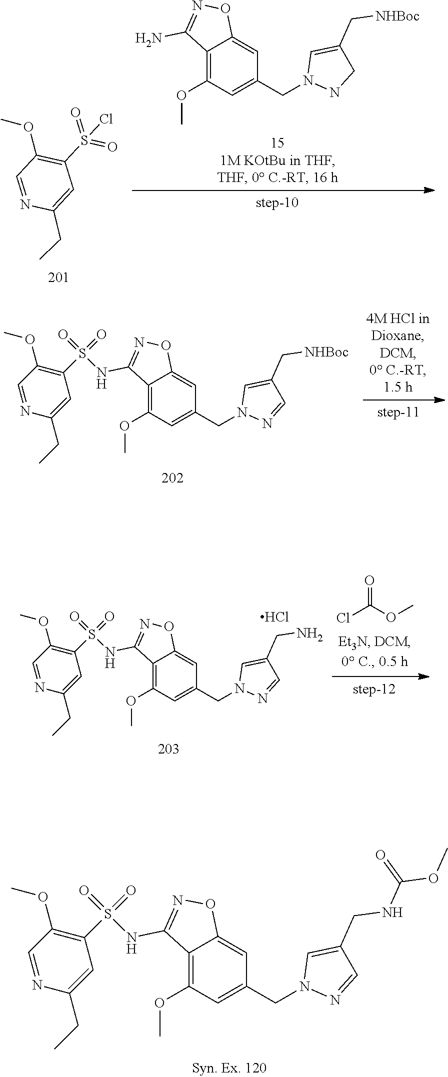

### Synthesis of 2-bromo-5-methoxypyridine (193)

To a stirred solution of compound 192 (15 g, 86.206 mmol) in DMF (150 mL) were added K₂CO₃ (35.74 g, 258.620 mmol) and Mel (6.44 mL, 103.448 mmol). The reaction mixture was stirred at 70° C. for 2 h. After completion (monitored by TLC), the mixture was quenched with water and extracted with Et₂O. The combined organic layer was dried over anhydrous Na₂SO₄ and concentrated under reduced pressure to afford title compound 193 (10 g, 56.75%) as a pale-yellow oil. LCMS calculated for C6H6BrNO: 186.96; Found: 188 (M+2).

### Synthesis of 5-methoxy-2-vinylpyridine (194)

To a stirred solution of compound 193 (10 g, 53.185 mmol) in 1,4-Dioxane (100 mL) were added K₂CO₃ (22.05 g, 159.557 mmol) and 4,4,5,5-tetramethyl-2-vinyl-1,3,2-dioxaborolane (9.82 g, 63.822 mmol) and water (20 mL). The reaction mixture was degassed with Argon for 3 min followed by addition of Pd(PPh₃)₄ (3.16 g, 2.659 mmol). The reaction mixture was stirred at 100° C. for 16 h. After completion (monitored by TLC), the mixture was filtered through celite bed, washed with EtOAc and concentrated under reduced pressure. The crude was purified by combi-flash column chromatography using 30% EtOAc/Heptane to afford title compound 194 (8 g, 85.90%) as a pale-yellow liquid. LCMS calculated for C8H9NO: 135.07; Found; 136.20 (M+1).

### Synthesis of 2-ethyl-5-methoxypyridine (195)

To a stirred solution of compound 194 (8 g, 59.184 mmol) in EtOH (100 mL) was added 10% Pd—C(75.58 g, 71.021 mmol). The reaction mixture was stirred at room temperature for 16 h under H₂ atmosphere. After completion (monitored by TLC), the reaction mixture was filtered through a celite bed and concentrated under reduced pressure to afford title compound 195 (10 g, 83.13%) as a brown oil. ¹H NMR (400 MHz, CHLOROFORM-d) δ ppm 8.22 (br s, 1H), 7.04-7.10 (m, 2H), 3.83 (s, 3H), 2.77 (q, J=7.46 Hz, 2H), 1.28 (br t, J=7.46 Hz, 3H).

### Synthesis of 2-ethyl-5-methoxypyridine 1-oxide (196)

To a stirred solution of compound 195 (8 g, 58.317 mmol) in CHCl₃ (200 mL) was added mCPBA (15.88 g, 87.476 mmol) at 0° C. The reaction mixture was stirred at room temperature for 5 h. After completion (monitored by TLC), the mixture was quenched with saturated NaHCO₃ and extracted with DCM. The combined organic layer was dried over anhydrous Na₂SO₄ and concentrated under reduced pressure to afford title compound 196 (7 g, 51.71%) as a pale-yellow solid. LCMS calculated for C8H11NO2: 153.08; Found: 154 (M+1).

### Synthesis of 2-ethyl-5-methoxy-4-nitropyridine 1-oxide (197)

To a stirred solution of compound 196 (7 g, 45.697 mmol) in H₂SO₄ (20 mL) was added HNO₃ (16 mL). The reaction mixture was stirred at 90° C. for 5 h under N₂ atmosphere. After completion (monitored by TLC), the mixture was quenched with ice-cold water (100 mL), basified with 1N NaOH and extracted with EtOAc. The combined organic layer was dried over anhydrous Na₂SO₄ and concentrated under reduced pressure to afford a title compound 197 (3 g, 32.39%) as a yellow oil. LCMS calculated for C8H10N2O4: 198.06; Found: 198.80 (M+1).

### Synthesis of 4-bromo-2-ethyl-5-methoxypyridine 1-oxide (198)

To a stirred solution of mixture of compound 197 (4.50 g, 22.706 mmol) in AcOH (30 mL) was added AcBr (20 mL) at 0° C. The reaction mixture was stirred at 90° C. for 5 h. After completion (monitored by TLC), the mixture was quenched with ice-cold water (60 mL), basified with 2N NaOH and extracted with DCM. The combined organic layer was dried over anhydrous Na₂SO₄ and concentrated under reduced pressure to afford title compound 198 (4 g, 74.15%) as a pale-yellow solid. LCMS calculated for C8H10BrNO2: 230.99; Found: 232 (M+2).

### Synthesis of 4-bromo-2-ethyl-5-methoxypyridine (199)

To a stirred solution of compound 198 (4 g, 17.235 mmol) in DCM (50 mL) was added PBr₃ (10 mL) at 0° C. The reaction mixture was stirred at 50° C. for 1 h. After completion (monitored by TLC), the mixture was quenched with ice-cold water, basified with 2N NaOH and extracted with DCM. The combined organic layer was dried over anhydrous Na₂SO₄ and concentrated under reduced pressure to afford title compound 199 (3.50 g, 87.87%) as a pale-yellow oil. LCMS calculated for C8H10BrNO: 214.99; Found: 216 (M+2).

### Synthesis of 4-(benzylthio)-2-ethyl-5-methoxypyridine (200)

To a stirred solution of compound 199 (3.50 g, 16.197 mmol) in 1,4-Dioxane (100 mL) were added BnSH (2.01 g, 16.197 mmol) and DIPEA (8.63 mL, 48.593 mmol) followed by XantPhos (0.77 g, 1.295 mmol). The reaction mixture was degassed with Argon for 10 min. followed by addition of Pd₂(dba)₃ (0.76 g, 0.809 mmol). The reaction mixture was stirred at 120° C. for 6 h. After completion (monitored by TLC), the mixture was quenched with ice-cold water and extracted with EtOAc. The combined organic layer was washed with brine, dried over anhydrous Na₂SO₄, filtered and concentrated under reduced pressure. The crude was purified by combi-flash column chromatography using 10-15% EtOAc/Heptane to afford title compound 200 (3 g, 71.19%) as a pale-yellow solid. LCMS calculated for C15H17NOS: 259.10; Found: 260.10 (M+1).

### Synthesis of 2-ethyl-5-methoxypyridine-4-sulfonyl chloride (201)

To a stirred solution of compound 200 (0.50 g, 1.927 mmol) in AcOH (5 mL) and H2O (1 mL) was added 1,3-dichloro-5,5-dimethylhydantoin (0.77 g, 3.855 mmol) at 0° C. The reaction mixture was stirred at 0° C. for 2 h. After completion (monitored by TLC), the mixture was quenched with ice-cold water and extracted with EtOAc. The combined organic layer was washed with saturated NaHCO₃, dried over anhydrous Na₂SO₄, filtered and concentrated under reduced pressure. The crude was purified by combi-flash column chromatography using 10% EtOAc/Heptane to afford title compound 201 (0.37 g, 44.79%) as a colourless sticky solid. LCMS calculated for C8H10ClNO3S: 235.01; Found: 236 (M+1).

### Synthesis of tert-Butyl ((1-((3-((2-ethyl-5-methoxypyridine)-4-sulfonamido)-4-methoxybenzo[d]isoxazol-6-yl)methyl)-1H-pyrazol-4-yl)methyl)carbamate (202)

To a stirred solution of compound 15 (0.30 g, 0.803 mmol) in THF (20 mL) were added 1M KOtBu in THF (2.41 mL, 2.410 mmol) and compound 201 (0.28 g, 1.205 mmol) at 0° C. The reaction mixture was stirred at room temperature for 16 h. After completion (monitored by TLC), the mixture was quenched with saturated citric acid and extracted with EtOAc. The combined organic layer was washed with brine, dried over anhydrous Na₂SO₄ and concentrated under reduced pressure. The crude was washed with Et₂O to afford title compound 202 (0.27 g, 27.58%) as a pale-yellow solid. LCMS calculated for C26H32N6O7S: 572.21; Found: 571.20 (M−1).

### Synthesis of N-(6-((4-(aminomethyl)-1H-pyrazol-1-yl)methyl)-4-methoxybenzo[d]isoxazol-3-yl)-2-ethyl-5-methoxypyridine-4-sulfonamide hydrochloride (203)

To a stirred solution of compound 202 (0.26 g, 0.454 mmol) in DCM (mL) was added 4M HCl in Dioxane (9.90 mL, 39.628 mmol) at 0° C. The reaction mixture was stirred at room temperature for 1.5 h. After completion (monitored by TLC), the mixture was concentrated under reduced pressure. The crude was washed with Et₂O and concentrated under reduced pressure to afford title compound 203 (0.23 g, 43.20%) as a pale-yellow solid. LCMS calculated for C21H24N6O5S: 472.15; Found: 471.10 (M−1).

### Synthesis of methyl ((1-((3-((2-ethyl-5-methoxypyridine)-4-sulfonamido)-4-methoxybenzo[d]isoxazol-6-yl)methyl)-1H-pyrazol-4-yl)methyl)carbamate (Syn. Ex. 120)

To a solution of compound 203 (0.20 g, 0.392 mmol) in DCM (20 mL) were added Et₃N (0.16 mL, 1.179 mmol) and methyl chloroformate (0.04 g, 0.471 mmol) at 0° C. The reaction mixture was stirred at 0° C. for 30 min. After completion (monitored by TLC), the mixture was quenched with water and extracted with EtOAc. The combined organic layer was dried over anhydrous Na₂SO₄, filtered and concentrated under reduced pressure. The crude was purified by prep. HPLC to afford methyl ((1-((3-((2-ethyl-5-methoxypyridine)-4-sulfonamido)-4-methoxybenzo[d]isoxazol-6-yl)methyl)-1H-pyrazol-4-yl)methyl)carbamate (Syn. Ex. 120; 20 mg, 9.20%) as an off-white solid. (See analytical data in Table 1).

### Example 121

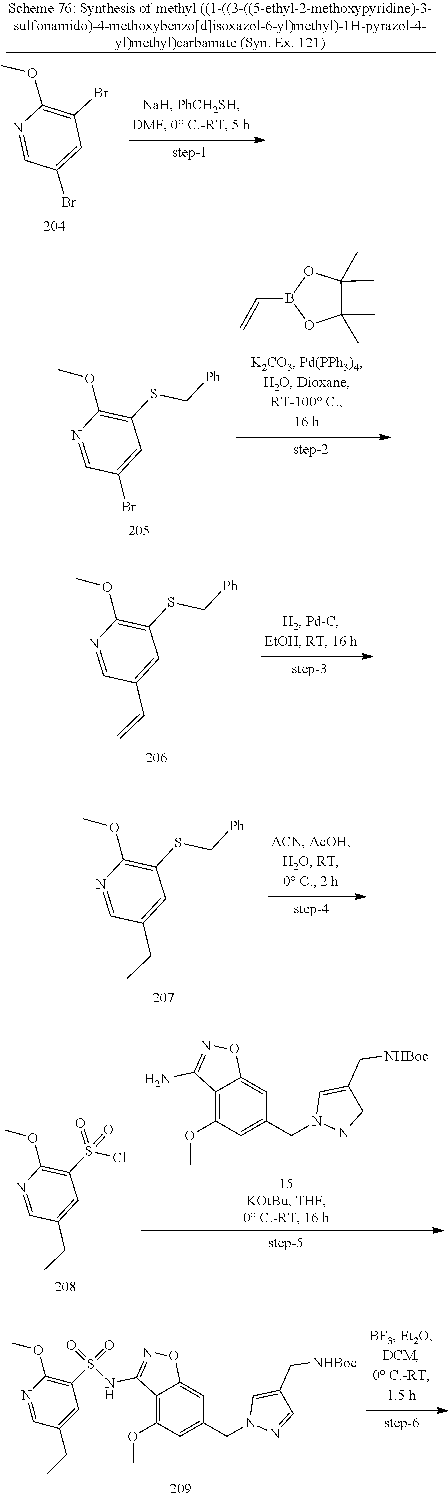

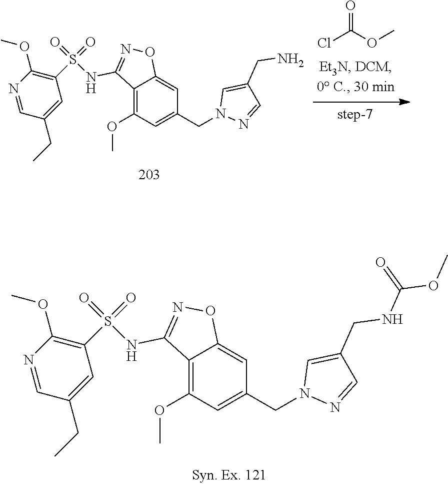

### Synthesis of 3-(benzylthio)-5-bromo-2-methoxypyridine (205)

To a stirred solution of compound 204 (13 g, 48.703 mmol) in DMF (60 mL) were added 60% NaH in Oil (2.33 g, 58.444 mmol) and PhCH₂SH (6.27 mL, 53.574 mmol) at 0° C. The reaction mixture was stirred at room temperature for 5 h. After completion (monitored by TLC), the mixture was quenched with ice-cold water and stirred at 0° C. for 10 min. The solid precipitate was filtered and dried under reduced pressure to afford title compound 205 (10 g, 64.20%) as a pale-yellow solid. LCMS calculated for C13H12BrNOS: 308.98; Found: 310 (M+2).

### Synthesis of 3-(benzylthio)-2-methoxy-5-vinylpyridine (206)

To a stirred solution of compound 205 (5 g, 16.118 mmol) in 1,4-Dioxane (40 mL) and water (10 mL) were added K₂CO₃ (4.45 g, 32.236 mmol) and 4,4,5,5-tetramethyl-2-vinyl-1,3,2-dioxaborolane (3.72 g, 24.177 mmol) at room temperature. The reaction mixture was degassed with N₂ for 10 min followed by addition of PdCl₂(PPh₃)₂ (0.56 g, mmol). The reaction mixture was stirred at 100° C. for 16 h. After completion (monitored by TLC), the mixture was filtered through a celite bed and washed with EtOAc. The filtrate was washed with water, dried over anhydrous Na₂SO₄, filtered and concentrated under reduced pressure to afford title compound 206 (3.50 g, 69.19%) as a colourless liquid. LCMS calculated for C15H15NOS: 257.09; Found; 258.10 (M+1).

### Synthesis of 3-(benzylthio)-5-ethyl-2-methoxypyridine (207)

To a stirred solution of compound 206 (3.80 g, 14.765 mmol) in EtOH (40 mL) was added 10% Pd—C(0.37 g, 3.543 mmol). The reaction mixture was stirred at room temperature for 16 h under H₂ atmosphere. After completion (monitored by TLC), the reaction mixture was filtered through a celite bed and washed with MeOH. The filtrate was concentrated under reduced pressure to afford title compound 207 (3 g, 74.41%) as a reddish semi-solid. LCMS calculated for C15H17NOS: 259.10; Found: 260.10 (M+1).

### Synthesis of 5-ethyl-2-methoxypyridine-3-sulfonyl chloride (208)

To a stirred solution of compound 207 (0.50 g, 1.927 mmol) in ACN (10 mL) were added water (1.50 mL) and AcOH (2.50 mL) followed by the addition of 1,3-dichloro-5,5-dimethylhydantoin (0.75 g, 3.855 mmol) at 0° C. The reaction mixture was stirred at 0° C. for 2 h. After completion (monitored by TLC), the mixture was quenched with ice-cold water and extracted with EtOAc. The combined organic layer was dried over anhydrous Na₂SO₄ and concentrated under reduced pressure. The crude was purified by combi-flash chromatography using 20% EtOAc/Heptane to title compound 208 (0.25 g, 36.86%) as an off-white sticky solid. LCMS calculated for C8H10ClNO3S: 235.01; Found: 236.10 (M+1).

### Synthesis of tert-Butyl ((1-((3-((5-ethyl-2-methoxypyridine)-3-sulfonamido)-4-methoxybenzo[d]isoxazol-6-yl)methyl)-1H-pyrazol-4-yl)methyl)carbamate (209)

To a stirred solution of compound 15 (0.25 g, 0.669 mmol) in THF (10 mL) was added 1M KOtBu in THF (2 mL, 2.008 mmol) at 0° C. The reaction mixture was stirred at 0° C. for 10 min followed by addition of 5-ethyl-2-methoxypyridine-3-sulfonyl chloride (0.31 g, 1.339 mmol). The reaction mixture was stirred at room temperature for 16 h. After completion (monitored by TLC), the mixture was quenched with ice-cold water, acidified with citric acid and extracted with EtOAc. The organic layer was dried over anhydrous Na₂SO₄, filtered and concentrated under reduced pressure. The crude was purified by combi-flash chromatography using 70% EtOAc/Heptane to afford compound 209 (0.20 g, 42.93%) as an off-white solid. LCMS calculated for C26H32N6O7S: 572.21; Found: 571.30 (M−1).

### Synthesis of N-(6-((4-(aminomethyl)-1H-pyrazol-1-yl)methyl)-4-methoxybenzo[d]isoxazol-3-yl)-5-ethyl-2-methoxypyridine-3-sulfonamide (210)

To a stirred solution of compound 209 (0.18 g, 0.314 mmol) in DCM (10 mL) was added BF₃·Et₂O (0.50 mL) at 0° C. The reaction mixture was stirred at room temperature for 1.5 h. After completion (monitored by TLC), the mixture was concentrated under reduced pressure. The solid residue was washed Et2O and dried under reduced pressure to afford title compound 210 (0.15 g, 88.07%) as a pale-yellow solid. LCMS calculated for C21H24N6O5S: 472.15; Found: 471.20 (M−1).

### Synthesis of methyl ((1-((3-((5-ethyl-2-methoxypyridine)-3-sulfonamido)-4-methoxybenzo[d]isoxazol-6-yl)methyl)-1H-pyrazol-4-yl)methyl)carbamate (Syn. Ex. 121)

To a stirred solution of compound 210 (0.10 g, 0.211 mmol) in DCM (10 mL) were added Et₃N (0.08 mL, 0.634 mmol) and methyl chloroformate (0.02 g, 0.253 mmol) at 0° C. The reaction mixture was stirred at 0° C. for 30 min. After completion (monitored by TLC), the mixture was diluted with water and extracted with EtOAc. The combined organic layer was dried over anhydrous Na₂SO₄, filtered and concentrated under reduced pressure. The crude was purified by prep. HPLC to afford methyl((1-((3-((5-ethyl-2-methoxypyridine)-3-sulfonamido)-4-methoxybenzo[d]isoxazol-6-yl)methyl)-1H-pyrazol-4-yl)methyl)carbamate (Syn. Ex. 121; 20 mg, 17.65%) as an off-white solid. (See analytical data in Table 1).

### Example 122

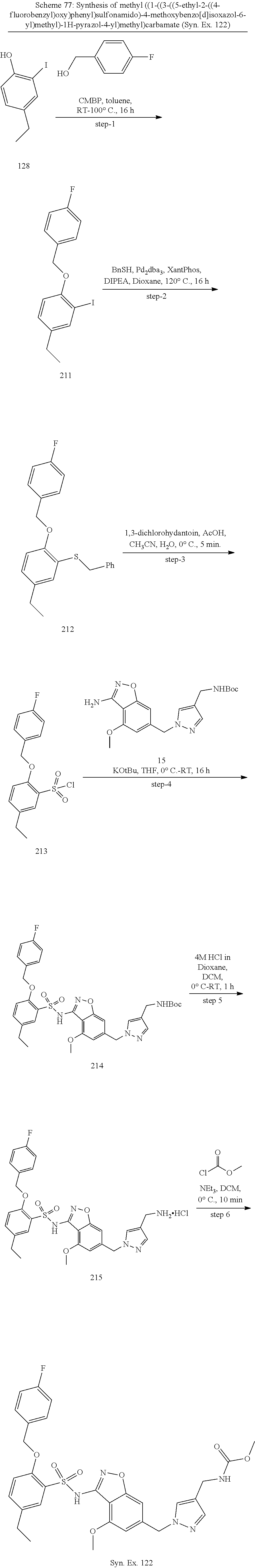

### Synthesis of 4-ethyl-1-((4-fluorobenzyl)oxy)-2-iodobenzene (211)

To a stirred solution of compound 128 (5 g, 20.156 mmol) in toluene (50 mL) were added (4-fluorophenyl)methanol (3.81 g, 30.235 mmol) and cyanomethylenetributylphosphorane (CMPB, 7.44 g, 30.235 mmol) at room temperature. The reaction mixture was stirred at 110° C. for 16 h. After completion (monitored by TLC), the mixture was quenched with ice-cold water and extracted with EtOAc. The combined organic layer was dried over anhydrous Na₂SO₄, concentrated under reduced pressure and by combi-flash column chromatography using 5% EtOAc/Heptane to afford title compound 211 (3 g, 41.78%) as a pale-yellow solid. ¹H NMR (400 MHz, DMSO-d₆) δ ppm 7.62 (br s, 1H), 7.53 (br t, J=6.26 Hz, 2H), 7.14-7.28 (m, 3H), 6.99 (br d, J=8.22 Hz, 1H), 5.13 (s, 2H), 1.13 (br t, J=7.43 Hz, 3H).

### Synthesis of benzyl(5-ethyl-2-((4-fluorobenzyl)oxy)phenyl)sulfane (212)

To a stirred solution of compound 211 (3 g, 8.422 mmol) in 1,4-Dioxane (50 mL) were added DIPEA (3.24 mL, 18.529 mmol), XantPhos (0.40 g, 0.673 mmol) and BnSH (1.04 g, 8.422 mmol). The reaction mixture was degassed with N₂ for 10 min followed by addition of Pd₂(dba)₃ (0.38 g, 0.421 mmol). The reaction mixture was stirred at 120° C. for 16 h. After completion (monitored by TLC), the mixture was quenched with ice-cold water and extracted with EtOAc. The organic layer was washed with brine, dried over anhydrous Na₂SO₄, filtered and concentrated under reduced pressure. The crude was purified by combi-flash column chromatography using 5% EtOAc/Heptane to afford title compound 212 (2.70 g, 47.29%) as a pale-yellow solid. LCMS calculated for C22H21FOS: 352.13; Found: 353.26 (M+1).

### Synthesis of 5-ethyl-2-((4-fluorobenzyl)oxy)benzenesulfonyl chloride (213)

To a stirred solution of compound 212 (1 g, 2.837 mmol) in ACN (30 mL), AcOH (1 mL) and water (1 mL) was added 1,3-dichloro-5,5-dimethylhydantoin (0.84 g, 4.255 mmol) at 0° C. The reaction mixture was stirred at 0° C. for 5 min. After completion (monitored by TLC), the mixture was quenched with ice-cold water and extracted with EtOAc. The combined organic layer was washed with saturated NaHCO₃, dried over anhydrous Na₂SO₄, filtered and concentrated under reduced pressure. The crude was purified by combi-flash column chromatography using 5% EtOAc/Heptane to afford title compound 213 (0.65 g, 21.11%) as a white solid. The crude was carried forward to the next step without purification.

### Synthesis of tert-Butyl ((1-((3-((5-ethyl-2-((4-fluorobenzyl)oxy)phenyl)sulfonamido)-4-methoxybenzo[d]isoxazol-6-yl)methyl)-1H-pyrazol-4-yl)methyl)carbamate (214)

To a stirred solution of compound 15 (0.20 g, 0.535 mmol) in THF (15 mL) were added 1M KOtBu in THF (1.60 mL, 1.606 mmol) and compound 213 (0.35 g, 1.071 mmol) at 0° C. The reaction mixture was stirred at room temperature for 16 h. After completion (monitored by TLC), the mixture was quenched with aqueous citric acid and extracted with EtOAc. The combined organic layer was washed with brine, dried over anhydrous Na₂SO₄ and concentrated under reduced pressure. The crude was purified by combi-flash chromatography using 5% EtOAc/Heptane to afford title compound 214 (0.19 g, 47.96%) as a white solid. LCMS calculated for C33H36FN5O7S: 665.23; Found: 664.30 (M−1).

### Synthesis of N-(6-((4-(aminomethyl)-1H-pyrazol-1-yl)methyl)-4-methoxybenzo[d]isoxazol-3-yl)-5-ethyl-2-((4-fluorobenzyl)oxy)benzenesulfonamide hydrochloride (215)

To a stirred solution of compound 214 (0.03 g, 0.045 mmol) in DCM (2 mL) was added 4M HCl in Dioxane (0.30 mL) at 0° C. The reaction mixture was stirred at room temperature for 1 h. After completion (monitored by TLC), the mixture was concentrated under reduced pressure. The solid residue was washed with Et₂O and dried under reduced pressure to afford title compound 215 (0.02 g, 68.72%) as an off-white solid. LCMS calculated for C28H28FN5O5S: 565.18; Found: 564.20 (M−1).

### Synthesis of methyl ((1-((3-((5-ethyl-2-((4-fluorobenzyl)oxy)phenyl)sulfonamido)-4-methoxybenzo[d]isoxazol-6-yl)methyl)-1H-pyrazol-4-yl)methyl)carbamate (Syn. Ex. 122)

To a stirred solution of N-(6-((4-(aminomethyl)-1H-pyrazol-1-yl)methyl)-4-methoxybenzo[d]isoxazol-3-yl)-5-ethyl-2-((4-fluorobenzyl)oxy)benzenesulfonamide hydrochloride (215) (0.15 g, 0.216 mmol) in DCM (5 mL) were added Et₃N (0.09 mL, 0.650 mmol) and methyl carbonochloridate (0.02 g, 0.260 mmol) at 0° C. The reaction mixture was stirred at the same temperature for 10 min. After completion (monitored by TLC), the mixture was quenched with ice-cold water and extracted with EtOAc (2×10 mL). The combined organic layer was dried over anhydrous Na₂SO₄ and concentrated under reduced pressure. The crude was purified by prep. HPLC to afford methyl ((1-((3-((5-ethyl-2-((4-fluorobenzyl)oxy)phenyl)sulfonamido)-4-methoxybenzo[d]isoxazol-6-yl)methyl)-1H-pyrazol-4-yl)methyl)carbamate (Syn. Ex. 122; 8 mg, 5.82%) as a white solid. (See analytical data in Table 1).

### Example 123

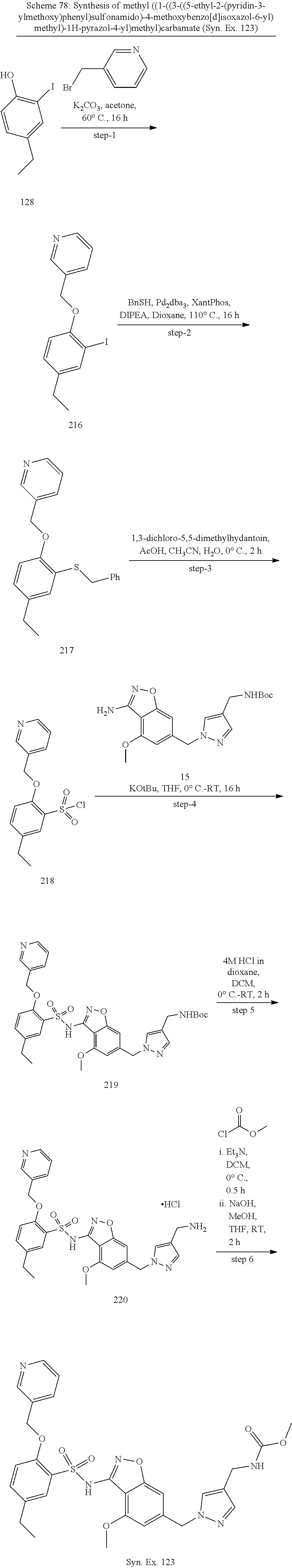

### Synthesis of 3-((4-ethyl-2-iodophenoxy)methyl)pyridine (216)

To a stirred solution of compound 128 (2 g, 8.062 mmol) in acetone (25 mL) were added K₂CO₃ (2.78 g, 20.156 mmol) and 3-(bromomethyl)pyridine (1.52 g, 8.868 mmol). The reaction mixture was stirred at 60° C. for 16 h. After completion (monitored by TLC), the mixture was quenched with water and extracted with EtOAc. The organic layer was dried over anhydrous Na₂SO₄, filtered and concentrated under reduced pressure. The crude was purified by combi-flash chromatography using 20-25% EtOAc/Heptane to afford title compound 216 (1.60 g, 57.86%) as a brown oil. LCMS calculated for C14H14INO: 339.01; Found: 340 (M+1).

### Synthesis of 3-((2-(benzylthio)-4-ethylphenoxy)methyl)pyridine (217)

To a stirred solution of compound 216 (1.60 g, 4.717 mmol) in 1,4-Dioxane (20 mL) were added DIPEA (1.80 mL, 10.377 mmol), BnSH (0.58 g, 4.717 mmol) and XantPhos (0.22 g, 0.377 mmol). The reaction mixture was degassed with Argon purging for 10 min followed by addition of Pd₂(dba)₃ (0.22 g, 0.235 mmol). The reaction mixture was stirred at 110° C. for 16 h. After completion (monitored by TLC), the mixture was quenched with ice-cold water, extracted with EtOAc. The organic layer was washed with brine, dried over anhydrous Na₂SO₄ and concentrated under reduced pressure. The crude was purified by combi-flash column chromatography using 35-40% EtOAc/Heptane to afford title compound 217 (1.50 g, 93.75%) as a pale-yellow solid. LCMS calculated for C21H21NOS: 335.13; Found: 336.10 (M+1).

### Synthesis of 5-ethyl-2-(pyridin-3-ylmethoxy)benzenesulfonyl chloride (218)

To a stirred solution of compound 217 (0.80 g, 2.384 mmol) in ACN (32 mL), AcOH (1.60 mL) and H₂O (0.80 mL) was added 1,3-dichloro-5,5-dimethylhydantoin (0.94 g, 4.769 mmol) at 0° C. The reaction mixture was stirred at 0° C. for 2 h. After completion (monitored by TLC), the mixture was quenched with ice-cold water and extracted with EtOAc. The combined organic layer was washed with saturated NaHCO₃, dried over anhydrous Na₂SO₄, filtered and concentrated under reduced pressure to afford title compound 218 (0.83 g, 31.20%) as a brown gummy solid. The crude was carried in the next step without purification.

### Synthesis of tert-Butyl ((1-((3-((5-ethyl-2-(pyridin-3-ylmethoxy)phenyl)sulfonamido)-4-methoxybenzo[d]isoxazol-6-yl)methyl)-1H-pyrazol-4-yl)methyl)carbamate (219)

To a stirred solution of compound 15 (0.30 g, 0.80 mmol) in THF (4 mL) were added compound 218 (0.37 g, 1.20 mmol) and 1M KOtBu in THF (2.41 mL, 2.41 mmol) at 0° C. The reaction mixture was stirred at room temperature for 16 h. After completion (monitored by TLC), the mixture was quenched with saturated citric acid and extracted with EtOAc. The combined organic layer was washed with brine, dried over anhydrous Na₂SO₄, filtered and concentrated under reduced pressure. The crude was purified by combi-flash chromatography using 2-5% MeOH/DCM to afford title compound 219 (0.11 g, 15.32%) as a brown solid. LCMS calculated for C32H36N6O7S: 648.24; Found: 649.30 (M+1).

### Synthesis of N-(6-((4-(aminomethyl)-1H-pyrazol-1-yl)methyl)-4-methoxybenzo[d]isoxazol-3-yl)-5-ethyl-2-(pyridin-3-ylmethoxy)benzenesulfonamide hydrochloride (220)

To a stirred solution of compound 219 (0.08 g, 0.123 mmol) in DCM (4 mL) was added 4M HCl in Dioxane (0.15 mL, 0.616 mmol) at 0° C. The reaction mixture was stirred at room temperature for 2 h. After completion (monitored by TLC), the mixture was concentrated under reduced pressure and triturated with Et₂O to afford title compound 220 (0.075 g, 74.47%) as a brown solid. LCMS calculated for C27H28N6O5S: 548.18; Found: 547.20 (M−1).

### Synthesis of methyl ((1-((3-((5-ethyl-2-(pyridin-3-ylmethoxy)phenyl)sulfonamido)-4-methoxybenzo[d]isoxazol-6-yl)methyl)-1H-pyrazol-4-yl)methyl)carbamate (Syn. Ex. 123)

To a stirred solution of compound 220 (0.075 g, 0.128 mmol) in DCM (4 mL) were added Et₃N (0.05 mL, 0.384 mmol) and methyl carbonochloridate (0.01 g, 0.153 mmol) at 0° C. while maintaining the pH at 7-8. The reaction mixture was stirred at 0° C. for 30 min. After completion (monitored by TLC), the mixture was concentrated under reduced pressure to afford crude. The crude was dissolved in solution of MeOH:THF (6 mL; 2:1) followed by addition of NaOH (10 mg). The mixture was stirred at room temperature for 1 h. After completion (monitored by TLC), the mixture was concentrated under reduced pressure. The crude was purified by prep. HPLC to afford methyl ((1-((3-((5-ethyl-2-(pyridin-3-ylmethoxy)phenyl)sulfonamido)-4-methoxybenzo[d]isoxazol-6-yl)methyl)-1H-pyrazol-4-yl)methyl)carbamate (Syn. Ex. 123; 19.5 mg, 25.16%) as an off-white solid. (See analytical data in Table 1).

### Example 124

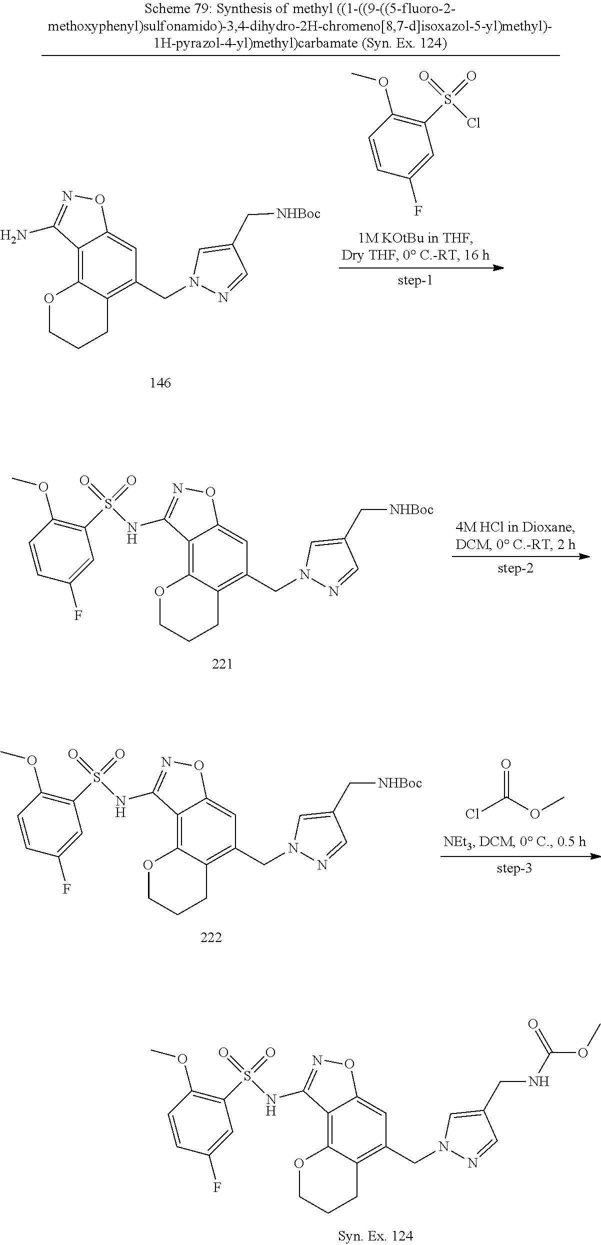

### Synthesis of tert-Butyl ((1-((9-((5-fluoro-2-methoxyphenyl)sulfonamido)-3,4-dihydro-2H-chromeno[8,7-d]isoxazol-5-yl)methyl)-1H-pyrazol-4-yl)methyl)carbamate (221)

To a stirred solution of compound 146 (1 g, 2.503 mmol) in dry THF (10 mL) were added 1M KOtBu in THF (7.51 mL, 7.510 mmol) and 5-fluoro-2-methoxybenzenesulfonyl chloride (1.12 g, 5.006 mmol) at 0° C. The reaction mixture was stirred at room temperature for 16 h. After completion (monitored by TLC), the mixture was quenched with ice-cold water and extracted with EtOAc. The aqueous layer was acidified with 1N HCl to adjust the pH ˜6 and extracted with EtOAc. The organic layer was washed with brine, dried over anhydrous Na₂SO₄, filtered and concentrated under reduced pressure to afford title compound 221 (1 g, 37.38%) as a brown solid. LCMS calculated for C27H30FN5O7S: 587.18; Found: 586.20 (M−1).

### Synthesis of N-(5-((4-(aminomethyl)-1H-pyrazol-1-yl)methyl)-3,4-dihydro-2H-chromeno[8,7-d]isoxazol-9-yl)-5-fluoro-2-methoxybenzenesulfonamide hydrochloride (222)

To a stirred solution of compound 221 (1 g, 1.701 mmol) in DCM (10 mL) was added 4M HCl in Dioxane (2.12 mL, 8.508 mmol) at 0° C. The reaction mixture was stirred at room temperature for 2 h. After completion (monitored by TLC), the mixture was concentrated under reduced pressure. The solid residue was washed with DCM, co-distilled with Et₂O and concentrated under reduced pressure to afford title compound 222 (0.50 g, 46.54%) as an off-white solid. LCMS calculated for C22H22FN5O5S: 487.13; Found: 486.10 (M−1).

### Synthesis of methyl ((1-((9-((5-fluoro-2-methoxyphenyl)sulfonamido)-3,4-dihydro-2H-chromeno[8,7-d]isoxazol-5-yl)methyl)-1H-pyrazol-4-yl)methyl)carbamate (Syn. Ex. 124)

To a stirred solution of compound 222 (0.20 g, 0.381 mmol) in DCM (10 mL) were added NEt₃ (0.10 mL, 0.763 mmol) and methyl chloroformate (0.05 g, 0.572 mmol) at 0° C. The reaction mixture was stirred at 0° C. for 30 min. After completion (monitored by TLC), the mixture was diluted with water and extracted with DCM. The combined organic layer was dried over anhydrous Na₂SO₄, filtered and concentrated under reduced pressure. The crude was purified by prep. HPLC to afford methyl ((1-((9-((5-fluoro-2-methoxyphenyl)sulfonamido)-3,4-dihydro-2H-chromeno[8,7-d]isoxazol-5-yl)methyl)-1H-pyrazol-4-yl)methyl)carbamate (Syn. Ex. 124; 7 mg, 3.26%) as an off-white solid. (See analytical data in Table 1).

### Example 125

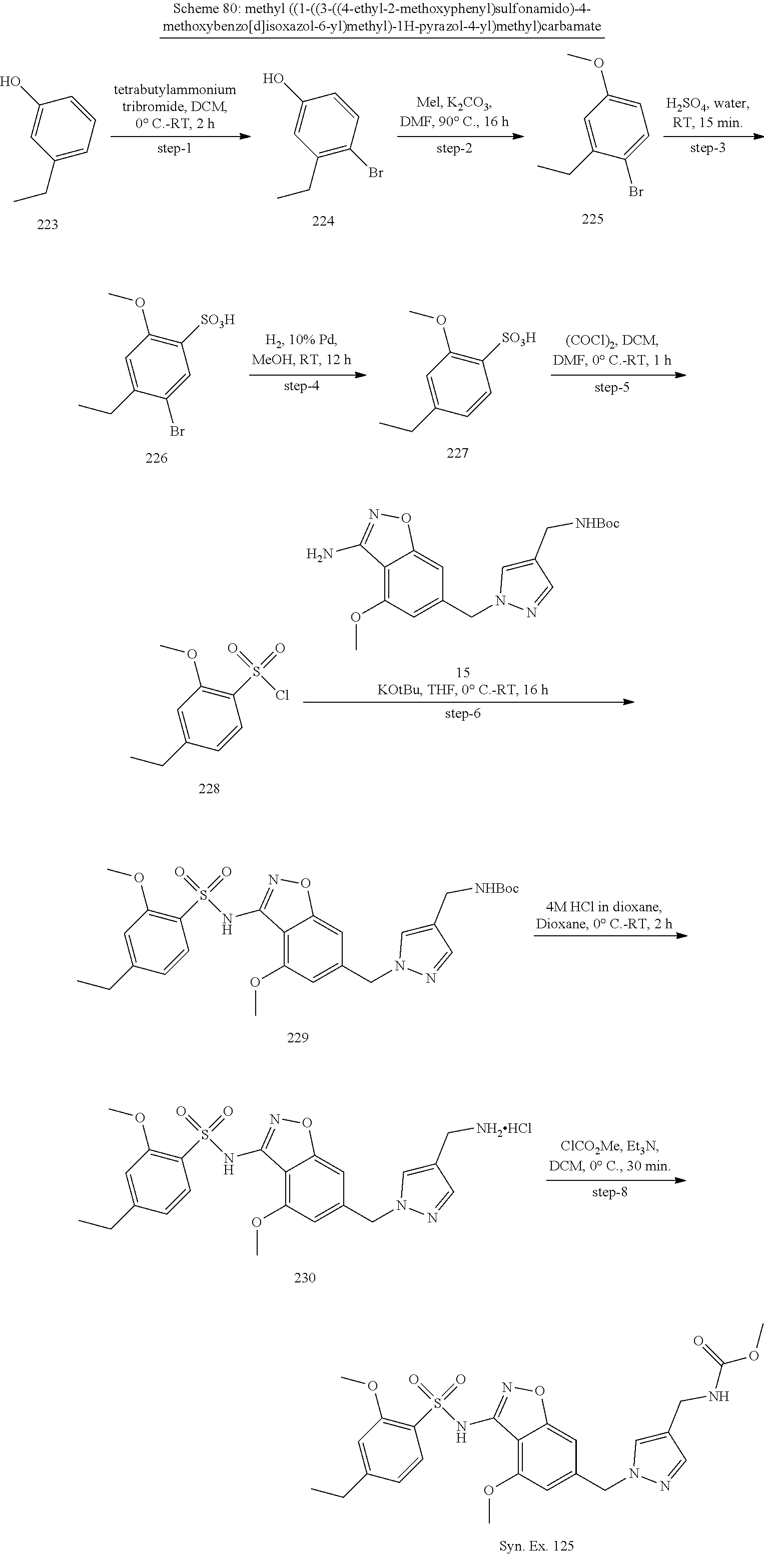

### Synthesis of 4-bromo-3-ethylphenol (224)

To a stirred solution of 3-ethylphenol 223 (10 g, 81.853 mmol) in DCM (100 mL) was added tetrabutylammonium tribromide (39.46 g, 81.853 mmol) at 0° C. The reaction mixture was stirred at room temperature for 2 h. After completion (monitored by TLC), the mixture was concentrated under reduced pressure. The residue was washed with DCM, co-distilled with Et₂O and dried under reduced pressure to afford title compound 224 (10 g, 43.14%) as a liquid. LCMS calculated for C8H9BrO: 199.98; Found: 201.10 (M+1).

### Synthesis of 1-bromo-2-ethyl-4-methoxybenzene (225)

To a stirred solution of compound 224 (10 g, 49.736 mmol) in DMF (100 mL) were added K₂CO₃ (13.74 g, 99.472 mmol) and Mel (6.19 mL, 99.472 mmol). The reaction mixture was stirred at 90° C. for 16 h. After completion (monitored by TLC), the mixture was quenched with water and extracted with EtOAc. The organic layer was dried over anhydrous Na₂SO₄ and concentrated under reduced pressure to afford title compound 225 (10 g, 93.47%) as a liquid. ¹H NMR (400 MHz, DMSO-d₆) δ ppm 7.44 (br d, J=8.77 Hz, 1H), 6.92 (br d, J=1.75 Hz, 1H), 6.74 (br dd, J=8.55, 2.41 Hz, 1H), 3.74 (s, 3H), 2.65 (q, J=7.45 Hz, 2 H), 1.15 (br t, J=7.23 Hz, 3H).

### Synthesis of 5-bromo-4-ethyl-2-methoxybenzenesulfonic acid (226)

Compound 225 (2 g, 9.298 mmol) in was dissolved in H₂SO₄ (50 mL). The reaction mixture was stirred at room temperature for 15 min. After completion (monitored by TLC), the mixture was quenched with ice-cold water (50 mL), filtered and washed with heptane. The solid precipitate was dried under reduced pressure to afford title compound 226 (2.50 g, 91.09%) as a white solid. LCMS calculated for C9H11BrO4S: 293.96; Found: 295 (M+1).

### Synthesis of 4-ethyl-2-methoxybenzenesulfonic acid (227)

To a stirred solution of 5-bromo-4-ethyl-2-methoxybenzenesulfonic acid 226 (5 g, 16.940 mmol) in MeOH (60 mL) was added 10% Pd (0.54 g, 0.508 mmol). The reaction mixture was stirred at room temperature for 12 h under H₂ atmosphere (50 psi). After completion (monitored by TLC), the reaction mixture was filtered through a celite bed and the filtrate was concentrated under reduced pressure to afford 4-ethyl-2-methoxybenzenesulfonic acid 227 (2 g, 51.31%) as a brown solid. LCMS calculated for C9H12O4S: 216.05; Found: 215.10 (M−1).

### Synthesis of 4-ethyl-2-methoxybenzenesulfonyl chloride (228)

To a solution of 4-ethyl-2-methoxybenzenesulfonic acid 227 (0.60 g, 2.774 mmol) in DCM (10 mL) and DMF (0.02 mL, 0.277 mmol) was added (COCl)₂ (2.42 mL, 27.745 mmol) at 0° C. The reaction mixture was stirred at room temperature for 1 h. After completion (monitored by TLC), the mixture was quenched with ice-cold water and extracted with EtOAc. The combined organic layer was washed with brine, dried over anhydrous Na₂SO₄, filtered and concentrated under reduced pressure to afford title compound 228 (0.45 g, 45.61%) as a brown liquid. ¹H NMR (401 MHz, DMSO-d₆) δ ppm 7.56 (d, J=7.82 Hz, 1 H), 6.81 (s, 1H), 6.70 (d, J=7.58 Hz, 1H), 3.74 (s, 3H), 2.58 (q, J=7.58 Hz, 2H), 1.17 (t, J=7.58 Hz, 3H).

### Synthesis of tert-Butyl ((1-((3-((4-ethyl-2-methoxyphenyl)sulfonamido)-4-methoxybenzo[d]isoxazol-6-yl)methyl)-1H-pyrazol-4-yl)methyl)carbamate (229)

To a stirred solution of compound 15 (0.30 g, 0.803 mmol) in THF (10 mL) was added 1M KOtBu in THF (2.41 mL, 2.410 mmol) at 0° C. The reaction mixture was stirred at same temperature for 10 min. followed by compound 228 (0.37 g, 1.606 mmol) was added. The reaction mixture was stirred at room temperature for 16 h. After completion (monitored by TLC), the mixture was quenched with ice-cold water and extracted with EtOAc. The aqueous layer was acidified with 1N HCl and extracted with EtOAc. The combined organic layer was washed with brine, dried over anhydrous Na₂SO₄, filtered and concentrated under reduced pressure to afford title compound 229 (0.15 g, 23.51%) as a brown solid. LCMS calculated for C27H33N5O7S: 571.21; Found: 570.20 (M−1).

### Synthesis of N-(6-((4-(aminomethyl)-1H-pyrazol-1-yl)methyl)-4-methoxybenzo[d]isoxazol-3-yl)-4-ethyl-2-methoxybenzenesulfonamide hydrochloride (230)

To a stirred solution of compound 229 (0.15 g, 0.262 mmol) in 1,4-Dioxane (5 mL) was added 4M HCl in Dioxane (0.32 mL, 1.311 mmol) at 0° C. The reaction mixture was stirred at room temperature for 2 h. After completion (monitored by TLC), the mixture was concentrated under reduced pressure. The crude was washed with DCM, co-distilled with Et₂O and concentrated under reduced pressure to afford title compound 230 (0.12 g, 69.32%) as an off-white solid. LCMS calculated for C22H25N5O5S: 471.16; Found: 470.20 (M−1).

### Synthesis of methyl ((1-((3-((4-ethyl-2-methoxyphenyl)sulfonamido)-4-methoxybenzo[d]isoxazol-6-yl)methyl)-1H-pyrazol-4-yl)methyl)carbamate (Syn. Ex. 125)

To a stirred solution of compound 230 (0.12 g, 0.236 mmol) in DCM (10 mL) were added Et₃N (0.06 mL, 0.472 mmol) and methyl chloroformate (0.02 g, 0.283 mmol) at 0° C. The reaction mixture was stirred at 0° C. for 30 min. After completion (monitored by TLC), the mixture was quenched with water and extracted with DCM. The combined organic layer was dried over anhydrous Na₂SO₄, filtered and concentrated under reduced pressure. The crude was purified by prep. HPLC to afford methyl ((1-((3-((4-ethyl-2-methoxyphenyl)sulfonamido)-4-methoxybenzo[d]isoxazol-6-yl)methyl)-1H-pyrazol-4-yl)methyl)carbamate (Syn. Ex. 125; 8.00 mg, 6.33%) as an off-white solid. See analytical data in Table 1.

### Example 126

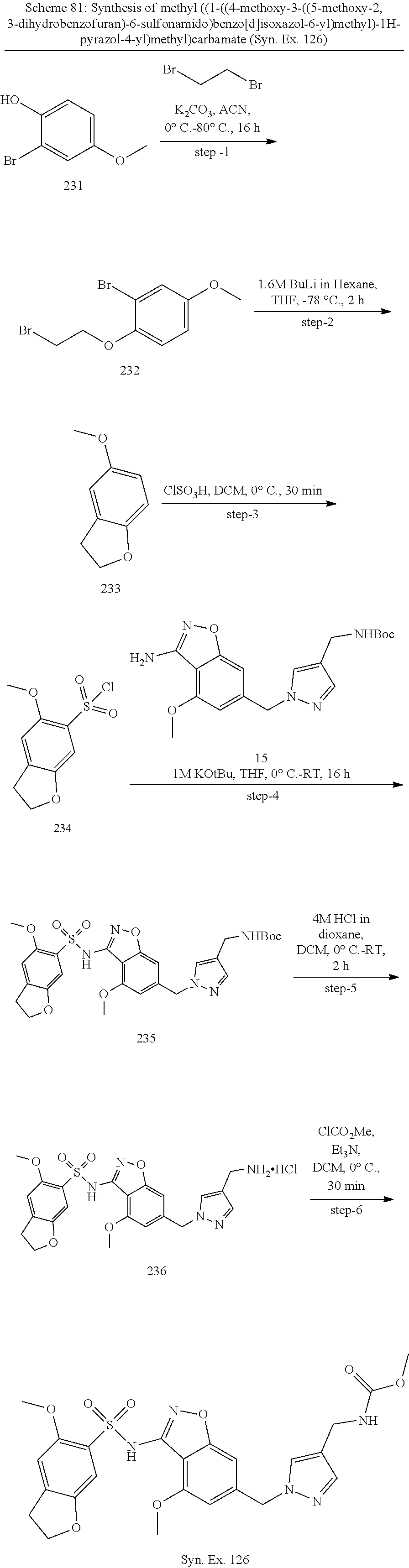

### Synthesis of 2-bromo-1-(2-bromoethoxy)-4-methoxybenzene (232)

To a stirred solution of compound 231 (10 g, 49.251 mmol) in ACN (100 mL) were added K₂CO₃ (14.64 g, 147.754 mmol) at 0° C. and 1,2-dibromoethane (27.71 g, 147.754 mmol). The reaction mixture was stirred at 80° C. for 16 h. After completion (monitored by TLC), the mixture was quenched with ice-cold water and extracted with EtOAc. The organic layer was dried over anhydrous Na₂SO₄, filtered and concentrated under reduced pressure. The crude was purified by combi-flash column chromatography using 10-20% EtOAc/Heptane to afford title compound 232 (6 g, 38.51%) as an off-white solid; ¹H NMR (400 MHz, DMSO-d₆) δ ppm 7.18 (d, J=3.07 Hz, 1H), 7.09 (d, J=9.21 Hz, 1H), 6.92 (dd, J=8.99, 2.85 Hz, 1H), 4.31 (t, J=5.48 Hz, 2H), 3.78 (t, J=5.48 Hz, 2H), 3.72 (s, 3H).

### Synthesis of 5-methoxy-2,3-dihydrobenzofuran (233)

To a stirred solution of compound 232 (5 g, 16.130 mmol) in THF (50 mL) was added BuLi (1.6 mol/L) in Hexane (12.09 mL, 19.356 mmol) at −78° C. The reaction mixture was stirred at −78° C. for 2 h. After completion (monitored by TLC), the mixture was quenched with ice-cold water and extracted with EtOAc. The combined organic layer was dried over anhydrous Na₂SO₄, filtered and concentrated under reduced pressure to afford title compound 233 (2.30 g, 86.40%) as a colourless liquid; LCMS calculated for C9H1002: 150.07; Found: 151.20 (M+1).

### Synthesis of 5-methoxy-2,3-dihydrobenzofuran-7-sulfonyl chloride (234)

To a stirred solution of compound 233 (0.30 g, 1.997 mmol) in DCM (3 mL) was added ClSO₃H (0.41 mL, 5.992 mmol) at 0° C. The reaction mixture was stirred at 0° C. for 30 min. After completion (monitored by TLC), the mixture was quenched with ice-cold water and extracted with EtOAc. The combined organic layer was dried over anhydrous Na₂SO₄, filtered and concentrated under reduced pressure. The crude was purified by combi-flash chromatography using 25-35% EtOAc/Heptane to afford title compound 234 (0.12 g, 23.43%) as an off-white semi-solid; LCMS calculated for C9H9ClO4S: 247.99; Found: 248.74 (M+1).

### Synthesis of tert-Butyl ((1-((4-methoxy-3-((5-methoxy-2,3-dihydrobenzofuran)-6-sulfonamido)benzo[d]isoxazol-6-yl)methyl)-1H-pyrazol-4-yl)methyl)carbamate (235)

To a stirred solution of compound 15 (0.30 g, 0.803 mmol) in THF (5 mL) were added 1M KOtBu in THF (2.41 mL, 2.410 mmol) at 0° C. The reaction mixture was stirred at 0° C. for 5 min followed by addition of compound 234 (0.29 g, 1.205 mmol). The reaction mixture was stirred at room temperature for 16 h. After completion (monitored by TLC), the mixture was quenched with saturated citric acid and extracted with EtOAc. The combined organic layer was dried over anhydrous Na₂SO₄, filtered and concentrated under reduced pressure. The crude was purified by combi-flash chromatography using 80-100% EtOAc/Heptane to afford title compound 235 (0.18 g, 24.10%) as a pale-brown solid; LCMS calculated for C27H31N5O8S: 585.19; Found: 584.40 (M−1).

### Synthesis of N-(6-((4-(aminomethyl)-1H-pyrazol-1-yl)methyl)-4-methoxybenzo[d]isoxazol-3-yl)-5-methoxy-2,3-dihydrobenzofuran-6-sulfonamide hydrochloride (236)

To a stirred solution of compound 235 (0.15 g, 0.256 mmol) in DCM (2 mL) was added 4M HCl in Dioxane (0.64 mL, 2.561 mmol) at 0° C. The reaction mixture was stirred at room temperature for 2 h. After completion (monitored by TLC), the mixture was concentrated under reduced pressure. The crude was triturated with Et₂O and concentrated under reduced pressure to afford title compound 236 (0.16 g, 75.40%) as an off-white solid; LCMS calculated for C22H23N5O6S: 485.14; Found: 486.22 (M+2).

### Synthesis of methyl ((1-((4-methoxy-3-((5-methoxy-2,3-dihydrobenzofuran)-6-sulfonamido)benzo[d]isoxazol-6-yl)methyl)-1H-pyrazol-4-yl)methyl)carbamate (Syn. Ex. 126)

To a stirred solution of compound 236 (0.15 g, 0.287 mmol) in DCM (5 mL) was added Et₃N (0.08 mL, 0.574 mmol) and ClCO₂Me (0.02 g, 0.287 mmol) at 0° C. The reaction mixture was stirred at 0° C. for 30 min. After completion (monitored by TLC), the mixture was quenched with ice-cold water and extracted with DCM. The combined organic layer was dried over anhydrous Na₂SO₄, filtered and concentrated under reduced pressure. The crude was purified by prep. HPLC to afford title compound methyl ((1-((4-methoxy-3-((5-methoxy-2,3-dihydrobenzofuran)-6-sulfonamido)benzo[d]isoxazol-6-yl)methyl)-1H-pyrazol-4-yl)methyl)carbamate (Syn. Ex. 126; 0.009 g, 5.70%) as an off-white solid. See analytical data in Table 1.

### Example 127

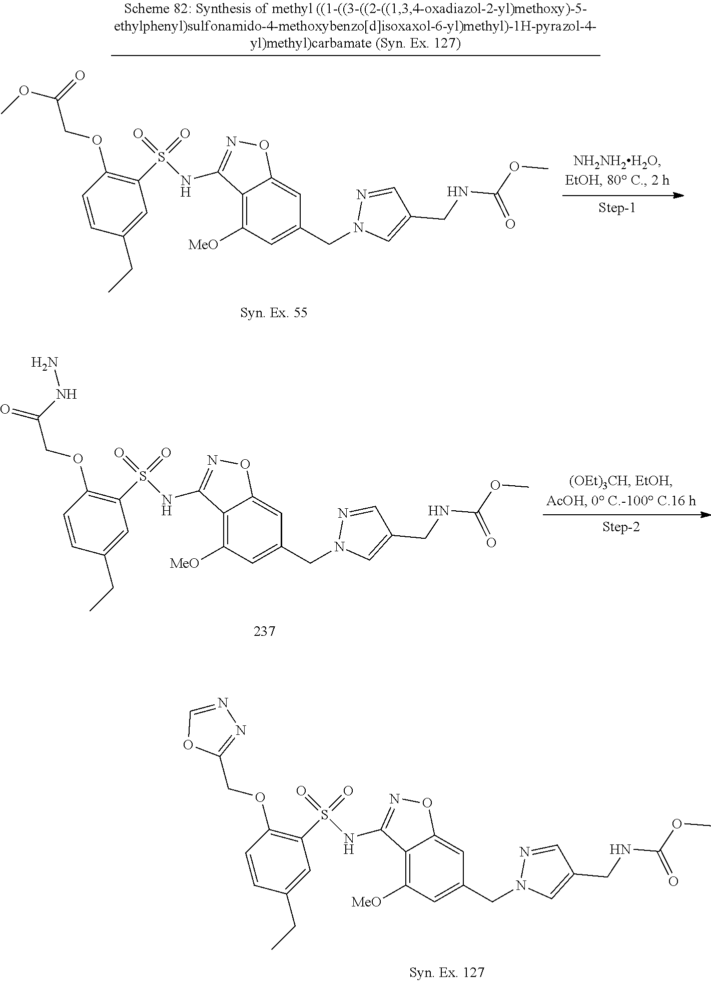

### Synthesis of methyl ((1-((3-((5-ethyl-2-(2-hydrazinyl-2-oxoethoxy)phenyl)sulfonamido)-4-methoxybenzo[d]isoxazol-6-yl)methyl)-1H-pyrazol-4-yl)methyl)carbamate (237)

To a solution of Syn. Ex. 55 (0.12 g, 0.204 mmol) in EtOH (5 mL) was added NH₂NH₂·H₂O (0.01 g, 0.245 mmol). The reaction mixture was stirred at 80° C. for 2 h. After completion (monitored by TLC), the mixture was concentrated under reduced pressure and washed with Et₂O to afford title compound 237 (0.10 g, 69.20%) as a sticky solid. LCMS calculated for C25H29N708S: 587.18; Found: 588.51 (M+1).

### Synthesis of methyl ((1-((3-((2-((1,3,4-oxadiazol-2-yl)methoxy)-5-ethylphenyl)sulfonamido)-4-methoxybenzo[d]isoxazol-6-yl)methyl)-1H-pyrazol-4-yl)methyl)carbamate (Syn. Ex. 127)

To a stirred solution of compound 237 (0.20 g, 0.183 mmol) in mixture of EtOH (2 mL) were added AcOH (0.99 mL, 17.293 mmol) and (OEt)₃CH (2 mL, 11.904 mmol). The reaction mixture was stirred at 110° C. for 16 h. After completion (monitored by TLC), the mixture was concentrated under reduced pressure. The crude was purified by prep. HPLC to afford title compound methyl ((1-((3-((2-((1,3,4-oxadiazol-2-yl)methoxy)-5-ethylphenyl)sulfonamido)-4-methoxybenzo[d]isoxazol-6-yl)methyl)-1H-pyrazol-4-yl)methyl)carbamate (Syn. Ex. 127; 5 mg, 4.41%) as an off-white solid. See analytical data in Table 1.

### Example 128

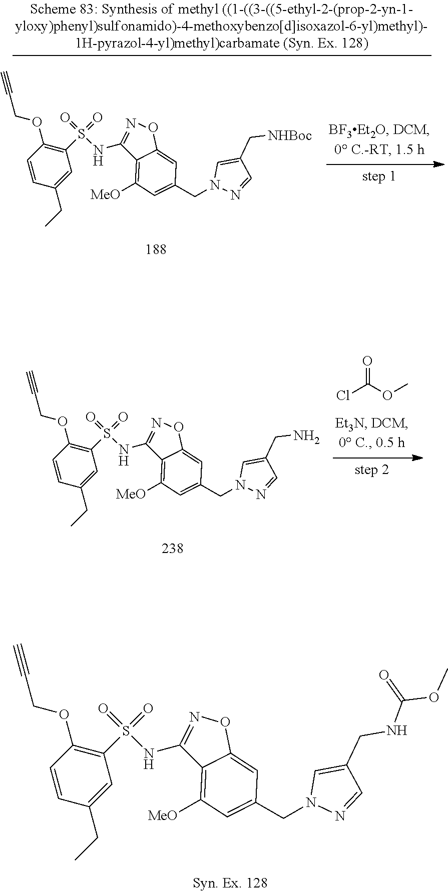

### Synthesis of N-(6-((4-(aminomethyl)-1H-pyrazol-1-yl)methyl)-4-methoxybenzo[d]isoxazol-3-yl)-5-ethyl-2-(prop-2-yn-1-yloxy)benzenesulfonamide (238)

To a stirred solution of compound 188 (50 mg, 0.083 mmol) in DCM (10 mL) was added BF₃·Et₂O (0.02 g, 0.083 mmol) at 0° C. The reaction mixture was stirred at room temperature for 1.5 h. After completion (monitored by TLC), the mixture was concentrated under reduced pressure. The residue was washed Et₂O and dried under reduced pressure to afford title compound 238 (30 mg, 32.25%) as a pale-yellow solid. LCMS calculated for C24H25N5O5S: 495.16; Found: 496.20 (M+1).

### Synthesis of methyl ((1-((3-((5-ethyl-2-(prop-2-yn-1-yloxy)phenyl)sulfonamido)-4-methoxybenzo[d]isoxazol-6-yl)methyl)-1H-pyrazol-4-yl)methyl)carbamate (Syn. Ex. 128)

To a stirred solution of compound 238 (0.15 g, 0.302 mmol) in DCM (10 mL) were added Et₃N (0.12 mL, 0.908 mmol) and methyl chloroformate (0.02 mL, 0.363 mmol) at 0° C. The reaction mixture was stirred at 0° C. for 30 min. After completion (monitored by TLC), the mixture was quenched with water and extracted with EtOAc. The combined organic layer was dried over anhydrous Na₂SO₄, filtered, and concentrated under reduced pressure. The crude was purified by prep. HPLC to afford methyl ((1-((3-((5-ethyl-2-(prop-2-yn-1-yloxy)phenyl)sulfonamido)-4-methoxybenzo[d]isoxazol-6-yl)methyl)-1H-pyrazol-4-yl)methyl)carbamate (Syn. Ex. 128; 5 mg, 2.98%) as an off-white solid. (See analytical data in Table 1).

### Example 129

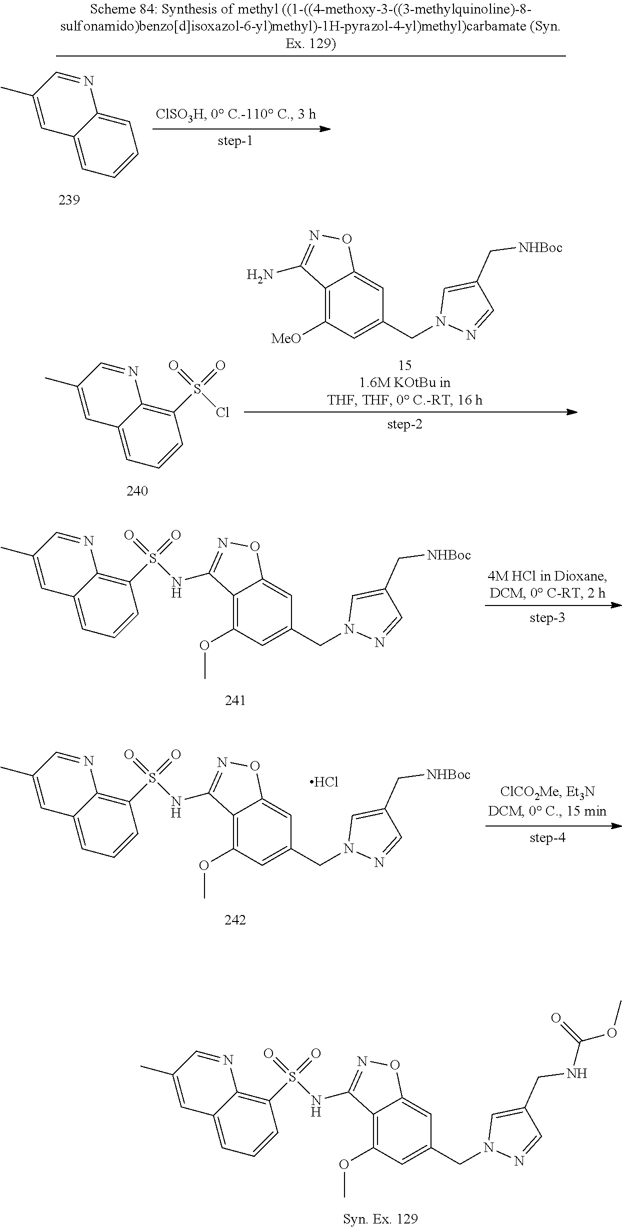

### Synthesis of 3-methylquinoline-8-sulfonyl chloride (240)

A solution of compound 239 (2 g, 13.967 mmol) in ClSO₃H (20 mL) was stirred at 110° C. for 3 h. After completion (monitored by TLC), the mixture was quenched with ice-cold water and extracted with EtOAc. The combined organic layers were washed with brine, dried over anhydrous Na₂SO₄, filtered and concentrated under reduced pressure to afford title compound 240 (2.20 g, 59.44%) as a brown solid. LCMS calculated for C10H8ClNO2S: 241.00; Found: 242.10 (M+1)

### Synthesis of tert-Butyl ((1-((4-methoxy-3-((3-methylquinoline)-8-sulfonamido)benzo[d]isoxazol-6-yl)methyl)-1H-pyrazol-4-yl)methyl)carbamate (241)

To a stirred solution of compound 15 (0.30 g, 0.803 mmol) in THF (20 mL) was added 1.6M KOtBu in THF (1.50 mL, 2.410 mmol) at 0° C. The reaction mixture was stirred for 10 min. followed by addition of compound 240 (0.33 g, 1.365 mmol) in THF. The reaction mixture was stirred at room temperature for 16 h. After completion (monitored by TLC), the mixture was quenched with water and extracted with EtOAc. The combined organic layers were dried over anhydrous Na₂SO₄, filtered and concentrated under reduced pressure. The crude was purified by combi-flash chromatography using 70-72% EtOAc/Heptane to afford title compound 241 (0.11 g, 18.45%) as an off-white solid. TLC: 80% EtOAc/Heptane (R_{f}: 0.4). LCMS calculated for C28H30N6O6S: 578.19; Found: 577.20 (M−1).

### Synthesis of N-(6-((4-(aminomethyl)-1H-pyrazol-1-yl)methyl)-4-methoxybenzo[d]isoxazol-3-yl)-3-methylquinoline-8-sulfonamide hydrochloride (242)

To a solution of compound 241 (0.11 g, 0.190 mmol) in DCM (2 mL) was added 4M HCl in dioxane (1.09 mL, 4.398 mmol) at 0° C. The reaction mixture was stirred at room temperature for 2 h. After completion (monitored by TLC), the mixture was concentrated under reduced pressure, washed with Et₂O and dried under reduced pressure to afford title compound 242 (0.09 g, 69.87%) as an off-white solid. LCMS calculated for C23H22N6O4S: 478.14; Found: 479.20 (M+1).

### Synthesis of methyl ((1-((4-methoxy-3-((3-methylquinoline)-8-sulfonamido)benzo[d]isoxazol-6-yl)methyl)-1H-pyrazol-4-yl)methyl)carbamate (Syn. Ex. 129)

To a stirred solution of compound 242 (90 mg, 0.174 mmol) in DCM (2 mL) were added triethylamine (0.07 mL, 0.524 mmol) and methyl chloroformate (0.01 g, 0.209 mmol) in DCM at 0° C. The reaction mixture was stirred at 0° C. for 15 min. After completion (monitored by TLC), the mixture was quenched with water and extracted with DCM. The combined organic layers were dried over anhydrous Na₂SO₄, filtered and concentrated under reduced pressure. The crude was purified by prep. HPLC to afford methyl ((1-((4-methoxy-3-((3-methylquinoline)-8-sulfonamido)benzo[d]isoxazol-6-yl)methyl)-1H-pyrazol-4-yl)methyl)carbamate (Syn. Ex. 129) (8 mg, 8.44%) as a white solid. (See analytical data in Table 1).

### Example 130

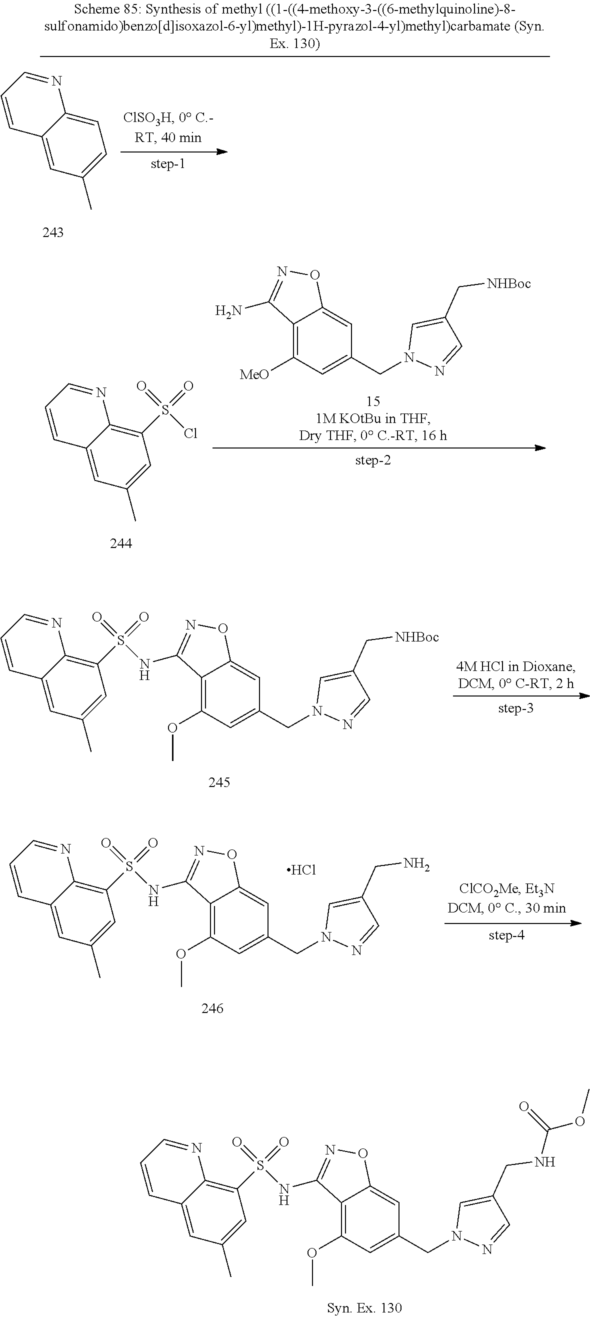

### Synthesis of 6-methylquinoline-8-sulfonyl chloride (244)

A solution of compound 243 (2 g, 13.967 mmol) in ClSO₃H (2.80 mL) was stirred at room temperature for 40 min. After completion (monitored by TLC), the mixture was quenched with ice-cold water and extracted with EtOAc. The combined organic layers were washed with brine, dried over anhydrous Na₂SO₄, filtered and concentrated under reduced pressure to afford compound 244 (1.50 g, 36.88%) as an off-white solid. LCMS calculated for C10H8ClNO2S: 241.00; Found: 242.10 (M+1).

### Synthesis of tert-Butyl ((1-((4-methoxy-3-((6-methylquinoline)-8-sulfonamido)benzo[d]isoxazol-6-yl)methyl)-1H-pyrazol-4-yl)methyl)carbamate (245)

To a stirred solution of compound 15 (0.50 g, 1.339 mmol) in THF (20 mL) was added 1M KOtBu in THF (4.01 mL, 4.017 mmol) at 0° C. The reaction mixture was stirred for 10 min. followed by addition of compound 244 (0.48 g, 2.008 mmol). The reaction mixture was stirred at room temperature for 16 h. After completion (monitored by TLC), the mixture was quenched with ice-cold water and extracted with EtOAc. The aqueous layer was acidified with 1N HCl to˜pH 6 and extracted with EtOAc. The combined organic layers were washed with brine, dried over anhydrous Na₂SO₄, filtered and concentrated under reduced pressure to afford title compound 245 (0.70 g, 52.40%) as a brown solid. LCMS calculated for C28H30N6O6S: 578.19; Found: 579.40 (M+1).

### Synthesis of N-(6-((4-(aminomethyl)-1H-pyrazol-1-yl)methyl)-4-methoxybenzo[d]isoxazol-3-yl)-6-methylquinoline-8-sulfonamide hydrochloride (246)

To a solution of compound 245 (0.70 g, 1.209 mmol) in 1,4-dioxane (4 mL) and DCM (4 mL) was added 4M HCl in Dioxane (1.512 mL, 6.048 mmol) at 0° C. The reaction mixture was stirred at room temperature for 2 h. After completion (monitored by TLC), the mixture was concentrated under reduced pressure, washed with DCM, co-distilled with Et₂O and concentrated under reduced pressure to afford title compound 246 (0.20 g, 22.15%) as an off-white solid. LCMS calculated for C23H22N6O4S: 478.14; Found: 479.20 (M+1).

### Synthesis of methyl ((1-((4-methoxy-3-((6-methylquinoline)-8-sulfonamido)benzo[d]isoxazol-6-yl)methyl)-1H-pyrazol-4-yl)methyl)carbamate (Syn. Ex. 130)

To a stirred solution of compound 246 (0.30 g, 0.582 mmol) in DCM (10 mL) were added NEt₃ (0.16 mL, 1.165 mmol) and methyl chloroformate (0.05 mL, 0.699 mmol) in DCM at 0° C. The reaction mixture was stirred at 0° C. for 30 min. After completion (monitored by TLC), the mixture was quenched with water and extracted with DCM. The combined organic layers were dried over anhydrous Na₂SO₄, filtered and concentrated under reduced pressure. The crude was purified by prep. HPLC to afford methyl ((1-((4-methoxy-3-((6-methylquinoline)-8-sulfonamido)benzo[d]isoxazol-6-yl)methyl)-1H-pyrazol-4-yl)methyl)carbamate (Syn. Ex. 130; 20 mg, 6.33%) as a white solid. (See analytical data in Table 1).

### Example 131

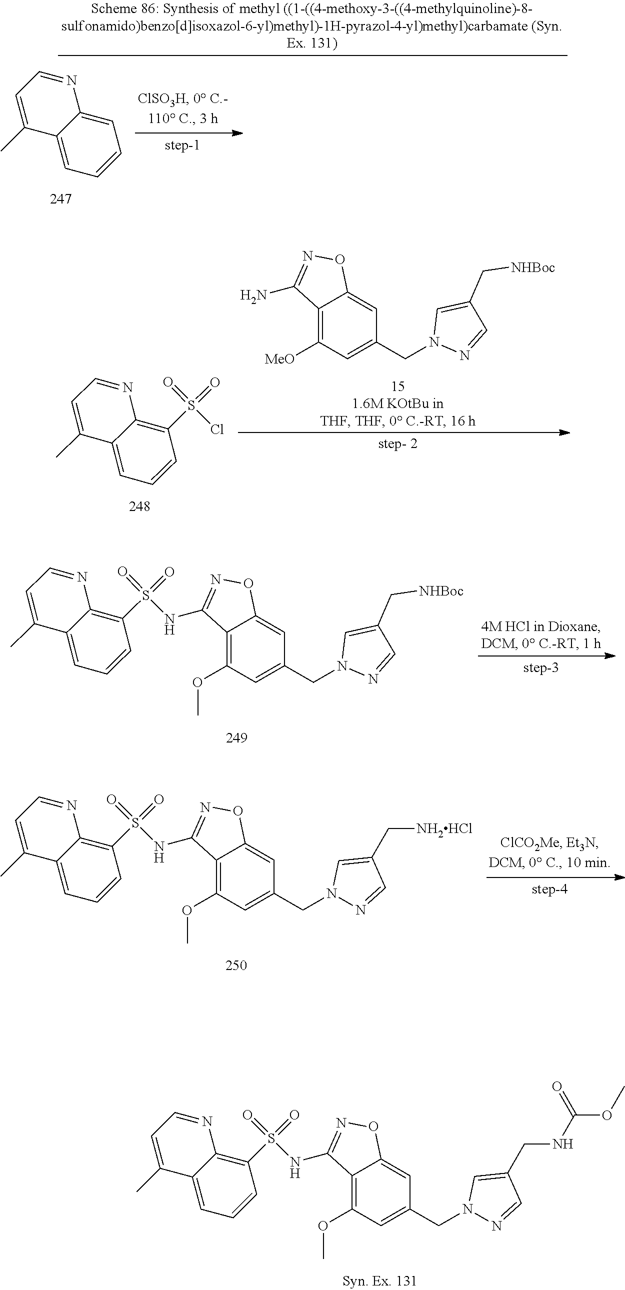

### Synthesis of 4-methylquinoline-8-sulfonyl chloride (248)

A solution of compound 247 (2 g, 13.967 mmol) in ClSO₃H (20 mL) was stirred at 110° C. for 3 h. After completion (monitored by TLC), the mixture was quenched with ice-cold water, extracted with EtOAc and washed with brine. The combined organic layers were dried over anhydrous Na₂SO₄, filtered and concentrated under reduced pressure to afford title compound 248 (2 g, 32.96%) as a brown solid. LCMS calculated for C10H8ClNO2S: 241; Found: 242.10 (M+1).

### Synthesis of tert-Butyl ((1-((4-methoxy-3-((4-methylquinoline)-8-sulfonamido)benzo[d]isoxazol-6-yl)methyl)-1H-pyrazol-4-yl)methyl)carbamate (249)

To a stirred solution of compound 15 (0.20 g, 0.535 mmol) in THF (20 mL) was added 1.6M KOtBu in THF (1 mL, 1.607 mmol) at 0° C. The reaction mixture was stirred for 5 min. followed by addition of compound 248 (0.23 g, 0.964 mmol). The reaction mixture was stirred at room temperature for 16 h. After completion (monitored by TLC), the mixture was quenched with aqueous citric acid and extracted with EtOAc. The combined organic layers were washed with brine, dried over anhydrous Na₂SO₄ and concentrated under reduced pressure to afford title compound 249 (0.35 g, 51.52%) as a yellow solid. LCMS calculated for C28H30N6O6S: 578.19; Found: 579.36 (M+1).

### Synthesis of N-(6-((4-(aminomethyl)-1H-pyrazol-1-yl)methyl)-4-methoxybenzo[d]isoxazol-3-yl)-4-methylquinoline-8-sulfonamide hydrochloride (250)

To a stirred solution of compound 249 (0.25 g, 0.432 mmol) in DCM (3 mL) was added 4M HCl in dioxane (2 mL) at 0° C. The reaction mixture was stirred at room temperature for 1 h. After completion (monitored by TLC), the mixture was concentrated under reduced pressure, washed with Et₂O and dried under reduced pressure to afford title compound 250 (0.15 g, 40.14%) as an off-white solid. LCMS calculated for C23H22N6O4S: 478.14; Found: 479.20 (M+1).

### Synthesis of methyl ((1-((4-methoxy-3-((4-methylquinoline)-8-sulfonamido)benzo[d]isoxazol-6-yl)methyl)-1H-pyrazol-4-yl)methyl)carbamate (Syn. Ex. 131)

To a stirred solution of compound 250 (0.10 g, 0.194 mmol) in DCM (10 mL) were added NEt₃ (0.08 mL, 0.582 mmol) and methyl chloroformate (0.02 g, 0.233 mmol) at 0° C. The reaction mixture was stirred at 0° C. for 10 min. After completion (monitored by TLC), the mixture was quenched with ice-cold water and extracted with EtOAc. The combined organic layers were dried over anhydrous Na₂SO₄ and concentrated under reduced pressure. The crude was purified by prep-HPLC to afford methyl ((1-((4-methoxy-3-((4-methylquinoline)-8-sulfonamido)benzol[d]isoxazol-6-yl)methyl)-1H-pyrazol-4-yl)methyl)carbamate (Syn. Ex. 131; 10 mg, 9.51%) as a white solid. (See analytical data in Table 1).

### Example 132

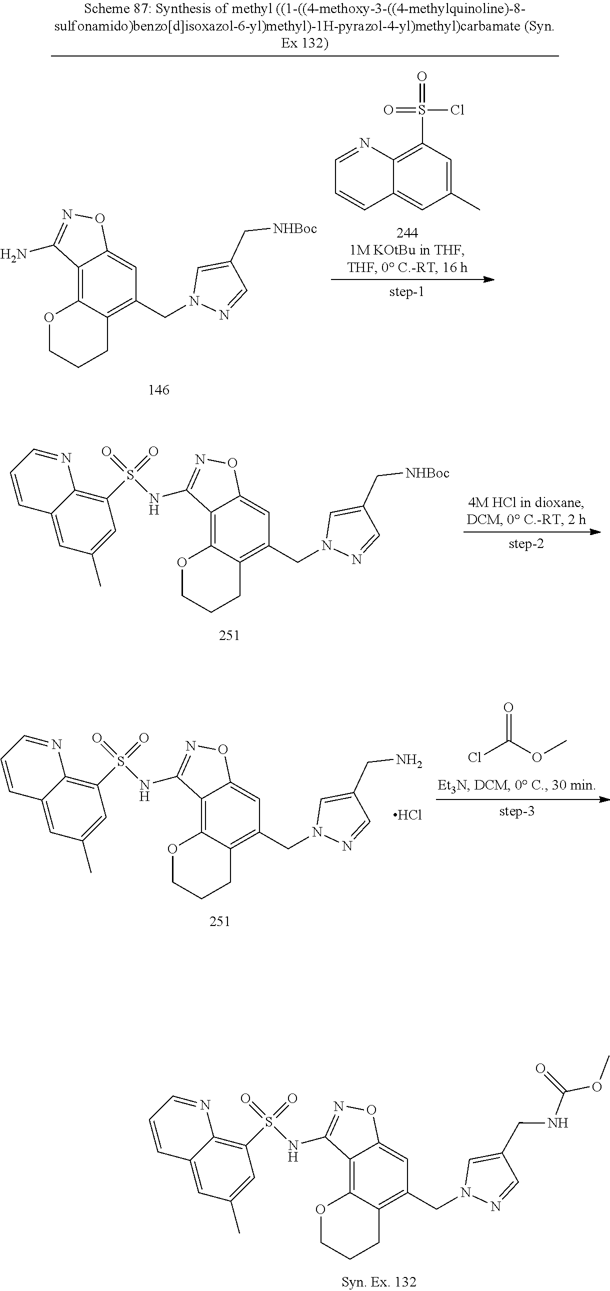

### Synthesis of tert-Butyl ((1-((9-((6-methylquinoline)-8-sulfonamido)-3,4-dihydro-2H-chromeno[8,7-d]isoxazol-5-yl)methyl)-1H-pyrazol-4-yl)methyl)carbamate (251)

To a stirred solution compound 146 (0.30 g, 0.751 mmol) in THF (10 mL) was added 1M KOtBu in THF (2.25 mL, 2.253 mmol) at 0° C. The reaction mixture was stirred at 0° C. for 10 min followed by addition of compound 244 (0.36 g, 1.502 mmol). The reaction mixture was stirred at room temperature for 16 h. After completion (monitored by TLC), the mixture was quenched with ice-cold water and extracted with EtOAc. The aqueous layer was acidified with 1N HCl to adjust the pH ˜6 and extracted with EtOAc. The combined organic layers were washed with brine, dried over anhydrous Na₂SO₄, filtered and concentrated under reduced pressure to afford title compound 251 (0.50 g, 44.03%) as a brown solid. LCMS calculated for C30H32N6O6S: 604.21; Found: 605.30 (M+1).

### Synthesis of N-(5-((4-(aminomethyl)-1H-pyrazol-1-yl)methyl)-3,4-dihydro-2H-chromeno[8,7-d]isoxazol-9-yl)-6-methylquinoline-8-sulfonamide hydrochloride (252)

To a stirred solution of compound 251 (0.50 g, 0.826 mmol) in DCM (mL) was added 4M HCl in dioxane (1.03 mL, 4.134 mmol) at 0° C. The reaction mixture was stirred at room temperature for 2 h. After completion (monitored by TLC), the mixture was concentrated under reduced pressure. The crude was washed with DCM, co-distilled with Et₂O and concentrated under reduced pressure to afford compound 252 (0.30 g, 46.27%) as an off-white solid. LCMS calculated for C25H24N6O4S: 504.16; Found: 505.20 (M+1)

### Synthesis of methyl ((1-((9-((6-methylquinoline)-8-sulfonamido)-3,4-dihydro-2H-chromeno[8,7-d]isoxazol-5-yl)methyl)-1H-pyrazol-4-yl)methyl)carbamate (Syn. Ex. 132)

To a stirred solution of compound 252 (0.40 g, 0.739 mmol) in DCM (6 mL) were added Et₃N (0.20 mL, 1.478 mmol) and methyl chloroformate (0.06 mL, 0.887 mmol) at 0° C. The reaction mixture was stirred at 0° C. for 30 min. After completion (monitored by TLC), the mixture was quenched with water and extracted with DCM. The combined organic layers were dried over anhydrous Na₂SO₄, filtered and concentrated under reduced pressure. The crude was purified by prep-HPLC to afford methyl ((1-((9-((6-methylquinoline)-8-sulfonamido)-3,4-dihydro-2H-chromeno[8,7-d]isoxazol-5-yl)methyl)-1H-pyrazol-4-yl)methyl)carbamate (Syn. Ex. 132; 10 mg, 2.35%) as an off-white solid. (See analytical data in Table 1).

### Example 133

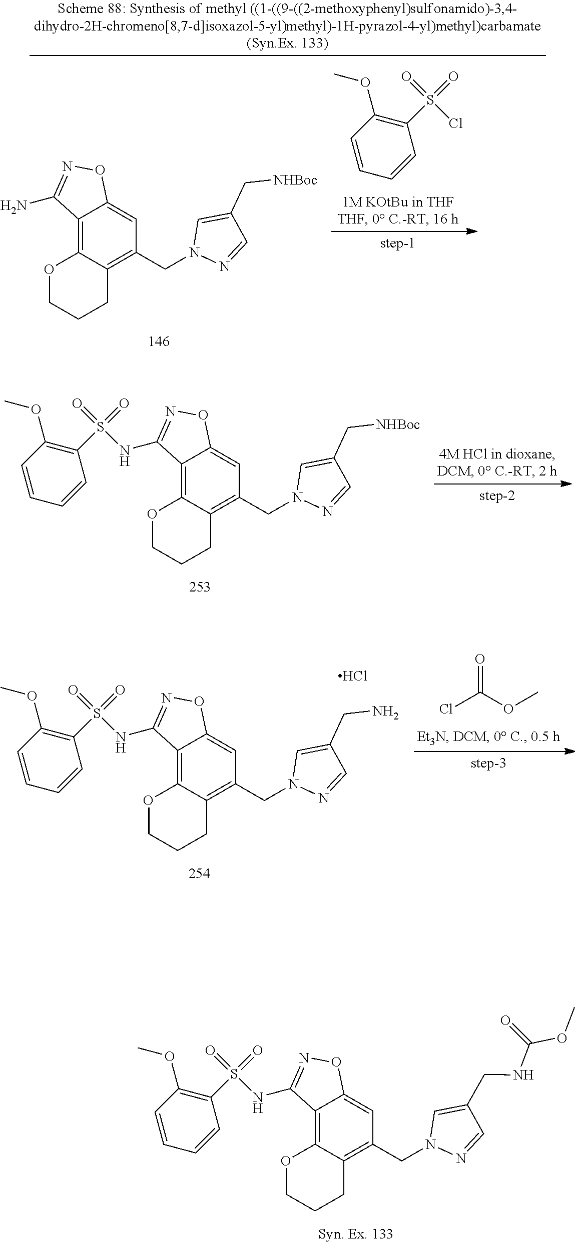

### Synthesis of tert-Butyl ((1-((9-((2-methoxyphenyl)sulfonamido)-3,4-dihydro-2H-chromeno[8,7-d]isoxazol-5-yl)methyl)-1H-pyrazol-4-yl)methyl)carbamate (253)

To a solution of compound 146 (0.15 g, 0.375 mmol) in THF (10 mL) was added 1M KOtBu in THF (1.12 mL, 1.126 mmol) at 0° C. The reaction mixture was stirred at 0° C. for 10 min. followed by addition of 2-methoxybenzenesulfonyl chloride (0.11 g, 0.563 mmol). The reaction mixture was stirred at room temperature for 16 h. After completion (monitored by TLC), the mixture was quenched with ice-cold water and extracted with EtOAc. The aqueous layer was acidified with 1N HCl to adjust the pH 6 and extracted with EtOAc. The combined organic layers were dried over anhydrous Na₂SO₄ and concentrated under reduced pressure to afford title compound 253 (0.20 g, 93.50%) as a brown solid. LCMS calculated for C27H31N5O7S: 569.19; Found: 568.20 (M−1).

### Synthesis of N-(5-((4-(aminomethyl)-1H-pyrazol-1-yl)methyl)-3,4-dihydro-2H-chromeno[8,7-d]isoxazol-9-yl)-2-methoxybenzenesulfonamide hydrochloride (254)

To a stirred solution of compound 253 (0.20 g, 0.351 mmol) in 1,4-dioxane (5 mL) and DCM (5 mL) was added 4M HCl in dioxane (0.43 mL, 1.755 mmol) at 0° C. The reaction mixture was stirred at room temperature for 2 h. After completion (monitored by TLC), the mixture was concentrated under reduced pressure, washed with DCM, co-distilled with Et₂O and dried under reduced pressure to afford title compound 254 (0.15 g, 48.98%) as an off-white solid. LCMS calculated for C22H23N5O5S: 469.14; Found: 468.20 (M−1).

### Synthesis of methyl ((1-((9-((2-methoxyphenyl)sulfonamido)-3,4-dihydro-2H-chromeno[8,7-d]isoxazol-5-yl)methyl)-1H-pyrazol-4-yl)methyl)carbamate (Syn. Ex. 133)

To a stirred solution of compound 254 (0.20 g, 0.395 mmol) in DCM (10 mL) were added triethylamine (0.11 mL, 0.790 mmol) and methyl chloroformate (0.04 g, 0.474 mmol) in DCM at 0° C. The reaction mixture was stirred at 0° C. for 30 min. After completion (monitored by TLC), the mixture was quenched with water and extracted with DCM. The combined organic layers were dried over anhydrous Na₂SO₄, filtered and concentrated under reduced pressure. The crude was purified by prep-HPLC to afford methyl ((1-((9-((2-methoxyphenyl)sulfonamido)-3,4-dihydro-2H-chromeno[8,7-d]isoxazol-5-yl)methyl)-1H-pyrazol-4-yl)methyl)carbamate (Syn. Ex. 133; 10 mg, 4.65%) as an off-white solid. (See analytical data in Table 1).

### Example 134

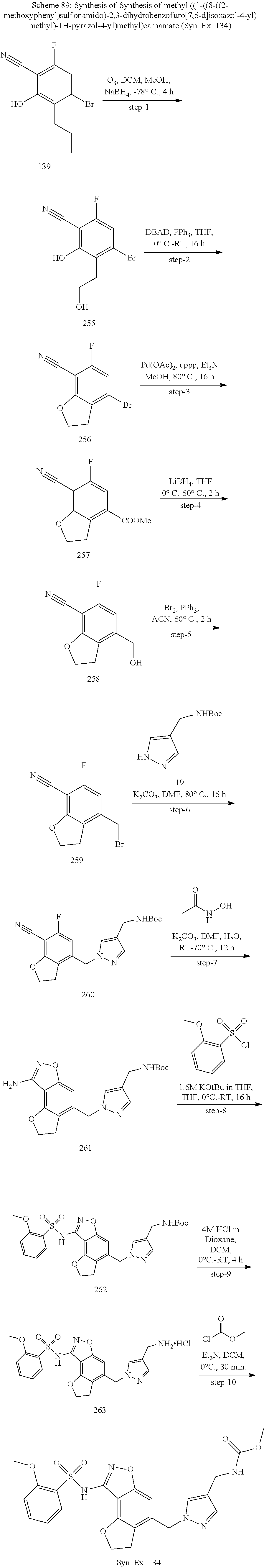

### Synthesis of 4-bromo-6-fluoro-2-hydroxy-3-(2-hydroxyethyl)benzonitrile (255)

To a stirred solution of compound 139 (2 g, 7.810 mmol) in DCM (10 mL) and MeOH (10 mL) ozone gas was purged for 5 min at −78° C. followed by addition of NaBH₄ (0.59 g, 15.620 mmol). The reaction was stirred at room temperature for 4 h. After completion (monitored by TLC), the mixture was quenched with 1N HCl, extracted with DCM and concentrated under reduced pressure. The crude was purified by combi-flash column chromatography using 25% ethyl acetate/Heptane to afford title compound 255 (0.90 g, 28.53%) as an off-white solid. LCMS calculated for C9H7BrFNO2: 258.96; Found: 258 (M−1).

### Synthesis of 4-bromo-6-fluoro-2,3-dihydrobenzofuran-7-carbonitrile (256)

To a solution of compound 255 (0.20 g, 0.769 mmol) in THF (10 mL) were added PPh₃ (0.30 g, 1.153 mmol) and DEAD (1.49 g, 7.306 mmol) at 0° C. The reaction mixture was stirred at room temperature for 16 h. After completion (monitored by TLC), the mixture was quenched with ice-cold water, extracted with EtOAc and concentrated under reduced pressure. The crude was purified by combi-flash column chromatography using 20% EtOAc/Heptane and concentrated under reduced pressure to afford title compound 256 (0.10 g, 53.73%) as an off-white solid. ¹H NMR (400 MHz, DMSO-d₆) δ 7.31 (d, J=9.54 Hz, 1H), 4.87 (t, J=8.71 Hz, 2H), 3.22 (br t, J=8.71 Hz, 2H).

### Synthesis of methyl 7-cyano-6-fluoro-2,3-dihydrobenzofuran-4-carboxylate (257)

To a stirred solution of compound 256 (0.30 g, 1.239 mmol) in MeOH (10 mL) was added Pd(OAc)₂ (0.01 g, 0.061 mmol), 1,3-Bis(diphenylphosphino)propane (0.05 g, 0.123 mmol) and triethylamine (0.34 mL, 2.478 mmol) and purged with N₂ for 10 min. The reaction mixture was stirred at 80° C. for 16 h under carbon monoxide gas (110 psi). After completion (monitored by TLC), the mixture was cooled at room temperature, filtered, and concentrated under reduced pressure. The crude was purified by combi-flash column chromatography using 20% EtOAc/Hexane to afford title compound 257 (0.15 g, 54.7%) as an off-white solid. ¹H NMR (401 MHz, DMSO-d₆) δ 7.29 (d, J=10.27 Hz, 1H), 4.86 (t, J=8.74 Hz, 2H), 3.87 (s, 3H), 3.50 (td, J=8.74, 1.71 Hz, 2H)

### Synthesis of 6-fluoro-4-(hydroxymethyl)-2,3-dihydrobenzofuran-7-carbonitrile (258)

To a stirred solution of compound 257 (3 g, 13.563 mmol) in THF (160 mL) was added LiBH₄ (0.62 g, 28.482 mmol) at 0° C. The reaction mixture was stirred at 60° C. for 2 h. After completion (monitored by TLC), the mixture was cooled at room temperature, quenched with water, extracted with EtOAc and concentrated under reduced pressure. The crude was purified by combi-flash column chromatography using 50% EtOAc/Heptane to afford title compound 258 (1.50 g, 55.70%) as an off-white solid. LCMS calculated for C10H8FNO2: 193.05; Found: 194.10 (M+1).

### Synthesis of 4-(bromomethyl)-6-fluoro-2,3-dihydrobenzofuran-7-carbonitrile (259)

To a stirred solution of compound 258 (0.50 g, 2.588 mmol) in acetonitrile (20 mL) were added triphenylphosphine (1.10 g, 4.141 mmol) and bromine (0.21 mL, 4.141 mmol). The reaction mixture was stirred at 60° C. for 2 h. After completion (monitored by TLC), the mixture was quenched with Na₂S2O₃, extracted with EtOAc, dried over anhydrous Na₂SO₄ and concentrated under reduced pressure. The crude was purified by combi-flash chromatography using 15% EtOAc/Heptane to afford title compound 259 (0.30 g, 45.26%) as a white solid. ¹H NMR (400 MHz, DMSO-d₆) δ ppm 7.03 (br d, J=10.37 Hz, 1H), 4.86 (br t, J=8.71 Hz, 2H), 4.64 (s, 2H), 3.26 (br t, J=8.71 Hz, 2H).

### Synthesis of tert-Butyl ((1-((7-cyano-6-fluoro-2,3-dihydrobenzofuran-4-yl)methyl)-1H-pyrazol-4-yl)methyl)carbamate (260)

To a stirred solution of compound 19 (1.10 g, 5.584 mmol) in DMF (30 mL) were added K₂CO₃ (1.78 g, 12.887 mmol) and compound 259 (1.10 g, 4.295 mmol). The reaction mixture was stirred at 80° C. for 16 h. After completion (monitored by TLC), the mixture was cooled at room temperature, quenched with water, extracted with EtOAc and concentrated under reduced pressure. The crude was purified by combi-flash column chromatography using 40% EtOAc/Hexane to afford title compound 260 (0.90 g, 37.75%) as an off-white solid. LCMS calculated for C19H21FN4O3: 372.16; Found: 373.30 (M+1).

### Synthesis of tert-Butyl ((1-((8-amino-2,3-dihydrobenzofuro[7,6-d]isoxazol-4-yl)methyl)-1H-pyrazol-4-yl)methyl)carbamate (261)

To a stirred solution of compound 260 (0.40 g, 1.074 mmol) in DMF (6 mL) and water (1 mL) were added K₂CO₃ (0.89 g, 6.445 mmol) and acetohydroxamic acid (0.48 g, 6.445 mmol) at room temperature. The reaction mixture was stirred at 70° C. for 12 h. After completion (monitored by TLC), the mixture was diluted with water and extracted with ethyl acetate. The combined organic layers were dried over anhydrous Na₂SO₄ and concentrated under reduced pressure. The crude was purified by combi-flash column chromatography using 60% EtOAc/Hexane to afford title compound 261 (0.25 g, 48.30%) as an off-white solid. LCMS calculated for C19H23N5O4: 385.18; Found: 386.28 (M+1).

### Synthesis of tert-Butyl ((1-((8-((2-methoxyphenyl)sulfonamido)-2,3-dihydrobenzofuro[7,6-d]isoxazol-4-yl)methyl)-1H-pyrazol-4-yl)methyl)carbamate (262)

To a stirred solution compound 261 (0.05 g, 0.129 mmol) in dry THF (5 mL) was added 1.6 M KOtBu in THF (0.24 mL, 0.389 mmol) at 0° C. The reaction mixture was stirred at 0° C. for 10 min followed by addition of 2-methoxybenzenesulfonyl chloride (0.05 g, 0.259 mmol). The reaction mixture was stirred at room temperature for 16 h. After completion (monitored by TLC), the mixture was quenched with ice-cold water, acidified with 1N HCl to adjust the pH 6 and extracted with EtOAc. The combined organic layers were washed with brine, dried over anhydrous Na₂SO₄, filtered and concentrated under reduced pressure to afford title compound 262 (0.04 g, 22.11%) as a brown solid. LCMS calculated for C26H29N5O7S: 555.18; Found: 556.38 (M+1).

### Synthesis of N-(4-((4-(aminomethyl)-1H-pyrazol-1-yl)methyl)-2,3-dihydrobenzofuro[7,6-d]isoxazol-8-yl)-2-methoxybenzenesulfonamide hydrochloride (263)

To a solution of compound 262 (40 mg, 0.071 mmol) in DCM (2 mL) was added 4M HCl in 1,4-dioxane (2 mL) at 0° C. The reaction mixture was stirred at room temperature for 4 h. After completion (monitored by TLC), the mixture was concentrated under reduced pressure triturated with Et₂O to afford title compound 263 (30 mg, 21.67%) as a brown solid. LCMS calculated for C21H21N5O5S: 455.13; Found: 457.20 (M+2)

### Synthesis of methyl ((1-((8-((2-methoxyphenyl)sulfonamido)-2,3-dihydrobenzofuro[7,6-d]isoxazol-4-yl)methyl)-1H-pyrazol-4-yl)methyl)carbamate (Syn. Ex. 134)

To a stirred solution of compound 263 (30 mg, 0.060 mmol) in DCM (3 mL) were added triethylamine (0.02 mL, 0.182 mmol) and methyl chloroformate (0.6 mg, 0.073 mmol) at 0° C. The reaction mixture was stirred at 0° C. for 30 min. After completion (monitored by TLC), the mixture was quenched with water and extracted with DCM. The combined organic layers were dried over anhydrous Na₂SO₄ and concentrated under reduced pressure. The crude was purified by prep-HPLC to afford methyl ((1-((8-((2-methoxyphenyl)sulfonamido)-2,3-dihydrobenzofuro[7,6-d]isoxazol-4-yl)methyl)-1H-pyrazol-4-yl)methyl)carbamate (Syn. Ex. 134, 3 mg, 9.42%) as an off-white solid. (See analytical data in Table 1).

### Example 135

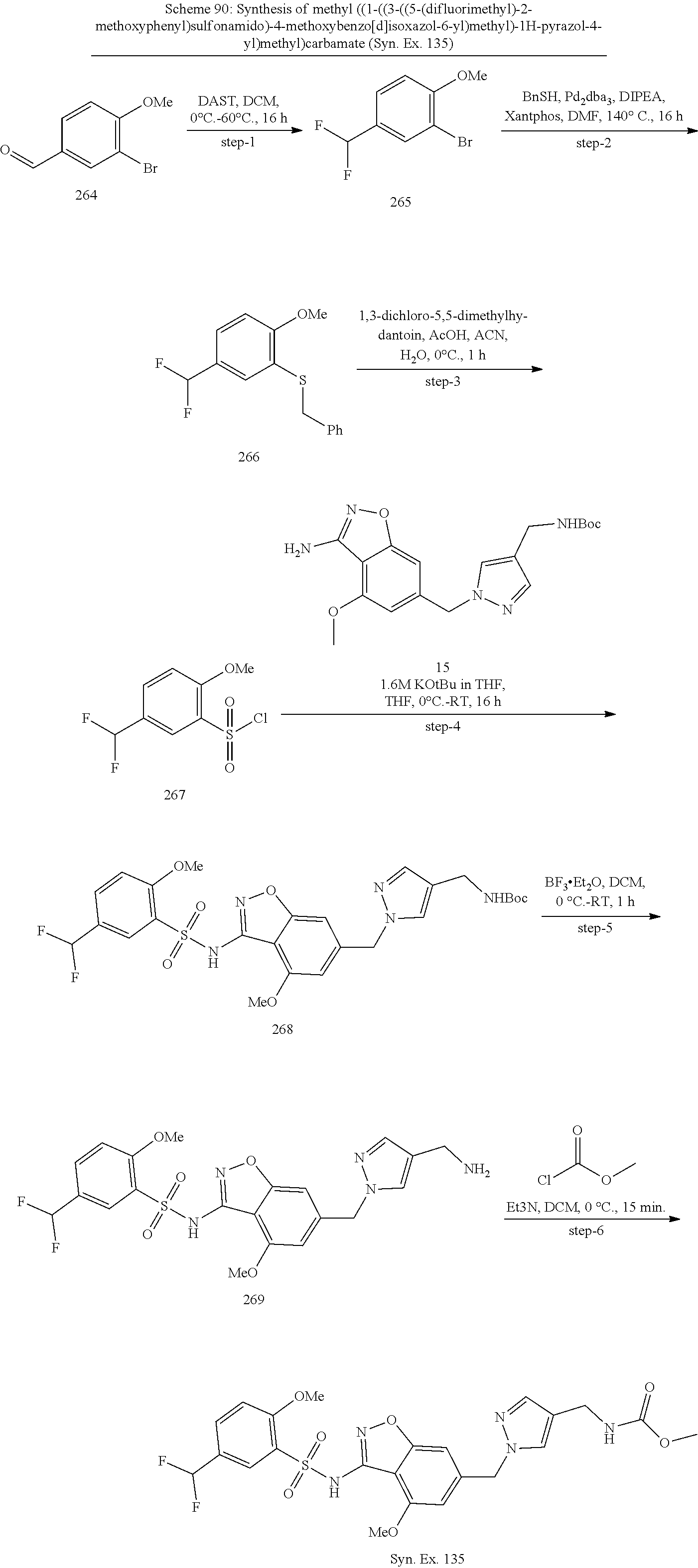

### Synthesis of 2-bromo-4-(difluoromethyl)-1-methoxybenzene (265)

To a stirred solution of compound 264 (5 g, 23.250 mmol) in DCM (50 mL) was added DAST (11.83 g, 69.751 mmol) at 0° C. The reaction mixture was stirred at 60° C. for 16 h. After completion (monitored by TLC), the mixture was quenched with water and extracted with DCM. The combined organic layers were dried over anhydrous Na₂SO₄ and concentrated under reduced pressure. The crude was purified by combi-flash column chromatography using 7-9% EtOAc/Hexane to afford title compound 265 (4.50 g, 79.20%) as a yellow liquid. TLC: 20% EtOAc/Hexane (R_{f}: 0.6); ¹H NMR (400 MHz, DMSO-d₆) δ ppm 7.75-7.80 (m, 1H), 7.58 (dt, J=8.60, 0.89 Hz, 1H), 7.24 (d, J=8.63 Hz, 1H), 6.79-7.15 (m, 1H), 3.90 (s, 3H)

### Synthesis of benzyl(5-(difluoromethyl)-2-methoxyphenyl)sulfane (266)

To a stirred solution of compound 265 (0.50 g, 2.109 mmol) in DMF (5 mL) were added DIPEA (1.10 mL, 6.328 mmol) and XantPhos (0.25 g, 0.421 mmol). The reaction mixture was degassed with N₂ for 10 min followed by addition of benzyl mercaptan (1.04 g, 8.437 mmol) and Pd₂(dba)₃ (0.19 g, 0.210 mmol). The reaction mixture was stirred at 140° C. for 16 h. After completion (monitored by TLC), the mixture was quenched with water and extracted with EtOAc. The combined organic layers were dried over anhydrous Na₂SO₄ and concentrated under reduced pressure. The crude was purified by combi-flash column chromatography using 0-2% EtOAc/Hexane to afford title compound 266 (0.30 g, 33.49%) as a yellow semisolid; ¹H NMR (400 MHz, DMSO-d₆) δ ppm 7.44 (s, 1H), 7.35-7.40 (m, 3 H), 7.21-7.34 (m, 3H), 7.04-7.12 (m, 1H), 6.91 (s, 1H), 4.20 (s, 2H), 3.86 (s, 3H)

### Synthesis of 5-(difluoromethyl)-2-methoxybenzenesulfonyl chloride (267)

To a stirred solution of compound 266 (0.30 g, 1.070 mmol) in ACN (6 mL), water (0.10 mL) and AcOH (0.20 mL) was added 1,3-dichloro-5,5-dimethylhydantoin (0.43 g, 2.141 mmol) at 0° C. The reaction mixture was stirred at 0° C. for 1 h. After completion (monitored by TLC), the mixture was quenched with water and extracted with EtOAc. The combined organic layers were dried over anhydrous Na₂SO₄ and concentrated under reduced pressure to afford title compound 267 (0.20 g, 40.05%) as a yellow semiliquid; ¹H NMR (400 MHz, DMSO-d₆) δ ppm 7.76-7.92 (m, 1H), 7.50 (br d, J=8.33 Hz, 1H), 7.19-7.37 (m, 1 H), 6.74-7.13 (m, 1H), 3.90 (s, 3H).

### Synthesis of tert-Butyl ((1-((3-((5-(difluoromethyl)-2-methoxyphenyl)sulfonamido)-4-methoxybenzo[d]isoxazol-6-yl)methyl)-1H-pyrazol-4-yl)methyl)carbamate (268)

To a stirred solution of compound 15 (0.15 g, 0.401 mmol) in THF (5 mL) was added 1.6M KOtBu in THF (0.75 mL, 1.205 mmol) at 0° C. The reaction mixture was stirred at 0° C. for 10 min followed by addition of compound 267 (0.20 g, 0.803 mmol) in THF. The reaction mixture was stirred at room temperature for 16 h. After completion (monitored by TLC), the mixture was quenched with water and extracted with EtOAc. The organic layer was dried over anhydrous Na₂SO₄, filtered and concentrated under reduced pressure. The crude was purified by combi-flash column chromatography using 70-72% EtOAc/Pet ether to afford title compound 268 (0.12 g, 26.67%) as an off-white solid. TLC: 80% EtOAc/Hexane (R_{f}: 0.4). LCMS calculated for C26H29F2N5O7S: 593.18; Found: 594.30 (M+1).

### Synthesis of N-(6-((4-(aminomethyl)-1H-pyrazol-1-yl)methyl)-4-methoxybenzo[d]isoxazol-3-yl)-5-(difluoromethyl)-2-methoxybenzenesulfonamide (269)

To a solution of compound 268 (0.12 g, 0.202 mmol) in DCM (2 mL) was added BF₃·Et₂O (0.03 mL, 0.303 mmol) at 0° C. The reaction mixture was stirred at room temperature for 1 h. After completion (monitored by TLC), the mixture was concentrated under reduced pressure. The residue was washed Et₂O and concentrated under reduced pressure to afford title compound 269 (0.10 g, 53.13%) as an off-white solid. LCMS calculated for C21H21F2N5O5S: 493.12; Found: 492.20 (M−1).

### Synthesis of methyl ((1-((3-((5-(difluoromethyl)-2-methoxyphenyl)sulfonamido)-4-methoxybenzo[d]isoxazol-6-yl)methyl)-1H-pyrazol-4-yl)methyl)carbamate (Syn. Ex. 135)

To a solution of compound 269 (0.13 g, 0.263 mmol) in DCM (5 mL) were added NEt₃ (0.11 mL, 0.790 mmol) in DCM (0.20 mL) and methyl chloroformate (0.02 mL, 0.316 mmol) in DCM (0.20 mL) at 0° C. The reaction mixture was stirred at 0° C. for 15 min. After completion (monitored by TLC), the mixture was quenched with water and extracted with DCM. The organic layer was dried over anhydrous Na₂SO₄, filtered, and concentrated under reduced pressure. The crude was purified by prep-HPLC to afford methyl ((1-((3-((5-(difluoromethyl)-2-methoxyphenyl)sulfonamido)-4-methoxybenzo[d]isoxazol-6-yl)methyl)-1H-pyrazol-4-yl)methyl)carbamate (Syn. Ex. 135; 5.00 mg, 3.34% yield) as an off-white solid. TLC: 5% MeOH/DCM (R_{f}: 0.4). (See analytical data in Table 1).

### Example 136

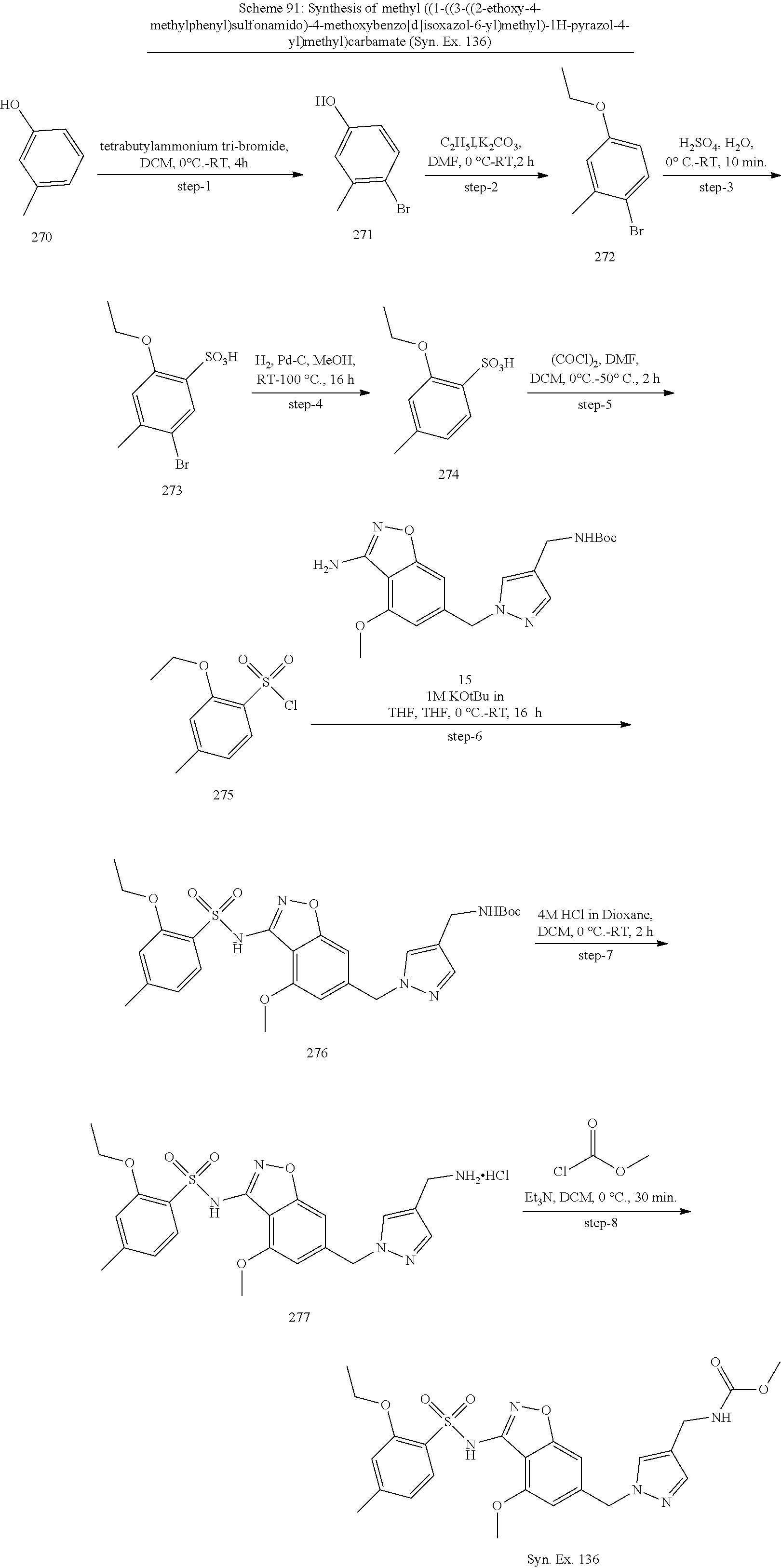

### Synthesis of 4-bromo-3-methylphenol (271)

To a stirred solution of m-cresol 270 (3 g, 27.742 mmol) in DCM (30 mL) was added tetrabutylammonium tribromide (13.37 g, 27.742 mmol) at 0° C. The reaction mixture was stirred at room temperature for 4 h. After completion (monitored by TLC), the mixture was quenched with ice-cold water and extracted with DCM. The combined organic layers were dried over anhydrous Na₂SO₄, filtered and concentrated under reduced pressure. The crude was purified by combi-flash column chromatography using 20-30% of EtOAc/Heptane to afford title compound 271 (4.50 g, 64%) as a white semisolid. TLC: 30% EtOAc/Heptane (R_{f}: 0.3). LCMS calculated for C7H7BrO: 185.97; Found: 185.20 (M−1).

### Synthesis of 1-bromo-4-ethoxy-2-methylbenzene (272)

To a stirred solution of compound 271 (4.50 g, 24.059 mmol) in DMF (40 mL) was added K₂CO₃ (4.76 g, 48.118 mmol) at 0° C. The reaction mixture was stirred at 0° C. for 5 min followed by addition of ethyl iodide (2.96 mL, 36.088 mmol). The reaction mixture was stirred at room temperature for 2 h. After completion (monitored by TLC), the mixture was quenched with ice-cold water and extracted with EtOAc. The combined organic layers were washed with cold water, dried over anhydrous Na₂SO₄, filtered, and concentrated under reduced pressure to afford title compound 272 (3.50 g, 54.78%) as a colourless liquid. TLC: 20% EtOAc/Heptane (R_{f}: 0.8); ¹H NMR (400 MHz, DMSO-d₆) δ ppm 7.42 (d, J=8.71 Hz, 1 H), 6.94 (br s, 1H), 6.71 (br dd, J=8.71, 2.07 Hz, 1H), 3.99 (q, J=6.77 Hz, 2H), 2.29 (s, 3 H), 1.30 (t, J=7.05 Hz, 3H)

### Synthesis of 5-bromo-2-ethoxy-4-methylbenzenesulfonic acid (273)

A solution of compound 272 (2 g, 9.298 mmol) in H₂SO₄ (20 mL) was stirred at room temperature for 10 min. The reaction was quenched by addition of water at 0° C. The solid residue was filtered, washed with Heptane and dried under reduced pressure to afford title compound 273 (2.30 g, 81.29%) as a pale-brown solid. LCMS calculated for C9H11BrO4S: 293.96; Found: 293.10 (M−1).

### Synthesis of 2-ethoxy-4-methylbenzenesulfonic acid (274)

To a stirred solution of compound 273 (2.30 g, 7.792 mmol) in MeOH (50 mL) was added 10% Pd—C(1.49 g, 1.402 mmol) at room temperature. The reaction mixture was stirred at 100° C. for 16 h under H₂ atmosphere (100 psi). After completion (monitored by TLC), the mixture was filtered through celite pad, washed with MeOH and concentrated under reduced pressure. The crude was washed with ether and dried under reduced pressure to afford title compound 274 (1.60 g, 93.05%) as brown semisolid. TLC: 20% MeOH/DCM (R_{f}: 0.4). LCMS calculated for C9H12O4S: 216.05; Found: 215.30 (M−1).

### Synthesis of 2-ethoxy-4-methylbenzenesulfonyl chloride (275)

To a stirred solution of compound 274 (0.40 g, 1.849 mmol) in DCM (4 mL) were added oxalyl chloride (3.99 mL, 45.706 mmol) and DMF (0.04 mL) at 0° C. The reaction mixture was stirred at 50° C. for 2 h under N₂. After completion (monitored by TLC), the mixture was quenched with ice-cold water and extracted with EtOAc. The organic layer was dried over anhydrous Na₂SO₄, filtered and concentrated under reduced pressure to afford title compound 275 (0.42 g, 96.75%) as a colorless liquid. TLC: 50% EtOAc/Heptane (R_{f}: 0.8); ¹H NMR (400 MHz, DMSO-d₆) δ ppm 7.54 (br d, J=7.46 Hz, 1H), 6.75 (s, 1H), 6.63 (br d, J=7.46 Hz, 1H), 3.98-4.04 (m, 2H), 2.27 (s, 3H), 1.29 (br t, J=6.84 Hz, 3H)

### Synthesis of tert-Butyl ((1-((3-((2-ethoxy-4-methylphenyl)sulfonamido)-4-methoxybenzo[d]isoxazol-6-yl)methyl)-1H-pyrazol-4-yl)methyl)carbamate (276)

To a stirred solution of compound 15 (0.30 g, 0.803 mmol) in THF (10 mL) was added 1M KOtBu in THF (2.41 mL, 2.410 mmol) at 0° C. The reaction mixture was stirred for 5 min. followed by addition of compound 275 (0.37 g, 1.607 mmol). The reaction mixture was stirred at room temperature for 16 h. After completion (monitored by TLC), the mixture was quenched with ice-cold water and extracted with EtOAc. The organic layer was dried over anhydrous Na₂SO₄, filtered, concentrated under reduced pressure. The solid residue was washed with ether and dried under reduced pressure to afford title compound 276 (0.32 g, 29.26%) as pale-yellow semisolid. TLC:EtOAc (R_{f}: 0.2). LCMS calculated for C27H33N5O7S: 571.21; Found: 570.20 (M−1)

### Synthesis of N-(6-((4-(aminomethyl)-1H-pyrazol-1-yl)methyl)-4-methoxybenzo[d]isoxazol-3-yl)-2-ethoxy-4-methylbenzenesulfonamide hydrochloride (277)

To a stirred solution of compound 276 (0.30 g, 0.524 mmol) in DCM (5 mL) was added 4M HCl in dioxane (1.31 mL, 5.248 mmol) at 0° C. The reaction mixture was stirred at room temperature for 2 h. After completion (monitored by TLC), the mixture was concentrated under reduced pressure. The crude was triturated with ether and dried under reduced pressure to afford title compound 277 (0.25 g, 52.52%) as pale-yellow solid. LCMS calculated for C22H25N5O5S: 471.16; Found: 470.30 (M−1).

### Synthesis of methyl ((1-((3-((2-ethoxy-4-methylphenyl)sulfonamido)-4-methoxybenzo[d]isoxazol-6-yl)methyl)-1H-pyrazol-4-yl)methyl)carbamate (Syn. Ex. 136)

To a stirred solution of compound 277 (0.16 g, 0.314 mmol) in DCM (3 mL) was added Et₃N (0.08 mL, 0.629 mmol) and methyl chloroformate (0.03 g, 0.314 mmol) in DCM (1 mL) at 0° C. The reaction mixture was stirred at 0° C. for 30 min. After completion (monitored by TLC), the mixture was quenched with ice-cold water (10 mL) and extracted with DCM. The organic layer was dried over anhydrous Na₂SO₄, filtered, and concentrated under reduced pressure. The crude was purified by prep-HPLC to afford methyl ((1-((3-((2-ethoxy-4-methylphenyl)sulfonamido)-4-methoxybenzo[d]isoxazol-6-yl)methyl)-1H-pyrazol-4-yl)methyl)carbamate (Syn. Ex. 136; 5.00 mg, 2.96%) as an off-white solid. TLC: 10% MeOH/DCM (R_{f}: 0.7). (See analytical data in Table 1).

### Example 137

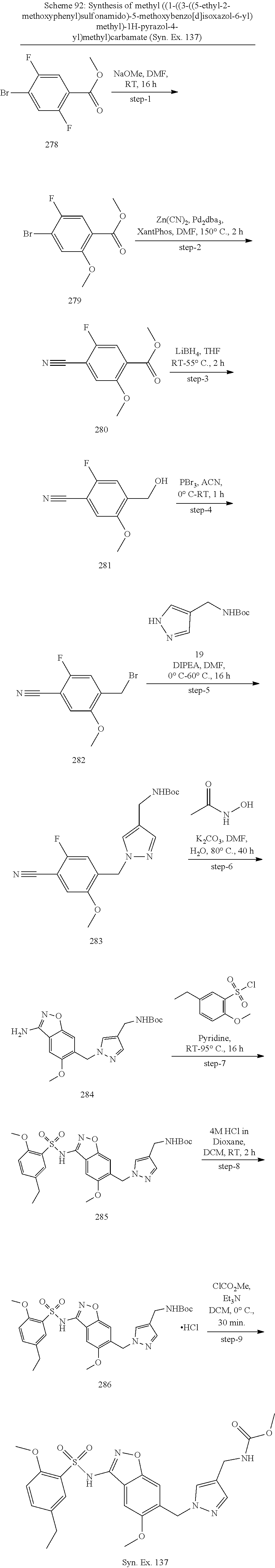

### Synthesis of methyl 4-bromo-5-fluoro-2-methoxybenzoate (279)

To a stirred solution of compound 278 (2 g, 8 mmol) in DMF (20 mL) was added NaOMe (0.54 g, 9.60 mmol) at room temperature. The reaction mixture was stirred at room temperature for 16 h. After completion (monitored by TLC), the mixture was quenched with ice-cold water and extracted with EtOAc. The combined organic layers were dried over anhydrous Na₂SO₄, filtered, and concentrated under reduced pressure. The crude was purified by combi-flash chromatography to afford title compound 279 (1 g, 47%) as a white solid. TLC: 10% EtOAc/Heptane (R_{f}: 0.4). LCMS calculated for C9H8BrFO3: 261.96; Found: 263 (M+1).

### Synthesis of methyl 4-cyano-5-fluoro-2-methoxybenzoate (280)

To a stirred solution of compound 279 (50 mg, 0.190 mmol) in DMF (2 mL) was added Zn(CN)₂ (g, 0.285 mmol). The reaction mixture was degassed with N₂ for 5 min followed by addition of XantPhos (0.01 g, 0.019 mmol) and Pd₂(dba)₃ (0.0087 g, 0.009 mmol). The reaction mixture was stirred at 150° C. for 2 h. After completion (monitored by TLC), the mixture was quenched with ice-cold water and extracted with EtOAc. The combined organic layers were dried over anhydrous Na₂SO₄, filtered and concentrated under reduced pressure. The crude was purified by combi-flash chromatography to afford title compound 280 (26 mg, 64.66%) as a brown solid. TLC: 30% EtOAc/Heptane; ¹H NMR (400 MHz, DMSO-d₆) δ 7.72-7.77 (m, 2H) 3.84 (d, J=13.13 Hz, 6H)

### Synthesis of 2-fluoro-4-(hydroxymethyl)-5-methoxybenzonitrile (281)

To a stirred solution of compound 280 (100 mg, 0.478 mmol) in THF (4 mL) was added LiBH₄ (2 mol/L) in THF (0.72 mL, 1.434 mmol) at room temperature. The reaction mixture was stirred at 55° C. for 2 h. After completion (monitored by TLC), the mixture was quenched with ice-cold saturated aq. NH₄Cl and extracted with EtOAc. The combined organic layers were dried over anhydrous Na₂SO₄, filtered and concentrated under reduced pressure. The crude was triturated with DCM/Heptane (9:1 ratio) to afford title compound 281 (82 mg, 91.59%) as a white solid. TLC: 40% EtOAc/Heptane, (R_{f}: 0.45); ¹H NMR (401 MHz, DMSO-d₆) δ 7.47 (d, J=5.14 Hz, 1H) 7.40 (d, J=9.90 Hz, 1H) 5.50 (t, J=5.56 Hz, 1 H) 4.49-4.54 (m, 2H) 3.83 (s, 3H)

### Synthesis of 4-(bromomethyl)-2-fluoro-5-methoxybenzonitrile (282)

To a solution of compound 281 (80 mg, 0.441 mmol) in CH₃CN (3 mL) was added PBr₃ (0.08 mL, 0.883 mmol) at 0° C. The reaction mixture was warm to room temperature and stirred for 1 h. After completion (monitored by TLC), the mixture was quenched with saturated aqueous NaHCO₃ and extracted with EtOAc. The combined organic layers were dried over anhydrous Na₂SO₄, filtered and concentrated under reduced pressure. The crude was purified by combi-flash chromatography using 6-10% EtOAc/Heptane to afford title compound 282 (60 mg, 54.82%) as a white solid. TLC: 10% EtOAc/Heptane, (R_{f}: 0.4); ¹H NMR (400 MHz, DMSO-d₆) δ ppm 7.66 (d, J=9.65 Hz, 1H) 7.60 (d, J=5.26 Hz, 1H) 4.62 (s, 2H) 3.90 (s, 3H)

### Synthesis of tert-Butyl ((1-(4-cyano-5-fluoro-2-methoxybenzyl)-1H-pyrazol-4-yl)methyl)carbamate (283)

To a stirred solution of compound 19 (1.20 g, 5.90 mmol) in DMF (15 mL) were added DIPEA (2.60 mL, 15 mmol) and compound 282 (1.20 g, 4.90 mmol) at 0° C. The reaction mixture was stirred at 60° C. for 16 h. After completion (monitored by TLC), the mixture was quenched with ice-cold water and extracted with EtOAc. The combined organic layers were dried over anhydrous Na₂SO₄, filtered and concentrated under reduced pressure. The crude was purified by combi-flash chromatography using 40-52% EtOAc/Heptane to afford title compound 283 (0.90 g, 46%) as a brown gummy oil. TLC: 50% EtOAc/Heptane, (R_{f}: 0.4). LCMS calculated for C18H21FN4O3: 360.16; Found: 361.10 (M+1).

### Synthesis of tert-Butyl ((1-((3-amino-5-methoxybenzo[d]isoxazol-6-yl)methyl)-1H-pyrazol-4-yl)methyl)carbamate (284)

To a stirred solution of compound 283 (0.10 g, 0.277 mmol) in DMF (3 mL) and water (0.60 mL) were added N-hydroxyacetamide (0.062 g, 0.832 mmol) and K₂CO₃ (0.19 g, 1.387 mmol). The reaction mixture was stirred at 80° C. for 40 h. After completion (monitored by TLC), the mixture was diluted with water and extracted with EtOAc. The combined organic layers were dried over anhydrous Na₂SO₄ and concentrated under reduced pressure. The crude was triturated with Et₂O and dried to afford title compound 284 (0.03 g, 20.61%) as a brown solid. LCMS calculated for C18H23N5O4: 373.18; Found: 374.20 (M+1)

### Synthesis of tert-Butyl ((1-((3-((5-ethyl-2-methoxyphenyl)sulfonamido)-5-methoxybenzo[d]isoxazol-6-yl)methyl)-1H-pyrazol-4-yl)methyl)carbamate (285)

To a solution of compound 284 (0.25 g, 0.669 mmol) in Pyridine (3 mL) was added 5-ethyl-2-methoxybenzenesulfonyl chloride (0.18 g, 0.80 mmol) at room temperature. The reaction mixture was stirred at 95° C. for 16 h. After completion (monitored by TLC), the mixture was quenched with water and extracted with EtOAc. The combined organic layers were dried over anhydrous Na₂SO₄ and concentrated under reduced pressure. The crude was purified by combi-flash chromatography using 50-60% EtOAc/Heptane to afford title compound 285 (0.05 g, 12.93%) as a brown solid. LCMS calculated for C27H33N5O7S: 571.21; Found: 572.40 (M+1).

### Synthesis of N-(6-((4-(aminomethyl)-1H-pyrazol-1-yl)methyl)-5-methoxybenzo[d]isoxazol-3-yl)-5-ethyl-2-methoxybenzenesulfonamide hydrochloride (286)

To a stirred solution of compound 285 (0.095 g, 0.166 mmol) in DCM (4 mL) was added 4M HCl in 1,4-Dioxane (0.20 mL, 0.831 mmol) at 0° C. The reaction mixture was stirred at room temperature for 2 h. After completion (monitored by TLC), the mixture was concentrated under reduced pressure. The crude was triturated with Et₂O to afford title compound 286 (0.08 g, 83.07%) as a brown solid. LCMS calculated for C22H25N5O5S: 471.16; Found: 470.30 (M−1).

### Synthesis of methyl ((1-((3-((5-ethyl-2-methoxyphenyl)sulfonamido)-5-methoxybenzo[d]isoxazol-6-yl)methyl)-1H-pyrazol-4-yl)methyl)carbamate (Syn. Ex. 137)

To a stirred solution compound 286 (0.07 g, 0.148 mmol) in DCM (5 mL) was added Et₃N (0.066 mL, 0.445 mmol) and methyl chloroformate (0.012 mL, 0.163 mmol) at 0° C. The reaction mixture was stirred at 0° C. for 30 min. After completion (monitored by TLC), the mixture was quenched with ice-cold water and extracted with DCM. The combined organic layers were dried over anhydrous Na₂SO₄, filtered, and concentrated under reduced pressure. The crude was purified by prep-HPLC to afford title compound (Syn. Ex. 137; 7.00 mg, 8.82%) as an off-white solid. (See analytical data in Table 1).

### Example 138

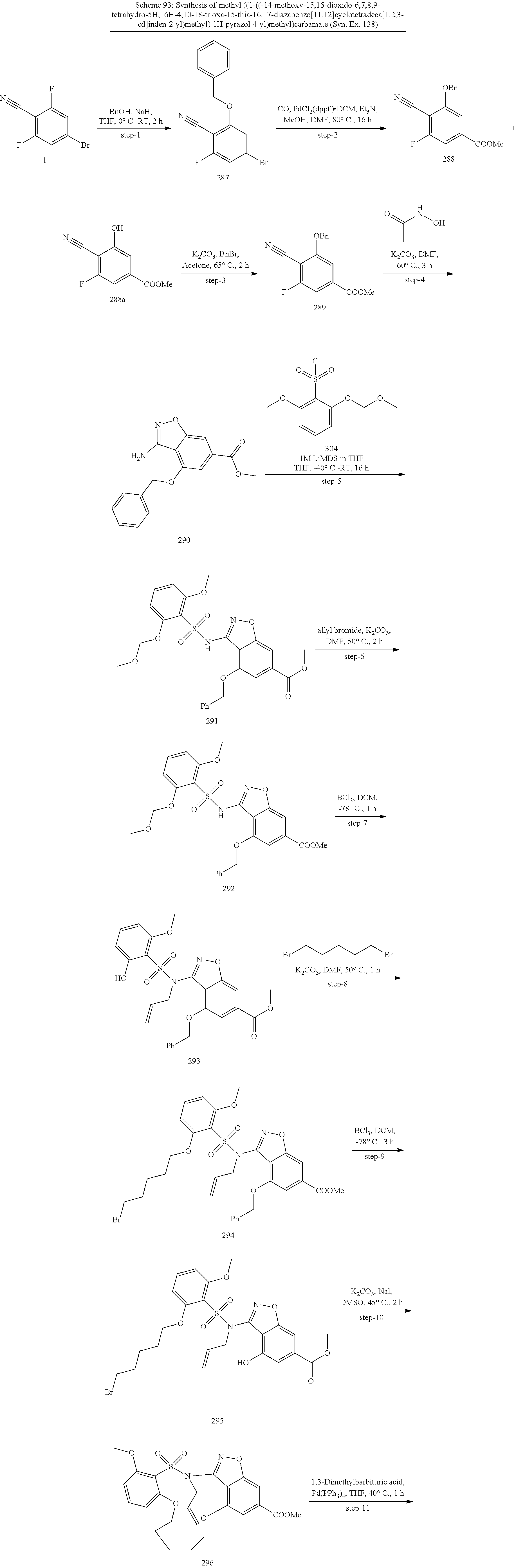

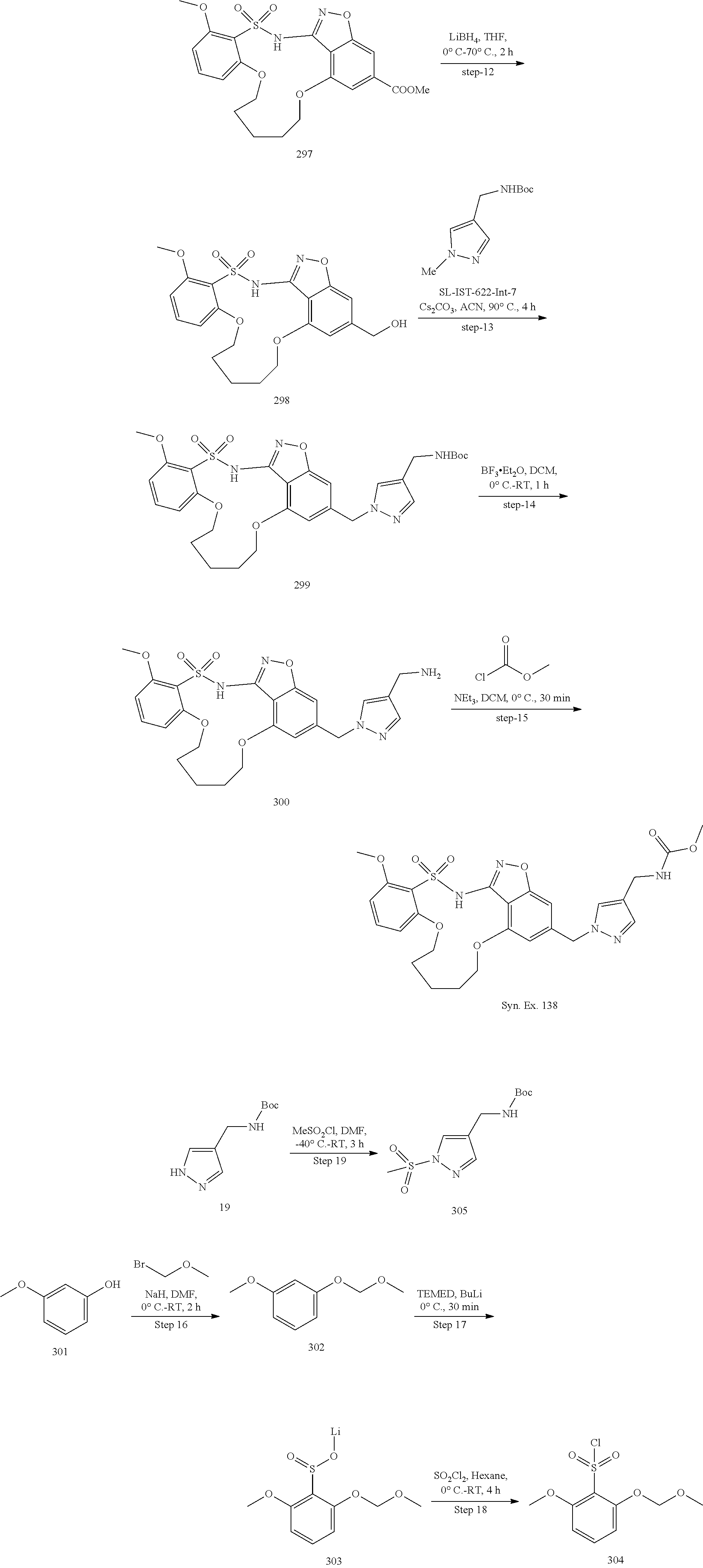

### Synthesis of 2-(benzyloxy)-4-bromo-6-fluorobenzonitrile (287)

To a stirred solution of 60% NaH in mineral oil (2.20 g, 55.05 mmol) in THF (200 mL) was added BnOH (4.75 mL, 45.87 mmol) in THF (5 mL) at 0° C. The reaction mixture was stirred at room temperature for 1 h followed by addition of compound 1 (10 g, 45.87 mmol) in THF (50 mL) at 0° C. The reaction mixture was stirred at room temperature for 2 h. After completion (monitored by TLC), the mixture was quenched with ice-cold water and extracted with EtOAc. The combined organic layers were washed with brine, dried over anhydrous Na₂SO₄, filtered, and concentrated under reduced pressure. The crude was purified by combi-flash chromatography using 12-23% EtOAc/Heptane to afford title compound 287 (8 g, 57%) as a white solid; ¹H NMR (400 MHz, DMSO-d₆) δ ppm 7.30-7.58 (m, 7H), 5.35 (s, 2H)

### Synthesis of methyl 3-(benzyloxy)-4-cyano-5-fluorobenzoate (288) and methyl 4-cyano-3-fluoro-5-hydroxybenzoate (288a)

A solution of compound 287 (10.50 g, 34.30 mmol) in MeOH (150 mL) and DMF (150 mL) was degassed with nitrogen for 10 min followed by addition of PdCl₂(dppf)·DCM (2.83 g, 3.43 mmol) and Et₃N (14.40 mL, 103 mmol). The reaction mixture was stirred at 80° C. for 16 h under CO atmosphere (80 psi). After completion (monitored by TLC), the mixture was cooled at room temperature, filtered, and concentrated under reduced pressure to afford the mixture of compound 288 and compound 288a (17 g, 88.70%) as a brown gummy oil.

### Synthesis of methyl 3-(benzyloxy)-4-cyano-5-fluorobenzoate (289)

To a stirred solution of mixture of compound 288 and compound 288a (16 g, 33.30 mmol) in acetone (160 mL) were added K₂CO₃ (6.90 g, 50 mmol) and BnBr (2.37 mL, 20 mmol). The reaction mixture was stirred 65° C. for 2 h. After completion (monitored by TLC), the mixture was quenched with ice-cold water and was extracted with EtOAc. The combined organic layers were washed with brine, dried over anhydrous Na₂SO₄, filtered, and concentrated under reduced pressure. The crude was purified by combi-flash chromatography using 12-16% EtOAc/Heptane to afford title compound 289 (10 g, 99.50%) as a brown solid; ¹H NMR (400 MHz, DMSO-d₆) δ ppm 7.65 (s, 1H), 7.35-7.57 (m, 6H), 5.42 (s, 2H), 3.91 (s, 3H)

### Synthesis of methyl 3-amino-4-(benzyloxy)benzo[d]isoxazole-6-carboxylate (290)

To a solution of compound 289 (5 g, 18 mmol) in DMF (75 mL) were added N-hydroxyacetamide (4 g, 53 mmol) and K₂CO₃ (12 g, 88 mmol). The reaction mixture was stirred 60° C. for 3 h. After completion (monitored by TLC), the mixture was quenched with ice-cold water and extracted with EtOAc. The combined organic layers were washed with brine, dried over anhydrous Na₂SO₄, filtered, and concentrated under reduced pressure. The crude was washed with pentane, IPA and filtered to afford title compound (290) (2.30 g, 40%) as a white solid. LCMS calculated for C16H₁₄N₂O₄: 298.10; Found: 299.10 (M+1).

### Synthesis of methyl 4-(benzyloxy)-3-((2-methoxy-6-(methoxymethoxy)phenyl)sulfonamido)benzo[d]isoxazole-6-carboxylate (291)

To a stirred solution of compound 290 (2 g, 6.70 mmol) in THF (80 mL) was added 1M LiHMDS in THF (20 mL, 20 mmol) at −40° C. The reaction mixture was stirred at −40° C. for 1 h followed by addition of compound 304 (3.60 g, 13 mmol) in THF (2 mL). The reaction mixture was stirred at room temperature for 16 h. After completion (monitored by TLC), the mixture was quenched with ice-cold water and extracted with EtOAc. The combined organic layers were washed with brine, dried over anhydrous Na₂SO₄, filtered, and concentrated under reduced pressure. The crude was purified by combi-flash chromatography using 40-43% EtOAc/Heptane to afford title compound 291 (0.72 g, 19%) as a brown gummy oil. LCMS calculated for C25H24N2O9S: 528.12; Found: 527.20 (M−1).

### Synthesis of methyl 3-((N-allyl-2-methoxy-6-(methoxymethoxy)phenyl)sulfonamido)-4-(benzyloxy)benzo[d]isoxazole-6-carboxylate (292)

To a stirred solution of compound 291 (1.20 g, 2.27 mmol) in DMF (20 mL) were added K₂CO₃ (1.57 g, 11.40 mmol) and 3-bromoprop-1-ene (1.37 g, 11.40 mmol). The reaction mixture was stirred at 50° C. for 2 h. After completion (monitored by TLC), the mixture was quenched with ice-cold water and was extracted with EtOAc. The combined organic layers were washed with brine, dried over anhydrous Na₂SO₄, filtered, and concentrated under reduced pressure. The crude was purified by combi-flash chromatography using 38-46% of EtOAc/Heptane to afford title compound 292 (0.75 g, 53.80%) as a brown solid. LCMS calculated for C28H28N2O9S: 568.15; Found: 569.30 (M+1).

### Synthesis of methyl 3-((N-allyl-2-hydroxy-6-methoxyphenyl)sulfonamido)-4-(benzyloxy)benzo[d]isoxazole-6-carboxylate (293)

To a stirred solution of compound 292 (0.90 g, 1.583 mmol) in DCM (85 mL) was added 1M BCl₃ in Toluene (2.53 mL, 2.533 mmol) at −78° C. The reaction mixture was stirred at −78° C. for 1 h. After completion (monitored by TLC), the mixture was quenched with ice-cold water and extracted with EtOAc. The combined organic layers were washed with brine, dried over anhydrous Na₂SO₄, filtered, and concentrated under reduced pressure. The crude was purified by combi-flash chromatography using 36-45% of EtOAc/Heptane to afford title compound 293 (0.63 g, 70.73%) as a brown gummy oil. LCMS calculated for C26H24N2O8S: 524.13; Found: 525.10 (M+1).

### Synthesis of methyl 3-((N-allyl-2-((5-bromopentyl)oxy)-6-methoxyphenyl)sulfonamido)-4-(benzyloxy)benzo[d]isoxazole-6-carboxylate (294)

To a stirred solution of compound 293 (0.63 g, 1.201 mmol) in DMF (16 mL) were added K₂CO₃ (0.74 g, 5.405 mmol) and 1,5-dibromopentane (0.49 mL, 3.603 mmol). The reaction mixture was stirred at 50° C. for 1 h. After completion (monitored by TLC), the mixture was quenched with ice-cold water and extracted with EtOAc. The combined organic layers were washed with brine, dried over anhydrous Na₂SO₄, and concentrated under reduced pressure. The crude was purified by combi-flash chromatography using 39-42% EtOAc/Heptane to afford title compound 294 (0.85 g, 89.34%) as a brown solid. LCMS calculated for C31H33BrN2O8S: 672.11; Found: 673.20 (M+1)

### Synthesis of methyl 3-((N-allyl-2-((5-bromopentyl)oxy)-6-methoxyphenyl)sulfonamido)-4-hydroxybenzo[d]isoxazole-6-carboxylate (295)

To a stirred solution of compound 294 (0.85 g, 1.262 mmol) in DCM (25 mL) was added 1M BCl₃ in toluene (8.80 mL, 8.833 mmol) at −78° C. The reaction mixture was stirred at −78° C. for 3 h. After completion (monitored by TLC), the mixture was quenched with ice-cold aqueous NaHCO₃ and extracted with EtOAc. The combined organic layers were washed with brine, dried over anhydrous Na₂SO₄, filtered, and concentrated under reduced pressure. The crude was purified by combi-flash chromatography using 36-45% EtOAc/Heptane to afford title compound 295 (0.63 g, 81.75%) as a brown gummy oil. LCMS calculated for C24H27BrN2O8S: 582.07; Found: 583.20 (M+1).

### Synthesis of methyl 16-allyl-14-methoxy-6,7,8,9-tetrahydro-5H,16H-4,10,18-trioxa-15-thia-16,17-diazabenzo[11,12]cyclotetradeca[1,2,3-cd]indene-2-carboxylate 15,15-dioxide (296)

To a stirred solution of compound 295 (0.63 g, 1.080 mmol) in DMSO (60 mL) were added K₂CO₃ (0.29 g, 2.160 mmol) and NaI (0.08 g, 0.539 mmol). The reaction mixture was stirred at 45° C. for 2 h. After completion (monitored by TLC), the mixture was quenched with ice-cold water and extracted with EtOAc. The combined organic layers were washed with brine, dried over anhydrous Na₂SO₄, filtered, and concentrated under reduced pressure. The crude was purified by combi-flash chromatography using 36-45% EtOAc/Heptane to afford title compound 296 (0.32 g, 56.34%) as a brown gummy oil. LCMS calculated for C24H26N2O8S: 502.14; Found: 503.30 (M+1).

### Synthesis of methyl 14-methoxy-6,7,8,9-tetrahydro-5H,16H-4,10,18-trioxa-15-thia-16,17-diazabenzo[11,12]cyclotetradeca[1,2,3-cd]indene-2-carboxylate 15,15-dioxide (297)

To a stirred solution of compound 296 (0.32 g, 0.636 mmol) in THF (32 mL) were added 1,3-dimethylbarbituric acid (0.33 g, 2.101 mmol) and Pd(PPh₃)₄ (0.33 g, 0.280 mmol). The reaction mixture stirred at 40° C. for 1 h. After completion (monitored by TLC), the mixture was quenched with ice-cold water and extracted with EtOAc. The combined organic layers were washed with brine, dried over anhydrous Na₂SO₄, filtered, and concentrated under reduced pressure. The crude was purified by combi-flash chromatography using 55-80% EtOAc/Heptane to afford title compound 297 (0.28 g, 84.65%) as a brown solid. LCMS calculated for C21H22N2O8S: 462.11; Found: 463.20 (M+1).

### Synthesis of 2-(hydroxymethyl)-14-methoxy-6,7,8,9-tetrahydro-5H,16H-4,10,18-trioxa-15-thia-16,17-diazabenzo[11,12]cyclotetradeca[1,2,3-cd]indene 15,15-dioxide (298)

To a stirred solution of compound 297 (0.28 g, 0.605 mmol) in THF (5 mL) was added 2M LiBH₄ in THF (0.91 mL, 1.816 mmol) at 0° C. The reaction mixture was stirred at 70° C. for 2 h. After completion (monitored by TLC), the mixture was quenched with ice-cold water and extracted with EtOAc. The combined organic layers were washed with brine, dried over anhydrous Na₂SO₄, filtered, and concentrated under reduced pressure. The crude was triturated with 9% DCM/pentane to afford title compound 298 (0.20 g, 65.20%) as a brown solid. LCMS calculated for C20H22N2O7S: 434.11; Found: 435.30 (M+1).

### Synthesis of tert-Butyl ((1-((14-methoxy-15,15-dioxido-6,7,8,9-tetrahydro-5H,16H-4,10,18-trioxa-15-thia-16,17-diazabenzo[11,12]cyclotetradeca[1,2,3-cd]inden-2-yl)methyl)-1H-pyrazol-4-yl)methyl)carbamate (299)

To a stirred solution of compound 298 (0.18 g, 0.414 mmol) in CH₃CN (8 mL) were added Cs₂CO₃ (0.53 g, 1.657 mmol) and compound 305 (0.22 g, 0.828 mmol). The reaction mixture was stirred at 90° C. for 4 h. After completion (monitored by TLC), the mixture was quenched with ice-cold water and extracted with EtOAc. The combined organic layers were washed with brine, dried over anhydrous Na₂SO₄, filtered, and concentrated under reduced pressure. The crude was purified by combi-flash chromatography using 10% MeOH/DCM to afford title compound 299 (80 mg, 18.80%) as a brown solid. LCMS calculated for C29H35N5O8S: 613.22; Found: 612.30 (M−1).

### Synthesis of 2-((4-(aminomethyl)-1H-pyrazol-1-yl)methyl)-14-methoxy-6,7,8,9-tetrahydro-5H,16H-4,10,18-trioxa-15-thia-16,17-diazabenzo[11,12]cyclotetradeca[1,2,3-cd]indene 15,15-dioxide (300)

To a stirred solution of compound 299 (0.08 g, 0.130 mmol) in DCM (2 mL) was added BF₃·Et₂O (0.37 mL, 0.195 mmol) at 0° C. The reaction mixture was stirred at room temperature for 1 h. After completion (monitored by TLC), the mixture was concentrated under reduced pressure. The crude was triturated with Et₂O to afford title compound 300 (0.07 g, 62.87%) as a brown gummy oil. LCMS calculated for C24H27N5O6S: 513.17; Found: 514.70 (M+1).

### Synthesis of methyl ((1-((14-methoxy-15,15-dioxido-6,7,8,9-tetrahydro-5H,16H-4,10,18-trioxa-15-thia-16,17-diazabenzo[11,12]cyclotetradeca[1,2,3-cd]inden-2-yl)methyl)-1H-pyrazol-4-yl)methyl)carbamate (Syn. Ex. 138)

To a stirred solution of compound 300 (0.07 g, 0.136 mmol) in DCM (3 mL) were added NEt₃ (0.06 mL, 0.408 mmol) and methyl chloroformate (0.01 mL, 0.149 mmol) at 0° C. The reaction mixture was stirred at 0° C. for 30 min. After completion (monitored by TLC), the mixture was quenched with ice-cold water and extracted with DCM. The combined organic layers were washed with brine, dried over anhydrous Na₂SO₄, filtered, and concentrated under reduced pressure. The crude was purified by prep-HPLC to afford title compound (Syn. Ex. 138; 9.00 mg, 11% yield) as an off-white solid. (See analytical data in Table 1).

### Synthesis of 1-methoxy-3-(methoxymethoxy)benzene (302)

To a stirred solution of 60% NaH in oil (8.37 g, 209.44 mmol) in DMF (200 mL) were added 3-methoxyphenol 301 (20 g, 161.11 mmol) and bromo(methoxy)methane (24.15 g, 193.33 mmol) at 0° C. The reaction mixture was stirred at room temperature for 2 h. After completion (monitored by TLC), the mixture was quenched with ice-cold water and extracted with EtOAc. The combined organic layers were washed with brine, dried over anhydrous Na₂SO₄, filtered, and concentrated under reduced pressure. The crude was purified by combi-flash chromatography using 12-15% of EtOAc/Heptane to afford title compound 302 (22 g, 78.53%) as a colourless oil. LCMS calculated for C9H12O3: 168.08; Found: 169.20 (M+1).

### Synthesis of lithium 2-methoxy-6-(methoxymethoxy)benzenesulfinate (303)

To a stirred solution of compound 302 (10 g, 59.50 mmol) in Hexane (200 mL) were added tetramethylethylenediamine (9.91 mL, 65.40 mmol) and 1.6M BuLi in Hexane (40.87 mL, 65.40 mmol) at 0° C. The reaction mixture was stirred at 0° C. for 30 min. The mixture was cooled at −70° C., purged with SO₂ for 20 min. and warmed at 10° C. After completion (monitored by TLC), the mixture was filtered, washed with Et₂O and concentrated under reduced pressure to afford title compound 303 (13.50 g, 86.20%) as a white solid; ¹H NMR (400 MHz, DMSO-d₆) δ ppm 7.10 (br t, J=8.11 Hz, 1H), 6.52-6.67 (m, 2H), 5.11-5.19 (m, 2H), 3.72 (s, 3H), 3.36 (s, 3H)

### Synthesis of 2-methoxy-6-(methoxymethoxy)benzenesulfonyl chloride (304)

To a stirred solution of compound 303 (7.50 g, 31 mmol) in Hexane (75 mL) was added SO₂Cl₂ (2.60 mL, 31 mmol) at 0° C. The reaction mixture was stirred at room temperature for 4 h. After completion (monitored by TLC), the mixture was quenched with ice-cold water and extracted with EtOAc. The combined organic layers were washed with brine, dried over anhydrous Na₂SO₄, filtered, and concentrated under reduced pressure to afford title compound 304 (3.60 g, 43%) as a brown solid; ¹H NMR (401 MHz, CHLOROFORM-d) δ ppm 7.47-7.57 (m, 1H), 6.87 (br d, J=8.31 Hz, 1H), 6.70 (br d, J=8.44 Hz, 1H), 5.33 (br s, 2H), 3.98 (br s, 3H), 3.55 (s, 3H).

### Synthesis of tert-Butyl ((1-(methylsulfonyl)-1H-pyrazol-4-yl)methyl)carbamate (305)

To a stirred solution of compound 19 (1 g, 5.070 mmol) in DMF (16 mL) were added sodium bis(trimethylsilyl)amide (1 mol/L) in THF (5.07 mL, 5.070 mmol) and MeSO₂Cl (0.44 mL, 5.577 mmol) at −40° C. The reaction mixture was stirred at room temperature for 3 h. After completion (monitored by TLC), the mixture was quenched with saturated aqueous NH₄Cl and extracted with EtOAc. The combined organic layers were washed with brine, dried over anhydrous Na₂SO₄, filtered and concentrated under reduced pressure. The crude was purified by combi-flash column chromatography using 35-60% EtOAc/Heptane to afford title compound 305 (0.60 g, 28.30%) as a white solid. LCMS calculated for C10H17N304S: 275.09; Found: 275.92 (M+1).

### Example 139

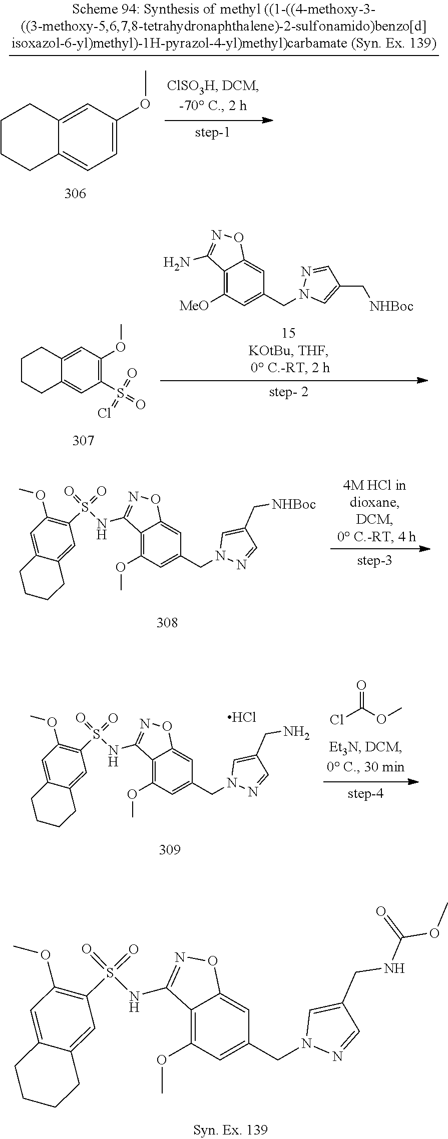

### Synthesis of 3-methoxy-5,6,7,8-tetrahydronaphthalene-2-sulfonyl chloride (307)

To a stirred solution of compound 306 (1 g, 6.164 mmol) in DCM (30 mL) was added ClSO₃H (1.30 mL, 18.492 mmol) at −70° C. The reaction mixture was stirred at −70° C. for 2 h. After completion (monitored by TLC), the mixture was quenched with saturated aqueous NaHCO₃ and extracted with DCM. The combined organic layers were dried over anhydrous Na₂SO₄ and concentrated under reduced pressure to afford title compound 307 (0.65 g, 40.44%) as an off-white solid; ¹H NMR (400 MHz, DMSO-d₆) δ ppm 7.38 (s, 1H), 6.68 (s, 1H), 3.71 (s, 3H), 2.59-2.73 (m, 4H), 1.70 (br t, J=2.87 Hz, 4H).

### Synthesis of tert-Butyl ((1-((4-methoxy-3-((3-methoxy-5,6,7,8-tetrahydronaphthalene)-2-sulfonamido)benzo[d]isoxazol-6-yl)methyl)-1H-pyrazol-4-yl)methyl)carbamate (308)

To a stirred solution of compound 15 (0.35 g, 0.959 mmol) in THF (20 mL) were added 1.6M KOtBu in THF (2.39 mL, 3.836 mmol) and compound 307 (0.50 g, 1.918 mmol) at 0° C. The reaction mixture was stirred at room temperature for 2 h. After completion (monitored by TLC), the mixture was quenched with saturated aqueous NaHCO₃ and extracted with EtOAc. The combined organic layers were dried over anhydrous Na₂SO₄ and concentrated under reduced pressure to afford title compound 308 (0.20 g, 6.93%). LCMS calculated for C29H35N5O7S: 597.23; Found: 596.30 (M−1).

### Synthesis of N-(6-((4-(aminomethyl)-1H-pyrazol-1-yl)methyl)-4-methoxybenzo[d]isoxazol-3-yl)-3-methoxy-5,6,7,8-tetrahydronaphthalene-2-sulfonamide hydrochloride (309)

To a solution of compound 308 (0.20 g, 0.334 mmol) in DCM (5 mL) was added 4M HCl in dioxane (4 mL) at 0° C. The reaction mixture was stirred at room temperature for 4 h. After completion (monitored by TLC), the mixture was concentrated under reduced pressure. The residue was washed with Et₂O and dried under reduced pressure to afford title compound 309 (0.15 g, 83.98%) as an off-white solid. LCMS calculated for C24H27N5O5S: 497.17; Found: 495.20 (M−2).

### Synthesis of methyl ((1-((4-methoxy-3-((3-methoxy-5,6,7,8-tetrahydronaphthalene)-2-sulfonamido)benzo[d]isoxazol-6-yl)methyl)-1H-pyrazol-4-yl)methyl)carbamate (Syn. Ex. 139)

To a stirred solution of compound 309 (0.15 g, 0.280 mmol) in DCM (5 mL) were added NEt₃ (0.11 mL, 0.842 mmol) methyl chloroformate (0.02 mL, 0.308 mmol) at 0° C. The reaction mixture was stirred at 0° C. for 30 min. After completion (monitored by TLC), the mixture was quenched with water and extracted with DCM. The combined organic layers were dried over anhydrous Na₂SO₄ and concentrated under reduced pressure. The crude was purified by prep. HPLC to afford methyl ((1-((4-methoxy-3-((3-methoxy-5,6,7,8-tetrahydronaphthalene)-2-sulfonamido)benzo[d]isoxazol-6-yl)methyl)-1H-pyrazol-4-yl)methyl)carbamate (Syn. Ex. 139; 40 mg, 25.25%) as an off-white solid. (See analytical data in Table 1).

### Example 140

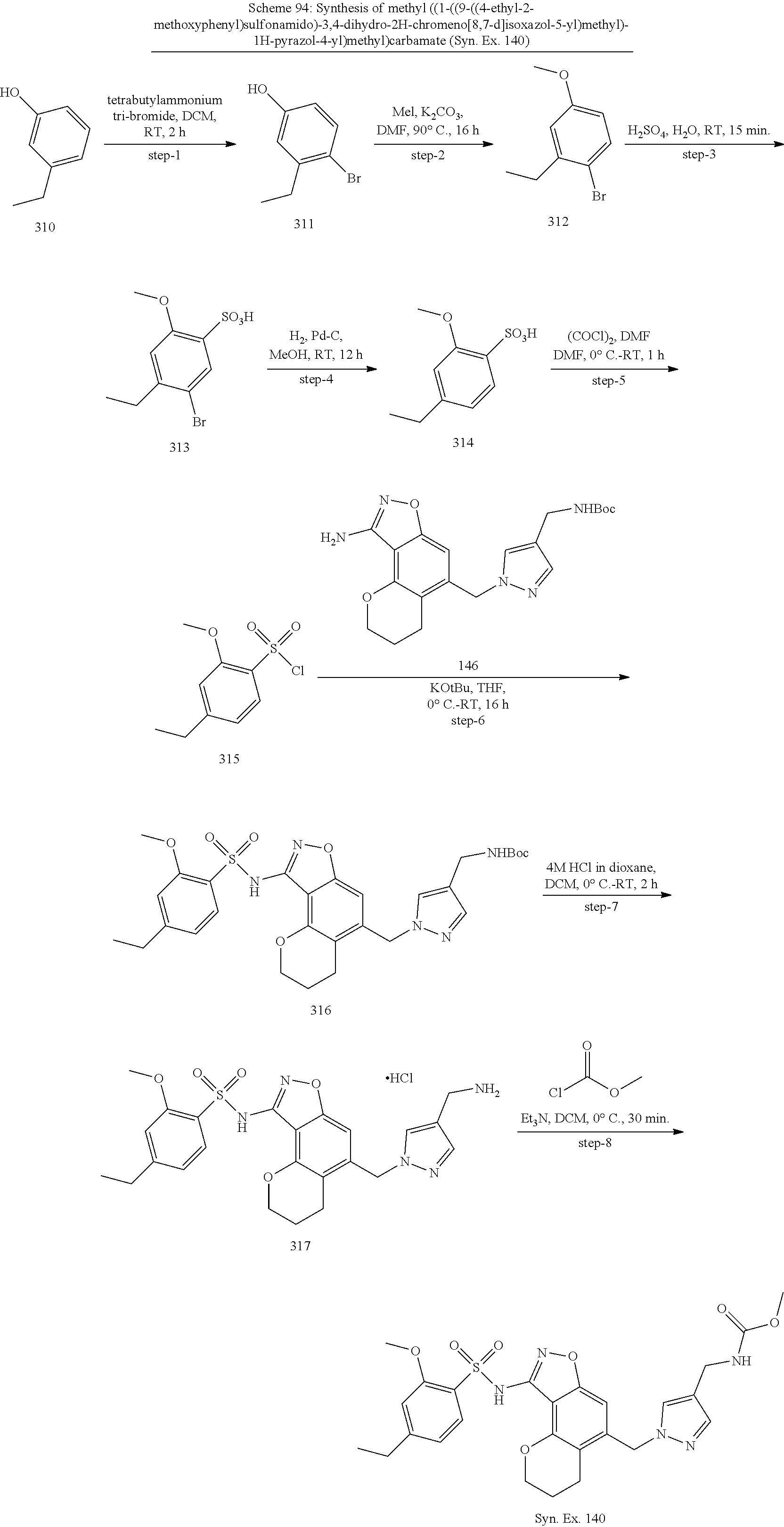

### Synthesis of 4-bromo-3-ethylphenol (311)

To a solution of 3-ethylphenol 310 (5 g, 40.930 mmol) in DCM (50 mL) was added tetrabutylammonium tribromide (19.73 g, 40.930 mmol) at 0° C. The reaction mixture was stirred at room temperature for 2 h. After completion (monitored by TLC), the mixture was concentrated under reduced pressure. The crude was washed with DCM, co-distilled with Et₂O and concentrated under reduced pressure to afford title compound 311 (5 g, 60.75%) as a liquid; ¹H NMR (400 MHz, DMSO-d₆) δ ppm 9.58 (s, 1H), 7.31 (br d, J=8.77 Hz, 1H), 6.73 (br d, J=1.75 Hz, 1H), 6.45-6.61 (m, 1H), 2.58 (q, J=7.45 Hz, 2H), 1.12 (br t, J=7.24 Hz, 3H)

### Synthesis of 1-bromo-2-ethyl-4-methoxybenzene (312)

To a solution of compound 311 (8 g, 39.789 mmol) in DMF (80 mL) were added K₂CO₃ (10.99 g, 79.578 mmol) and Mel (4.95 mL, 79.578 mmol). The reaction mixture was stirred at 90° C. for 16 h. After completion (monitored by TLC), the mixture was quenched with water and extracted with EtOAc. The combined organic layers were dried over anhydrous Na₂SO₄ and concentrated under reduced pressure to afford title compound 312 (8 g, 93.47%) as a liquid; ¹H NMR (401 MHz, DMSO-d₆) δ ppm 7.45 (d, J=8.68 Hz, 1H), 6.93 (d, J=3.06 Hz, 1H), 6.74 (dd, J=8.68, 3.06 Hz, 1H), 3.75 (s, 3H), 2.65 (q, J=7.50 Hz, 2H), 1.15 (t, J=7.52 Hz, 3H).

### Synthesis of 5-bromo-4-ethyl-2-methoxybenzenesulfonic acid (313)

A solution of compound 312 (3 g, 13.948 mmol) in H₂SO₄ (30 mL) was stirred at room temperature for 15 min. After completion (monitored by TLC), the mixture was quenched with ice-cold water (30 mL), concentrated under reduced pressure, filtered, washed with Heptane, and dried under reduced pressure to afford title compound 313 (4 g, 97.16%) as a white solid. LCMS calculated for C9H11BrO4S: 293.96; Found: 295 (M+1).

### Synthesis of 4-ethyl-2-methoxybenzenesulfonic acid (314)

To a solution of compound 313 (6 g, 20.329 mmol) in MeOH (150 mL) was added 10% Pd—C(1 g, 0.939 mmol). The reaction mixture was stirred at room temperature for 12 h under H₂ atmosphere (50 psi). After completion (monitored by TLC), the mixture was filtered through celite pad and concentrated under reduced pressure. The crude was purified by combi-flash chromatography using 0-5% MeOH/DCM to afford title compound 314 (4 g, 66.42%) as a brown colored liquid. LCMS calculated for C9H12O4S: 216.05; Found: 215.20 (M−1).

### Synthesis of 4-ethyl-2-methoxybenzenesulfonyl chloride (315)

To a stirred solution compound 314 (2 g, 9.248 mmol) in DCM (20 mL) and DMF (0.07 mL) was added oxalyl chloride (8.09 mL, 92.486 mmol) at 0° C. The reaction was stirred at room temperature for 1 h. The reaction was monitored by TLC. After completion (monitored by TLC), the mixture was quenched with ice-cold water and extracted with EtOAc. The combined organic layers were washed with brine, dried over anhydrous Na₂SO₄, filtered, and concentrated under reduced pressure to afford title compound 315 (0.25 g, 11.51%) as a brown liquid; ¹H NMR (400 MHz, DMSO-d₆) δ ppm 7.56 (br d, J=7.88 Hz, 1 H), 6.80 (s, 1H), 6.69 (br d, J=7.46 Hz, 1H), 3.77 (br s, 3H), 2.54-2.62 (m, 2H), 1.17 (br t, J=7.46 Hz, 3H)

### Synthesis of tert-Butyl ((1-((9-((4-ethyl-2-methoxyphenyl)sulfonamido)-3,4-dihydro-2H-chromeno[8,7-d]isoxazol-5-yl)methyl)-1H-pyrazol-4-yl)methyl)carbamate (316)

To a stirred solution compound 146 (0.30 g, 0.751 mmol) in dry THF (10 mL) was added 1.6M KOtBu in THF (1.40 mL, 2.253 mmol) at 0° C. The reaction was stirred at 0° C. for 10 min. followed by addition of compound 315 (0.35 g, 1.502 mmol). The reaction mixture was stirred at room temperature for 16 h. After completion (monitored by TLC), the mixture was quenched with ice-cold water and extracted with EtOAc. The aqueous layer was acidified with 1N HCl to pH˜6 and extracted with EtOAc. The combined organic layers were washed with brine, dried over anhydrous Na₂SO₄, filtered, and concentrated under reduced pressure to afford title compound 316 (0.40 g, 58.80%) as a brown solid. LCMS calculated for C29H35N5O7S: 597.23; Found: 596.30 (M−1).

### Synthesis of N-(5-((4-(aminomethyl)-1H-pyrazol-1-yl)methyl)-3,4-dihydro-2H-chromeno[8,7-d]isoxazol-9-yl)-4-ethyl-2-methoxybenzenesulfonamide hydrochloride (317)

To a solution of compound 316 (0.40 g, 0.669 mmol) in DCM (10 mL) was added 4M HCl in dioxane (0.83 mL, 3.346 mmol) at 0° C. The reaction mixture was stirred at room temperature for 2 h. After completion (monitored by TLC), the mixture was concentrated under reduced pressure. The residue was washed with DCM, co-distilled with Et₂O and concentrated under reduced pressure to afford title compound 317 (0.20 g, 32.43%) as an off-white solid. LCMS calculated for C24H27N5O5S: 497.17; Found: 498.60 (M+1).

### Synthesis of methyl ((1-((9-((4-ethyl-2-methoxyphenyl)sulfonamido)-3,4-dihydro-2H-chromeno[8,7-d]isoxazol-5-yl)methyl)-1H-pyrazol-4-yl)methyl)carbamate (Syn. Ex. 140)

To a solution of compound 317 (0.20 g, 0.374 mmol) in DCM (10 mL) were added NEt₃ (0.15 mL, 1.123 mmol) and methyl chloroformate (0.03 mL, 0.486 mmol) in DCM at 0° C. The reaction mixture was stirred at 0° C. for 30 min. After completion (monitored by TLC), the mixture was quenched with water and extracted with DCM. The combined organic layers were washed with brine, dried over anhydrous Na₂SO₄, filtered, and concentrated under reduced pressure. The crude was purified using prep. HPLC to afford title compound (Syn. Ex. 140; 14 mg, 6.52%) as an off-white solid.

### Example 141

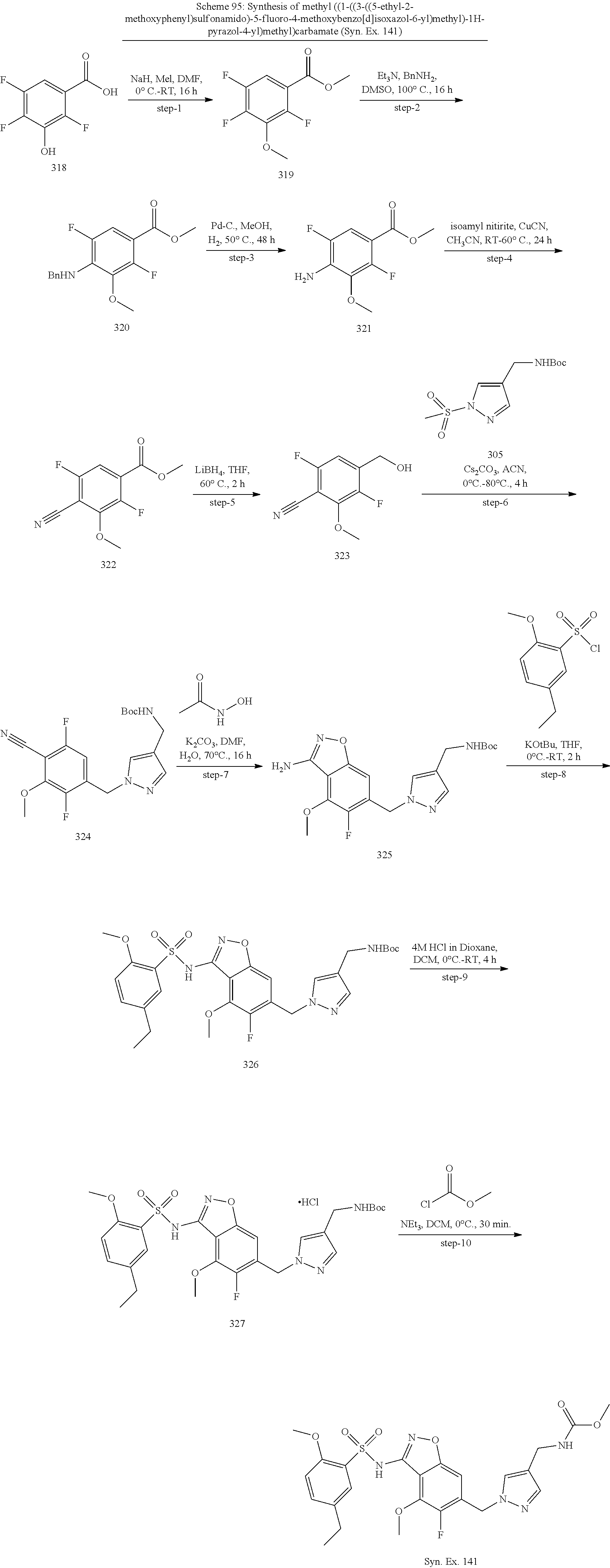

### Synthesis of methyl 2,4,5-trifluoro-3-methoxybenzoate (319)

To a stirred solution of compound 318 (5 g, 26 mmol) in DMF (50 mL) was added NaH (3 g, 110 mmol) at 0° C. The reaction mixture was stirred at 0° C. for 20 min followed by addition of Mel (2 g, 14 mmol). The reaction mixture was stirred at room temperature 12 h. After completion (monitored by TLC), the mixture was acidified with citric acid and extracted with EtOAc. The combined organic layers were dried over anhydrous Na₂SO₄ and concentrated under reduced pressure. The crude was purified by combi-flash column chromatography using 10-15% EtOAc/Hexane and concentrated under reduced pressure to afford title compound 319 (3.50 g, 61.07%) as colourless liquid; ¹H NMR (400 MHz, DMSO-d₆) δ 7.56-7.68 (m, 1H), 4.01 (s, 3H), 3.86 (s, 3H)

### Synthesis of methyl 4-(benzylamino)-2,5-difluoro-3-methoxybenzoate (320)

To a stirred solution of compound 319 (18 g, 81.766 mmol) in DMSO (180 mL) was added BnNH₂ (14 mL, 122.65 mmol) at 0° C. The reaction mixture was stirred at 0° C. for 20 min. followed by addition of Et₃N (34.40 mL, 245.30 mmol). The reaction mixture was stirred at 100° C. for 12 h. After completion (monitored by TLC), the mixture was quenched with ice-cold water and extracted with EtOAc. The combined organic layers were dried over anhydrous Na₂SO₄ and concentrated under reduced pressure. The crude was purified by combi-flash column chromatography using 10-15% EtOAc in Hexane to afford title compound 320 (12 g, 41.50%) as a colourless liquid. LCMS calculated for C16H15F2NO3: 307.10; Found: 308.20 (M+1).

### Synthesis of methyl 4-amino-2,5-difluoro-3-methoxybenzoate (321)

To a stirred solution of compound 320 (2.40 g, 7.80 mmol) in MeOH (24 mL) was added 10% Pd—C(0.50 g) at room temperature. The reaction mixture was stirred at 50° C. for 48 h under H₂ atmosphere (50 psi). After completion (monitored by TLC), the mixture was filtered through celite bed, washed with MeOH (10 mL) and concentrated under reduced pressure to afford title compound 321 (1.40 g, 75%) as an off-white solid. LCMS calculated for C9H9F2NO3: 217.06; Found: 218.20 (M+1).

### Synthesis of methyl 4-cyano-2,5-difluoro-3-methoxybenzoate (322)

To a stirred solution of compound 321 (6 g, 27.628 mmol) in ACN (60 mL) was added CuCN (7.49 g, 82.884 mmol) at room temperature. The reaction mixture was stirred at 60° C. for 24 h followed by addition of isoamyl nitrite (10.01 g, 82.884 mmol). After completion (monitored by TLC), the mixture was filtered through celite bed, washed with MeOH and concentrated under reduced pressure. The crude was purified by combi-flash chromatography using 20% EtOAc/Hexane to afford title compound 322 (3 g, 47.79%) as a brown solid; ¹H NMR (400 MHz, DMSO-d₆) δ 7.52-7.63 (m, 1H), 4.12-4.18 (m, 3H), 3.86-3.92 (m, 3H)

### Synthesis of 3,6-difluoro-4-(hydroxymethyl)-2-methoxybenzonitrile (323)

To a stirred solution of compound 322 (3 g, 13 mmol) in THF (30 mL) was added LiBH₄ (0.58 g, 26 mmol) at 0° C. The reaction mixture was stirred at 60° C. for 2 h. After completion (monitored by TLC), the mixture was quenched with ice cold water, extracted with EtOAc and concentrated under reduced pressure. The crude was purified by combi-flash chromatography using 50% EtOAc/Heptane to afford title compound 323 (2.10 g, 79.84%) as an off-white solid; ¹H NMR (400 MHz, CHLOROFORM-d) δ 7.04 (dd, J=8.63, 4.75 Hz, 1 H), 4.81 (s, 2H), 4.15 (s, 3H)

### Synthesis of tert-Butyl ((1-(4-cyano-2,5-difluoro-3-methoxybenzyl)-1H-pyrazol-4-yl)methyl)carbamate (324)

To a stirred solution of compound 305 (10 mg, 0.05 mmol) in ACN (4 mL) were added Cs₂CO₃ (0.04 g, 0.15 mmol) and compound 323 (0.01 g, 0.05 mmol) at 0° C. The reaction mixture was stirred at 80° C. for 4 h. After completion (monitored by TLC), the mixture was cooled at room temperature, filtered and concentrated under reduced pressure. The crude was purified by combi-flash chromatography using 50% EtOAc/Hexane to afford title compound 324 (8 mg, 42.32%) as an off-white solid. LCMS calculated for C18H20F2N4O3: 378.15; Found: 379 (M+1).

### Synthesis of 6-((4-(aminomethyl)-1H-pyrazol-1-yl)methyl)-5-fluoro-4-methoxybenzo[d]isoxazol-3-amine (325)

To a stirred solution of compound 324 (0.05 g, 0.1321 mmol) in DMF (4 mL) were added K₂CO₃ (0.05 g, 0.396 mmol), acetohydroxamic acid (0.02 g, 0.396 mmol) and water (2 mL). The reaction mixture was stirred at 70° C. for 16 h. After completion (monitored by TLC), the mixture was diluted with water and extracted with EtOAc. The combined organic layers were dried over anhydrous Na₂SO₄, filtered, and concentrated under reduced pressure. The crude was purified by combi-flash chromatography using 70-80% EtOAc/Hexane to afford title compound 325 (30 mg, 28.54%) as a pale-yellow solid. LCMS calculated for C18H22FN5O4: 391.17; Found: 392 (M+1).

### Synthesis of tert-Butyl ((1-((3-((5-ethyl-2-methoxyphenyl)sulfonamido)-5-fluoro-4-methoxybenzo[d]isoxazol-6-yl)methyl)-1H-pyrazol-4-yl)methyl)carbamate (326)

To a stirred solution of compound 325 (0.03 g, 0.0766 mmol) in THF (5 mL) were added 1.6M KOtBu in THF (0.09 mL, 0.153 mmol) and 5-ethyl-2-methoxybenzenesulfonyl chloride (0.02 g, 0.076 mmol) at 0° C. The reaction mixture was stirred at room temperature for 2 h. After completion (monitored by TLC), the mixture was quenched with ice cold water and extracted with DCM. The combined organic layers were dried over anhydrous Na₂SO₄ and concentrated under reduced pressure to afford title compound 326 (0.03 g, 25.10%) as an off-white solid. LCMS calculated for C27H32FN5O7S: 589.20; Found: 590 (M+1).

### Synthesis of N-(6-((4-(aminomethyl)-1H-pyrazol-1-yl)methyl)-5-fluoro-4-methoxybenzo[d]isoxazol-3-yl)-5-ethyl-2-methoxybenzenesulfonamide hydrochloride (327)

To a solution of compound 326 (30 mg, 0.050 mmol) in DCM (4 mL) was added 4M HCl in dioxane (0.05 mL, 0.203 mmol) at 0° C. The reaction mixture was stirred at room temperature for 4 h. After completion (monitored by TLC), the mixture was concentrated under reduced pressure. The residue was washed with Et₂O and dried under reduced pressure to afford title compound 327 (25 mg, 31.84% yield) as an off-white solid. LCMS calculated for C22H24FN5O5S: 489.15; Found: 488.20 (M−1).

### Synthesis of methyl ((1-((3-((5-ethyl-2-methoxyphenyl)sulfonamido)-5-fluoro-4-methoxybenzo[d]isoxazol-6-yl)methyl)-1H-pyrazol-4-yl)methyl)carbamate (Syn. Ex. 141)

To a solution of compound 327 (25 mg, 0.047 mmol) in DCM (2 mL) were added NEt₃ (0.01 mL, 0.142 mmol) and methyl chloroformate (0.003 mL, 0.047 mmol) at 0° C. The reaction mixture was stirred at 0° C. for 30 min. After completion (monitored by TLC), the mixture was quenched with water and extracted with DCM. The combined organic layers were dried over anhydrous Na₂SO₄ and concentrated under reduced pressure. The crude was purified by prep. HPLC to afford methyl ((1-((3-((5-ethyl-2-methoxyphenyl)sulfonamido)-5-fluoro-4-methoxybenzo[d]isoxazol-6-yl)methyl)-1H-pyrazol-4-yl)methyl)carbamate (Syn. Ex. 141; 8 mg, 30.18%) as an off-white solid. (See analytical data in Table 1).

### Example 142

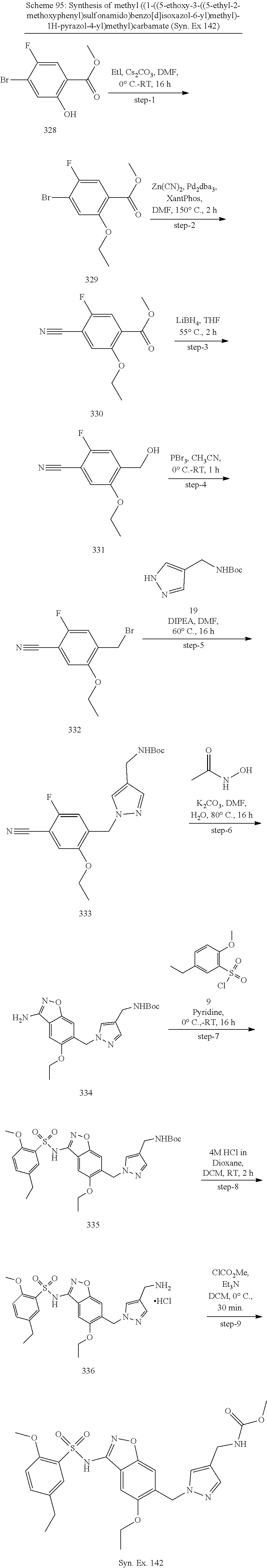

### Synthesis of methyl 4-bromo-2-ethoxy-5-fluorobenzoate (329)

To a stirred solution of compound 328 (1 g, 4 mmol) in DMF (12 mL) were added Cs₂CO₃ (2 g, 6.0 mmol) and iodoethane (0.95 g, 6.0 mmol) at 0° C. The reaction mixture was stirred at room temperature for 16 h. After completion (monitored by TLC), the mixture was quenched with ice-cold water and extracted with EtOAc. The combined organic layers were dried over anhydrous Na₂SO₄, filtered and concentrated under reduced pressure to afford title compound 329 (1.1 g, 98%) as a white solid. TLC: 10% EtOAc/Heptane (R_{f}: 0.4). LCMS calculated for C₁₀H₁₀BrFO₃: 275.98; Found: 277.0 (M+1).

### Synthesis of methyl 4-cyano-2-ethoxy-5-fluorobenzoate (330)

To a stirred solution of compound 329 (6.5 g, 23 mmol) in DMF (80 mL) was added Zn(CN)₂ (4.2 g, 35 mmol). The reaction mixture was degassed with N₂ for 5 min. followed by addition of XantPhos (1.4 g, 2.3 mmol) and Pd₂(dba)₃ (1.1 g, 1.2 mmol) and again degassed for 2 min. The reaction mixture was stirred at 150° C. for 2 h. After completion (monitored by TLC), the mixture was quenched with ice-cold water and extracted with EtOAc. The combined organic layers were dried over anhydrous Na₂SO₄, filtered and concentrated under reduced pressure. The crude was purified by combi-flash chromatography using 19-26% EtOAc/Heptane to afford title compound 330 (4.4 g, 83%) as a brown solid. TLC: 25% EtOAc/Heptane (R_{f}: 0.4); ¹H NMR (400 MHz, DMSO-d₆) δ ppm 7.66-7.78 (m, 2H) 4.13 (q, J=6.91 Hz, 2H) 3.83 (s, 3H) 1.31 (t, J=7.05 Hz, 3H)

### Synthesis of 5-ethoxy-2-fluoro-4-(hydroxymethyl)benzonitrile (331)

To a stirred solution of methyl 4-cyano-2-ethoxy-5-fluorobenzoate 330 (4.3 g, 19 mmol) in THF (50 mL) was added 2M LiBH₄ in THF (29 mL, 58 mmol) at room temperature. The reaction mixture was stirred at 55° C. for 2 h. After completion (monitored by TLC), the mixture was added to ice-cold aqueous NH₄Cl and extracted with EtOAc. The combined organic layers were washed with brine, dried over anhydrous Na₂SO₄, filtered and concentrated under reduced pressure. The crude was triturated with DCM/Heptane (9:1) to afford title compound 331 (3.4 g, 86%) as a white solid. TLC: 40% EtOAc/Heptane, (R_{f}: 0.45); ¹H NMR (401 MHz, DMSO-d₆) δ ppm 7.45 (d, J=5.14 Hz, 1H) 7.39 (d, J=9.78 Hz, 1H) 4.52 (d, J=5.38 Hz, 2H) 4.08 (q, J=6.93 Hz, 2H) 1.32 (t, J=6.85 Hz, 3H)

### Synthesis of 4-(bromomethyl)-5-ethoxy-2-fluorobenzonitrile (332)

To a stirred solution of compound 331 (3.4 g, 17 mmol) in ACN (40 mL) was added PBr₃ (3.3 mL, 35 mmol) at 0° C. Then reaction mixture was warm to room temperature and stirred for 1 h. After completion (monitored by TLC), the mixture was added to saturated NaHCO₃ and extracted with EtOAc. The combined organic layers were dried over anhydrous Na₂SO₄, filtered and concentrated under reduced pressure. The crude was purified by combi-flash chromatography using 15-20% EtOAc/Heptane to afford title compound 332 (2.45 g, 54%) as a white solid. TLC: 10% EtOAc/Heptane, (R_{f}: 0.4); ¹H NMR (400 MHz, DMSO-d₆) δ ppm 7.65 (d, J=9.54 Hz, 1H) 7.58 (d, J=5.39 Hz, 1H) 4.62 (s, 2H) 4.16 (q, J=7.05 Hz, 2H) 1.36 (t, J=6.84 Hz, 3H).

### Synthesis of tert-butyl ((1-(4-cyano-2-ethoxy-5-fluorobenzyl)-1H-pyrazol-4-yl)methyl)carbamate (333)

To a stirred solution of compound 332 (2 g, 7.7 mmol) in DMF (20 mL) were added DIPEA (4.1 mL, 23 mmol) and compound 19 (1.8 g, 9.3 mmol) at 0° C. The reaction mixture was stirred at 60° C. for 16 h. After completion (monitored by TLC), the mixture was quenched with ice-cold water and extracted with EtOAc. The combined organic layers were washed with brine, dried over anhydrous Na₂SO₄, filtered and concentrated under reduced pressure. The crude was purified by combi-flash chromatography using 40-51% EtOAc/Heptane to afford title compound 333 (1.25 g, 39%) as a white solid. TLC: 50% EtOAc/Heptane, (R_{f}: 0.4). LCMS calculated for C₁₉H₂₃FN₄O₃: 374.18; Found: 375.2 (M+1).

### Synthesis of tert-butyl ((1-((3-amino-5-ethoxybenzo[d]isoxazol-6-yl)methyl)-1H-pyrazol-4-yl)methyl)carbamate (334)

To a stirred solution of compound 333 (1.25 g, 3.34 mmol) in DMF (36 mL) and water (9 mL) were added acetohydroxamic acid (0.75 g, 10.0 mmol) and K₂CO₃ (2.3 g, 16.7 mmol). The reaction mixture was stirred at 80° C. for 16 h. After completion (monitored by TLC), the mixture was diluted with water and extracted with EtOAc. The combined organic layers were dried over anhydrous Na₂SO₄ and concentrated under reduced pressure. The crude was purified by combi-flash chromatography using 50-62% EtOAc/Heptane to afford title compound 334 (0.32 g, 13.1%) as a brown solid. LCMS calculated for C₁₉H₂₅N₅O₄: 387.19; Found: 388.3 (M+1).

### Synthesis of tert-butyl ((1-((5-ethoxy-3-((5-ethyl-2-methoxyphenyl)sulfonamido)benzo[d]isoxazol-6-yl)methyl)-1H-pyrazol-4-yl)methyl)carbamate (335)

To a stirred solution of compound 334 (0.4 g, 1.033 mmol) in pyridine (3 mL) was added 5-ethyl-2-methoxybenzenesulfonyl chloride (0.29 g, 1.239 mmol) at room temperature. The reaction mixture was stirred at 95° C. for 16 h. After completion (monitored by TLC), the mixture was quenched with water and extracted with EtOAc. The combined organic layers were dried over anhydrous Na₂SO₄ and concentrated under reduced pressure. The crude was purified by combi-flash chromatography using 50-60% EtOAc/Heptane to afford title compound 335 (75 mg, 1.66%) as a brown solid. LCMS calculated for C28H₃₅N₅O₇S: 585.23; Found: 586.4 (M+1).

### Synthesis of N-(6-((4-(aminomethyl)-1H-pyrazol-1-yl)methyl)-5-ethoxybenzo[d]isoxazol-3-yl)-5-ethyl-2-methoxybenzenesulfonamide hydrochloride (336)

To a stirred solution of compound 335 (115 mg, 0.1963 mmol) in DCM (3 mL) was added 4M HCl in 1,4-Dioxane (0.24 mL, 0.981 mmol). The reaction mixture was stirred at room temperature for 2 h. After completion (monitored by TLC), the mixture was concentrated under reduced pressure. The crude was triturated with Et₂O to afford title compound 336 (95 mg, 84.48%) as a brown solid. LCMS calculated for C₂₃H₂₇N₅O₅S: 485.17; Found: 484.0 (M−1).

### Synthesis of methyl ((1-((5-ethoxy-3-((5-ethyl-2-methoxyphenyl)sulfonamido)benzo[d]isoxazol-6-yl)methyl)-1H-pyrazol-4-yl)methyl)carbamate (Syn. Ex. 142)

To a stirred solution compound 336 (80 mg, 0.164 mmol) in DCM (3 mL) was added Et₃N (0.073 mL, 0.4942 mmol) and methyl chloroformate (0.012, 0.163 mmol) at 0° C. The reaction mixture was stirred at 0° C. for 30 min. After completion (monitored by TLC), the mixture was quenched with ice-cold water and extracted with DCM. The combined organic layers were dried over anhydrous Na₂SO₄, filtered and concentrated under reduced pressure. The crude was purified by prep. HPLC to afford methyl ((1-((5-ethoxy-3-((5-ethyl-2-methoxyphenyl)sulfonamido)benzo[d]isoxazol-6-yl)methyl)-1H-pyrazol-4-yl)methyl)carbamate (Syn. Ex. 142; 20 mg, 21.58%) as an off-white solid.

### Example 143

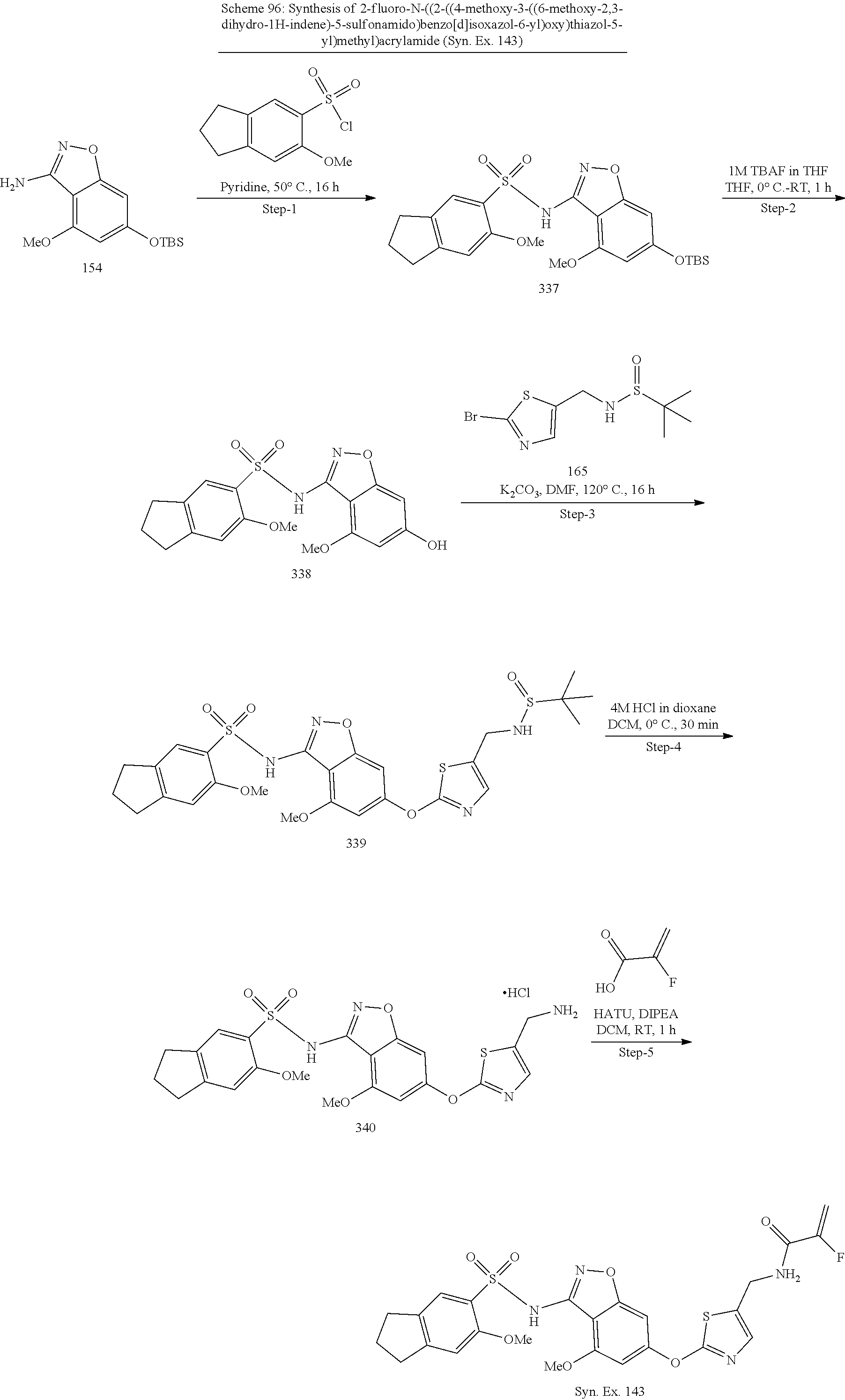

### Synthesis of N-(6-((tert-butyldimethylsilyl)oxy)-4-methoxybenzo[d]isoxazol-3-yl)-6-methoxy-2,3-dihydro-1H-indene-5-sulfonamide (337)

To a stirred solution of compound 154 (1 g, 3.4 mmol) in Pyridine (10 mL) was added 6-methoxy-2,3-dihydro-1H-indene-5-sulfonyl chloride (2.2 g, 8.9 mmol). The reaction mixture was stirred at 50° C. for 16 h. After completion (monitored by TLC), the mixture was quenched with ice-cold water and extracted with EtOAc. The combined organic layers were washed with brine, dried over anhydrous Na₂SO₄, filtered and concentrated under reduced pressure. The crude was purified by combi-flash chromatography to afford title compound 337 (0.48 g, 25%) as a brown solid. LCMS calculated for C₂₄H₃₂N2O₆SSi: 504.18; Found: 505.0 (M+1).

### Synthesis of N-(6-hydroxy-4-methoxybenzo[d]isoxazol-3-yl)-6-methoxy-2,3-dihydro-1H-indene-5-sulfonamide (338)

To a stirred solution of compound 337 (0.48 g, 0.951 mmol) in THF (8 mL) was added 1M TBAF in THF (1.1 mL, 1.141 mmol) at 0° C. The reaction mixture was stirred at room temperature for 1 h. After completion (monitored by TLC), the mixture was quenched with ice-cold water and extracted with EtOAc. The combined organic layers were washed with brine, dried over anhydrous Na₂SO₄, filtered and concentrated under reduced pressure to afford title compound 138 (0.25 g, 63.04%) as a brown solid. LCMS calculated for C₁₈H₁₈N₂O₆S: 390.09; Found: 391.0 (M+1).

### Synthesis of N-(6-((5-(((tert-butylsulfinyl)amino)methyl)thiazol-2-yl)oxy)-4-methoxybenzo[d]isoxazol-3-yl)-6-methoxy-2,3-dihydro-1H-indene-5-sulfonamide (339)

To a stirred solution of compound 338 (0.5 g, 1.281 mmol) in DMF (5 mL) were added K₂CO₃ (0.53 g, 3.842 mmol) and compound 165 (0.45 g, 1.537 mmol). The reaction mixture was stirred at 120° C. for 16 h. After completion (monitored by TLC), the mixture was quenched with ice-cold water and extracted with EtOAc. The combined organic layers were washed with brine, dried over anhydrous Na₂SO₄, filtered and concentrated under reduced pressure. The crude was purified by combi-flash chromatography to afford title compound 339 (0.13 g, 9.53%) as an off white solid. LCMS calculated for C₂₆H₃₀N₄O₇S₃: 606.13; Found: 607.0 (M+1).

### Synthesis of N-(6-((5-(aminomethyl)thiazol-2-yl)oxy)-4-methoxybenzo[d]isoxazol-3-yl)-6-methoxy-2,3-dihydro-1H-indene-5-sulfonamide hydrochloride (340)

To a stirred solution of compound 339 (0.13 g, 0.214 mmol) in DCM (3 mL) was added 4M HCl in 1,4-Dioxane (0.11 mL, 0.4285 mmol) at 0° C. The reaction mixture was stirred at 0° C. for 30 min. After completion (monitored by TLC), the mixture was concentrated under reduced pressure. The residue was washed with Et₂O and concentrated under vacuum to afford title compound 340 (0.15 g, 65.46%) as a brown gummy. LCMS calculated for C22H22N4O6S2: 502.10; Found: 504.27 (M+2).

### Synthesis of 2-fluoro-N-((2-((4-methoxy-3-((6-methoxy-2,3-dihydro-1H-indene)-5-sulfonamido)benzo[d]isoxazol-6-yl)oxy)thiazol-5-yl)methyl)acrylamide (Syn. Ex. 143)

To a stirred solution of compound 340 (0.14 g, 0.278 mmol) in DCM (2 mL) were added DIPEA (0.1 g, 0.835 mmol), HATU (0.16 g, 0.417 mmol) and 2-fluoroacrylic acid (0.03 g, 3343 mmol) at rt. The reaction mixture was stirred at room temperature for 1 h. After completion (monitored by TLC), the mixture was quenched with ice-cold water and extracted with DCM. The combined organic layers were dried over anhydrous Na₂SO₄, filtered and concentrated under reduced pressure. The crude was purified by prep. HPLC to afford 2-fluoro-N-((2-((4-methoxy-3-((6-methoxy-2,3-dihydro-1H-indene)-5-sulfonamido)benzo[d]isoxazol-6-yl)oxy)thiazol-5-yl)methyl)acrylamide Syn. Ex. 143 (0.013 g, 8.09%) as an off white solid.

### Example 144

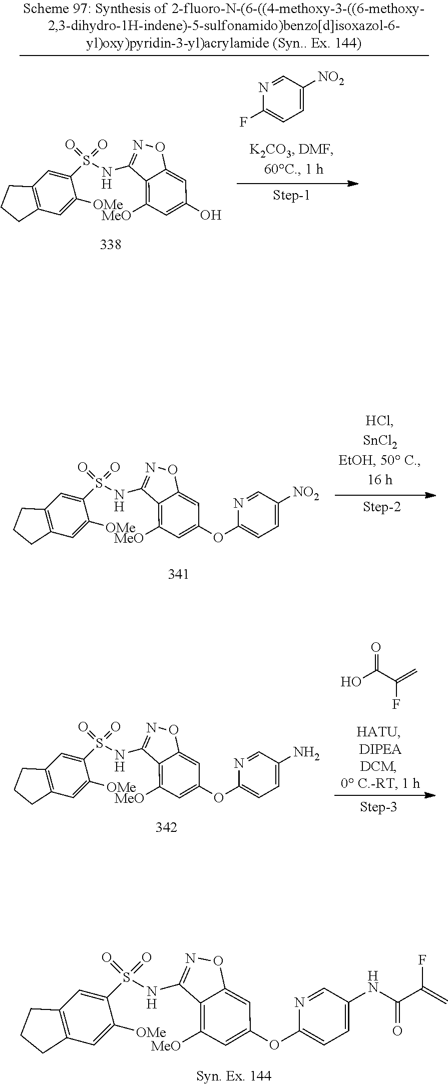

### Synthesis of 6-methoxy-N-(4-methoxy-6-((5-nitropyridin-2-yl)oxy)benzo[d]isoxazol-3-yl)-2,3-dihydro-1H-indene-5-sulfonamide (341)

To a stirred solution of compound 338 (0.22 g, 0.563 mmol) in DMF (3 mL) were added K₂CO₃ (0.15 g, 1.127 mmol) and 2-fluoro-5-nitropyridine (0.096 g, 0.676 mmol). The reaction mixture was stirred at 60° C. for 1 h. After completion (monitored by TLC), the mixture was quenched with ice-cold water, acidified with 1N HCl and extracted with EtOAc. The combined organic layers were washed with brine, dried over anhydrous Na₂SO₄, filtered and concentrated under reduced pressure. The crude was purified by combi-flash chromatography to afford title compound 341 (0.15 g, 38.26%) as a brown solid. LCMS calculated for C₂₃H₂₀N₄O₈S: 512.10; Found: 512.9 (M+1).

### Synthesis of N-(6-((5-aminopyridin-2-yl)oxy)-4-methoxybenzo[d]isoxazol-3-yl)-6-methoxy-2,3-dihydro-1H-indene-5-sulfonamide (342)

To a stirred solution of compound 341 (0.18 g, 0.351 mmol) in EtOH (3 mL) were added HCl (0.026 mL, 1.054 mmol) and Tin(II) chloride (0.206 g, 1.054 mmol). The reaction mixture was stirred at 50° C. for 16 h. After completion (monitored by TLC), the mixture was quenched with saturated NaHCO₃ solution and extracted with EtOAc (3×30 mL). The combined organic layers were dried over anhydrous Na₂SO₄, filtered and concentrated under reduced pressure. The residue was washed with Et₂O to afford title compound 342 (0.15 g, 69.3%) as a brown solid. LCMS calculated for C23H₂₂N4O₆S: 482.13; Found: 483.1 (M+1).

### Synthesis of 2-fluoro-N-(6-((4-methoxy-3-((6-methoxy-2,3-dihydro-1H-indene)-5-sulfonamido)benzo[d]isoxazol-6-yl)oxy)pyridin-3-yl)acrylamide (Syn. Ex. 144)

To a stirred solution of compound 342 (0.06 g, 0.124 mmol) in DCM (2 mL) were added DIPEA (0.064 mL, 0.373 mmol), HATU (0.073 g, 0.186 mmol) and 2-fluoroacrylic acid (0.013 g, 0.149 mmol) at 0° C. The reaction mixture was stirred at room temperature for 1 h. After completion (monitored by TLC), the mixture was quenched with ice-cold water and extracted with DCM (3×20 mL). The combined organic layers were dried over anhydrous Na₂SO₄, filtered and concentrated under reduced pressure. The crude was purified by prep. HPLC to afford 2-fluoro-N-(6-((4-methoxy-3-((6-methoxy-2,3-dihydro-1H-indene)-5-sulfonamido)benzo[d]isoxazol-6-yl)oxy)pyridin-3-yl)acrylamide Syn. Ex. 144 (9.00 mg, 13%) as an off white solid.

### Example 145

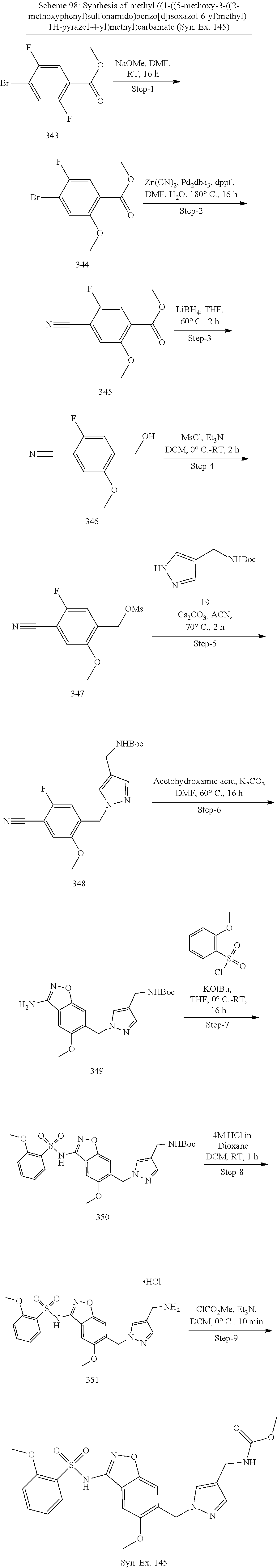

### Synthesis of methyl 4-bromo-5-fluoro-2-methoxybenzoate (344)

To a stirred solution of compound 343 (10 g, 39.837 mmol) in DMF (100 mL) was added NaOMe (2.83 g, 49.797 mmol). The reaction mixture was stirred at room temperature for 16 h. After completion (monitored by TLC), the mixture was quenched with cold water and extracted with EtOAc (3×150 mL). The combined organic layers were dried over anhydrous Na₂SO₄ and concentrated under reduced pressure. The crude was purified by combi-flash chromatography to afford title compound 344 (10.2 g, 89.1%) as a white solid. LCMS calculated for C9H8BrFO3: 261.96; Found: 264.6 (M+2).

### Synthesis of methyl 4-cyano-5-fluoro-2-methoxybenzoate (345)

To a stirred solution of compound 344 (8.5 g, 32 mmol) in DMF (100 mL) was added Zn(CN)₂ (5.7 g, 48 mmol). The reaction mixture was degassed with N₂ for 5 min. followed by addition of XantPhos (1.9 g, 3.2 mmol) and Pd₂(dba)₃ (1.5 g, 1.6 mmol) and again degassed for 2 min. The reaction mixture was stirred at 150° C. for 2 h. After completion (monitored by TLC), the mixture was quenched with ice-cold water and extracted with EtOAc. The combined organic layers were dried over anhydrous Na₂SO₄, filtered and concentrated under reduced pressure. The crude was purified by combi-flash chromatography to afford methyl 4-cyano-5-fluoro-2-methoxybenzoate 345 (5.5 g, 75%) as a brown solid. TLC: 25% EtOAc/Heptane (R_{f}: 0.4). LCMS calculated for C10H8FNO3: 209.05; Found: 209.8 (M+1).

### Synthesis of 2-fluoro-4-(hydroxymethyl)-5-methoxybenzonitrile (346)

To a stirred solution of compound 345 (6 g, 28.685 mmol) in THF (60 mL) was added 2M LiBH₄ in THF (43 mL, 43.027 mmol) at room temperature. The reaction mixture was stirred at 60° C. for 2 h. After completion (monitored by TLC), the mixture was quenched with ice-cold saturated NH₄Cl and extracted with EtOAc. The combined organic layers were washed with brine, dried over anhydrous Na₂SO₄, filtered and concentrated under reduced pressure to afford title compound 346 (3.75 g, 70.3%) as a white solid. TLC: 40% EtOAc/Heptane, (R_{f}: 0.45). ¹H NMR (401 MHz, DMSO-d₆) δ=7.46 (d, J=5.1 Hz, 1H), 7.40 (d, J=9.8 Hz, 1H), 5.49 (t, J=5.5 Hz, 1H), 4.52 (d, J=5.6 Hz, 2H), 3.83 (s, 3H).

### Synthesis of 4-cyano-5-fluoro-2-methoxybenzyl methanesulfonate (347)

To a stirred solution of compound 346 (3 g, 16.56 mmol) in DCM (30 mL) were added MsCl (1.54 mL, 19.872 mmol) and Et₃N (4.65 mL, 33.120 mmol) at 0° C. Then reaction mixture was warm to room temperature and stirred for 2 h at room temperature. After completion, the mixture was quenched with water and extracted with DCM. The combined organic layers were dried over anhydrous Na₂SO₄, filtered and concentrated under reduced pressure to afford title compound 347 (4 g, 48.17%) as a white solid. TLC: 60% EtOAc/Heptane, (R_{f}: 0.7). ¹H NMR (400 MHz, CHLOROFORM-d) 6=7.12-6.96 (m, 2H), 4.63 (s, 2H), 3.92 (s, 3H), 3.12 (s, 3H)

### Synthesis of tert-butyl ((1-(4-cyano-5-fluoro-2-methoxybenzyl)-1H-pyrazol-4-yl)methyl)carbamate (348)

To a stirred solution of compound 347 (4 g, 15.429 mmol) in CH₃CN (40 mL) were added Cs₂CO₃ (15 g, 46.287 mmol) and compound 19 (3.65 g, 18.515 mmol) at 0° C. The reaction mixture stirred at 70° C. for 2 h. After completion (monitored by TLC), the mixture was quenched with ice-cold water and extracted with EtOAc. The combined organic layers were washed with brine, dried over anhydrous Na₂SO₄, filtered and concentrated under reduced pressure. The crude was purified by combi-flash chromatography to afford title compound 348 (2.5 g, 37%) as a pale-yellow liquid. TLC: 60% EtOAc/Heptane, (R_{f}: 0.6). LCMS calculated for C18H21FN4O3: 360.16; Found: 361.1 (M+1).

### Synthesis of tert-butyl ((1-((3-amino-5-methoxybenzo[d]isoxazol-6-yl)methyl)-1H-pyrazol-4-yl)methyl)carbamate (349)

To a stirred solution of compound 348 (2 g, 5.549 mmol) in DMF (20 mL) were added acetohydroxamic acid (1.7 g, 22.20 mmol) and K₂CO₃ (4.6 g, 33.30 mmol). The reaction mixture was stirred at 60° C. for 16 h. After completion, the mixture was quenched with cold water and extracted with EtOAc. The combined organic layers were dried over anhydrous Na₂SO₄ and concentrated under reduced pressure. The crude was purified by combi-flash chromatography to afford title compound 349 (1.2 g, 52%) as an off white solid. LCMS calculated for C18H23N5O4: 373.18; Found: 374.1 (M+1).

### Synthesis of tert-butyl ((1-((5-methoxy-3-((2-methoxyphenyl)sulfonamido)benzo[d]isoxazol-6-yl)methyl)-1H-pyrazol-4-yl)methyl)carbamate (350)

To a stirred solution of compound 349 (0.6 g, 1.607 mmol) in THF (6 mL) were added KOtBu in THF (4 mL) and 2-methoxybenzenesulfonyl chloride (0.59 g, 2.892 mmol) at 0° C. The reaction mixture was stirred at room temperature for 16 h. After completion, the mixture was quenched with water and extracted with EtOAc. The combined organic layers were dried over anhydrous Na₂SO₄ and concentrated under reduced pressure. The crude was purified by combi-flash chromatography to afford title compound 350 (0.4 g, 33.47%) as an off white solid. LCMS calculated for C25H29N5O7S: 543.18; Found: 544.4 (M+1).

### Synthesis of N-(6-((4-(aminomethyl)-1H-pyrazol-1-yl)methyl)-5-methoxybenzo[d]isoxazol-3-yl)-2-methoxybenzenesulfonamide hydrochloride (351)

To a stirred solution of compound 350 (0.4 g, 0.735 mmol) in DCM (4 mL) was added 4M HCl in 1,4-Dioxane (4 mL, 16 mmol) at 0° C. The reaction mixture was stirred at room temperature for 1 h. After completion, the mixture was concentrated, triturated with Et₂O and dried under reduced pressure to afford title compound 351 (0.3 g, 85.65%) as a yellow solid. LCMS calculated for C20H21N5O5S: 443.13; Found: 442.8 (M−1).

### Synthesis of methyl ((1-((5-methoxy-3-((2-methoxyphenyl)sulfonamido)benzo[d]isoxazol-6-yl)methyl)-1H-pyrazol-4-yl)methyl)carbamate (Syn. Ex. 145)

To a stirred solution compound 351 (0.12 g, 0.271 mmol) in DCM (2 mL) were added Et₃N (0.117 mL, 0.813 mmol) and ClCO₂Me (0.025 mL, 0.325 mmol) at 0° C. The reaction mixture was stirred at 0° C. for 10 min. After completion (monitored by TLC), the mixture was quenched with ice-cold water and extracted with DCM. The combined organic layers were dried over anhydrous Na₂SO₄, filtered and concentrated under reduced pressure. The crude was purified by prep. HPLC to afford methyl ((1-((5-methoxy-3-((2-methoxyphenyl)sulfonamido)benzo[d]isoxazol-6-yl)methyl)-1H-pyrazol-4-yl)methyl)carbamate Syn. Ex. 145 (20 mg, 14.68%) as a white solid.

### Example 146

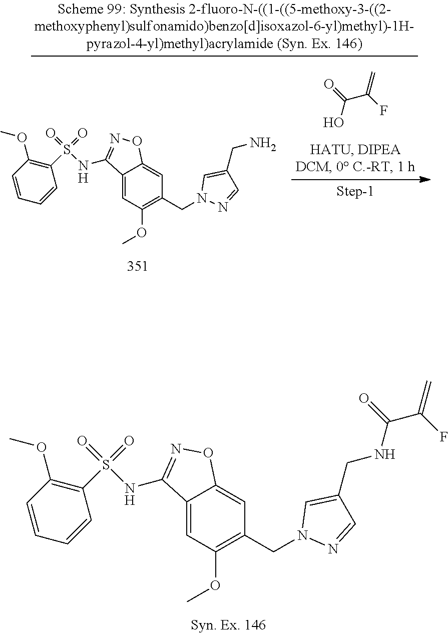

To a stirred solution of compound 351 (0.1 g, 0.225 mmol) in DCM (4 mL) were added DIPEA (0.117 mL, 0.6764 mmol), HATU (0.13 g, 0.338 mmol) and 2-fluoroacrylic acid (2) (0.024 g, 0.2706 mmol). The reaction mixture was stirred at room temperature for 1 h. After completion, the mixture was quenched with ice-cold water and extracted with DCM. The combined organic layers were dried over anhydrous Na₂SO₄, filtered and concentrated under reduced pressure. The crude was purified by prep. HPLC to afford 2-fluoro-N-((1-((5-methoxy-3-((2-methoxyphenyl)sulfonamido)benzo[d]isoxazol-6-yl)methyl)-1H-pyrazol-4-yl)methyl) acrylamide Syn. Ex. 146 (35 mg, 29.84%) as an off white solid.

### Example 147

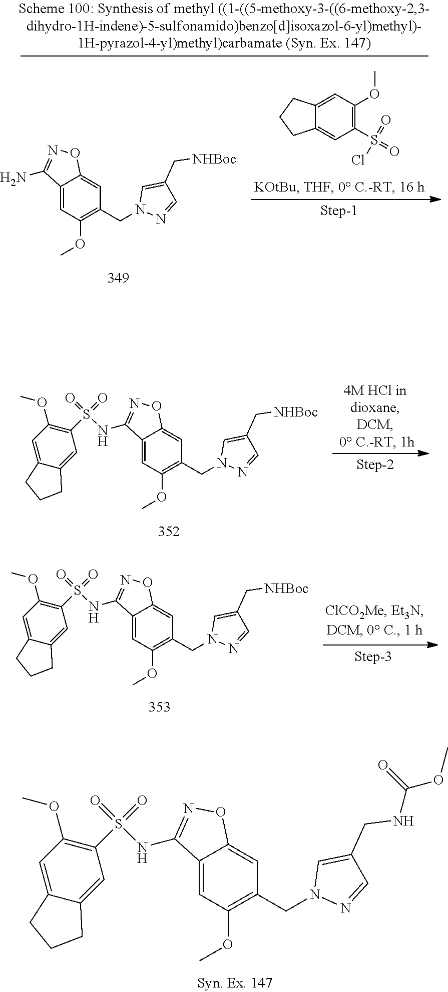

### Synthesis of tert-butyl ((1-((5-methoxy-3-((6-methoxy-2,3-dihydro-1H-indene)-5-sulfonamido)benzo[d]isoxazol-6-yl)methyl)-1H-pyrazol-4-yl)methyl)carbamate (352)

To a stirred solution of compound 349 (0.3 g, 0.803 mmol) in THF (4 mL) were added 1M KOtBu in THF (2 mL, 3.214 mmol) and 6-methoxy-2,3-dihydro-1H-indene-5-sulfonyl chloride (0.35 g, 1.446 mmol) at 0° C. The reaction mixture was stirred at room temperature for 16 h. After completion (monitored by TLC), the mixture was quenched with water and extracted with EtOAc. The combined organic layers were dried over anhydrous Na₂SO₄ and concentrated under reduced pressure. The crude was purified by combi-flash chromatography to afford title compound 352 (0.15 g, 27.73%) as an off white solid. LCMS calculated for C28H33N5O7S: 583.21; Found: 584.1 (M+1).

### Synthesis of N-(6-((4-(aminomethyl)-1H-pyrazol-1-yl)methyl)-5-methoxybenzo[d]isoxazol-3-yl)-6-methoxy-2,3-dihydro-1H-indene-5-sulfonamide hydrochloride (353)

To a stirred solution of compound 352 (0.12 g, 0.2056 mmol) in DCM (2 mL) was added 4M HCl in 1,4-Dioxane (1.2 mL) at 0° C. The reaction mixture was stirred at room temperature for 1 h. After completion (monitored by TLC), the mixture was concentrated under reduced pressure, triturated with Et₂O and concentrated under reduced pressure to afford title compound 353 (80 mg, 73.49%) as a pale yellow solid. LCMS calculated for C23H25N5O5S: 483.16; Found: 482.1 (M−1).

### Synthesis of methyl ((1-((5-methoxy-3-((6-methoxy-2,3-dihydro-1H-indene)-5-sulfonamido)benzo[d]isoxazol-6-yl)methyl)-1H-pyrazol-4-yl)methyl)carbamate (Syn. Ex. 147)

To a stirred solution compound 353 (0.07 g, 0.1448 mmol) in DCM (4 mL) were added Et₃N (0.062 mL, 0.4343 mmol) and ClCO₂Me (0.0135 mL, 0.1737 mmol) at 0° C. The reaction mixture was stirred at 0° C. for 10 min. After completion (monitored by TLC), the mixture was quenched with ice-cold water and extracted with DCM. The combined organic layers were dried over anhydrous Na₂SO₄, filtered and concentrated under reduced pressure. The crude was purified by prep. HPLC to afford methyl ((1-((5-methoxy-3-((6-methoxy-2,3-dihydro-1H-indene)-5-sulfonamido)benzo[d]isoxazol-6-yl)methyl)-1H-pyrazol-4-yl)methyl)carbamate Syn. EX. 147 (10 mg, 12.55%) as a white solid.

### Example 148

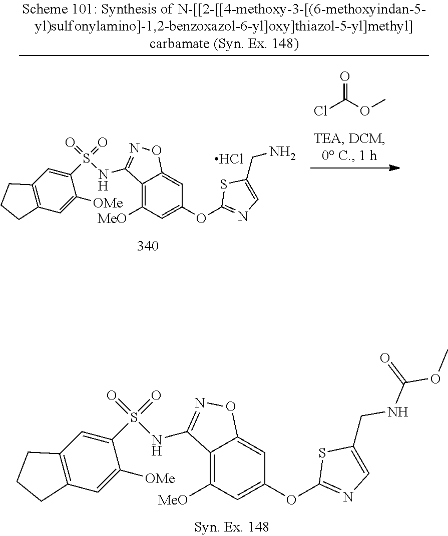

To a stirred solution of compound 340 (67 mg, 0.133 mmol) in DCM (2 mL) was added Triethylamine (0.052 mL, 0.399 mmol) and methyl carbonochloridate (0.013 g, 0.1466 mmol) at 0° C. and was stirred for 30 min. After completion (monitored by TLC), reaction mixture was quenched with ice cold water and extracted with DCM. Combined organic layer was washed with brine solution, dried over sodium sulphate, filtered and concentrated under reduced pressure. The crude was purified by prep-HPLC to afford N-[[2-[[4-methoxy-3-[(6-methoxyindan-5-yl)sulfonylamino]-1,2-benzoxazol-6-yl]oxy]thiazol-5-yl]methyl]carbamate (Syn. Ex. 148; 11 mg, 0.019 mmol, 14.68% yield) as an off white solid.

### Example 149

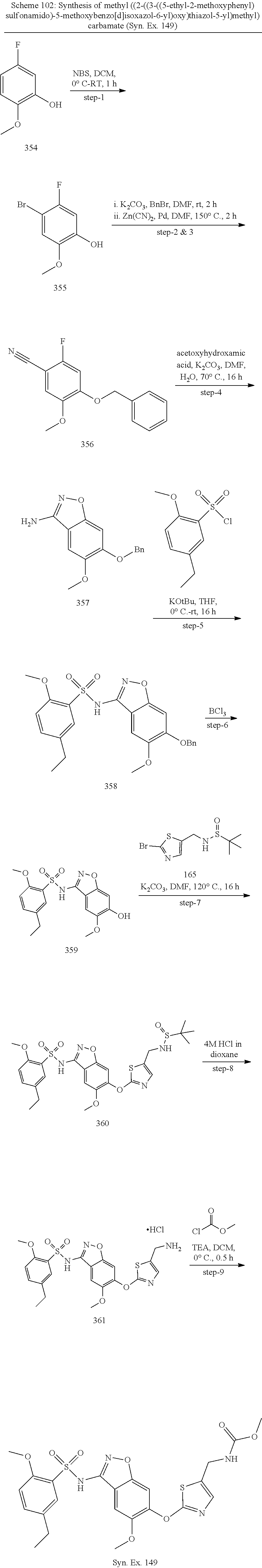

### Synthesis of 4-bromo-5-fluoro-2-methoxyphenol (355)

To a stirred solution of compound 354 (10.0 g, 70.4 mmol) in DCM (120 mL) was added NBS (12.53 g, 70.4 mmol, 98 mass %) portion wise over 10 min. The reaction mixture continued to stir at room temperature for 1 h. After completion (monitored by TLC), the mixture was quenched with ice cold water and was extracted with DCM. The combined organic layer was washed with brine solution and was dried over sodium sulphate, filtered and concentrated under reduced pressure. The crude compound purified by combi flash to afford title compound 355 (13.3 g, 38.5 mmol, 54.7% Yield) as a white solid. LCMS calculated for C7H6BrFO2: 221.02; Found: 220.8 (M−1). Regichemistry of bromine addition was confirmed by NMR.

### Synthesis of 1-benzyloxy-4-bromo-5-fluoro-2-methoxy-benzene (356)

To a stirred solution of compound 355 (13.2 g, 59.7 mmol) in acetonitrile (150 mL) was added potassium carbonate (24.8 g, 179 mmol) and benzyl bromide (11.7 g, 65.7 mmol) at 0° C. The reaction was stirred at room temperature for 16 h. After completion (monitored by TLC), the mixture was quenched with ice cold water and was extracted with ethyl acetate. The combined organic layer was washed with brine solution, dried over sodium sulphate, filtered and concentrated. The crude was purified by combi flash to afford title compound (9.1 g, 29 mmol, 48%) as a white solid. LCMS calculated for C15H12FNO2: 257.085; Found: 258.10 (M+1). This intermediate (5.0 g, 16 mmol) was dissolved in DMF (50 mL) and zinc cyanide (24 mmol) was added. The reaction was purged with nitrogen for 10 min followed by addition of Xantphos (0.96 g, 1.6 mmol) and Pd₂(dba)₃ (0.74 g, 0.80 mmol). The reaction mixture was stirred at 140° C. for 16 h. After completion (monitored by LCMS), the mixture quenched with ice cold water and was extracted with ethyl acetate. The combined organic layer was washed with brine solution, dried over sodium sulphate, filtered and concentrated under reduced pressure. The crude compound purified by combi flash to afford title compound 356 (1.6 g, 5.3 mmol, 33%) as a brown solid. LCMS calculated for C15H12FNO2: 257.3; Found: 256.0 (M−1).

### Synthesis of 6-benzyloxy-5-methoxy-1,2-benzoxazol-3-amine (357)

To a solution of compound 356 (1.60 g, 6.22 mmol) in DMF (35 mL) was added acetohydroxamic acid (1.43 g, 18.7 mmol) and potassium carbonate (3.44 g, 24.9 mmol). The reaction mixture was stirred at 70° C. for 16 h. After completion, the mixture was quenched with ice-cold water and extracted with ethyl acetate. Combined organic layer was washed with brine, dried over sodium sulphate, filtered and concentrated. The crude compound was triturated with diethyl ether to afford title compound 357 (1.0 g, 3.3 mmol, 53% Yield) as a white solid. LCMS calculated for C15H14N2O3: 270.3; Found: 271.1 (M+1).

### Synthesis of —{N}-(6-benzyloxy-5-methoxy-1,2-benzoxazol-3-yl)-5-ethyl-2-methoxy-benzenesulfonamide (358)

To a stirred solution of compound 357 in THF (15 mL) was added 1M solution of potassium tert-butoxide in THF (15.0 mL, 15.0 mmol) at 0° C. and stirred for 10 min. followed by addition of 5-ethyl-2-methoxy-benzenesulfonyl chloride (1.76 g, 7.49 mmol). The reaction mixture was slowly warmed to room temperature and stirred for 16 h. After completion (monitored by TLC), the mixture was quenched with ice-cold water and extracted with ethyl acetate. Combined organic layer was washed with brine solution, dried over sodium sulphate filtered and concentrated under reduced pressure. The crude was purified by combi flash to afford title compound 358 (1.2 g, 2.3 mmol, 46% Yield) as a brown solid. LCMS calculated for C24H24N2O6S: 468.5; Found: 469.3 (M+1).

### Synthesis of 5-ethyl-—{N}-(6-hydroxy-5-methoxy-1,2-benzoxazol-3-yl)-2-methoxy-benzenesulfonamide (359)

To a stirred solution of compound 358 (1.75 g, 3.74 mmol) in DCM (50.00 mL) was added 1M boron trichloride solution in toluene (26 mL, 26.1 mmol) at −78° C. The reaction mixture was stirred at −78° C. for 2 h. After completion (monitored by TLC), the mixture was quenched with ice-cold sodium bicarbonate solution and extracted with DCM. The combined organic layer was washed with brine solution, dried over sodium sulphate, filtered and concentrated under reduced pressure. The crude compound was triturated with diethyl ether to afford title compound 359 (1.1 g, 2.8 mmol, 74% Yield) as a brown solid. LCMS calculated for C17H18N2O6S: 378.4; Found: 379.1 (M+1)

### Synthesis of —{N}-[6-[5-[(-{tert}-butylsulfinylamino)methyl]thiazol-2-yl]oxy-5-methoxy-1,2-benzoxazol-3-yl]-5-ethyl-2-methoxy-benzenesulfonamide (360)

To a stirred solution of compound 359 (500 mg, 1.321 mmol) in DMF (5 mL) was added potassium carbonate (0.369 gr, 2.643 mmol) and compound 165 (0.589 gm, 1.982 mmol) at room temperature. The reaction mixture was stirred at 120° C. for 24 h. After completion (monitored by TLC), the mixture was quenched with ice-cold water and extracted with EtOAc. The combined organic layer was dried over anhydrous Na₂SO₄ and concentrated under reduced pressure. The crude compound was purified by combi flash to afford title compound 360 (100 mg, 0.135 mmol, 10.22%) as a brown solid. LCMS calculated for C25H30N4O7S3: 594.7; Found: 595.5 (M+1).

### Synthesis of —{N}-[6-[5-(aminomethyl)thiazol-2-yl]oxy-5-methoxy-1,2-benzoxazol-3-yl]-5-ethyl-2-methoxy-benzenesulfonamide (361)

To a stirred solution of compound 360 (100 mg, 0.1682 mmol) in DCM (5 mL) was added 4M HCl in dioxane (0.21 mL, 0.8408 mmol) at 0° C. Then reaction mixture was stirred at room temperature for 2 h. After completion (monitored by TLC), the reaction was concentrated and triturated with diethyl ether to afford title compound 361 (82 mg, 0.1549 mmol, 92.11% Y) as a brown solid. LCMS calculated for C21H22N4O6S2: 490.6; Found: 489.0 (M−1).

### Synthesis of methyl —{N}—[[2-[[3-[(5-ethyl-2-methoxy-phenyl)sulfonylamino]-5-methoxy-1,2-benzoxazol-6-yl]oxy]thiazol-5-yl]methyl]carbamate (Syn. Ex. 149)

To a stirred solution of compound 361 (80 mg, 0.15 mmol) in DCM (4 mL) was added triethylamine (0.064 mL, 0.455 mmol) in (0.2 mL of DCM) and methyl carbonochloridate (0.0129 mL, 0.166 mmol) in (0.2 mL of DCM) simultaneously while maintaining (pH-7-8) at 0° C. After completion (monitored by TLC), the mixture was quenched with ice-cold water and extracted with DCM. The combined organic layer was washed with brine solution, dried over sodium sulphate, filtered and concentrated under reduced pressure. The crude compound was purified by prep-HPLC purification to afford title compound (Syn. Ex. 149; 21 mg, 0.037 mmol, 24.67%) as an off white solid.

### Example 150

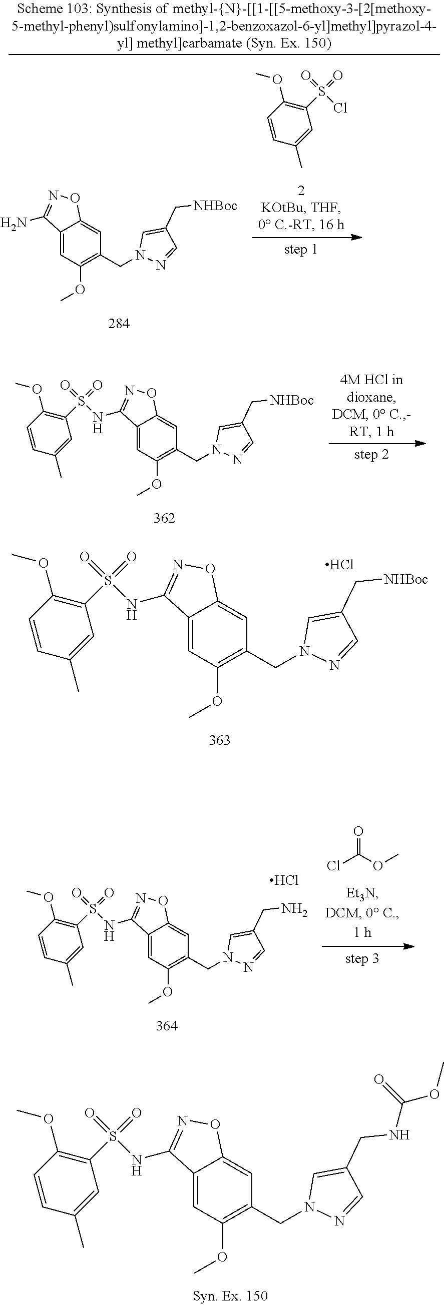

### Synthesis of tert-butyl ((1-((5-methoxy-3-((2-methoxy-5-methylphenyl)sulfonamido)benzo[d]isoxazol-6-yl)methyl)-1H-pyrazol-4-yl)methyl)carbamate (362)

To a stirred solution of compound 284 (200 mg, 0.535 mmol) in THF (4 mL) was added 1.6M potassium tert-butoxide in THF (1.0 mL, 1.607 mmol) at 0° C. The reaction was stirred for 5 min followed by addition of 2-methoxy-5-methyl-benzenesulfonyl chloride (200 mg, 0.910 mmol) at 0° C. The reaction mixture was stirred at RT for 16 h. After completion (monitored by TLC), the mixture was quenched with aqueous solution of citric acid and extracted with EtOAc. The combined organic layer was washed with brine, dried over anhydrous Na₂SO₄, filtered and concentrated under reduced pressure to afford title compound 362 (220 mg, 0.211 mmol, 39.53%) as pale yellow solid. LCMS calculated for C26H31N5O7S: 557.6; Found: 558.0 (M+1).

### Synthesis of N-(6-((4-(aminomethyl)-1H-pyrazol-1-yl)methyl)-5-methoxybenzo[d]isoxazol-3-yl)-2-methoxy-5-methylbenzenesulfonamide (363)

To a stirred solution of compound 362 (200 mg, 0.358 mmol) in DCM (2 mL) was added 4M HCl in 1,4-dioxane (2 mL, 8 mmol). The reaction mixture was stirred at room temperature for 1 h. After completion (monitored by TLC), then reaction mixture was concentrated under reduced pressure. The crude compound was triturated with diethyl ether to afford title compound 363 (220 mg, 0.270 mmol, 75.48% Yield) as a brown solid. LCMS calculated for C21H23N5O5S: 457.5; Found: 456.1 (M−1).

### Synthesis of N-(6-((4-(aminomethyl)-1H-pyrazol-1-yl)methyl)-5-methoxybenzo[d]isoxazol-3-yl)-2-methoxy-5-methylbenzenesulfonamide (Syn. Ex. 150)

To a solution of compound 363 (150 mg, 0.3279 mmol) in DCM (5 mL) was added triethylamine (0.141 mL, 0.983 mmol) in 0.3 mL of DCM and methyl carbonochloridate (0.0381 mL, 0.491 mmol) in (0.3 mL of DCM) was added simultaneously while maintaining (pH-7-8) at 0° C. The reaction was stirred for 10 min. After completion (monitored by TLC), the mixture was quenched with ice-cold water and extracted with DCM. The combined organic layer was washed with brine solution, dried over sodium sulphate, filtered and concentrated under reduced pressure. The crude compound was purified by prep-HPLC purification to afford title compound (Syn. Ex. 150; 8 mg, 0.0147 mmol, 4.51%) as an off-white solid.

### Example 151

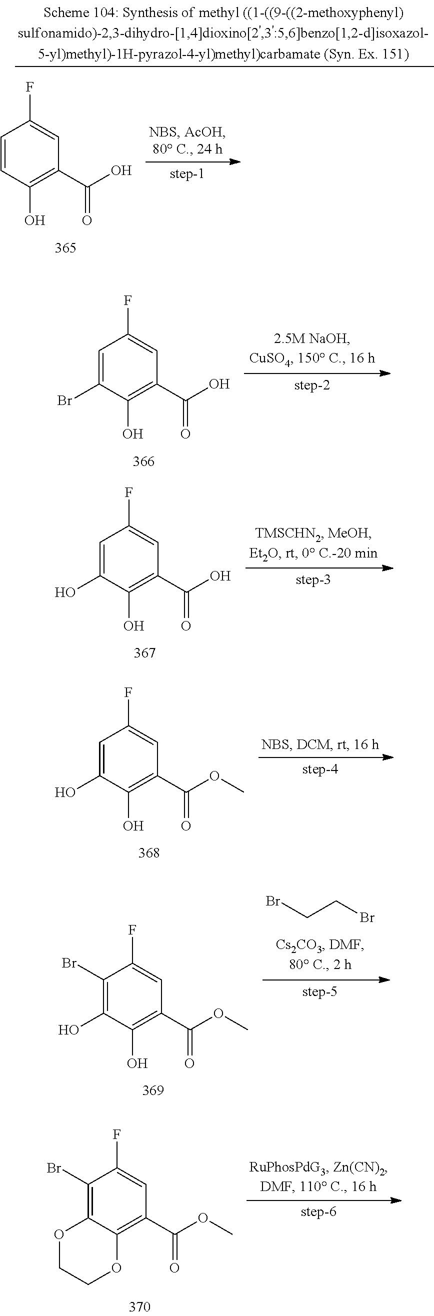

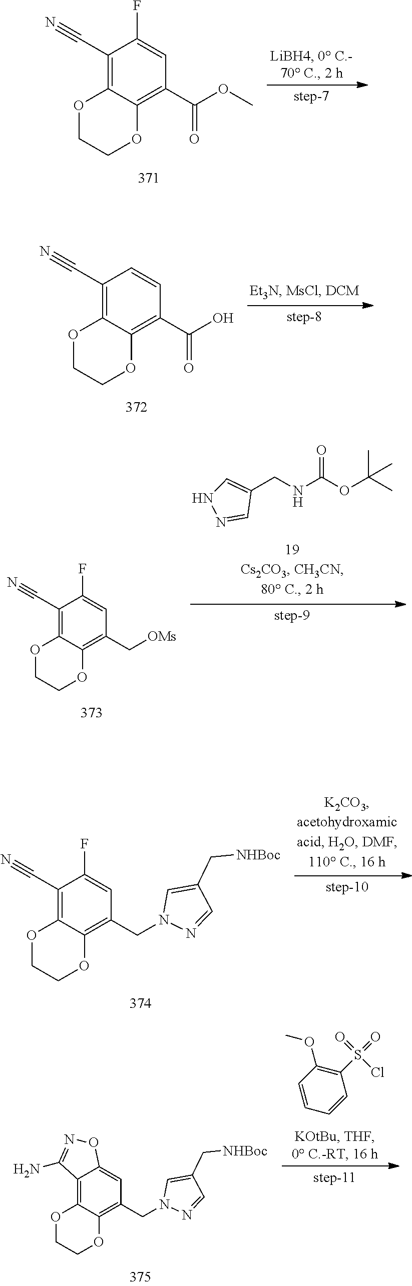

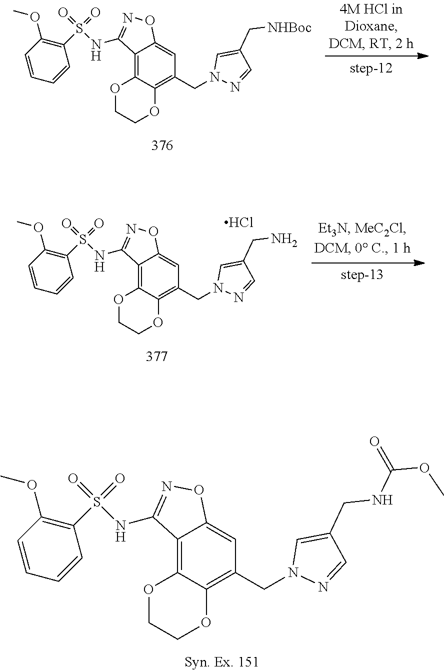

### Synthesis of 3-bromo-5-fluoro-2-hydroxy-benzoic acid (366)

To a stirred solution of compound 365 (30.0 g, 192 mmol) in acetic acid (300 mL) was added N-bromosuccinimide (34.9 g, 192 mmol) at room temperature. The reaction mixture was stirred at 80° C. for 24 h. After completion, the mixture was quenched with ice cold water and was extracted with ethyl acetate. The combined organic layer was washed with brine solution and dried over sodium sulphate, filtered and concentrated under reduced pressure. The crude was triturated with 2% EA/Heptane to afford title compound 366 (30 g, 127.65 mmol, 66.4% Yield) as a brown solid. LCMS calculated for C7H4BrFO3: 235.01; Found: 232.8 (M−2).

### Synthesis of 5-fluoro-2,3-dihydroxy-benzoic acid (367)

To a solution of 2.5M sodium hydroxide in H₂O (210 mL, 521 mmol) was added copper (II) sulfate (0.238 g, 1.49 mmol). The mixture was stirred at room temperature for 1 h, then filtered. Compound 366 (17.5 g, 74.5 mmol) was added to the filtrate. The reaction mixture was stirred at 150° C. for 16 h. After completion, the reaction mixture was cooled and acidified with 2.0 M HCl to pH<2, then diluted with H₂O (500 mL) and was extracted with Ethyl acetate. The combined organic layer was washed with brine solution and dried over sodium sulphate filtered and concentrated under reduced pressure. The crude compound was triturated with 5% EA/Heptane to afford title compound 367 (9.2 g, 48 mmol, 64.0% Yield) as a brown solid. LCMS calculated for C7H5FO4:172.11; Found: 170.9 (M−1).

### Synthesis of 5-fluoro-2,3-dihydroxy-benzoic acid (368)

To a stirred solution of compound 367 (5.0 g, 29 mmol) in methanol (50 mL) and diethyl ether (20 mL) was added 2M (trimethylsilyl)diazomethane in hexanes (38 mL, 76 mmol) drop wise over 10 min at room temperature. The reaction was stirred at room temperature for 20 min. After completion (monitored by TLC), the reaction mixture was concentrated under reduced pressure. The crude compound purified by combi flash to afford title compound 368 (3.74 g, 19.6 mmol, 68% Yield) as a brown solid. LCMS calculated for C8H7FO4: 186.14; Found: 185.0 (M−1).

### Synthesis of methyl 4-bromo-5-fluoro-2,3-dihydroxy-benzoate (369)

To a stirred solution of compound 368 (5.0 g, 27 mmol) in DCM (150 mL) was added N-bromosuccinimide (5.9 g, 32 mmol) at 0° C. portion wise over 5 min. The reaction mixture was stirred at room temperature for 16 h. After completion (monitored by TLC), the mixture was quenched with ice cold water and extracted with DCM. The combined organic layer was washed with brine solution and dried over sodium sulphate filtered and concentrated under reduced pressure. The crude compound purified by combi flash to afford title compound 369 (3.2 g, 9.9 mmol, 37% Yield) as a brown solid. LCMS calculated for C8H6BrFO4: 265.03; Found: 266.1 (M+H).

### Synthesis of methyl 5-bromo-6-fluoro-2,3-dihydro-1,4-benzodioxine-8-carboxylate (370)

To a stirred solution of compound 369 (2.5 g, 9.4 mmol) in N,N-dimethylformamide (25.00 mL) was added cesium carbonate (6.2 g, 19 mmol) and 1,2-dibromoethane (1.3 mL, 15 mmol). The reaction mixture was stirred at 80° C. for 2 h. After completion (monitored by TLC), the mixture quenched with ice cold water and stirred for 10 min to afford a solid. The solid was filtered to afford title compound 370 (2.3 g, 7.0 mmol, 75% Yield) as a brown solid. LCMS calculated for C10H8BrFO4: 291.07; Found: 293.2 (M+2).

### Synthesis of methyl 5-cyano-6-fluoro-2,3-dihydro-1,4-benzodioxine-8-carboxylate (371)

To a stirred solution of compound 370 (3.00 g, 10.3 mmol) in N,N-dimethylformamide (50.00 mL) was added zinc cyanide (3.09 g, 25.8 mmol) and RuPhos Pd G3 (1.36, 1.55 mmol). The solution was purged with nitrogen for 3 min. The reaction mixture was stirred at 110° C. for 16 h. After completion (monitored by TLC), the reaction was quenched with ice cold water and extracted with ethyl acetate. The combined organic layer was washed with brine solution, dried over sodium sulphate, filtered and concentrated under reduced pressure. The crude compound purified by combi flash to afford title compound 371 (1.50 g, 6.32 mmol, 61.4% Yield) as a brown solid. LCMS calculated for C11H8FNO4: 237.044 found: 238.1 (M+1).

### Synthesis of 6-fluoro-8-(hydroxymethyl)-2,3-dihydro-1,4-benzodioxine-5-carbonitrile (372)

To a stirred solution of compound 371 (1.5 g, 6.3 mmol) in tetrahydrofuran (20 mL) was added 1M lithium borohydride in THF (13 mL, 13 mmol) at 0° C. The reaction mixture was stirred at 65° C. for 2 h. After completion (monitored by TLC), the reaction was quenched with sat. ammonium chloride solution and extracted with ethyl acetate. The combined organic layer was washed with brine solution, dried over sodium sulphate, filtered and concentrated under reduced pressure to afford title compound 372 (1.3 g, 5.2 mmol, 83% Yield) as a white solid. LCMS calculated for C10H8FNO3: 209.049 found 210.2 (M+H).

### Synthesis of (5-cyano-6-fluoro-2,3-dihydro-1,4-benzodioxin-8-yl)methyl methane sulfonate (373)

To a stirred solution of compound 372 (1.3 g, 6.2 mmol) in DCM (30 mL) was added triethylamine (4.4 mL, 31 mmol) and methanesulfonyl chloride (0.98 mL, 12 mmol) at 0° C. The reaction mixture was stirred at room temperature for 2 h. After completion (monitored by TLC), the reaction was quenched with ice cold water and extracted with DCM. The combined organic layer was washed with brine solution, dried over sodium sulphate, filtered and concentrated under reduced pressure to afford title compound 373 (1.3 g, 3.4 mmol, 55% Yield) as a brown semi solid. LCMS calculated for C11H10FNO5S: 287.026 found 288.2 (M+H).

### Synthesis of tert-butyl ((1-((8-cyano-7-fluoro-2,3-dihydrobenzo[b][1,4]dioxin-5-yl)methyl)-1H-pyrazol-4-yl)methyl)carbamate (374)

To a stirred solution compound 373 (1.3 g, 4.5 mmol) in acetonitrile (15.00 mL) was added cesium carbonate (3.0 g, 9.0 mmol) and stirred for 5 min. Compound (10) (1.3 g, 6.8 mmol) was added at room temperature. The reaction was stirred at 70° C. for 3 h. After completion (monitored by TLC), the reaction was concentrated under reduced pressure, quenched with ice cold water and extracted with ethyl acetate. The combined organic layer was washed with brine, dried over sodium sulphate, filtered and concentrated under reduced pressure. The crude was purified by combi flash to afford title compound 374 (460 mg, 1.03 mmol, 23% Yield) as a brown gummy liquid. LCMS calculated for C19H21FN404: 388.4; Found: 389.0 (M+1).

### Synthesis of tert-butyl ((1-((9-amino-2,3-dihydro-[1,4]dioxino[2′,3′:5,6]benzo[1,2-d]isoxazol-5-yl)methyl)-1H-pyrazol-4-yl)methyl)carbamate (375)

To a stirred solution of compound 374 (260.00 mg, 0.669 mmol) in N,N-dimethylformamide (5 mL) was added potassium carbonate (555 mg, 4.016 mmol), water (0.7 mL, 40 mmol) and acetoxy hydroxamic acid (150.8 mg, 2.008 mmol). The reaction mixture was stirred at 110° C. for 16 h. After completion (monitored by LCMS), the reaction was quenched with ice cold water and extracted with ethyl acetate. The combined organic layer was washed with brine, dried over sodium sulphate, filtered and concentrated under reduced pressure. The crude compound was purified by combi flash to afford title compound (375) (150.00 mg, 0.32 mmol, 48.10% Yield) as a brown solid. LCMS calculated for C19H23N5O5: 401.4; Found: 402.0 (M+1).

### Synthesis of tert-butyl ((1-((9-((2-methoxyphenyl)sulfonamido)-2,3-dihydro-[1,4]dioxino[2′,3′:5,6]benzo[1,2-d]isoxazol-5-yl)methyl)-1H-pyrazol-4-yl)methyl)carbamate (376)

To a stirred solution of compound 375 (225 mg, 0.5605 mmol) in THF (5 mL) was added 1.6 M potassium tert-butoxide in THF (1.1 mL, 1.682 mmol) at 0° C. The reaction was stirred for 5 min followed by addition of 2-methoxybenzenesulfonyl chloride (173.8 mg, 0.8408 mmol) at 0° C. The reaction was stirred at room temperature for 16 h. After completion (monitored by LCMS), the reaction was quenched with ice cold water and extracted with ethyl acetate. The combined organic layer was washed with brine, dried over sodium sulphate, filtered and concentrated under reduced pressure. The crude compound was purified by combi flash to afford title compound 376 (160 mg, 0.2799 mmol, 49.94% Yield) as a brown solid. LCMS calculated for C26H29N5O8S: 571.6; Found: 572.0 (M+1).

### Synthesis of N-(5-((4-(aminomethyl)-1H-pyrazol-1-yl)methyl)-2,3-dihydro-[1,4]dioxino[2′,3′:5,6]benzo[1,2-d]isoxazol-9-yl)-2-methoxybenzenesulfonamide.HCl (377)

To a stirred solution of compound 376 (80.00 mg, 0.1400 mmol) in DCM (2.0 mL) was added 4M hydrochloric acid in dioxane (0.17 mL, 0.6998 mmol) at 0° C. The reaction mixture was stirred at room temperature for 2 h. After completion (monitored by TLC), reaction was concentrated under reduced pressure. The crude compound was triturated with diethyl ether to afford title compound 377 (68.00 mg, 0.1370 mmol, 97.89% Yield) as a brown solid. LCMS calculated for C21H21N5O6S: 471.5; Found: 470.0 (M−1).

### Synthesis methyl ((1-((9-((2-methoxyphenyl)sulfonamido)-2,3-dihydro-[1,4]dioxino[2′,3′:5,6]benzo[1,2-d]isoxazol-5-yl)methyl)-1H-pyrazol-4-yl)methyl)carbamate (Syn. Ex. 151)

To a stirred solution of 377 (66 mg, 0.1400 mmol) in DCM (2 mL) was added Triethylamine (0.059 mL, 0.419 mmol) in dichloromethane (0.2 mL) and methyl carbonochloridate (0.012 mL, 0.154 mmol) in dichloromethane (0.2 mL) simultaneously while maintaining (pH-7-8) at 0° C. The reaction was stirred for 30 min. at 0° C. After completion (monitored by TLC), the mixture was quenched with ice-cold water and extracted with DCM. The combined organic layer was washed with brine solution and was dried over sodium sulphate, filtered and concentrated under reduced pressure and purified by preparative HPLC to afford Syn. Ex. 151 (28 mg, 0.139 mmol, 38% yield).

TABLE 1
Analytical data for compounds provided herein.

| | Mass Spec. Found | |
|---|---|---|
| | (m/z)/Mass Spec. | ¹H-NMR |
| Compound | Calculated | (400 MHz, DMSO-d₆, unless otherwise stated) |
| Syn. Ex. | 542.1 (M + 1)/ | δ 9.57 (s, 1H), 7.51 (t, J = 8.4 Hz, 1H), 7.43 (s, 1H), |
| 1 | 541.08 for | 7.38-7.31 (m, 1H), 7.27 (s, 1H), 7.19 (d, J = 7.6 Hz, |
| | C24H20ClN5O6S | 1H), 7.09-7.00 (m, 2H), 6.79 (d, J = 8.5 Hz, 2H), 4.02 |
| | | (s, 3H), 3.79 (s, 6H), 3.61 (br d, J = 2.4 Hz, 3H), 3.29- |
| | | 3.23 (m, 3H), 3.20 (br s, 2H), 2.40-2.33 (m, 2H), 1.01 |
| | | (t, J = 7.4 Hz, 3H). |
| Syn. Ex. | 542.1 (M + 1)/ | δ 11.36-10.26 (m, 1H), 7.82-7.56 (m, 3H), 7.21 (br s, |
| 2 | 541.08 for | 1H), 6.97-6.68 (m, 2H), 5.41 (br s, 2H), 4.71 (br s, |
| | C24H20ClN5O6S | 2H), 4.56 (br d, J = 5.6 Hz, 1H), 4.43 (br s, 2H), 4.01- |
| | | 3.72 (m, 6H). |
| Syn. Ex. | 512.1 (M + 1)/ | δ 10.14 (s, 1H), 7.80 (br d, J = 6.8 Hz, 1H), 7.62 (br t, |
| 3 | 511.15 for | J = 7.6 Hz, 1H), 7.34 (d, J = 3.0 Hz, 1H), 7.19 (br d, J = |
| | C24H25N5O6S | 8.1 Hz, 1H), 7.09 (br t, J = 7.4 Hz, 1H), 6.90 (br d, J = |
| | | 17.6 Hz, 1H), 6.77 (br d, J = 5.6 Hz, 1H), 5.38 (br d, |
| | | J = 2.6 Hz, 2H), 4.63-4.46 (m, 2H), 4.34 (br d, J = 4.3 |
| | | Hz, 2H), 3.84 (d, J = 4.9 Hz, 3H), 3.79 (d, J = 4.5 Hz, |
| | | 3H), 2.38-2.21 (m, 2H), 1.00 (br t, J = 7.3 Hz, 3H). |
| Syn. Ex. | 538.1 (M + 1)/ | δ 10.20 (s, 1H), 7.35 (d, J = 0.9 Hz, 1H), 7.31 (d, J = |
| 4 | 537.13 for | 3.1 Hz, 1H), 7.22 (dd, J = 0.7, 3.1 Hz, 1H), 7.21-7.18 |
| | C25H23N5O7S | (m, 1H), 7.16 (s, 1H), 7.13 (d, J = 0.7 Hz, 1H), 5.39 (d, |
| | | J = 9.9 Hz, 2H), 4.77 (s, 1H), 4.65 (s, 1H), 4.57 (d, J = |
| | | 4.2 Hz, 1H), 4.44 (s, 1H), 4.39 (s, 1H), 3.85 (d, J = 2.1 |
| | | Hz, 3H), 3.74 (s, 3H), 3.73 (d, J = 0.6 Hz, 3H). |
| Syn. Ex. | 538.28 (M + 1)/ | δ 10.19 (s, 1H), 7.76 (s, 1H), 7.30 (d, J = 3.1 Hz, 1H), |
| 5 | 537.13 for | 7.23-7.18 (m, 1H), 7.16-7.11 (m, 1H), 6.93 (s, 1H), |
| | C25H23N5O7S | 6.81 (s, 1H), 5.42 (s, 2H), 4.71 (d, J = 1.5 Hz, 2H), 4.56 |
| | | (d, J = 6.5 Hz, 1H), 4.42 (s, 2H), 3.85 (s, 3H), 3.74 (d, |
| | | J = 4.2 Hz, 6H). |
| Syn. Ex. | 522.0 (M + 1)/ | δ 10.12 (s, 1H), 7.89-7.77 (m, 2H), 7.62 (br t, J = 7.1 |
| 6 | 521.10 for | Hz, 1H), 7.45 (s, 1H), 7.29-7.25 (m, 1H), 7.19 (br d, |
| | C21H23N5O7S2 | J = 8.1 Hz, 1H), 7.09 (br t, J = 7.7 Hz, 1H), 6.85 (s, 1H), |
| | | 6.75 (s, 1H), 5.39 (s, 2H), 4.01 (d, J = 5.9 Hz, 2H), 3.80 |
| | | (d, J = 16.9 Hz, 6H), 2.82 (s, 3H). |
| Syn. Ex. | 502.28 (M + 1)/ | δ 10.11 (s, 1H), 7.80 (dd, J = 1.7, 7.8 Hz, 1H), 7.71 (s, |
| 7 | 501.13 for | 1H), 7.65-7.59 (m, 1H), 7.43 (br s, 1H), 7.37 (s, 1H), |
| | C22H23N5O7S | 7.19 (d, J = 8.3 Hz, 1H), 7.09 (t, J = 7.5 Hz, 1H), 6.85 |
| | | (s, 1H), 6.74 (s, 1H), 5.37 (s, 2H), 4.01 (d, J = 5.9 Hz, |
| | | 2H), 3.82 (s, 3H), 3.78 (s, 3H), 3.52 (s, 3H). |
| Syn. Ex. | 514.1 (M + 1)/ | δ 10.02 (br s, 1H), 8.25-7.98 (m, 1H), 7.72 (s, 1H), |
| 8 | 513.17 for | 7.62 (d, J = 2.3 Hz, 1H), 7.53-7.42 (m, 1H), 7.37 (s, |
| | C24H27N5O6S | 1H), 7.10 (br d, J = 8.5 Hz, 1H), 6.92-6.71 (m, 2H), |
| | | 5.36 (s, 2H), 4.06 (d, J = 5.5 Hz, 2H), 3.83 (s, 3H), 3.74 |
| | | (s, 3H), 2.60 (q, J = 7.5 Hz, 2H), 1.79 (s, 3H), 1.19- |
| | | 1.10 (m, 3H). |
| Syn. Ex. | 564.1 (M + 1)/ | δ 10.03 (s, 1H), 7.61 (dd, J = 1.8, 8.3 Hz, 2H), 7.45 (br |
| 9 | 563.18 for | d, J = 6.1 Hz, 1H), 7.11 (br d, J = 8.3 Hz, 1H), 6.88 (br |
| | C28H29N5O6S | s, 1H), 6.74 (br s, 1H), 5.27 (s, 2H), 4.56 (s, 1H), 3.92- |
| | | 3.76 (m, 5H), 3.76-3.72 (m, 3H), 3.62-3.52 (m, 2H), |
| | | 2.86-2.79 (m, 1H), 2.74-2.66 (m, 2H), 2.63-2.55 (m, |
| | | 3H), 1.14 (t, J = 7.5 Hz, 3H). |
| Syn. Ex. | 552.1 (M + 1)/ | δ 9.83-9.70 (m, 1H), 7.80 (s, 1H), 7.72 (d, J = 8.5 Hz, |
| 10 | 551.11 for | 1H), 7.45 (s, 1H), 7.27 (t, J = 5.9 Hz, 1H), 6.83 (br s, |
| | C22H25N5O8S2 | 1H), 6.74 (br s, 1H), 6.66-6.60 (m, 2H), 5.39 (s, 2H), |
| | | 4.01 (d, J = 6.0 Hz, 2H), 3.87 (s, 3H), 3.82 (s, 3H), 3.77 |
| | | (s, 3H), 2.81 (s, 3H). |
| Syn. Ex. | 550.2 (M + 1)/ | δ 10.01-9.27 (m, 1H), 7.66 (s, 1H), 7.45 (br d, J = 8.6 |
| 11 | 549.17 for | Hz, 1H), 7.33 (s, 1H), 7.11 (br dd, J = 3.3, 8.4 Hz, 1H), |
| | C27H27N5O6S | 6.97 (s, 1H), 6.77 (br d, J = 4.4 Hz, 1H), 5.38 (br d, J = |
| | | 8.8 Hz, 2H), 4.63 (br s, 2H), 4.38 (br d, J = 11.0 Hz, |
| | | 2H), 3.88 (d, J = 4.2 Hz, 3H), 3.79 (d, J = 4.9 Hz, 3H), |
| | | 2.62 (q, J = 7.6 Hz, 2H), 2.16-1.91 (m, 3H), 1.17 (t, |
| | | J = 7.5 Hz, 3H). |
| Syn. Ex. | 550.03(M + 1)/ | δ 10.03 (s, 1H), 7.74 (s, 1H), 7.62 (d, J = 2.1 Hz, 1H), |
| 12 | 549.17 for | 7.57-7.42 (m, 1H), 7.11 (d, J = 8.4 Hz, 1H), 6.93 (s, |
| | C27H27N5O6S | 1H), 6.81 (s, 1H), 5.41 (s, 2H), 4.67 (s, 2H), 4.39 (s, |
| | | 2H), 3.84 (s, 3H), 3.75 (s, 3H), 2.60 (q, J = 7.7 Hz, 2H), |
| | | 2.09-2.01 (m, 3H), 1.14 (t, J = 7.5 Hz, 3H). |
| Syn. Ex. | 556.12 (M + 1)/ | δ 9.48 (br s, 1H), 7.66 (s, 1H), 7.44 (br d, J = 8.3 Hz, |
| 13 | 555.16 for | 1H), 7.33 (br s, 1H), 7.11 (br d, J = 8.6 Hz, 1H), 6.95 |
| | C26H26FN5O6S | (br s, 1H), 6.77 (s, 1H), 5.51 (br d, J = 3.5 Hz, 1H), 5.38 |
| | | (s, 2H), 5.29 (br dd, J = 3.1, 17.3 Hz, 1H), 4.77-4.48 |
| | | (m, 4H), 3.87 (s, 3H), 3.78 (s, 3H), 2.62 (q, J = 7.5 Hz, |
| | | 2H), 1.17 (t, J = 7.5 Hz, 3H). |
| Syn. Ex. | 544.66 (M + 1)/ | δ 10.96-9.86 (m, 1H), 9.19-8.64 (m, 1H), 7.74 (s, |
| 14 | 543.16 for | 1H), 7.62 (d, J = 2.1 Hz, 1H), 7.45 (br d, J = 6.4 Hz, |
| | C25H26FN5O6S | 1H), 7.39 (s, 1H), 7.10 (br d, J = 8.6 Hz, 1H), 6.87 (br |
| | | s, 1H), 6.73 (br s, 1H), 5.57 (d, J = 3.4 Hz, 1H), 5.37 (s, |
| | | 2H), 5.23 (dd, J = 3.4, 15.8 Hz, 1H), 4.19 (d, J = 5.9 Hz, |
| | | 2H), 3.81 (s, 3H), 3.74 (s, 3H), 2.69-2.56 (m, 2H), 1.14 |
| | | (t, J = 7.6 Hz, 3H). |
| Syn. Ex. | 538.61 (M + 1)/ | δ 10.00 (br s, 1H), 8.77 (br t, J = 5.5 Hz, 1H), 7.71 (s, |
| 15 | 537.17 for | 1H), 7.62 (d, J = 2.1 Hz, 1H), 7.48-7.39 (m, 1H), 7.36 |
| | C26H27N5O6S | (s, 1H), 7.20-7.02 (m, 1H), 6.84 (br s, 1H), 6.72 (br s, |
| | | 1H), 5.36 (s, 2H), 4.09 (d, J = 5.8 Hz, 2H), 3.82 (s, 3H), |
| | | 3.74 (s, 3H), 2.67-2.57 (m, 2H), 1.93 (s, 3H), 1.14 (t, |
| | | J = 7.6 Hz, 3H). |
| Syn. Ex. | 544.44(M + 1)/ | δ 10.03 (s, 1H), 7.70 (s, 1H), 7.62 (d, J = 2.1 Hz, 1H), |
| 16 | 543.18 for | 7.46 (dd, J = 2.1, 8.6 Hz, 1H), 7.42-7.32 (m, 2H), 7.11 |
| | C25H29N5O7S | (d, J = 8.5 Hz, 1H), 6.85 (s, 1H), 6.74 (s, 1H), 5.37 (s, |
| | | 2H), 3.99 (dd, J = 6.4, 13.4 Hz, 4H), 3.82 (s, 3H), 3.74 |
| | | (s, 3H), 2.60 (q, J = 7.6 Hz, 2H), 1.13 (dt, J = 3.4, 7.3 |
| | | Hz, 6H). |
| Syn. Ex. | 530.4 (M + 1)/ | δ 10.01 (br s, 1H), 7.71 (s, 1H), 7.62 (d, J = 2.1 Hz, 1H), |
| 17 | 529.16 for | 7.43 (br d, J = 5.0 Hz, 2H), 7.37 (s, 1H), 7.10 (br d, J = |
| | C24H27N5O7S | 7.5 Hz, 1H), 6.85 (br s, 1H), 6.74 (br s, 1H), 5.37 (s, |
| | | 2H), 4.01 (d, J = 5.9 Hz, 2H), 3.82 (s, 3H), 3.74 (s, 3H), |
| | | 3.51 (s, 3H), 2.59 (q, J = 7.4 Hz, 2H), 1.14 (t, J = 7.6 |
| | | Hz, 3H). |
| Syn. Ex. | 594.71 (M + 1)/ | δ 10.43-9.74 (m, 1H), 7.79 (s, 1H), 7.62 (d, J = 2.3 Hz, |
| 18 | 593.16 for | 1H), 7.49-7.42 (m, 2H), 7.36 (t, J = 5.9 Hz, 1H), 7.10 |
| | C25H31N5O8S2 | (br d, J = 8.5 Hz, 1H), 6.85 (s, 1H), 6.76 (s, 1H), 5.39 |
| | | (s, 2H), 4.00 (d, J = 5.9 Hz, 2H), 3.83 (s, 3H), 3.74 (s, |
| | | 3H), 3.60-3.51 (m, 3H), 3.22-3.16 (m, 7H), 2.69- |
| | | 2.55 (m, 2H), 1.14 (t, J = 7.6 Hz, 3H). |
| Syn. Ex. | 524.07 (M + 1)/ | δ 9.78 (br s, 1H), 9.03 (br t, J = 5.3 Hz, 1H), 7.74 (s, |
| 19 | 523.15 for | 1H), 7.54 (s, 1H), 7.38 (s, 1H), 6.99 (br s, 1H), 6.85 (br |
| | C25H25N5O6S | s, 1H), 6.74 (br s, 1H), 5.37 (s, 2H), 4.24-4.04 (m, 3H), |
| | | 3.86 (s, 3H), 3.74 (s, 3H), 2.25 (s, 3H), 2.18 (s, 3H). |
| Syn. Ex. | 550.03 (M + 1)/ | δ 9.79 (br s, 1H), 7.80 (s, 1H), 7.54 (s, 1H), 7.45 (s, 1H), |
| 20 | 549.14 for | 7.27 (t, J = 5.9 Hz, 1H), 6.99 (br s, 1H), 6.84 (br s, 1H), |
| | C23H27N5O7S2 | 6.75 (br s, 1H), 5.39 (s, 2H), 4.01 (d, J = 5.9 Hz, 2H), |
| | | 3.86 (s, 3H), 3.74 (s, 3H), 2.81 (s, 3H), 2.25 (s, 3H), |
| | | 2.18 (s, 4H). |
| Syn. Ex. | 544.31(M + 1)/ | δ 11.03-10.28 (m, 1H), 9.03 (br t, J = 5.8 Hz, 1H), 8.26- |
| 21 | 543.10 for | 7.51 (m, 2H), 7.38 (s, 1H), 7.25 (br s, 1H), 6.88 (br s, |
| | C24H22ClN5O6S | 1H), 6.75 (s, 1H), 5.38 (s, 2H), 4.20-4.05 (m, 3H), 3.83 |
| | | (s, 3H), 3.77 (s, 3H), 2.38 (s, 3H). |
| Syn. Ex. | 569.98 (M + 1)/ | δ 10.47 (br s, 1H), 7.80 (s, 1H), 7.71 (s, 1H), 7.46 (s, |
| 22 | 569.08 for | 1H), 7.33-7.21 (m, 2H), 6.87 (br s, 1H), 6.75 (s, 1H), |
| | C22H24ClN5O7S2 | 5.40 (s, 2H), 4.02 (d, J = 6.0 Hz, 2H), 3.83 (s, 3H), 3.77 |
| | | (s, 3H), 2.82 (s, 3H), 2.38 (s, 3H). |
| Syn. Ex. | 536.33 (M + 1)/ | δ 10.27-9.47 (m, 1H), 9.03 (br t, J = 5.4 Hz, 1H), 7.74 |
| 23 | 535.15 for | (s, 1H), 7.63 (s, 1H), 7.38 (s, 1H), 7.07 (s, 1H), 6.86 (s, |
| | C26H25N5O6S | 1H), 6.75 (s, 1H), 5.37 (s, 2H), 4.18-4.04 (m, 3H), 3.87 |
| | | (s, 3H), 3.76 (s, 3H), 2.98-2.77 (m, 4H), 2.12-1.96 |
| | | (m, 2H) |
| Syn. Ex. | 562.00(M + 1)/ | δ 9.76 (br s, 1H), 7.80 (s, 1H), 7.63 (s, 1H), 7.45 (s, 1H), |
| 24 | 561.14 for | 7.27 (t, J = 6.0 Hz, 1H), 7.06 (br s, 1H), 6.89-6.72 (m, |
| | C24H27N5O7S2 | 2H), 5.39 (s, 2H), 4.01 (d, J = 6.0 Hz, 2H), 3.86 (s, 3H), |
| | | 3.75 (s, 3H), 2.95-2.76 (m, 7H), 2.02 (quin, J = 7.4 Hz, |
| | | 2H). |
| Syn. Ex. | 542.2 (M + 1)/ | δ 9.75 (br s, 1H), 7.71 (s, 1H), 7.63 (s, 1H), 7.42 (br s, |
| 25 | 541.16 for | 1H), 7.37 (s, 1H), 7.06 (br s, 1H), 6.83 (br s, 1H), 6.74 |
| | C25H27N5O7S | (br s, 1H), 5.36 (s, 2H), 4.01 (d, J = 5.9 Hz, 2H), 3.86 |
| | | (s, 3H), 3.75 (s, 3H), 3.51 (s, 3H), 3.08-2.76 (m, 4H), |
| | | 2.20-1.94 (m, 2H). |
| Syn. Ex. | 533.22 (M + 1)/ | δ 10.89 (br s, 1H), 7.94 (d, J = 7.9 Hz, 1H), 7.77 (s, 1H), |
| 26 | 532.12 for | 7.58 (br d, J = 7.9 Hz, 1H), 7.36 (s, 1H), 6.97 (d, J = |
| | C25H20N6O6S | 12.7 Hz, 1H), 6.79 (s, 1H), 5.40 (br d, J = 9.6 Hz, 2H), |
| | | 4.88-4.78 (m, 1H), 4.65 (s, 1H), 4.57 (d, J = 4.4 Hz, |
| | | 1H), 4.41 (br d, J = 19.7 Hz, 2H), 3.85 (s, 3H), 3.80 (s, |
| | | 3H). |
| Syn. Ex. | 538.10 (M + 1)/ | δ 9.78 (s, 1H), 8.85-8.68 (t, 1H), 7.72 (s, 1H), 7.54 (s, |
| 27 | 537.17 for | 1H), 7.36 (s, 1H), 7.00 (s, 1H), 6.84 (s, 1H), 6.79-6.69 |
| | C26H27N5O6S | (s, 1H), 5.39-5.38 (s, 2H), 4.09 (d, J = 5.9 Hz, 2H), |
| | | 3.86 (s, 3H), 3.74 (s, 3H), 2.25 (s, 3H), 2.22-2.15 (m, |
| | | 3H), 1.93-1.93 (s, 3H). |
| Syn. Ex. | 544.30 (M + 1)/ | δ 9.78 (br s, 1H), 8.86 (br s, 1H), 7.74 (s, 1H), 7.54 (s, |
| 28 | 543.16 for | 1H), 7.39 (s, 1H), 6.97 (br s, 1H), 6.83 (br s, 1H), 6.71 |
| | C25H26FN5O6S | (br s, 1H), 5.45 (s, 1H), 5.36 (s, 2H), 5.27-5.20 (m, |
| | | 1H), 4.19 (d, J = 5.9 Hz, 2H), 3.85 (s, 3H), 3.73 (s, 3H), |
| | | 2.25 (s, 3H), 2.18 (s, 3H). |
| Syn. Ex. | 550.11 (M + 1)/ | δ 9.75 (s, 1H), 8.77 (br t, J = 5.4 Hz, 1H), 7.72 (s, 1H), |
| 29 | 549.17 for | 7.63 (s, 1H), 7.36 (s, 1H), 7.07 (s, 1H), 6.84 (s, 1H), |
| | C27H27N5O6S | 6.75 (s, 1H), 5.37 (s, 2H), 4.09 (d, J = 5.8 Hz, 2H), 3.87 |
| | | (s, 3H), 3.76 (s, 3H), 2.96-2.77 (m, 4H), 2.12-2.00 |
| | | (m, 2H), 1.93 (s, 3H). |
| Syn. Ex. | 556.08 (M + 1)/ | δ 9.75 (s, 1H), 8.86 (t, J = 5.3 Hz, 1H), 7.75 (s, 1H), |
| 30 | 555.16 for | 7.63 (s, 1H), 7.39 (s, 1H), 7.07 (s, 1H), 6.86 (s, 1H), |
| | C26H26FN5O6S | 6.74 (s, 1H), 5.62-5.41 (m, 1H), 5.37 (s, 2H), 5.23 (dd, |
| | | J = 3.4, 15.8 Hz, 1H), 4.19 (d, J = 6.0 Hz, 2H), 3.86 (s, |
| | | 3H), 3.76 (s, 3H), 2.90-2.77 (m, 4H), 2.02 (t, J = 7.4 |
| | | Hz, 2H). |
| Syn. Ex. | 659.33 (M + 1)/ | δ 10.00 (s, 1H), 7.71 (s, 1H), 7.62 (d, J = 2.3 Hz, 1H), |
| 31 | 658.24 for | 7.49-7.40 (m, 2H), 7.37 (s, 1H), 7.10 (br d, J = 7.8 Hz, |
| | C30H38N6O9S | 1H), 6.85 (br s, 2H), 6.74 (br s, 1H), 5.37 (s, 2H), 4.01 |
| | | (d, J = 5.9 Hz, 2H), 3.91 (br t, J = 5.9 Hz, 2H), 3.82 (s, |
| | | 3H), 3.74 (s, 3H), 3.10 (br d, J = 5.5 Hz, 2H), 2.59 (q, |
| | | J = 7.6 Hz, 2H), 1.36 (s, 9H), 1.14 (t, J = 7.6 Hz, 3H). |
| Syn. Ex. | 559.85 (M + 1)/ | δ 10.04 (s, 1H), 7.94 (br s, 3H), 7.74 (s, 1H), 7.62 (d, |
| 32 | 558.19 for | J = 2.3 Hz, 1H), 7.50-7.43 (m, 2H), 7.40 (s, 1H), 7.12 |
| | C25H30N6O7S | (d, J = 8.5 Hz, 1H), 6.85 (s, 1H), 6.78 (s, 1H), 5.38 (s, |
| | | 2H), 4.12 (br t, J = 5.5 Hz, 2H), 4.08-4.01 (d, 2H), 3.83 |
| | | (s, 3H), 3.75 (s, 3H), 3.09-2.95 (m, 2H), 2.60 (q, J = |
| | | 7.6 Hz, 2H), 1.14 (t, J = 7.6 Hz, 3H). |
| Syn. Ex. | 578.45 (M + 1)/ | δ 10.09 (s, 1H), 7.90-7.74 (m, 2H), 7.64 (br d, J = 8.3 |
| 33 | 577.17for | Hz, 1H), 7.45 (s, 1H), 7.27 (t, J = 5.9 Hz, 1H), 7.11 (br |
| | C25H31N5O7S2 | d, J = 8.8 Hz, 1H), 6.87 (s, 1H), 6.74 (s, 1H), 5.39 (s, |
| | | 2H), 4.01 (d, J = 6.0 Hz, 2H), 3.80 (s, 3H), 3.73 (s, 3H), |
| | | 2.82 (s, 3H), 1.25 (s, 9H). |
| Syn. Ex. | 560.35(M + 1)/ | δ 10.00 (s, 1H), 7.71 (s, 1H), 7.62 (d, J = 2.1 Hz, 1H), |
| 34 | 559.174 for | 7.50-7.40 (m, 2H), 7.37 (s, 1H), 7.11 (br d, J = 8.4 Hz, |
| | C25H29N5O8S | 1H), 6.86 (s, 1H), 6.74 (s, 1H), 5.37 (s, 2H), 4.68 (br t, |
| | | J = 4.9 Hz, 1H), 4.01 (d, J = 5.9 Hz, 2H), 3.95 (br t, J = |
| | | 5.1 Hz, 2H), 3.82 (s, 3H), 3.74 (s, 3H), 3.52 (q, J = 5.1 |
| | | Hz, 2H), 2.60 (q, J = 7.6 Hz, 2H), 1.14 (t, J = 7.6 Hz, |
| | | 3H). |
| Syn. Ex. | 560.31 (M + 1)/ | δ 10.01 (s, 1H), 8.75 (br t, J = 5.7 Hz, 1H), 7.75 (s, 1H), |
| 35 | 559.12 for | 7.62 (d, J = 2.3 Hz, 1H), 7.52-7.42 (m, 1H), 7.40 (s, |
| | C25H26ClN5O6S | 1H), 7.11 (d, J = 8.5 Hz, 1H), 6.88 (s, 1H), 6.73 (s, 1H), |
| | | 6.31 (d, J = 1.9 Hz, 1H), 5.90 (d, J = 1.9 Hz, 1H), 5.37 |
| | | (s, 2H), 4.18 (d, J = 5.9 Hz, 2H), 3.81 (s, 3H), 3.74 (s, |
| | | 3H), 2.60 (q, J = 7.5 Hz, 2H), 1.14 (t, J = 7.6 Hz, 3H). |
| Syn. Ex. | 592.34(M + 1)/ | (δ 9.94 (s, 1H), 8.86 (br t, J = 5.4 Hz, 1H), 7.86 (dd, |
| 36 | 591.15for | J = 1.6, 7.9 Hz, 1H), 7.75 (s, 1H), 7.64-7.50 (m, 1H), |
| | C29H26FN5O6S | 7.40 (s, 1H), 7.36 (br s, 2H), 7.25-7.17 (m, 4H), 7.09 |
| | | (br t, J = 6.8 Hz, 1H), 6.82 (br s, 1H), 6.70 (br s, 1H), |
| | | 5.57 (d, J = 3.4 Hz, 1H), 5.45 (d, J = 3.4 Hz, 1H), 5.36 |
| | | (s, 2H), 5.29-5.24 (m, 2H), 4.19 (d, J = 5.9 Hz, 2H), |
| | | 3.65 (s, 3H). |
| Syn. Ex. | 598.39 (M + 1)/ | δ 9.96 (br s, 1H), 7.86 (dd, J = 1.5, 7.9 Hz, 1H), 7.80 (s, |
| 37 | 597.13 for | 1H), 7.59-7.53 (m, 1H), 7.46 (s, 1H), 7.38 (br s, 2H), |
| | C27H27N5O7S2 | 7.28 (br t, J = 5.9 Hz, 1H), 7.24-7.19 (m, 4H), 7.08 (br |
| | | s, 1H), 6.86-6.69 (m, 2H), 5.38 (s, 2H), 5.26 (br s, 2H), |
| | | 4.02 (d, J = 5.9 Hz, 2H), 3.67 (br s, 3H), 2.81 (s, 3H). |
| Syn. Ex. | 564.36 (M + 1)/ | δ 10.04 (s, 1H), 7.80 (s, 1H), 7.64 (d, J = 2.3 Hz, 1H), |
| 38 | 563.15 for | 7.50 (br d, J = 7.1 Hz, 1H), 7.45 (s, 1H), 7.27 (t, J = 5.9 |
| | C24H29N5O7S2 | Hz, 1H), 7.11 (br d, J = 8.4 Hz, 1H), 6.87 (br s, 1H), |
| | | 6.75 (br s, 1H), 5.39 (s, 2H), 4.01 (d, J = 6.0 Hz, 2H), |
| | | 3.81 (s, 3H), 3.73 (s, 3H), 2.98-2.88 (m, 1H), 2.82 (s, |
| | | 3H), 1.16 (d, J = 6.9 Hz, 6H). |
| Syn. Ex. | 558.20 (M + 1)/ | δ 10.04 (br s, 1H), 7.86 (s, 1H), 7.62 (d, J = 2.1 Hz, 1H), |
| 39 | 557.17 for | 7.45 (s, 2H), 7.09 (br d, J = 7.3 Hz, 1H), 6.86 (br s, 1H), |
| | C26H28FN5O6S | 6.70 (br s, 1H), 5.39 (s, 2H), 5.24 (d, J = 3.9 Hz, 1H), |
| | | 5.15 (d, J = 4.0 Hz, 1H), 4.37 (br s, 2H), 3.81 (s, 3H), |
| | | 3.73 (s, 3H), 3.01-2.79 (m, 3H), 2.59 (q, J = 7.5 Hz, |
| | | 2H), 1.14 (t, J = 7.6 Hz, 3H). |
| Syn. Ex. | 516.58 (M + 1)/ | δ 10.13 (s, 1H), 8.87 (s, 1H), 7.80 (dd, J = 1.7, 7.9 Hz, |
| 40 | 515.12 for | 1H), 7.75 (s, 1H), 7.67-7.59 (m, 1H), 7.39 (s, 1H), 7.19 |
| | C23H22FN5O6S | (d, J = 7.8 Hz, 1H), 7.12-7.06 (m, 1H), 6.87 (s, 1H), |
| | | 6.74 (s, 1H), 5.60-5.43 (m, 1H), 5.37 (s, 2H), 5.23 (dd, |
| | | J = 3.4, 15.7 Hz, 1H), 4.19 (d, J = 5.9 Hz, 2H), 3.82 (s, |
| | | 3H), 3.78 (s, 3H). |
| Syn. Ex. | 546.63 (M + 1)/ | δ 10.19 (s, 1H), 8.87 (br t, J = 6.5 Hz, 1H), 7.75 (s, 1H), |
| 41 | 545.13 for | 7.39 (s, 1H), 7.30 (d, J = 3.1 Hz, 1H), 7.25-7.11 (m, |
| | C24H24FN5O7S | 2H), 6.88 (s, 1H), 6.75 (s, 1H), 5.71-5.42 (m, 1H), 5.38 |
| | | (s, 2H), 5.23 (dd, J = 3.4, 15.7 Hz, 1H), 4.19 (d, J = 5.9 |
| | | Hz, 2H), 3.82 (s, 3H), 3.73 (d, J = 8.9 Hz, 6H). |
| Syn. Ex. | 526.52(M + 1)/ | δ 10.49-9.67 (m, 1H), 8.35 (br t, J = 5.3 Hz, 1H), 7.75 |
| 42 | 525.16 for | (s, 1H), 7.62 (d, J = 2.4 Hz, 1H), 7.50-7.44 (m, 1H), |
| | C25H27N5O6S | 7.40 (s, 1H), 7.14-7.08 (m, 1H), 6.87 (br d, J = 1.8 Hz, |
| | | 1H), 6.75 (br s, 1H), 6.25-6.17 (m, 1H), 6.10 (d, J = |
| | | 2.4 Hz, 1H), 6.06 (d, J = 2.4 Hz, 1H), 5.37 (s, 2H), 4.17 |
| | | (d, J = 5.6 Hz, 2H), 3.82 (s, 3H), 3.74 (s, 3H), 2.59 (q, |
| | | J = 7.5 Hz, 2H), 1.14 (t, J = 7.6 Hz, 3H). |
| Syn. Ex. | 569.80(M + 1)/ | δ 10.48 (br s, 1H), 8.87 (br t, J = 5.4 Hz, 1H), 7.74 (s, |
| 43 | 568.58 for | 1H), 7.69 (d, J = 2.1 Hz, 1H), 7.54 (br dd, J = 1.3, 5.9 |
| | C26H25FN6O6S | Hz, 1H), 7.39 (s, 1H), 7.29 (br d, J = 5.0 Hz, 1H), 6.86 |
| | | (br s, 1H), 6.73 (br s, 1H), 5.62-5.42 (m, 1H), 5.37 (s, |
| | | 2H), 5.29-5.15 (m, 3H), 4.18 (d, J = 5.9 Hz, 2H), 3.81 |
| | | (s, 3H), 2.74-2.59 (m, 2H), 1.16 (t, J = 7.6 Hz, 3H). |
| Syn. Ex. | 555.39 (M + 1)/ | δ 10.48 (br s, 1H), 7.88-7.65 (m, 2H), 7.61-7.54 (m, |
| 44 | 554.58 for | 1H), 7.43 (br s, 1H), 7.37 (s, 1H), 7.29 (br d, J = 7.5 Hz, |
| | C25H26N6O7S | 1H), 6.84 (br s, 1H), 6.73 (br s, 1H), 5.36 (s, 2H), 5.23 |
| | | (s, 2H), 4.01 (d, J = 5.9 Hz, 2H), 3.82 (s, 3H), 3.51 (s, |
| | | 3H), 2.66-2.60 (m, 2H), 1.16 (t, J = 7.6 Hz, 3H). |
| Syn. Ex. | 587.37 (M + 1)/ | δ 10.78-10.40 (m, 1H), 8.86 (br t, J = 5.4 Hz, 1H), 7.74 |
| 45 | 586.60 for | (s, 1H), 7.63 (d, J = 2.1 Hz, 1H), 7.59-7.51 (m, 1H), |
| | C26H27FN6O7S | 7.48 (br d, J = 7.3 Hz, 1H), 7.39 (s, 1H), 7.33-7.24 (m, |
| | | 1H), 7.13-7.07 (m, 1H), 6.93-6.85 (m, 1H), 6.77- |
| | | 6.67 (m, 1H), 5.57 (d, J = 3.4 Hz, 1H), 5.45 (d, J = 3.4 |
| | | Hz, 1H), 5.35 (br s, 2H), 4.55-4.48 (m, 2H), 4.18 (d, |
| | | J = 5.9 Hz, 2H), 3.78 (s, 3H), 2.59 (q, J = 7.8 Hz, 2H), |
| | | 1.14 (t, J = 7.6 Hz, 3H). |
| Syn. Ex. | 540.65 (M + 1)/ | δ 9.77 (br s, 1H), 8.77 (br t, J = 5.8 Hz, 1H), 8.06-7.54 |
| 46 | 539.56 for | (m, 2H), 7.36 (s, 1H), 7.01-6.38 (m, 4H), 5.36 (s, 2H), |
| | C25H25N5O7S | 4.09 (d, J = 5.8 Hz, 2H), 3.87 (s, 3H), 3.82 (s, 3H), 3.77 |
| | | (s, 3H), 1.93 (s, 3H). |
| Syn. Ex. | 546.29 (M + 1)/ | δ 10.01 (s, 1H), 8.46 (br t, J = 5.4 Hz, 1H), 7.72 (s, 1H), |
| 47 | 545.59 for | 7.62 (d, J = 2.3 Hz, 1H), 7.45 (br d, J = 6.8 Hz, 1H), |
| | C25H28FN5O6S | 7.38 (s, 1H), 7.10 (br d, J = 8.3 Hz, 1H), 6.85 (br s, 1H), |
| | | 6.74 (s, 1H), 5.37 (s, 2H), 5.04 (q, J = 6.7 Hz, 1H), 4.13 |
| | | (d, J = 5.9 Hz, 2H), 3.82 (s, 3H), 3.74 (s, 3H), 2.59 (q, |
| | | J = 7.6 Hz, 2H), 1.43 (d, J = 6.6 Hz, 3H), 1.14 (t, J = 7.6 |
| | | Hz, 3H). |
| Syn. Ex. | 546.29 (M + 1)/ | δ 10.01 (s, 1H), 8.46 (br t, J = 5.4 Hz, 1H), 7.72 (s, 1H), |
| 47a | 545.59 for | 7.62 (d, J = 2.3 Hz, 1H), 7.45 (br d, J = 6.8 Hz, 1H), |
| | C25H28FN5O6S | 7.38 (s, 1H), 7.10 (br d, J = 8.3 Hz, 1H), 6.85 (br s, 1H), |
| | | 6.74 (s, 1H), 5.37 (s, 2H), 5.04 (q, J = 6.7 Hz, 1H), 4.13 |
| | | (d, J = 5.9 Hz, 2H), 3.82 (s, 3H), 3.74 (s, 3H), 2.59 (q, |
| | | J = 7.6 Hz, 2H), 1.43 (d, J = 6.6 Hz, 3H), 1.13 (t, J = |
| | | 7.6 Hz, 3H). |
| Syn. Ex. | 546.29 (M + 1)/ | δ 10.01 (s, 1H), 8.46 (br t, J = 5.4 Hz, 1H), 7.72 (s, 1H), |
| 47b | 545.59 for | 7.62 (d, J = 2.3 Hz, 1H), 7.45 (br d, J = 6.8 Hz, 1H), |
| | C25H28FN5O6S | 7.38 (s, 1H), 7.10 (br d, J = 8.3 Hz, 1H), 6.85 (br s, 1H), |
| | | 6.74 (s, 1H), 5.37 (s, 2H), 5.04 (q, J = 6.7 Hz, 1H), 4.13 |
| | | (d, J = 5.9 Hz, 2H), 3.82 (s, 3H), 3.74 (s, 3H), 2.59 (q, |
| | | J = 7.6 Hz, 2H), 1.43 (d, J = 6.6 Hz, 3H), 1.13 (t, J = |
| | | 7.6 Hz, 3H). |
| Syn. Ex. | 601.83 (M + 1)/ | δ 10.02 (s, 1H), 7.91 (br s, 1H), 7.71 (s, 1H), 7.62 (d, |
| 48 | 600.20 for | J = 2.2 Hz, 1H), 7.46 (br d, J = 6.7 Hz, 2H), 7.38 (s, 1H), |
| | C27H32N6O8S | 7.11 (d, J = 8.6 Hz, 1H), 6.86 (s, 1H), 6.75 (s, 1H), 5.37 |
| | | (s, 2H), 4.01 (d, J = 5.9 Hz, 2H), 3.93 (t, J = 5.9 Hz, |
| | | 2H), 3.82 (s, 3H), 3.75 (s, 3H), 3.21 (q, J = 5.7 Hz, 2H), |
| | | 2.60 (q, J = 7.6 Hz, 2H), 1.78 (s, 3H), 1.14 (t, J = 7.6 |
| | | Hz, 3H). |
| Syn. Ex. | 554.37(M + 1)/ | δ 10.03 (br s, 1H), 8.92 (br t, J = 5.7 Hz, 1H), 7.73 (s, |
| 49 | 553.59 for | 1H), 7.62 (d, J = 2.2 Hz, 1H), 7.53-7.43 (m, 1H), 7.37 |
| | C26H27N5O7S | (s, 1H), 7.10 (br d, J = 8.3 Hz, 1H), 6.86 (s, 1H), 6.74 |
| | | (s, 1H), 5.37 (s, 2H), 4.19 (s, 2H), 4.11 (d, J = 5.9 Hz, |
| | | 3H), 3.82 (s, 3H), 3.74 (s, 3H), 2.60 (q, J = 7.5 Hz, 2H), |
| | | 1.14 (t, J = 7.5 Hz, 3H). |
| Syn. Ex. | 568.17 (M + 1)/ | δ 10.85-9.97 (m, 1H), 7.74 (s, 1H), 7.54-7.38 (m, |
| 50 | 567.12 for | 3H), 7.32-7.03 (m, 3H), 6.77 (d, J = 8.5 Hz, 2H), 5.44 |
| | C23H23F2N5O8S | (s, 2H), 4.02 (d, J = 5.9 Hz, 2H), 3.75 (s, 6H), 3.52 (s, |
| | | 3H). |
| Syn. Ex. | 557.2 (M + 1)/ | δ 10.05 (s, 1H), 9.10 (br t, J = 5.8 Hz, 1H), 8.10 (d, J = |
| 51 | 556.14 for | 2.0 Hz, 1H), 7.80 (dd, J = 2.4, 8.4 Hz, 1H), 7.64 (d, J = |
| | C26H25FN4O7S | 2.3 Hz, 1H), 7.54-7.45 (m, 1H), 7.13 (br d, J = 8.1 Hz, |
| | | 1H), 7.08 (d, J = 8.4 Hz, 1H), 6.91 (br s, 1H), 6.65 (br |
| | | s, 1H), 5.64-5.45 (m, 1H), 5.28 (dd, J = 3.4, 15.7 Hz, |
| | | 1H), 4.34 (d, J = 6.0 Hz, 2H), 3.81 (s, 3H), 3.78 (s, 3H), |
| | | 2.64-2.57 (m, 2H), 1.15 (t, J = 7.6 Hz, 3H). |
| Syn. Ex. | 580.20(M + 1)/ | δ 11.11-10.19 (m, 1H), 8.86 (br t, J = 5.8 Hz, 1H), 7.77 |
| 52 | 579.14 for | (s, 1H), 7.60 (d, J = 2.1 Hz, 1H), 7.46 (br d, J = 7.6 Hz, |
| | C25H24F3N5O6S | 1H), 7.41 (s, 1H), 7.25 (br s, 2H), 7.11 (br d, J = 8.4 Hz, |
| | | 1H), 7.01 (br s, 1H), 5.61-5.45 (m, 1H), 5.43 (s, 2H), |
| | | 5.23 (dd, J = 3.4, 15.6 Hz, 1H), 4.19 (d, J = 5.9 Hz, 2H), |
| | | 3.71 (s, 3H), 2.59 (q, J = 7.5 Hz, 2H), 1.14 (t, J = 7.6 |
| | | Hz, 3H). |
| Syn. Ex. | 560.22(M + 1)/ | δ 11.11-10.38 (m, 1H), 9.04 (br t, J = 5.6 Hz, 1H), |
| 53 | 559.13 for | 7.76 (s, 1H), 7.60 (d, J = 2.2 Hz, 1H), 7.50-7.42 (m, |
| | C25H23F2N5O6S | 1H), 7.40 (s, 1H), 7.32-7.26 (m, 1H), 7.23 (br s, 1H), |
| | | 7.11 (br d, J = 8.6 Hz, 1H), 7.02 (br s, 1H), 5.43 (s, 2H), |
| | | 4.28-4.06 (m, 3H), 3.71 (s, 3H), 2.59 (q, J = 7.5 Hz, |
| | | 2H), 1.14 (t, J = 7.6 Hz, 3H). |
| Syn. Ex. | 602.20 (M + 1)/ | δ 9.59 (s, 1H), 9.26-8.68 (m, 1H), 7.74 (s, 1H), 7.67 |
| 54 | 601.16 for | (d, J = 2.1 Hz, 1H), 7.54-7.42 (m, 1H), 7.38 (s, 1H), |
| | C27H28FN5O8S | 7.09-7.01 (m, 1H), 6.88 (br s, 1H), 6.73 (br s, 1H), |
| | | 5.59-5.42 (m, 1H), 5.36 (s, 2H), 5.23 (dd, J = 3.4, 15.7 |
| | | Hz, 1H), 4.90 (br s, 2H), 4.18 (d, J = 5.9 Hz, 2H), 3.84 |
| | | (s, 3H), 3.52 (s, 3H), 2.64-2.58 (m, 2H), 1.15 (t, J = |
| | | 7.6 Hz, 3H). |
| Syn. Ex. | 588.20 (M + 1)/ | δ = 9.59 (s, 1H), 7.70 (s, 1H), 7.67 (d, J = 2.1 Hz, 1H), |
| 55 | 587.17 for | 7.42 (br s, 2H), 7.36 (s, 1H), 7.10-7.02 (m, 1H), 6.86 |
| | C26H29N5O9S | (br s, 1H), 6.74 (br s, 1H), 5.36 (s, 2H), 4.90 (br s, 2H), |
| | | 4.01 (d, J = 5.9 Hz, 2H), 3.85 (s, 3H), 3.51 (s, 6H), 2.64- |
| | | 2.58 (m, 2H), 1.15 (t, J = 7.5 Hz, 3H). |
| Syn. Ex. | 543.20 (M + 1)/ | δ 10.05 (br s, 1H), 8.07 (d, J = 1.9 Hz, 1H), 7.77 (dd, |
| 56 | 542.15 for | J = 2.3, 8.4 Hz, 1H), 7.72-7.67 (m, 1H), 7.65 (d, J = 2.0 |
| | C25H26N4O8S | Hz, 1H), 7.46 (br d, J = 7.6 Hz, 1H), 7.12 (br d, J = 8.1 |
| | | Hz, 1H), 7.07 (d, J = 8.4 Hz, 1H), 6.89 (br s, 1H), 6.63 |
| | | (br s, 1H), 4.16 (br d, J = 6.0 Hz, 2H), 3.82 (s, 3H), 3.78 |
| | | (s, 3H), 3.54 (s, 3H), 2.61 (q, J = 7.5 Hz, 2H), 1.15 (t, |
| | | J = 7.6 Hz, 3H) |
| Syn. Ex. | 551.2(M + 1)/ | δ 10.05 (s, 1H), 9.01 (t, J = 6.0 Hz, 1H), 8.06 (d, J = |
| 57 | 550.15 for | 2.0 Hz, 1H), 7.75 (dd, J = 2.4, 8.4 Hz, 1H), 7.65 (d, J = |
| | C27H26N4O7S | 2.3 Hz, 1H), 7.47 (br d, J = 6.1 Hz, 1H), 7.13 (br d, J = |
| | | 7.6 Hz, 1H), 7.07 (d, J = 8.4 Hz, 1H), 6.90 (br s, 1H), |
| | | 6.64 (br s, 1H), 4.24 (d, J = 6.0 Hz, 2H), 3.81 (s, 3H), |
| | | 3.78 (s, 3H), 2.61 (q, J = 7.5 Hz, 2H), 1.95 (s, 3H), 1.15 |
| | | (t, J = 7.6 Hz, 3H). |
| Syn. Ex. | 540.53(M + 1)/ | δ 10.18 (s, 1H), 8.78 (br t, J = 5.6 Hz, 1H), 7.72 (s, 1H), |
| 58 | 539.15 for | 7.37 (s, 1H), 7.30 (d, J = 3.2 Hz, 1H), 7.22-7.17 (m, |
| | C25H25N5O7S | 1H), 7.16-7.11 (m, 1H), 6.86 (br s, 1H), 6.74 (s, 1H), |
| | | 5.37 (s, 2H), 4.09 (d, J = 5.7 Hz, 2H), 3.83 (s, 3H), 3.74 |
| | | (s, 3H), 3.72 (s, 3H), 1.93 (s, 3H). |
| Syn. Ex. | 558.72(M + 1)/ | δ 10.03 (s, 1H), 8.86 (br t, J = 5.8 Hz, 1H), 7.74 (s, 1H), |
| 59 | 557.17 for | 7.64 (d, J = 2.3 Hz, 1H), 7.54-7.46 (m, 1H), 7.39 (s, |
| | C26H28FN5O6S | 1H), 7.11 (br d, J = 8.5 Hz, 1H), 6.88 (s, 1H), 6.73 (s, |
| | | 1H), 5.62-5.41 (m, 1H), 5.37 (s, 2H), 5.23 (dd, J = 3.4, |
| | | 15.8 Hz, 1H), 4.19 (d, J = 5.9 Hz, 2H), 3.80 (s, 3H), |
| | | 3.73 (s, 3H), 2.95-2.86 (m, 1H), 1.17 (s, 3H), 1.15 (s, |
| | | 3H). |
| Syn. Ex. | 546.58(M + 1)/ | δ 9.77 (s, 1H), 8.86 (br t, J = 5.6 Hz, 1H), 8.10-7.51 |
| 60 | 545.14for | (m, 2H), 7.39 (s, 1H), 6.86 (s, 1H), 6.74 (s, 1H), 6.68- |
| | C24H24FN5O7S | 6.59 (m, 2H), 5.62-5.43 (m, 1H), 5.37 (s, 2H), 5.23 |
| | | (dd, J = 3.4, 15.6 Hz, 1H), 4.19 (d, J = 5.9 Hz, 2H), 3.86 |
| | | (s, 3H), 3.82 (s, 3H), 3.78 (s, 3H). |
| Syn. Ex. | 537.20 (M + 1)/ | δ 10.05 (br s, 1H), 9.26 (br t, J = 5.8 Hz, 1H), 8.07 (d, |
| 61 | 536.14 for | J = 2.1 Hz, 1H), 7.76 (dd, J = 2.3, 8.4 Hz, 1H), 7.65 (d, |
| | C26H24N4O7S | J = 2.1 Hz, 1H), 7.54-7.42 (m, 1H), 7.19-7.03 (m, |
| | | 2H), 6.91 (br s, 1H), 6.64 (br s, 1H), 4.27 (d, J = 6.0 Hz, |
| | | 2H), 4.18 (s, 1H), 3.81 (s, 3H), 3.77 (s, 3H), 2.61 (q, |
| | | J = 7.4 Hz, 2H), 1.15 (t, J = 7.6 Hz, 3H). |
| Syn. Ex. | 574.20 (M + 1)/ | δ 7.71 (s, 1H), 7.64 (d, J = 2.0 Hz, 1H), 7.43 (br dd, J = |
| 62 | 573.15 for | 1.9, 8.2 Hz, 2H), 7.37 (s, 1H), 7.06 (d, J = 8.5 Hz, 1H), |
| | C25H27N5O9S | 6.82 (s, 1H), 6.72 (s, 1H), 5.36 (s, 2H), 4.75 (s, 2H), |
| | | 4.01 (d, J = 5.9 Hz, 2H), 3.84 (s, 3H), 3.51 (s, 3H), 2.64- |
| | | 2.57 (m, 2H), 1.15 (t, J = 7.5 Hz, 3H) exchangeable |
| | | protons not observed. |
| Syn. Ex. | 564.30 (M − 1)/ | δ 10.73-10.51 (m, 1H), 7.73 (s, 1H), 7.60 (d, J = 2.1 |
| 63 | 565.14 for | Hz, 1H), 7.44 (br d, J = 4.8 Hz, 2H), 7.38 (s, 1H), 7.22 |
| | C24H25F2N5O7S | (br s, 1H), 7.10 (br d, J = 7.3 Hz, 1H), 7.01 (br d, J = |
| | | 1.1 Hz, 1H), 5.43 (s, 2H), 4.02 (d, J = 5.9 Hz, 2H), 3.71 |
| | | (s, 3H), 3.52 (s, 3H), 2.59 (q, J = 7.6 Hz, 2H), 1.14 (t, |
| | | J = 7.6 Hz, 3H). |
| Syn. Ex. | 560.20 (M + 1)/ | δ 9.89-9.80 (m, 1H), 7.71 (s, 1H), 7.63 (d, J = 2.3 Hz, |
| 64 | 559.17 for | 1H), 7.43 (br s, 2H), 7.36 (s, 1H), 7.15 (br d, J = 3.0 Hz, |
| | C25H29N5O8S | 1H), 6.84 (br s, 1H), 6.72 (br s, 1H), 5.36 (s, 2H), 5.06 |
| | | (m, 1H), 4.07-3.99 (m, 4H), 3.82 (s, 3H), 3.70-3.63 |
| | | (m, 2H), 3.51 (s, 3H), 2.62-2.59 (m, 2H), 1.16 (t, J = |
| | | 7.6 Hz, 3H). |
| Syn. Ex. | 554.32 (M + 1)/ | δ 9.85 (br s, 1H), 9.03 (br t, J = 5.5 Hz, 1H), 7.75 (s, |
| 65 | 553.16 for | 1H), 7.63 (d, J = 2.1 Hz, 1H), 7.45 (br d, J = 4.8 Hz, |
| | C26H27N5O7S | 1H), 7.38 (s, 1H), 7.16 (br d, J = 8.0 Hz, 1H), 6.85 (br |
| | | d, J = 9.6 Hz, 1H), 6.73 (br s, 1H), 5.37 (s, 2H), 5.08 |
| | | (br s, 1H), 4.13-4.09 (m, 2H), 4.05 (t, J = 4.8 Hz, 2H), |
| | | 3.82 (s, 3H), 3.76-3.61 (m, 3H), 2.61 (q, J = 7.5 Hz, |
| | | 2H), 1.16 (t, J = 7.6 Hz, 3H) |
| Syn. Ex. | 643.39 (M + 1)/ | δ 10.12 (s, 1H), 7.72 (d, J = 1.0 Hz, 1H), 7.63 (s, 1H), |
| 66 | 642.21 for | 7.49 (t, J = 1.0 Hz, 1H), 7.41 (br s, 1H), 7.37 (d, J = 1.0 |
| | C29H34N6O9S | Hz, 1H), 7.14 (br d, J = 8.3 Hz, 1H), 6.86 (br s, 1H), |
| | | 6.75 (d, J = 1.0 Hz, 1H), 5.36 (s, 2H), 4.99 (br s, 2H), |
| | | 4.01 (br d, J = 5.8 Hz, 2H), 3.85 (s, 3H), 3.52 (br s, 8H), |
| | | 3.40 (br s, 3H), 2.62 (q, J = 1.0 Hz, 2H), 1.16 (t, J = 7.6 |
| | | Hz, 3H) |
| Syn. Ex. | 588.20 (M + 1)/ | δ 8.85 (br t, J = 5.5 Hz, 1H), 7.71 (s, 1H), 7.55 (d, J = |
| 67 | 587.15 for | 1.4 Hz, 1H), 7.37 (s, 1H), 7.26 (dd, J = 2.2, 8.3 Hz, 1H), |
| | C26H26FN5O8S | 7.20-7.08 (m, 2H), 7.01 (d, J = 8.4 Hz, 1H), 6.71 (s, |
| | | 1H), 6.58 (s, 1H), 5.61-5.42 (m, 1H), 5.32 (s, 2H), 5.22 |
| | | (dd, J = 3.4, 15.8 Hz, 1H), 4.45 (br s, 2H), 4.18 (d, J = |
| | | 5.9 Hz, 2H), 3.76 (br s, 3H), 2.61-2.53 (m, 2H), 1.15 |
| | | (t, J = 7.6 Hz, 3H) |
| Syn. Ex. | 657.30 (M + 1)/ | δ 10.14 (br s, 1H), 8.86 (br s, 1H), 7.74 (s, 1H), 7.63 (br |
| 68 | 656.21 for | s, 1H), 7.49-7.46 (m, 1H), 7.38 (s, 1H), 7.16-7.14 (m, |
| | C30H33FN6O8S | 1H), 6.90-6.71 (m, 2H), 5.58-5.44 (m, 1H), 5.36 (s, |
| | | 2H), 5.23 (dd, J = 3.4, 15.6 Hz, 1H), 5.00-4.98 (m, |
| | | 2H), 4.18 (d, J = 5.9 Hz, 2H), 3.84 (s, 3H), 3.52 (br s, |
| | | 4H), 3.40 (br s, 4H), 2.63-2.60 (m, 2H), 1.16 (t, J = 7.5 |
| | | Hz, 3H) |
| Syn. Ex. | 549.28 (M + 1)/ | δ 10.47 (s, 1H), 9.03 (br s, 1H), 7.73 (s, 1H), 7.69 (br d, |
| 69 | 548.15 for | J = 1.9 Hz, 1H), 7.38 (m, 1H), 7.29-7.22 (m, 1H), 6.87- |
| | C26H24N6O6S | 6.68 (m, 2H), 6.51 (s, 1H), 5.35 (br s, 2H), 5.21 (br s, |
| | | 2H), 4.14-4.08 (m, 2H), 3.82 (s, 3H), 3.69 (br s, 1H), |
| | | 2.69-2.57 (m, 2H), 1.15 (br t, J = 7.5 Hz, 3H) |
| Syn. Ex. | 592.20 (M + 1)/ | δ 9.02 (br t, J = 5.7 Hz, 1H), 7.73 (s, 1H), 7.69 (d, J = |
| 70 | 591.16 for | 2.4 Hz, 1H), 7.50 (dd, J = 2.2, 8.4 Hz, 1H), 7.37 (s, 1H), |
| | C26H25N9O6S | 7.33 (d, J = 8.5 Hz, 1H), 6.83 (s, 1H), 6.70 (s, 1H), 5.54 |
| | | (s, 2H), 5.36 (s, 2H), 4.12-4.10 (m, 3H), 3.79 (s, 3H), |
| | | 2.64 (m, 2H), 1.16 (t, J = 7.6 Hz, 3H) |
| Syn. Ex. | 598.30 (M + 1)/ | δ 7.68 (m, 2H), 7.42 (dd, J = 2.2, 8.3 Hz, 2H), 7.36- |
| 71 | 597.18 for | 7.25 (m, 2H), 6.74 (s, 1H), 6.64 (s, 1H), 5.44 (s, 2H), |
| | C25H27N9O7S | 5.33 (s, 2H), 4.00 (d, J = 5.9 Hz, 2H), 3.83 (s, 3H), 3.51 |
| | | (s, 3H), 2.67-2.60 (m, 2H), 1.18 (t, J = 7.6 Hz, 3H) |
| Syn. Ex. | 612.27 (M + 1)/ | δ 10.12 (br s, 1H), 8.87 (br t, J = 5.4 Hz, 1H), 7.75 (s, |
| 72 | 611.17 for | 1H), 7.69 (d, J = 2.1 Hz, 1H), 7.53 (dd, J = 2.2, 8.4 Hz, |
| | C26H26FN9O6S | 1H), 7.40-7.33 (m, 2H), 6.86 (s, 1H), 6.72 (s, 1H), 5.59- |
| | | 5.43 (m, 3H), 5.36 (s, 2H), 5.23 (dd, J = 3.4, 15.6 Hz, |
| | | 1H), 4.18 (br d, J = 5.9 Hz, 2H), 3.77 (s, 3H), 2.69- |
| | | 2.60 (m, 2H), 1.16 (t, J = 7.6 Hz, 3H) |
| Syn. Ex. | 612.20 (M + 1)/ | δ 9.68 (s, 1H), 9.55 (s, 1H), 8.86 (br t, J = 5.3 Hz, 1H), |
| 73 | 611.16 for | 7.75 (s, 1H), 7.70 (d, J = 2.1 Hz, 1H), 7.51 (br d, J = 8.1 |
| | C27H26FN7O7S | Hz, 1H), 7.39 (s, 1H), 7.29 (br d, J = 8.0 Hz, 1H), 6.86 |
| | | (br s, 1H), 6.71 (s, 1H), 5.60-5.35 (m, 5H), 5.23 (dd, |
| | | J = 3.4, 15.6 Hz, 1H), 4.19 (d, J = 6.0 Hz, 2H), 3.81 (s, |
| | | 3H), 2.63 (br d, J = 7.5 Hz, 2H), 1.16 (t, J = 7.6 Hz, |
| | | 3H). |
| Syn. Ex. | 598.20 (M + 1)/ | δ 9.58 (s, 1H), 8.15 (s, 1H), 7.68 (s, 2H), 7.42 (br s, 1H), |
| 74 | 597.16 for | 7.35 (s, 1H), 7.25-6.96 (m, 2H), 6.66-6.49 (m, 2H), |
| | C26H27N7O8S | 5.33-5.23 (m, 4H), 4.01 (d, J = 5.9 Hz, 2H), 3.80 (s, |
| | | 3H), 3.51 (s, 3H), 2.56 (br s, 2H), 1.13 (t, J = 7.6 Hz, |
| | | 3H). |
| Syn. Ex. | 559.20 (M + 1)/ | δ 8.50-8.00 (m, 2H), 7.68 (s, 1H), 7.65 (d, J = 2.3 Hz, |
| 75 | 558.19 for | 1H), 7.46-7.43 (m, 1H), 7.34 (s, 1H), 7.23 (dd, J = 2.4, |
| | C25H30N6O7S | 8.3 Hz, 1H), 7.00 (d, J = 8.1 Hz, 1H), 6.67 (s, 1H), 6.57 |
| | | (s, 1H), 5.29 (s, 2H), 4.08 (t, J = 4.8 Hz, 2H), 4.00 (d, |
| | | J = 5.9 Hz, 2H), 3.85 (s, 3H), 3.51 (s, 3H), 3.09 (t, J = 4.8 |
| | | Hz, 2H), 2.61 (q, J = 7.6 Hz, 2H), 1.19 (t, J = 7.6 Hz, |
| | | 3H). |
| Syn. Ex. | 573.20 (M + 1)/ | δ 8.86 (br t, J = 5.1 Hz, 1H), 8.32 (br s, 3H), 7.72 (s, |
| 76 | 572.19 for | 1H), 7.65 (d, J = 2.3 Hz, 1H), 7.37 (s, 1H), 7.23 (dd, |
| | C26H29FN6O6S | J = 2.4, 8.3 Hz, 1H), 7.01 (d, J = 8.1 Hz, 1H), 6.68 (s, |
| | | 1H), 6.57 (s, 1H), 5.61-5.41 (m, 1H), 5.30 (s, 2H), 5.23 |
| | | (dd, J = 3.4, 15.8 Hz, 1H), 4.17 (d, J = 6.0 Hz, 2H), 4.08 |
| | | (t, J = 4.9 Hz, 2H), 3.85 (s, 3H), 3.10 (br t, J = 4.8 Hz, |
| | | 2H), 2.61 (q, J = 7.5 Hz, 2H), 1.19 (t, J = 7.6 Hz, 3H). |
| Syn. Ex. | 629 (M + 1)/ | δ 9.74 (s, 1H), 7.71 (s, 1H), 7.67 (d, J = 2.3 Hz, 1H), |
| 77 | 628.20 for | 7.50-7.40 (m, 2H), 7.37 (s, 1H), 7.12 (br d, J = 8.1 Hz, |
| | C28H32N6O9S | 1H), 6.90-6.71 (m, 2H), 5.36 (s, 2H), 4.26-4.18 (m, |
| | | 2H), 4.13 (br s, 2H), 4.01 (d, J = 5.9 Hz, 2H), 3.82 (s, |
| | | 3H), 3.66-3.59 (m, 2H), 3.51 (s, 4H), 2.61 (q, J = 7.5 |
| | | Hz, 3H), 1.15 (t, J = 7.6 Hz, 3H) |
| Syn. Ex. | 643.30 (M + 1)/ | δ 9.74 (s, 1H), 8.86 (br t, J = 5.8 Hz, 1H), 7.75 (s, 1H), |
| 78 | 642.19 for | 7.66 (d, J = 2.3 Hz, 1H), 7.47 (br d, J = 7.8 Hz, 1H), |
| | C29H31FN6O8S | 7.39 (s, 1H), 7.12 (br d, J = 8.3 Hz, 1H), 6.87 (br s, 1H), |
| | | 6.73 (br s, 1H), 5.58-5.43 (m, 1H), 5.37 (s, 2H), 5.23 |
| | | (dd, J = 3.4, 15.6 Hz, 1H), 4.26-4.09 (m, 6H), 3.82 (s, |
| | | 3H), 3.62 (br d, J = 6.0 Hz, 2H), 3.53-3.48 (m, 2H), |
| | | 2.61 (q, J = 7.7 Hz, 2H), 1.15 (t, J = 7.6 Hz, 3H). |
| Syn. Ex. | 631.30 (M + 1)/ | δ 9.96 (br s, 1H), 8.86 (br t, J = 5.3 Hz, 1H), 7.75 (s, |
| 79 | 630.19 for | 1H), 7.66 (d, J = 2.1 Hz, 1H), 7.45 (br d, J = 8.0 Hz, |
| | C28H31FN6O8S | 1H), 7.39 (s, 1H), 7.27 (br t, J = 5.6 Hz, 1H), 7.09 (br |
| | | d, J = 8.6 Hz, 1H), 6.86 (s, 1H), 6.74 (s, 1H), 5.58-5.44 |
| | | (m, 1H), 5.37 (s, 2H), 5.23 (dd, J = 3.4, 15.8 Hz, 1H), |
| | | 4.18 (d, J = 5.9 Hz, 2H), 3.98 (br t, J = 4.9 Hz, 2H), |
| | | 3.84 (s, 3H), 3.51 (s, 3H), 3.37 (br d, J = 5.1 Hz, 2H), |
| | | 2.62 (q, J = 7.4 Hz, 2H), 1.16 (t, J = 1.0 Hz, 3H) |
| Syn. Ex. | 611.24 (M + 1)/ | δ 14.18-14.04 (m., 1H), 10.45 (br s, 1H), 8.85 (br t, J = |
| 80 | 610.18 for | 5.7 Hz, 1H), 8.53-8.45 (m, 1H), 7.74 (s, 1H), 7.65 (d, |
| | C27H27FN8O6S | J = 2.1 Hz, 1H), 7.49 (br d, J = 6.9 Hz, 1H), 7.40-7.33 |
| | | (m, 2H), 6.90-6.65 (m, 2H), 5.58-5.43 (m, 1H), 5.36 |
| | | (s, 2H), 5.28-5.19 (m, 3H), 4.18 (d, J = 5.9 Hz, 2H), |
| | | 3.85 (s, 3H), 2.63 (q, J = 7.7 Hz, 2H), 1.17 (t, J = 7.6 |
| | | Hz, 3H) |
| Syn. Ex. | 601.30 (M + 1)/ | δ 10.00 (s, 1H), 8.03 (br t, J = 5.6 Hz, 1H), 7.71 (s, 1H), |
| 81 | 600.20 for | 7.64 (d, J = 2.3 Hz, 1H), 7.48-7.40 (m, 2H), 7.37 (s, |
| | C27H32N6O8S | 1H), 7.09 (d, J = 8.6 Hz, 1H), 6.85 (s, 1H), 6.74 (s, 1H), |
| | | 5.37 (s, 2H), 4.03-3.97 (m, 4H), 3.82 (s, 3H), 3.51 (s, |
| | | 3H), 3.43 (q, J = 5.3 Hz, 2H), 2.61 (q, J = 7.5 Hz, 2H), |
| | | 1.81 (s, 3H), 1.15 (t, J = 7.6 Hz, 3H) |
| Syn. Ex. | 615.30 (M + 1)/ | δ 10.01 (br s, 1H), 8.86 (br t, J = 5.7 Hz, 1H), 8.16- |
| 82 | 614.20 for | 8.00 (m, 1H), 7.74 (s, 1H), 7.64 (d, J = 1.9 Hz, 1H), |
| | C28H31FN6O7S | 7.39 (s, 2H), 7.08 (br d, J = 4.4 Hz, 1H), 6.72 (br d, J = |
| | | 1.8 Hz, 2H), 5.58-5.44 (m, 1H), 5.36 (br s, 2H), 5.23 |
| | | (dd, J = 3.4, 15.8 Hz, 1H), 4.26-3.95 (m, 2H), 4.03- |
| | | 3.87 (m, 2H), 3.81 (s, 3H), 3.52-3.34 (m, 2H), 2.70- |
| | | 2.57 (m, 2H), 1.81 (s, 3H), 1.15 (t, J = 7.6 Hz, 3H) |
| Syn. Ex. | 569.39 (M + 1)/ | δ 7.71-7.63 (m, 2H), 7.42 (br s, 1H), 7.38-7.34 (m, |
| 83 | 568.17 for | 2H), 7.10 (br s, 1H), 6.76-6.63 (m, 2H), 5.33 (s, 2H), |
| | C26H28N6O7S | 4.19 (br s, 2H), 4.01 (d, J = 5.8 Hz, 2H), 3.82 (s, 3H), |
| | | 3.51 (s, 3H), 2.96 (t, J = 6.3 Hz, 2H), 2.59 (q, J = 7.5 |
| | | Hz, 2H), 1.15 (t, J = 7.6 Hz, 3H) |
| Syn. Ex. | 583.20 (M + 1)/ | δ 9.92 (s, 1H), 8.87 (br t, J = 5.7 Hz, 1H), 7.75 (s, 1H), |
| 84 | 582.17 for | 7.66 (d, J = 2.2 Hz, 1H), 7.48 (br d, J = 7.2 Hz, 1H), |
| | C27H27FN6O6S | 7.39 (s, 1H), 7.20 (br d, J = 8.3 Hz, 1H), 6.87 (s, 1H), |
| | | 6.74 (s, 1H), 5.59-5.44 (m, 1H), 5.37 (s, 2H), 5.23 (dd, |
| | | J = 3.4, 15.7 Hz, 1H), 4.27 (br t, J = 6.0 Hz, 2H), 4.19 |
| | | (d, J = 5.9 Hz, 2H), 3.81 (s, 3H), 3.00 (t, J = 6.1 Hz, |
| | | 2H), 2.62 (q, J = 7.6 Hz, 2H), 1.15 (t, J = 7.6 Hz, 3H) |
| Syn. Ex. | 527.25 (M + 1)/ | δ 9.03 (br t, J = 5.6 Hz, 1H), 8.12 (d, J = 1.0 Hz, 1H), |
| 85 | 526.13 for | 7.74 (s, 1H), 7.38 (s, 1H), 6.79 (d, J = 1.0 Hz, 2H), 6.65 |
| | C23H22N6O7S | (s, 1H), 6.51 (s, 1H), 5.35 (s, 2H), 4.11 (t, J = 1.0 Hz, |
| | | 3H), 3.83 (s, 3H), 3.75 (br s, 6H) |
| Syn. Ex. | 533.20 (M + 1)/ | δ 8.25-8.11 (d, 1H), 7.72 (s, 1H), 7.48-7.35 (d, 2H), |
| 86 | 532.14 for | 6.95-6.83 (t, 2H), 6.75 (s, 1H), 6.46 (s, 1H), 5.38 (s, |
| | C22H24N6O8S | 2H), 4.02 (d, J = 5.9 Hz, 2H), 3.83 (d, J = 1.0 Hz, 9H), |
| | | 3.52 (s, 3H) |
| Syn. Ex. | 547.20 (M + 1)/ | δ 10.18 (s, 1H), 8.86 (br t, J = 5.7 Hz, 1H), 8.22 (br d, |
| 87 | 546.13 for | J = 5.6 Hz, 1H), 7.76 (s, 1H), 7.40 (s, 1H), 6.95-6.86 |
| | C23H23FN6O7S | (m, 2H), 6.75 (s, 1H), 5.59-5.44 (m, 1H), 5.38 (s, 2H), |
| | | 5.23 (dd, J = 3.4, 15.8 Hz, 1H), 4.19 (d, J = 5.9 Hz, 2H), |
| | | 3.82 (s, 9H) |
| Syn. Ex. | 516.25 (M + 1)/ | δ 10.02 (br s, 1H), 7.71 (s, 1H), 7.61 (d, J = 2.0 Hz, 1H), |
| 88 | 515.15 for | 7.48-7.33 (m, 3H), 7.06 (br d, J = 7.0 Hz, 1H), 6.90- |
| | C23H25N5O7S | 6.62 (m, 2H), 5.36 (s, 2H), 4.01 (d, J = 5.9 Hz, 2H), |
| | | 3.83 (s, 3H), 3.73 (s, 3H), 3.51 (s, 3H), 2.28 (s, 3H) |
| Syn. Ex. | 530.20 (M + 1)/ | δ 9.98 (s, 1H), 8.86 (br t, J = 5.8 Hz, 1H), 7.75 (s, 1H), |
| 89 | 529.14 for | 7.61 (d, J = 1.8 Hz, 1H), 7.45-7.36 (m, 2H), 7.09 (d, |
| | C24H24FN5O6S | J = 8.5 Hz, 1H), 6.87 (s, 1H), 6.74 (s, 1H), 5.60-5.44 (m, |
| | | 1H), 5.37 (s, 2H), 5.23 (dd, J = 3.4, 15.8 Hz, 1H), 4.19 |
| | | (d, J = 6.0 Hz, 2H), 3.83 (s, 3H), 3.74 (s, 3H), 2.29 (s, |
| | | 3H) |
| Syn. Ex. | 515.30 (M + 1)/ | δ 10.01 (m, 1H), 7.69 (s, 1H), 7.62 (d, J = 1.9 Hz, 1H), |
| 90 | 514.16 for | 7.47-7.43 (m, 1H), 7.37 (s, 1H), 7.10 (br d, J = 8.3 Hz, |
| | C23H26N6O6S | 1H), 6.85 (br s, 1H), 6.75 (br s, 1H), 6.12 (br t, J = 5.2 |
| | | Hz, 1H), 5.44-5.34 (m, 4H), 3.99 (d, J = 5.6 Hz, 2H), |
| | | 3.82 (s, 3H), 3.74 (s, 3H), 2.60 (m, 2H), 1.14 (t, J = 7.5 |
| | | Hz, 3H) |
| Syn. Ex. | 529.30 (M + 1)/ | δ 10.03-9.99 (br s, 1H), 7.67 (s, 1H), 7.62 (d, J = 2.3 |
| 91 | 528.18 for | Hz, 1H), 7.42-7.30 (m, 2H) 7.06 (br s, 1H), 6.81 (br s, |
| | C24H28N6O6S | 1H), 6.70 (br s, 1H), 6.06 (br t, J = 5.4 Hz, 1H), 5.68- |
| | | 5.64 (m, 1H), 5.35 (s, 2H), 4.01 (d, J = 5.8 Hz, 2H), |
| | | 3.82 (s, 3H), 3.73 (s, 3H), 2.58 (q, J = 7.4 Hz, 2H), 2.53 |
| | | (d, J = 4.6 Hz, 3H), 1.14 (t, J = 7.6 Hz, 3H) |
| Syn. Ex. | 620.20 (M + 1)/ | δ 9.85 (s, 1H), 8.86 (br t, J = 5.6 Hz, 1H), 7.75 (s, 1H), |
| 92 | 619.19 for | 7.68 (d, J = 2.3 Hz, 1H), 7.40 (s, 2H), 7.36-7.32 (m, |
| | C31H30FN5O6S | 2H), 7.22-7.18 (m, 3H), 7.12 (br d, J = 8.4 Hz, 1H), |
| | | 6.84 (s, 1H), 6.70 (s, 1H), 5.59-5.43 (m, 1H), 5.36 (s, |
| | | 2H), 5.27-5.19 (m, 3H), 4.19 (d, J = 5.9 Hz, 2H), 3.63 |
| | | (s, 3H), 2.59 (q, J = 7.5 Hz, 2H), 1.13 (t, J = 7.6 Hz, |
| | | 3H) |
| Syn. Ex. | 606.30 (M + 1)/ | δ 9.85 (s, 1H), 7.71 (s, 1H), 7.69 (d, J = 2.4 Hz, 1H), |
| 93 | 605.19 for | 7.41 (br d, J = 9.3 Hz, 2H), 7.39-7.33 (m, 3H), 7.23- |
| | C30H31N5O7S | 7.19 (m, 3H), 7.12 (d, J = 8.6 Hz, 1H), 6.82 (s, 1H), |
| | | 6.70 (s, 1H), 5.36 (s, 2H), 5.22 (s, 2H), 4.02 (d, J = 5.9 |
| | | Hz, 2H), 3.64 (s, 3H), 3.51 (s, 3H), 2.59 (q, J = 7.5 Hz, |
| | | 2H), 1.14 (t, J = 7.6 Hz, 3H). |
| Syn. Ex. | 651.33 (M + 1)/ | δ 10.14 (br s, 1H), 8.87 (br t, J = 5.5 Hz, 1H), 7.99 (br |
| 94 | 650.20 for | d, J = 3.0 Hz, 1H), 7.75-7.68 (m, 2H), 7.39 (s, 2H), |
| | C31H31FN6O7S | 7.09-7.02 (m, 1H), 6.91 (br d, J = 4.1 Hz, 1H), 6.82- |
| | | 6.62 (m, 3H), 5.59-5.43 (m, 1H), 5.34 (s, 2H), 5.26- |
| | | 5.15 (m, 3H), 4.19 (d, J = 5.9 Hz, 2H), 3.77 (s, 3H), |
| | | 3.65 (br s, 3H), 2.62-2.54 (m, 2H), 1.14 (t, J = 7.6 Hz, |
| | | 3H) |
| Syn. Ex. | 637.26 (M + 1)/ | δ 10.12 (br s, 1H), 8.00 (br d, J = 4.8 Hz, 1H), 7.72- |
| 95 | 636.20 for | 7.68 (m, 2H), 7.42 (br d, J = 5.5 Hz, 1H), 7.37 (s, 1H), |
| | C30H32N6O8S | 7.05 (br s, 1H), 6.92 (br s, 1H), 6.78 (br s, 2H), 6.67 (br |
| | | d, J = 2.4 Hz, 1H), 5.34 (s, 2H), 5.19 (br s, 2H), 4.02 (d, |
| | | J = 5.9 Hz, 2H), 3.77 (s, 3H), 3.66 (br s, 3H), 3.51 (s, |
| | | 3H), 2.59 (q, J = 7.3 Hz, 2H), 1.14 (t, J = 7.6 Hz, 3H) |
| Syn. Ex. | 570.30 (M + 1)/ | δ 9.82 (s, 1H), 8.86 (br t, J = 5.5 Hz, 1H), 7.65 (s, 1H), |
| 96 | 569.17 for | 7.62 (d, J = 2.1 Hz, 1H), 7.46 (dd, J = 2.3, 8.5 Hz, 1H), |
| | C27H28FN5O6S | 7.40 (s, 1H), 7.22-7.05 (m, 1H), 6.54 (s, 1H), 5.59- |
| | | 5.43 (m, 1H), 5.34 (s, 2H), 5.23 (dd, J = 3.4, 15.8 Hz, |
| | | 1H), 4.21-4.15 (m, 4H), 3.76 (br s, 3H), 2.71-2.66 |
| | | (m, 2H), 2.60 (q, J = 7.5 Hz, 2H), 2.00-1.94 (m, 2H), |
| | | 1.14 (t, J = 7.6 Hz, 3H) |
| Syn. Ex. | 556.25 (M + 1)/ | δ 9.82 (s, 1H), 7.62 (s, 2H), 7.45 (br t, J = 8.1 Hz, 2H), |
| 97 | 555.18 for | 7.38 (s, 1H), 7.12 (br d, J = 8.4 Hz, 1H), 6.52 (s, 1H), |
| | C26H29N5O7S | 5.34 (s, 2H), 4.19-4.16 (m, 2H), 4.02 (d, J = 5.9 Hz, |
| | | 2H), 3.77 (s, 3H), 3.51 (s, 3H), 2.71-2.66 (m, 2H), 2.63- |
| | | 2.56 (m, 2H), 2.02-1.93 (m, 2H), 1.14 (t, J = 7.6 Hz, |
| | | 3H) |
| Syn. Ex. | 579.30 (M + 1)/ | δ 7.64 (d, J = 2.3 Hz, 1H), 7.35-7.25 (m, 1H), 7.10 (br |
| 98 | 578.15 for | dd, J = 4.1, 7.4 Hz, 1H), 7.03-6.98 (m, 1H), 6.90 (s, |
| | C25H30N4O8S2 | 1H), 6.79-6.67 (m, 1H), 5.80 (t, J = 5.63 Hz, 1 H), 4.14 |
| | | (d, J = 5.6 Hz, 2H), 3.86 (s, 3H), 3.72 (s, 3H), 2.57 (br |
| | | d, J = 7.5 Hz, 2H), 1.14 (t, J = 7.6 Hz, 3H), 1.11 (s, 9H) |
| Syn. Ex. | 547.26 (M + 1)/ | ¹H NMR (400 MHz, METHANOL-d₄) δ 7.81 (d, J = |
| 99 | 546.12 for | 2.0 Hz, 1H), 7.37-7.28 (d, 1H), 7.01 (d, 2H), 6.86- |
| | C24H23FN4O8S | 6.81 (d, 1H), 6.73 (br s, 1H), s5.70-5.16 (m, 1H), 4.48- |
| | | 4.20 (d, 2H), 3.97-3.92 (d, 3H), 3.80 (s, 3H), 2.62 (q, |
| | | J = 7.6 Hz, 2H), 1.21 (m, 3H) |
| Syn. Ex. | 533.10 (M + 1)/ | δ 10.18 (br s, 1H), 7.70-7.63 (m, 2H), 7.45 (br d, J = |
| 100 | 532.13 for | 1.3 Hz, 1H), 7.31 (br s, 1H), 7.10 (d, J = 1.0 Hz, 1H), |
| | C23H24N4O9S | 6.89 (br s, 1H), 6.84 (s, 1H), 4.18 (d, J = 5.6 Hz, 2H), |
| | | 3.85 (s, 3H), 3.75 (s, 3H), 3.55 (s, 3H), 2.63-2.59 (m, |
| | | 2H), 1.15 (t, J = 7.6 Hz, 3H) |
| Syn. Ex. | 549.20 (M + 1)/ | δ 0.17 (br s, 1H), 7.76 (br s, 1H), 7.64 (d, J = 2.3 Hz, |
| 101 | 548.10 for | 1H), 7.48-7.45 (m, 1H), 7.23 (br d, J = 0.9 Hz, 1H), |
| | C23H24N4O8S2 | 7.16 (s, 1H), 7.12 (br d, J = 8.4 Hz, 1H), 6.85 (br s, 1H), |
| | | 4.25 (d, J = 5.9 Hz, 2H), 3.84 (s, 3H), 3.75 (s, 3H), 3.54 |
| | | (s, 3H), 2.62-2.59 (m, 2H), 1.15 (t, J = 7.6 Hz, 3H) |
| Syn. Ex. | 563.21 (M + 1)/ | δ 10.16 (br s, 1H), 9.18 (br t, J = 5.9 Hz, 1H), 7.64 (d, |
| 102 | 562.10 for | J = 2.3 Hz, 1H), 7.46 (br d, J = 7.1 Hz, 1H), 7.24-7.20 |
| | C24H23FN4O7S2 | (m, 2H), 7.11 (d, J = 8.5 Hz, 1H), 6.85 (s, 1H), 5.63- |
| | | 5.48 (m, 1H), 5.29 (dd, J = 3.6, 15.7 Hz, 1H), 4.41 (d, |
| | | J = 5.9 Hz, 2H), 3.84 (s, 3H), 3.75 (s, 3H), 2.62-2.59 (m, |
| | | 2H), 1.15 (t, J = 7.6 Hz, 3H) |
| Syn. Ex. | 543.20 (M + 1)/ | δ 10.17 (br s, 1H), 9.35 (br t, J = 5.8 Hz, 1H), 7.65 (d, |
| 103 | 542.09 for | J = 2.3 Hz, 1H), 7.45 (br d, J = 2.5 Hz, 1H), 7.28-7.21 |
| | C24H22N4O7S2 | (m, 1H), 7.19 (s, 1H), 7.13-7.09 (m, 1H), 6.85 (br s, |
| | | 1H), 4.35 (d, J = 5.6 Hz, 2H), 4.20 (s, 1H), 3.84 (s, 3H), |
| | | 3.74 (s, 3H), 2.61 (q, J = 7.7 Hz, 2H), 1.15 (t, J = 7.6 |
| | | Hz, 3H) |
| Syn. Ex. | 549.33 (M + 1)/ | δ 10.18 (s, 1 H), 7.64 (br d, J = 2.1 Hz, 2H), 7.50-7.44 |
| 104 | 548.10 for | (s, 1H), 7.28 (br s, 1H), 7.12 (br d, J = 7.6 Hz, 1H), 6.95 |
| | C23H24N4O8S2 | (s, 1H), 6.87 (br s, 1H), 4.10 (br d, J = 6.0 Hz, 2H), 3.84 |
| | | (s, 3H), 3.75 (s, 3H), 3.54 (s, 3H), 2.61 (q, J = 7.4 Hz, |
| | | 2H), 1.15 (t, J = 7.6 Hz, 3H) |
| Syn. Ex. | 563.10 (M + 1)/ | δ 10.19 (s, 1 H), 9.00 (br t, J = 5.75 Hz, 1 H), 7.64 (d, |
| 105 | 562.10 for | J = 2.3 Hz, 1H), 7.48 (dd, J = 2.3, 8.5 Hz, 1H), 7.29 (d, |
| | C24H23FN4O7S2 | J = 1.6 Hz, 1H), 7.13 (d, J = 8.5 Hz, 1H), 6.99 (s, 1H), |
| | | 6.89 (d, J = 1.6 Hz, 1H), 5.63-5.46 (m, 1H), 5.28 (dd, |
| | | J = 3.4, 15.7 Hz, 1H), 4.27 (d, J = 5.9 Hz, 2H), 3.84 (s, |
| | | 3H), 3.75 (s, 3H), 2.61 (d, J = 7.5 Hz, 2H), 1.16 (t, J = |
| | | 7.6 Hz, 3H) |
| Syn. Ex. | 543.20 (M + 1)/ | δ 7.78-7.70 (m, 2H), 7.63 (br t, J = 6.1 Hz, 1H), 7.41- |
| 106 | 542.15 for | 7.19 (m, 2H), 7.09 (td, J = 1.5, 2.6 Hz, 1H), 6.98 (br d, |
| | C25H26N4O8S | J = 7.3 Hz, 2H), 6.76-6.64 (m, 2H), 4.10 (d, J = 6.1 |
| | | Hz, 2H), 3.82 (s, 3H), 3.70 (s, 3H), 3.52 (s, 3H), 2.57 |
| | | (q, J = 7.6 Hz, 2H), 1.15 (t, J = 7.6 Hz, 3H) |
| Syn. Ex. | 543.33 (M + 1)/ | δ 10.54 (s, 1 H), 10.05 (br s, 1 H), 8.50 (d, J = 2.5 Hz, |
| 107 | 542.13 for | 1H), 8.20 (dd, J = 2.6, 8.8 Hz, 1H), 7.64 (d, J = 2.0 Hz, |
| | C25H23FN4O7S | 1H), 7.49-7.41 (s, 1H), 7.13 (br d, J = 8.6 Hz, 2H), |
| | | 6.93-6.85 (s, 1H), 6.63 (br s, 1H), 5.81-5.65 (m, 1H), |
| | | 5.50-5.42 (m, 1H), 3.82 (s, 3H), 3.77 (s, 3H), 2.61 (br |
| | | d, J = 7.5 Hz, 2H), 1.15 (t, J = 7.6 Hz, 3H) |
| Syn. Ex. | 523.21 (M + 1)/ | δ 10.99 (s, 1H), 8.37 (d, J = 2.4 Hz, 1H), 8.17 (s, 1H), |
| 108 | 522.12 for | 8.06 (dd, J = 2.8, 8.9 Hz, 1H), 7.65 (d, J = 2.3 Hz, 1H), |
| | C25H22N4O7S | 7.28-7.16 (m, 1H), 7.05 (d, J = 8.8 Hz, 1H), 6.93 (br |
| | | d, J = 7.1 Hz, 1H), 6.62 (br s, 1H), 6.42 (br s, 1H), 4.46 |
| | | (s, 1H), 3.81 (s, 3H), 3.71 (s, 3H), 2.55 (m, 2H), 1.14 (t, |
| | | J = 7.6 Hz, 3H) |
| Syn. Ex. | 529.26 (M + 1)/ | ¹H NMR (400 MHz, METHANOL-d₄) δ 8.25 (d, J = |
| 109 | 528.13 for | 2.6 Hz, 1H), 8.02 (dd, J = 2.5, 8.6 Hz, 1H), 7.80 (d, J = |
| | C24H24N4O8S | 2.3 Hz, 1H), 7.44 (dd, J = 2.2, 8.4 Hz, 1H), 7.07-7.01 |
| | | (m, 2H), 6.68 (d, J = 1.6 Hz, 1H), 6.58 (d, J = 1.5 Hz, |
| | | 1H), 3.96 (s, 3H), 3.85 (s, 3H), 3.75 (s, 3H), 2.66 (d, |
| | | J = 7.6 Hz, 2H), 1.23 (t, J = 7.6 Hz, 3H) |
| Syn. Ex. | 543.29 (M + 1)/ | δ 12.99 (br s, 1H), 8.06 (d, J = 5.1 Hz, 1H), 7.79 (br t, |
| 110 | 542.15 for | J = 6.1 Hz, 1H), 7.71 (d, J = 2.3 Hz, 1H), 7.38 (dd, J = |
| | C25H26N4O8S | 1.8, 8.3 Hz, 1H), 7.08-7.00 (m, 3H), 6.84 (s, 2H), 4.21 |
| | | (d, J = 6.3 Hz, 2H), 3.86 (s, 3H), 3.71 (s, 3H), 3.56 (s, |
| | | 3H), 2.61 (q, J = 7.5 Hz, 2H), 1.16 (t, J = 7.6 Hz, 3H) |
| Syn. Ex. | 557.30 (M + 1)/ | δ 12.99 (br s, 1H), 9.16 (br t, J = 6.1 Hz, 1H), 8.07 (d, |
| 111 | 556.14 for | J = 5.1 Hz, 1H), 7.71 (d, J = 2.3 Hz, 1H), 7.39 (dd, J = |
| | C26H25FN4O7S | 2.0, 8.4 Hz, 1H), 7.07 (d, J = 8.5 Hz, 1H), 7.02 (d, J = |
| | | 5.3 Hz, 1H), 6.99 (br s, 1H), 6.86 (s, 1H), 6.82 (br s, |
| | | 1H), 5.65-5.49 (m, 1H), 5.31 (dd, J = 3.5, 15.6 Hz, |
| | | 1H), 4.38 (d, J = 6.1 Hz, 2H), 3.86 (s, 3H), 3.71 (s, 3H), |
| | | 2.61 (q, J = 7.6 Hz, 2H), 1.16 (t, J = 7.6 Hz, 3H) |
| Syn. Ex. | 526.29 (M + 1)/ | δ 10.80 (br s, 1H), 7.97-7.93 (m, 2H), 7.71 (s, 1H), |
| 112 | 525.13 for | 7.45-7.41 (m, 1H), 7.37 (s, 1H), 7.25 (br d, J = 8.0 Hz, |
| | C24H23N5O7S | 1H), 6.83 (br s, 1H), 6.73 (br s, 1H), 5.37 (s, 2H), 4.41 |
| | | (s, 1H), 4.01 (d, J = 5.8 Hz, 2H), 3.90 (s, 3H), 3.87 (s, |
| | | 3H), 3.51 (s, 3H) |
| Syn. Ex. | 574.49 (M + 1)/ | δ 10.07 (s, 1H), 8.87 (br t, J = 5.7 Hz, 1H), 7.74 (s, 1H), |
| 113 | 573.17 for | 7.63 (d, J = 2.2 Hz, 1H), 7.38 (s, 1H), 7.21-7.04 (m, |
| | C26H28FN5O7S | 1H), 6.88-6.62 (m, 1H), 6.51 (s, 1H), 6.37-6.36 (m, |
| | | 1H), 5.68-5.41 (m, 2H), 5.35 (s, 2H), 5.23 (dd, J = 3.4, |
| | | 15.7 Hz, 1H), 4.18 (d, J = 5.9 Hz, 2H), 4.04 (br t, J = |
| | | 4.5 Hz, 2H), 3.81 (s, 3H), 3.64 (br d, J = 1.1 Hz, 2H), |
| | | 2.62-2.54 (m, 2H), 1.15 (t, J = 7.5 Hz, 3H). |
| Syn. Ex. | 611.20 (M + 1)/ | δ ppm 14.85 (br s, 1 H), 8.85 (br t, J = 5.44 Hz, 1 H), |
| 114 | 610.18 for | 7.74 (s, 1 H), 7.66 (d, J = 2.13 Hz, 1 H), 7.36-7.45 (m, |
| | C27H27FN8O6S | 2 H), 7.22-7.28 (m, 1 H), 6.81 (br s, 1 H), 6.68 (br s, 1 |
| | | H), 5.43-5.58 (m, 1 H), 5.35 (s, 2 H), 5.19-5.31 (m, 3 |
| | | H), 4.18 (d, J = 5.88 Hz, 2 H), 3.74 (br s, 3 H), 2.57- |
| | | 2.64 (m, 2 H), 1.15 (t, J = 7.57 Hz, 3 H) |
| Syn. Ex. | 588.20 (M + 1)/ | δ ppm 9.35 (br s, 1 H), 7.71 (s, 1 H), 7.61 (d, J = 2.25 Hz, |
| 115 | 587.18 for | 1 H), 7.39-7.47 (m, 2 H), 7.37 (s, 1 H), 7.20 (d, J = 8.50 |
| | C25H29N7O8S | Hz, 1 H), 6.85 (s, 1 H), 6.73 (s, 1 H), 5.60 (br s, 2 H), |
| | | 5.36 (s, 2 H), 4.61 (s, 2 H), 4.01 (d, J = 5.88 Hz, 2 H), |
| | | 3.82 (s, 3 H), 3.51 (s, 3 H), 2.60 (q, J = 7.55 Hz, 2 H), |
| | | 1.14 (t, J = 7.57 Hz, 3 H) |
| Syn. Ex. | 596.30 (M + 1)/ | δ ppm 7.65-7.69 (m, 2 H), 7.33-7.43 (m, 3 H), 7.22 |
| 116 | 595.18 for | (d, J = 8.38 Hz, 1 H), 7.11 (s, 2 H), 6.73 (s, 1 H), 6.64 (s, |
| | C27H29N7O7S | 1 H), 5.32 (s, 2 H), 5.14 (s, 2 H), 4.00 (br d, J = 5.75 Hz, |
| | | 2 H), 3.87 (s, 3 H), 3.54 (s, 3 H), 2.59-2.65 (m, 2 H), |
| | | 1.18 (t, J = 7.57 Hz, 3 H) |
| Syn. Ex. | 597.20 (M + 1)/ | δ ppm 14.10 (br s, 1 H), 10.38-10.52 (m, 1 H), 7.71 (s, |
| 117 | 596.18 for | 1 H), 7.65 (d, J = 2.00 Hz, 1 H), 7.49 (br d, J = 7.88 Hz, 1 |
| | C26H28N8O7S | H), 7.34-7.45 (m, 3 H), 6.84 (br s, 1 H), 6.72 (br s, 1 |
| | | H), 5.36 (s, 2 H), 5.27 (s, 2 H), 4.01 (d, J = 5.88 Hz, 2 |
| | | H), 3.86 (s, 3 H), 3.51 (s, 3 H), 2.59-2.65 (m, 2 H), |
| | | 1.16 (t, J = 7.57 Hz, 3 H) |
| Syn. Ex. | 602.20 (M + 1)/ | δ ppm 9.31 (br s, 1 H), 8.86 (br t, J = 4.94 Hz, 1 H), 7.74 |
| 118 | 601.18 for | (s, 1 H), 7.62 (d, J = 2.00 Hz, 1 H), 7.39 (s, 2 H), 7.17 (br |
| | C26H28FN7O7S | d, J = 5.38 Hz, 1 H), 6.83 (br s, 1 H), 6.70 (br s, 1 H), |
| | | 5.41-5.68 (m, 3 H), 5.36 (s, 2 H), 5.23 (dd, J = 15.76, |
| | | 3.38 Hz, 1 H), 4.57 (br s, 2 H), 4.19 (d, J = 5.88 Hz, 2 |
| | | H), 3.82 (s, 3 H), 2.59 (q, J = 7.63 Hz, 2 H), 1.14 (t, |
| | | J = 7.57 Hz, 3 H) |
| Syn. Ex. | 610.20 (M + 1)/ | δ ppm 8.85 (br t, J = 5.38 Hz, 1 H), 7.72 (s, 1 H), 7.66 (d, |
| 119 | 609.18 for | J = 2.25 Hz, 1 H), 7.35-7.42 (m, 2 H), 7.25 (d, J = 8.38 |
| | C28H28FN7O6S | Hz, 1 H), 7.14 (s, 2 H), 6.77 (s, 1 H), 6.66 (s, 1 H), 5.42- |
| | | 5.59 (m, 1 H), 5.33 (s, 2 H), 5.16-5.26 (m, 3 H), 4.17 |
| | | (d, J = 5.88 Hz, 2 H), 3.86 (s, 3 H), 2.63 (q, J = 7.60 Hz, 2 |
| | | H), 1.18 (t, J = 7.57 Hz, 3 H) |
| Syn. Ex. | 531.32 (M + 1)/ | δ ppm 10.89 (br s, 1 H), 8.49 (br s, 1 H), 7.71 (s, 1 H), |
| 120 | 530.16 for | 7.55 (s, 1 H), 7.42 (br d, J = 5.88 Hz, 1 H), 7.37 (s, 1 H), |
| | C23H26N6O7S | 6.87 (br s, 1 H), 6.72 (br s, 1 H), 5.37 (s, 2 H), 4.01 (d, |
| | | J = 5.75 Hz, 2 H), 3.84 (s, 3 H), 3.75 (s, 3 H), 3.52 (s, 3 |
| | | H), 2.76 (q, J = 7.63 Hz, 2 H), 1.19 (t, J = 7.57 Hz, 3 H) |
| Syn. Ex. | 531.20 (M + 1)/ | δ ppm 10.70 (br s, 1 H), 8.26 (br s, 1 H), 8.01 (d, J = 2.20 |
| 121 | 530.16 for | Hz, 1 H), 7.71 (s, 1 H), 7.43 (br t, J = 5.20 Hz, 1 H), 7.37 |
| | C23H26N6O7S | (s, 1 H), 6.63-6.94 (m, 2 H), 5.37 (s, 2 H), 4.01 (d, |
| | | J = 5.87 Hz, 2 H), 3.78 (d, J = 14.43 Hz, 6 H), 3.51 (s, 3 |
| | | H), 2.62 (q, J = 7.54 Hz, 2 H), 1.15 (t, J = 7.58 Hz, 3 H) |
| Syn. Ex. | 624.20 (M + 1)/ | δ ppm 9.94 (s, 1 H), 7.71 (s, 1 H), 7.68 (d, J = 2.20 Hz, 1 |
| 122 | 623.18 for | H), 7.33-7.45 (m, 5 H), 7.05 (br t, J = 8.80 Hz, 2 H), |
| | C30H30FN5O7S | 6.68-6.82 (m, 2 H), 5.36 (s, 2 H), 5.17 (s, 2 H), 4.01 |
| | | (d, J = 5.99 Hz, 2 H), 3.66 (s, 3 H), 3.51 (s, 3 H), 2.59 (q, |
| | | J = 7.58 Hz, 2 H), 1.14 (t, J = 7.58 Hz, 3 H) |
| Syn. Ex. | 607.20 (M + 1)/ | δ ppm 10.02 (br s, 1 H), 8.63 (br s, 1 H), 8.44 (dd, |
| 123 | 606.19 for | J = 4.69, 1.31 Hz, 1 H), 7.64-7.84 (m, 3 H), 7.33-7.48 |
| | C29H30N6O7S | (m, 3 H), 7.07-7.24 (m, 2 H), 6.57-6.76 (m, 2 H), 5.33 |
| | | (s, 2 H), 5.19 (br s, 2 H), 4.01 (d, J = 5.88 Hz, 2 H), 3.69 |
| | | (br s, 3 H), 3.51 (s, 3 H), 2.55-2.62 (m, 2 H), 1.14 (t, |
| | | J = 7.57 Hz, 3 H) |
| Syn. Ex. | 546.36 (M + 1)/ | δ ppm 10.40 (br s, 1 H), 7.61 (s, 1 H), 7.56 (dd, J = 8.19, |
| 124 | 545.14 for | 3.06 Hz, 1 H), 7.43 (t, J = 1.00 Hz, 1 H), 7.38 (s, 1 H), |
| | C24H24FN5O7S | 7.23 (br s, 1 H), 6.51 (br s, 1 H), 5.33 (s, 2 H), 4.16 (t, |
| | | J = 1.00 Hz, 2 H), 4.02 (d, J = 5.88 Hz, 2 H), 3.77 (s, 3 H), |
| | | 3.52 (s, 3 H), 2.64-2.71 (m, 2 H), 1.93-2.01 (m, 2 H) |
| Syn. Ex. | 530.33 (M + 1)/ | δ 9.93 (br s, 1 H), 7.67-7.73 (m, 2 H), 7.44 (br t, J = 4.80 |
| 125 | 529.16 for | Hz, 1 H), 7.37 (s, 1 H), 7.02 (br s, 1 H), 6.93 (br d, |
| | C24H27N5O7S | J = 7.13 Hz, 1 H), 6.84 (br s, 1 H), 6.74 (br s, 1 H), 5.37 |
| | | (s, 2 H), 4.02 (d, J = 5.75 Hz, 2 H), 3.85 (s, 3 H), 3.78 (s, |
| | | 3 H), 3.52 (s, 3 H), 2.65 (q, J = 1.00 Hz, 2 H), 1.19 (t, |
| | | J = 7.63 Hz, 3 H) |
| Syn. Ex. | 544.10 (M + 1)/ | δ 9.93 (br s, 1 H), 7.71 (s, 1 H), 7.43 (br s, 1 H), 7.37 (s, |
| 126 | 543.14 for | 1 H), 7.14 (br s, 1 H), 7.09 (s, 1 H), 6.84 (br s, 1 H), |
| | C24H25N5O8S | 6.74 (br s, 1 H), 5.37 (s, 2 H), 4.54 (t, J = 8.63 Hz, 2 H), |
| | | 4.01 (d, J = 5.88 Hz, 2 H), 3.87 (s, 3 H), 3.73 (s, 3 H), |
| | | 3.52 (s, 3 H), 3.22 (br t, J = 8.63 Hz, 2 H) |
| Syn. Ex. | 598.20 (M + 1)/ | δ 10.14 (br s, 1 H), 9.14 (s, 1 H), 7.72 (s, 1 H), 7.68 (d, |
| 127 | 597.16 for | J = 2.13 Hz, 1 H), 7.51 (br d, J = 8.13 Hz, 1 H), 7.40-7.45 |
| | C26H27N7O8S | (m, 1 H), 7.37 (s, 1 H), 7.31 (br d, J = 8.13 Hz, 1 H), 6.85 |
| | | (s, 1 H), 6.72 (s, 1 H), 5.53 (s, 2 H), 5.37 (s, 2 H), 4.01 |
| | | (d, J = 5.75 Hz, 2 H), 3.79 (s, 3 H), 3.51 (s, 3 H), 2.59- |
| | | 2.65 (m, 2 H), 1.16 (t, J = 7.57 Hz, 3 H) |
| Syn. Ex. | 554.20 (M + 1)/ | δ 9.92 (br s, 1 H), 7.71 (s, 1 H), 7.67 (d, J = 2.25 Hz, 1 |
| 128 | 553.16 for | H), 7.40-7.51 (m, 2 H), 7.37 (s, 1 H), 7.18 (br d, J = 8.00 |
| | C26H27N5O7S | Hz, 1 H), 6.84 (br s, 1 H), 6.73 (br s, 1 H), 5.37 (s, 2 H), |
| | | 4.87 (br s, 2 H), 4.02 (d, J = 5.88 Hz, 2 H), 3.86 (s, 3 H), |
| | | 3.52 (s, 3 H), 3.40 (t, J = 2.31 Hz, 1 H), 2.61 (q, J = 7.50 |
| | | Hz, 2 H), 1.16 (t, J = 7.57 Hz, 3 H) |
| Syn. Ex. | 537.31 (M + 1)/ | δ 8.91 (d, J = 1.25 Hz, 1 H), 8.29-8.36 (m, 2 H), 8.24 |
| 129 | 536.15 for | (br d, J = 7.38 Hz, 1 H), 7.74 (br t, J = 7.63 Hz, 1 H), 7.68 |
| | C25H24N6O6S | (s, 1 H), 7.40 (br s, 1 H), 7.34 (s, 1 H), 6.80 (br s, 1 H), |
| | | 6.70 (s, 1 H), 5.32 (s, 2 H), 3.99 (d, J = 5.75 Hz, 2 H), |
| | | 3.83 (s, 3 H), 3.51 (s, 3 H), 2.53 (s, 3 H) |
| Syn. Ex. | 537.34 (M + 1)/ | δ 8.99 (br d, J = 2.75 Hz, 1 H), 8.44 (br d, J = 7.88 Hz, 1 |
| 130 | 536.15 for | H), 8.27 (d, J = 1.88 Hz, 1 H), 8.10 (br s, 1 H), 7.60- |
| | C25H24N6O6S | 7.69 (m, 2 H), 7.40 (br s, 1 H), 7.34 (s, 1 H), 6.80 (s, 1 |
| | | H), 6.70 (s, 1 H), 5.32 (s, 2 H), 3.99 (d, J = 5.88 Hz, 2 |
| | | H), 3.84 (s, 3 H), 3.51 (s, 3 H), 2.57 (s, 3 H) |
| Syn. Ex. | 537.34 (M + 1)/ | δ 8.91 (br d, J = 4.25 Hz, 1 H), 8.36-8.49 (m, 2 H), 7.79 |
| 131 | 536.15 for | (br t, J = 7.94 Hz, 1 H), 7.67 (s, 1 H), 7.54 (br d, J = 3.25 |
| | C25H24N6O6S | Hz, 1 H), 7.36-7.44 (m, 1 H), 7.34 (s, 1 H), 6.78 (br s, |
| | | 1 H), 6.69 (s, 1 H), 5.31 (s, 2 H), 3.99 (d, J = 5.75 Hz, 2 |
| | | H), 3.85 (s, 3 H), 3.51 (s, 3 H), 2.72 (s, 3 H) |
| Syn. Ex. | 563.32 (M + 1)/ | δ 9.01 (br d, J = 2.88 Hz, 1 H), 8.44 (br d, J = 8.00 Hz, 1 |
| 132 | 562.16 for | H), 8.27 (d, J = 1.50 Hz, 1 H), 8.10 (br s, 1 H), 7.67 (br |
| | C27H26N6O6S | dd, J = 8.19, 4.19 Hz, 1 H), 7.58 (s, 1 H), 7.33-7.43 (m, |
| | | 2 H), 6.45 (s, 1 H), 5.28 (s, 2 H), 4.17-4.21 (m, 2 H), |
| | | 3.99 (br d, J = 5.75 Hz, 2 H), 3.50 (s, 3 H), 2.63 (br t, |
| | | J = 6.32 Hz, 2 H), 2.57 (s, 3 H), 1.91-1.99 (m, 2 H) |
| Syn. Ex. | 528.20 (M + 1)/ | δ 9.97 (br s, 1 H), 7.79 (br d, J = 6.36 Hz, 1 H), 7.62 (s, |
| 133 | 527.15 for | 2 H), 7.35-7.48 (m, 2 H), 6.99-7.25 (m, 2 H), 6.50 (br |
| | C24H25N5O7S | s, 1 H), 5.33 (s, 2 H), 4.17 (br t, J = 4.83 Hz, 2 H), 4.02 |
| | | (br d, J = 5.75 Hz, 2 H), 3.80 (s, 3 H), 3.51 (s, 3 H), 2.63- |
| | | 2.72 (m, 2 H), 1.93-2.02 (m, 2 H) |
| Syn. Ex. | 514.33 (M + 1)/ | δ 10.80 (br s, 1 H), 7.73 (dd, J = 7.82, 1.69 Hz, 1 H), 7.66 |
| 134 | 513.13 for | (s, 1 H), 7.59-7.64 (m, 1 H), 7.38-7.47 (m, 1 H), 7.36 |
| | C23H23N5O7S | (s, 1 H), 7.21 (br d, J = 6.00 Hz, 1 H), 7.00-7.09 (m, 1 |
| | | H), 6.72 (br s, 1 H), 5.34 (s, 2 H), 4.70 (br t, J = 8.88 Hz, |
| | | 2 H), 4.01 (d, J = 5.75 Hz, 2 H), 3.78 (s, 3 H), 3.52 (s, 3 |
| | | H), 3.11 (br t, J = 9.01 Hz, 2 H) |
| Syn. Ex. | 552.37 (M + 1)/ | δ 8.11 (s, 1 H), 7.76 (br d, J = 8.63 Hz, 1 H), 7.65 (s, 1 |
| 135 | 551.13 for | H), 7.47 (s, 1 H), 7.22 (d, J = 8.76 Hz, 1 H), 6.59-6.94 |
| | C23H23F2N5O7S | (m, 3 H), 5.38 (s, 2 H), 4.14 (s, 2 H), 3.90 (s, 3 H), 3.84 |
| | | (s, 3 H), 3.62 (s, 3 H), 2.02 (s, 1 H) |
| Syn. Ex. | 530.20 (M + 1)/ | δ 9.52 (s, 1 H), 7.65-7.75 (m, 2 H), 7.43 (br s, 1 H), |
| 136 | 529.16 for | 7.37 (s, 1 H), 7.00 (s, 1 H), 6.89 (br d, J = 7.63 Hz, 1 H), |
| | C24H27N5O7S | 6.84 (s, 1 H), 6.75 (s, 1 H), 5.37 (s, 2 H), 4.07 (q, J = 7.00 |
| | | Hz, 2 H), 4.01 (d, J = 5.88 Hz, 2 H), 3.85 (s, 3 H), 3.51 |
| | | (s, 3 H), 2.25-2.38 (m, 3 H), 1.21 (t, J = 6.94 Hz, 3 H) |
| Syn. Ex. | 530.20 (M + 1)/ | δ 11.53 (s, 1 H) 7.68 (d, J = 2.13 Hz, 1 H) 7.64 (s, 1 H) |
| 137 | 529.16 for | 7.58 (s, 1 H) 7.42-7.47 (m, 2 H) 7.37 (s, 1 H) 7.08 (d, |
| | C24H27N5O7S | J = 8.63 Hz, 1 H) 6.86 (s, 1 H) 5.32 (s, 2 H) 4.02 (d, |
| | | J = 5.75 Hz, 2 H) 3.86 (s, 3 H) 3.75 (s, 3 H) 3.51 (s, 3 H) |
| | | 2.60 (q, J = 7.50 Hz, 2 H) 1.14 (t, J = 7.57 Hz, 3 H). |
| Syn. Ex. | 572.20 (M + 1)/ | δ 8.52 (br s, 1 H), 7.71 (s, 1 H), 7.39-7.52 (m, 2 H), |
| 138 | 571.17 for | 7.37 (s, 1 H), 6.74-6.88 (m, 4 H), 5.36 (s, 2 H), 4.34 |
| | C26H29N5O8S | (br s, 2 H), 4.11 (br s, 2 H), 4.01 (d, J = 5.88 Hz, 2 H), |
| | | 3.86 (s, 3 H), 3.51 (s, 3 H), 1.82-1.97 (m, 2 H), 1.74- |
| | | 1.79 (m, 2 H), 1.63-1.70 (m, 2 H) |
| Syn. Ex. | 556.20 (M + 1)/ | δ 7.67 (s, 1 H), 7.47 (s, 1 H), 7.40-7.45 (m, 1 H), 7.35 |
| 139 | 555.18 for | (s, 1 H), 6.60 (s, 1 H), 6.58 (s, 1 H), 6.50 (s, 1 H), 6.04 |
| | C26H29N5O7S | (br s, 1 H), 5.29 (s, 2 H), 4.01 (d, J = 5.88 Hz, 2 H), 3.83 |
| | | (s, 3 H), 3.63 (s, 3 H), 3.51 (s, 3 H), 2.67 (br dd, J = 3.56, |
| | | 1.56 Hz, 2 H), 2.61 (br s, 2 H), 1.70 (br t, J = 3.06 Hz, 4 |
| | | H) |
| Syn. Ex. | 556.20 (M + 1)/ | δ 9.75 (s, 1 H), 7.69 (d, J = 8.00 Hz, 1 H), 7.62 (s, 1 H), |
| 140 | 555.18 for | 7.44 (br s, 1 H), 7.38 (s, 1 H), 7.04 (s, 1 H), 6.93 (br d, |
| | C26H29N5O7S | J = 8.00 Hz, 1 H), 6.50 (s, 1 H), 5.34 (s, 2 H), 4.20 (br t, |
| | | J = 4.88 Hz, 2 H), 4.02 (d, J = 5.75 Hz, 2 H), 3.81 (s, 3 H), |
| | | 3.51 (s, 3 H), 2.60-2.71 (m, 4 H), 2.31-2.34 (m, 2 H), |
| | | 1.94-2.03 (m, 3 H) |
| Syn. Ex. | 548.37 (M + 1)/ | δ 10.49 (br s, 1 H), 7.68 (br s, 1 H), 7.62 (s, 1 H), 7.32- |
| 141 | 547.15 for | 7.48 (m, 3 H), 7.09-7.19 (m, 1 H), 6.91-6.96 (m, 1 |
| | C24H26FN5O7S | H), 5.40 (br s, 2 H), 4.05-4.15 (m, 2 H), 4.01 (br d, |
| | | J = 5.88 Hz, 3 H), 3.72 (br s, 3 H), 3.52 (s, 3 H), 2.53- |
| | | 2.59 (m, 2 H), 1.14 (t, J = 7.57) |
| Syn. Ex. | 544.43 (M + 1)/ | δ 11.51 (s, 1H) 7.68 (d, J = 2.00 Hz, 1H) 7.64 (s, 1H) |
| 142 | 543.18 for | 7.39-7.49 (m, 3H) 7.35 (s, 1H) 7.05 (d, J = 8.25 Hz, 1 |
| | C₂₅H₂₉N₅O₇S | H) 6.90 (s, 1H) 5.31 (s, 2H) 4.07 (q, J = 6.92 Hz, 2H) |
| | | 4.02 (d, J = 5.75 Hz, 2H) 3.72 (s, 3H) 3.52 (s, 3H) 2.59 |
| | | (q, J = 7.67 Hz, 2H) 1.40 (t, J = 6.88 Hz, 3H) 1.14 (t, |
| | | J = 7.63 Hz, 3H) |
| Syn. Ex. | 575.26 (M + 1)/ | δ = 9.18 (t, J = 5.8 Hz, 1H), 8.15 (s, 1H), 7.64 (s, 1H), |
| 143 | 574.10 for | 7.20 (s, 1H), 7.11-6.66 (m, 3H), 5.69-5.46 (m, 1H), |
| | C25H23FN4O7S2 | 5.29 (dd, J = 3.5, 15.8 Hz, 1H), 4.41 (d, J = 6.0 Hz, 2H), |
| | | 3.87 (s, 3H), 3.73 (s, 3H), 3.01-2.70 (m, 4H), 2.07- |
| | | 1.90 (m, 2H) |
| Syn. Ex. | 555.28 (M + 1)/ | δ = 10.54 (s, 1H), 9.84-9.74 (m, 1H), 8.50 (d, J = 2.5 |
| 144 | 554.13 for | Hz, 1H), 8.20 (dd, J = 2.6, 8.9 Hz, 1H), 7.65 (s, 1H), |
| | C₂₆H₂₃FN₄O₇S | 7.13 (d, J = 8.8 Hz, 1H), 7.09-6.54 (m, 3H), 5.84-5.55 |
| | | (m, 1H), 5.47 (dd, J = 3.8, 15.5 Hz, 1H), 3.86 (s, 3H), |
| | | 3.78 (s, 3H), 2.96-2.75 (m, 4H), 2.14-1.91 (m, 2H) |
| Syn. Ex. | 502.36 (M + 1)/ | δ = 11.59 (s, 1H), 7.86 (dd, J = 1.4, 7.9 Hz, 1H), 7.65 |
| 145 | 501.13 for | (s, 1H), 7.62-7.49 (m, 2H), 7.49-7.40 (m, 1H), 7.37 |
| | C22H23N5O7S | (s, 1H), 7.16 (d, J = 8.3 Hz, 1H), 7.08 (t, J = 7.6 Hz, |
| | | 1H), 6.84 (s, 1H), 5.33 (s, 2H), 4.02 (d, J = 5.8 Hz, 2H), |
| | | 3.87 (s, 3H), 3.79 (s, 3H), 3.52 (s, 3H) |
| Syn. Ex. | 516.37 (M + 1)/ | δ = 11.59 (s, 1H), 8.87 (t, J = 5.8 Hz, 1H), 7.85 (dd, J = |
| 146 | 515.13 for | 1.5, 7.8 Hz, 1H), 7.67 (s, 1H), 7.59-7.46 (m, 2H), 7.38 |
| | C23H22FN5O6S | (s, 1H), 7.25-6.96 (m, 2H), 6.83 (s, 1H), 5.69-5.41 |
| | | (m, 1H), 5.32 (s, 2H), 5.23 (dd, J = 3.4, 15.6 Hz, 1H), |
| | | 4.19 (d, J = 5.8 Hz, 2H), 3.85 (s, 3H), 3.77 (s, 3H) |
| Syn. Ex. | 542.44 (M + 1)/ | δ = 8.24 (s, 1H), 7.62 (d, J = 9.0 Hz, 1H), 7.44 (s, 1H), |
| 147 | 541.16 for | 7.36 (s, 1H), 7.17 (s, 1H), 6.86 (s, 1H), 6.67 (s, 1H), |
| | C25H27N5O7S | 5.28 (s, 2H), 4.02 (d, J = 5.8 Hz, 1H), 3.84 (s, 2H), 3.65 |
| | | (s, 2H), 3.52 (s, 2H), 2.92-2.72 (m, 3H), 2.13-1.86 |
| | | (m, 2H). |
| Syn. Ex. | 561.29 (M + 1)/ | δ = 7.69-7.83 (m, 1 H) 7.62-7.67 (m, 1 H) 6.86-7.21 |
| 148 | 560.10 for | (m, 3 H) 6.63-6.83 (m, 1 H) 4.25 (d, J = 6.00 Hz, 2 H) |
| | C24H24N4O8S2 | 3.88 (s, 3 H) 3.74 (s, 3 H) 3.55 (s, 3 H) 2.76-2.94 (m, |
| | | 4 H) 1.91-2.05 (m, 2 H) |
| Syn. Ex. | 549.30 (M + 1)/ | δ = 11.65 (s, 1 H) 7.64-7.79 (m, 4 H) 7.41 (s, 1 H) 6.97- |
| 149 | 548.60 for | 7.15 (m, 2 H) 4.20 (d, J = 1.00 Hz, 2 H) 3.79 (s, 3 H) |
| | C23H24N4O8S2 | 3.75 (s, 3 H) 3.53 (s, 3 H) 2.55-2.68 (m, 2 H) 1.17 (t, |
| | | J = 1.00 Hz, 3 H) |
| Syn. Ex. | 516.4 (M + 1)/ | δ = 7.62 (s, 2 H) 7.44 (t, J = 1.00 Hz, 1 H) 7.35 (s, 1 H) |
| 150 | 515.5 for | 7.12-7.30 (m, 2 H) 6.90 (d, J = 1.00 Hz, 1 H) 6.71 (s, 1 |
| | C23H25N5O7S | H) 5.30 (s, 2 H) 4.03 (d, J = 1.00 Hz, 2 H) 3.83-3.87 (m, |
| | | 3 H) 3.66 (s, 3 H) 3.52 (s, 3 H) 2.25 (s, 3 H) |
| Syn. Ex. | 530.3 (M + 1)/ | δ = 7.76 (dd, J = 7.75, 1.38 Hz, 1 H) 7.63 (s, 1 H) 7.44 |
| 151 | 529.12 for | (br s, 1 H) 7.36 (s, 1 H) 7.13 (br s, 1 H) 7.01 (br s, 1 H) |
| | C23H23N5O8S | 6.40 (br s, 1 H) 5.28 (s, 2 H) 4.32 (s, 4 H) 4.02 (d, J = 5.75 |
| | | Hz, 2 H) 3.77 (s, 3 H) 3.52 (s, 3 H) |

### Example 152

### KAT6A AlphaLISA Inhibition Screening Assay

The Kat6a inhibitory activity of test compounds was determined using an Alpha Screen-based detection method. The assay reactions were conducted in a volume of 10 μL in Alpha Plate, White 384 well plate (cat #6008280, Perkin Elmer). The enzymatic reactions were performed in assay buffer pH 8.0 (50 mM Tris-HCl, 0.1 mM EDTA, 0.01% Tween-20, 1 mM Dithiothreitol, 0.1% BSA (fatty acid free) and 330 nM TSA (Trichostatin A)). 10 μL reaction volume consisting of 25 nM of Recombinant KAT6A/MOZ (488-778) protein (Active motif, Catalog #81223), 400 nM Acetyl coenzyme A (Catalog #A2056, Sigma), 200 nM of Histone H3 peptide [(amino acids 1-21), biotin-labeled (BPS Biosciences, Catalog #52011)]. Test compounds were screened at different doses starting with 10 μM, 3 fold dilution and 10 point Dose response curve. Enzyme and compounds solution were pre-incubated in assay plate for 60 or 10 min at room temperature then substrate and Acetyl coenzyme A solution were added to the plate. After addition, the plate was sealed with adhesive seals and incubated for 120 minutes at room temperature.

After 120 minutes of incubation, 5 L (10 g/mL) of AlphaLISA anti-acetyl-Lysine acceptor beads (Perkin Elmer, Catalog #AL143C) were added to the plate and it was incubated for 60 minutes at room temperature. Then 10 L (10 g/mL) of Alpha Streptavidin donor beads (Perkin Elmer, Catalog #6760002S) were added to the plate which was further incubated for 60 minutes at room temperature. After incubation, the alpha signal was recorded by using Perkin Elmer Envision multi-mode reader. Percent inhibition of enzyme activity by test compounds were determined relative to vehicle control. Results are shown in Table 2 below.

TABLE 2
Kat6A % Inhibition.

| | Average Kat6A % | Average Kat6A % |
|---|---|---|
| Syn. Ex. # | inhibition @ 10 μM | inhibition @ 1 μM |
| 1 | 100 | 100 |
| 2 | 100 | 71 |
| 3 | 100 | 100 |
| 4 | 100 | 100 |
| 5 | 100 | 83 |
| 6 | 100 | 99 |
| 7 | 99 | 97 |
| 8 | 100 | 99 |
| 9 | 100 | 61 |
| 10 | 100 | 100 |
| 11 | 100 | 100 |
| 12 | 97 | 38 |
| 13 | 100 | 99 |
| 14 | 100 | 100 |
| 15 | 100 | 100 |
| 16 | 98 | 98 |
| 17 | 100 | 99 |
| 18 | 98 | 97 |
| 19 | 98 | 100 |
| 20 | 100 | 100 |
| 21 | 99 | 100 |
| 22 | 100 | 100 |
| 23 | 100 | 100 |
| 24 | 100 | 99 |
| 25 | 100 | 100 |
| 26 | 100 | 99 |
| 27 | 100 | 100 |
| 28 | 100 | 100 |
| 29 | 100 | 100 |
| 30 | 100 | 100 |
| 31 | 99 | 86 |
| 32 | 97 | 75 |
| 33 | 100 | 96 |
| 34 | 100 | 86 |
| 35 | 100 | 100 |
| 36 | 97 | 93 |
| 37 | 100 | 100 |
| 38 | 99 | 100 |
| 39 | 100 | 92 |
| 40 | 99 | 99 |
| 41 | 100 | 87 |
| 42 | 100 | 93 |
| 43 | 98 | 96 |
| 44 | 100 | 97 |
| 45 | 99 | 85 |
| 46 | 100 | 98 |
| 47 | 96 | 98 |
| 47a | 100 | 100 |
| 47b | 100 | 96 |
| 48 | 100 | 96 |
| 49 | 100 | 94 |
| 50 | 96 | 98 |
| 51 | 100 | 89 |
| 52 | 100 | 100 |
| 53 | 100 | 98 |
| 54 | 97 | 97 |
| 55 | 100 | 100 |
| 56 | 99 | 78 |
| 57 | 100 | 70 |
| 58 | 97 | 91 |
| 59 | 100 | 100 |
| 60 | 100 | 93 |
| 61 | 100 | 60 |
| 62 | 100 | 100 |
| 63 | 96 | 95 |
| 64 | 100 | 94 |
| 65 | 100 | 100 |
| 66 | 100 | 93 |
| 67 | 100 | 100 |
| 68 | 100 | 96 |
| 69 | 99 | 96 |
| 70 | 100 | 100 |
| 71 | 100 | 100 |
| 72 | 100 | 100 |
| 73 | 100 | 100 |
| 74 | 100 | 100 |
| 75 | 92 | 59 |
| 76 | 96 | 74 |
| 77 | 98 | 96 |
| 78 | 100 | 100 |
| 79 | 100 | 99 |
| 80 | 100 | 100 |
| 81 | 99 | 91 |
| 82 | 99 | 94 |
| 83 | 99 | 98 |
| 84 | 100 | 100 |
| 85 | 95 | 91 |
| 86 | 100 | 86 |
| 87 | 100 | 95 |
| 88 | 98 | 99 |
| 89 | 100 | 100 |
| 90 | 100 | 99 |
| 91 | 100 | 99 |
| 92 | 100 | 100 |
| 93 | 98 | 90 |
| 94 | 100 | 100 |
| 95 | 100 | 100 |
| 96 | 100 | 100 |
| 97 | 100 | 100 |
| 98 | 92 | 78 |
| 99 | 100 | 91 |
| 100 | 100 | 92 |
| 101 | 100 | 100 |
| 102 | 100 | 99 |
| 103 | 99 | 97 |
| 104 | 92 | 25 |
| 105 | 99 | 68 |
| 106 | 44 | 10 |
| 107 | 100 | 100 |
| 108 | 100 | 99 |
| 109 | 98 | 95 |
| 110 | 58 | 9 |
| 111 | 54 | 0 |
| 112 | 98 | 70 |
| 113 | 100 | 98 |
| 114 | 100 | 100 |
| 115 | 100 | 100 |
| 116 | 100 | 100 |
| 117 | 100 | 100 |
| 118 | 100 | 97 |
| 119 | 100 | 100 |
| 120 | 100 | 100 |
| 121 | 94 | 100 |
| 122 | 100 | 100 |
| 123 | 100 | 100 |
| 124 | 100 | 100 |
| 125 | 101 | 100 |
| 126 | 100 | 101 |
| 127 | 100 | 101 |
| 128 | 100 | 101 |
| 129 | 102 | 102 |
| 130 | 102 | 100 |
| 131 | 100 | 99 |
| 132 | 99 | 100 |
| 133 | 103 | 100 |
| 134 | 101 | 97 |
| 135 | 97 | 96 |
| 136 | 99 | 99 |
| 137 | 89 | 98 |
| 138 | 114 | 111 |
| 139 | 114 | 119 |
| 140 | 104 | 106 |
| 141 | 91 | 104 |
| 142 | 99 | 110 |
| 143 | 99 | 101 |
| 144 | 99 | 101 |
| 145 | 108 | 108 |
| 146 | 101 | 99 |
| 147 | 101 | 101 |
| 148 | 117 | 115 |
| 149 | 102 | 101 |
| 150 | 103 | 101 |

### Example 153

### KAT6A and Kat6B IC50 Using FLAG-Tagged Kat6A and GST-Tagged Kat6B

Compounds were suspended in as a stock of 10 mM in DMSO. Compounds were diluted and tested in 10-doses with 3-fold serial dilutions starting with 10 μM as the highest concentration. Compounds were preincubated in the reaction mixture with the enzyme (5 nM KAT6A, amino acids 488-778 with an N-terminal FLAG tag incorporated; 10 nM KAT61B protein, amino acids 657-1069 with an N-terminal GST tag). This preincubation was for 60 minutes prior to the addition of substrate. Inhibition of KAT6A/MSYT3 activity was tested on a Histone H3 peptide substrate and inhibition of KAT6B/MYST4 activity was tested on a Histone H4 peptide substrate, both in the presence of 1 μM Acetyl-CoA. Reactions were performed in reaction buffer 50 mM Tris-HCl, pH 8.0, 50 mM NaCl, 0.1 mM EDTA, 1 mM EDTA, 1% DMSO and the reactions initiated with the addition of Acetyl-CoA as a co-factor, with histone acetylation measured with a radiometric activity assay using a radioisotope-based filtration assay. Inhibition of acetylation was calculated using with standard curve-fitting model and IC₅₀ determination. Results are shown in Table 3.

TABLE 3
IC₅₀ of selected compounds assayed for inhibition of Kat6A and Kat6B.
This data demonstrates that a substituent on the 5-position of thiazoles (Syn. Ex.
101, Syn. Ex. 102), 5-position of pyridine (Syn. Ex. 107) and 4-position of pyrazole (Syn.
Ex. 52, Syn. Ex. 50, Syn. Ex. 7, Syn. Ex. 14, Syn. Ex. 15, Syn. Ex. 25) result in poor Kat6B
potency and thus confer selectivity against Kat6B. For comparison, data for known
unsubstituted heteroaryl compounds (WO 2023/088233 and WO 2020/254946) is also provided.

| Synthetic example/ | Biochemical assay:Kat6A | Biochemical assay:Kat6B |
|---|---|---|
| reference | IC₅₀ (nM) | IC₅₀ (nM) |
| Compound 59, WO | 1 | 25.5 |
| 2023/088233 | | |
| Syn. Ex. 101 | 4 | 2490 |
| Syn. Ex. 102 | 2 | 2530 |
| Compound 9, WO | 1 | 332 |
| 2023/088233 | | |
| Syn. Ex. 107 | 2 | 1630 |
| 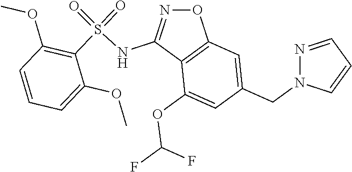 [SMILES: COc1cccc(OC)c1S(=O)(=O)Nc1noc2cc(Cn3cccn3)cc(OC(F)F)c12] | 2 | 144 |
| Claim 83 of WO | | |
| 2023/088233 | | |
| Syn. Ex. 52 | 12 | 3670 |
| Syn. Ex. 50 | 34 | 2396 |
| Example 98, WO | 1 | 40 |
| 2020/254946 | | |
| Example 45, WO | 1 | 24 |
| 2020/254946 | | |
| Syn. Ex. 7 | 22 | 571 |
| Syn. Ex. 14 | 2 | 878 |
| Syn. Ex. 15 | 5 | 1025 |
| Syn. Ex. 25 | 2 | 722 |

### Example 154

### Cell Titer-Glo® Luminescent Cell Viability Assay in ZR-75-1 Cells

The CellTiter-Glo® Luminescent Cell Viability Assay is a homogeneous method employed to determine the number of metabolically active cells in culture. On day 0, ZR-75-1 cells were detached completely from the culture flasks using 0.25% trypsin-EDTA (Catalog #25200056, Gibco). ZR-75-1 cells were seeded into white opaque-96 well culture plate (Catalog #6005680, Perkin Elmer) at a density of 1500 cells per well in 200 μL of RPMI 1640 medium (Catalog #A1049101, Gibco) supplemented with 10% Fetal Bovine Serum (Catalog #16000044, Gibco) and 1× Penicillin-Streptomycin (Catalog #15140122, Gibco). The plates were incubated at 37° C. with 5% CO₂ for 24 hours. On day 1, the spent media was removed from each well of the plate and 180 μL of fresh RPMI 1640 complete media was added. Test compounds were diluted in 100% DMSO followed by complete media. 20 μL of media containing compounds were added to each well so that the final DMSO concentration was 0.5%. The test compounds were tested for 10 points DRC (10 μM top concentration, 3-fold dilution). The assay plates were incubated at 37° C. with 5% CO₂ for 48 hours. The test compounds and media were replenished every 48 hours till day 10.

On day 10, the CellTiter-Glo® 2.0 reagent (Catalog #G9242, Promega) was thawed and equilibrated to room temperature. Assay plates were removed from the incubator and equilibrated to room temperature for about 30 minutes. 100 μL of spent media was removed from each well and then 50 μL of CellTiter-Glo® 2.0 Reagent was added. The plates were incubated on an orbital shaker at room temperature for 10 minutes to induce cell lysis and stabilize luminescent signal. The luminescent signal was recorded using Perkin Elmer Envision multi-mode plate reader. Percent cytotoxicity induced by test compounds was determined relative to vehicle control. EC₅₀ value for test compounds was calculated using GraphPad Prism software (four parameter-variable slope equation). Results are shown in Table 4 below.

TABLE 4
ZR-75-1 cell viability ranges for
selected compounds provided herein.

| Syn. Ex. # | ZR-75-1: EC₅₀ (μM) |
|---|---|
| 7 | A |
| 14 | A |
| 15 | B |
| 19 | A |
| 23 | A |
| 25 | A |
| 46 | B |
| 47 | B |
| 50 | B |
| 51 | B |
| 52 | B |
| 53 | C |
| 54 | A |
| 55 | A |
| 56 | B |

A < 0.1 μM; B 0.1-1 μM; C 1-10 μM.

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A compound of Formula I: 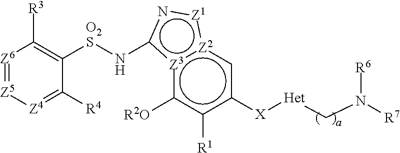
or a pharmaceutically acceptable derivative thereof, wherein:
a is 0-2;
R¹ and R² are selected from (i) or (ii):
(i) R¹ is H, alkyl, haloalkyl or halo; and R² is alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is CR¹⁶;
Z⁵ is CR⁵;
Z⁶ is N or CH;
R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

2. A compound of Formula I: 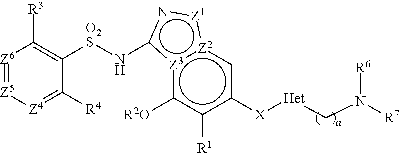
or a pharmaceutically acceptable derivative thereof, wherein:
a is 0-2;
R¹ is H, alkyl, haloalkyl or halo;
R² is alkyl, cycloalkyl or haloalkyl;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is CR²²R²³, where R²² and R²³ are each independently H or alkyl;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷; and
R¹⁷ is alkoxy, alkenyloxy, alkynyloxy or —O—(CH₂)₂—R¹⁹; where R¹⁹ is —OR⁹ or —NR⁹R¹⁰.

3. A compound of Formula II: 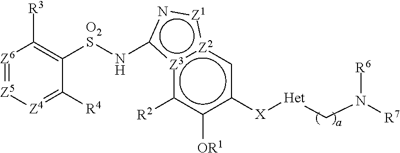
or a pharmaceutically acceptable derivative thereof, wherein:
a is 0-2;
R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR²OR²¹.

4. A compound of Formula II: 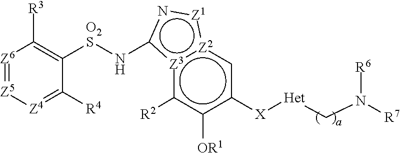
or a pharmaceutically acceptable derivative thereof, wherein:
a is 0-2;
R¹ and R² are selected from (i) or (ii);
(i) R¹ is alkyl, cycloalkyl or haloalkyl; and R² is H, alkyl, haloalkyl, alkoxy, cycloalkoxy, haloalkoxy or halo, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is CR¹⁶;
Z⁵ is CR⁵;
Z⁶ is N or CH;
R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

5. A compound of Formula II: 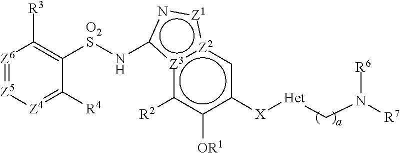
or a pharmaceutically acceptable derivative thereof, wherein:
a is 0-2;
R¹ and R² are selected from (i) or (ii):
(i) R¹ is alkyl, cycloalkyl or haloalkyl, and R² is H, alkyl, haloalkyl, alkoxy, cycloalkoxy, haloalkoxy or halo, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O or S(O)ₓ, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

6. A compound of Formula II: 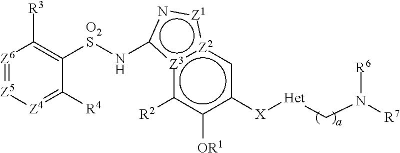
or a pharmaceutically acceptable derivative thereof, wherein:
a is 0-2;
R¹ is alkyl, cycloalkyl or haloalkyl;
R² is H, alkyl, haloalkyl, alkoxy, cycloalkoxy, haloalkoxy or halo;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is CR²²R²³, where R²² and R²³ are each independently H or alkyl;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷; and
R¹⁷ is alkoxy, alkenyloxy, alkynyloxy or —O—(CH₂)₂—R¹⁹; where R¹⁹ is —OR⁹ or —NR⁹R¹⁰.

7. A compound of Formula III: 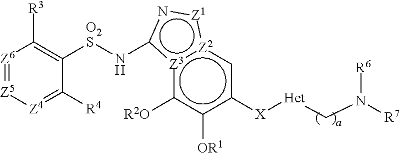
or a pharmaceutically acceptable derivative thereof, wherein:
a is 0-2;
R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR²OR²¹.

8. A compound of Formula III: 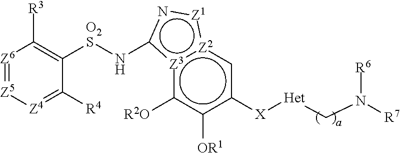
or a pharmaceutically acceptable derivative thereof, wherein:
a is 0-2;
R¹ and R² are selected from (i) or (ii):
(i) R¹ and R² are each independently alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R¹¹, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴ is CR¹⁶;
Z⁵ is CR⁵;
Z⁶ is N or CH;
R⁵ and R¹⁶ together form alkylene or heteroalkylene;
X is O, S(O)ₓ or CH₂, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

9. A compound of Formula III: 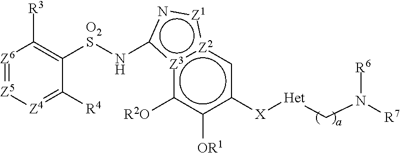
or a pharmaceutically acceptable derivative thereof, wherein:
a is 0-2;
R¹ and R² are selected from (i) or (ii):
(i) R¹ and R² are each independently alkyl, cycloalkyl or haloalkyl, or
(ii) R¹ and R² together form alkylene;
R³ is —O—(CH₂)_{b}—R⁸;
b is 1-4;
R⁸ is H, CN, OH, alkoxy, cycloalkyl, heterocyclyl, aryl, heteroaryl, NR⁹R¹⁰, C(O)R″, C(═NH)(R¹²) or C(═N—O—R⁹)(R¹²);
R⁹ and R¹⁰ are each independently H, alkyl or C(O)R¹³; where R¹³ is alkyl, alkoxy or NR¹⁴R¹⁵;
R¹¹ is hydroxy, alkyl, alkoxy or NR¹⁴R¹⁵;
R¹² is alkyl or NR¹⁴R¹⁵;
R¹⁴ and R¹⁵ are each independently H, OH, alkoxy or alkyl, or together with the nitrogen atom to which they are attached form heterocyclyl;
R⁴ is H or alkoxy;
Z¹, Z² and Z³ are selected from (i), (ii) or (iii):
(i) Z¹ is N, one of Z² and Z³ is C and the other of Z² and Z³ is N,
(ii) Z¹ is NH or O, and Z² and Z³ are C, or
(iii) Z¹ is CH, one of Z² and Z³ is C and the other of Z² and Z³ is N;
Z⁴, Z⁵ and Z⁶ are selected from (i), (ii), (iii) or (iv):
(i) Z⁴ is N, Z⁵ is CR⁵, and Z⁶ is CH,
(ii) Z⁴ is CR¹⁶, Z⁵ is N, and Z⁶ is CH,
(iii) Z⁴ is CR¹⁶, Z⁵ is CR⁵, and Z⁶ is N, or
(iv) Z⁴ is CR¹⁶, Z⁵ is CR⁵, and Z⁶ is CH;
R⁵ and R¹⁶ are selected from (i) or (ii):
(i) R⁵ and R¹⁶ are each independently H, cyano, halo, alkoxy, amino, alkylamino, dialkylamino, alkyl, alkenyl, alkynyl or haloalkyl, or
(ii) R⁵ and R¹⁶ together form alkylene or heteroalkylene;
X is O or S(O)ₓ, where x is 0-2;
Het and R⁶ are selected from (i) or (ii):
(i) Het is a 5- or 6-membered heteroaryl, and R⁶ is H, alkyl or haloalkyl, or
(ii) Het is a 5- or 6-membered heteroaryl, R⁶ is —(CH₂)₁₋₂—, and R⁶ and Het together with the atoms to which they are attached form a 5,5-, 5,6-, 5,7-, 6,5-, 6,6- or 6,7-bicyclic ring system;
R⁷ is —C(O)R¹⁷, —S(O)R¹⁸ or —SO₂R¹⁸;
R¹⁷ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, methylaminomethylalkenyl, dimethylaminomethylalkenyl, alkynyl, haloalkynyl, cyanoalkynyl, hydroxymethylalkynyl, alkoxymethylalkynyl, aminomethylalkynyl, methylaminomethylalkynyl, dimethylaminomethylalkynyl, alkoxy, haloalkoxy, alkenyloxy, alkynyloxy, —NH—(CH₂)₂—R¹⁹, —O—(CH₂)₂—R¹⁹ or —NR²⁰R²¹; where R¹⁹ is —OR⁹ or —NR⁹R¹⁰, and R²⁰ and R²¹ are each independently H, alkyl, cycloalkyl or haloalkyl; and
R¹⁸ is alkyl, haloalkyl, cyanoalkyl, hydroxyalkyl, alkoxyalkyl, aminoalkyl, alkenyl, haloalkenyl, cyanoalkenyl, hydroxymethylalkenyl, alkoxymethylalkenyl, aminomethylalkenyl, alkynyl, haloalkynyl, cyanoalkynyl, hydroxymethylalkynyl, alkoxymethylalkynyl, aminomethylalkynyl, or —NR⁹R¹⁰.

10. The compound of claim 1 that has Formula Ia: 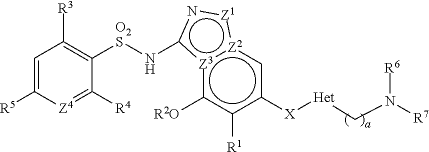
or a pharmaceutically acceptable derivative thereof.

11. The compound of claim 1, wherein:
R¹ is H;
R² is alkyl, cycloalkyl or haloalkyl;
R⁸ is H, CN, OH, aryl, heteroaryl, NR⁹R¹⁰ or C(O)R¹;
R⁹ and R¹⁰ are each independently H or C(O)R¹³; where R¹³ is alkyl or alkoxy;
R¹¹ is alkyl or alkoxy;
X is CH₂; and
R¹⁷ is alkoxy.

12. The compound of claim 1, wherein:
R¹ is H;
R² is alkyl or haloalkyl;
R⁸ is H, CN, OH, aryl, NR⁹R¹⁰ or C(O)R¹¹;
R⁹ and R¹⁰ are each independently H or C(O)R¹³; where R¹³ is alkyl;
R¹¹ is alkoxy;
X is CH₂; and
R¹⁷ is alkoxy.

13. The compound of claim 1, wherein X is CH₂.

14. The compound of claim 1, wherein a is 0 or 1.

15. The compound of claim 1, wherein R² is alkyl.

16. The compound of claim 1, wherein R¹ is H.

17. The compound of claim 1, wherein b is 1 or 2.

18. The compound of claim 1, wherein R⁸ is H, phenyl, 4-fluorophenyl, mopholinyl, COOH, COOMe, C(═NH)NHOH, tetrazolyl, oxadiazolyl, triazolyl, pyridyl, imidazolyl, oxazolidinyl, NHC(O)OMe, cyano, hydroxy, amino or acetylamino.

19. The compound of claim 1, wherein R⁸ is hydrogen.

20. The compound of claim 1, wherein R⁴ is H.

21. The compound of claim 1, wherein Z¹ is NH or O, and Z² and Z³ are C.

22. The compound of claim 1, wherein Z¹ is O, and Z² and Z³ are C.

23. The compound of claim 1, wherein Z⁴ is CR¹⁶.

24. The compound of claim 1, wherein Het is a 5- or 6-membered heteroaryl, and R⁶ is H or alkyl.

25. The compound of claim 1, wherein Het is a 5-membered heteroaryl, and R⁶ is H or methyl.

26. The compound of claim 1, wherein Het is pyrazolyl, oxazolyl, thiazolyl or pyridyl, and R⁶ is H or methyl.

27. The compound of claim 1, wherein Het is pyrazolyl, and R⁶ is H or methyl.

28. The compound of claim 1, wherein R⁷ is —C(O)R¹⁷.

29. The compound of claim 1, wherein R¹⁷ is alkyl, alkoxy, alkenyl, haloalkenyl, alkynyl, NR²⁰R²¹ or hydroxyalkynyl.

30. The compound of claim 1, wherein R¹⁷ is methyl, methoxy, NHMe, ethenyl, 1-fluoro-1-ethenyl, ethynyl or 1-propyn-1-yl.

31. A pharmaceutical composition, comprising the compound of any one of claims 1-9 and a pharmaceutically acceptable carrier.

32. A method of treating a subject having cancer, comprising administering to the subject the compound of any one of claims 1-9.

33. A method of treating a subject having a myc-driven cancer, comprising administering to the subject the compound of any one of claims 1-9.
